(19) United States
(12) Patent Application Publication
(10) Pub. No.: US 2024/0190821 A1
(43) Pub. Date: Jun. 13, 2024

(54) SULFONAMIDE SUBSTITUTED N-(1H-INDOL-7-YL)BENZENESULFONAMIDES AND USES THEREOF

(71) Applicant: Triana Biomedicines, Inc., Lexington, MA (US)

(72) Inventors: William J. Greenlee, Teaneck, NJ (US); Nicholas Calandra, Weston, MA (US); Soumya Ray, Quincy, MA (US); Arthur F. Kluge, Gainesville, FL (US)

(21) Appl. No.: 18/275,382

(22) PCT Filed: Feb. 1, 2022
(86) PCT No.: PCT/US2022/014723

§ 371 Date: Aug. 1, 2023

Related U.S. Application Data
(60) Provisional application No. 63/144,580, filed on Feb. 2, 2021.

(51) Int. Cl. C07D 209/40; C07D 401/12; C07D 403/12; C07D 405/12; C07D 471/04; C07D 471/18; C07D 487/04; C07D 487/10; C07D 498/10

(52) U.S. Cl. CPC C07D 209/40; C07D 401/12; C07D 403/12; C07D 405/12; C07D 471/04; C07D 471/18; C07D 487/04; C07D 487/10; C07D 498/10

(57) ABSTRACT

The present application discloses novel compounds of formula (I), pharmaceutical compositions containing these compounds and methods of inducing degradation of a protein, comprising contacting the protein with an effective amount of a compound of the disclosure. Methods of treating disease and disorders that results from abnormal activity of a target protein in a subject, are also disclosed.

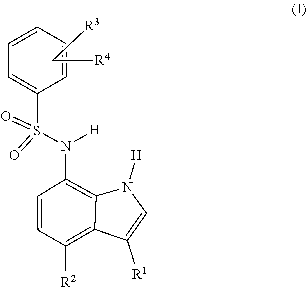

### RELATED APPLICATIONS

This application claims the benefit of priority to U.S. Provisional Application No. 63/144,580, filed Feb. 2, 2021, the entire contents of which are incorporated herein by reference.

### SUMMARY OF THE INVENTION

Various embodiments provide novel compounds, pharmaceutical compositions comprising such compounds, and methods of inducing degradation of a protein.

Some embodiments of the present disclosure are directed to a compound of Formula I

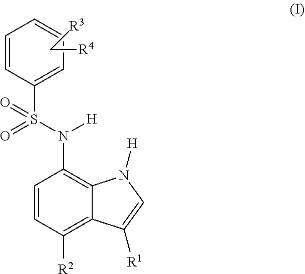

or a pharmaceutically acceptable salt thereof, wherein:
R¹ is selected from the group consisting of chloro, bromo, fluoro, and iodo;
R² is selected from the group consisting of H, chloro, fluoro, and methyl;
R³ is selected from the group consisting of H, chloro, fluoro, cyano, and methyl;
R⁴ is selected from the group consisting of
   (a)

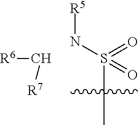

wherein:
   R⁵ is selected from the group consisting of H and C₁-C₆ alkyl;
   R⁶ is selected from the group consisting of —CO₂CH₃, —CH₂OCH₃, CH₂S(O)₂C₁-C₆ alkyl, ((optionally substituted 3 to 7-membered heterocyclyl)oxy)C₁-C₆ alkyl, (optionally substituted C₁-C₆ alkoxy)C₁-C₆ alkyl, amino C₁-C₆ alkyl, optionally substituted C₃-C₇ cycloalkyl, optionally substituted 3 to 7-membered heterocyclyl, optionally substituted C₆-C₁₀ aryl, optionally substituted 5 to 10-membered heteroaryl, (optionally substituted 3 to 7-membered heterocyclyl)C₁-C₆ alkyl, (optionally substituted C₆-C₁₀ aryl)C₁-C₆ alkyl, (optionally substituted 5 to 10-membered heteroaryl)C₁-C₆ alkyl, and

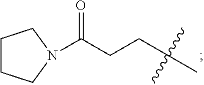 [SMILES: *CCC(=O)N1CCCC1]

R⁷ is selected from the group consisting of H, C₁-C₆ alkyl, C₃-C₇ cycloalkyl, and ((dialkylamino)carbonyl)C₁-C₆ alkyl; or
alternatively R⁶ and R⁷ together with the carbon atom to which they are attached form a group selected from an optionally substituted 4 to 7-membered heterocyclyl and optionally substituted indanyl;
(b)

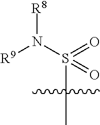

wherein
R⁸ and R⁹ together with the nitrogen atom to which they are attached form a group selected from optionally substituted 4 to 12-membered heterocyclic ring, or optionally substituted 5 to 6-membered heteroaryl ring, wherein the heterocyclic ring is a monocyclic, fused, spirocyclic or bridged heterocyclyl; with the proviso that the compound is not

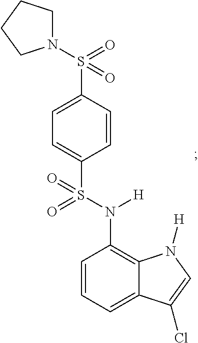

(c)

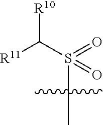

wherein
   R¹⁰ and R¹¹ together with the carbon atom to which they are attached form an optionally substituted 6-membered heterocyclyl;
(d)

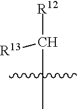

wherein
   R¹² is H;
   R¹³ is selected from the group consisting of —NHCOC₁-C₂ alkyl, —CH₂NHCOC₁-C₂ alkyl, —NHCO₂C₁-C₄ alkyl, —CH₂NHCO₂C₁-C₄ alkyl and optionally substituted triazole; or
   alternatively R¹² and R¹³ together with the carbon atom to which they are attached form a group selected from an optionally substituted C₃-C₆ cycloalkyl, optionally substituted 4 to 6-membered heterocyclyl, optionally substituted phenyl, and optionally substituted 5 to 6-membered-heteroaryl;
(e)

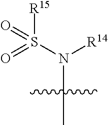

wherein:
R¹⁴ is selected from H and C₁-C₃ alkyl; and
R¹⁵ is an optionally substituted 6-membered heterocyclyl;
(f)

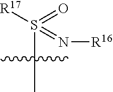

wherein
R¹⁶ is selected from the group consisting of H and C₁-C₃ alkyl; and
R¹⁷ is selected from the group consisting of C₁-C₆ alkyl, C₁-C₃ aryl and optionally substituted 6-membered heterocyclyl; and
(g)

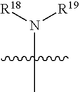

wherein
R¹⁸ and R¹⁹ together with the nitrogen to which they are attached form 5 to 6-membered heteroaryl group.

Some embodiments of the present disclosure are directed to a compound of Formula II

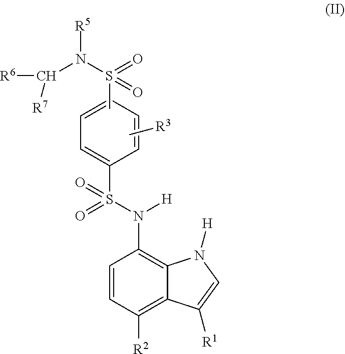

or a pharmaceutically acceptable salt thereof, wherein:
R¹ is selected from the group consisting of chloro, bromo, fluoro, and iodo;
R² is selected from the group consisting of H, chloro, fluoro and methyl;
R³ is selected from the group consisting of H, chloro, fluoro, cyano and methyl;
R⁵ is selected from the group consisting of H and C₁-C₆ alkyl;
R⁶ is selected from the group consisting of —CO₂CH₃, —CH₂OCH₃, CH₂S(O)₂C₁-C₆ alkyl, ((optionally substituted 3 to 7-membered heterocyclyl)oxy)C₁-C₆ alkyl, (optionally substituted C₁-C₆ alkoxy)C₁-C₆ alkyl, amino C₁-C₆ alkyl, optionally substituted C₃-C₇ cycloalkyl, optionally substituted 3 to 7-membered heterocyclyl, optionally substituted C₆-C₁₀ ary, optionally substituted 5 to 10-membered heteroaryl, (optionally substituted 3 to 7-membered heterocyclyl)C₁-C₆ alkyl, (optionally substituted C₆-C₁₀ aryl)C₁-C₆ alkyl, (optionally substituted 5 to 10-membered heteroaryl)C₁-C₆ alkyl, and

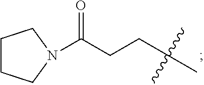 [SMILES: *CCC(=O)N1CCCC1]

R⁷ is selected from the group consisting H, C₁-C₆ alkyl, C₃-C₇ cycloalkyl, and ((dialkylamino)carbonyl)C₁-C₆ alkyl; or
alternatively R⁶ and R⁷ together with the carbon atom to which they are attached form a group selected from an optionally substituted 4 to 7-membered heterocyclyl and optionally substituted indanyl.

Some embodiments of the present disclosure are directed to a compound of Formula III

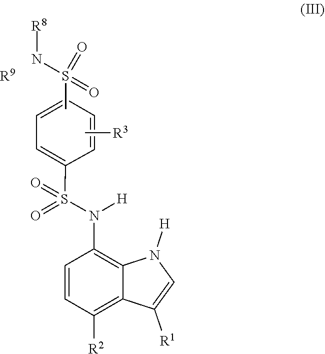

or a pharmaceutically acceptable salt thereof, wherein:
R¹ is selected from the group consisting of chloro, bromo, fluoro, and iodo;
R² is selected from the group consisting of H, chloro, fluoro and methyl;
R³ is selected from the group consisting of H, chloro, fluoro, cyano and methyl;
R⁸ and R⁹ together with the nitrogen atom to which they are attached form a group selected from optionally substituted 4 to 12-membered heterocyclic ring, or optionally substituted 5 to 6-membered heteroaryl ring, wherein the heterocyclic ring is a monocyclic, fused, spirocyclic or bridged heterocyclyl and with the proviso that the compound is not

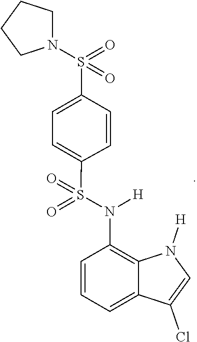 [SMILES: O=S(=O)(Nc1cccc2c(Cl)c[nH]c12)c1ccc(S(=O)(=O)N2CCCC2)cc1]

Some embodiments of the present disclosure are directed to a compound of Formula IV

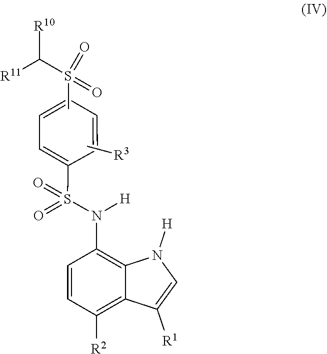

or a pharmaceutically acceptable salt thereof, wherein:
R¹ is selected from the group consisting of chloro, bromo, fluoro, and iodo;
R² is selected from the group consisting of H, chloro, fluoro and methyl;
R³ is selected from the group consisting of H, chloro, fluoro, cyano and methyl; and
R¹⁰ and R¹¹ together with the carbon atom to which they are attached form an optionally substituted 6-membered heterocyclyl.

Some embodiments of the present disclosure are directed to a compound of Formula V

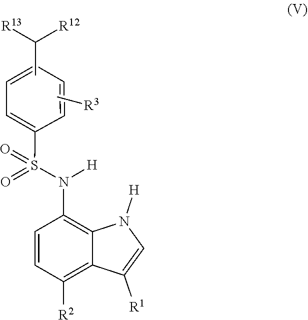

or a pharmaceutically acceptable salt thereof, wherein:
R¹ is selected from the group consisting of chloro, bromo, fluoro, and iodo;
R² is selected from the group consisting of hydrogen, chloro, fluoro and methyl;
R³ is selected from the group consisting of H, chloro, fluoro, cyano and methyl;
R¹² is H;
R¹³ is selected from the group consisting of —NHCOC₁-C₂ alkyl, —CH₂NHCOC₁-C₂ alkyl, —NHCO₂C₁-C₄ alkyl, —CH₂NHCO₂C₁-C₄ alkyl and optionally substituted triazole; or
alternatively R¹² and R¹³ together with the carbon atom to which they are attached form a group selected from optionally substituted C₃-C₆ cycloalkyl, optionally substituted 4 to 6-membered heterocyclyl, optionally substituted phenyl, and optionally substituted 5 to 6-membered-heteroaryl.

Some embodiments of the present disclosure are directed to a compound of Formula VI

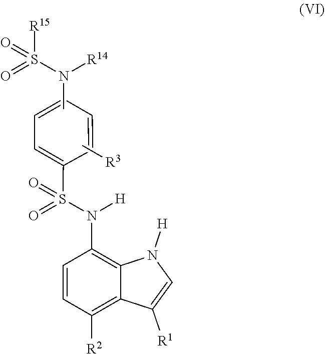

or a pharmaceutically acceptable salt thereof, wherein:
R¹ is selected from the group consisting of chloro, bromo, fluoro, and iodo;
R² is selected from the group consisting of H, chloro, fluoro and methyl;
R³ is selected from the group consisting of H, chloro, fluoro, cyano and methyl;
R¹⁴ is selected from H and C₁-C₃ alkyl; and
R¹⁵ is selected from the group consisting of optionally substituted 6-membered heterocyclyl.

Some embodiments of the present disclosure are directed to a compound of Formula VII

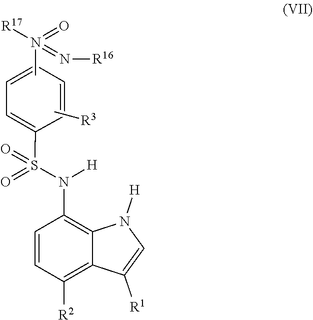

or a pharmaceutically acceptable salt thereof, wherein:
R¹ is selected from the group consisting of chloro, bromo, fluoro, and iodo;
R² is selected from the group consisting of H, chloro, fluoro and methyl;
R³ is selected from the group consisting of H, chloro, fluoro, cyano and methyl;
R¹⁶ is selected from the group consisting of H and C₁-C₃ alkyl; and
R¹⁷ is selected from the group consisting of C₁-C₃ alkyl, optionally substituted C₆-C₁₀ aryl and optionally substituted 6-membered heterocyclyl.

Some embodiments of the present disclosure are directed to a compound of Formula VIII

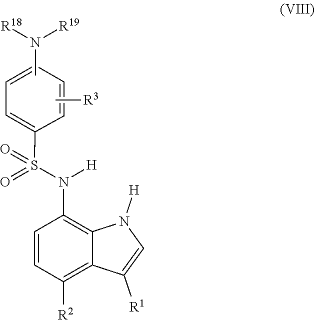

or a pharmaceutically acceptable salt thereof, wherein:
R¹ is selected from the group consisting of chloro, bromo, fluoro, and iodo;
R² is selected from the group consisting of H, chloro, fluoro and methyl;
R³ is selected from the group consisting of H, chloro, fluoro, cyano and methyl; and
R¹⁸ and R¹⁹ together with the nitrogen to which they are attached form an optionally substituted 5 to 6-membered heteroaryl group.

Some embodiments of the present disclosure are directed to a compound selected from the group consisting of:

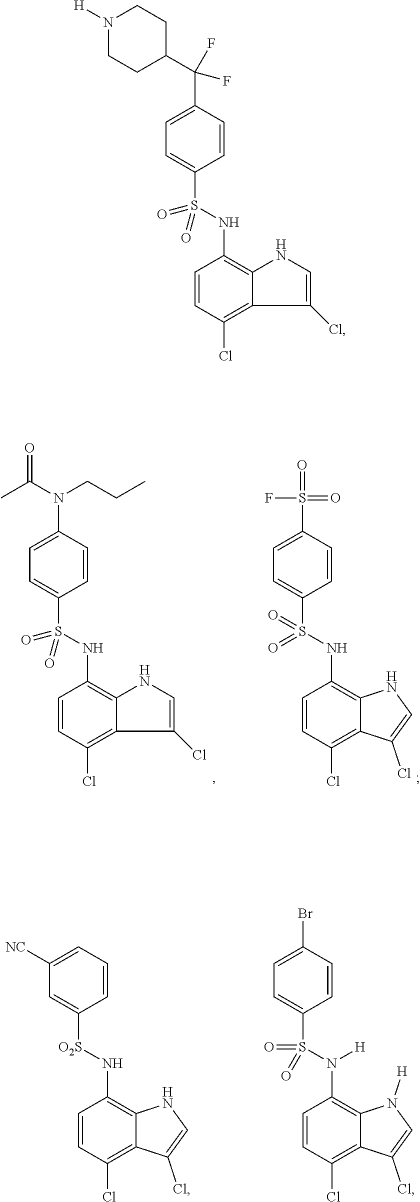 [SMILES: O=S(=O)(Nc1ccc(Cl)c2c(Cl)c[nH]c12)c1ccc(C(F)(F)C2CCNCC2)cc1 ; CCCN(C(C)=O)c1ccc(S(=O)(=O)Nc2ccc(Cl)c3c(Cl)c[nH]c23)cc1 ; O=S(=O)(F)c1ccc(S(=O)(=O)Nc2ccc(Cl)c3c(Cl)c[nH]c23)cc1 ; N#Cc1cccc(S(=O)(=O)Nc2ccc(Cl)c3c(Cl)c[nH]c23)c1 ; O=S(=O)(Nc1ccc(Cl)c2c(Cl)c[nH]c12)c1ccc(Br)cc1]

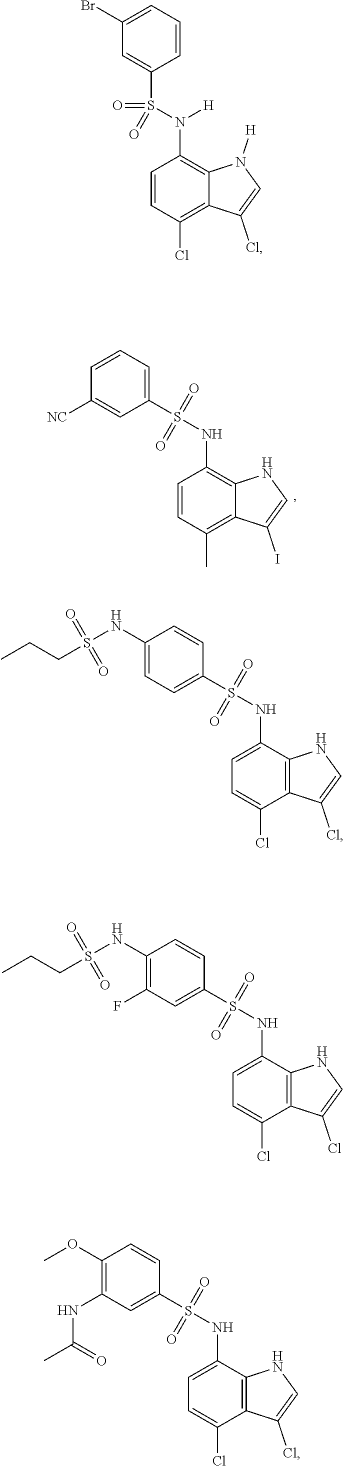 [SMILES: O=S(=O)(Nc1ccc(Cl)c2c(Cl)c[nH]c12)c1cccc(Br)c1 ; Cc1ccc(NS(=O)(=O)c2cccc(C#N)c2)c2[nH]cc(I)c12 ; CCCS(=O)(=O)Nc1ccc(S(=O)(=O)Nc2ccc(Cl)c3c(Cl)c[nH]c23)cc1 ; CCCS(=O)(=O)Nc1ccc(S(=O)(=O)Nc2ccc(Cl)c3c(Cl)c[nH]c23)cc1F ; COc1ccc(S(=O)(=O)Nc2ccc(Cl)c3c(Cl)c[nH]c23)cc1NC(C)=O]

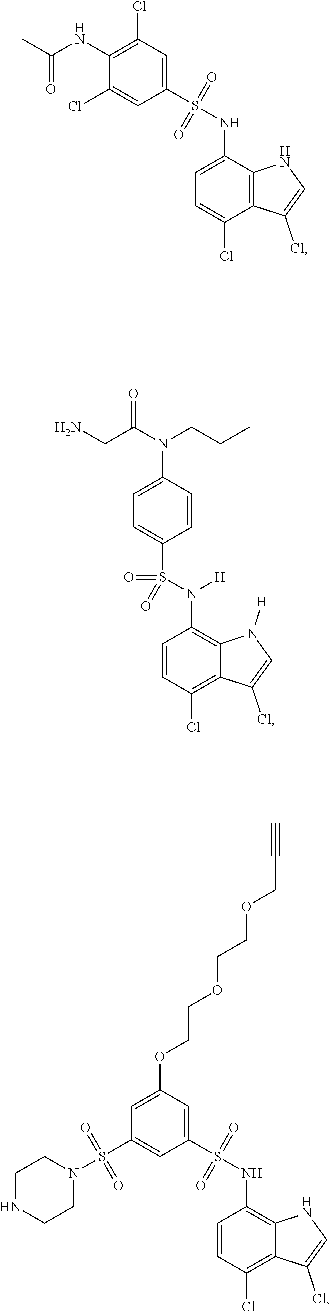 [SMILES: CC(=O)Nc1c(Cl)cc(S(=O)(=O)Nc2ccc(Cl)c3c(Cl)c[nH]c23)cc1Cl ; CCCN(C(=O)CN)c1ccc(S(=O)(=O)Nc2ccc(Cl)c3c(Cl)c[nH]c23)cc1 ; C#CCOCCOCCOc1cc(S(=O)(=O)Nc2ccc(Cl)c3c(Cl)c[nH]c23)cc(S(=O)(=O)N2CCNCC2)c1]

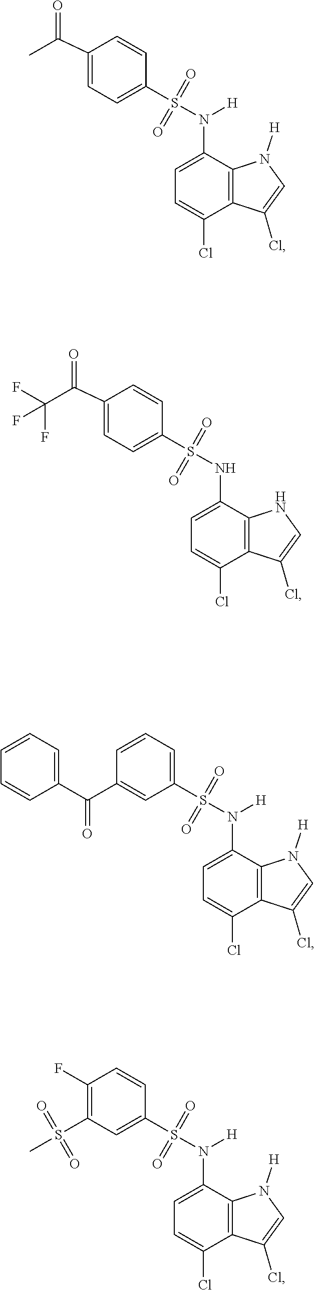 [SMILES: CC(=O)c1ccc(S(=O)(=O)Nc2ccc(Cl)c3c(Cl)c[nH]c23)cc1 ; O=C(c1ccc(S(=O)(=O)Nc2ccc(Cl)c3c(Cl)c[nH]c23)cc1)C(F)(F)F ; O=C(c1ccccc1)c1cccc(S(=O)(=O)Nc2ccc(Cl)c3c(Cl)c[nH]c23)c1 ; CS(=O)(=O)c1cc(S(=O)(=O)Nc2ccc(Cl)c3c(Cl)c[nH]c23)ccc1F]

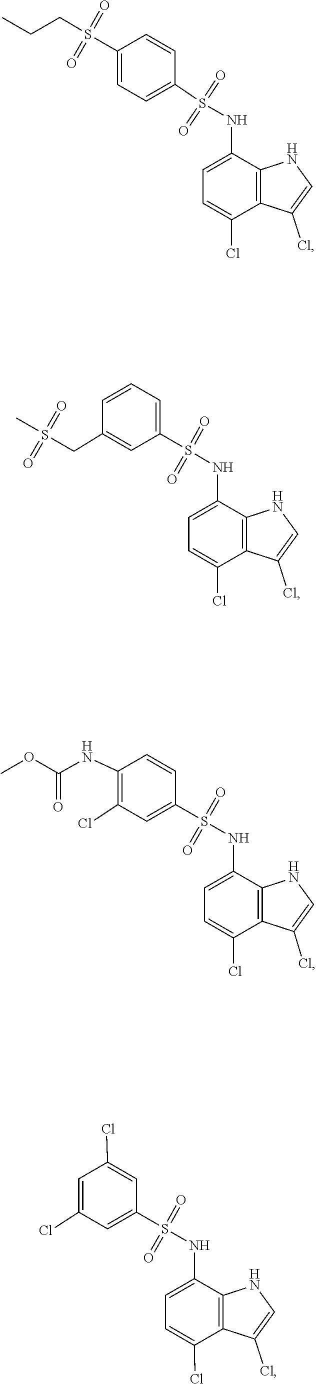 [SMILES: CCCS(=O)(=O)c1ccc(S(=O)(=O)Nc2ccc(Cl)c3c(Cl)c[nH]c23)cc1 ; CS(=O)(=O)Cc1cccc(S(=O)(=O)Nc2ccc(Cl)c3c(Cl)c[nH]c23)c1 ; COC(=O)Nc1ccc(S(=O)(=O)Nc2ccc(Cl)c3c(Cl)c[nH]c23)cc1Cl ; O=S(=O)(Nc1ccc(Cl)c2c(Cl)c[nH]c12)c1cc(Cl)cc(Cl)c1]

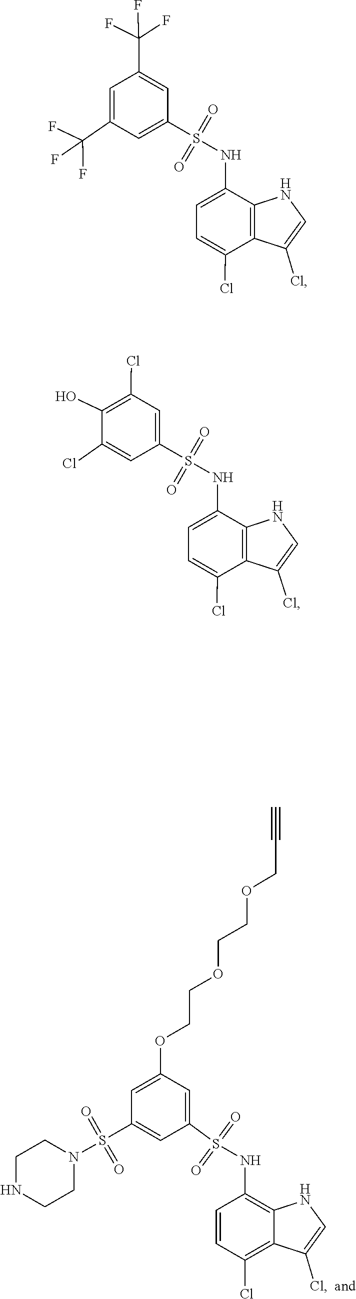

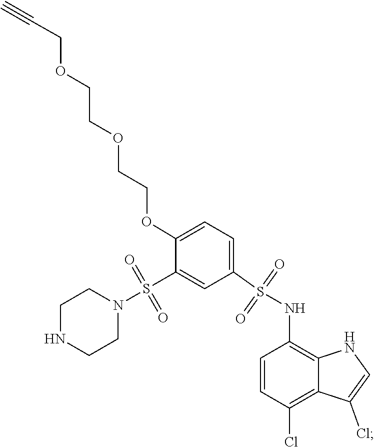 [SMILES: C#CCOCCOCCOc1ccc(S(=O)(=O)Nc2ccc(Cl)c3c(Cl)c[nH]c23)cc1S(=O)(=O)N1CCNCC1]

or a pharmaceutically acceptable salt thereof.

Some embodiments of the present disclosure are directed to compound selected from the group consisting of:

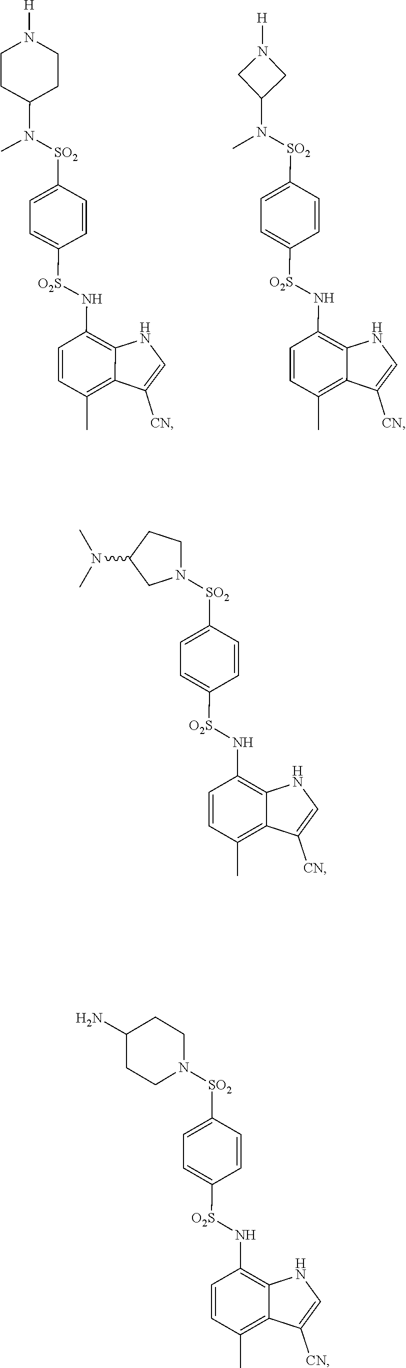 [SMILES: Cc1ccc(NS(=O)(=O)c2ccc(S(=O)(=O)N(C)C3CCNCC3)cc2)c2[nH]cc(C#N)c12 ; Cc1ccc(NS(=O)(=O)c2ccc(S(=O)(=O)N(C)C3CNC3)cc2)c2[nH]cc(C#N)c12 ; Cc1ccc(NS(=O)(=O)c2ccc(S(=O)(=O)N3CCC(N(C)C)C3)cc2)c2[nH]cc(C#N)c12 ; Cc1ccc(NS(=O)(=O)c2ccc(S(=O)(=O)N3CCC(N)CC3)cc2)c2[nH]cc(C#N)c12]

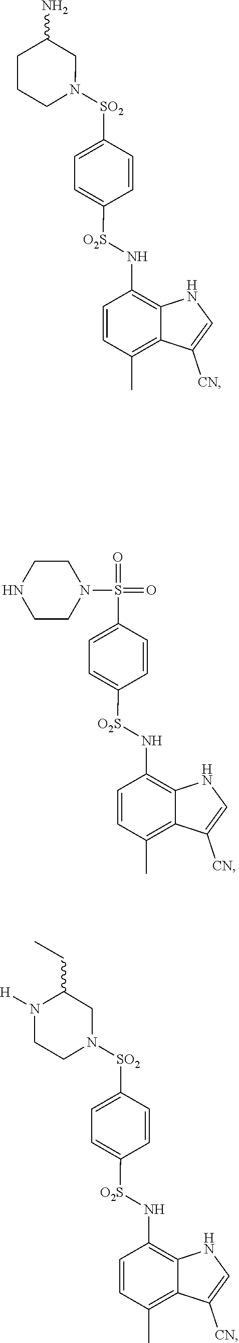 [SMILES: Cc1ccc(NS(=O)(=O)c2ccc(S(=O)(=O)N3CCCC(N)C3)cc2)c2[nH]cc(C#N)c12 ; Cc1ccc(NS(=O)(=O)c2ccc(S(=O)(=O)N3CCNCC3)cc2)c2[nH]cc(C#N)c12 ; CCC1CN(S(=O)(=O)c2ccc(S(=O)(=O)Nc3ccc(C)c4c(C#N)c[nH]c34)cc2)CCN1]

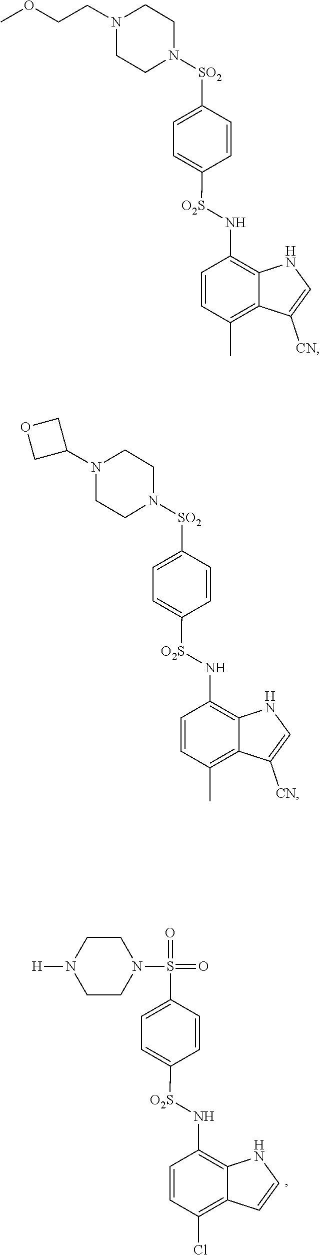 [SMILES: COCCN1CCN(S(=O)(=O)c2ccc(S(=O)(=O)Nc3ccc(C)c4c(C#N)c[nH]c34)cc2)CC1 ; Cc1ccc(NS(=O)(=O)c2ccc(S(=O)(=O)N3CCN(C4COC4)CC3)cc2)c2[nH]cc(C#N)c12 ; O=S(=O)(Nc1ccc(Cl)c2cc[nH]c12)c1ccc(S(=O)(=O)N2CCNCC2)cc1]

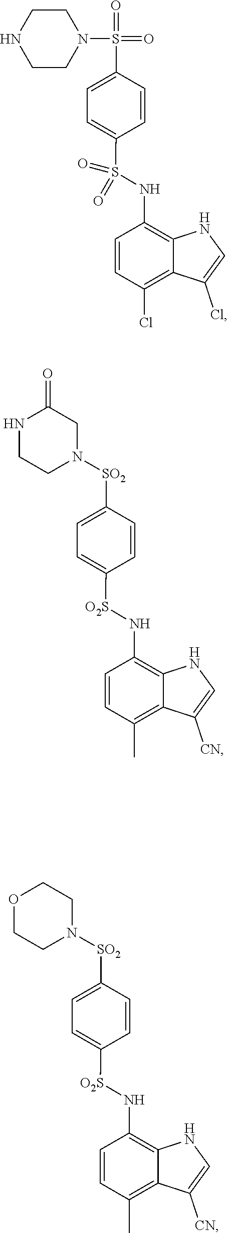 [SMILES: O=S(=O)(Nc1ccc(Cl)c2c(Cl)c[nH]c12)c1ccc(S(=O)(=O)N2CCNCC2)cc1 ; Cc1ccc(NS(=O)(=O)c2ccc(S(=O)(=O)N3CCNC(=O)C3)cc2)c2[nH]cc(C#N)c12 ; Cc1ccc(NS(=O)(=O)c2ccc(S(=O)(=O)N3CCOCC3)cc2)c2[nH]cc(C#N)c12]

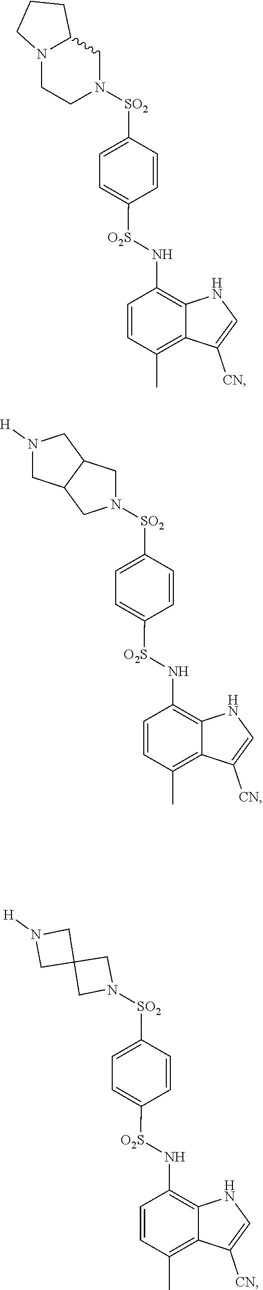 [SMILES: Cc1ccc(NS(=O)(=O)c2ccc(S(=O)(=O)N3CCN4CCCC4C3)cc2)c2[nH]cc(C#N)c12 ; Cc1ccc(NS(=O)(=O)c2ccc(S(=O)(=O)N3CC4CNCC4C3)cc2)c2[nH]cc(C#N)c12 ; Cc1ccc(NS(=O)(=O)c2ccc(S(=O)(=O)N3CC4(CNC4)C3)cc2)c2[nH]cc(C#N)c12]

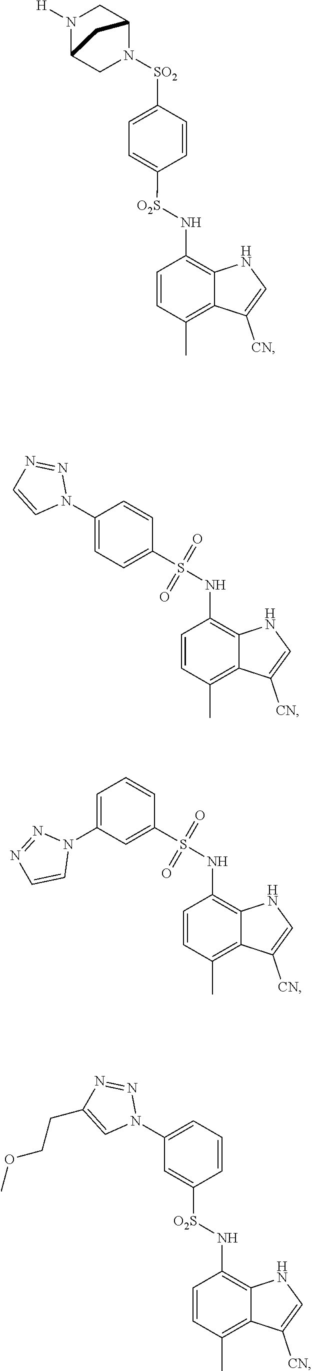

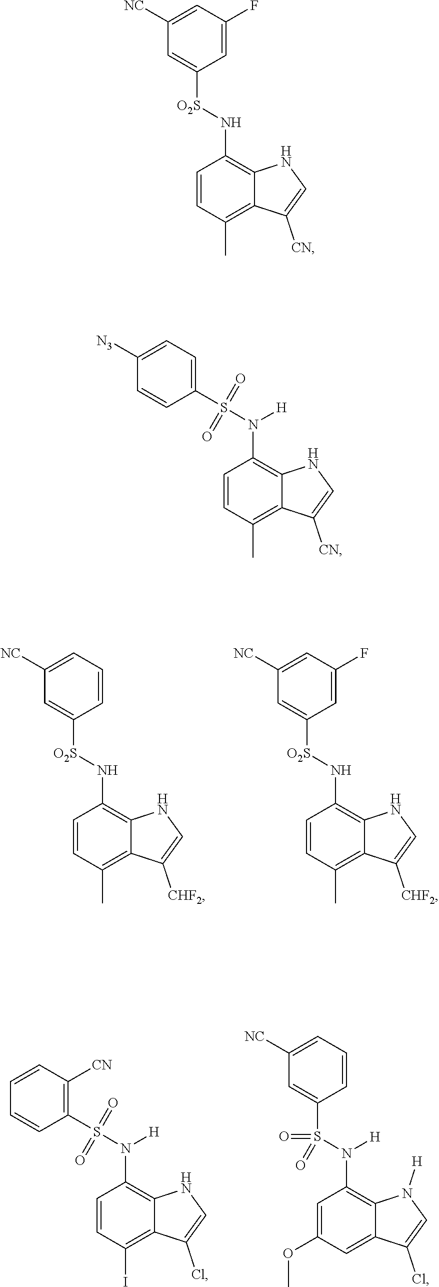 [SMILES: Cc1ccc(NS(=O)(=O)c2cc(F)cc(C#N)c2)c2[nH]cc(C#N)c12 ; Cc1ccc(NS(=O)(=O)c2ccc(N=[N+]=[N-])cc2)c2[nH]cc(C#N)c12 ; Cc1ccc(NS(=O)(=O)c2cccc(C#N)c2)c2[nH]cc(C(F)F)c12 ; Cc1ccc(NS(=O)(=O)c2cc(F)cc(C#N)c2)c2[nH]cc(C(F)F)c12 ; N#Cc1ccccc1S(=O)(=O)Nc1ccc(I)c2c(Cl)c[nH]c12 ; COc1cc(NS(=O)(=O)c2cccc(C#N)c2)c2[nH]cc(Cl)c2c1]

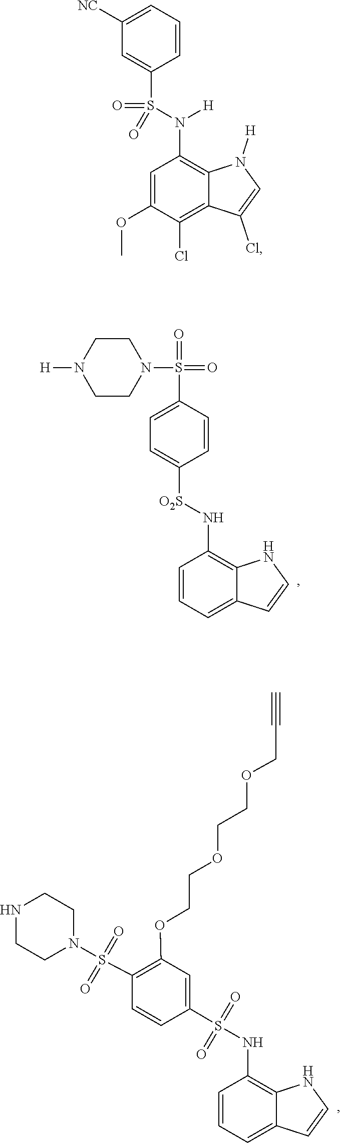 [SMILES: COc1cc(NS(=O)(=O)c2cccc(C#N)c2)c2[nH]cc(Cl)c2c1Cl ; O=S(=O)(Nc1cccc2cc[nH]c12)c1ccc(S(=O)(=O)N2CCNCC2)cc1 ; C#CCOCCOCCOc1cc(S(=O)(=O)Nc2cccc3cc[nH]c23)ccc1S(=O)(=O)N1CCNCC1]

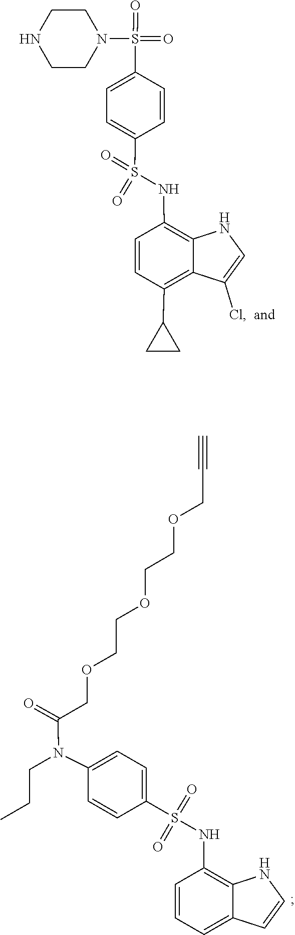

or a pharmaceutically acceptable salt thereof.

Some embodiments of the present disclosure are directed to a method of inducing degradation of a protein, comprising contacting the protein with an effective amount of a compound or composition according to embodiments described herein.

Some embodiments of the present disclosure are directed to a method of inducing degradation of a protein, in a patient in need thereof, comprising administering to the patient, a therapeutically effective amount of a compound according to embodiments herein.

Some embodiments of the present disclosure are directed to a method of treating a disease or disorder that results from abnormal activity of a target protein in a subject, comprising administering to the subject, a therapeutically effective amount of a compound according to embodiments herein, wherein the compound induces degradation of the protein thereby treating the disease or disorder.

Some embodiments of the present disclosure are directed to a method of treating a disease or disorder that results from abnormal expression of a gene that encodes a target protein in a subject, comprising administering to the subject, a therapeutically effective amount of a compound according to embodiments herein, wherein the compound induces degradation of the protein thereby treating the disease or disorder.

### DETAILED DESCRIPTION

Molecular glues degraders are small molecules that bring about targeted protein degradation by promoting the association of a target protein with a ubiquitin E3 ligase. This results in ubiquitination of the target protein, followed by its degradation by the proteasome. During protein degradation, the molecular glue dissociates, freeing it up to form a new target protein—E3 ligase complex. Examples of molecular glues include the IMiDs (immune modulatory drugs; e.g., thalidomide), which promote a novel interaction between a substrate (e.g., IKZF1/3) and cereblon, a substrate receptor (also known as DCAF) for Cullin-RING ubiquitin ligase 4 (CRL4). More recently, the small molecule indisulam was reported to be a molecular glue that enhances the binding of DCAF15, another CRL4 substrate receptor, to a novel substrate, the pre-mRNA splicing factor RBM39, promoting its degradation.

Molecular glues have several advantages over other therapeutic modalities for treating a variety of diseases and disorders. By using the function of specific effector proteins, in this case a ligase in a cell to eliminate a protein of interest, or target can provide highly specific drug therapy options with fewer side effects, such as off target effects and/or toxicity. Furthermore, more than three quarters of human proteins have remained beyond the reach of therapeutic development, despite enormous efforts to advance traditional pharmacology approaches. Compounds that bring about targeted protein degradation offer one approach that can overcome this limitation. Thus, there is a need for new compounds that function as molecular glues and induce protein degradation for the treatment of diseases and disorders.

### Definitions

Unless defined otherwise, all technical and scientific terms used herein have the same meanings as commonly understood by one of ordinary skill in the art.

The articles “a” and “an” as used herein mean “one or more” or “at least one,” unless otherwise indicated. That is, reference to any element of the present invention by the indefinite article “a” or “an” does not exclude the possibility that more than one of the element is present.

As used herein, the term “about” means plus or minus 10% of the numerical value of the number with which it is being used. Therefore, about 50% means in the range of 45%-55%.

“Administering,” or “administration” and the like, when used in conjunction with the compounds of the disclosure refers to providing the compounds or pharmaceutical compositions according to any of the embodiments described herein, to a subject in need of treatment. Preferably the subject is a mammal, more preferably a human. In one aspect, the present invention comprises administering the compound or pharmaceutical composition of the invention alone or in conjunction with another therapeutic agent. When a compound or pharmaceutical composition of the invention is administered in conjunction with another therapeutic agent, the compound or pharmaceutical composition of the invention and the other therapeutic agent can be administered at the same time or different times, and by the same routes of administration or by different routes of administration.

As used herein, the term “alkyl” is intended to include both branched and straight-chain saturated aliphatic hydrocarbon groups having the specified number of carbon atoms. For example, “C₁-C₆ alkyl” or “C₁₋₆ alkyl” denotes alkyl having 1 to 6 carbon atoms. Where specified, alkyl group can be substituted with at least one hydrogen being replaced by another chemical group. In some embodiments the one of more hydrogen atoms is replaced by a chemical group selected from any group as disclosed herein. Examples of alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl). Examples of substituted alkyl includes, but is not limited to, —CH₂N(CH₃)₂, —CH₂CH₂N(CH₃)₂, and —CH₂CH₂CH₂N(CH₃)₂.

The term “animal” as used herein includes, but is not limited to, humans and non-human vertebrates such as wild, domestic and farm animals.

The term “alkoxy” or “alkyloxy” refers to an —O-alkyl group. “C₁-C₆ alkoxy” or “C₁₋₆ alkoxy” (or alkyloxy), is intended to include C₁, C₂, C₃, C₄, C₅, and C₆, alkoxy groups. The alkoxy group can be unsubstituted or substituted with at least one hydrogen being replaced by another chemical group. In some embodiments the one of more hydrogen atoms is replaced by a chemical group selected from any group as disclosed herein. Examples of alkoxy groups include, but are not limited to, methoxy, ethoxy, propoxy (e.g., n-propoxy and isopropoxy), and t-butoxy.

The term “alkoxyalkyl” denotes an alkoxy group bound to an alkyl radical. For example —CH₂OCH₃ is the alkoxyalkyl group methoxymethyl. In one aspect, an alkoxy group can substituted with at least one hydrogen being replaced by another chemical group. In some embodiments the one of more hydrogen atoms is replaced by a chemical group selected from any group as disclosed herein.

“Aryl” groups refer to monocyclic or polycyclic aromatic hydrocarbons, including, for example, phenyl, and naphthyl. “C₆-C₁₀ aryl” or “C₆-10 aryl” refers to phenyl and naphthyl. In one aspect, “aryl”, “C₆-C₁₀ aryl,” “C₆₋₁₀ aryl,” or “aromatic residue” may be substituted with 1 to 5 groups selected from —OH, —OCH₃, —Cl, —F, —Br, —I, —CN, —NO₂, —NH₂, —NH(CH₃), —N(CH₃)₂, —CF₃, —OCF₃, —C(O)CH₃, —SCH₃, —S(O)CH₃, —S(O)₂CH₃, —CH₃, —CH₂CH₃, —CO₂H, —CO₂CH₃ or by any chemical group as defined herein.

The term “arylalkyl” refers to an aryl group bound to an alkyl radical. For example —CH₂CH₂(C₆H₄) is the arylalkyl group phenylethyl. Benzyl is another example of an arylalkyl group. In one aspect, the benzyl group can be substituted with at least one hydrogen being replaced by another chemical group. In some embodiments the one of more hydrogen atoms is replaced by a chemical group selected from any group as disclosed herein.

The term “benzyl”, as used herein, refers to a methyl group on which one of the hydrogen atoms is replaced by a phenyl group, wherein said phenyl group may optionally be substituted by one to five, preferably one to three, substituents independently selected from methyl, trifluoromethyl (—CF₃), hydroxyl (—OH), methoxy (—OCH₃), halogen, cyano (—CN), nitro (—NO₂), —CO₂Me, —CO₂Et, and —CO₂H, or any group as disclosed herein. Representative examples of benzyl group include, but are not limited to, PhCH₂—, 4-MeO—C₆H₄CH₂—, 2,4,6-tri-methyl-C₆H₂CH₂—, and 3,4-di-C₁-C₆H₃CH₂—.

“Benzyloxy” refers to the group —OCH₂(C₆H₄). In one aspect, a benzyloxy group can be substituted with at least one hydrogen being replaced by another chemical group, for example, any group disclosed herein.

The term “benzyloxy(alkyl)”, as used herein, refers to a benzyloxy group bound to an alkyl radical. For example —CH₂OCH₂(C₆H₄) is a benzyloxymethyl group. In one aspect, a benzyloxymethyl group can be substituted with at least one hydrogen being replaced by another chemical group, for example, any group disclosed herein.

The term “cycloalkyl” or “cycloalkyl ring” is defined as a saturated or partially unsaturated carbocyclic ring in a single or fused carbocyclic ring system having from three to twelve ring members. In a preferred embodiment, a cycloalkyl is a ring system having three to seven ring members. In one apsect, one or more hydrogen atoms may also be replaced by a substituent group selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl, formyl or any other group as disclosed herein. Examples of a cycloalkyl group include, without limitation, cyclopropyl, cyclobutyl, cyclohexyl, and cycloheptyl.

The term “(cycloalkyl)alkoxyl” refers to an oxygen radical bound to a cycloalkyl group. For example, —O—(C₆H₁₁) is a cyclohexylalkoxy group. In one aspect, a (cycloalkyl)alkoxy group can be unsubstituted or substituted with at least one hydrogen being replaced by another chemical group, for example, any group disclosed herein.

The term “(cycloalkyl)alkoxyalkyl refers to a cycloalkoxy group bound to an alkyl radical. In one aspect, a (cycloalkyl)alkoxyalkyl group can be substituted with at least one hydrogen being replaced by another chemical group, for example, any group disclosed herein.

A “dialkylaminocarbonyl” or “caboxamido” denotes a carbonyl radical adjacent to an dialkylamino group. In one aspect, a dialkylaminocarbonyl group can be substituted with at least one hydrogen being replaced by another chemical group, for example, any group disclosed herein.

The terms “formula” and “structure” are used interchangeably herein.

The term “halo” or “halogen” refers to fluoro, chloro, bromo, and iodo.

As used herein, the term “heteroaryl” is intended to mean stable monocyclic and polycyclic aromatic hydrocarbons that include at least one heteroatom ring member, such as sulfur, oxygen, or nitrogen.

Heteroaryl groups include, without limitation, pyridinyl, pyrimidinyl, pyrazinyl, pyridazinyl, triazinyl, furanyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-oxadiazolyl, 1,2,4-thiadiazolyl, isothiazolyl, purinyl, carbazolyl, benzimidazolyl, and indolinyl. Unless otherwise specified, heteroaryl groups may be unsubstituted or substituted with 1 to 5 groups selected from —OH, —OCH₃, —Cl, —F, —Br, —I, —CN, —NO₂, —NH₂, —NH(CH₃), —N(CH₃)₂, —CF₃, —OCF₃, —C(O)CH₃, —SCH₃, —S(O)CH₃, —S(O)₂CH₃, —CH₃, —CH₂CH₃, —CO₂H, —CO₂CH₃, as well as any other group disclosed herein. The nitrogen atom is substituted or unsubstituted (i.e., N or NR wherein R is H or another substituent, if defined). The nitrogen and sulfur heteroatoms may optionally be oxidized (i.e., N→0 and S(O)ₚ, wherein p is 0, 1 or 2).

As used herein, the term “heteroarylalkyl” denote a heteroaryl group adjacent to an alkyl radical.

The term “heterocyclyl,” “heterocyclic” or “heterocyclyl ring” is defined as a saturated or partially unsaturated ring containing one to four hetero atoms or hetero groups selected from O, N, NH, —N(R^{Z})—, —S(O)— or —S(O)₂—, wherein R^{Z} is selected from alkyl, alkenyl, alkynyl, aryl, heteroaryl, cycloalkyl, in a single, fused, spiro or bridged heterocyclic ring system having from three to twelve ring members. unsubstituted or substituted with any group as defined herein. In a preferred embodiment, a heterocyclyl is a ring system having three to seven ring members. Examples of a heterocyclyl group include, without limitation, azapan-2-on-yl, azetidinyl, diazacyclohexyl, morpholinyl, oxanyl, piperidinyl, piperidin-2-on-yl, piperazinyl, piperazin-2-one, benzodioxolanyl, benzodioxanyl, and pyrrolidinyl.

The term “heterocyclyloxy refers to a heterocyclyl bound to an oxygen radical.

The phrase “molecular glue” as used herein is used to describe a compound that induces an interaction between a substrate receptor of an E3 ligase and a target protein, leading to degradation of the protein.

The phrase “pharmaceutically acceptable” refers to those compounds, materials, pharmaceutical compositions, and/or dosage forms that are, within the scope of sound medical judgment, generally regarded as safe and nontoxic. In particular, pharmaceutically acceptable carriers, diluents or other excipients used in the pharmaceutical compositions of this disclosure are physiologically tolerable, compatible with other ingredients, and do not typically produce an allergic or similar untoward reaction (for example, gastric upset, dizziness and the like) when administered to a patient. Preferably, as used herein, the term “pharmaceutically acceptable” means approved by a regulatory agency of the Federal government or a state government or listed in the U.S. Pharmacopoeia or other generally recognized pharmacopoeia for use in animals, and more particularly in humans.

As used herein, the term “pharmaceutically acceptable carrier” is a substrate used to deliver the compounds of the invention. Carriers as used herein include excipients and diluents and may depend upon the mode of administration of the compounds as described herein. Carriers for example may be edible carriers, liquid carries, carriers that will protect the compounds against rapid elimination from the body, as well as liposomal suspensions. Carriers are well known in the art and include, for example, phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

As used herein, “pharmaceutically acceptable salts” refer to non-toxic acid or base salts of the disclosed compounds. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic. The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, PA, 1990, the disclosure of which is hereby incorporated by reference.

The terms “subject,” “individual” or “patient” are used interchangeably and as used herein are intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients. The methods are particularly suitable for treating human patients having a condition, disease or disorder described herein.

As used herein, the term “therapeutic” means an agent utilized to treat, combat, ameliorate, prevent or improve an unwanted condition or disease of a patient.

A “therapeutically effective amount” of a compound, pharmaceutically acceptable salt thereof or pharmaceutical composition according to any embodiment described herein, is an amount sufficient to produce a selected effect on at least one symptom or parameter of a specific disease or disorder. The therapeutic effect may be objective (i.e., measurable by some test or marker) or subjective (i.e., subject gives an indication of or feels an effect or physician observes a change). The effect contemplated herein, includes both medical therapeutic and/or prophylactic treatment, as appropriate. The specific dose of a compound administered according to this disclosure to obtain therapeutic and/or prophylactic effects is determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the patient; the time of administration, route of administration, and rate of excretion of the specific compound employed and the duration of the treatment. The therapeutically effective amount administered will be determined by the physician in the light of the foregoing relevant circumstances and the exercise of sound medical judgment. A therapeutically effective amount of a compound, according to any embodiment described herein, is typically an amount such that when it is administered in a physiologically tolerable excipient composition, it is sufficient to achieve an effective systemic concentration or local concentration in the tissue.

The terms “treat,” “treated,” or “treating” as used herein, refers to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment. In one aspect, the treatment methods described herein are therapeutic treatments.

### Compounds

Some embodiments of the present disclosure describe a compound Formula I

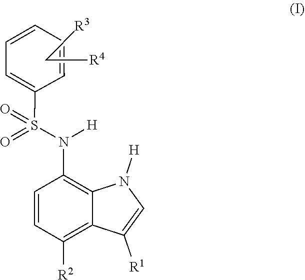

or a pharmaceutically acceptable salt thereof, wherein:
R¹ is selected from the group consisting of chloro, bromo, fluoro, and iodo;
R² is selected from the group consisting of H, chloro, fluoro, and methyl;
R³ is selected from the group consisting of H, chloro, fluoro, cyano, and methyl;
R⁴ is selected from the group consisting of
   (a)

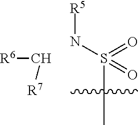

wherein:
   R⁵ is selected from the group consisting of H and C₁-C₆ alkyl;
   R⁶ is selected from the group consisting of —CO₂CH₃, —CH₂OCH₃, CH₂S(O)₂C₁-C₆ alkyl, ((optionally substituted 3 to 7-membered heterocyclyl)oxy)C₁-C₆ alkyl, (optionally substituted C₁-C₆ alkoxy)C₁-C₆ alkyl, amino C₁-C₆ alkyl, optionally substituted C₃-C₇ cycloalkyl, optionally substituted 3 to 7-membered heterocyclyl, optionally substituted C₆-C₁₀ aryl, optionally substituted 5 to 10-membered heteroaryl, (optionally substituted 3 to 7-membered heterocyclyl)C₁-C₆ alkyl, (optionally substituted C₆-C₁₀ aryl)C₁-C₆ alkyl, (optionally substituted 5 to 10-membered heteroaryl)C₁-C₆ alkyl, and

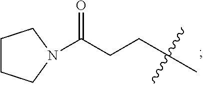 [SMILES: *CCC(=O)N1CCCC1]

R⁷ is selected from the group consisting of H, C₁-C₆ alkyl, C₃-C₇ cycloalkyl, and ((dialkylamino)carbonyl)C₁-C₆ alkyl; or
alternatively R⁶ and R⁷ together with the carbon atom to which they are attached form a group selected from an optionally substituted 4 to 7-membered heterocyclyl and optionally substituted indanyl;
(b)

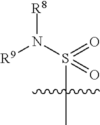

wherein
R⁸ and R⁹ together with the nitrogen atom to which they are attached form a group selected from optionally substituted 4 to 12-membered heterocyclic ring, or optionally substituted 5 to 6-membered heteroaryl ring, wherein the heterocyclic ring is a monocyclic, fused, spirocyclic or bridged heterocyclyl; with the proviso that the compound is not

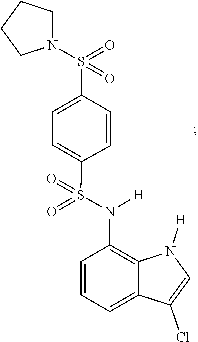

(c)

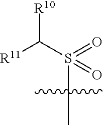

wherein
   R¹⁰ and R¹¹ together with the carbon atom to which they are attached form an optionally substituted 6-membered heterocyclyl;
(d)

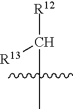

wherein
   R¹² is H;
   R¹³ is selected from the group consisting of —NHCOC₁-C₂ alkyl, —CH₂NHCOC₁-C₂ alkyl, —NHCO₂C₁-C₄ alkyl, —CH₂NHCO₂C₁-C₄ alkyl and optionally substituted triazole; or
   alternatively R¹² and R¹³ together with the carbon atom to which they are attached form a group selected from an optionally substituted C₃-C₆ cycloalkyl, optionally substituted 4 to 6-membered heterocyclyl, optionally substituted phenyl, and optionally substituted 5 to 6-membered-heteroaryl;
(e)

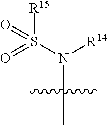

wherein:
R¹⁴ is selected from H and C₁-C₃ alkyl; and
R¹⁵ is an optionally substituted 6-membered heterocyclyl;
(f)

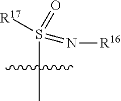

wherein
R¹⁶ is selected from the group consisting of H and C₁-C₃ alkyl; and
R¹⁷ is selected from the group consisting of C₁-C₆ alkyl, C₁-C₃ aryl and optionally substituted 6-membered heterocyclyl; and
(g)

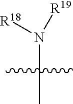

wherein
R¹⁸ and R¹⁹ together with the nitrogen to which they are attached form 5 to 6-membered heteroaryl group.

### Compounds of Formula II

In some embodiments the compound of Formula I is a compound of Formula II

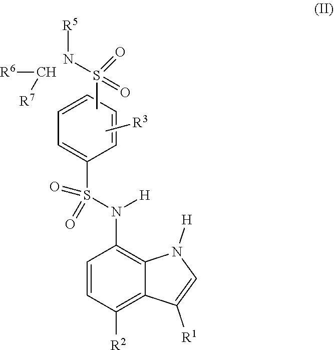

or a pharmaceutically acceptable salt thereof.

Substituent R¹ of Formula II is selected from the group consisting of chloro, bromo, fluoro, and iodo. In some embodiments, R¹ is selected from the group consisting of chloro, bromo, and iodo. In some embodiments, R¹ is chloro.

Substituent R² of Formula II is selected from the group consisting of H, chloro, fluoro and methyl. In some embodiments R² of Formula II is selected from the group consisting of hydrogen and chloro.

Substituent R³ of Formula II is selected from the group consisting of H, chloro, fluoro, cyano and methyl. In some embodiments R³ is selected from the group consisting H and F. In some embodiments, R³ is H. In some embodiments, R³ is F.

Substituent R⁵ of Formula II is selected from the group consisting of H and C₁-C₆ alkyl. In some embodiments, R⁸ is selected from the group consisting of H and C₁-C₃ alkyl.

Substituent R⁶ is selected from the group consisting of —CO₂CH₃, —CH₂OCH₃, CH₂S(O)₂C₁-C₆ alkyl, ((optionally substituted 3 to 7-membered heterocyclyl)oxy)C₁-C₆ alkyl, (optionally substituted C₁-C₆ alkoxy)C₁-C₆ alkyl, amino C₁-C₆ alkyl, optionally substituted C₃-C₇ cycloalkyl, optionally substituted 3 to 7-membered heterocyclyl, optionally substituted 6 to 10-membered aryl, optionally substituted 5 to 10-membered heteroaryl, (optionally substituted 3 to 7-membered heterocyclyl)C₁-C₆ alkyl, (optionally substituted C₆-C₁₀ aryl)C₁-C₆ alkyl, (optionally substituted 5 to 10-membered heteroaryl)C₁-C₆ alkyl, and

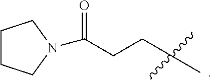 [SMILES: *CCC(=O)N1CCCC1]

In some embodiments, R⁶ is selected from the group consisting of —CO₂CH₃, —CH₂OCH₃, CH₂S(O)₂C₁-C₂ alkyl, ((optionally substituted 6-membered heterocyclyloxy)methyl, (optionally substituted C₁-C₂ alkoxy)C₁-C₃ alkyl, aminoC₁-C₆ alkyl, optionally substituted C₃-C₄ cycloalkyl, optionally substituted 6-membered heterocyclyl, optionally substituted phenyl, optionally substituted 5 to 9 membered heteroaryl, (optionally substituted 4-membered heterocyclyl)methyl, (optionally substituted phenyl)C₁-C₂ alkyl, (optionally substituted 5 to 6-membered heteroaryl)methyl, and

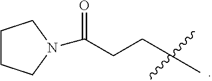 [SMILES: *CCC(=O)N1CCCC1]

In some embodiments R⁶ is selected from the group consisting of —CO₂CH₃, —CH₂OCH₃, CH₂S(O)₂ethyl, (benzyloxy)C₁-C₃ alkyl, ((cycloalkyl)C₁-C₂ alkoxy))C₁-C₃ alkyl, ((C₁-C₆ alkoxy)-C₁-C₂ alkoxy))C₁-C₃ alkyl, aminoC₁-C₆ alkyl, optionally substituted cyclopropyl, bicyclo[4.2.0]octa-1(6),2,4-trien-yl, optionally substituted piperidinyl, optionally substituted phenyl, benzo-1,4-dioxanyl, optionally substituted pyrazolyl, optionally substituted pyridinyl, optionally substituted pyrimidinyl, optionally substituted 1,2,4-oxadiazolyl, optionally substituted benzamidazolyl, optionally substituted azetidinylmethyl, (optionally substituted phenyl)methyl, (optionally substituted phenyl)ethyl, optionally substituted furanyl)methyl, (optionally substituted pyridinyl)methyl

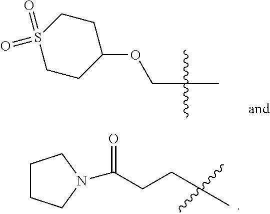 [SMILES: *COC1CCS(=O)(=O)CC1 ; *CCC(=O)N1CCCC1]

In some embodiments R⁶ is selected from the group consisting of CO₂CH₃, —CH₂OCH₃, —CH₂S(O)₂ethyl, phenyl,

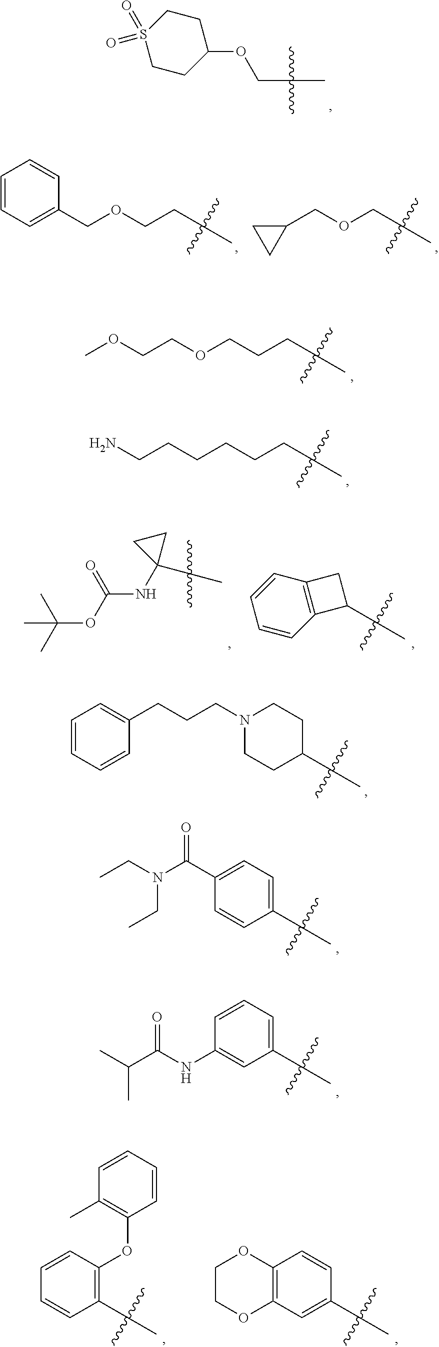

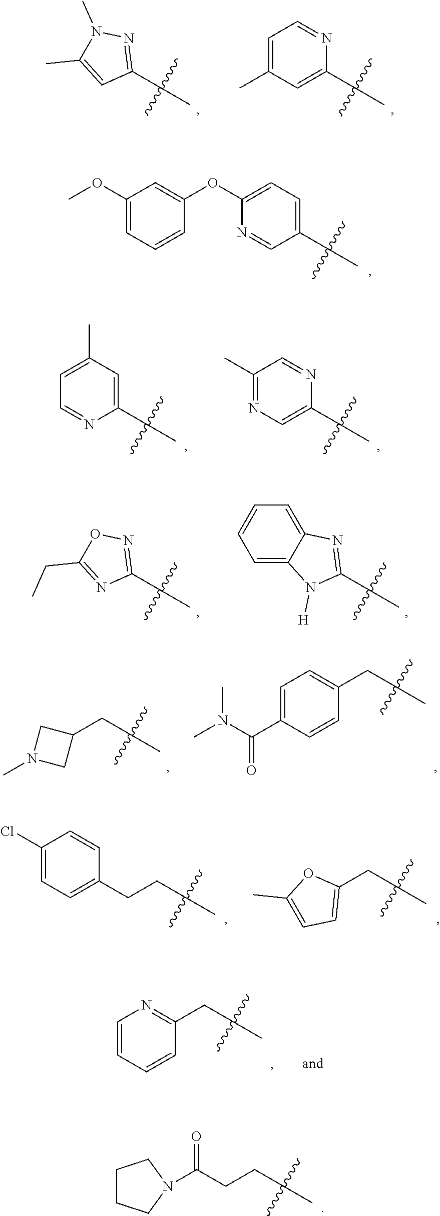

Substituent R⁷ of Formula II is selected from the group consisting H, C₁-C₆ alkyl, C₃-C₇ cycloalkyl, and ((dialkylamino)carbonyl)C₁-C₆ alkyl. In some embodiments R⁷ is selected from the group consisting H, ethyl, isobutyl, cyclopropyl, and ((dimethylamino)carbonyl)methyl.

Alternatively R⁶ and R⁷ of Formula II together with the carbon atom to which they are attached form a group selected from an optionally substituted 4 to 7-membered heterocyclyl and optionally substituted indanyl. In some embodiments, R⁶ and R⁷ together with the carbon atom to which they are attached form a group selected from optionally substituted oxanyl, optionally substituted piperidinyl, optionally substituted piperidin-2-on-yl, optionally substituted azapan-2-on-yl, optionally substituted azetidinyl and optionally substituted indanyl. In some embodiments, R⁶ and R⁷ together with the carbon atom to which they are attached form a group selected from

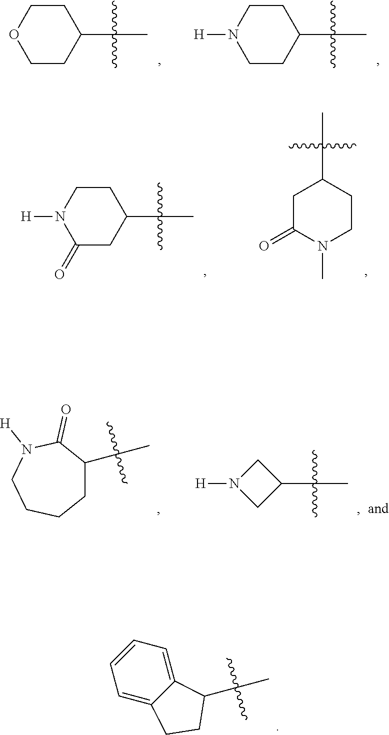

Some embodiments describe a compound of Formula II wherein
R¹ is chloro;
R² is selected from the group consisting of H and chloro;
R³ is selected from the group consisting H and F;
R⁵ is selected from the group consisting of H and C₁-C₃ alkyl;
R⁶ is selected from the group consisting of CO₂CH₃, —CH₂OCH₃, —CH₂S(O)₂ethyl, phenyl,

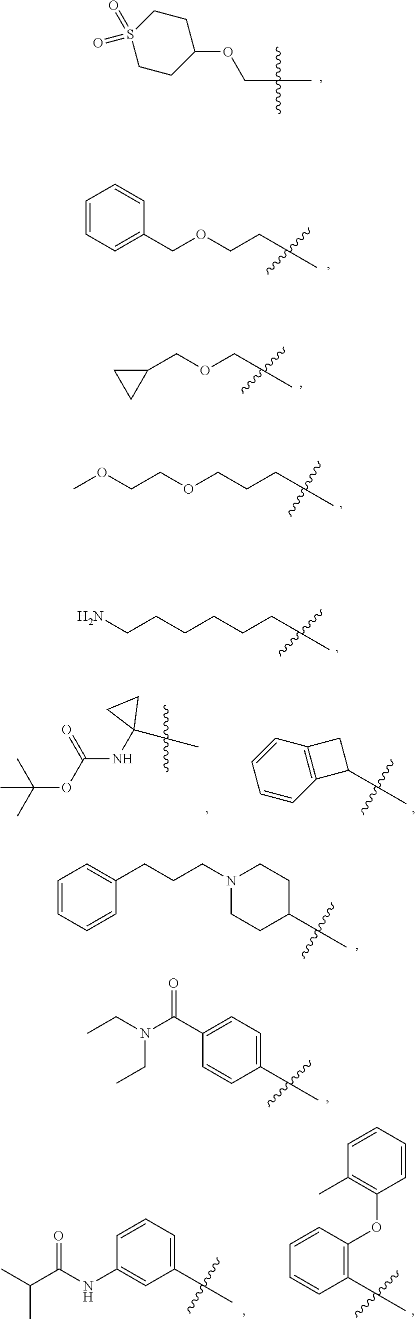

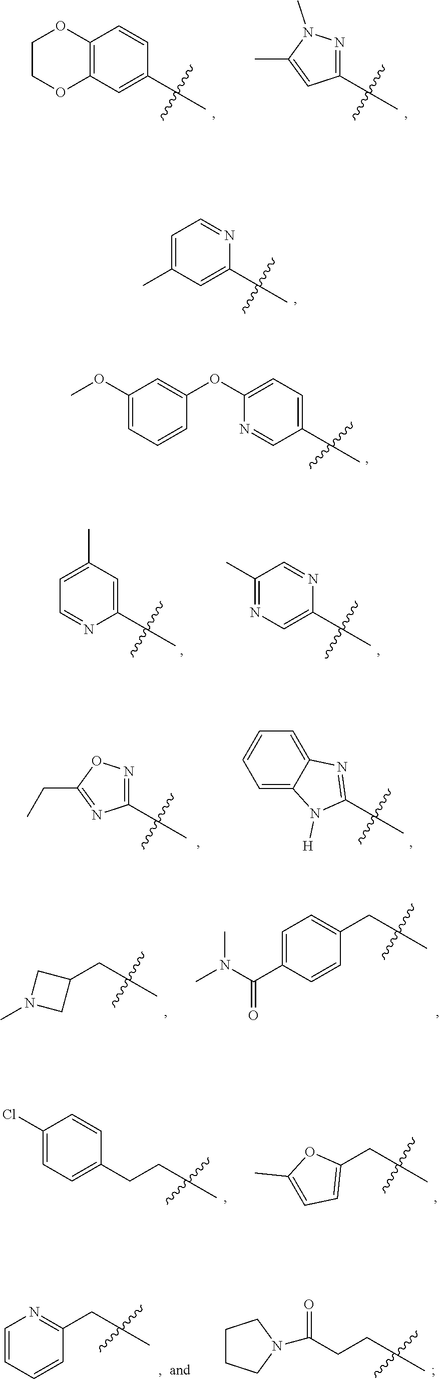

R⁷ is selected from the group consisting H, ethyl, isobutyl, cyclopropyl, and ((dimethylamino)carbonyl)methyl; or
alternatively R⁶ and R⁷ together with the carbon atom to which they are attached form a group selected from

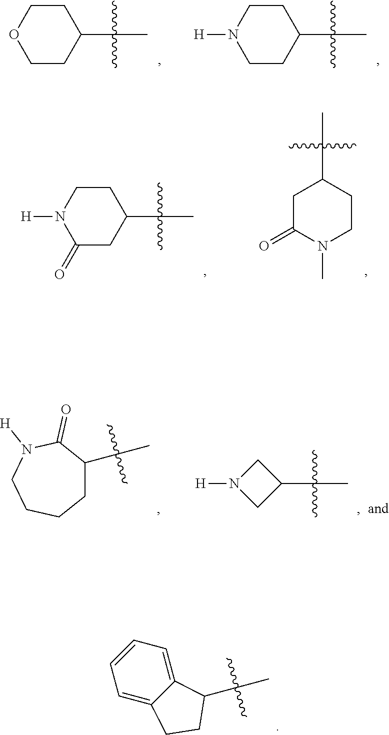

In some embodiments, the compound of Formula II is a compound of Formula IIa or Formula IIb:

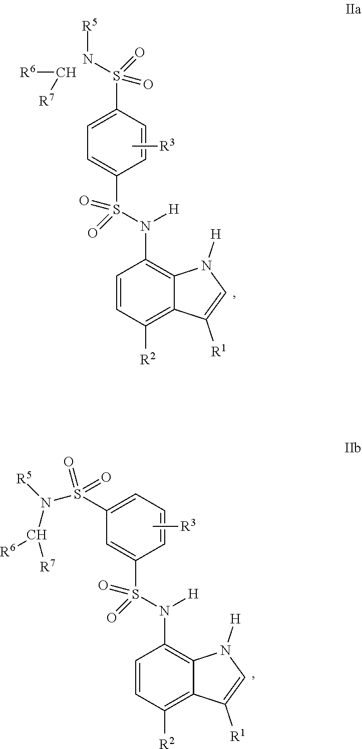

wherein R¹, R² R³, R⁵, R⁶ and R⁷ are as described for the compounds of Formula II.

In some embodiments a compound of Formula II is a compound selected

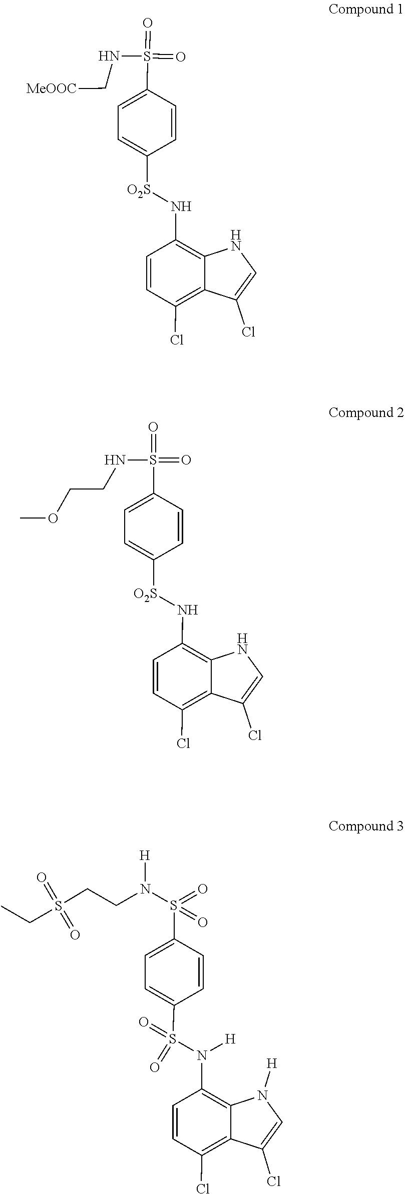 [SMILES: COC(=O)CNS(=O)(=O)c1ccc(S(=O)(=O)Nc2ccc(Cl)c3c(Cl)c[nH]c23)cc1 ; COCCNS(=O)(=O)c1ccc(S(=O)(=O)Nc2ccc(Cl)c3c(Cl)c[nH]c23)cc1 ; CCS(=O)(=O)CCNS(=O)(=O)c1ccc(S(=O)(=O)Nc2ccc(Cl)c3c(Cl)c[nH]c23)cc1]

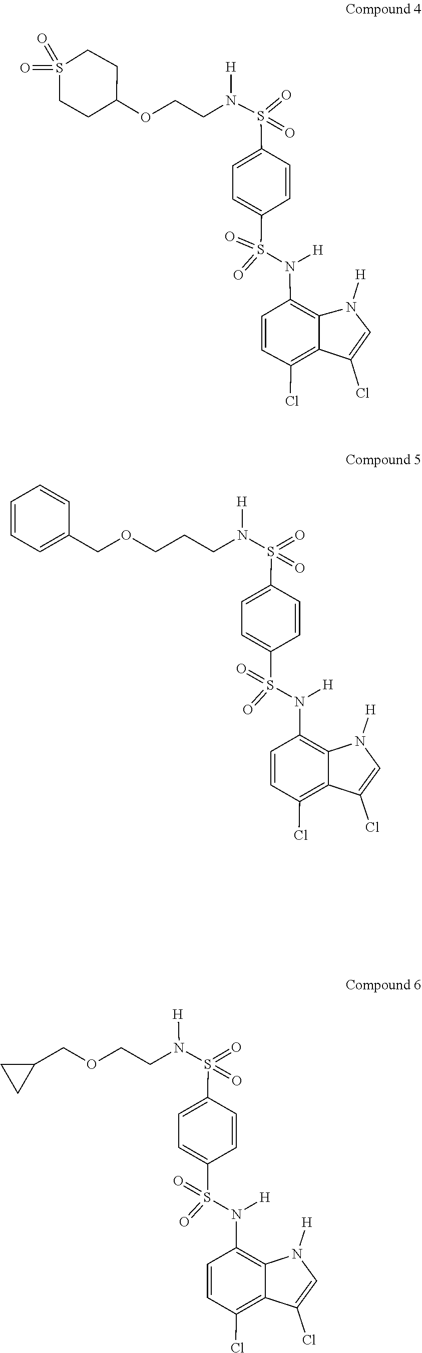 [SMILES: O=S1(=O)CCC(OCCNS(=O)(=O)c2ccc(S(=O)(=O)Nc3ccc(Cl)c4c(Cl)c[nH]c34)cc2)CC1 ; O=S(=O)(NCCCOCc1ccccc1)c1ccc(S(=O)(=O)Nc2ccc(Cl)c3c(Cl)c[nH]c23)cc1 ; O=S(=O)(NCCOCC1CC1)c1ccc(S(=O)(=O)Nc2ccc(Cl)c3c(Cl)c[nH]c23)cc1]

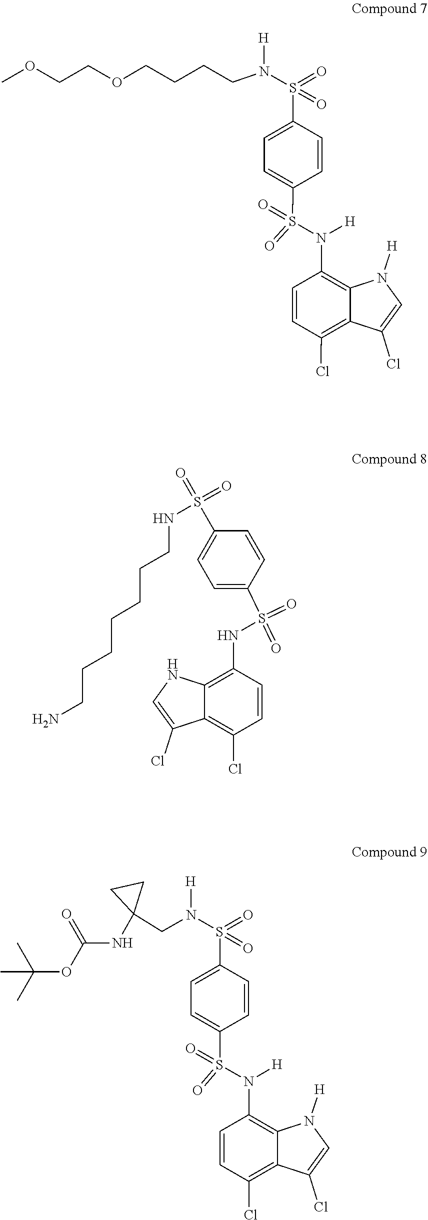 [SMILES: COCCOCCCCNS(=O)(=O)c1ccc(S(=O)(=O)Nc2ccc(Cl)c3c(Cl)c[nH]c23)cc1 ; NCCCCCCCNS(=O)(=O)c1ccc(S(=O)(=O)Nc2ccc(Cl)c3c(Cl)c[nH]c23)cc1 ; CC(C)(C)OC(=O)NC1(CNS(=O)(=O)c2ccc(S(=O)(=O)Nc3ccc(Cl)c4c(Cl)c[nH]c34)cc2)CC1]

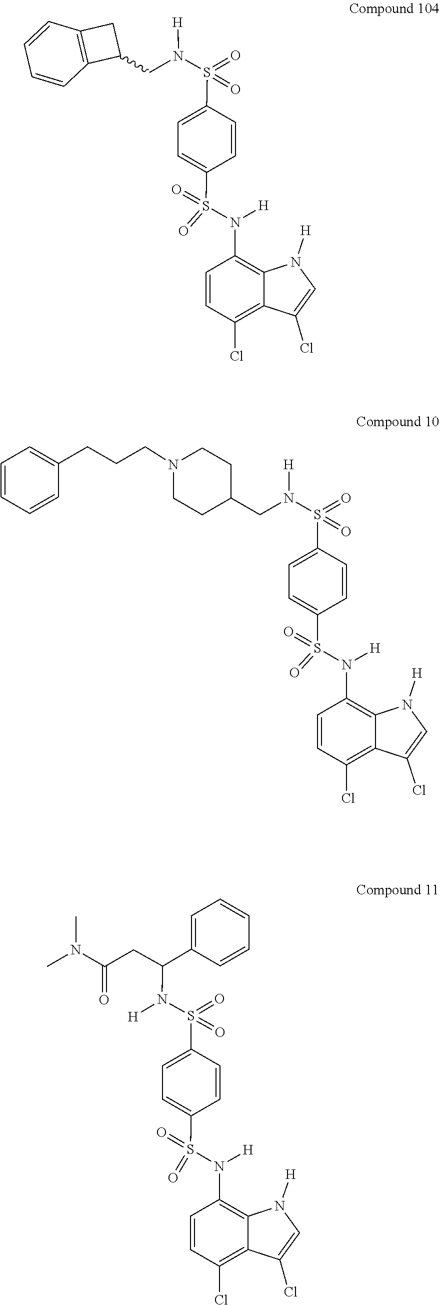 [SMILES: O=S(=O)(NCC1Cc2ccccc21)c1ccc(S(=O)(=O)Nc2ccc(Cl)c3c(Cl)c[nH]c23)cc1 ; O=S(=O)(NCC1CCN(CCCc2ccccc2)CC1)c1ccc(S(=O)(=O)Nc2ccc(Cl)c3c(Cl)c[nH]c23)cc1 ; CN(C)C(=O)CC(NS(=O)(=O)c1ccc(S(=O)(=O)Nc2ccc(Cl)c3c(Cl)c[nH]c23)cc1)c1ccccc1]

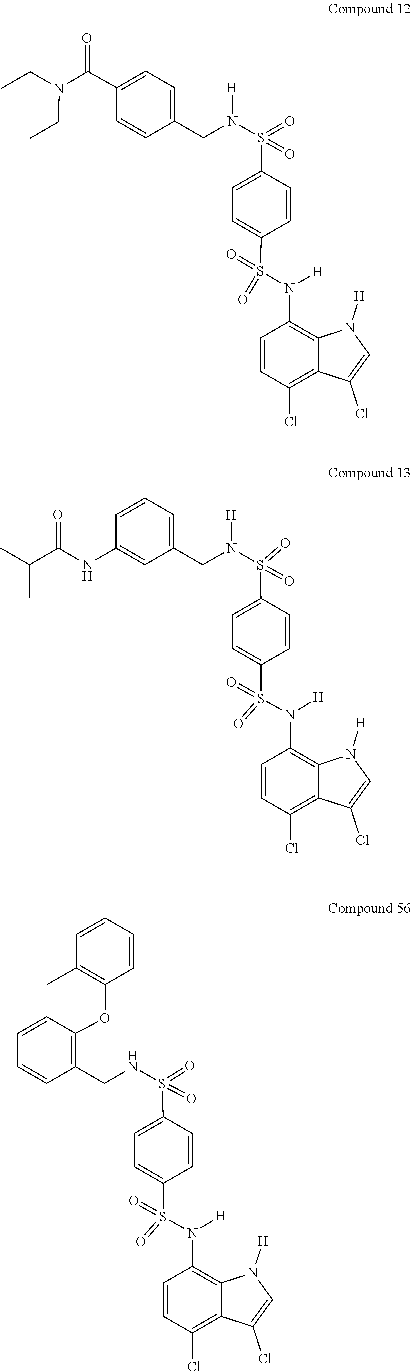 [SMILES: CCN(CC)C(=O)c1ccc(CNS(=O)(=O)c2ccc(S(=O)(=O)Nc3ccc(Cl)c4c(Cl)c[nH]c34)cc2)cc1 ; CC(C)C(=O)Nc1cccc(CNS(=O)(=O)c2ccc(S(=O)(=O)Nc3ccc(Cl)c4c(Cl)c[nH]c34)cc2)c1 ; Cc1ccccc1Oc1ccccc1CNS(=O)(=O)c1ccc(S(=O)(=O)Nc2ccc(Cl)c3c(Cl)c[nH]c23)cc1]

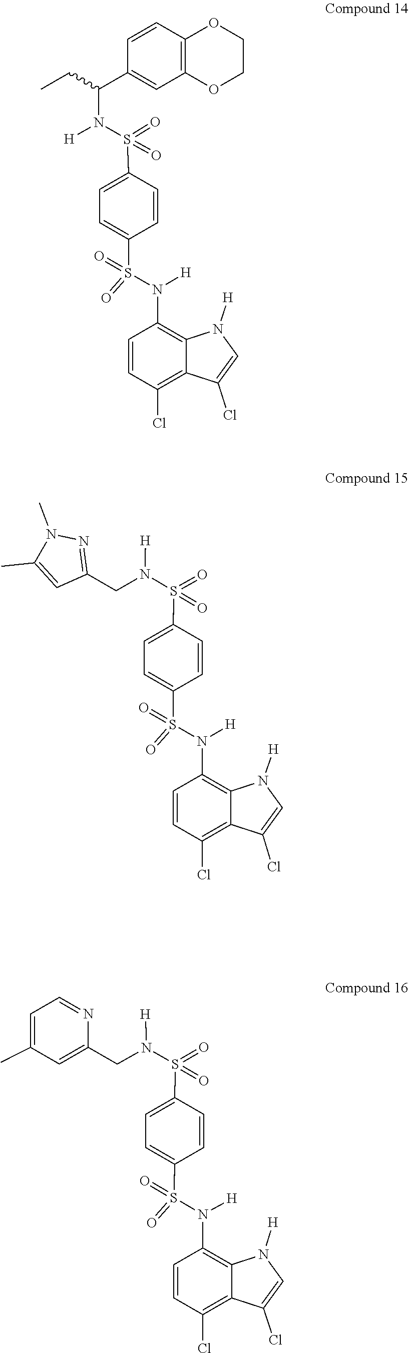 [SMILES: CCC(NS(=O)(=O)c1ccc(S(=O)(=O)Nc2ccc(Cl)c3c(Cl)c[nH]c23)cc1)c1ccc2c(c1)OCCO2 ; Cc1cc(CNS(=O)(=O)c2ccc(S(=O)(=O)Nc3ccc(Cl)c4c(Cl)c[nH]c34)cc2)nn1C ; Cc1ccnc(CNS(=O)(=O)c2ccc(S(=O)(=O)Nc3ccc(Cl)c4c(Cl)c[nH]c34)cc2)c1]

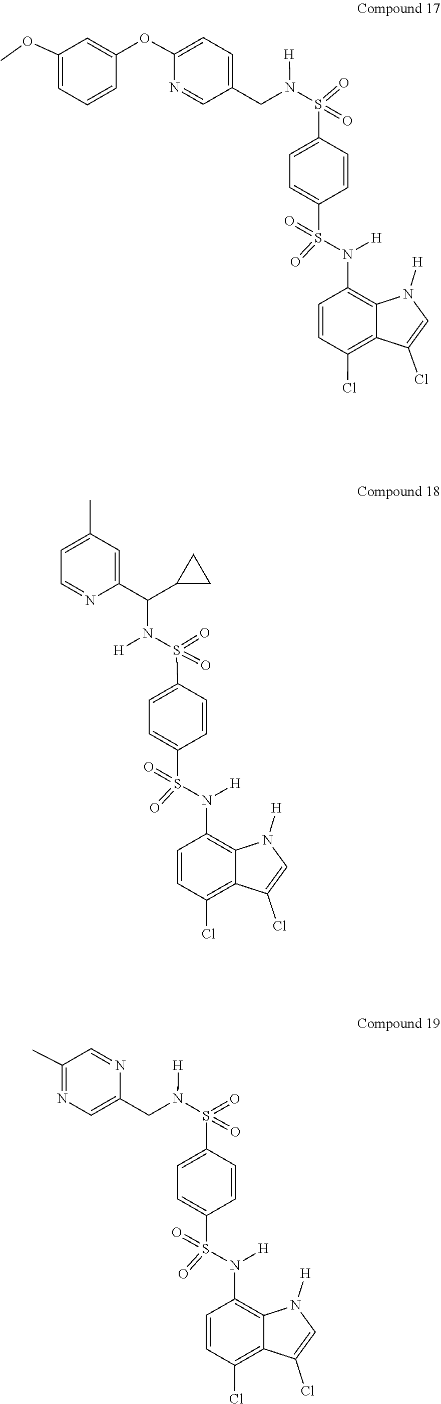 [SMILES: COc1cccc(Oc2ccc(CNS(=O)(=O)c3ccc(S(=O)(=O)Nc4ccc(Cl)c5c(Cl)c[nH]c45)cc3)cn2)c1 ; Cc1ccnc(C(NS(=O)(=O)c2ccc(S(=O)(=O)Nc3ccc(Cl)c4c(Cl)c[nH]c34)cc2)C2CC2)c1 ; Cc1cnc(CNS(=O)(=O)c2ccc(S(=O)(=O)Nc3ccc(Cl)c4c(Cl)c[nH]c34)cc2)cn1]

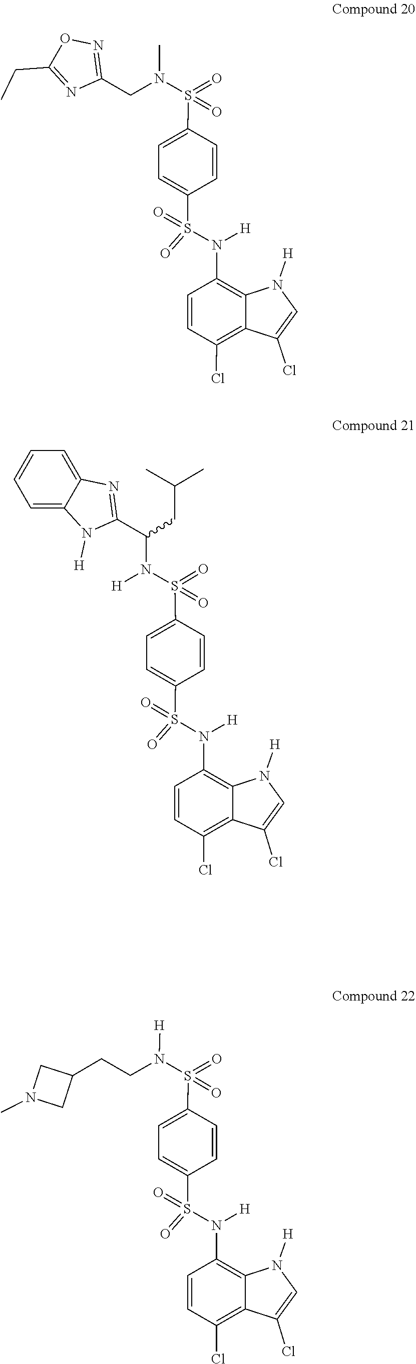 [SMILES: CCc1nc(CN(C)S(=O)(=O)c2ccc(S(=O)(=O)Nc3ccc(Cl)c4c(Cl)c[nH]c34)cc2)no1 ; CC(C)CC(NS(=O)(=O)c1ccc(S(=O)(=O)Nc2ccc(Cl)c3c(Cl)c[nH]c23)cc1)c1nc2ccccc2[nH]1 ; CN1CC(CCNS(=O)(=O)c2ccc(S(=O)(=O)Nc3ccc(Cl)c4c(Cl)c[nH]c34)cc2)C1]

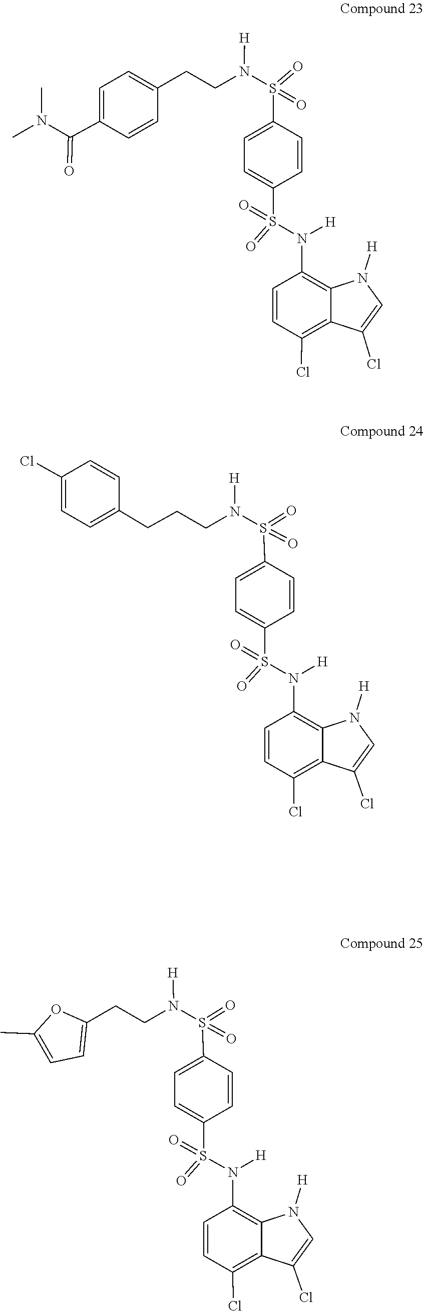 [SMILES: CN(C)C(=O)c1ccc(CCNS(=O)(=O)c2ccc(S(=O)(=O)Nc3ccc(Cl)c4c(Cl)c[nH]c34)cc2)cc1 ; O=S(=O)(NCCCc1ccc(Cl)cc1)c1ccc(S(=O)(=O)Nc2ccc(Cl)c3c(Cl)c[nH]c23)cc1 ; Cc1ccc(CCNS(=O)(=O)c2ccc(S(=O)(=O)Nc3ccc(Cl)c4c(Cl)c[nH]c34)cc2)o1]

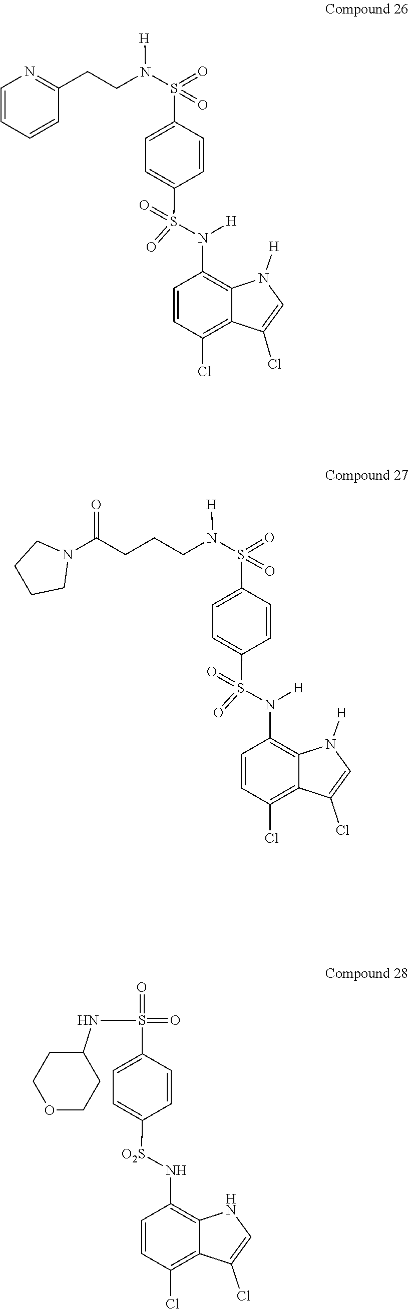 [SMILES: O=S(=O)(NCCc1ccccn1)c1ccc(S(=O)(=O)Nc2ccc(Cl)c3c(Cl)c[nH]c23)cc1 ; O=C(CCCNS(=O)(=O)c1ccc(S(=O)(=O)Nc2ccc(Cl)c3c(Cl)c[nH]c23)cc1)N1CCCC1 ; O=S(=O)(Nc1ccc(Cl)c2c(Cl)c[nH]c12)c1ccc(S(=O)(=O)NC2CCOCC2)cc1]

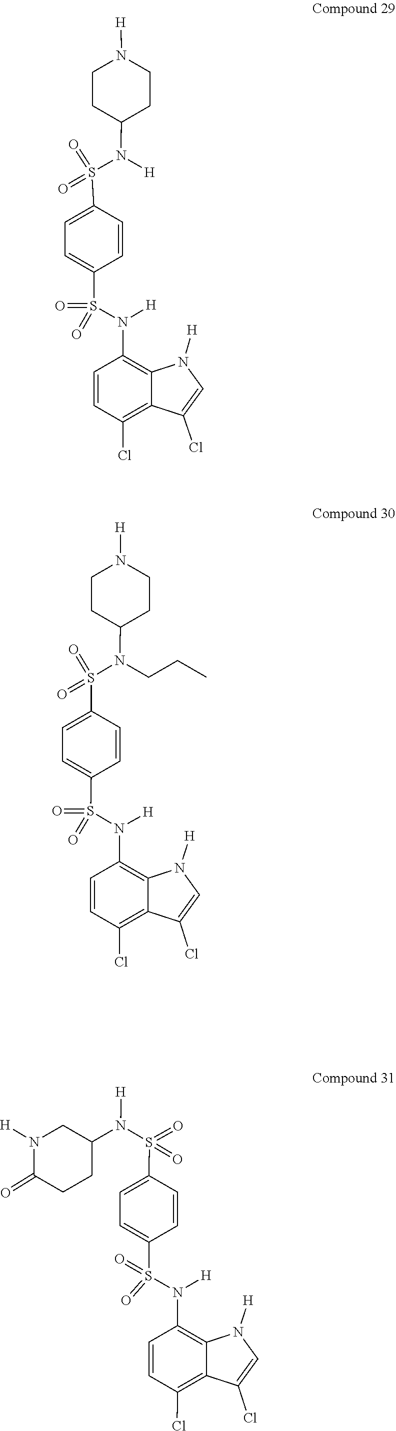 [SMILES: O=S(=O)(Nc1ccc(Cl)c2c(Cl)c[nH]c12)c1ccc(S(=O)(=O)NC2CCNCC2)cc1 ; CCCN(C1CCNCC1)S(=O)(=O)c1ccc(S(=O)(=O)Nc2ccc(Cl)c3c(Cl)c[nH]c23)cc1 ; O=C1CCC(NS(=O)(=O)c2ccc(S(=O)(=O)Nc3ccc(Cl)c4c(Cl)c[nH]c34)cc2)CN1]

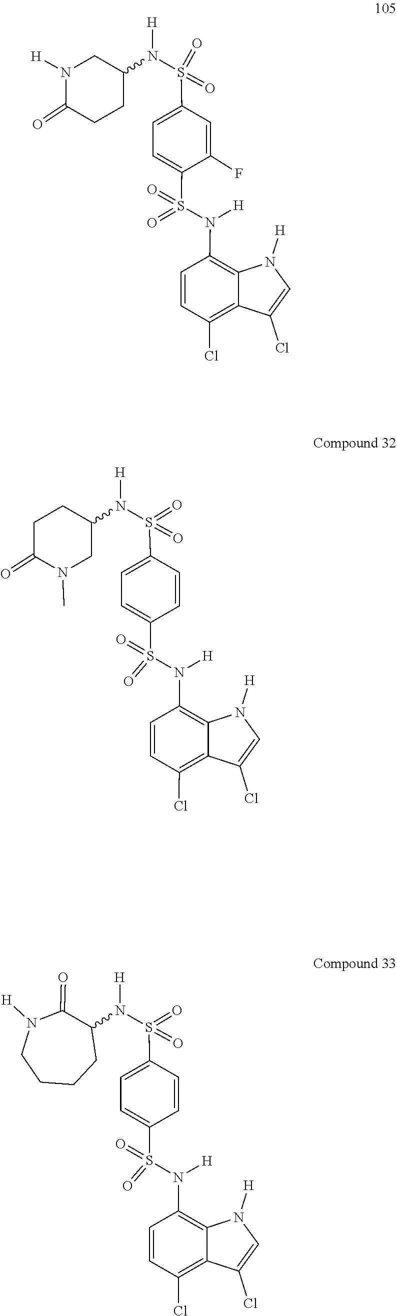 [SMILES: O=C1CCC(NS(=O)(=O)c2ccc(S(=O)(=O)Nc3ccc(Cl)c4c(Cl)c[nH]c34)c(F)c2)CN1 ; CN1CC(NS(=O)(=O)c2ccc(S(=O)(=O)Nc3ccc(Cl)c4c(Cl)c[nH]c34)cc2)CCC1=O ; O=C1NCCCCC1NS(=O)(=O)c1ccc(S(=O)(=O)Nc2ccc(Cl)c3c(Cl)c[nH]c23)cc1]

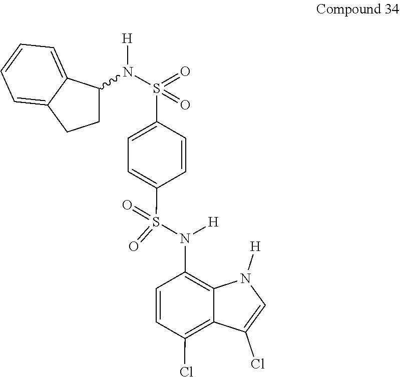 [SMILES: O=S(=O)(Nc1ccc(Cl)c2c(Cl)c[nH]c12)c1ccc(S(=O)(=O)NC2CCc3ccccc32)cc1]

or a pharmaceutically acceptable salt thereof.

In some embodiments the compound of Formula I is a compound of Formula IIc, Formula IId or Formula IIe:

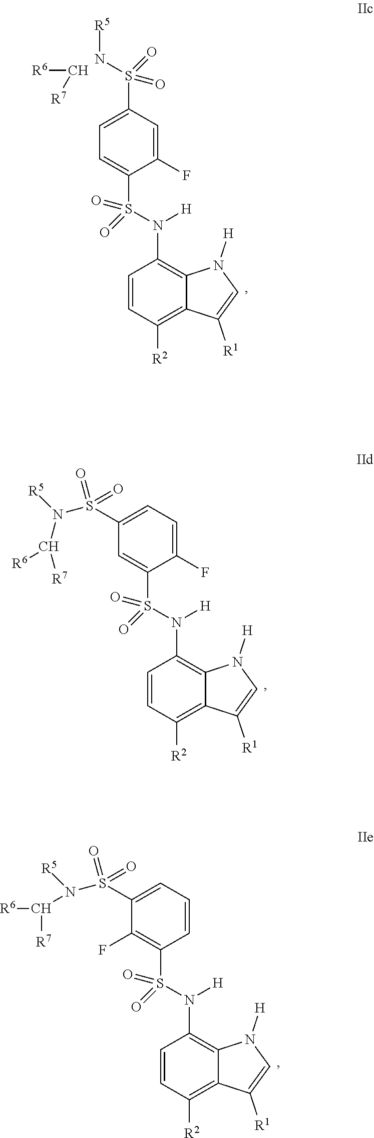

or a pharmaceutically acceptable salt thereof, wherein R¹, R², R⁵, R⁶, and R⁷, are as described according to any embodiment of compounds of Formula II.

### Compounds of Formula III

In some embodiments the compound of Formula I is a compound of Formula III

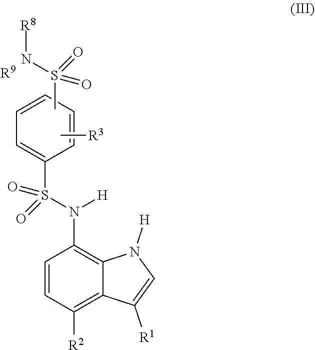

or a pharmaceutically acceptable salt thereof.

Substituent R¹ of Formula III is selected from the group consisting of chloro, bromo, fluoro, and iodo. In some embodiments, R¹ is selected from the group consisting of chloro, bromo, and iodo. In some embodiments, R¹ is chloro.

Substituent R² of Formula III is selected from the group consisting of H, chloro, fluoro, and methyl. In some embodiments R² of Formula II is selected from the group consisting of H and chloro.

Substituent R³ of Formula III is selected from the group consisting of H, chloro, fluoro, cyano and methyl. In some embodiments R³ is selected from the group consisting H, fluoro and cyano. In some embodiments, R³ is H. In some embodiments, R³ is F.

Substituents R⁸ and R⁹ together with the nitrogen atom to which they are attached form a group selected from optionally substituted 4 to 12-membered heterocyclic ring, or optionally substituted 5 to 6-membered heteroaryl ring, wherein the heterocyclic ring is a monocyclic, fused, spirocyclic or bridged heterocyclyl and with the proviso that the compound is not

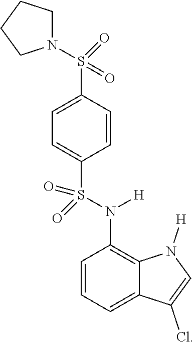 [SMILES: O=S(=O)(Nc1cccc2c(Cl)c[nH]c12)c1ccc(S(=O)(=O)N2CCCC2)cc1]

In some embodiments R⁸ and R⁹ together with the nitrogen atom to which they are attached form a group selected from optionally substituted 4 to 7-membered monocyclic heterocyclyl, optionally substituted 8 to 9-membered fused heterocyclyl, optionally substituted 7 to 11-membered spiro heterocyclyl, optionally substituted 8-membered bridged heterocyclyl, and optionally substituted 5-membered heteroaryl. In some embodiments R⁸ and R⁹ together with the nitrogen atom to which they are attached form a group selected from optionally substituted azetidinyl, optionally substituted pyrrolidinyl, optionally substituted piperidinyl, optionally substituted piperazinyl, optionally substituted 1,4-diazacyclohexyl, optionally substituted piperazin-2-one, optionally substituted morpholinyl, optionally substituted

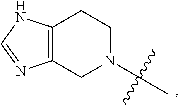 [SMILES: *N1CCc2[nH]cnc2C1]

optionally substituted

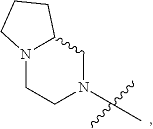 [SMILES: *N1CCN2CCCC2C1]

optionally substituted

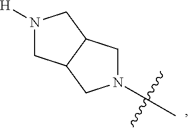 [SMILES: *N1CC2CNCC2C1]

optionally substituted

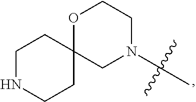 [SMILES: *N1CCOC2(CCNCC2)C1]

optionally substituted

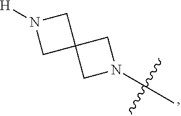 [SMILES: *N1CC2(CNC2)C1]

optionally substitute

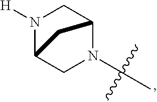 [SMILES: *N1C[C@H]2C[C@@H]1CN2]

and optionally substituted 1,2,3-triazolyl. In some embodiments R⁸ and R⁹ together with the nitrogen atom to which they are attached form a group selected from piperidinyl,

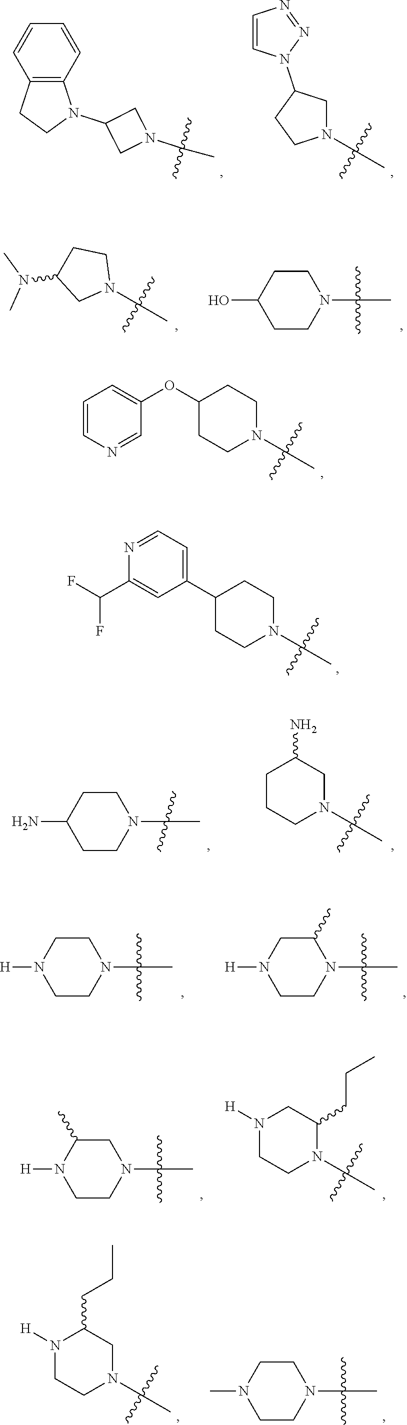

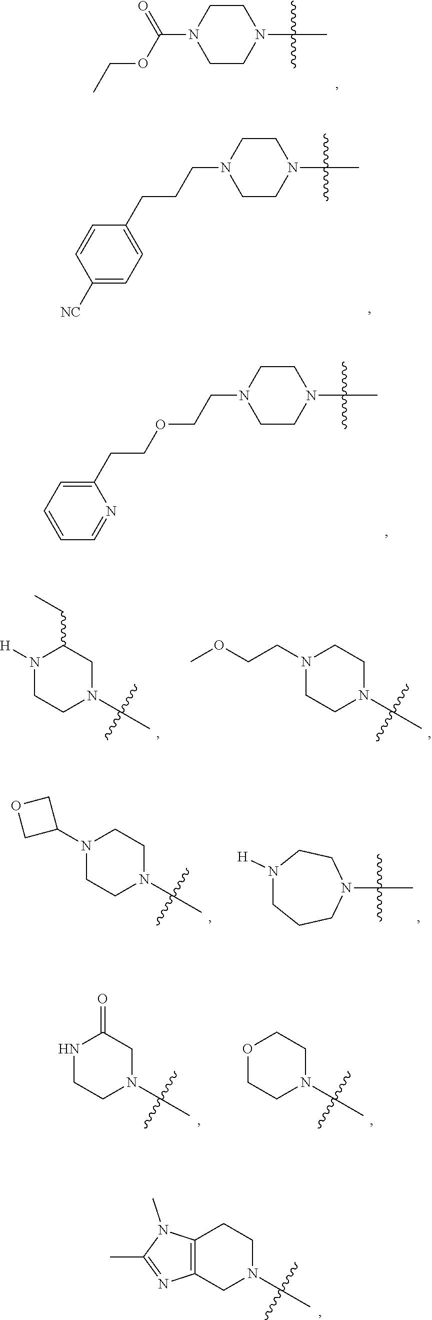

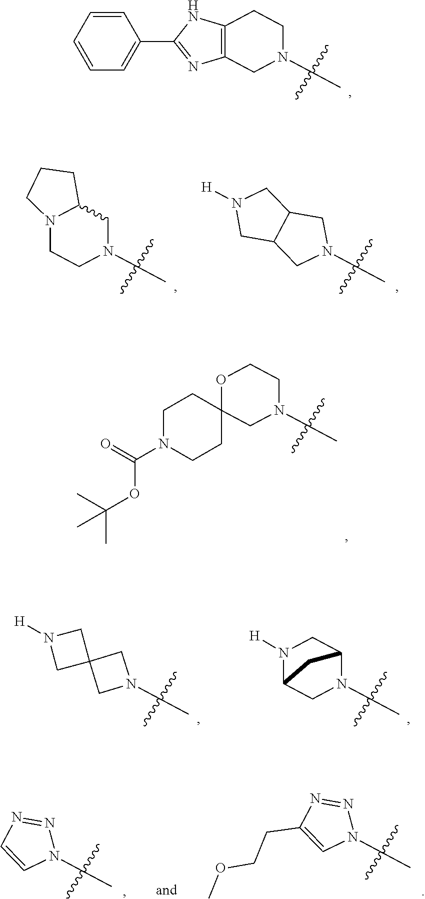

Some embodiments describe a compound of Formula III wherein
R¹ is chloro;
R² is selected from the group consisting of H and chloro;
R³ is selected from the group consisting H, fluoro, and cyano;
R⁸ and R⁹ together with the nitrogen atom to which they are attached form a group selected from a group selected from piperidinyl,

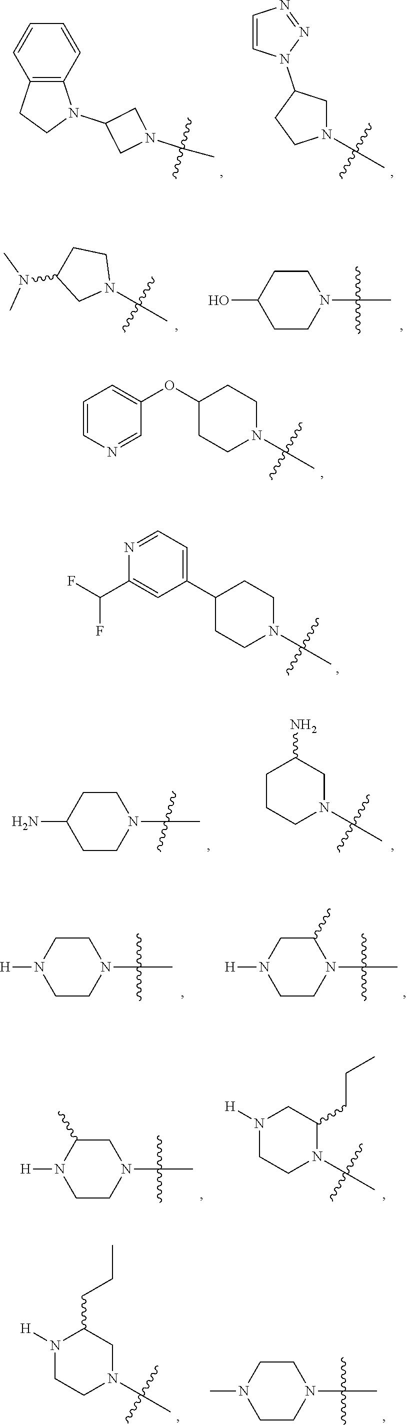

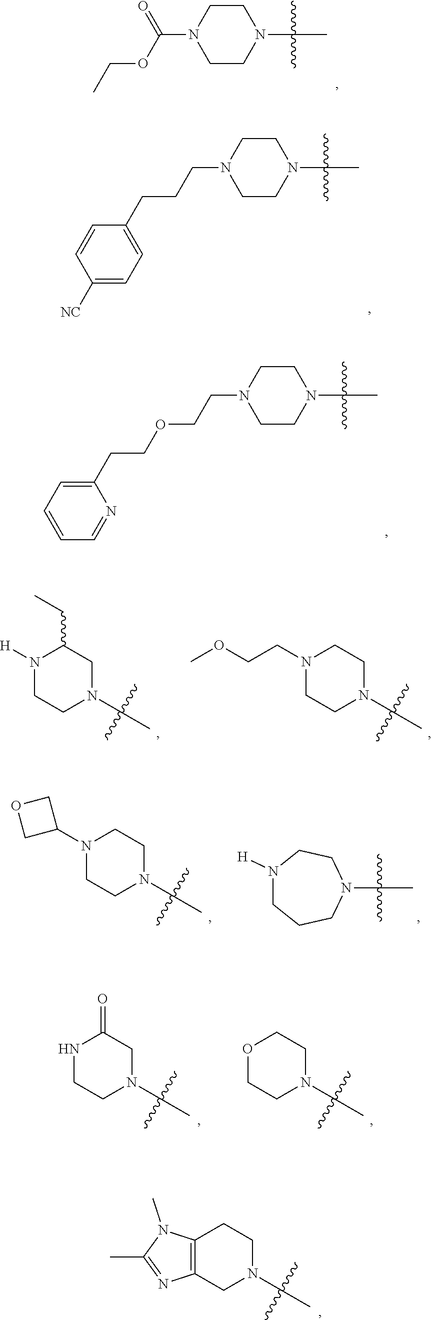

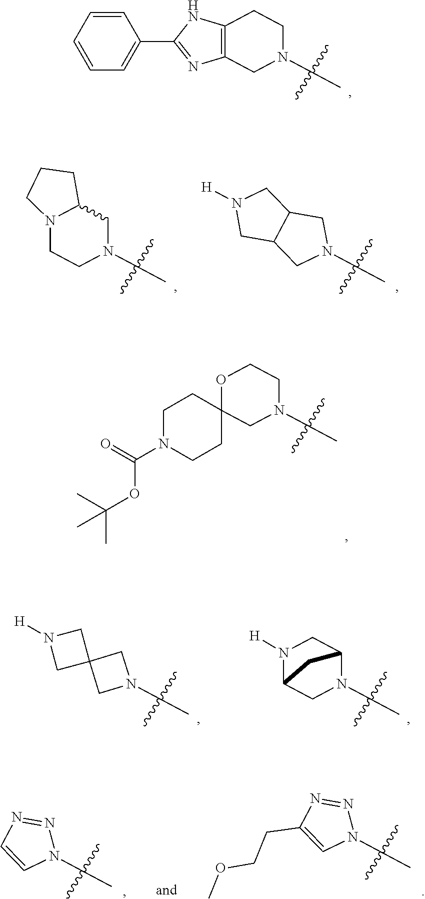

In some embodiments, the compound of Formula III is a compound of Formula IIIa or Formula IIIb:

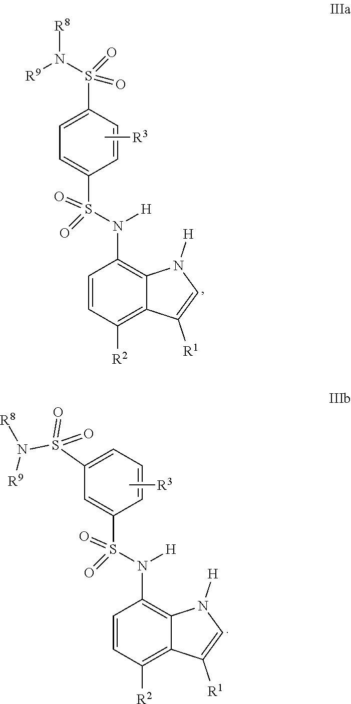

In some embodiments a compound of Formula III is selected from

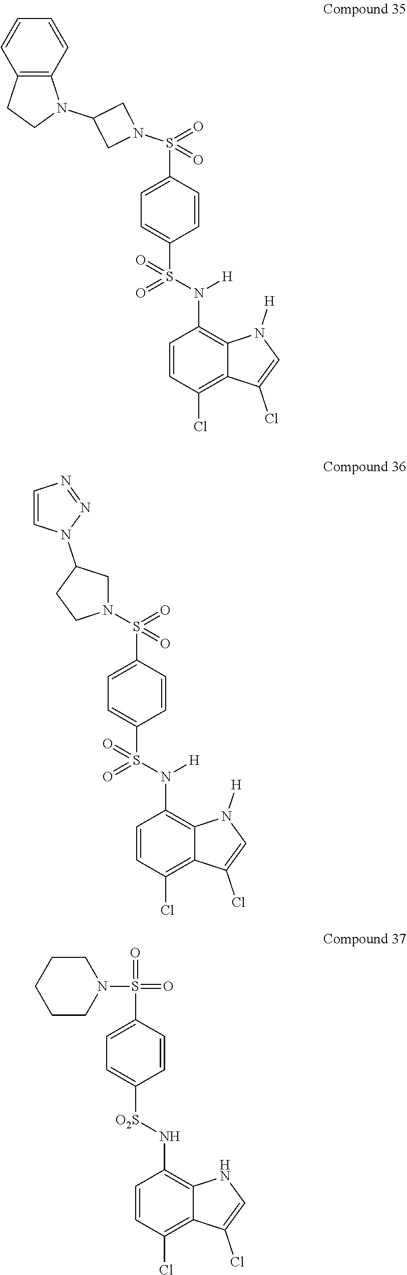 [SMILES: O=S(=O)(Nc1ccc(Cl)c2c(Cl)c[nH]c12)c1ccc(S(=O)(=O)N2CC(N3CCc4ccccc43)C2)cc1 ; O=S(=O)(Nc1ccc(Cl)c2c(Cl)c[nH]c12)c1ccc(S(=O)(=O)N2CCC(n3ccnn3)C2)cc1 ; O=S(=O)(Nc1ccc(Cl)c2c(Cl)c[nH]c12)c1ccc(S(=O)(=O)N2CCCCC2)cc1]

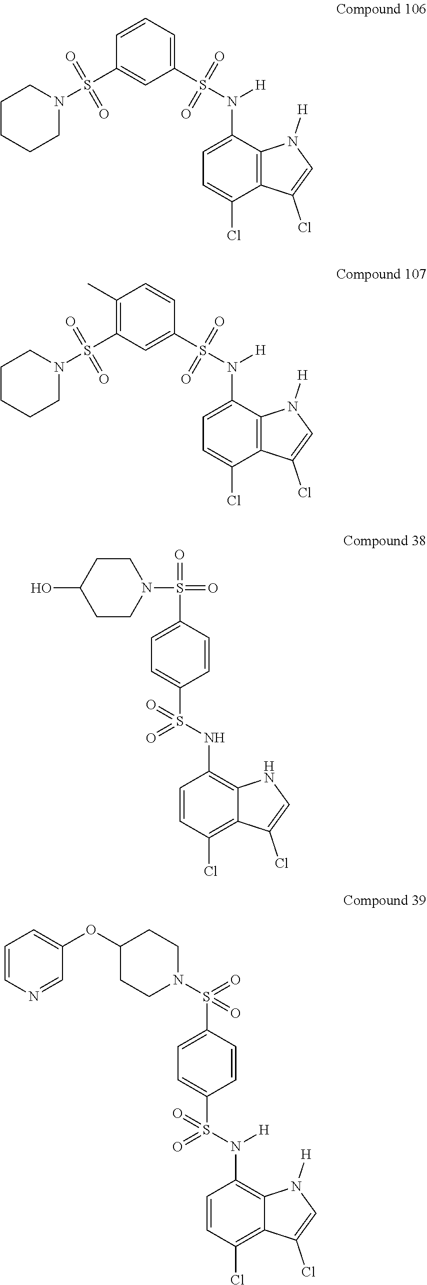 [SMILES: O=S(=O)(Nc1ccc(Cl)c2c(Cl)c[nH]c12)c1cccc(S(=O)(=O)N2CCCCC2)c1 ; Cc1ccc(S(=O)(=O)Nc2ccc(Cl)c3c(Cl)c[nH]c23)cc1S(=O)(=O)N1CCCCC1 ; O=S(=O)(Nc1ccc(Cl)c2c(Cl)c[nH]c12)c1ccc(S(=O)(=O)N2CCC(O)CC2)cc1 ; O=S(=O)(Nc1ccc(Cl)c2c(Cl)c[nH]c12)c1ccc(S(=O)(=O)N2CCC(Oc3cccnc3)CC2)cc1]

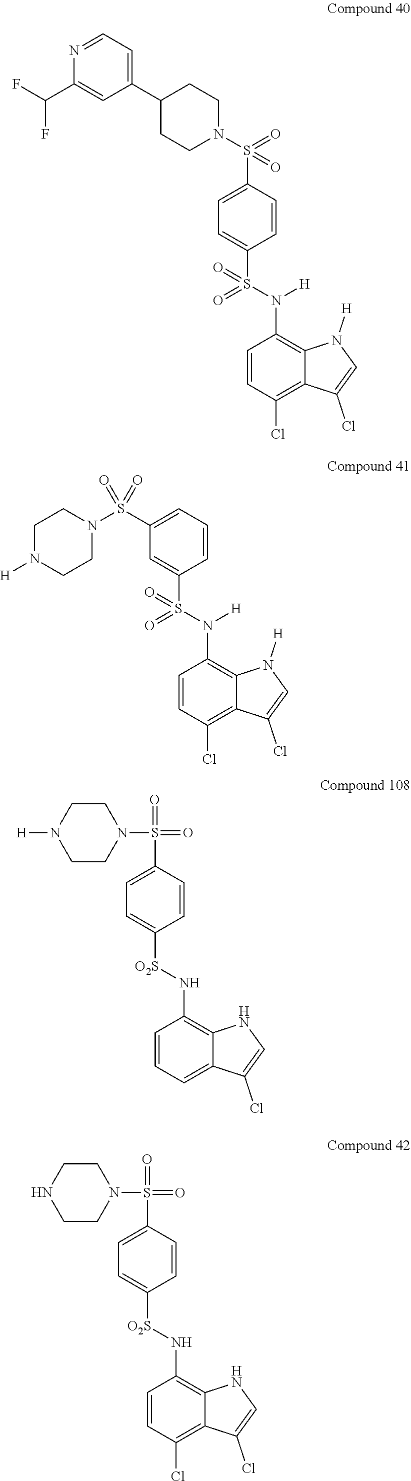 [SMILES: O=S(=O)(Nc1ccc(Cl)c2c(Cl)c[nH]c12)c1ccc(S(=O)(=O)N2CCC(c3ccnc(C(F)F)c3)CC2)cc1 ; O=S(=O)(Nc1ccc(Cl)c2c(Cl)c[nH]c12)c1cccc(S(=O)(=O)N2CCNCC2)c1 ; O=S(=O)(Nc1cccc2c(Cl)c[nH]c12)c1ccc(S(=O)(=O)N2CCNCC2)cc1 ; O=S(=O)(Nc1ccc(Cl)c2c(Cl)c[nH]c12)c1ccc(S(=O)(=O)N2CCNCC2)cc1]

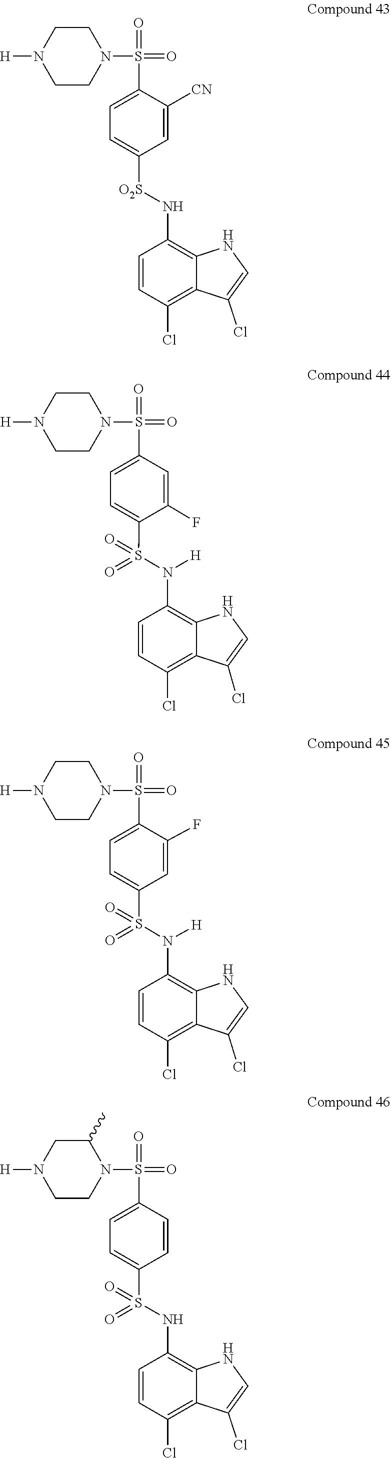 [SMILES: N#Cc1cc(S(=O)(=O)Nc2ccc(Cl)c3c(Cl)c[nH]c23)ccc1S(=O)(=O)N1CCNCC1 ; O=S(=O)(Nc1ccc(Cl)c2c(Cl)c[nH]c12)c1ccc(S(=O)(=O)N2CCNCC2)cc1F ; O=S(=O)(Nc1ccc(Cl)c2c(Cl)c[nH]c12)c1ccc(S(=O)(=O)N2CCNCC2)c(F)c1 ; CC1CNCCN1S(=O)(=O)c1ccc(S(=O)(=O)Nc2ccc(Cl)c3c(Cl)c[nH]c23)cc1]

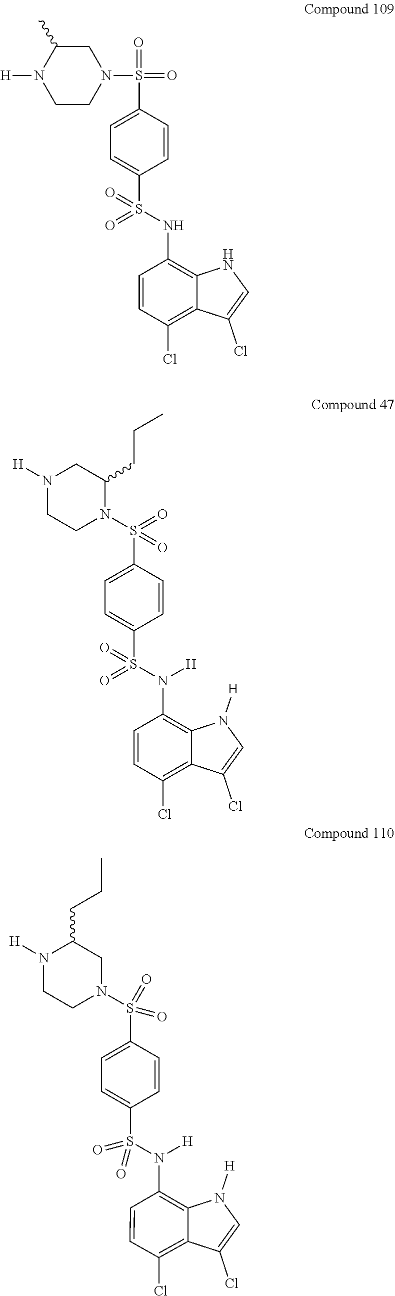 [SMILES: CC1CN(S(=O)(=O)c2ccc(S(=O)(=O)Nc3ccc(Cl)c4c(Cl)c[nH]c34)cc2)CCN1 ; CCCC1CNCCN1S(=O)(=O)c1ccc(S(=O)(=O)Nc2ccc(Cl)c3c(Cl)c[nH]c23)cc1 ; CCCC1CN(S(=O)(=O)c2ccc(S(=O)(=O)Nc3ccc(Cl)c4c(Cl)c[nH]c34)cc2)CCN1]

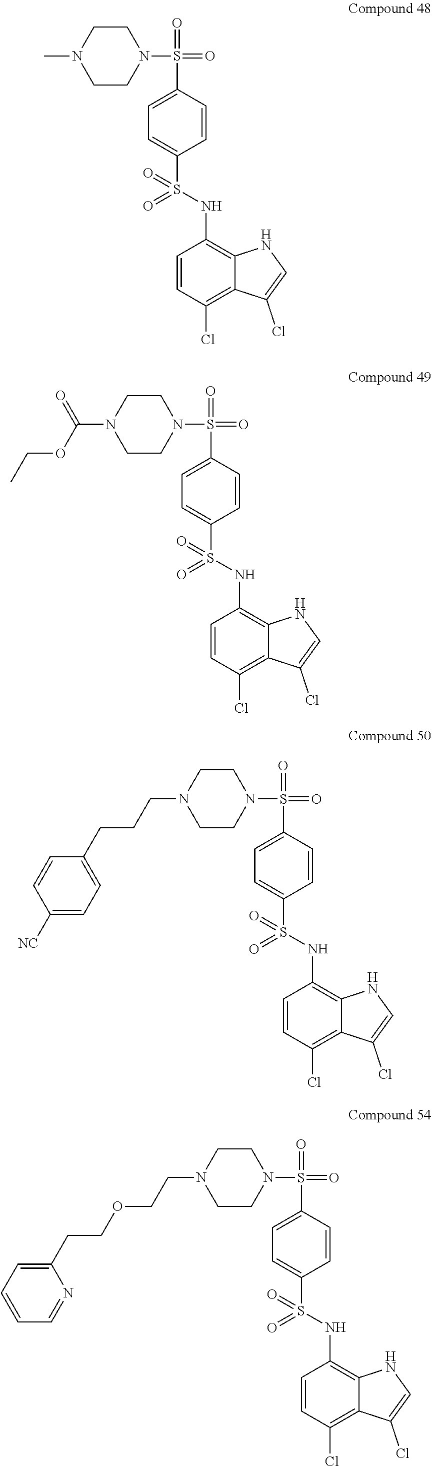 [SMILES: CN1CCN(S(=O)(=O)c2ccc(S(=O)(=O)Nc3ccc(Cl)c4c(Cl)c[nH]c34)cc2)CC1 ; CCOC(=O)N1CCN(S(=O)(=O)c2ccc(S(=O)(=O)Nc3ccc(Cl)c4c(Cl)c[nH]c34)cc2)CC1 ; N#Cc1ccc(CCCN2CCN(S(=O)(=O)c3ccc(S(=O)(=O)Nc4ccc(Cl)c5c(Cl)c[nH]c45)cc3)CC2)cc1 ; O=S(=O)(Nc1ccc(Cl)c2c(Cl)c[nH]c12)c1ccc(S(=O)(=O)N2CCN(CCOCCc3ccccn3)CC2)cc1]

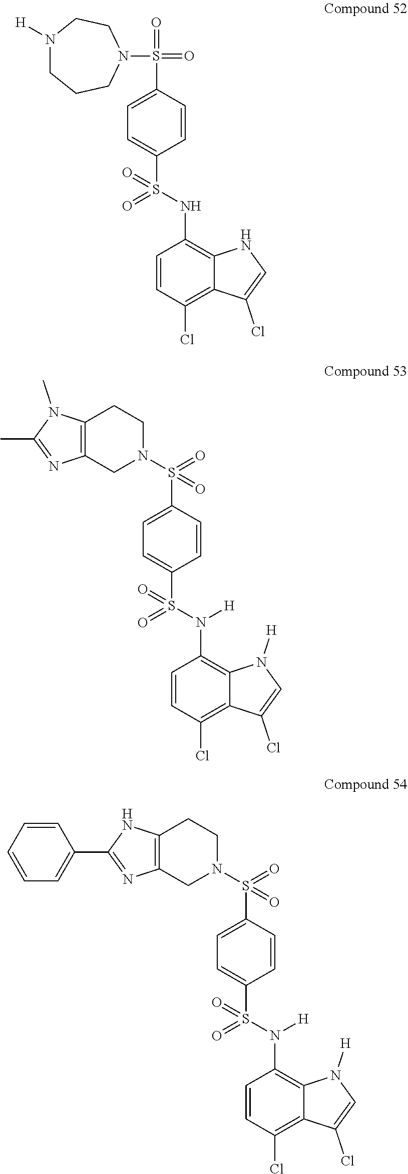 [SMILES: O=S(=O)(Nc1ccc(Cl)c2c(Cl)c[nH]c12)c1ccc(S(=O)(=O)N2CCCNCC2)cc1 ; Cc1nc2c(n1C)CCN(S(=O)(=O)c1ccc(S(=O)(=O)Nc3ccc(Cl)c4c(Cl)c[nH]c34)cc1)C2 ; O=S(=O)(Nc1ccc(Cl)c2c(Cl)c[nH]c12)c1ccc(S(=O)(=O)N2CCc3[nH]c(-c4ccccc4)nc3C2)cc1]

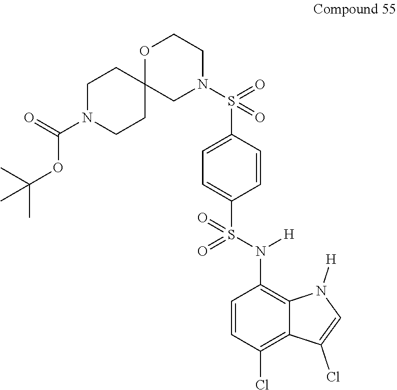 [SMILES: CC(C)(C)OC(=O)N1CCC2(CC1)CN(S(=O)(=O)c1ccc(S(=O)(=O)Nc3ccc(Cl)c4c(Cl)c[nH]c34)cc1)CCO2]

or a pharmaceutically acceptable salt thereof.

In some embodiments the compound of Formula I is a compound of Formula IIIc, Formula IIId or Formula IIIe:

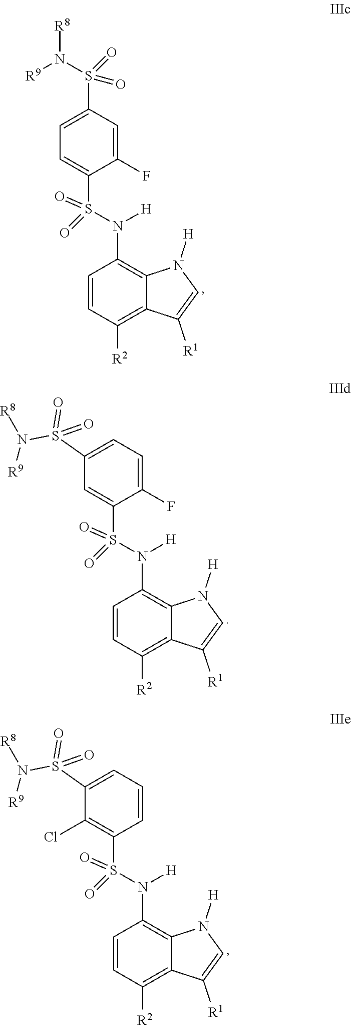

or a pharmaceutically acceptable salt thereof, wherein R¹, R², R⁸, and R⁹, are as described according to any embodiment of compounds of Formula III.

### Compounds of Formula IV

In some embodiments the compound of Formula I is a compound of Formula IV

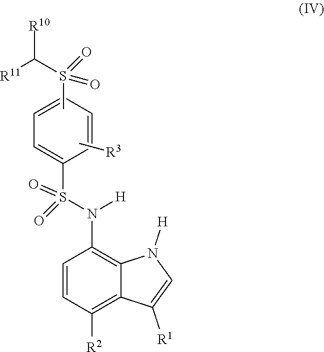

or a pharmaceutically acceptable salt thereof.

Substituent R¹ of Formula IV is selected from the group consisting of chloro, bromo, fluoro, and iodo. In some embodiments, R¹ is selected from the group consisting of chloro, bromo, and iodo. In some embodiments, R¹ is chloro.

Substituent R² of Formula IV is selected from the group consisting of H, chloro, fluoro and methyl. In some embodiments R² of Formula II is selected from the group consisting of H and chloro.

Substituent R³ of Formula IV is selected from the group consisting of H, chloro, fluoro, cyano and methyl. In some embodiments R³ is selected from the group consisting H and fluoro. In some embodiments, R³ is H. In some embodiments, R³ is F.

Substituents R¹⁰ and R¹¹ together with the carbon atom to which they are attached form an optionally substituted 6-membered heterocyclyl. In some embodiments R¹⁰ and R¹¹ together with the carbon atom to which they are attached form an optionally substituted piperidinyl. In some embodiments R¹⁰ and R¹¹ together with the carbon atom to which they are attached form

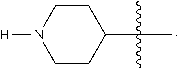 [SMILES: *C1CCNCC1]

Some embodiments describe a compound of Formula IV wherein
R¹ is chloro;
R² is selected from the group consisting of H and chloro;
R³ is selected from the group consisting H and F; and
R¹⁰ and R¹¹ together with the carbon atom to which they are attached form

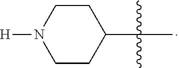 [SMILES: *C1CCNCC1]

In some embodiments, the compound of Formula IV is a compound of Formula IVa or Formula IVb:

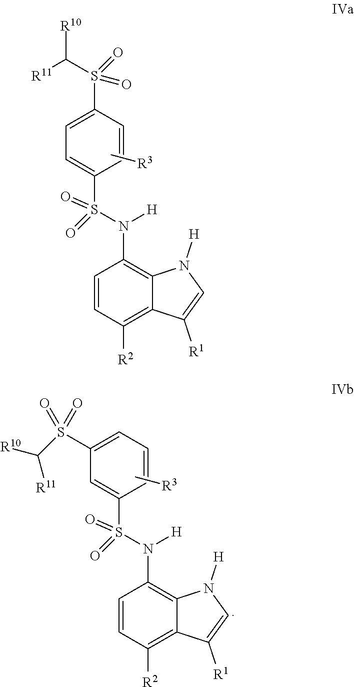

In some embodiments a compound of Formula IV is selected from

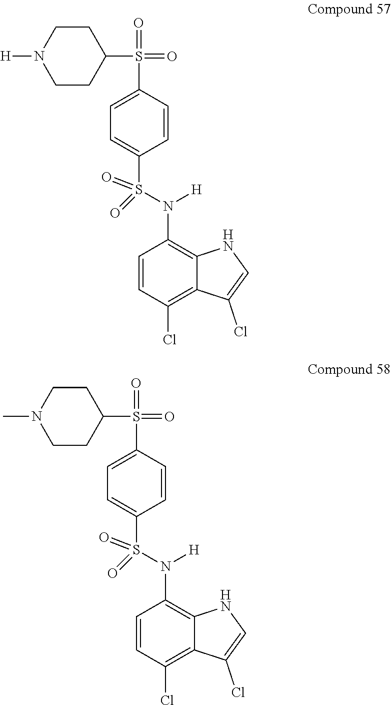 [SMILES: O=S(=O)(Nc1ccc(Cl)c2c(Cl)c[nH]c12)c1ccc(S(=O)(=O)C2CCNCC2)cc1 ; CN1CCC(S(=O)(=O)c2ccc(S(=O)(=O)Nc3ccc(Cl)c4c(Cl)c[nH]c34)cc2)CC1]

or a pharmaceutically acceptable salt thereof.

In some embodiments the compound of Formula I is a compound of Formula IVc, Formula IVd or Formula IVe:

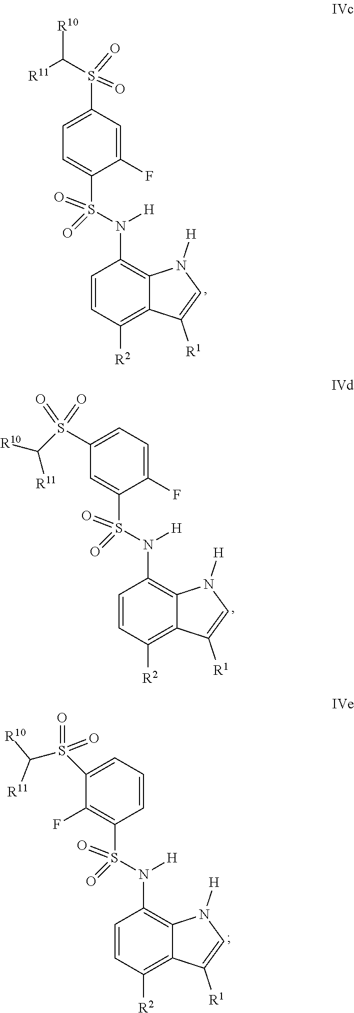

or a pharmaceutically acceptable salt thereof, wherein R¹, R², R¹⁰, and R¹¹, are as described according to any embodiment of compounds of Formula IV.

### Compounds of Formula V

In some embodiments the compound of Formula I is a compound of Formula V

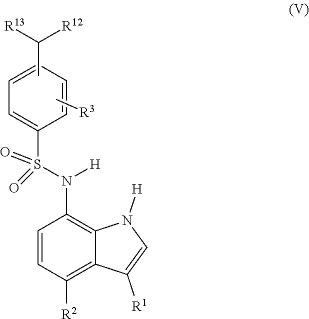

or a pharmaceutically acceptable salt thereof.

Substituent R¹ of Formula V is selected from the group consisting of chloro, bromo, fluoro, and iodo. In some embodiments, R¹ is selected from the group consisting of chloro, bromo, and iodo. In some embodiments, R¹ is chloro.

Substituent R² of Formula V is selected from the group consisting of H, chloro, fluoro and methyl. In some embodiments R² of Formula II is selected from the group consisting of H and chloro.

Substituent R³ of Formula V is selected from the group consisting of H, chloro, fluoro cyano and methyl. In some embodiments R³ is selected from the group consisting of H, chloro, fluoro and methyl. In some embodiments, R³ is H. In some embodiments, R³ is F.

Substituents R¹² of Formula V is H

Substituents R¹³ of Formula V is selected from the group consisting of —NHCOC₁-C₂ alkyl, —CH₂NHCOC₁-C₂ alkyl, —NHCO₂C₁-C₄ alkyl, —CH₂NHCO₂C₁-C₄ alkyl and optionally substituted triazole. In some embodiments R¹³ is selected from the group consisting of —NHCOCH₂CH₃, —CH₂NHCOCH₃, —NHCO₂t-Bu, —CH₂NHCO₂t-Bu, and

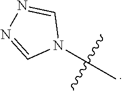 [SMILES: *n1cnnc1]

Alternatively, substituents R¹² and R¹³ together with the carbon atom to which they are attached form a group selected from optionally substituted C₃-C₆ cycloalkyl, optionally substituted 4 to 6-membered heterocyclyl, optionally substituted phenyl, and optionally substituted 5 to 6-membered-heteroaryl. In some embodiments R¹² and R¹³ together with the carbon atom to which they are attached form a group selected from optionally substituted cyclopropyl, cyclohexyl, optionally substituted oxetanyl, phenyl, optionally substituted thiophenyl, optionally substituted pyrazolyl, optionally substituted thiazolyl, optionally substituted 1,3,4-oxadiazolyl, optionally substituted optionally substituted 1,2,4-oxadiazolyl, and optionally substituted tetrazolyl. In some embodiments R¹² and R¹³ together with the carbon atom to which they are attached form a group selected from cyclohexyl, phenyl,

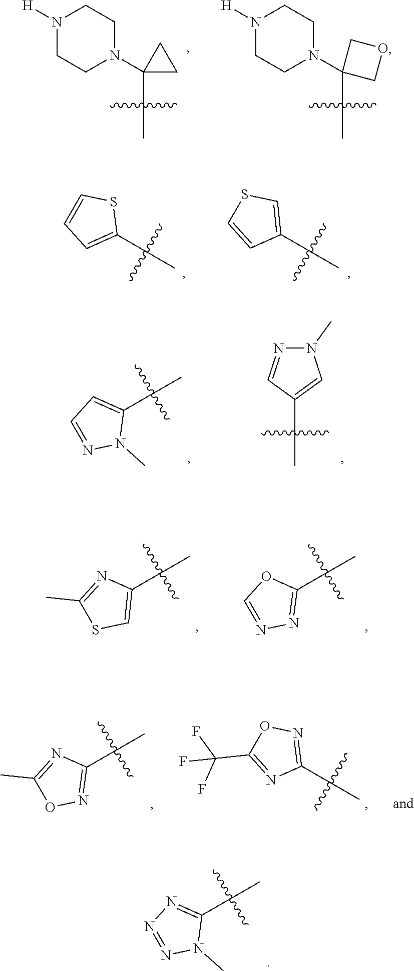

Some embodiments describe a compound of Formula V wherein
R¹ is chloro;
R² is selected from the group consisting of H and chloro;
R³ is selected from the group consisting of H, fluoro, chloro and methyl;
R¹² is H; and
R¹³ is selected from the group consisting of —NHCOCH₂CH₃, —CH₂NHCOCH₃, —NHCO₂t-Bu, —CH₂NHCO₂t-Bu, and

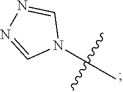 [SMILES: *n1cnnc1]

or
alternatively, R¹² and R¹³ together with the carbon atom to which they are attached form a group selected from cyclohexyl, phenyl,

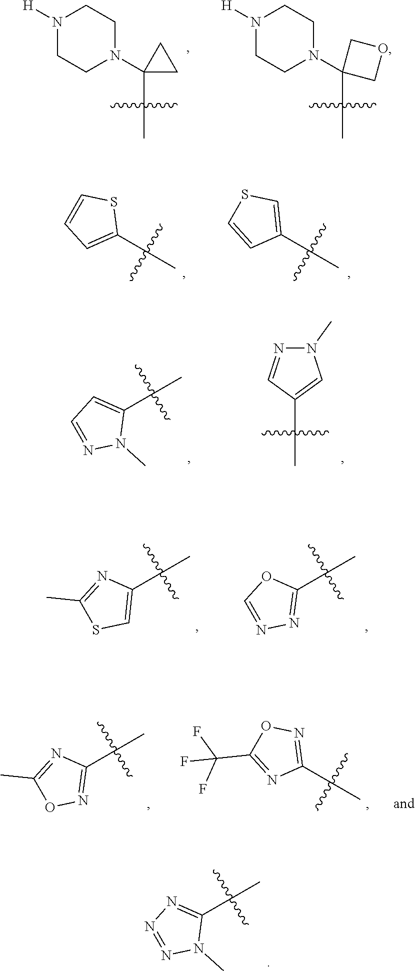

In some embodiments, the compound of Formula V is a compound of Formula Va or Formula Vb:

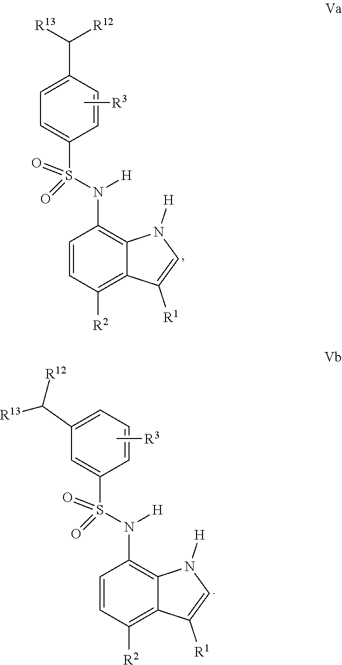

In some embodiments a compound of Formula V is selected from

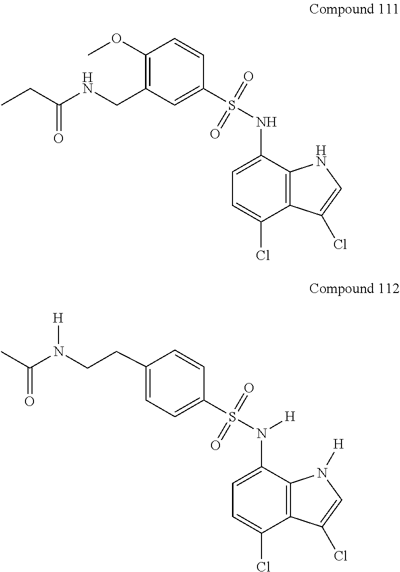 [SMILES: CCC(=O)NCc1cc(S(=O)(=O)Nc2ccc(Cl)c3c(Cl)c[nH]c23)ccc1OC ; CC(=O)NCCc1ccc(S(=O)(=O)Nc2ccc(Cl)c3c(Cl)c[nH]c23)cc1]

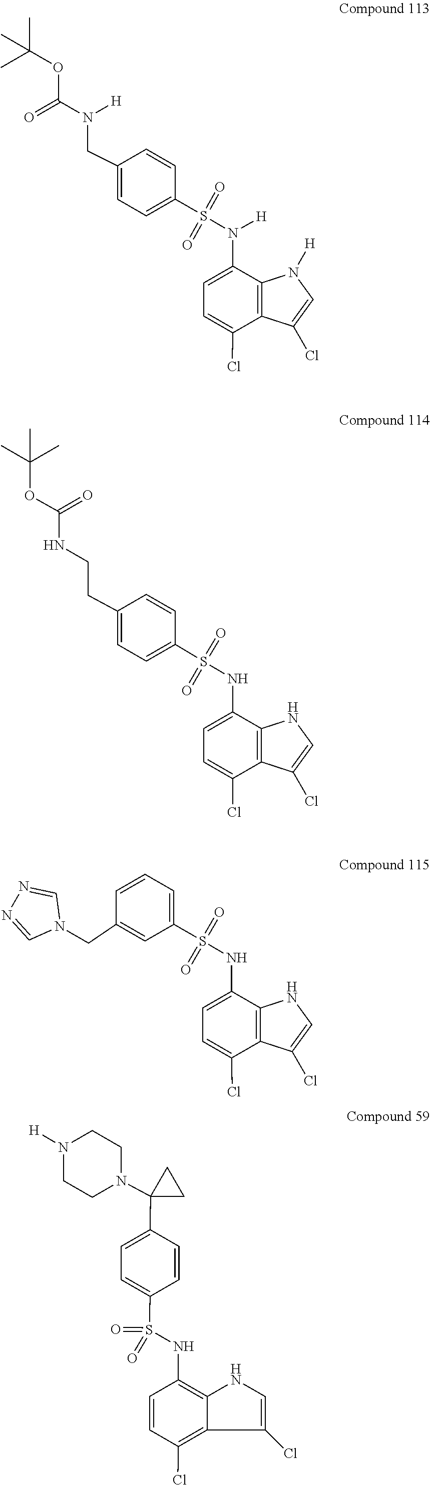 [SMILES: CC(C)(C)OC(=O)NCc1ccc(S(=O)(=O)Nc2ccc(Cl)c3c(Cl)c[nH]c23)cc1 ; CC(C)(C)OC(=O)NCCc1ccc(S(=O)(=O)Nc2ccc(Cl)c3c(Cl)c[nH]c23)cc1 ; O=S(=O)(Nc1ccc(Cl)c2c(Cl)c[nH]c12)c1cccc(Cn2cnnc2)c1 ; O=S(=O)(Nc1ccc(Cl)c2c(Cl)c[nH]c12)c1ccc(C2(N3CCNCC3)CC2)cc1]

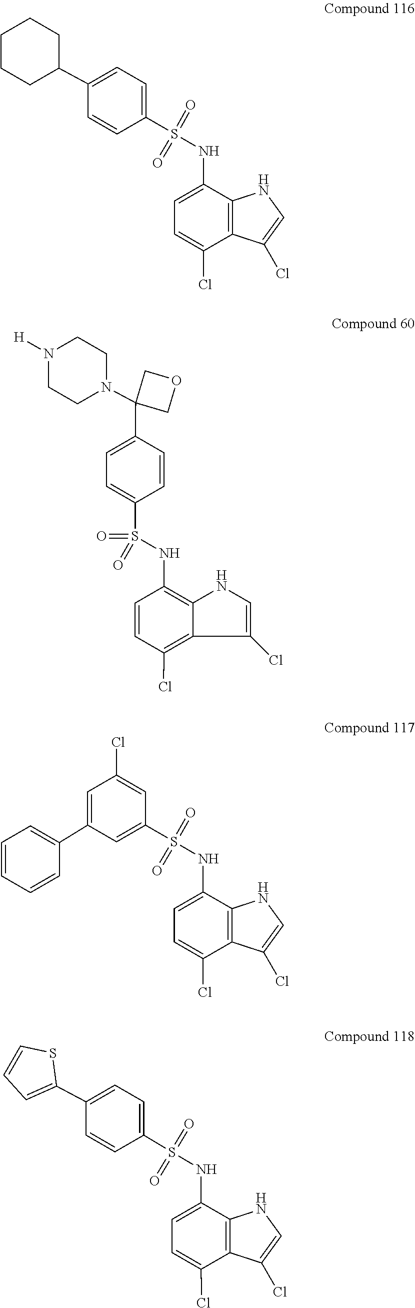 [SMILES: O=S(=O)(Nc1ccc(Cl)c2c(Cl)c[nH]c12)c1ccc(C2CCCCC2)cc1 ; O=S(=O)(Nc1ccc(Cl)c2c(Cl)c[nH]c12)c1ccc(C2(N3CCNCC3)COC2)cc1 ; O=S(=O)(Nc1ccc(Cl)c2c(Cl)c[nH]c12)c1cc(Cl)cc(-c2ccccc2)c1 ; O=S(=O)(Nc1ccc(Cl)c2c(Cl)c[nH]c12)c1ccc(-c2cccs2)cc1]

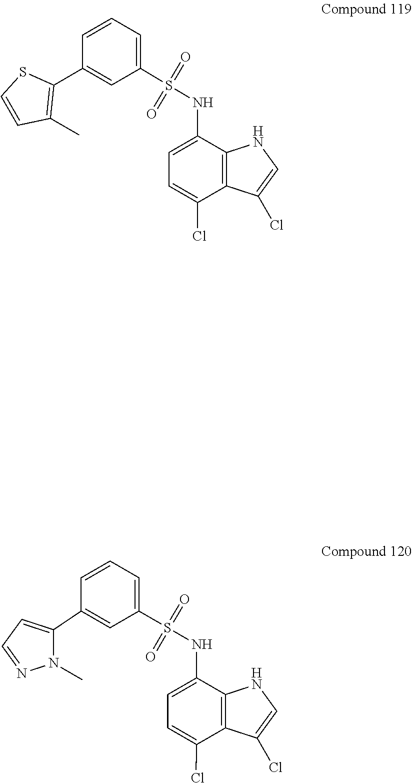 [SMILES: Cc1ccsc1-c1cccc(S(=O)(=O)Nc2ccc(Cl)c3c(Cl)c[nH]c23)c1 ; Cn1nccc1-c1cccc(S(=O)(=O)Nc2ccc(Cl)c3c(Cl)c[nH]c23)c1]

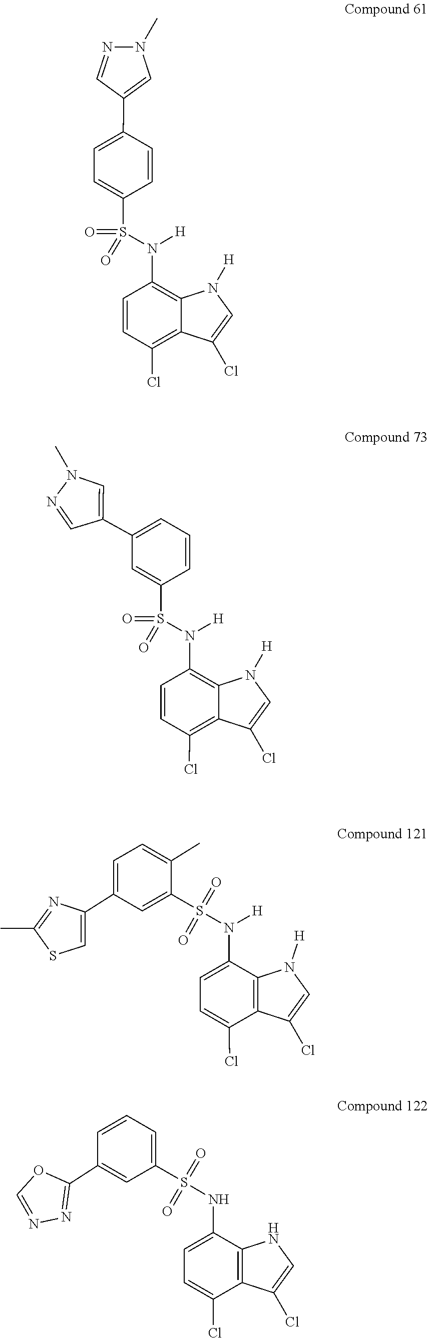 [SMILES: Cn1cc(-c2ccc(S(=O)(=O)Nc3ccc(Cl)c4c(Cl)c[nH]c34)cc2)cn1 ; Cn1cc(-c2cccc(S(=O)(=O)Nc3ccc(Cl)c4c(Cl)c[nH]c34)c2)cn1 ; Cc1nc(-c2ccc(C)c(S(=O)(=O)Nc3ccc(Cl)c4c(Cl)c[nH]c34)c2)cs1 ; O=S(=O)(Nc1ccc(Cl)c2c(Cl)c[nH]c12)c1cccc(-c2nnco2)c1]

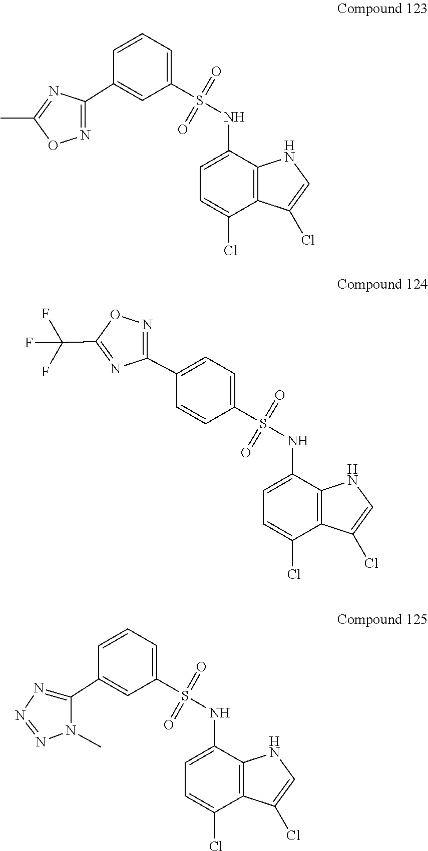 [SMILES: Cc1nc(-c2cccc(S(=O)(=O)Nc3ccc(Cl)c4c(Cl)c[nH]c34)c2)no1 ; O=S(=O)(Nc1ccc(Cl)c2c(Cl)c[nH]c12)c1ccc(-c2noc(C(F)(F)F)n2)cc1 ; Cn1nnnc1-c1cccc(S(=O)(=O)Nc2ccc(Cl)c3c(Cl)c[nH]c23)c1]

or a pharmaceutically acceptable salt thereof.

In some embodiments the compound of Formula I is a compound of Formula Vc, Formula Vd or Formula Ve

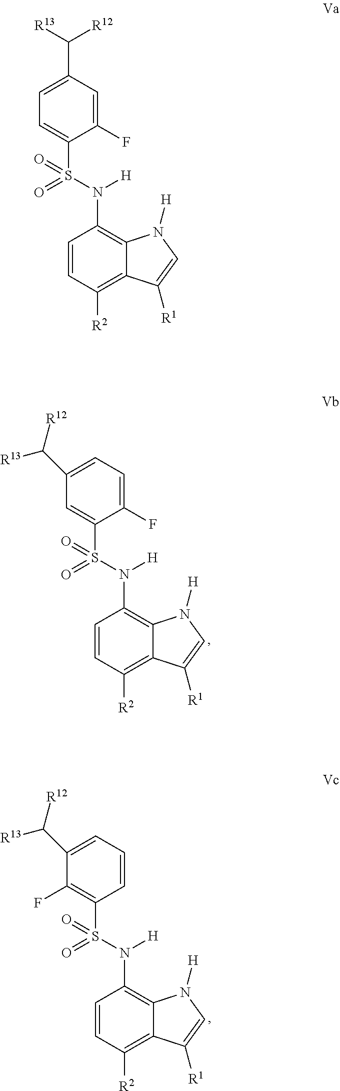

or a pharmaceutically acceptable salt thereof, wherein R¹, R², R¹², and R¹³, are as described according to any embodiment of compounds of Formula V.

### Compounds of Formula VI

In some embodiments the compound of Formula I is a compound of Formula VI

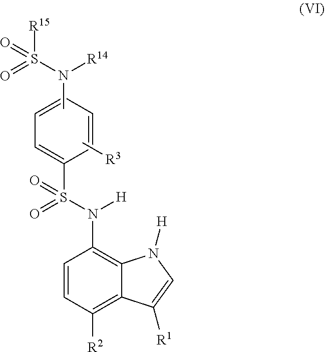

or a pharmaceutically acceptable salt thereof.

Substituent R¹ of Formula VI is selected from the group consisting of chloro, bromo, fluoro, and iodo. In some embodiments, R¹ is selected from the group consisting of chloro, bromo, and iodo. In some embodiments, R¹ is chloro.

Substituent R² of Formula VI is selected from the group consisting of H, chloro, fluoro and methyl. In some embodiments R² of Formula II is selected from the group consisting of H and chloro.

Substituent R³ of Formula VI is selected from the group consisting of H, chloro, fluoro, cyano and methyl. In some embodiments R³ is selected from the group consisting H and F. In some embodiments, R³ is H. In some embodiments, R³ is F.

Substituent R¹⁴ of Formula VI is selected from the group consisting of H and C₁-C₃ alkyl. In some embodiments, R¹⁴ is selected from the group consisting of H and propyl.

Substituent R¹⁵ of Formula VI is an optionally substituted 6 membered heterocyclyl. In some embodiments R¹⁵ is selected from the group consisting of piperidinyl and piperazinyl. In some embodiments R¹⁵ is selected from the group consisting of

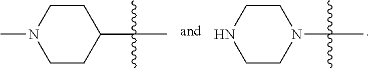

Some embodiments describe a compound of Formula VI wherein
R¹ is chloro;
R² is selected from the group consisting of H and chloro;
R³ is selected from the group consisting H and F;
R¹⁴ is selected from the group consisting of H and propyl; and
R¹⁵ is selected from the group consisting of

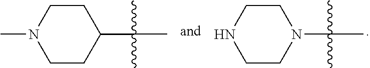

In some embodiments, the compound of Formula VI is a compound of Formula VIa or Formula VIb:

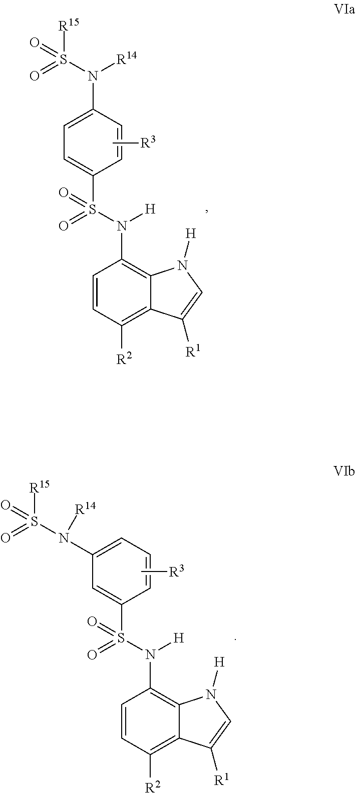

In some embodiments a compound of Formula VI is selected from

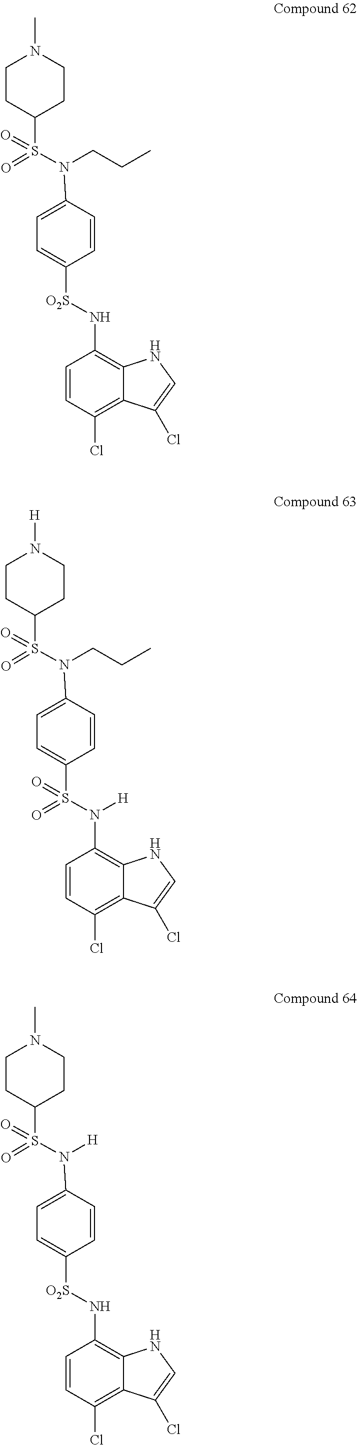 [SMILES: CCCN(c1ccc(S(=O)(=O)Nc2ccc(Cl)c3c(Cl)c[nH]c23)cc1)S(=O)(=O)C1CCN(C)CC1 ; CCCN(c1ccc(S(=O)(=O)Nc2ccc(Cl)c3c(Cl)c[nH]c23)cc1)S(=O)(=O)C1CCNCC1 ; CN1CCC(S(=O)(=O)Nc2ccc(S(=O)(=O)Nc3ccc(Cl)c4c(Cl)c[nH]c34)cc2)CC1]

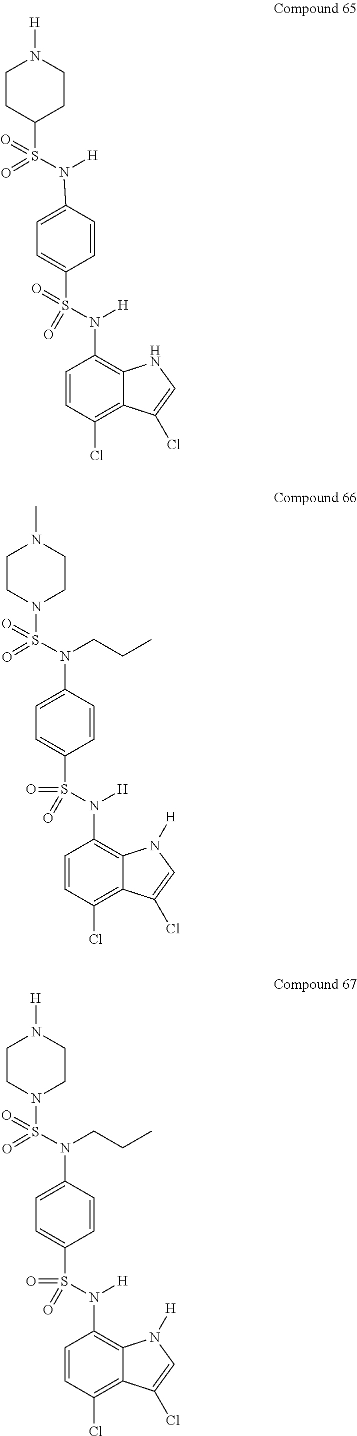 [SMILES: O=S(=O)(Nc1ccc(Cl)c2c(Cl)c[nH]c12)c1ccc(NS(=O)(=O)C2CCNCC2)cc1 ; CCCN(c1ccc(S(=O)(=O)Nc2ccc(Cl)c3c(Cl)c[nH]c23)cc1)S(=O)(=O)N1CCN(C)CC1 ; CCCN(c1ccc(S(=O)(=O)Nc2ccc(Cl)c3c(Cl)c[nH]c23)cc1)S(=O)(=O)N1CCNCC1]

or a pharmaceutically acceptable salt thereof.

In some embodiments the compound of Formula I is a compound of Formula VIc, Formula VId or Formula VIe:

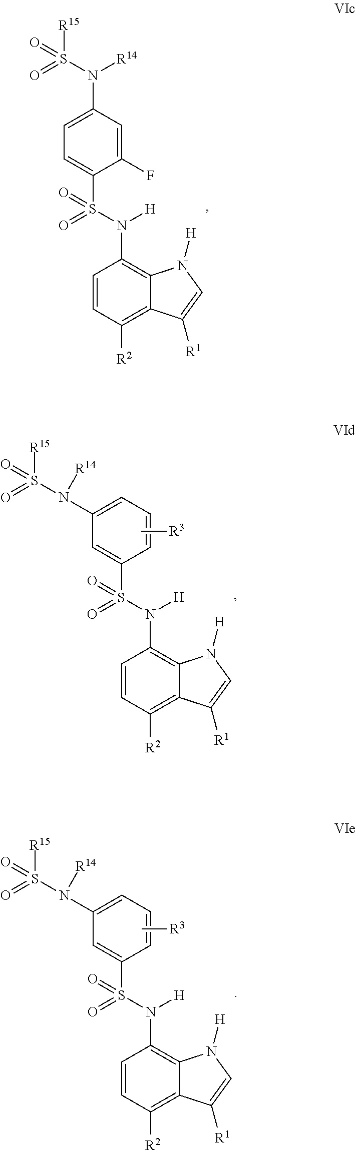

or a pharmaceutically acceptable salt thereof, wherein R¹, R², R¹⁴, and R¹⁵, are as described according to any embodiment of compounds of Formula VI.

### Compounds of Formula VII

In some embodiments the compound of Formula I is a compound of Formula VII

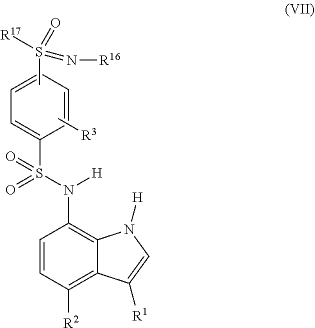

or a pharmaceutically acceptable salt thereof.

Substituent R¹ of Formula VII is selected from the group consisting of chloro, bromo, fluoro, and iodo. In some embodiments, R¹ is selected from the group consisting of chloro, bromo, and iodo. In some embodiments, R¹ is chloro.

Substituent R² of Formula VII is selected from the group consisting of H, chloro, fluoro and methyl. In some embodiments R² of Formula II is selected from the group consisting of H and chloro.

Substituent R³ of Formula VII is selected from the group consisting of H, chloro, fluoro, cyano and methy. In some embodiments R³ is selected from the group consisting H and F. In some embodiments, R³ is H. In some embodiments, R³ is F.

Substituent R¹⁶ of Formula VII is selected from the group consisting of H and C₁-C₃ alkyl. In some embodiments R¹⁶ is C₁-C₃ alky. In some embodiments, R¹⁶ is ethyl.

Substituent R¹⁷ of Formula VII is selected from the group consisting of C₁-C₆ alky, C₆-C₁₀ aryl and optionally substituted 6 membered heterocyclyl. In some embodiments R¹⁷ is an optionally substituted 6 membered heterocyclyl, In some embodiments R¹⁷ is selected from the group consisting of piperidinyl and piperazinyl. In some embodiments R¹⁷ is selected from the group consisting of

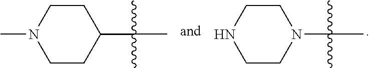

Some embodiments describe a compound of Formula VI wherein
R¹ is chloro;
R² is selected from the group consisting of H and chloro;
R³ is selected from the group consisting H and F;
R¹⁶ is ethyl; and
R¹⁷ is selected from the group consisting of

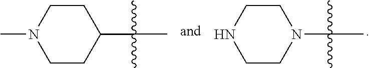

In some embodiments, the compound of Formula VII is a compound of Formula VIIa or Formula VIIb:

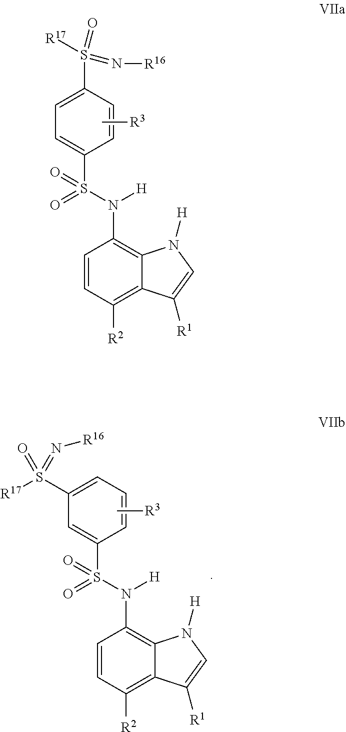

In some embodiments a compound of Formula VII is selected from

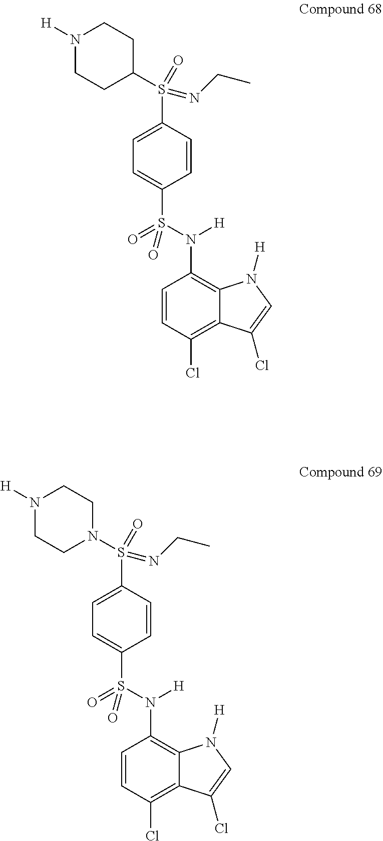 [SMILES: CCN=S(=O)(c1ccc(S(=O)(=O)Nc2ccc(Cl)c3c(Cl)c[nH]c23)cc1)C1CCNCC1 ; CCN=S(=O)(c1ccc(S(=O)(=O)Nc2ccc(Cl)c3c(Cl)c[nH]c23)cc1)N1CCNCC1]

or a pharmaceutically acceptable salt thereof.

In some embodiments the compound of Formula I is a compound of Formula VII-F

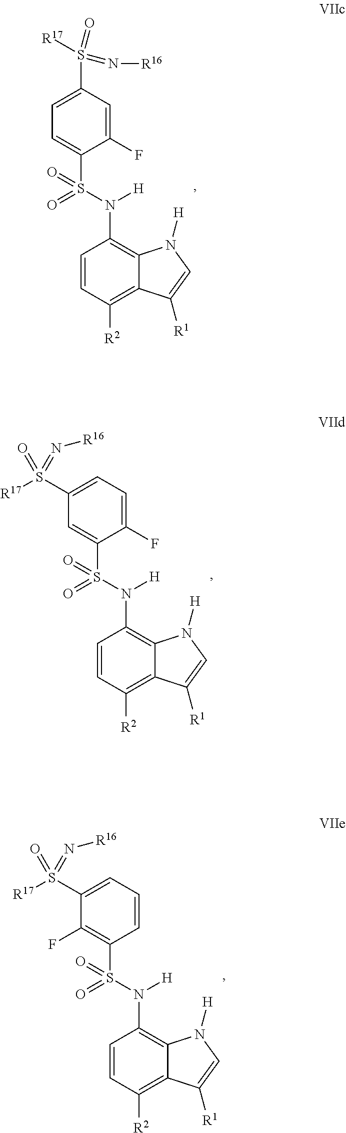

or a pharmaceutically acceptable salt thereof, wherein R¹, R², R¹⁶, and R¹⁷, are as described according to any embodiment of compounds of Formula VII.

### Compounds of Formula VIII

In some embodiments the compound of Formula I is a compound of Formula VIII

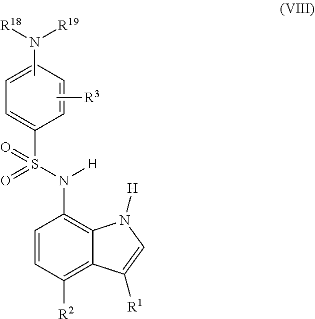

or a pharmaceutically acceptable salt thereof.

Substituent R¹ of Formula VIII is selected from the group consisting of chloro, bromo, fluoro, and iodo. In some embodiments, R¹ is selected from the group consisting of chloro, bromo, and iodo. In some embodiments, R¹ is chloro.

Substituent R² of Formula VIII is selected from the group consisting of H, chloro, fluoro and methyl. In some embodiments R² of Formula II is selected from the group consisting of H and chloro.

Substituent R³ of Formula VIII is selected from the group consisting of H, chloro, fluoro, cyano and methyl. In some embodiments R³ is selected from the group consisting H and F. In some embodiments, R³ is H. In some embodiments, R³ is F.

Substituent R¹⁸ and R¹⁹ together with the nitrogen to which they are attached form an optionally substituted 5 to 6-membered heteroaryl group. In some embodiments R¹⁸ and R¹⁹ together with the nitrogen to which they are attached form a group selected from optionally substituted tetrazolyl and optionally substituted pyridazinonyl. In some embodiments R¹⁸ and R¹⁹ together with the nitrogen to which they are attached form a group selected from

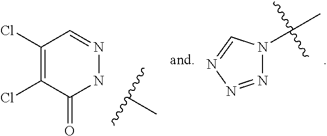

Some embodiments describe a compound of Formula VIII wherein
R¹ is chloro;
R² is selected from the group consisting of H and chloro;
R³ is selected from the group consisting H and F; and
R¹⁸ and R¹⁹ together with the nitrogen to which they are attached form a group selected from

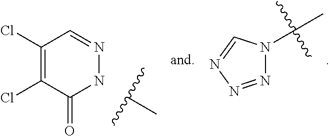

In some embodiments, the compound of Formula VIII is a compound of Formula VIIla or Formula VIIIb:

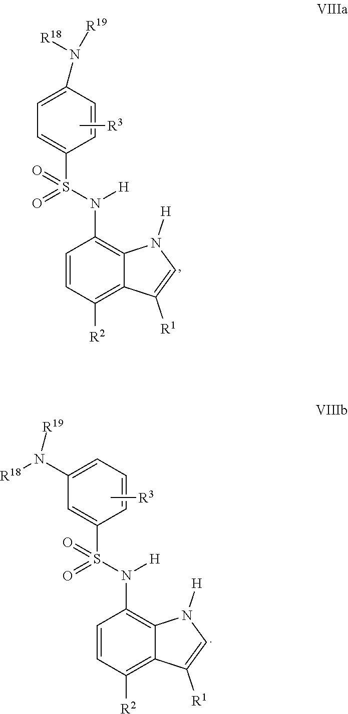

Some embodiments describe a compound of Formula VIII selected from

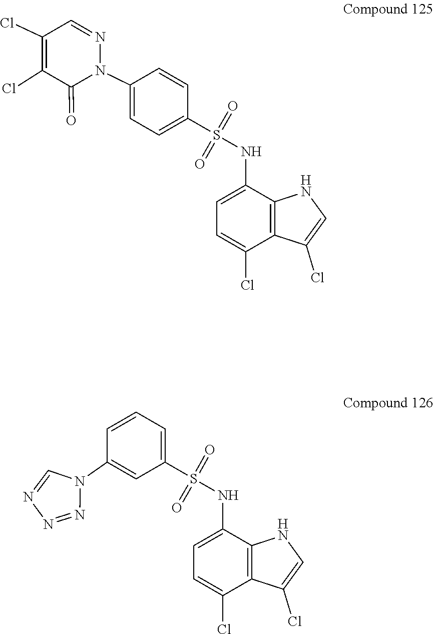 [SMILES: O=c1c(Cl)c(Cl)cnn1-c1ccc(S(=O)(=O)Nc2ccc(Cl)c3c(Cl)c[nH]c23)cc1 ; O=S(=O)(Nc1ccc(Cl)c2c(Cl)c[nH]c12)c1cccc(-n2cnnn2)c1]

or a pharmaceutically acceptable salt thereof.

In some embodiments the compound of Formula VIII is a compound of Formula VIIIc, Formula VIIId or Formula VIIIe:

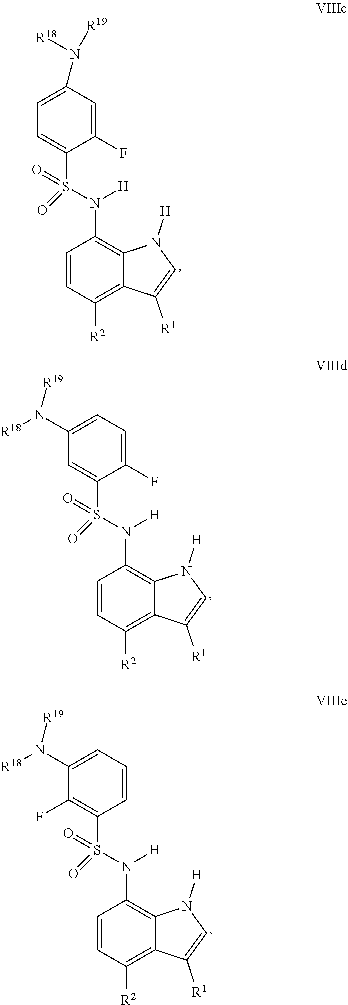

or a pharmaceutically acceptable salt thereof, wherein R¹, R², and R¹⁸, are as described according to any embodiment of compounds of Formula VIII.

### Additional Compounds

Some embodiments describe a compound selected from

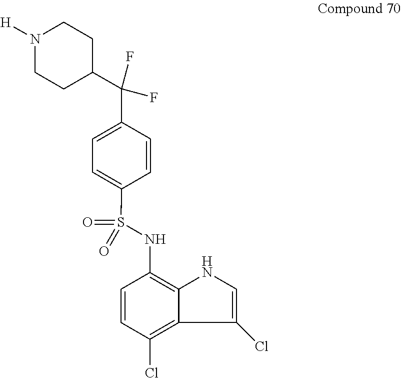 [SMILES: O=S(=O)(Nc1ccc(Cl)c2c(Cl)c[nH]c12)c1ccc(C(F)(F)C2CCNCC2)cc1]

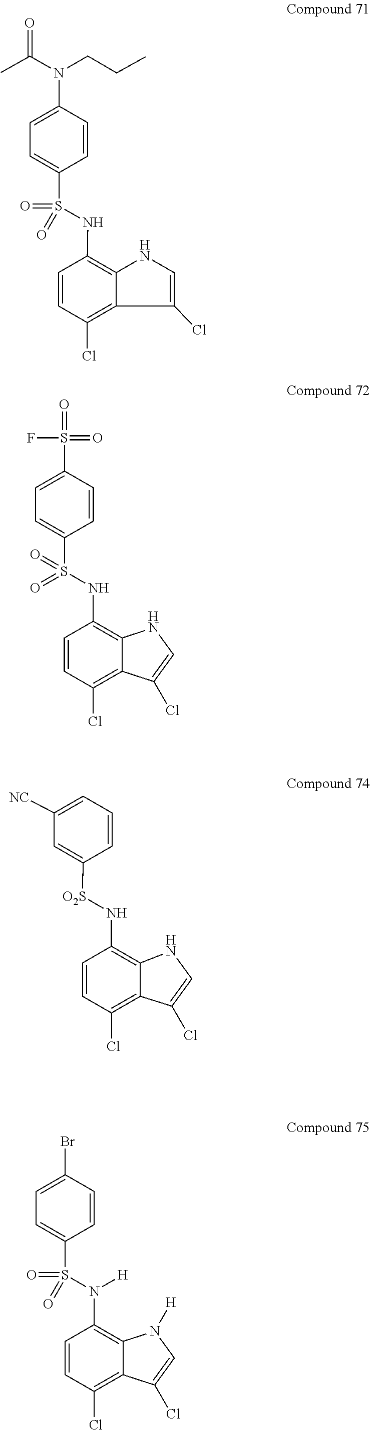 [SMILES: CCCN(C(C)=O)c1ccc(S(=O)(=O)Nc2ccc(Cl)c3c(Cl)c[nH]c23)cc1 ; O=S(=O)(F)c1ccc(S(=O)(=O)Nc2ccc(Cl)c3c(Cl)c[nH]c23)cc1 ; N#Cc1cccc(S(=O)(=O)Nc2ccc(Cl)c3c(Cl)c[nH]c23)c1 ; O=S(=O)(Nc1ccc(Cl)c2c(Cl)c[nH]c12)c1ccc(Br)cc1]

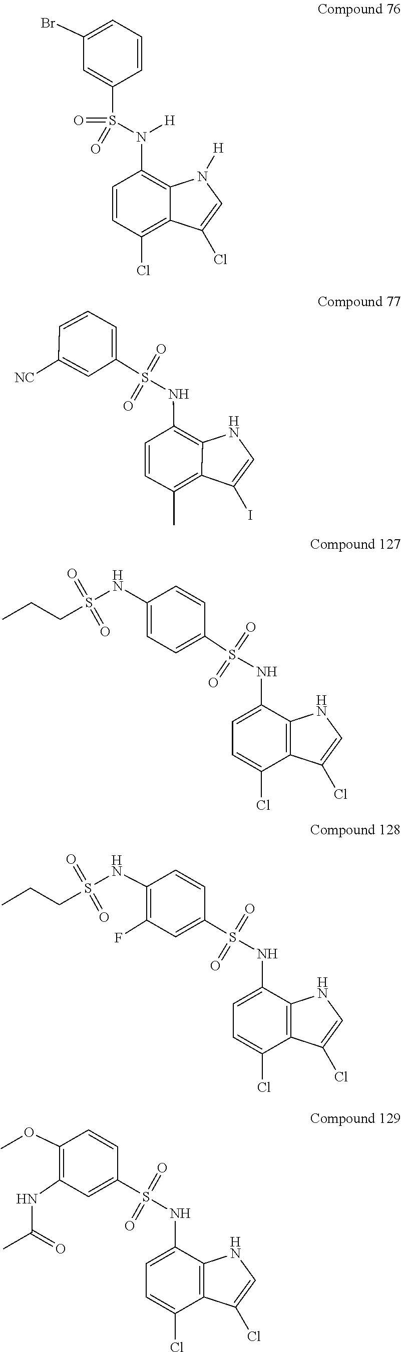 [SMILES: O=S(=O)(Nc1ccc(Cl)c2c(Cl)c[nH]c12)c1cccc(Br)c1 ; Cc1ccc(NS(=O)(=O)c2cccc(C#N)c2)c2[nH]cc(I)c12 ; CCCS(=O)(=O)Nc1ccc(S(=O)(=O)Nc2ccc(Cl)c3c(Cl)c[nH]c23)cc1 ; CCCS(=O)(=O)Nc1ccc(S(=O)(=O)Nc2ccc(Cl)c3c(Cl)c[nH]c23)cc1F ; COc1ccc(S(=O)(=O)Nc2ccc(Cl)c3c(Cl)c[nH]c23)cc1NC(C)=O]

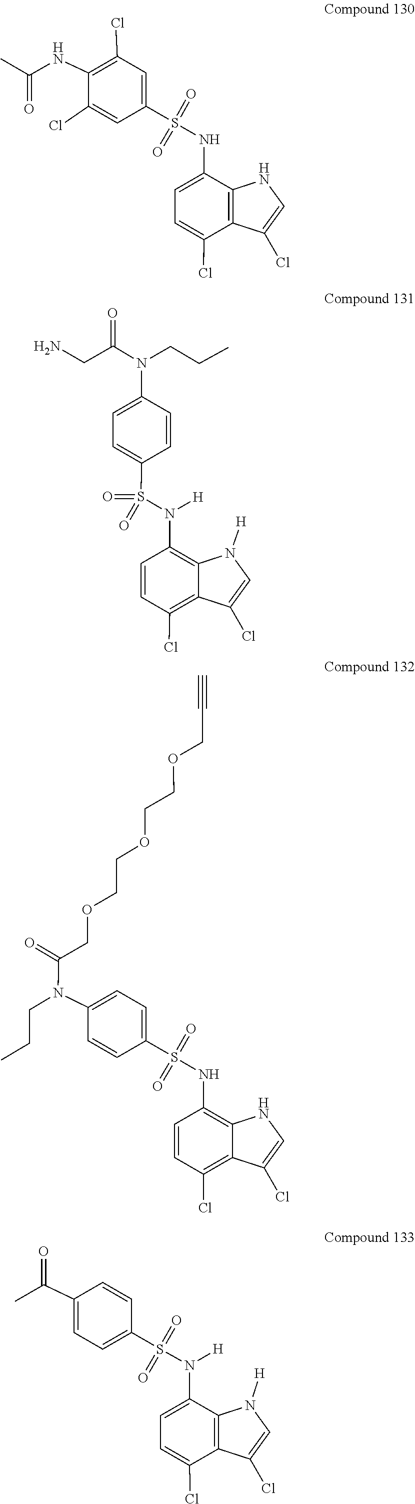

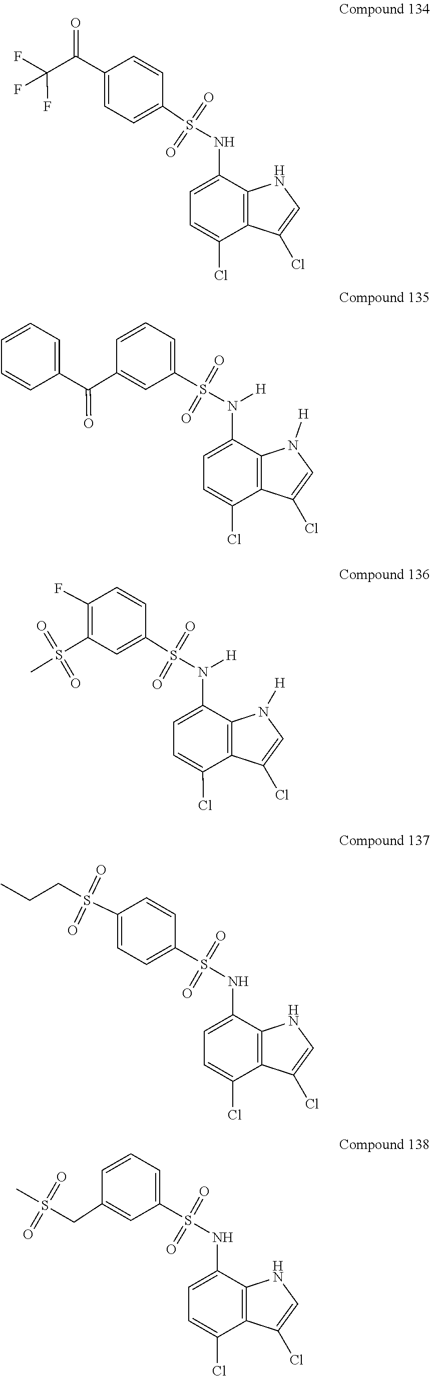 [SMILES: O=C(c1ccc(S(=O)(=O)Nc2ccc(Cl)c3c(Cl)c[nH]c23)cc1)C(F)(F)F ; O=C(c1ccccc1)c1cccc(S(=O)(=O)Nc2ccc(Cl)c3c(Cl)c[nH]c23)c1 ; CS(=O)(=O)c1cc(S(=O)(=O)Nc2ccc(Cl)c3c(Cl)c[nH]c23)ccc1F ; CCCS(=O)(=O)c1ccc(S(=O)(=O)Nc2ccc(Cl)c3c(Cl)c[nH]c23)cc1 ; CS(=O)(=O)Cc1cccc(S(=O)(=O)Nc2ccc(Cl)c3c(Cl)c[nH]c23)c1]

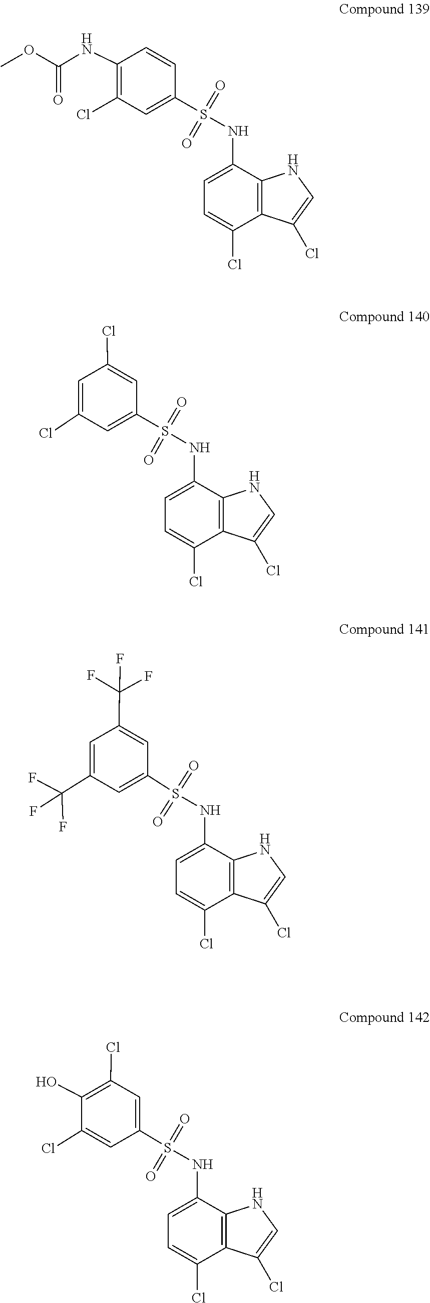 [SMILES: COC(=O)Nc1ccc(S(=O)(=O)Nc2ccc(Cl)c3c(Cl)c[nH]c23)cc1Cl ; O=S(=O)(Nc1ccc(Cl)c2c(Cl)c[nH]c12)c1cc(Cl)cc(Cl)c1 ; O=S(=O)(Nc1ccc(Cl)c2c(Cl)c[nH]c12)c1cc(C(F)(F)F)cc(C(F)(F)F)c1 ; O=S(=O)(Nc1ccc(Cl)c2c(Cl)c[nH]c12)c1cc(Cl)c(O)c(Cl)c1]

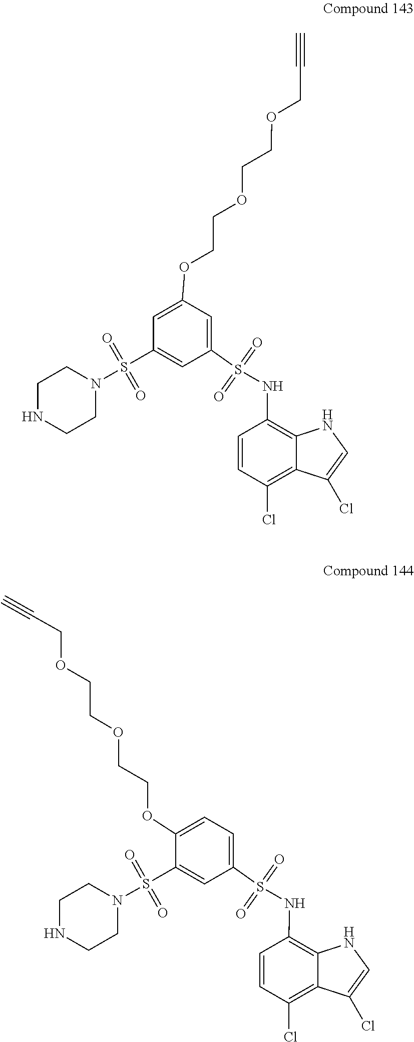 [SMILES: C#CCOCCOCCOc1cc(S(=O)(=O)Nc2ccc(Cl)c3c(Cl)c[nH]c23)cc(S(=O)(=O)N2CCNCC2)c1 ; C#CCOCCOCCOc1ccc(S(=O)(=O)Nc2ccc(Cl)c3c(Cl)c[nH]c23)cc1S(=O)(=O)N1CCNCC1]

Some embodiments describe a compound selected from

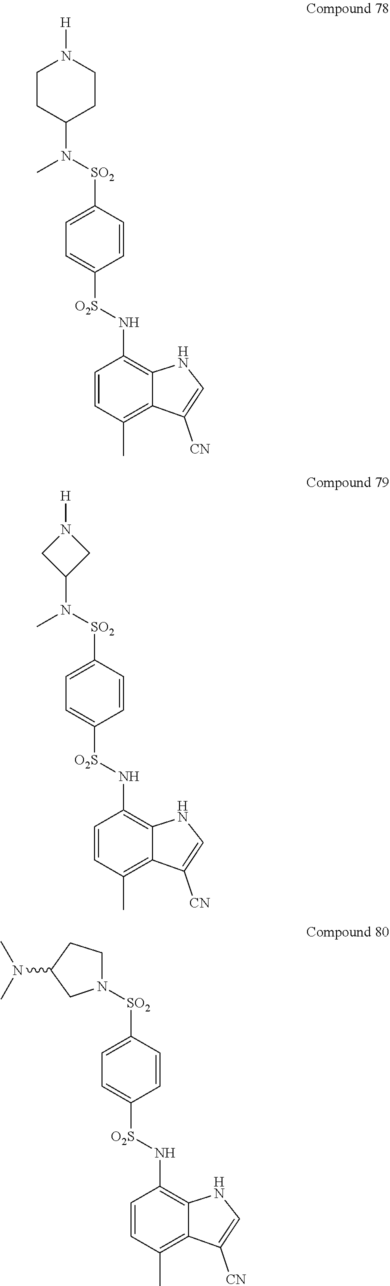 [SMILES: Cc1ccc(NS(=O)(=O)c2ccc(S(=O)(=O)N(C)C3CCNCC3)cc2)c2[nH]cc(C#N)c12 ; Cc1ccc(NS(=O)(=O)c2ccc(S(=O)(=O)N(C)C3CNC3)cc2)c2[nH]cc(C#N)c12 ; Cc1ccc(NS(=O)(=O)c2ccc(S(=O)(=O)N3CCC(N(C)C)C3)cc2)c2[nH]cc(C#N)c12]

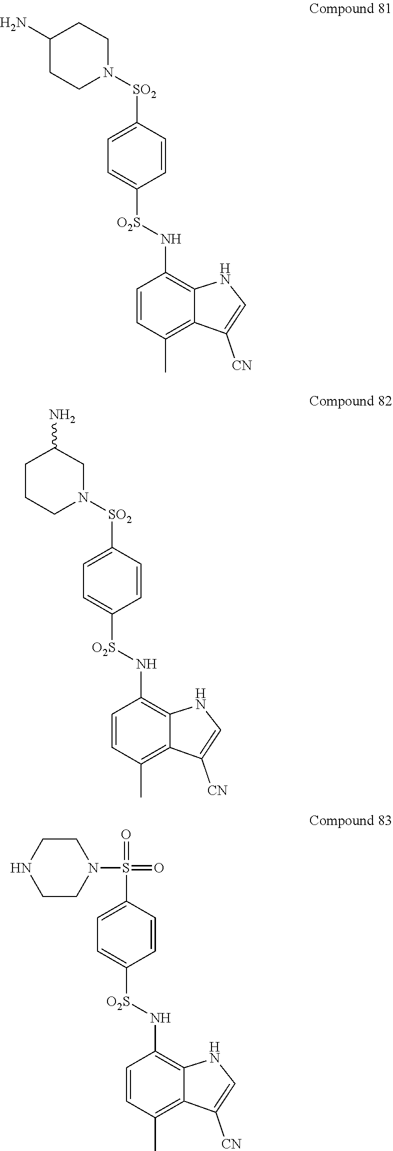

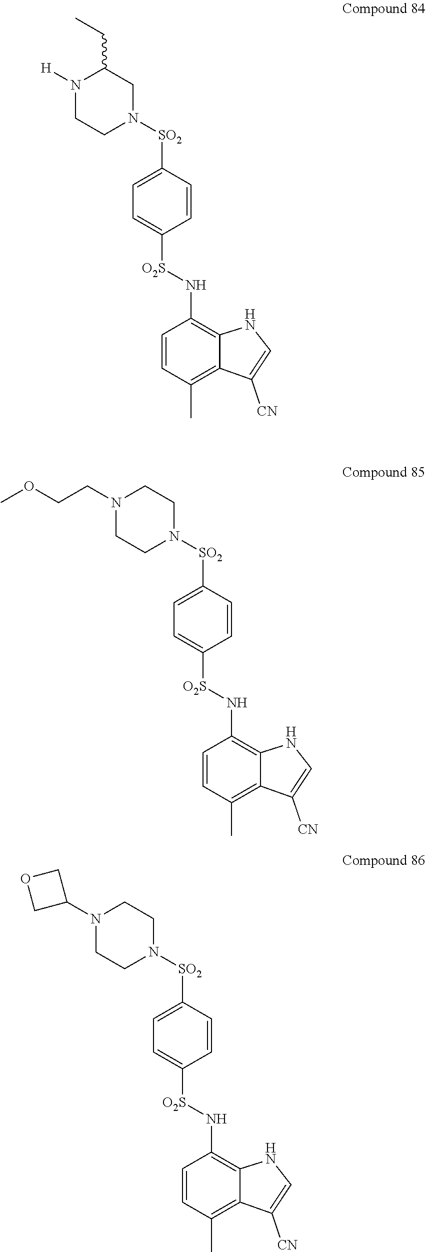 [SMILES: CCC1CN(S(=O)(=O)c2ccc(S(=O)(=O)Nc3ccc(C)c4c(C#N)c[nH]c34)cc2)CCN1 ; COCCN1CCN(S(=O)(=O)c2ccc(S(=O)(=O)Nc3ccc(C)c4c(C#N)c[nH]c34)cc2)CC1 ; Cc1ccc(NS(=O)(=O)c2ccc(S(=O)(=O)N3CCN(C4COC4)CC3)cc2)c2[nH]cc(C#N)c12]

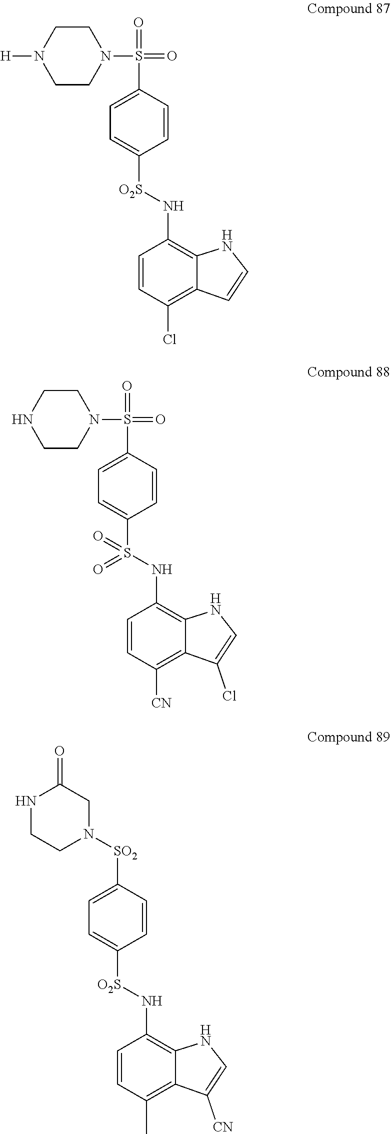 [SMILES: O=S(=O)(Nc1ccc(Cl)c2cc[nH]c12)c1ccc(S(=O)(=O)N2CCNCC2)cc1 ; N#Cc1ccc(NS(=O)(=O)c2ccc(S(=O)(=O)N3CCNCC3)cc2)c2[nH]cc(Cl)c12 ; Cc1ccc(NS(=O)(=O)c2ccc(S(=O)(=O)N3CCNC(=O)C3)cc2)c2[nH]cc(C#N)c12]

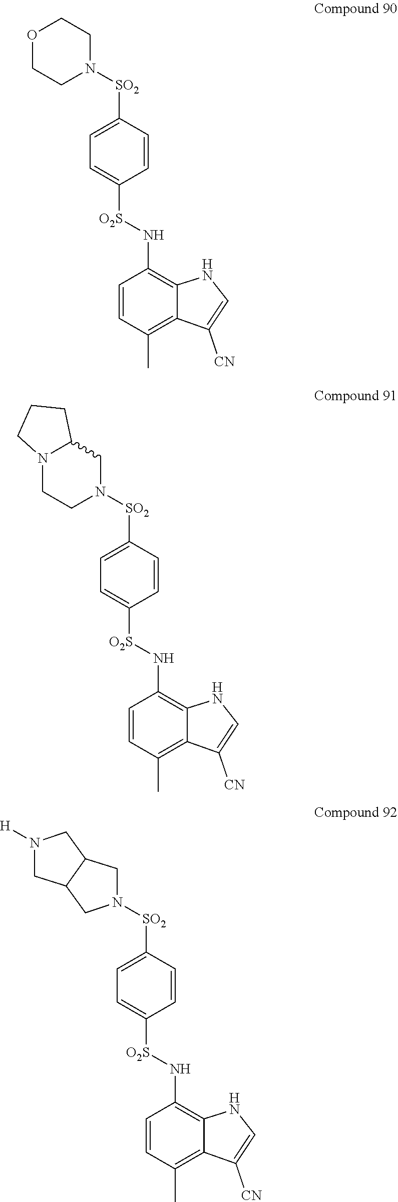 [SMILES: Cc1ccc(NS(=O)(=O)c2ccc(S(=O)(=O)N3CCOCC3)cc2)c2[nH]cc(C#N)c12 ; Cc1ccc(NS(=O)(=O)c2ccc(S(=O)(=O)N3CCN4CCCC4C3)cc2)c2[nH]cc(C#N)c12 ; Cc1ccc(NS(=O)(=O)c2ccc(S(=O)(=O)N3CC4CNCC4C3)cc2)c2[nH]cc(C#N)c12]

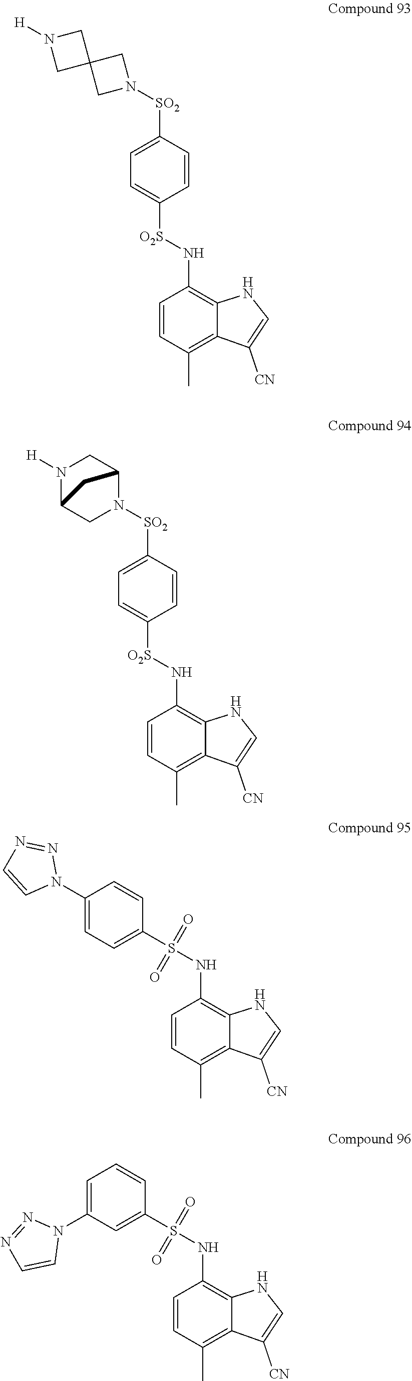

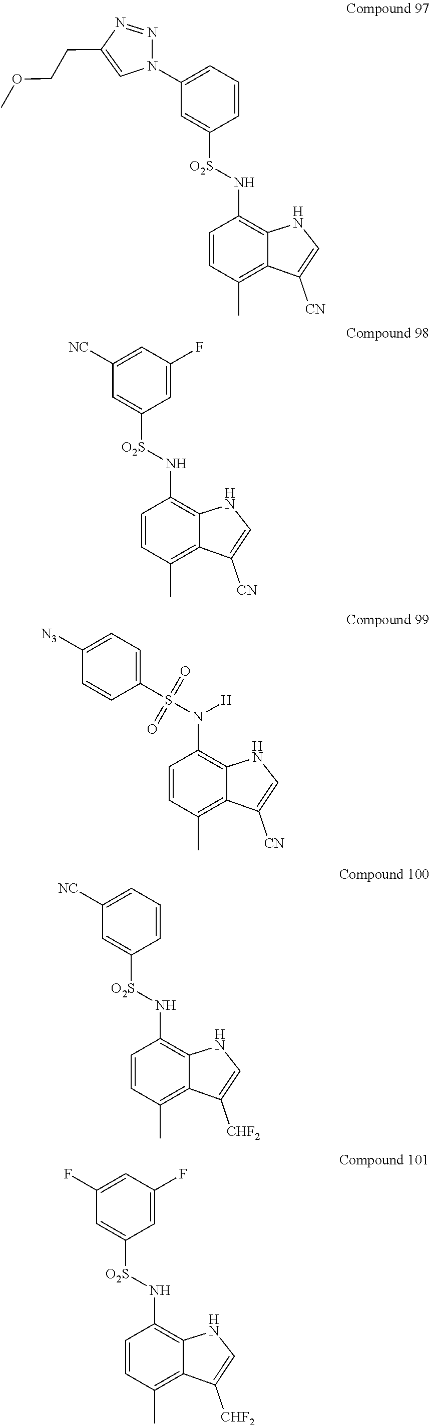

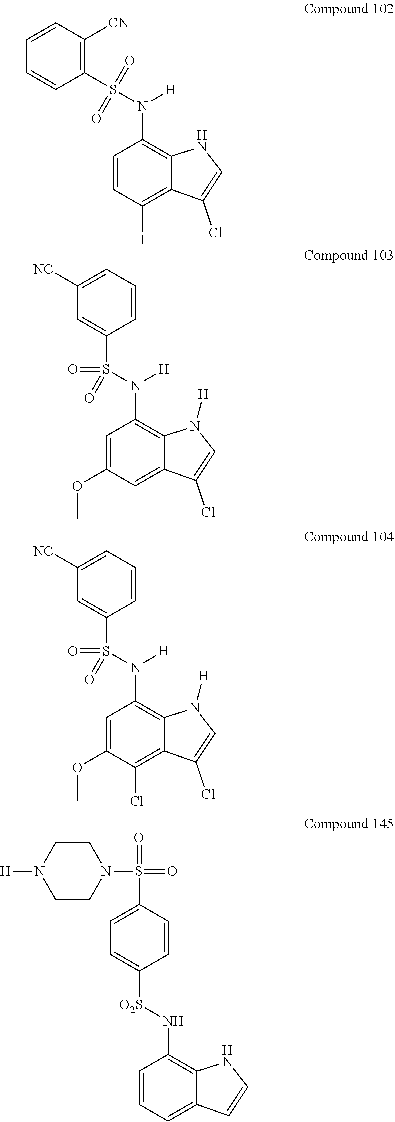

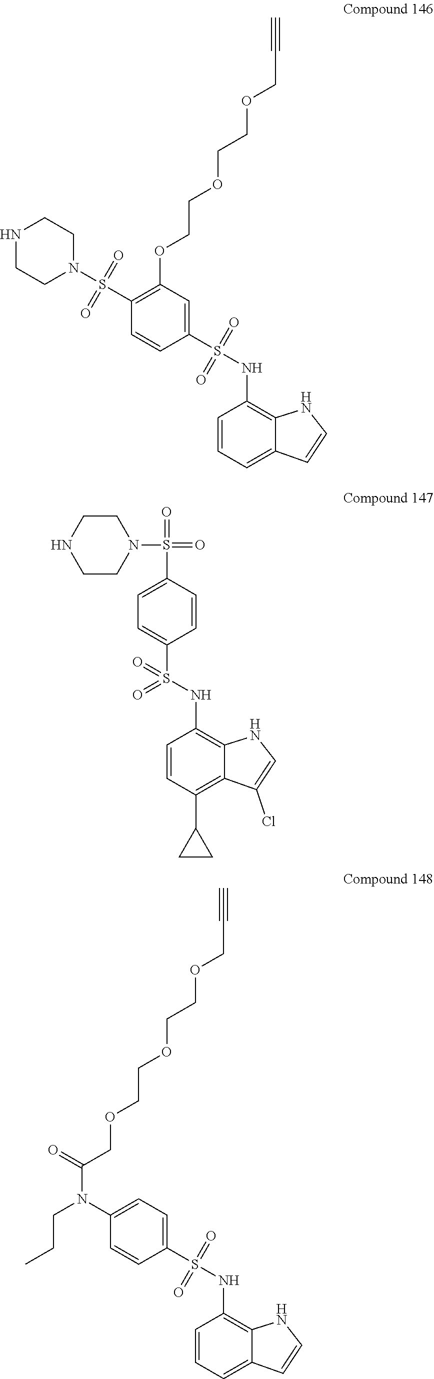

Some compounds of embodiments described herein can possess one or more asymmetric carbon atoms and are thus capable of existing in the form of optical isomers as well as in the form of racemic or non-racemic mixtures thereof. The compounds can be utilized in embodiments described herein as a single isomer or as a mixture of stereochemical isomeric forms. Diastereoisomers, i.e., non-superimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric and camphorsulfonic acid. The mixture of diastereomers can be separated by crystallization followed by liberation of the optically active bases from these salts. An alternative process for separation of optical isomers includes the use of a chiral chromatography column optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of the invention can likewise be obtained by utilizing optically active starting materials. These isomers may be in the form of a free acid, a free base, an ester or a salt.

Compounds according to embodiments described herein may be in the form of pharmaceutically acceptable salts. A pharmaceutically acceptable salt of the compounds described herein includes acid addition salts and base addition salts. Pharmaceutically-acceptable salt embraces salts commonly used to form alkali metal salts and to form addition salts of free acids or free bases The nature of the salt is not critical, provided that it is pharmaceutically-acceptable. Suitable pharmaceutically-acceptable acid addition salts of the compounds described herein may be prepared from an inorganic acid or an organic acid. Examples of such inorganic acids include, without limitation, hydrochloric, hydrobromic, hydroiodic, nitric, carbonic, sulfuric and phosphoric acid. In some embodiments the salt is a hydrochloride salt. Appropriate organic acids may be selected from aliphatic, cycloaliphatic, aromatic, arylaliphatic, heterocyclic, carboxylic and sulfonic classes of organic acids, examples of which include, without limitation, formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically-acceptable base addition salts for compounds described herein can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cyclo alkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful in the preparation of pharmaceutically acceptable salts, for example, carboxylic acid amides, including carboxamides, lower alkyl carboxamides, dialkyl carboxamides, and the like.

Acceptable salts may be obtained using standard procedures well known in the art, for example by treating a sufficiently basic compound such as an amine with a suitable acid affording a physiologically acceptable anion. Alkali metal (for example, sodium, potassium or lithium) or alkaline earth metal (for example calcium) salts of organic (e.g., carboxylic) acids can also be made.

In some aspects, compounds of embodiments herein may exist in both unsolvated and solvated forms. The term solvate is used herein to describe a molecular complex comprising a compound of embodiments herein and an amount of one or more pharmaceutically acceptable solvent molecules. The term hydrate is employed when said solvent is water.

Furthermore, it is specifically contemplated that in embodiments herein, more than one solvent molecule may be associated with one molecule of the compounds of embodiments herein, such as a dihydrate. Additionally, it is specifically contemplated that in embodiments herein less than one solvent molecule may be associated with one molecule of the compounds of embodiments herein, such as a hemihydrate. Furthermore, solvates of embodiments herein are contemplated as solvates of compounds of embodiments herein that retain the biological effectiveness of the non-solvate form of the compounds.

In one aspect, embodiments herein also include isotopically-labeled compounds of embodiments herein, wherein one or more atoms is replaced by an atom having the same atomic number, but an atomic mass or mass number different from the atomic mass or mass number usually found in nature. Examples of isotopes suitable for inclusion in the compounds of embodiments herein include isotopes of hydrogen, such as ²H and ³H, carbon, such as ¹¹C, ¹³C and ¹⁴C, chlorine, such as ³¹Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, and sulfur, such as ³⁵S. Certain isotopically-labeled compounds of embodiments herein, for example, those incorporating a radioactive isotope, are useful in drug and/or substrate tissue distribution studies. The radioactive isotopes tritium, ³H, and carbon-14, ¹⁴C, are particularly useful for this purpose in view of their ease of incorporation and ready means of detection. Substitution with heavier isotopes such as deuterium, ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of embodiments herein can generally be prepared by conventional techniques known to those skilled in the art or by processes analogous to those described herein, using an appropriate isotopically-labeled reagent in place of the non-labeled reagent otherwise employed.

Preferred isotopically-labeled compounds include deuterated derivatives of the compounds of embodiments herein. As used herein, the term deuterated derivative embraces compounds of embodiments herein where in a particular position at least one hydrogen atom is replaced by deuterium. Deuterium (D or ²H) is a stable isotope of hydrogen which is present at a natural abundance of 0.015 molar %. It is well established that deuteration of physiologically active compounds offer the advantage of retaining the pharmacological profile of their hydrogen counterparts while positively impacting their metabolic outcome. Selective replacement of one or more hydrogen with deuterium, in a compound of the present invention, could improve the safety, tolerability and efficacy of the compound when compared to its all hydrogen counterpart.

Methods for incorporation of deuterium into compounds is well established. Using metabolic studies establish in the art, the compound of the present invention can be tested to identify sites for selective placement of a deuterium isotope, wherein the isotope will not be metabolized. Moreover these studies identify sites of metabolism as the location where a deuterium atom would be placed.

Hydrogen deuterium exchange (deuterium incorporation) is a chemical reaction in which a covalently bonded hydrogen atom is replaced by a deuterium atom. Said exchange (incorporation) reaction can be total or partial.

Typically, a deuterated derivative of a compound of embodiments herein has an isotopic enrichment factor (ratio between the isotopic abundance and the natural abundance of that isotope, i.e. the percentage of incorporation of deuterium at a given position in a molecule in the place of hydrogen) for each deuterium present at a site designated as a potential site of deuteration on the compound of at least 3500 (52.5% deuterium incorporation).

In some embodiments, the isotopic enrichment factor is at least 5000 (75% deuterium). In some embodiments, the isotopic enrichment factor is at least 6333.3 (95% deuterium incorporation). In some embodiments, the isotopic enrichment factor is at least 6633.3 (99.5% deuterium incorporation). It is understood that the isotopic enrichment factor of each deuterium present at a site designated as a site of deuteration is independent from the other deuteration sites.

The isotopic enrichment factor can be determined using conventional analytical methods known to one of ordinary skilled in the art, including mass spectrometry (MS) and nuclear magnetic resonance (NMR).

In certain aspects, prodrugs of the compounds described herein are also within the scope of embodiments herein. Thus, certain derivatives of the compounds of embodiments herein, which derivatives may have little or no pharmacological activity themselves, when administered into or onto the body may be converted into compounds of embodiments herein having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with embodiments herein can, for example, be produced by replacing appropriate functionalities present in the compounds of embodiments herein with certain moieties known to those skilled in the art as ‘pro-moieties’ as described, for example, in Design of Prodrugs by H. Bundgaard (Elsevier, 1985).

In the case of compounds of embodiments herein that are solids, it is understood by those skilled in the art that the inventive compounds and salts may exist in different crystalline or polymorphic forms, or in an amorphous form, all of which are intended to be within the scope of embodiments herein.

The compounds disclosed herein can exist as and therefore include all tautomers, and mixtures thereof in all proportions as well as isotopic forms such as deuterated compounds.

The invention also embraces isolated compounds. An isolated compound refers to a compound which represents at least 10%, preferably at least 20%, more preferably at least 50% and most preferably at least 80% of the compound present in the mixture.

### Pharmaceutical Compositions

Some embodiments describe a pharmaceutical composition comprising: a compound according to any embodiment described herein, a pharmaceutically acceptable salt thereof, a solvate thereof, a stereoisomer thereof, a prodrug thereof, or an active metabolites thereof; and a pharmaceutically acceptable carrier or diluent. The pharmaceutical compositions can be prepared in a manner well known in the pharmaceutical art, and can be administered by a variety of routes, depending upon whether local or systemic treatment is desired and upon the area to be treated.

While it is possible that a compound as described in any embodiment herein, may be administered as the bulk substance, it is preferable to present the compound in a pharmaceutical formulation, e.g., wherein the active agent is in an admixture with a pharmaceutically acceptable carrier selected with regard to the intended route of administration and standard pharmaceutical practice.

In particular, the disclosure provides a pharmaceutical composition comprising a therapeutically effective amount of at least one compound according to any embodiment described herein, and optionally, a pharmaceutically acceptable carrier.

### Combinations

For the pharmaceutical compositions and methods of the disclosure, a compound according to any embodiment described herein, may be used in combination with other therapies and/or active agents.

Accordingly, the disclosure provides, in a further aspect, a pharmaceutical composition comprising at least one compound according to any embodiment described herein, or pharmaceutically acceptable derivative thereof; a second active agent; and, optionally a pharmaceutically acceptable carrier.

When used in combination therapy, the compounds described herein are administered with the second active agent simultaneously or separately. Thus the combination of the two agents can be administered simultaneously in the same dosage form, simultaneously in separate dosage forms or administered separately. When combined in the same formulation it will be appreciated that the two or more compounds must be stable and compatible with each other and the other components of the formulation. When formulated separately they may be provided in any convenient formulation, in such a manner as are known for such compounds in the art. In some embodiments, a compound according to any embodiment described herein is administered first, followed by administration of the second agent. In some embodiments the second agent is administered first, followed by administration of the second. In some embodiments the administration of the two agents can begin simultaneously, but is not completed at the same time. For example, one agent is administered orally, simultaneously with commencement of the administration of a second agent via a thirty minute bolus IV. In some embodiments a compound according to embodiments described herein and a second active agent are administered separately a few minutes apart, a few hours apart or a few days apart.

Preservatives, stabilizers, dyes and flavoring agents may be provided in any pharmaceutical composition described herein. Examples of preservatives include sodium benzoate, ascorbic acid and esters of p-hydroxybenzoic acid. Antioxidants and suspending agents may be also used.

### Routes of Administration and Unit Dosage Forms

Compounds according to any embodiment described herein, or pharmaceutically acceptable salts thereof, a solvate thereof, a stereoisomer thereof, a prodrug thereof, or an active metabolites thereof, can be formulated for any route of administration.

The routes for administration (delivery) include, but are not limited to, one or more of: oral (e.g., as a tablet, capsule, or as an ingestible solution), topical, mucosal (e.g., as a nasal spray or aerosol for inhalation), parenteral (e.g., by an injectable form), gastrointestinal, intraspinal, intraperitoneal, intramuscular, intravenous, intracerebroventricular, or other depot administration etc.

Therefore, the pharmaceutical compositions according to any embodiment described herein, include those in a form especially formulated for the mode of administration. In certain embodiments, the pharmaceutical compositions of the disclosure are formulated in a form that is suitable for oral delivery. In some embodiments, the compound is an orally bioavailable compound, suitable for oral delivery. In other embodiments, the pharmaceutical compositions of the disclosure are formulated in a form that is suitable for parenteral delivery.

The compounds according to any embodiment described herein, may be formulated for administration in any convenient way for use in human or veterinary medicine and the disclosure therefore includes within its scope pharmaceutical compositions comprising a compound according to any embodiment described herein, adapted for use in human or veterinary medicine. Such pharmaceutical compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

There may be different pharmaceutical composition/formulation requirements depending on the different delivery systems. It is to be understood that not all of the compounds need to be administered by the same route. Likewise, if the pharmaceutical composition comprises more than one active component, then those components may be administered by different routes. By way of example, the pharmaceutical composition of the disclosure may be formulated to be delivered using a mini-pump or by a mucosal route, for example, as a nasal spray or aerosol for inhalation or ingestible solution, or parenterally in which the pharmaceutical composition is formulated by an injectable form, for delivery by, for example, an intravenous, intramuscular or subcutaneous route. Alternatively, the formulation may be designed to be delivered by multiple routes.

Where appropriate, the pharmaceutical compositions according to any embodiment described herein, can be administered by inhalation, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For buccal or sublingual administration the pharmaceutical compositions according to any embodiment described herein, may be administered in the form of tablets or lozenges, which can be formulated in a conventional manner.

Where the pharmaceutical composition according to any embodiment described herein, is to be administered parenterally, such administration includes without limitation: intravenously, intraarterially, intrathecally, intraventricularly, intracranially, intramuscularly or subcutaneously administering the compound of the disclosure; and/or by using infusion techniques.

Pharmaceutical compositions according to any embodiment described herein, suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or ascorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminum monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the compound according to any embodiment described herein, is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing a compound according to any embodiment described herein, with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the compounds and desired excipients for subsequent preparation of sterile solutions.

The compounds according to any embodiment described herein, may be formulated for use in human or veterinary medicine by injection (e.g., by intravenous bolus injection or infusion or via intramuscular, subcutaneous or intrathecal routes) and may be presented in unit dose form, in ampoules, or other unit-dose containers, or in multi-dose containers, if necessary with an added preservative. The pharmaceutical compositions for injection may be in the form of suspensions, solutions, or emulsions, in oily or aqueous vehicles, and may contain formulatory agents such as suspending, stabilizing, solubilizing and/or dispersing agents. Alternatively the active ingredient may be in sterile powder form for reconstitution with a suitable vehicle, e.g., sterile, pyrogen-free water, before use.

The compounds according to any embodiment described herein, can be administered in the form of tablets, capsules, troches, ovules, elixirs, solutions or suspensions, for immediate-, delayed-, modified-, sustained-, pulsed- or controlled-release applications.

The compounds according to any embodiment described herein, may also be presented for human or veterinary use in a form suitable for oral or buccal administration, for example in the form of solutions, gels, syrups, or suspensions, or a dry powder for reconstitution with water or other suitable vehicle before use. Solid pharmaceutical compositions such as tablets, capsules, lozenges, troches, pastilles, pills, boluses, powder, pastes, granules, bullets or premix preparations may also be used. Solid and liquid pharmaceutical compositions for oral use may be prepared according to methods well-known in the art. Such pharmaceutical compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The pharmaceutical compositions according to any embodiment described herein, may be administered orally, in the form of rapid or controlled release tablets, microparticles, mini tablets, capsules, sachets, and oral solutions or suspensions, or powders for the preparation thereof. Oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the pharmaceutical compositions, e.g., act as both binders and disintegrants.

Examples of pharmaceutically acceptable disintegrants for oral pharmaceutical compositions according to any embodiment described herein, include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and cross-linked polyvinylpyrrolidone.

Examples of pharmaceutically acceptable binders for oral pharmaceutical compositions according to any embodiment described herein, include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthine resin, alginates, magnesium-aluminum silicate, polyethylene glycol or bentonite.

Examples of pharmaceutically acceptable fillers for oral pharmaceutical compositions according to any embodiment described herein, include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulphate.

Examples of pharmaceutically acceptable lubricants useful in the pharmaceutical compositions according to any embodiment described herein, include, but are not limited to, magnesium stearate, talc, polyethylene glycol, polymers of ethylene oxide, sodium lauryl sulphate, magnesium lauryl sulphate, sodium oleate, sodium stearyl fumarate, and colloidal silicon dioxide.

Examples of suitable pharmaceutically acceptable odorants for the oral pharmaceutical compositions according to any embodiment described herein, include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral pharmaceutical compositions according to any embodiment described herein, include, but are not limited to, synthetic and natural dyes such as titanium dioxide, beta-carotene and extracts of grapefruit peel.

Examples of useful pharmaceutically acceptable coatings for the oral pharmaceutical compositions according to any embodiment described herein, typically used to facilitate swallowing, modify the release properties, improve the appearance, and/or mask the taste of the pharmaceutical compositions include, but are not limited to, hydroxypropylmethylcellulose, hydroxypropylcellulose and acrylate-methacrylate copolymers.

Suitable examples of pharmaceutically acceptable sweeteners for the oral pharmaceutical compositions according to any embodiment described herein, include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulphate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

As indicated, a compounds according to any embodiment described herein, can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of a compound according to any embodiment described herein, and a suitable powder base such as lactose or starch.

For topical administration by inhalation a compounds according to any embodiment described herein, may be delivered for use in human or veterinary medicine via a nebulizer.

The pharmaceutical compositions of the disclosure may contain from 0.01 to 99% weight per volume of the active material. For topical administration, for example, the pharmaceutical composition will generally contain from 0.01-10%, more preferably 0.01-1% of the active material.

A compound according to any embodiment described herein, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The pharmaceutical composition or unit dosage form, according to any embodiment described herein, may be administered according to a dosage and administration regimen defined by routine testing in the light of the guidelines given above in order to obtain optimal activity while minimizing toxicity or side effects for a particular patient. The dosage of the compounds or unit dosage form may vary according to a variety of factors such as underlying disease conditions, the individual's condition, weight, sex and age, and the mode of administration. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter is within the scope of the disclosure. Clinically significant attenuation or amelioration means perceptible to the patient and/or to the physician.

A pharmaceutical composition for parenteral administration contains from about 0.01% to about 100% by weight of the active compound according to any embodiment described herein, based upon 100% weight of total pharmaceutical composition.

Generally, transdermal dosage forms contain from about 0.01% to about 100% by weight of the active compound according to any embodiment described herein, versus 100% total weight of the dosage form.

The pharmaceutical composition or unit dosage form may be administered in a single daily dose, or the total daily dosage may be administered in divided doses. In addition, co administration or sequential administration of another compound for the treatment of the disorder may be desirable. To this purpose, the combined active principles are formulated into a simple dosage unit.

### Methods of Use

In some embodiments, the present disclosure provides a method of inducing degradation of a protein comprising contacting the protein with an effective amount of a compound or composition according to any embodiment described herein.

Without wishing to be bound by any particular theory, the inventors believe that a compound according to any embodiment herein, can act as a molecular glue and induce an interaction between a ubiquitin E3 ligase and a target protein, leading to degradation of the target protein. Inducing protein degradation results in a decrease in protein levels. In some embodiments, the compounds promotes the formation of a complex between the protein and a substrate recognition subunit of the E3 ligase. In some instances a compound according to any embodiment herein binds to the ubiquitin E3 ligase and recruits proteins for degradation by the ubiquitin-proteasome system. In some embodiments the substrate recognition subunit of the E3 ligase is DCAF15. In some embodiments the substrate recognition subunit of the E3 ligase is human DCAF15. An example of human DCAF15 is Uniprot ID: Q66K64. In some embodiments the compound binds to the protein. In some embodiments the protein is a protein that may lack enzymatic active sites that can be targeted by inhibitors. In some embodiments the protein is RBM39. In some embodiments the substrate recognition subunit of the E3 ligase is DCAF15 and the protein is RBM39.

Some embodiments describe a method of inducing degradation of a protein, in a patient in need thereof, comprising administering to the patient, a therapeutically effective amount of a compound according to any embodiment described herein.

In some embodiments the compounds and compositions according to embodiments of the present invention are useful for treating a disease, disorder or condition associated with a target protein. Any disease, disorder or condition that results directly or indirectly from abnormal activity of the target protein or expression level of the gene that encodes the target protein can be an intended disease condition. Some embodiments describe a method of treating a disease or disorder that results from abnormal activity of a target protein in a subject, comprising administering to the subject, a therapeutically effective amount of a compound according to any embodiment described herein, wherein the compound induces degradation of the protein thereby treating the disease or disorder. In some embodiments the disease or disorder results directly from abnormal activity of a target protein. In some embodiments the disease or disorder results indirectly from the abnormal activity of the target protein. Some embodiments describe a method of treating a disease or disorder that results from abnormal expression of a gene that encodes a target protein in a subject, comprising administering to the subject, a therapeutically effective amount of a compound according to any embodiment described herein, wherein the compound induces degradation of the protein thereby treating the disease or disorder.

Some embodiments of the invention describe use of a compound according to any embodiment described herein, in the preparation of a medicament for promoting the degradation of a protein, in a patient in need thereof. Some embodiments describe a compound according to any embodiment described herein for use in medical therapy.

A number of assay systems can be used to determine if the compounds according to embodiments describe herein, are useful in the methods described above. For example, assays that measure if the compound induces the formation of a complex between the E3-ligase subunit and the target protein; assays that measure if the compound binds the E-3 ligase subunit, assays that measure if protein expression is decreased in a cell upon addition of the compounds and assays that measure cell viability. A non-limiting list of assays includes amplified luminescent proximity homogenous assay, homogeneous time fluorescence competition assays, Western blot assay, and CellTiter-Glo® Luminescent Cell Viability Assay.

These assays can be conducted with one or more positive control compounds-compounds that are known to induce an interaction between a ubiquitin E3 ligase and a target protein, leading to the degradation of the target protein. Examples of such positive control compounds, includes indisulam, E-7820; tasisulam and cloroquinoxaline sulfonamide (CQS).

Compounds of the invention were tested in the assays described in Examples 105-108.

### EXAMPLES

The following examples are given to illustrate the present subject matter. It should be understood, however, that the subject matter is not limited to the specific conditions or details described in these examples.

### Example 1: Preparation of 3-cyano-N-(3-cyano-4-methyl-1H-indol-7-yl)-5-fluorobenzenesulfonamide; Intermediate A

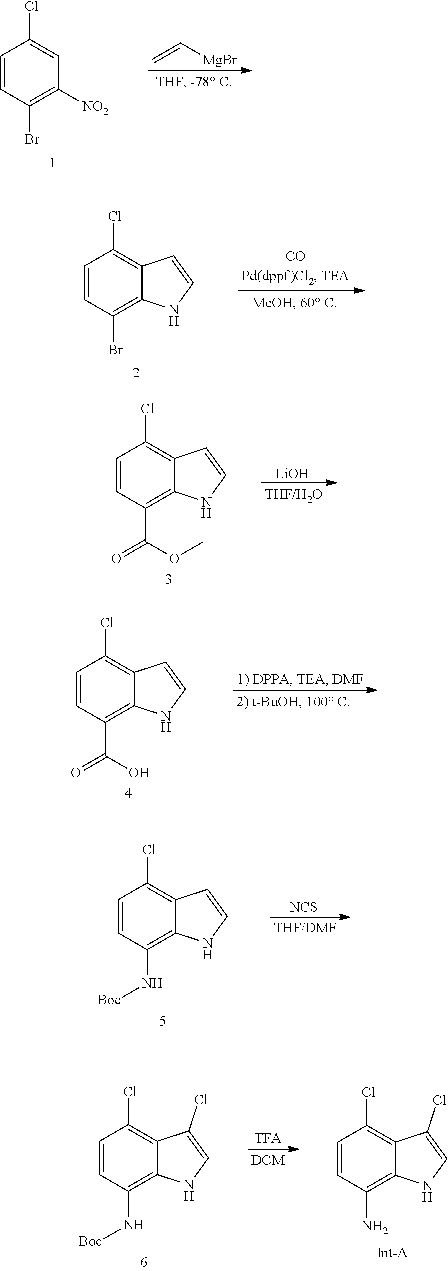

### 1. Preparation of 7-bromo-4-chloro-1H-indole

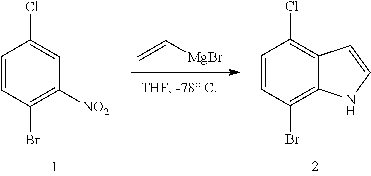

To a solution of 1-bromo-4-chloro-2-nitrobenzene (10.0 g, 42.29 mmol) in THF (100 mL) was added slowly bromo(vinyl)magnesium (211.5 mL, 211.46 mmol) at −78° C. under N₂ atmosphere. The mixture was stirred at −78° C. for 1 h. The mixture was quenched with sat. NH₄Cl and extracted with EA. The organic layer was concentrated and the residue was purified by chromatography column on silica gel to give 7-bromo-4-chloro-1H-indole (4.5 g, 45.8%) as a light yellow solid. LCMS (ESI): 230.0 [M+H]⁺.

### 2. Preparation of methyl 4-chloro-1H-indole-7-carboxylate

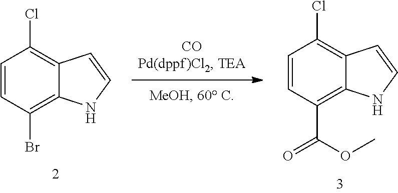

To a solution of 7-bromo-4-chloro-1H-indole (1.0 g, 4.34 mmol) in MeOH (10 mL), were added TEA (2 mL) and Pd(dppf)Cl₂ (320.0 mg, 0.43 mmol). The mixture was stirred at 60° C. under CO atmosphere (200 psi.) overnight. The mixture was filtered and the filtrate was concentrated. The residue was purified by chromatography column on silica gel eluting with PE/EA from 40/1 to 15/1 to afford methyl 4-chloro-1H-indole-7-carboxylate (542.0 mg, 59.6%) as a light-yellow solid. LCMS (ESI): 210 [M+H]⁺.

### 3. Preparation of 4-chloro-1H-indole-7-carboxylic acid

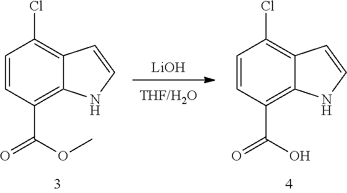

To a solution of methyl 4-chloro-1H-indole-7-carboxylate (200.0 mg, 0.95 mmol) in THF/H₂O (1:1, 4 mL), was added LiOH·H₂O (160.0 mg, 3.82 mmol). The mixture was stirred at rt for 3 h. The mixture was concentrated and diluted with H₂O (15 mL), then the mixture was acidified with 1N HCl aq. to pH-2. A solid precipitated was collected by filtration. The filtered cake was dissolved in EA, dried over anhydrous Na₂SO₄, then filtered and the filtrate was concentrated to give 4-chloro-1H-indole-7-carboxylic acid (207.0 mg, 98.0%) as a white solid. LCMS (ESI): 194 [M−H]⁻.

### 4. Preparation of tert-butyl (4-chloro-1H-indol-7-yl)carbamate

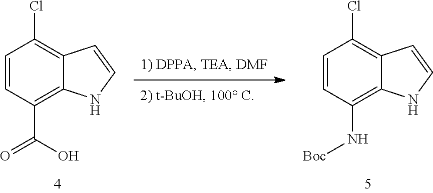

To a solution of 4-chloro-1H-indole-7-carboxylic acid (100.0 mg, 0.51 mmol) in t-Butanol (5 mL) was added TEA (207.6 mg, 207.6 mmol) and DPPA (169 mg, 0.61 mmol) under N₂ atmosphere, the mixture was stirred at 100° C. overnight. The reaction mixture was diluted with EA (10 mL). The mixture was washed with 1N HCl aq. (10 mL), water (10 mL) and brine (10 mL). The organic phase was dried over Na₂SO₄ and concentrated to give a residue, which was purified by column chromatography on silica gel eluting with PE/EA from 20/1 to 8/1 to afford tert-butyl N-(4-chloro-1H-indol-7-yl)carbamate (41.0 mg, 0.15 mmol, yield: 29.41%) as a white solid. LCMS (ESI): 267 [M+H]⁺.

### 5. Preparation of tert-butyl (3,4-dichloro-1H-indol-7-yl)carbamate

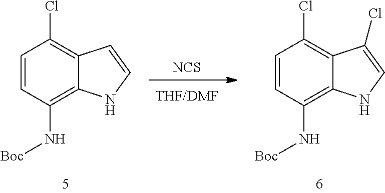 [SMILES: CC(C)(C)OC(=O)Nc1ccc(Cl)c2cc[nH]c12 ; "NCS" ; CC(C)(C)OC(=O)Nc1ccc(Cl)c2c(Cl)c[nH]c12]

To a solution of tert-butyl N-(4-chloro-1H-indol-7-yl)carbamate (500.0 mg, 1.88 mmol) in THF/DMF (10:1, 10 mL) was added NCS (262.9 mg, 1.97 mmol) at 0° C. The mixture was stirred at rt for 1 h. The reaction was quenched with saturated Na₂SO₃ solution (5 mL). The mixture was extracted with EA (20 mL). The organic layer was concentrated and purified by column chromatography on silica gel eluting with PE/EA from 20/1 to 8/1 to afford tert-butyl N-(3,4-dichloro-1H-indol-7-yl)carbamate (352.0 mg, 62.4%) as a white solid. LCMS (ESI): 301 [M+H]⁺.

### 6. Preparation of 3,4-dichloro-1H-indol-7-amine

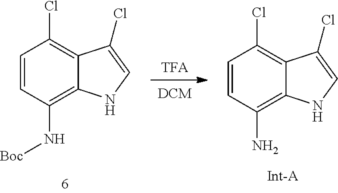

To a solution of tert-butyl N-(3,4-dichloro-1H-indol-7-yl)carbamate (2.4 g, 7.97 mmol) in DCM (20 mL) was added TFA (4 mL). The mixture was stirred at rt for 5 h. The mixture was concentrated and diluted with EA. The mixture was washed with sat. NaHCO₃. The organic layer was washed with brine, dried over Na₂SO₄, filtered, concentrated. The residue was recrystallized from DCM/hexane to afford 3,4-dichloro-1H-indol-7-amine (Int-A, 1.0 g, 65.6%) as a brown solid. LCMS (ESI): 200 [M+H]⁺. ¹H NMR (400 MHz, DMSO-d6) δ 11.20 (s, 1H), 7.50 (d, J=4.0 Hz, 1H), 6.78 (d, J=8.0 Hz, 1H), 6.32 (d, J=8.0 Hz, 1H), 5.28 (s, 2H).

### Example 2: Preparation of 7-amino-4-methyl-1H-indole-3-carbonitrile; Intermediate B

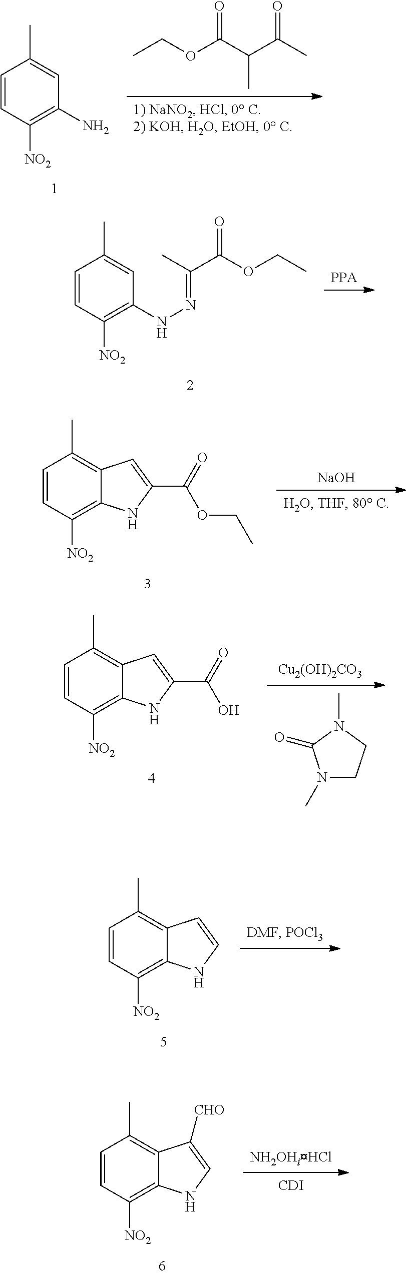

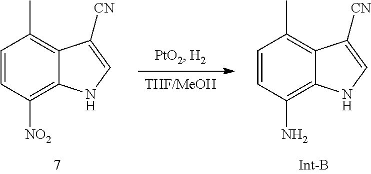

### 1. Preparation of ethyl (E)-2-(2-(5-methyl-2-nitrophenyl)hydrazono)propanoate

A mixture of 5-methyl-2-nitroaniline (20.0 g, 131.45 mmol) in con. HCl/H₂O (v/v=1/1, 90 mL) was stirred at −20° C. for 30 min. To the mixture was added dropwise NaNO₂ (10.9 g, 157.74 mmol) in water (20 mL). Then the mixture was stirred at 0° C. for 30 min to obtain solution A. To a solution of ethyl 2-methyl-3-oxobutanoate (19.9 g, 138.0 mmol) in ethanol (96 mL) was added KOH (36.81 g, 657.25 mmol) in H₂O (192 mL) at −20° C. The mixture was stirred at 0° C. for 30 min to obtain solution B. Solution B was added dropwise to solution A at −20° C. over 1 h with stirring. The mixture was stirred at 0° C. for another 30 min. The mixture was acidified to pH=1 with con. HCl. The resulting precipitate was collected by filtration, washed with water and dried under reduced pressure. A mixed solution of MTBE and hexane (1/2) was added thereto and crystals were collected by filtration. The filtered cake was dried by lyophilization to give ethyl (E)-2-(2-(5-methyl-2-nitrophenyl)hydrazono)propanoate (29.0 g, yield: 83.2%) as a yellow solid. LCMS (ESI): 266 [M+H]⁺.

### 2. Preparation of ethyl 4-methyl-7-nitro-1H-indole-2-carboxylate

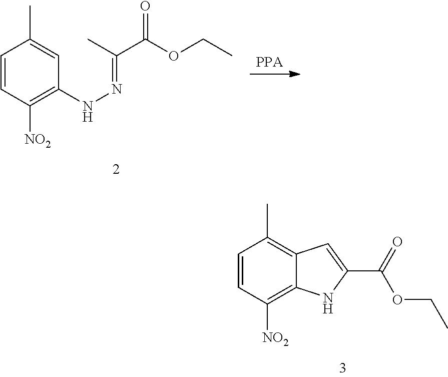

A mixture of ethyl (E)-2-(2-(5-methyl-2-nitrophenyl)hydrazono)propanoate (35.0 g, 132.08 mmol) in PPA (350 g) was stirred at 90° C. overnight. The mixture was cooled down to rt and dropped into sufficient water at rt slowly. The mixture was stirred at rt for 2 h and then extracted with EA (500 mL×2). The combined organic layer was concentrated to give a crude product, which was purified by column chromatography on silica gel to give ethyl 4-methyl-7-nitro-1H-indole-2-carboxylate (20.0 g, yield: 60.8%) as a yellow solid. LCMS (ESI): 249 [M+H]⁺. ¹H NMR (400 MHz, DMSO-d6) δ 11.29 (s, 1H), 8.20 (d, J=8.1 Hz, 1H), 7.52 (d, J=2.0 Hz, 1H), 7.17 (d, J=8.2, 1H), 4.39 (q, J=7.1 Hz, 2H), 2.66 (s, 3H), 1.37 (t, J=7.1 Hz, 3H).

### 3. Preparation of 4-methyl-7-nitro-1H-indole-2-carboxylic acid

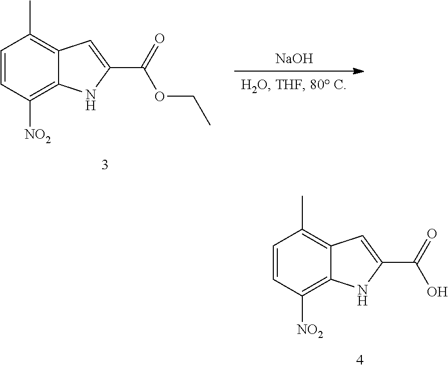 [SMILES: CCOC(=O)c1cc2c(C)ccc([N+](=O)[O-])c2[nH]1 ; "NaOH" ; "H2O, THF, 80° C." ; Cc1ccc([N+](=O)[O-])c2[nH]c(C(=O)O)cc12]

To a solution of ethyl 4-methyl-7-nitro-1H-indole-2-carboxylate (20.0 g, 80.32 mmol) in THF (300 mL) was added NaOH (9.6 g, 240.96 mmol) in water (250 mL). The mixture was stirred at 80° C. for 30 min. The mixture was concentrated under reduced pressure to remove the solvent. The residue was acidified to pH=1 with 5 N aqueous HCl. The mixture was extracted with DCM (300 mL×2). The combined organic layer was concentrated to give a crude product, which was purified by column chromatography on silica gel eluting with DCM/MeOH from 30/1 to 8/1 to give 4-methyl-7-nitro-1H-indole-2-carboxylic acid (10.0 g, yield: 56.3%) as a yellow solid. LCMS (ESI): 221 [M+H]⁺. ¹H NMR (400 MHz, DMSO-d6) δ 11.12 (s, 1H), 8.25 (d, J=8.1 Hz, 1H), 7.52 (d, J=2.1 Hz, 1H), 7.22 (d, J=8.2 Hz, 1H), 2.72 (s, 3H).

### 4. Preparation of 4-methyl-7-nitro-1H-indole

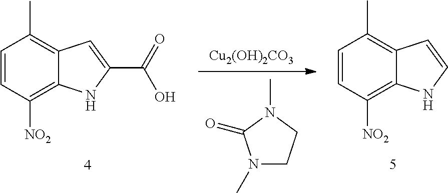

To a solution of 4-methyl-7-nitro-1H-indole-2-carboxylic acid (10.0 g, 45.25 mmol) in 1,3-dimethylimidazolidin-2-one (70 mL) was added Cu₂(OH)₂CO₃ (994.4 mg, 4.52 mmol). The reaction mixture was stirred at 170° C. for 6 h. The mixture was cooled to rt and diluted with water (150 mL) and EA (150 mL). The mixture was filtered with Celite. The filtrate was separated and the organic layers was washed with brine, dried over with Na₂SO₄, and concentrated to give a crude product, which was purified by column chromatography on silica gel to give 4-methyl-7-nitro-1H-indole (4.0 g, yield: 49.9%) as a yellow solid. LCMS (ESI): 177 [M+H]⁺. ¹H NMR (400 MHz, DMSO-d6) δ 11.88 (s, 1H), 8.02 (d, J=8.2 Hz, 1H), 7.50 (d, J=3.1 Hz, 1H), 7.05 (d, J=8.2 Hz, 1H), 6.76 (d, J=3.1 Hz, 1H), 2.61 (s, 3H).

### 5. Preparation of 4-methyl-7-nitro-1H-indole-3-carbaldehyde

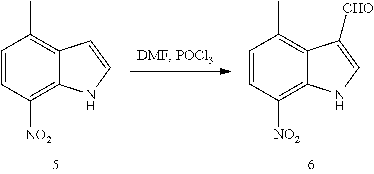

POCl₃ (1.12 mL, 12.02 mmol) was added to DMF (9 mL) at 0° C. in nitrogen atmosphere. The mixture was stirred at rt for 30 min to give solution A. Solution A was added to a 4-methyl-7-nitro-1H-indole (1.5 g, 8.52 mmol) in DMF (5 mL). The mixture was stirred at 50° C. for 4 h. The mixture was cooled to rt and dropped to sat.aqueous Na₂CO₃ (250 mL). The mixture was stirred at rt for 30 min and extracted with EA (250 mL×3). The combined organic layer was washed with water and brine, dried over Na₂SO₄, filtered and then concentrated to give a residue. A mixed solution of MTBE and hexane (1:5) was added to the residue and the crystals were collected by filtration to give 4-methyl-7-nitro-1H-indole-3-carbaldehyde (1.6 g, yield: 91.6%) as a yellow solid. LCMS (ESI): 205 [M+H]⁺. ¹H NMR (400 MHz, DMSO-d6) δ 12.72 (s, 1H), 10.04 (s, 1H), 8.41 (s, 1H), 8.14 (d, J=8.3 Hz, 1H), 7.28-7.17 (m, 1H), 2.92 (s, 3H).

### 6. Preparation of 4-methyl-7-nitro-1H-indole-3-carbonitrile

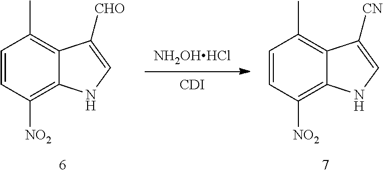

To a solution of 4-methyl-7-nitro-1H-indole-3-carbaldehyde (1.6 g, 7.80 mmol) in DMF (25 mL) were added NH₂OH·HCl (636.5 mg, 9.36 mmol) and pyridine (739.4 mg, 9.36 mmol). The mixture was stirred at 60° C. for 40 min. CDI (6.64 g, 40.93 mmol) was added while cooling in an iced-bath. The mixture was further heated and stirred at 60° C. for 30 min. TEA (2.273 mL, 16.37 mmol) was added and the mixture was continued heating and stirring at 60° C. for 1 h. The mixture was cooled to rt and ice-water (100 mL) was added. The mixture was extracted with EA (100 mL×3). The combined organic layer was washed with water and brine, dried over NaSO₄, filtered and concentrated to give a residue. A mixed solution of MTBE and hexane (1:5) was added to the residue and the crystals were collected by filtration to give 4-methyl-7-nitro-1H-indole-3-carbonitrile (1.5 g, 93.2%). LCMS (ESI): 200.0 [M−H]⁻. ¹H NMR (400 MHz, DMSO-d6) δ 12.77 (s, 1H), 8.43 (s, 1H), 8.15 (d, J=8.2 Hz, 1H), 7.24 (d, J=8.2 Hz, 1H), 2.80 (s, 3H).

### 7. Preparation of 7-amino-4-methyl-1H-indole-3-carbonitrile

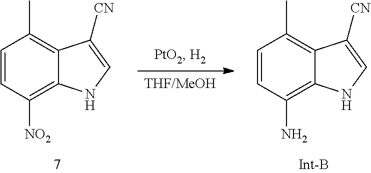

To a solution of 4-methyl-7-nitro-1H-indole-3-carbonitrile (1.1 g, 5.47 mmol) in THF/MeOH (1/1, 20 mL) was added PtO₂ (220 mg). The mixture was evaporated and backfilled with H₂ for three times. The mixture was stirred at rt for 4 h under H₂ atmosphere. The mixture was filtered and the filtrate was concentrated to give a crude product, which was purified by column chromatography on silica gel to give 7-amino-4-methyl-1H-indole-3-carbonitrile (Int-B, 832.0 mg, yield: 88.8%) as a white solid. LCMS (ESI): 172 [M+H]⁺. ¹H NMR (400 MHz, DMSO-d6) δ 11.72 (s, 1H), 8.12 (s, 1H), 6.72-6.60 (m, 1H), 6.36 (d, J=7.6 Hz, 1H), 5.08 (s, 2H), 2.49 (s, 3H).

### Example 3. Preparation of methyl ((4-(N-(3,4-dichloro-1H-indol-7-yl)sulfamoyl)phenyl)sulfonyl) glycinate; Compound 1

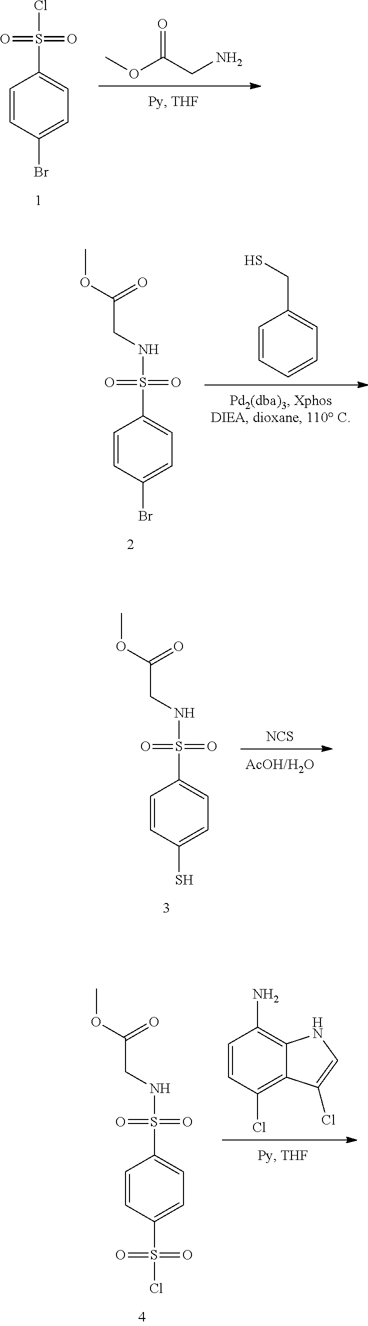

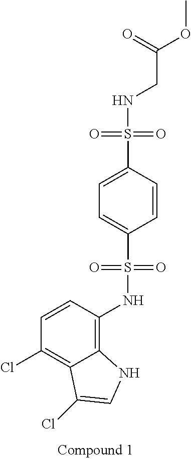 [SMILES: COC(=O)CNS(=O)(=O)c1ccc(S(=O)(=O)Nc2ccc(Cl)c3c(Cl)c[nH]c23)cc1]

### 1. Preparation of methyl ((4-bromophenyl)sulfonyl)glycinate

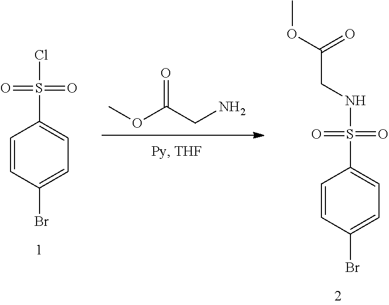

To a solution of methyl glycinate (173.1 mg, 1.17 mmol) and pyridine (378.7 mg, 3.75 mmol) in THF (10 mL) was added 4-bromobenzenesulfonyl chloride (318.8 mg, 1.25 mmol). The mixture was stirred at rt for 3 h until the starting material was consumed completely. The mixture was concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica eluting with PE/EA from 10/1 to 5/1 to give methyl ((4-bromophenyl)sulfonyl)glycinate (137.0 mg, yield 37.9%) as a white solid. LCMS (ESI) found: 308 [M+H]⁺.

### 2. Preparation of methyl ((4-(benzylthio)phenyl)sulfonyl)glycinate

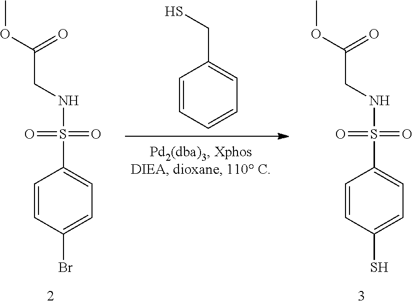 [SMILES: COC(=O)CNS(=O)(=O)c1ccc(Br)cc1 ; SCc1ccccc1 ; COC(=O)CNS(=O)(=O)c1ccc(S)cc1]

To a solution of methyl ((4-bromophenyl)sulfonyl)glycinate (137.0 mg, 0.46 mmol) and BnSH (57.0 mg, 0.46 mmol) in dioxane (2 mL) were added DIEA (148.3 mg, 1.15 mmol), Pd₂(dba)₃ (45.7 mg, 0.05 mmol) and Xantphos (57.8 mg, 0.10 mmol). The mixture was charged with N₂ for three times. The mixture was stirred at 110° C. for 3 hours under N₂ atmosphere until the starting material was consumed completely. The mixture was cooled to room temperature, filtered and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give methyl ((4-mercaptophenyl)sulfonyl)glycinate (142.0 mg, yield: 88.0%) as a yellow solid. LCMS (ESI) found: 262 [M+H]⁺.

### 3. Preparation of methyl ((4-(chlorosulfonyl)phenyl)sulfonyl)glycinate

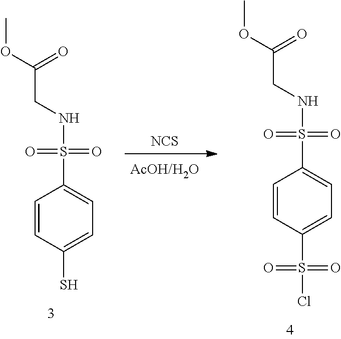 [SMILES: COC(=O)CNS(=O)(=O)c1ccc(S)cc1 ; "NCS" ; COC(=O)CNS(=O)(=O)c1ccc(S(=O)(=O)Cl)cc1]

To a solution of methyl ((4-mercaptophenyl)sulfonyl)glycinate (100.0 mg, 0.28 mmol) in AcOH/H₂O (9/1, 3 mL) was added NCS (157.6 mg, 1.18 mmol). The mixture was stirred at rt for 2 h. The reaction was quenched with aqueous Na₂S₂O₃ (15 mL) and extracted with DCM EA (20 mL×3). The combined organic layer was concentrated and purified by column chromatography on silica gel eluting with PE/EA from 15/1 to 5/1 to afford methyl ((4-(chlorosulfonyl)phenyl)sulfonyl)glycinate (61.7 mg, 67.9%) as a white solid. LCMS (ESI) found: 328 [M+H]⁺.

### 4. Preparation of methyl ((4-(N-(3,4-dichloro-1H-indol-7-yl)sulfamoyl) phenyl)sulfonyl)glycinate

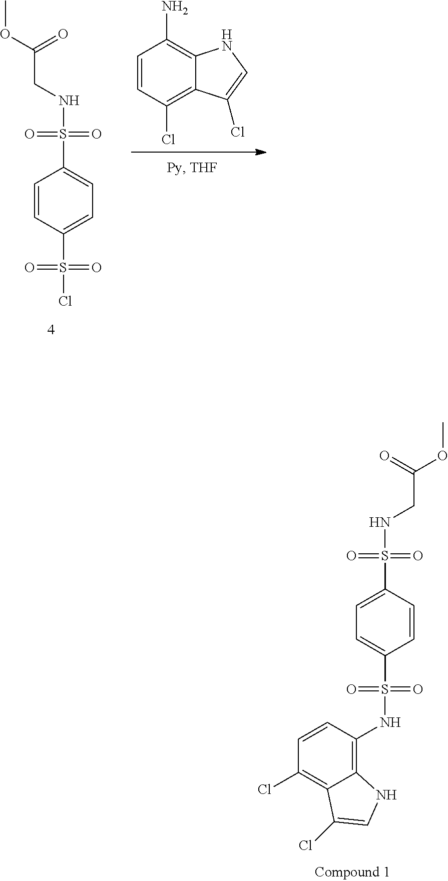 [SMILES: COC(=O)CNS(=O)(=O)c1ccc(S(=O)(=O)Cl)cc1 ; Nc1ccc(Cl)c2c(Cl)c[nH]c12 ; COC(=O)CNS(=O)(=O)c1ccc(S(=O)(=O)Nc2ccc(Cl)c3c(Cl)c[nH]c23)cc1]

To a solution of 3,4-dichloro-1H-indol-7-amine (24.8 mg, 0.12 mmol) and pyridine (28.4 mg, 0.36 mmol) in THF (5 mL) was added methyl ((4-(chlorosulfonyl)phenyl)sulfonyl)glycinate (39.2 mg, 0.12 mmol). The mixture was stirred at rt for 2 h. The mixture was concentrated to give a crude product, which was purified by chromatography column on silica gel eluting with PE/EA from 15/1 to 5/1 to afford methyl ((4-(N-(3,4-dichloro-1H-indol-7-yl)sulfamoyl)phenyl) sulfonyl)glycinate (Compound 1, 36.2 mg, 59.5%) as a white solid. LCMS (ESI) found: 492 [M+H]⁺. ¹H NMR (400 MHz, DMSO-d6) δ 11.49 (s, 1H), 10.23 (s, 1H), 8.48 (t, J=6.2 Hz, 1H), 7.91 (d, J=5.1 Hz, 4H), 7.56 (d, J=2.6 Hz, 1H), 6.94 (d, J=8.2 Hz, 1H), 6.62 (d, J=8.2 Hz, 1H), 3.77 (d, J=6.1 Hz, 2H), 3.41 (s, 3H).

### Example 4: Preparation of N¹-(3,4-dichloro-1H-indol-7-yl)-N⁴-(2-methoxyethyl)benzene-1,4-disulfonamide; Compound 2

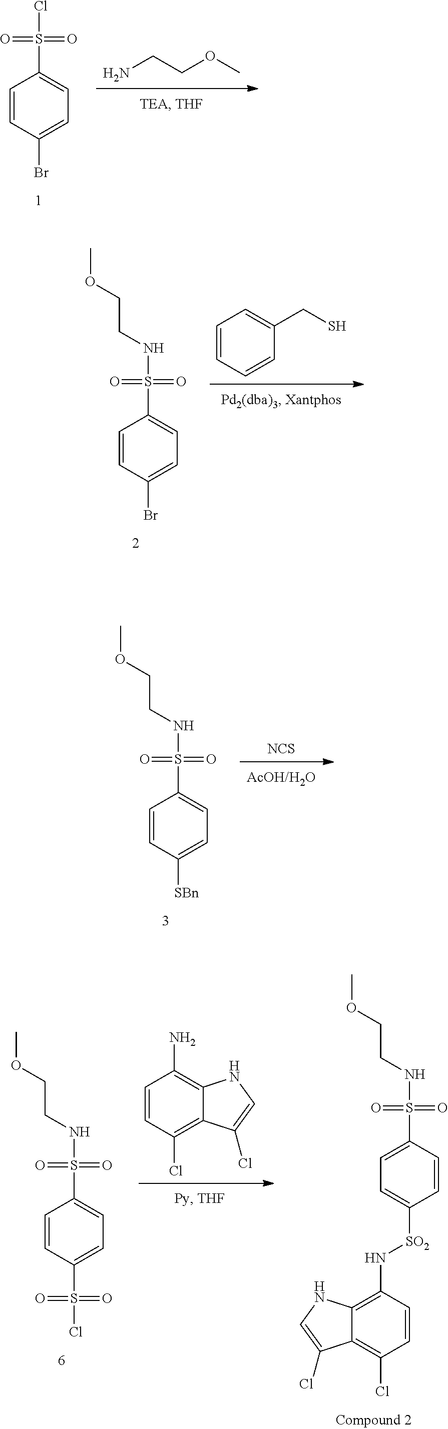

### 1. Preparation of 4-bromo-N-(2-methoxyethyl)benzenesulfonamide

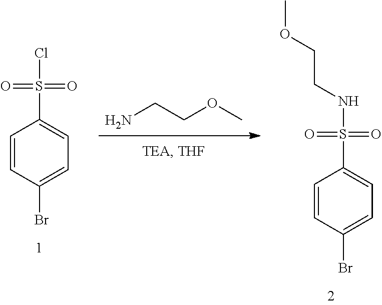

To a solution of 2-methoxyethan-1-amine (93.7 mg, 1.25 mmol) and TEA (378.7 mg, 3.75 mmol) in THF (10 mL) was added 4-bromobenzenesulfonyl chloride (318.8 mg, 1.25 mmol). The mixture was stirred at rt for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to afford a crude product, which was purified by column chromatography on silica eluting with PE/EA from 10/1 to 5/1 to give 4-bromo-N-(2-methoxyethyl)benzenesulfonamide (214.7 mg, yield 58.4%) as a white solid. LCMS (ESI) found: 294 [M+H]⁺.

### 2. Preparation of 4-(benzylthio)-N-(2-methoxyethyl)benzenesulfonamide

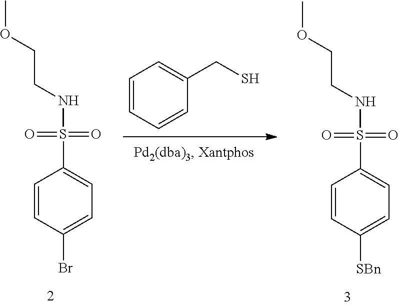

To a solution of 4-bromo-N-(2-methoxyethyl)benzenesulfonamide (188.1 mg, 0.64 mmol) and BnSH (79.4 mg, 0.64 mmol) in dioxane (2 mL) were added DIEA (206.4 mg, 1.60 mmol), Pd₂(dba)₃ (45.7 mg, 0.05 mmol) and Xantphos (57.8 mg, 0.10 mmol). The mixture was charged with N₂ for three times. Then the mixture was stirred at 90° C. for 3 h under N₂ atmosphere until the starting material was consumed completely. The mixture was cooled to room temperature, filtered and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 40/1 to 25/1 to give 4-(benzylthio)-N-(2-methoxyethyl)benzenesulfonamide (154.3 mg, yield: 71.3%) as a yellow solid. LCMS (ESI) found: 338 [M+H]⁺.

### 3. Preparation of 4-(N-(2-methoxyethyl)sulfamoyl)benzenesulfonyl chloride

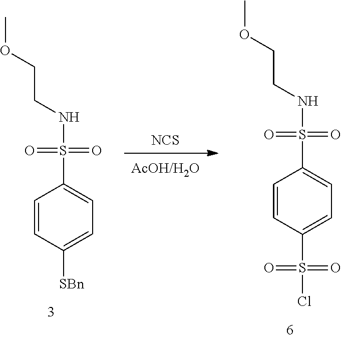

To a solution of 4-(benzylthio)-N-(2-methoxyethyl)benzenesulfonamide (100.0 mg, 0.31 mmol) in AcOH/H₂O (9/1, 3 mL) was added NCS (165.1 mg, 1.24 mmol). The mixture was stirred at rt for 2 h. The reaction was quenched with aqueous Na₂S₂O₃ (15 mL). The mixture was extracted with DCM (20 mL×3). The combined organic layer was concentrated to give a crude product, which was purified by column chromatography on silica gel eluting with PE/EA from 15/1 to 5/1 to afford 4-(N-(2-methoxyethyl)sulfamoyl)benzenesulfonyl chloride (70.0 mg, 71.0%) as a white solid. LCMS (ESI) found: 314 [M+H]⁺.

### 4. Preparation of N¹-(3,4-dichloro-1H-indol-7-yl)-N⁴-(2-methoxyethyl)benzene-1,4-disulfonamide

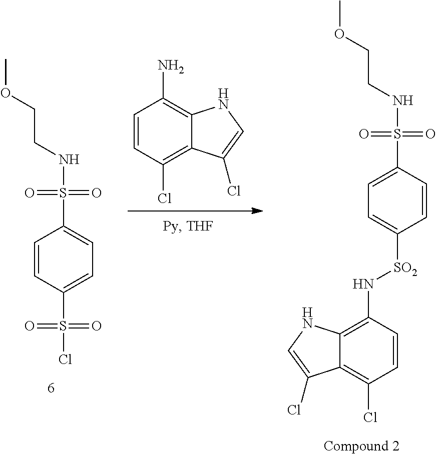 [SMILES: COCCNS(=O)(=O)c1ccc(S(=O)(=O)Cl)cc1 ; Nc1ccc(Cl)c2c(Cl)c[nH]c12 ; COCCNS(=O)(=O)c1ccc(S(=O)(=O)Nc2ccc(Cl)c3c(Cl)c[nH]c23)cc1]

To a solution of 3,4-dichloro-1H-indol-7-amine (45.0 mg, 0.22 mmol) and Pyridine (52.1 mg, 0.66 mmol) in THE (5 mL) was added 4-(N-(2-methoxyethyl)sulfamoyl)benzenesulfonyl chloride (70.0 mg, 0.22 mmol). The mixture was stirred at rt for 2 h. The mixture was concentrated to give a crude product, which was purified by column chromatography on silica gel eluting with PE/EA from 15/1 to 5/1 to afford N¹-(3,4-dichloro-1H-indol-7-yl)-N⁴-(2-methoxyethyl) benzene-1,4-disulfonamide (Compound 2, 96.0 mg, 90.0%) as a white solid. LCMS (ESI) found: 478 [M+H]⁺. ¹H NMR (400 MHz, DMSO-d6) δ 11.43 (s, 1H), 10.21 (s, 1H), 7.96 (t, J=5.8 Hz, 1H), 7.92-7.88 (m, 4H), 7.54 (s, 1H), 6.93 (d, J=8.1 Hz, 1H), 6.63 (d, J=8.2 Hz, 1H), 3.21 (t, J=5.5 Hz, 2H), 3.00 (s, 3H), 2.94-2.89 (m, 2H).

### Example 5: Preparation of N-(7-aminoheptyl)-N⁴-(3,4-dichloro-1H-indol-7-yl)benzene-1,4-disulfonamide; Compound 8

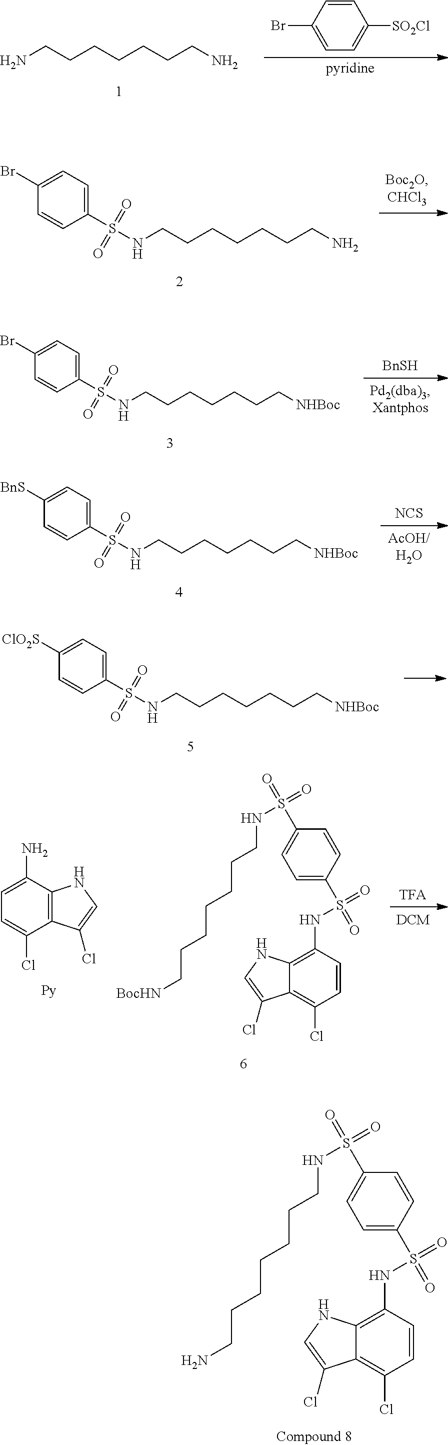

### 1. Preparation of N-(7-aminoheptyl)-4-bromobenzenesulfonamide

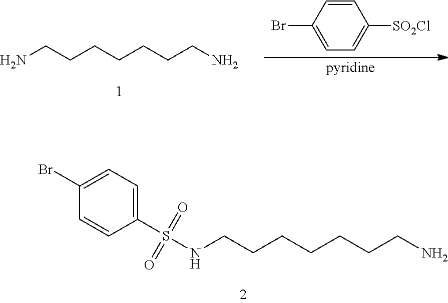

To a solution of heptane-1,7-diamine (2.9 mL, 19.21 mmol) in CHCl₃ (50 mL) were added pyridine (0.9 mL, 11.52 mmol) and 4-bromobenzenesulfonyl chloride (1.0 g, 3.84 mmol). The mixture was stirred at 0° C. for 2 h. TLC showed the reaction was completed. The mixture was concentrated. The residue was diluted with water (50 mL) and extracted with DCM (50 mL×3). The combined organic layer was concentrated to give a crude product, which was purified by column chromatography on silica gel eluting with PE/EA to give N-(7-aminoheptyl)-4-bromobenzenesulfonamide (0.8 g, 59.7%) as a solid. LCMS (ESI) found: 349 [M+H]⁺.

### 2. Preparation of tert-butyl (7-((4-bromophenyl)sulfonamido)heptyl)carbamate

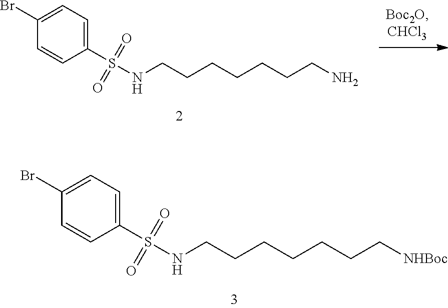

To a solution of N-(7-aminoheptyl)-4-bromobenzenesulfonamide (0.8 g, 2.29 mmol) in CHCl₃ (10 mL) were added TEA (1.0 mL, 6.87 mmol) and Boc₂O (0.5 mL, 2.52 mmol). The reaction mixture was stirred at rt for 1 h. TLC showed the reaction was completed. The mixture was concentrated under reduced pressure. The residue was diluted with water (30 mL) and extracted with EA (30 mL×3). The combined organic layer was concentrated to give a crude product, which was purified by column chromatography on silica gel eluting with PE/EA to give tert-butyl (7-((4-bromophenyl)sulfonamido)heptyl)carbamate (1.0 g, 97.1%) as a solid. LCMS (ESI) found: 449 [M+H]⁺.

### 3. Preparation of tert-butyl (7-((4-(benzylthio)phenyl)sulfonamido)heptyl)carbamate

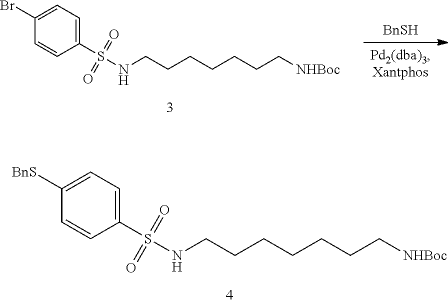

To a solution of N-(7-aminoheptyl)-4-bromobenzenesulfonamide (1.0 g, 2.22 mmol), BnSH (0.5 g, 4.45 mmol) and DIEA (1.1 mL, 6.67 mmol) in dioxane (10 mL) were added Pd₂(dba)₃ (0.4 g, 0.44 mmol) and Xantphos (0.5 g, 0.89 mmol) at rt. The mixture was charged with N₂ for three times and stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give tert-butyl (7-((4-bromophenyl)sulfonamido)heptyl)carbamate (700.0 mg, 63.8%) as a solid. LCMS (ESI) found: 493 [M+H]⁺.

### 4. Preparation of tert-butyl (7-((4-(chlorosulfonyl)phenyl)sulfonamido)heptyl)carbamate

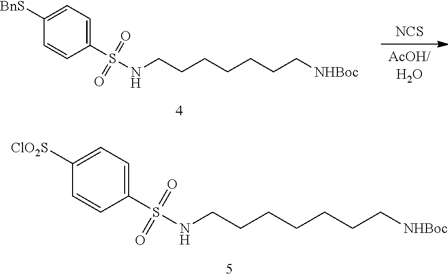

To a solution of tert-butyl (7-((4-bromophenyl)sulfonamido)heptyl)carbamate (270.0 mg, 0.55 mmol) in AcOH/H₂O (9/1, 5 mL) was added NCS (292.7 mg, 2.19 mmol). The mixture was stirred at rt for 2 h. Then the reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford tert-butyl (7-((4-(chlorosulfonyl)phenyl)sulfonamido)heptyl)carbamate (91.0 mg, 0.19 mmol, 35.41%) as a white solid. LCMS (ESI) found: 469 [M+1]⁺.

### 5. Preparation of tert-butyl (7-((4-(N-(3,4-dichloro-1H-indol-7-yl)sulfamoyl)phenyl)sulfonamido)heptyl)carbamate

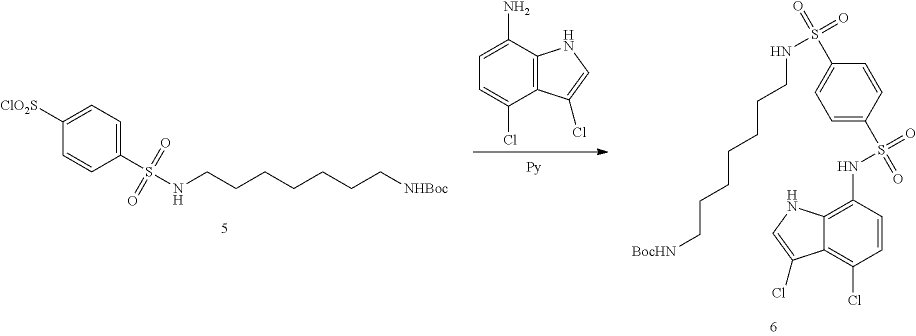 [SMILES: CC(C)(C)OC(=O)NCCCCCCCNS(=O)(=O)c1ccc(S(=O)(=O)Cl)cc1 ; Nc1ccc(Cl)c2c(Cl)c[nH]c12 ; CC(C)(C)OC(=O)NCCCCCCCNS(=O)(=O)c1ccc(S(=O)(=O)Nc2ccc(Cl)c3c(Cl)c[nH]c23)cc1]

To a solution of 3,4-dichloro-1H-indol-7-amine (17.0 mg, 0.08 mmol) in pyridine (20.1 mg, 0.25 mmol) was added tert-butyl (7-((4-(chlorosulfonyl)phenyl)sulfonamido)heptyl)carbamate (39.6 mg, 0.08 mmol). The mixture was stirred for 18 h at rt. After completion, the mixture was concentrated to afford a crude product, which was purified by column chromatography on silica gel eluting with PE/EA to give tert-butyl (7-((4-(N-(3,4-dichloro-1H-indol-7-yl)sulfamoyl)phenyl)sulfonamido)heptyl)carbamate (40.0 mg, 74.66%) as a solid. LCMS (ESI) found: 633 [M+H]⁺.

### 6. Preparation of N¹-(7-aminoheptyl)-N⁴-(3,4-dichloro-1H-indol-7-yl)benzene-1,4-disulfonamide

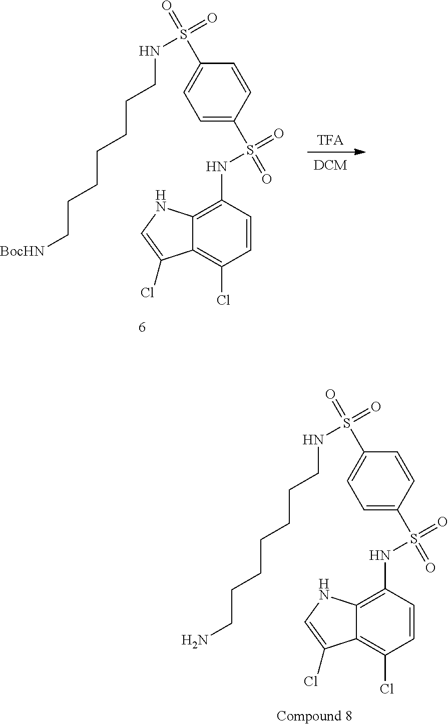

A solution of tert-butyl (7-((4-(N-(3,4-dichloro-1H-indol-7-yl)sulfamoyl)phenyl)sulfonamido)heptyl)carbamate (40.0 mg, 0.06 mmol) in DCM (0.5 mL) and TFA (0.5 mL) was stirred at rt for 1 h. LCMS showed the reaction was completed. The mixture was concentrated. The residue was basified with 10% aqueous NaHCO₃ (20 mL) and extracted with DCM (20 mL×3). The combined organic layer was purified by prep-HPLC to give N¹-(7-aminoheptyl)-N⁴-(3,4-dichloro-1H-indol-7-yl)benzene-1,4-disulfonamide (Compound 8, 15.0 mg, 44.5%) as a solid. LCMS (ESI) found: 533[M+H]⁺. ¹H NMR (400 MHz, DMSO-d6) δ 11.90 (s, 1H), 10.67 (s, 1H), 8.00 (s, 1H), 7.96 (d, J=8.5 Hz, 2H), 7.88 (d, J=8.5 Hz, 2H), 7.53 (d, J=2.8 Hz, 1H), 6.96 (d, J=8.1 Hz, 1H), 6.81 (d, J=8.1 Hz, 1H), 2.74-2.64 (m, 4H), 1.57-1.45 (m, 2H), 1.37-1.26 (m, 2H), 1.26-1.20 (m, 2H), 1.19-1.11 (m, 4H).

### Example 6: Preparation of N¹-(3,4-dichloro-1H-indol-7-yl)-N⁴-(tetrahydro-2H-pyran-4-yl)benzene-1,4-disulfonamide; Compound 28

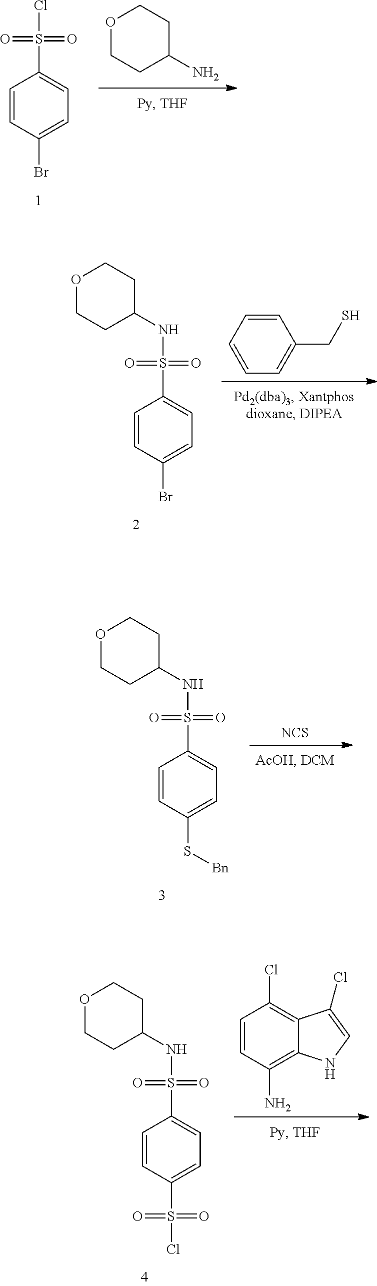

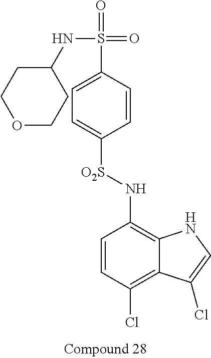 [SMILES: O=S(=O)(Nc1ccc(Cl)c2c(Cl)c[nH]c12)c1ccc(S(=O)(=O)NC2CCOCC2)cc1]

### 1. Preparation of 4-bromo-N-(tetrahydro-2H-pyran-4-yl)benzenesulfonamide

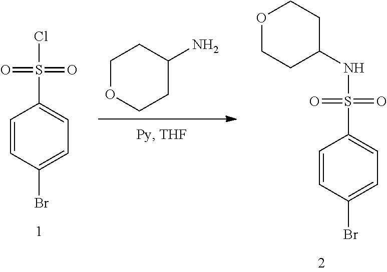

To a solution of tetrahydro-2H-pyran-4-amine (100.0 mg, 0.99 mmol) and pyridine (234.6 mg, 2.97 mmol) in THE (10 mL) was added 4-bromobenzenesulfonyl chloride (252.0 mg, 0.99 mmol). The mixture was at rt stirred for 3 h until the starting material was consumed completely. The mixture was concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica eluting with PE/EA from 10/1 to 5/1 to give 4-bromo-N-(tetrahydro-2H-pyran-4-yl)benzenesulfonamide (246.1 mg, yield 77.8%) as a white solid. LCMS (ESI) found: 320 [M+H]⁺.

### 2. Preparation of 4-(benzylthio)-N-(tetrahydro-2H-pyran-4-yl)benzenesulfonamide

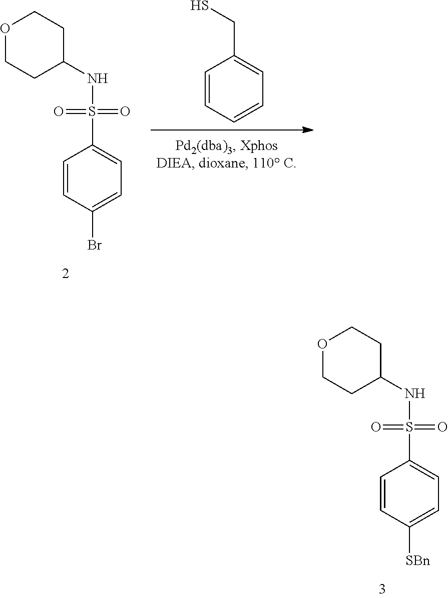

To a solution of 4-bromo-N-(tetrahydro-2H-pyran-4-yl)benzenesulfonamide (201.6 mg, 0.63 mmol) and BnSH (78.1 mg, 0.63 mmol) in dioxane (4 mL) were added DIEA (203.2 mg, 1.57 mmol), Pd₂(dba)₃ (54.9 mg, 0.06 mmol) and Xantphos (69.4 mg, 0.12 mmol). The mixture was charged with N₂ for three times. The mixture was stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give 4-(benzylthio)-N-(tetrahydro-2H-pyran-4-yl)benzenesulfonamide (203.0 mg, yield: 89.4%) as green oil. LCMS (ESI) found: 364 [M+H]⁺.

### 3. Preparation of 4-(N-(tetrahydro-2H-pyran-4-yl)sulfamoyl)benzenesulfonyl chloride

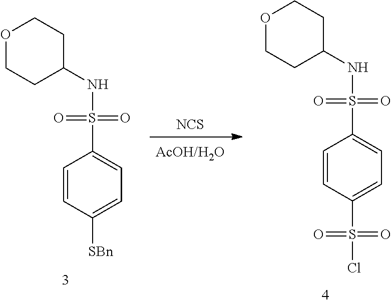

To a solution of 4-(benzylthio)-N-(tetrahydro-2H-pyran-4-yl)benzenesulfonamide (80.1 mg, 0.22 mmol) in AcOH/H₂O (9/1, 3 mL) was added NCS (123.8 mg, 0.92 mmol). The mixture was stirred at rt for 2 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 4-(N-(tetrahydro-2H-pyran-4-yl)sulfamoyl)benzenesulfonyl chloride (53.7 mg, 72.4%) as a white solid. LCMS (ESI) found: 340 [M+H]⁺.

### 4. Preparation of N¹-(3,4-dichloro-1H-indol-7-yl)-N⁴-(tetrahydro-2H-pyran-4-yl) benzene-1,4-disulfonamide

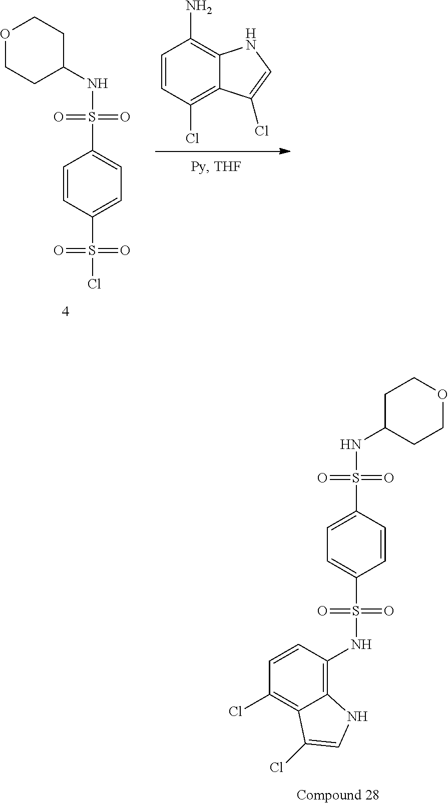

To a solution of 3,4-dichloro-1H-indol-7-amine (22.0 mg, 0.11 mmol) and Pyridine (26.1 mg, 0.33 mmol) in THE (5 mL) was added 4-(N-(tetrahydro-2H-pyran-4-yl)sulfamoyl) benzenesulfonyl chloride (37.5 mg, 0.11 mmol). The mixture was stirred at rt for 2 h. The mixture was concentrated to give a crude product, which was purified by column chromatography on silica gel eluting with PE/EA from 15/1 to 5/1 to afford N¹-(3,4-dichloro-1H-indol-7-yl)-N⁴-(tetrahydro-2H-pyran-4-yl)benzene-1,4-disulfonamide (Compound 28, 32.7 mg, 58.0%) as a white solid. LCMS (ESI) found: 504 [M+H]⁺. ¹H NMR (400 MHz, DMSO-d6) δ 11.44 (s, 1H), 10.19 (s, 1H), 8.01 (d, J=7.5 Hz, 1H), 7.94 (d, J=8.5 Hz, 2H), 7.88 (d, J=8.5 Hz, 2H), 6.91 (d, J=8.2 Hz, 1H), 6.59 (d, J=8.2 Hz, 1H), 3.73-3.67 (m, 2H), 3.23-3.16 (m, 3H), 1.40-1.25 (m, 4H).

### Example 7: Preparation of N¹-(3,4-dichloro-1H-indol-7-yl)-N⁴-(piperidin-4-yl)benzene-1,4-disulfonamide; Compound 29

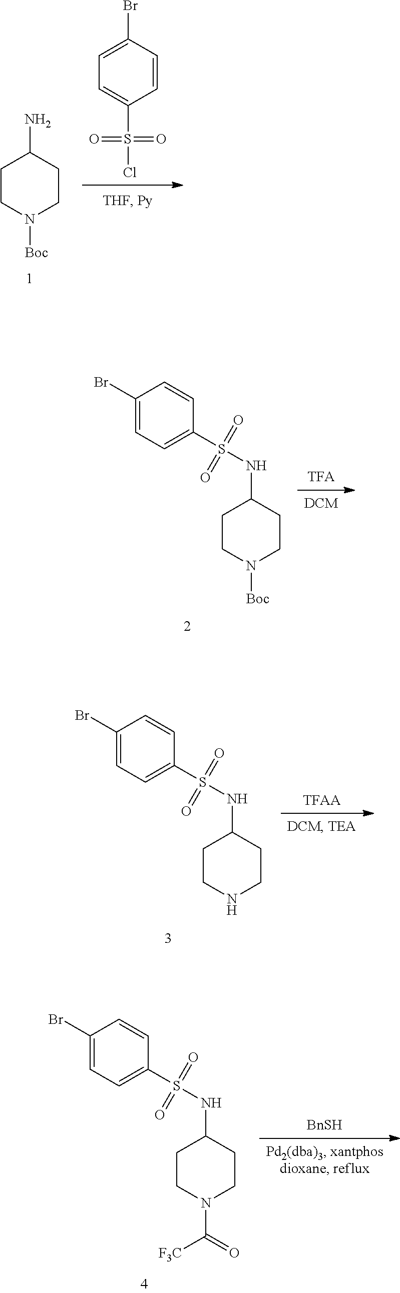

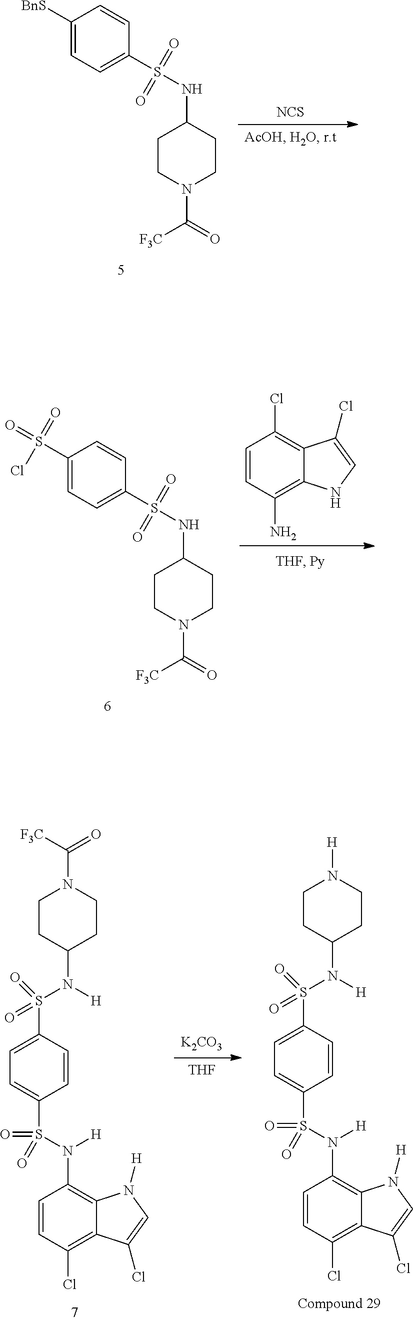

### 1. Preparation of tert-butyl 4-((4-bromophenyl)sulfonamido)piperidine-1-carboxylate

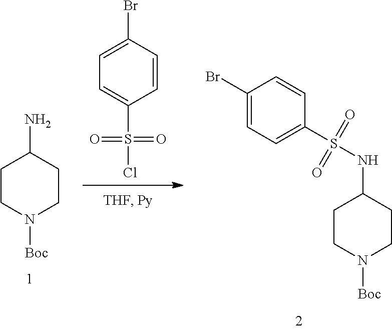 [SMILES: CC(C)(C)OC(=O)N1CCC(N)CC1 ; O=S(=O)(Cl)c1ccc(Br)cc1 ; CC(C)(C)OC(=O)N1CCC(NS(=O)(=O)c2ccc(Br)cc2)CC1]

To a solution of tert-butyl 4-aminopiperidine-1-carboxylate (500 mg, 2.50 mmol) in THF (5 mL) were added pyridine (592.5 mg, 7.50 mmol) and 4-bromobenzenesulfonyl chloride (63.5 mg, 2.50 mmol). The mixture was stirred at rt for 3 h until the starting material was consumed completely. The mixture was concentrated to give a residue, which was purified by flash column to give tert-butyl 4-((4-bromophenyl)sulfonamido)piperidine-1-carboxylate (550.0 mg, 52.5%) as white solid. LCMS (ESI) found: 419 [M+H]⁺.

### 2. Preparation of 4-bromo-N-(piperidin-4-yl)benzenesulfonamide

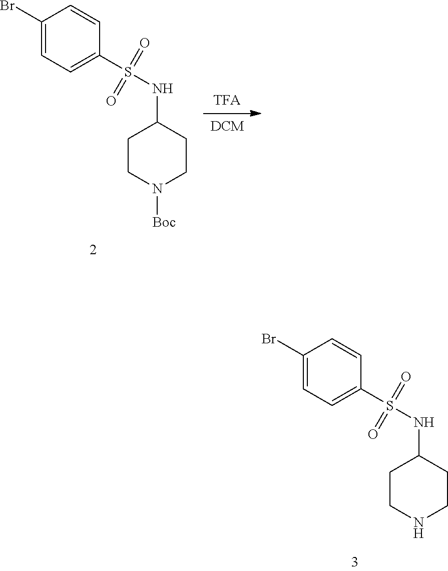

To a solution of tert-butyl 4-((4-bromophenyl)sulfonamido)piperidine-1-carboxylate (550.0 mg, 1.31 mmol) in DCM (10 mL) was added TFA (2 mL). The mixture was stirred at rt for 1 h until the starting material was consumed completely. The mixture was diluted with DCM (20 mL), basified with sat. Na₂CO₃ (20 mL) and washed with water (50 mL). The organic phase was dried over Na₂SO₄, filtered and concentrated to give a residue, which was purified by flash to give 4-bromo-N-(piperidin-4-yl)benzenesulfonamide (400.0 mg, 95.7%) as a yellow solid. LCMS (ESI) found: 319 [M+H]⁺.

### 3. Preparation of 4-bromo-N-(1-(2,2,2-trifluoroacetyl)piperidin-4-yl)benzenesulfonamide

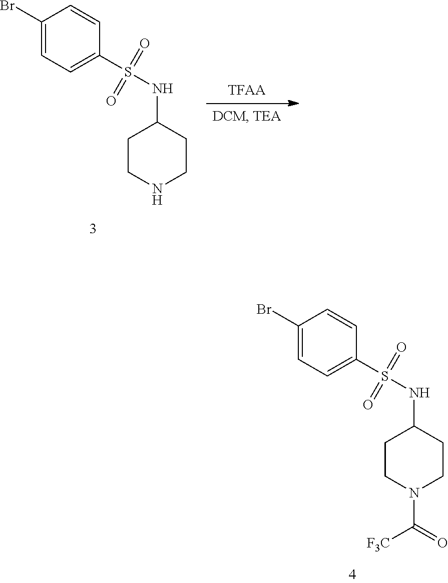 [SMILES: O=S(=O)(NC1CCNCC1)c1ccc(Br)cc1 ; "TFAA" ; "DCM, TEA" ; O=C(N1CCC(NS(=O)(=O)c2ccc(Br)cc2)CC1)C(F)(F)F]

To a solution of 4-bromo-N-(piperidin-4-yl)benzenesulfonamide (400.0 mg, 1.25 mmol) in DCM (10 mL) were added TEA (378.7 mg, 3.75 mmol) and TFAA (288.7 mg, 1.37 mmol). The mixture was stirred at rt for 1 h until the starting material was consumed completely. The reaction mixture was diluted with DCM (20 mL) and washed with water (20 mL). The organic layer was dried over Na₂SO₄, filtered and concentrated to give a crude product, which was purified by flash column to give 4-bromo-N-(1-(2,2,2-trifluoroacetyl)piperidin-4-yl)benzene sulfonamide (450.0 mg, 86.8%) as a yellow solid. LCMS (ESI) found: 415 [M+H]⁺.

### 4. Preparation of 4-(benzylthio)-N-(1-(2,2,2-trifluoroacetyl)piperidin-4-yl)benzene sulfonamide

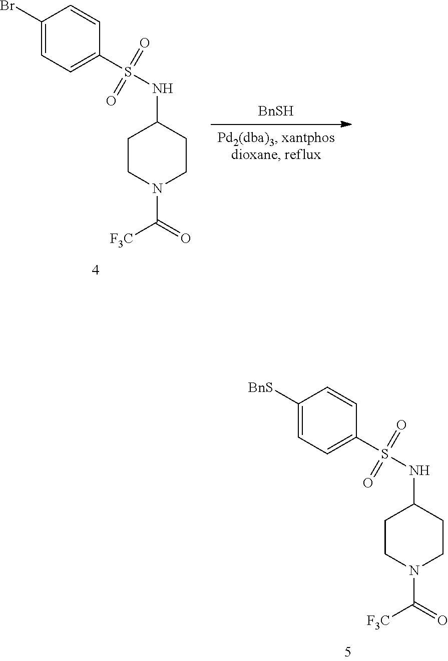

To a solution of 4-bromo-N-(1-(2,2,2-trifluoroacetyl)piperidin-4-yl)benzenesulfonamide (450.0 mg, 1.08 mmol) and BnSH (133.9 mg, 1.08 mmol) in dioxane (5 mL) were added DIEA (417.9 mg, 3.24 mmol), Pd₂(dba)₃ (100.7 mg, 0.11 mmol) and Xantphos (127.1 mg, 0.22 mmol). The mixture was evaporated and backfilled with N₂ for three times. The mixture was stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give 4-(benzylthio)-N-(1-(2,2,2-trifluoroacetyl)piperidin-4-yl) benzenesulfonamide (410.0 mg, 82.4%) as a yellow solid. LCMS (ESI) found: 459 [M+H]⁺.

### 5. Preparation of 4-(N-(1-(2,2,2-trifluoroacetyl)piperidin-4-yl)sulfamoyl)benzenesulfonyl chloride

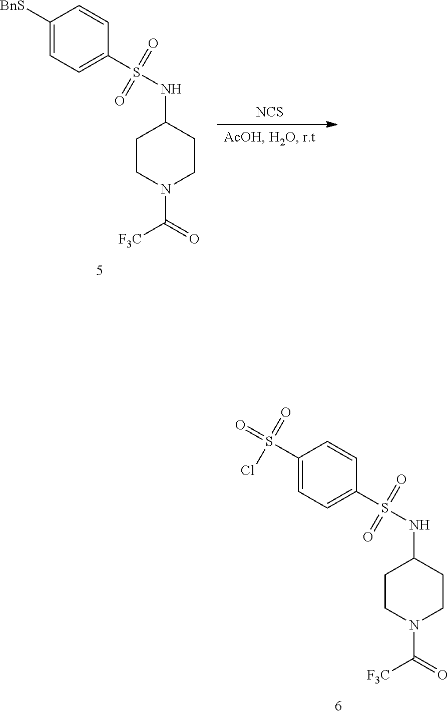

To a solution of 4-(benzylthio)-N-(1-(2,2,2-trifluoroacetyl)piperidin-4-yl)benzenesulfonamide (410.0 mg, 0.89 mmol) in AcOH/H₂O (9:1, 4.5 mL) was added NCS (504.9 mg, 3.74 mmol). The mixture was stirred at rt for 2 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to give 4-(N-(1-(2,2,2-trifluoroacetyl)piperidin-4-yl)sulfamoyl) benzenesulfonyl chloride (280.0 mg, 72.3%) as a white solid. LCMS (ESI) found: 435 [M+H]⁺.

### 6. Preparation of N¹-(3,4-dichloro-1H-indol-7-yl)-N⁴-(1-(2,2,2-trifluoroacetyl)piperidin-4-yl) benzene-1,4-disulfonamide

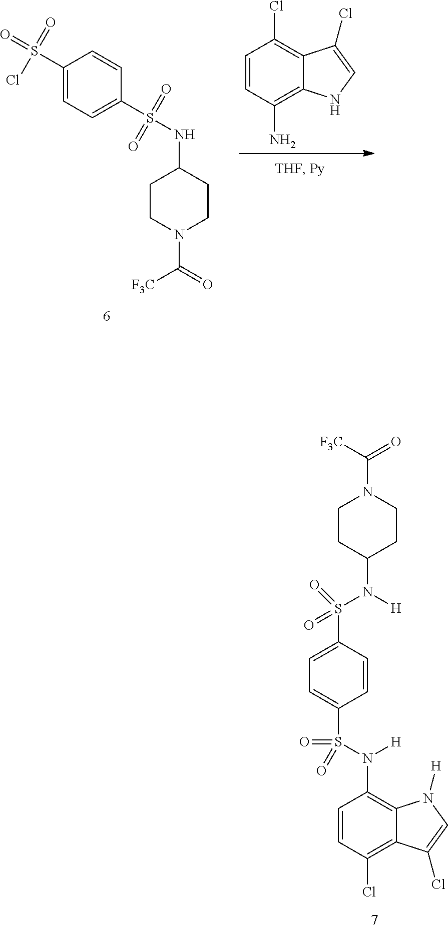 [SMILES: O=C(N1CCC(NS(=O)(=O)c2ccc(S(=O)(=O)Cl)cc2)CC1)C(F)(F)F ; Nc1ccc(Cl)c2c(Cl)c[nH]c12 ; O=C(N1CCC(NS(=O)(=O)c2ccc(S(=O)(=O)Nc3ccc(Cl)c4c(Cl)c[nH]c34)cc2)CC1)C(F)(F)F]

To a solution of 3,4-dichloro-1H-indol-7-amine (40.0 mg, 0.20 mmol) and pyridine (54.5 mg, 0.69 mmol) in THF (3 mL) was added 4-(N-(1-(2,2,2-trifluoroacetyl)piperidin-4-yl)sulfamoyl) benzenesulfonyl chloride (95.7 mg, 0.22 mmol). The mixture was stirred at rt for 18 h until the starting material was consumed completely. The reaction mixture was concentrated to give a residue, which was purified by prep-HPLC to give N¹-(3,4-dichloro-1H-indol-7-yl)-N⁴-(1-(2,2,2-trifluoroacetyl)piperidin-4-yl)benzene-1,4-disulfonamide (30.0 mg, 25.0%) as white solid. LCMS (ESI) found: 599 [M+H]⁺.

### 7. Preparation of N¹-(3,4-dichloro-1H-indol-7-yl)-N⁴-(piperidin-4-yl)benzene-1,4-disulfonamide

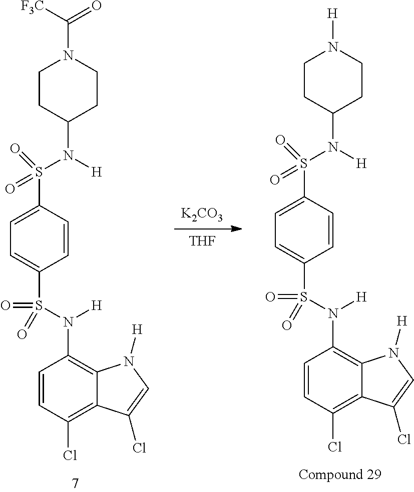 [SMILES: O=C(N1CCC(NS(=O)(=O)c2ccc(S(=O)(=O)Nc3ccc(Cl)c4c(Cl)c[nH]c34)cc2)CC1)C(F)(F)F ; "K2CO3" ; O=S(=O)(Nc1ccc(Cl)c2c(Cl)c[nH]c12)c1ccc(S(=O)(=O)NC2CCNCC2)cc1 ; "THF"]

To a solution of N¹-(3,4-dichloro-1H-indol-7-yl)-N₄-(1-(2,2,2-trifluoroacetyl) piperidin-4-yl)benzene-1,4-disulfonamide (30.0 mg, 0.050 mmol) in THF/H₂O (10 mL) was added K₂CO₃ (20.7 mg, 0.15 mmol). The mixture was stirred at 70° C. for 18 h until the starting material was consumed completely. The mixture was cooled to rt and filtered. The filtrated was concentrated to give a residue, which was purified by prep-HPLC to give N¹-(3,4-dichloro-1H-indol-7-yl)-N⁴-(piperidin-4-yl)benzene-1,4-disulfonamide (C, 11.3 mg, 44.9%) as a white solid (HCOOH salt). LCMS (ESI) found: 503 [M+H]⁺. ¹H NMR (400 MHz, DMSO-d6) δ 11.28 (s, 1H), 8.20 (s, 0.9H), 8.01 (s, 1H), 7.90 (d, J=8.4 Hz, 2H), 7.80 (d, J=8.4 Hz, 2H), 7.22 (s, 1H), 6.67-6.61 (m, 2H), 3.27-3.25 (m, 1H), 3.13-3.09 (m, 2H), 2.86-2.80 (m, 2H), 1.70-1.60 (m, 2H), 1.51-1.40 (m, 2H).

### Example 8: Preparation of N¹-(3,4-dichloro-1H-indol-7-yl)-N⁴-(piperidin-4-yl)-N⁴-propylbenzene-1,4-disulfonamide; Compound 30

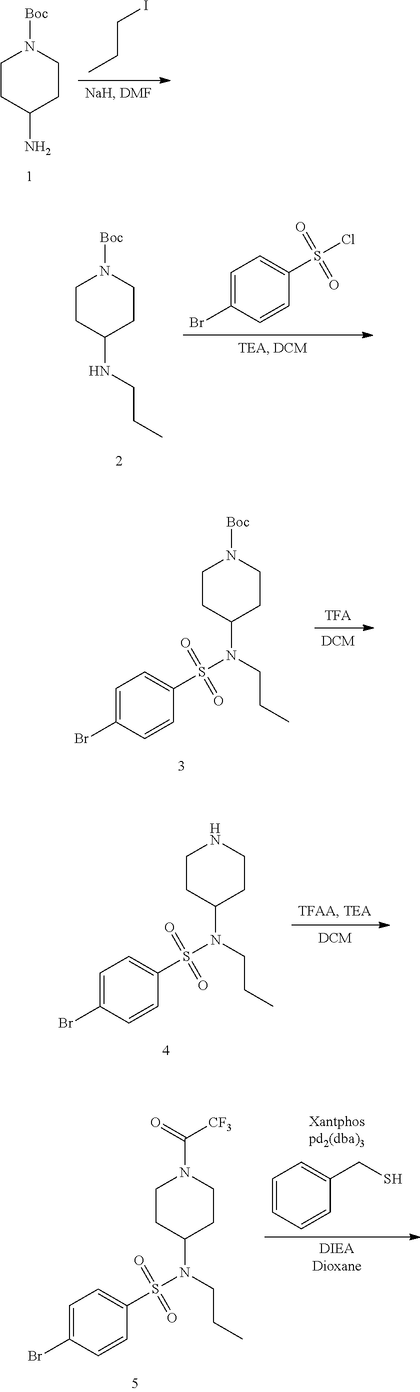

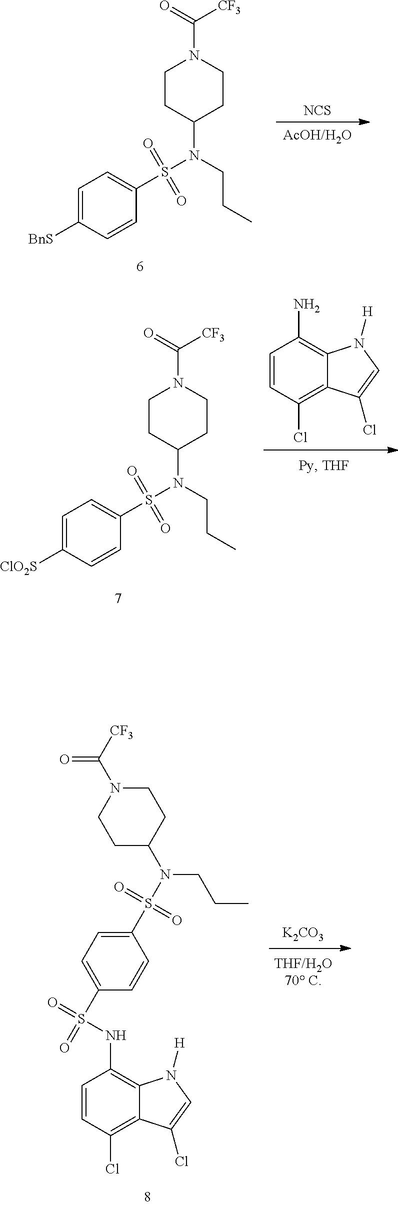

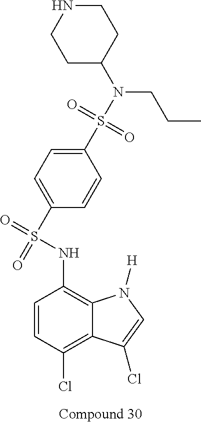 [SMILES: CCCN(C1CCNCC1)S(=O)(=O)c1ccc(S(=O)(=O)Nc2ccc(Cl)c3c(Cl)c[nH]c23)cc1]

### 1. Preparation of tert-butyl 4-(propylamino)piperidine-1-carboxylate

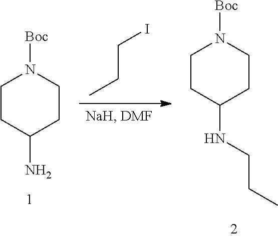

To a solution of tert-butyl 4-aminopiperidine-1-carboxylate (1.0 g, 4.99 mmol) in DMF (20 mL) was added NaH (60% in mineral oil, 0.2 g, 7.49 mmol) in portion wise at 0° C. The mixture was stirred at 0° C. for 20 min. Then 1-iodopropane (0.6 mL, 5.99 mmol) was added. The mixture was stirred at room temperature for 30 min. Then the mixture was quenched with sat. aqueous NH₄Cl (20 mL) and diluted with water (100 mL). The mixture was extracted with EA (100 mL×3). The combined organic layer was concentrated and purified by flash column to afford tert-butyl 4-(propylamino)piperidine-1-carboxylate (600.0 mg, 49.6%) as a colorless oil. LCMS (ESI) found: 243 [M+H]⁺.

### 2. Preparation of tert-butyl 4-((4-bromo-N-propylphenyl)sulfonamido)piperidine-1-carboxylate

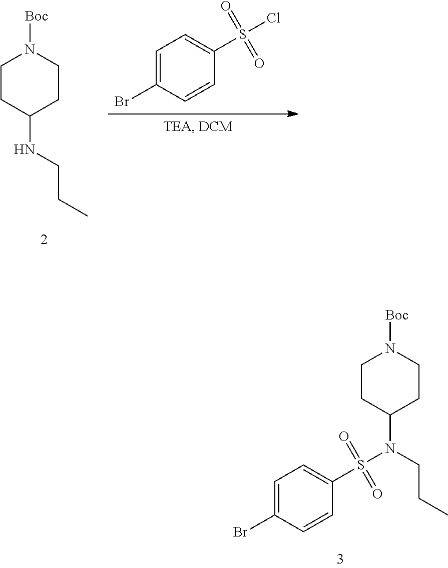

To a solution of tert-butyl 4-(propylamino)piperidine-1-carboxylate (600.0 mg, 2.48 mmol) and TEA (750.1 mg, 7.43 mmol) in DCM (10 mL) was added 4-bromobenzene-1-sulfonyl chloride (632.6 mg, 2.48 mmol). The mixture was stirred at room temperature for 30 min. The mixture was concentrated to give a residue, which was purified by flash column to afford tert-butyl 4-(N-propyl4-bromobenzenesulfonamido)piperidine-1-carboxylate (710.0 mg, 62.2%) as a white solid. LCMS (ESI) found: 461 [M+H]⁺.

### 3. Preparation of 4-bromo-N-(piperidin-4-yl)-N-propylbenzenesulfonamide

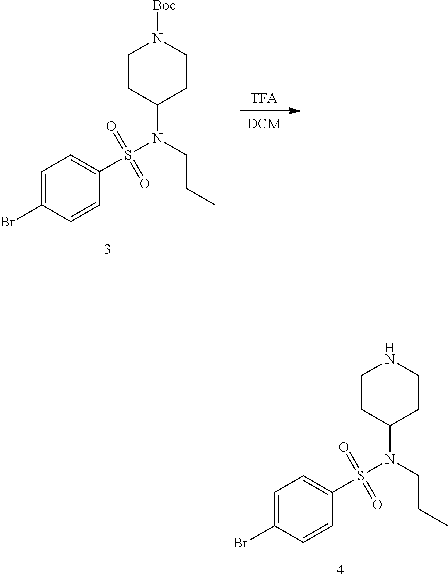

To a solution of tert-butyl 4-(N-propyl4-bromobenzenesulfonamido)piperidine-1-carboxylate (710.0 mg, 1.54 mmol) in DCM (2 mL) was added TFA (1 mL). The mixture was stirred at room temperature for 30 min. Then the reaction mixture was concentrated to give crude 4-bromo-N-(piperidin-4-yl)-N-propylbenzenesulfonamide (TFA salt), which was used for next step directly. LCMS (ESI) found: 361 [M+H]⁺.

### 4. Preparation of 4-bromo-N-propyl-N-(1-(2,2,2-trifluoroacetyl)piperidin-4-yl)benzenesulfonamide

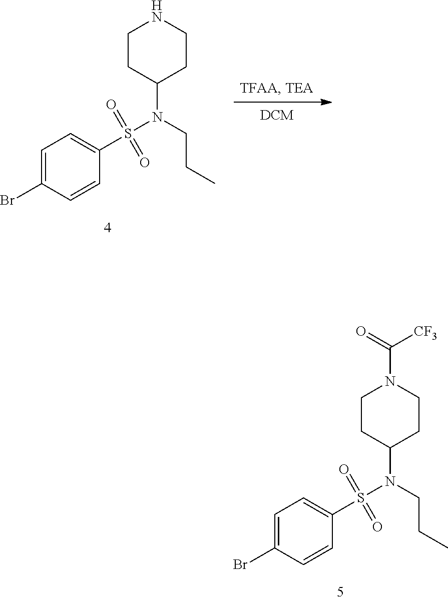

To a solution of 4-bromo-N-(piperidin-4-yl)-N-propylbenzene-1-sulfonamide in DCM (5 mL) were added TEA (466.6 mg, 4.62 mmol) and TFAA (380.1 mg, 1.85 mmol). The mixture was stirred at room temperature for 2 h. The mixture was poured into water (50 mL) and extracted with DCM (20 mL×3). The combined organic layer were concentrated and purified by flash column to afford 4-bromo-N-propyl-N-(1-(2,2,2-trifluoroacetyl)piperidin-4-yl) benzenesulfonamide (530.0 mg, 75.3%) as a white solid. LCMS (ESI) found: 457 [M+H]⁺.

### 5. Preparation of 4-(benzylthio)-N-propyl-N-(1-(2,2,2-trifluoroacetyl)piperidin-4-yl)benzenesulfonamide

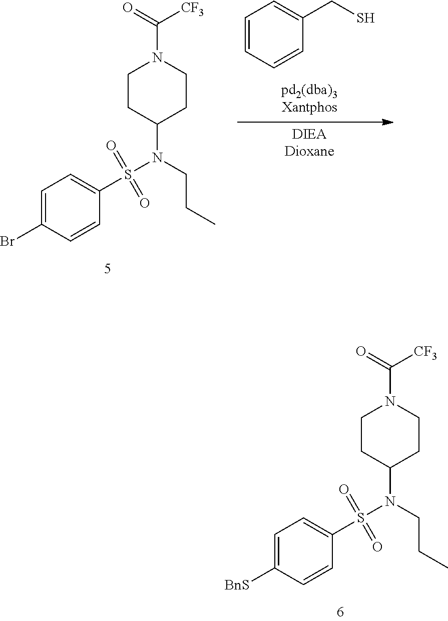

To a solution of 4-bromo-N-propyl-N-(1-(2,2,2-trifluoroacetyl)piperidin-4-yl)benzenesulfonamide (480.0 mg, 1.05 mmol) and BnSH (130.2 mg, 1.05 mmol) in dioxane (5 mL) were added TEA (212.1 mg, 2.10 mmol), Pd₂(dba)₃ (100.8 mg, 0.11 mmol), and Xantphos (127.2 mg, 0.22 mmol). The mixture was evaporated and backfilled with N₂ for three times. The mixture was stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give 4-(benzylthio)-N-propyl-N-(1-(2,2,2-trifluoroacetyl)piperidin-4-yl)benzene sulfonamide (270.0 mg, 51.4%) as a yellow solid. LCMS (ESI) found: 501 [M+H]⁺.

### 6. Preparation of 4-(N-propyl-N-(1-(2,2,2-trifluoroacetyl)piperidin-4-yl) sulfamoyl)benzenesulfonyl chloride

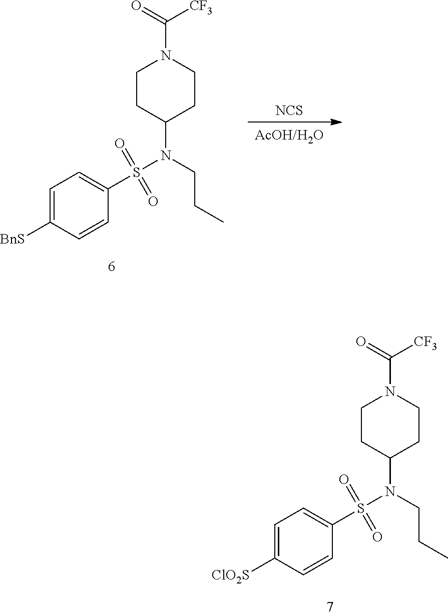

To a solution of 4-(benzylthio)-N-propyl-N-(1-(2,2,2-trifluoroacetyl)piperidin-4-yl)benzene sulfonamide (270.0 mg, 0.54 mmol) in AcOH/H₂O (9/1, 5 mL) was added NCS (360.1 mg, 2.70 mmol). The mixture was stirred at rt for 2 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 4-(N-propyl-N-(1-(2,2,2-trifluoroacetyl) piperidin-4-yl)sulfamoyl)benzenesulfonyl chloride (210.0 mg, 81.6%) as a white solid. LCMS (ESI) found: 477 [M+H]⁺.

### 7. Preparation of N¹-(3,4-dichloro-1H-indol-7-yl)-N⁴-propyl-N⁴-(1-(2,2,2-trifluoroacetyl)piperidin-4-yl)benzene-1,4-disulfonamide

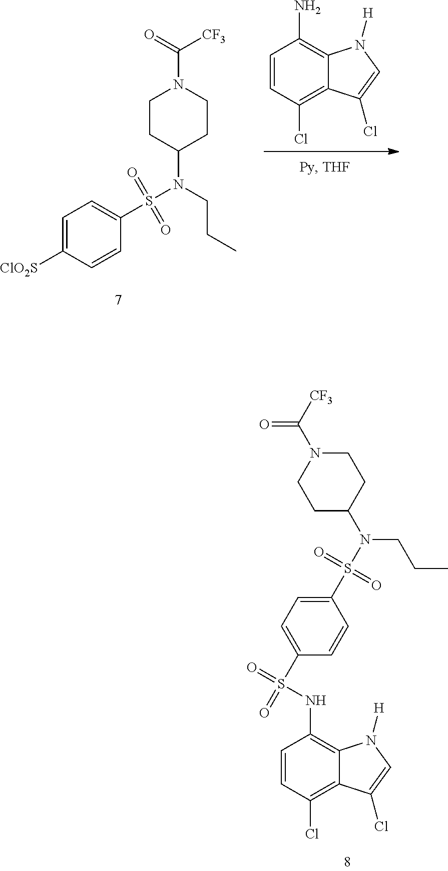 [SMILES: CCCN(C1CCN(C(=O)C(F)(F)F)CC1)S(=O)(=O)c1ccc(S(=O)(=O)Cl)cc1 ; Nc1ccc(Cl)c2c(Cl)c[nH]c12 ; CCCN(C1CCN(C(=O)C(F)(F)F)CC1)S(=O)(=O)c1ccc(S(=O)(=O)Nc2ccc(Cl)c3c(Cl)c[nH]c23)cc1]

To a solution of 3,4-dichloro-1-hydrogenio-1H-indol-7-amine (88.47 mg, 0.44 mmol) and Py (104.3 mg, 1.32 mmol) in THE (3 mL) was added 4-(N-propyl-N-(1-(2,2,2-trifluoroacetyl) piperidin-4-yl)sulfamoyl)benzenesulfonyl chloride (210.0 mg, 0.44 mmol). The mixture was stirred at room temperature for 2 h. The mixture was concentrated to give a residue, which was purified by flash column to afford N¹-(3,4-dichloro-1H-indol-7-yl)-N⁴-propyl-N⁴-(1-(2,2,2-trifluoroacetyl)piperidin-4-yl) benzene-1,4-disulfonamide (250.0 mg, 88.5%) as a white solid. LCMS (ESI) found: 641 [M+H]⁺.

### 8. Preparation of N¹-(3,4-dichloro-1H-indol-7-yl)-N⁴-(piperidin-4-yl)-N⁴-prop ylbenzene-1,4-disulfonamide

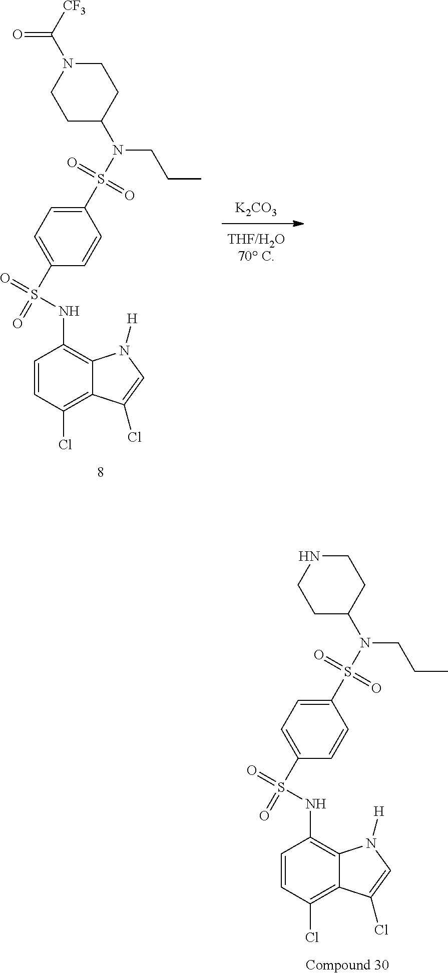

To a solution of N¹-(3,4-dichloro-1H-indol-7-yl)-N⁴-propyl-N⁴-(1-(2,2,2-trifluoroacetyl)piperidin-4-yl) benzene-1,4-disulfonamide (250.0 mg, 0.39 mmol) in THF/H₂O (4/1, 4 mL) was added K₂CO₃ (161.5 mg, 1.17 mmol). The mixture was stirred at 70° C. for 1 h. The mixture was cooled and filtered. The filtrate was concentrated and purified by prep-HPLC to afford N¹-(3,4-dichloro-1H-indol-7-yl)-N⁴-(piperidin-4-yl)-N⁴-propyl benzene-1,4-disulfonamide (HCOOH salt) (Compound 30, 50.0 mg, 23.5%) as a white solid. LCMS (ESI) found: 545 [M+H]⁺. ¹H NMR (400 MHz, DMSO-d6) δ 11.28 (s, 1H), 8.17 (s, 0.83H), 7.93-7.81 (m, 4H), 7.23 (s, 1H), 6.68-6.60 (m, 2H), 3.92-3.86 (m, 1H), 3.22-3.17 (m, 2H), 3.06-2.98 (m, 2H), 2.95-2.79 (m, 2H), 1.76 (q, J=7.4 Hz, 2H), 1.55-1.47 (m, 4H), 0.82 (t, J=7.4 Hz, 3H).

### Example 9: Preparation of N-(3,4-dichloro-1H-indol-7-yl)-4-(piperidin-1-ylsulfonyl)benzenesulfonamide; Compound 37

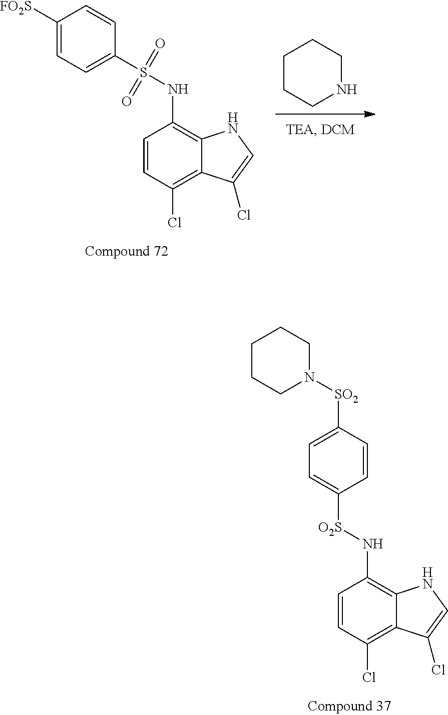

### 1. Preparation of N-(3,4-dichloro-1H-indol-7-yl)-4-(piperidin-1-ylsulfonyl)benzene sulfonamide

To a solution of 4-(N-(3,4-dichloro-1H-indol-7-yl)sulfamoyl)benzenesulfonyl fluoride (25.0 mg, 0.06 mmol) and TEA (19.2 mg, 0.19 mmol) in DCM (2 mL) was added piperidine (6.0 mg, 0.07 mmol). The mixture was stirred for 18 h at room temperature. The mixture was concentrated to give a crude product, which was purified by column chromatography on silica gel eluting with PE/EA to give N-(3,4-dichloro-1H-indol-7-yl)-4-(piperidin-1-ylsulfonyl)benzene sulfonamide (Compound 37, 8.9 mg, yield: 58.3%) as a white solid. LCMS (ESI) found: 459 [M+H]⁺. ¹H NMR (400 MHz, DMSO-d6) δ 11.38 (s, 1H), 10.20 (s, 1H), 7.90-7.83 (m, 4H), 7.53 (d, J=2.3 Hz, 1H), 6.95 (d, J=8.2 Hz, 1H), 6.64 (d, J=8.2 Hz, 1H), 2.88-2.80 (m, 4H), 1.52-1.46 (m, 4H), 1.38 (d, J=4.5 Hz, 2H).

### Example 10: Preparation of N-(3,4-dichloro-1H-indol-7-yl)-4-((4-hydroxypiperidin-1-yl)sulfonyl)benzene sulfonamide Compound 38

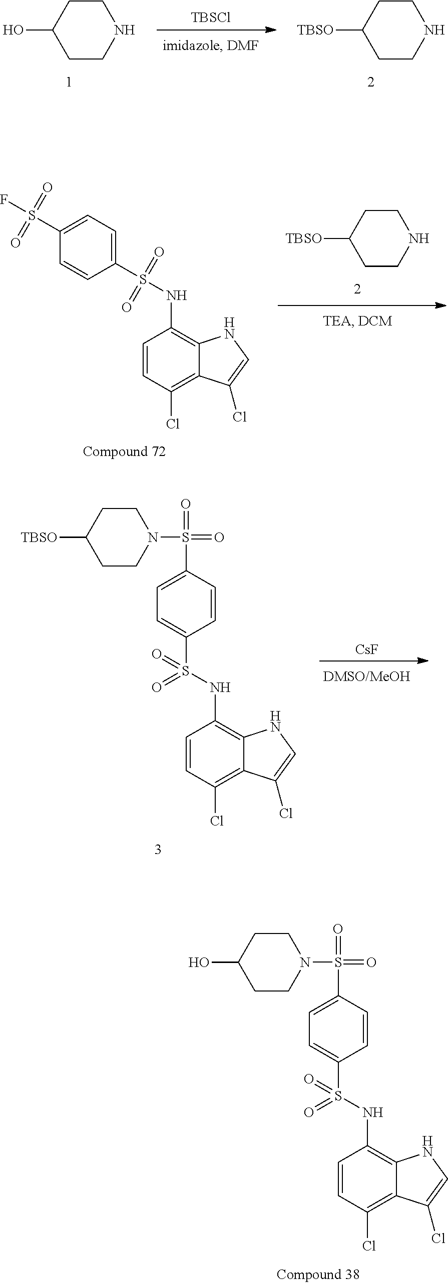

### 1. Preparation of 4-((tert-butyldimethylsilyl)oxy)piperidine

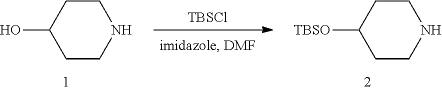 [SMILES: OC1CCNCC1 ; "TBSCl" ; CC(C)(C)[Si](C)(C)OC1CCNCC1]

To a solution of piperidin-4-ol (1.0 g, 9.90 mmol) and imidazole (1.0 g, 14.85 mmol) in DMF (10 mL) was added TBSCI (1.8 g, 11.88 mmol) at rt. The mixture was stirred for 18 h at room temperature. The mixture was concentrated and purified to afford a crude product, which was purified by column chromatography on silica gel eluting with PE/EA to give 4-((tert-butyldimethylsilyl)oxy)piperidine (810.0 mg, yield: 38.1%) as a white solid. LCMS (ESI) found: 216 [M+H]⁻.

### 2. Preparation of 4-((4-((tert-butyldimethylsilyl)oxy)piperidin-1-yl)sulfonyl)-N-(3,4-dichloro-1H-indol-7-yl)benzenesulfonamide

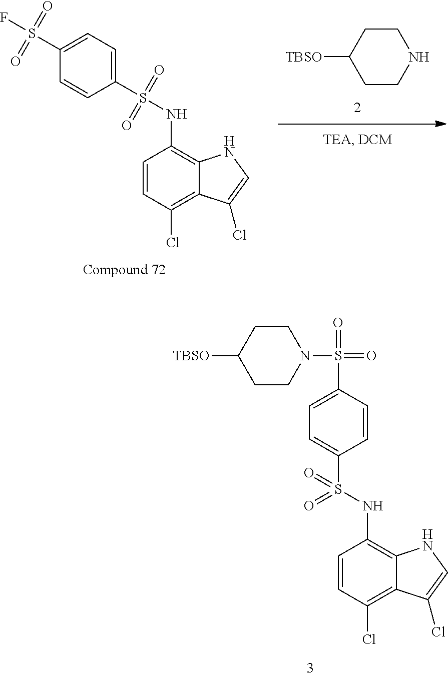 [SMILES: O=S(=O)(F)c1ccc(S(=O)(=O)Nc2ccc(Cl)c3c(Cl)c[nH]c23)cc1 ; CC(C)(C)[Si](C)(C)OC1CCNCC1 ; CC(C)(C)[Si](C)(C)OC1CCN(S(=O)(=O)c2ccc(S(=O)(=O)Nc3ccc(Cl)c4c(Cl)c[nH]c34)cc2)CC1]

To a solution of 4-((tert-butyldimethylsilyl)oxy)piperidine (25.3 mg, 0.12 mmol) and TEA (36.4 mg, 0.36 mmol) in DCM (2 mL) was added 4-(N-(3,4-dichloro-1H-indol-7-yl)sulfamoyl) benzenesulfonyl fluoride (50.0 mg, 0.12 mmol) at rt. The mixture was stirred for 18 h at room temperature. The mixture was concentrated and purified to afford the crude product, which was purified by column chromatography on silica gel eluting with PE/EA to give 4-((4-((tert-butyldimethylsilyl)oxy)piperidin-1-yl)sulfonyl)-N-(3,4-dichloro-1H-indol-7-yl)benzenesulfonamide (17.0 mg, yield: 22.9%) as a white solid. LCMS (ESI) found: 618 [M+H]⁺.

### 3. Preparation of N-(3,4-dichloro-1H-indol-7-yl)-4-((4-hydroxypiperidin-1-yl)sulfonyl) benzenesulfonamide

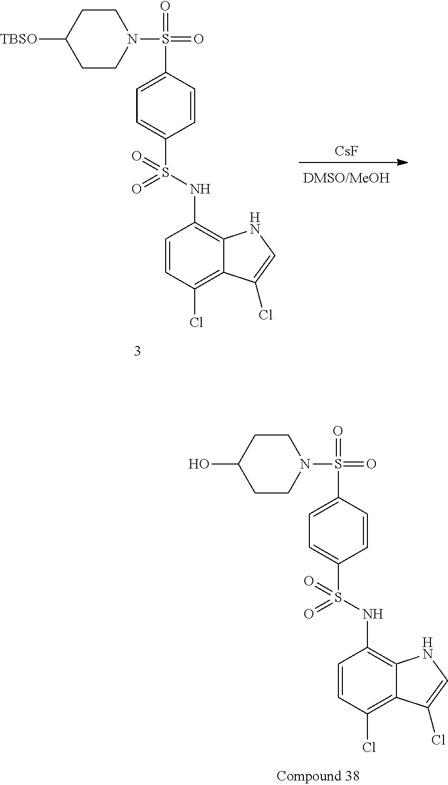 [SMILES: CC(C)(C)[Si](C)(C)OC1CCN(S(=O)(=O)c2ccc(S(=O)(=O)Nc3ccc(Cl)c4c(Cl)c[nH]c34)cc2)CC1 ; "CsF" ; O=S(=O)(Nc1ccc(Cl)c2c(Cl)c[nH]c12)c1ccc(S(=O)(=O)N2CCC(O)CC2)cc1]

To a solution of 4-((4-((tert-butyldimethylsilyl)oxy)piperidin-1-yl)sulfonyl)-N-(3,4-dichloro-1H-indol-7-yl)benzenesulfonamide (15.0 mg, 0.024 mmol) in DMSO/MeOH (100:1, 2 mL) was added CsF (10.9 mg, 0.072 mmol) at rt. The mixture was stirred for 18 h at room temperature. The mixture was purified by flash eluting with PE/EA to give N-(3,4-dichloro-1H-indol-7-yl)-4-((4-hydroxypiperidin-1-yl)sulfonyl) benzenesulfonamide (Compound 38, 9.2 mg, yield: 76.1%) as a white solid. LCMS (ESI) found: 504 [M+H]⁺. ¹H NMR (400 MHz, DMSO-d6) δ 11.49 (s, 1H), 10.20 (s, 1H), 7.91-7.87 (m, 4H), 7.57 (d, J=2.8 Hz, 1H), 6.97 (d, J=8.1 Hz, 1H), 6.51 (d, J=8.1 Hz, 1H), 4.76 (s, 1H), 3.57 (s, 1H), 3.21-3.14 (m, 2H), 2.73-2.68 (m, 2H), 1.76-1.70 (m, 2H), 1.47-1.36 (m, 2H).

### Example 11: Preparation of N-(3,4-dichloro-1H-indol-7-yl)-3-(piperazin-1-ylsulfonyl)benzene sulfonamide: Compound 41

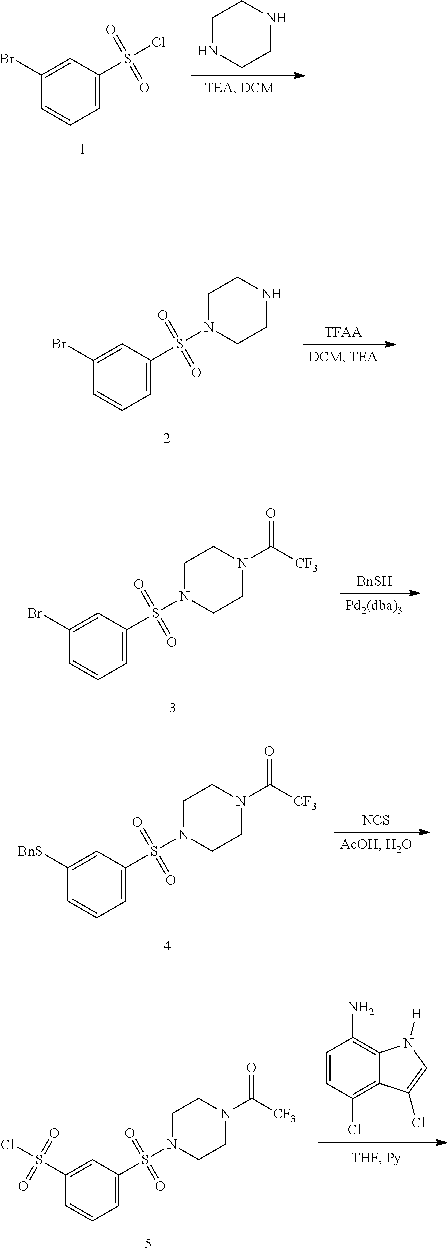

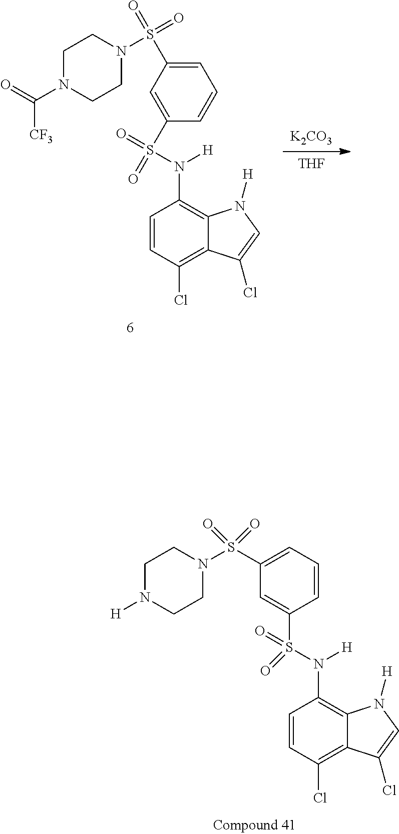 [SMILES: O=C(N1CCN(S(=O)(=O)c2cccc(S(=O)(=O)Nc3ccc(Cl)c4c(Cl)c[nH]c34)c2)CC1)C(F)(F)F ; "K2CO3" ; "THF" ; O=S(=O)(Nc1ccc(Cl)c2c(Cl)c[nH]c12)c1cccc(S(=O)(=O)N2CCNCC2)c1]

### 1. Preparation of 1-((3-bromophenyl)sulfonyl)piperazine

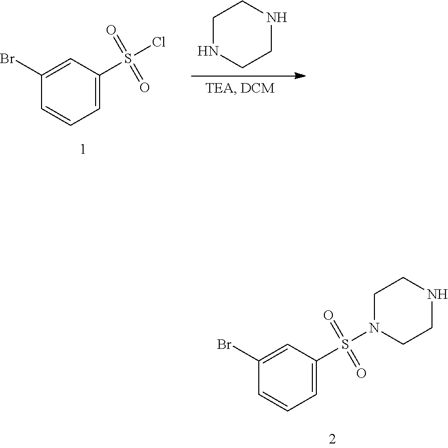

To a solution of 3-bromobenzenesulfonyl chloride (200 mg, 0.78 mmol) in DCM (5 mL) was added TEA (335.4 mg, 3.90 mmol). The reaction mixture was stirred for 1 h at rt. The reaction mixture was diluted with water (30 mL) and extracted with DCM (50 mL×2). The organic layer was washed with water (20 mL), brine (20 mL), dried over Na₂SO₄ and concentrated under reduced pressure to afford the crude product. The crude product was purified by column chromatography on silica gel eluted with PE/EA from 10/1 to 2/1 to give 1-((3-bromophenyl)sulfonyl)piperazine (180.0 mg, 95.4%) as a white solid. LCMS (ESI) found: 305 [M+H]⁺.

### 2. Preparation of 1-(4-((3-bromophenyl)sulfonyl)piperazin-1-yl)-2,2,2-trifluoroethan-1-one

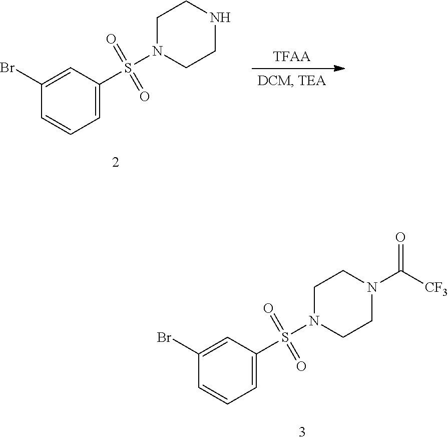 [SMILES: O=S(=O)(c1cccc(Br)c1)N1CCNCC1 ; "TFAA" ; "DCM, TEA" ; O=C(N1CCN(S(=O)(=O)c2cccc(Br)c2)CC1)C(F)(F)F]

To a solution of 1-((3-bromophenyl)sulfonyl)piperazine (180.0 mg, 0.75 mmol) in DCM (5 mL) were added TEA (189.4 mg, 1.87 mmol) and TFAA (236.2 mg, 1.12 mmol). The reaction mixture was stirred for 1 h at rt. The reaction mixture was diluted with water (25 mL) and extracted with DCM (25 mL×3). The organic layer was washed with water (20 mL), brine (20 mL), dried over Na₂SO₄ and concentrated under reduced pressure to afford the crude product, which was purified by column chromatography on silica gel eluted with DCM/MEOH (pure DCM to 10/1) to give 1-(4-((3-bromophenyl)sulfonyl)piperazin-1-yl)-2,2,2-trifluoroethan-1-one (193.1 mg, 64.4%) as a white solid. LCMS (ESI) found: 401 [M+H]⁺.

### 3. Preparation of 1-(4-((3-(benzylthio)phenyl)sulfonyl)piperazin-1-yl)-2,2,2-trifluoroethan-1-one

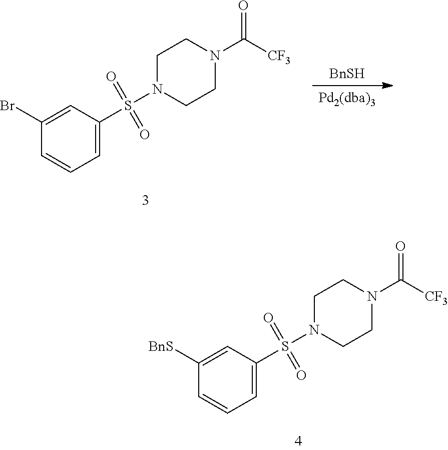

To a mixture of 1-(4-((3-bromophenyl)sulfonyl)piperazin-1-yl)-2,2,2-trifluoroethan-1-one (100.0 mg, 0.30 mmol) and DIEA (96.7 mg, 0.75 mmol) in dioxane (3 mL) were added BnSH (44.6 mg, 0.36 mmol), Pd₂(dba)₃ (27.4 mg, 0.03 mmol) and Xantphos (34.7 mg, 0.06 mmol). The mixture was evaporated and backfilled with N₂ for three times. The mixture was stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give 1-(4-((3-(benzylthio)phenyl)sulfonyl)piperazin-1-yl)-2,2,2-trifluoroethan-1-one (90.0 mg, 79.8%) as a white solid. LCMS (ESI) found: 445 [M+H]⁺.

### 4. Preparation of 1-(4-((3-(benzylthio)phenyl)sulfonyl)piperazin-1-yl)-2,2,2-trifluoroethan-1-one

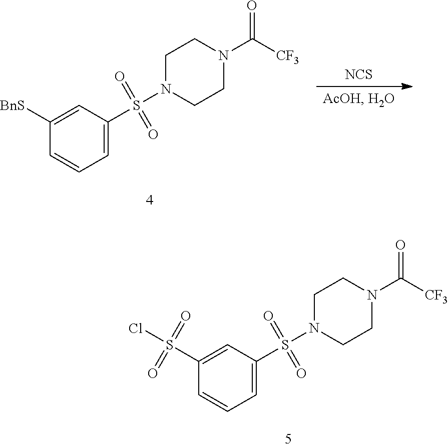

To a solution of 1-(4-((3-(benzylthio)phenyl)sulfonyl)piperazin-1-yl)-2,2,2-trifluoroethan-1-one (90.0 mg, 0.24 mmol) in AcOH/H₂O (9/1, 2 mL) was added NCS (135.1 mg, 1.01 mmol). The mixture was stirred at rt for 2 h. The mixture was concentrated and purified by prep-TLC eluted with PE/EA/DCM=3/1/1 to give 3-((4-(2,2,2-trifluoroacetyl)piperazin-1-yl)sulfonyl)benzenesulfonyl chloride (62.0 mg, 61.4%) as a white solid. LCMS (ESI) found: 421 [M+H]⁺.

### 5. Preparation of N-(3,4-dichloro-1H-indol-7-yl)-3-((4-(2,2,2-trifluoroacetyl)piperazin-1-yl)sulfonyl)benzenesulfonamide

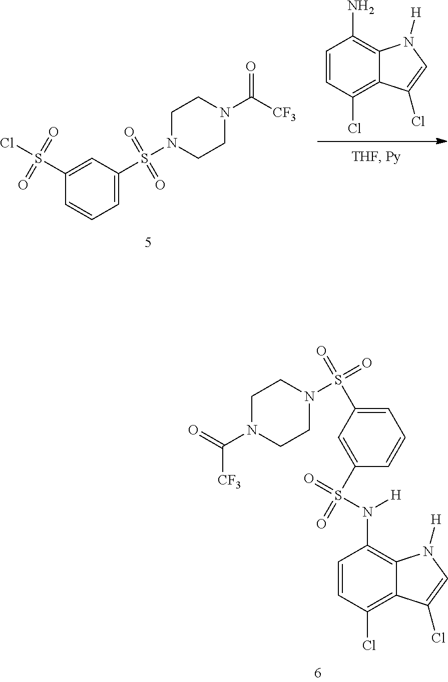 [SMILES: O=C(N1CCN(S(=O)(=O)c2cccc(S(=O)(=O)Cl)c2)CC1)C(F)(F)F ; Nc1ccc(Cl)c2c(Cl)c[nH]c12 ; O=C(N1CCN(S(=O)(=O)c2cccc(S(=O)(=O)Nc3ccc(Cl)c4c(Cl)c[nH]c34)c2)CC1)C(F)(F)F]

To a solution of 3,4-dichloro-1H-indol-7-amine (29.6 mg, 0.15 mmol) and pyridine (34.9 mg, 0.44 mmol) in THF (2 mL) was added 3-((4-(2,2,2-trifluoroacetyl)piperazin-1-yl)sulfonyl)benzenesulfonyl chloride (62 mg, 0.15 mmol). The mixture was stirred at rt for 4 h. The mixture was concentrated and purified by prep-TLC eluted with DCM/MeOH=15/1 to give N-(3,4-dichloro-1H-indol-7-yl)-3-((4-(2,2,2-trifluoroacetyl)piperazin-1-yl)sulfonyl)benzenesulfonamide (40.0 mg, 46.4%) as a white solid. LCMS (ESI) found: 585 [M+H]⁺.

### 6. Preparation of N-(3,4-dichloro-1H-indol-7-yl)-3-(piperazin-1-ylsulfonyl)benzenesulfonamide

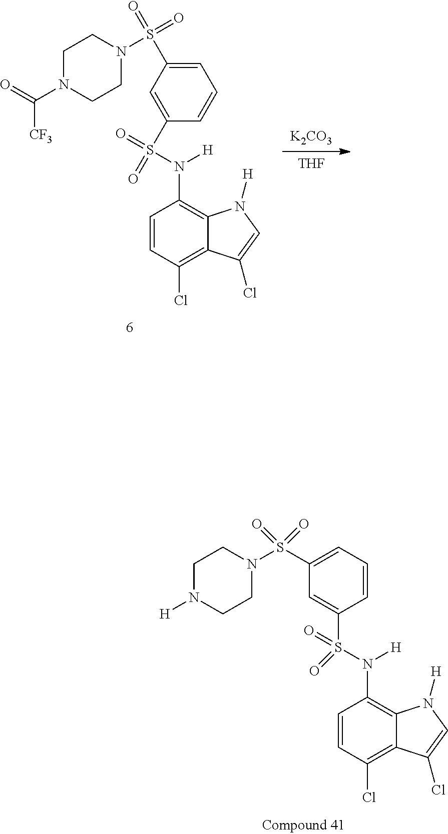 [SMILES: O=C(N1CCN(S(=O)(=O)c2cccc(S(=O)(=O)Nc3ccc(Cl)c4c(Cl)c[nH]c34)c2)CC1)C(F)(F)F ; "K2CO3" ; "THF" ; O=S(=O)(Nc1ccc(Cl)c2c(Cl)c[nH]c12)c1cccc(S(=O)(=O)N2CCNCC2)c1]

To a solution of N-(3,4-dichloro-1H-indol-7-yl)-3-((4-(2,2,2-trifluoroacetyl)piperazin-1-yl)sulfonyl)benzenesulfonamide (40.0 mg, 0.07 mmol) in THF/H₂O (511, 2 mL) was added K₂CO₃ (28.3 mg, 0.21 mmol). The mixture was stirred at 50° C. overnight. The mixture was cooled to rt, filtered and concentrated to give a residue, which was purified by prep-TLC eluted with DCM/MeOH=15/1 to give N-(3,4-dichloro-1H-indol-7-yl)-3-(piperazin-1-ylsulfonyl)benzenesulfonamide (Compound 41, 29.5 mg, 88.2%) as a white solid. LCMS (ESI) found: 489 [M+H]⁺. ¹H NMR (400 MHz, DMSO) δ 11.22 (s, 1H), 8.05-7.90 (m, 2H), 7.66-7.46 (m, 2H), 7.15 (s, 1H), 6.74-6.66 (m, 1H), 6.65-6.57 (m, 1H), 2.62 (s, 4H), 2.51 (s, 4H).

### Example 12: Preparation of N-(3,4-dichloro-1H-indol-7-yl)-4-(piperazin-1-ylsulfonyl)benzenesulfonamide; Compound 42

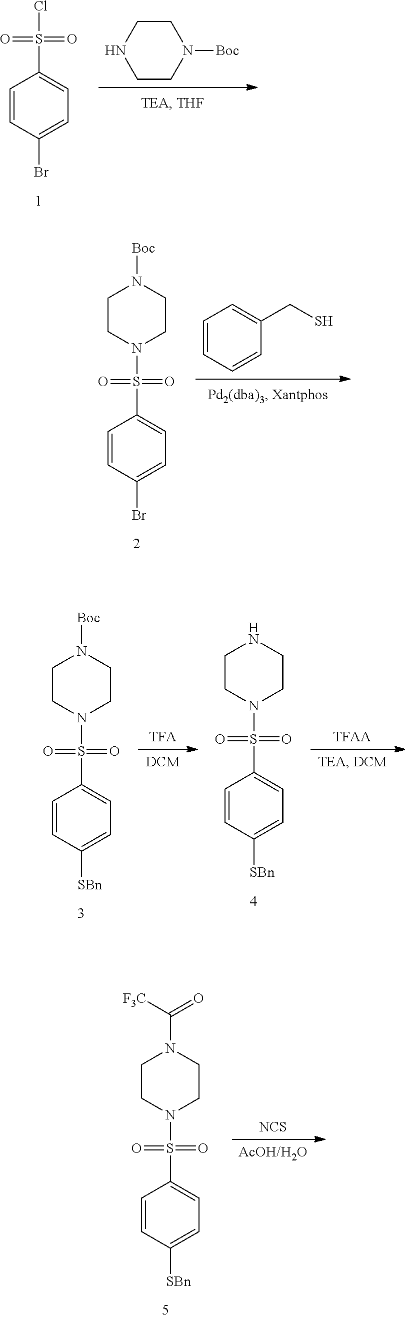

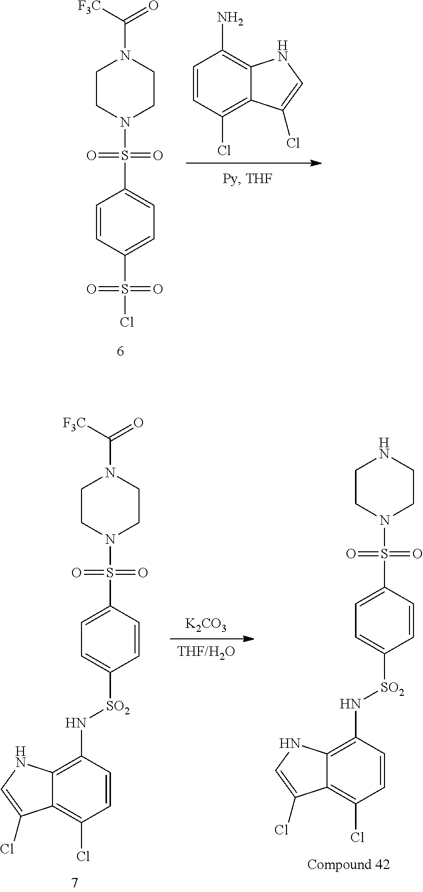

### 1. Preparation of tert-butyl 4-((4-bromophenyl)sulfonyl)piperazine-1-carboxylate

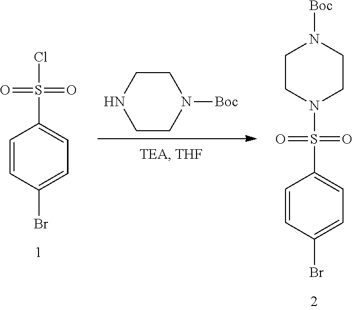 [SMILES: O=S(=O)(Cl)c1ccc(Br)cc1 ; CC(C)(C)OC(=O)N1CCNCC1 ; CC(C)(C)OC(=O)N1CCN(S(=O)(=O)c2ccc(Br)cc2)CC1]

To a solution of tert-butyl piperazine-1-carboxylate (232.5 mg, 1.25 mmol) and TEA (378.7 mg, 3.75 mmol) in THF (10 mL) was added 4-bromobenzenesulfonyl chloride (318.8 mg, 1.25 mmol). The mixture was stirred at rt for 3 h until the starting material was consumed completely. The reaction mixture was concentrated in vacuum to remove most of solvent. The residue was poured into water (20 mL) and extracted with EA (20 mL×3). The combined organic layer was dried over MgSO₄, filtered and concentrated to afford a crude product, which was purified by column chromatography on silica gel eluting with PE/EA=10/1 to 5/1 to give tert-butyl 4-((4-bromophenyl)sulfonyl)piperazine-1-carboxylate (316.2 mg, yield 62.4%) as a white solid. LCMS (ESI) found: 405 [M+H]⁺.

### 2. Preparation of tert-butyl 4-((4-(benzylthio)phenyl)sulfonyl)piperazine-1-carboxylate

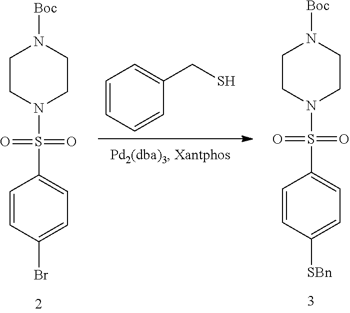

To a solution of tert-butyl 4-((4-bromophenyl)sulfonyl)piperazine-1-carboxylate (187.2 mg, 0.46 mmol) and BnSH (57.5 mg, 0.46 mmol) in dioxane (2 mL) were added DIEA (148.3 mg, 1.15 mmol), Pd₂(dba)₃ (45.7 mg, 0.05 mmol) and Xantphos (57.8 mg, 0.10 mmol). The mixture was charged with N₂ for three times. The mixture was stirred at 90° C. for 3 hours under N₂ atmosphere until the starting material was consumed completely. The reaction mixture was cooled to room temperature, filtered and concentrated in vacuum to remove most of solvent. The residue was poured into water (20 mL) and extracted with EA (20 mL×3). The combined organic layer was dried over MgSO₄, filtered and concentrated to afford a crude product, which was purified by column chromatography on silica gel eluting with PE/EA from 40/1 to 25/1 to give tert-butyl 4-((4-(benzylthio)phenyl)sulfonyl) piperazine-1-carboxy late (154.3 mg, yield: 74.3%) as a yellow solid. LCMS (ESI) found: 449 [M+H]⁺.

### 3. Preparation of 1-((4-(benzylthio)phenyl)sulfonyl)piperazine

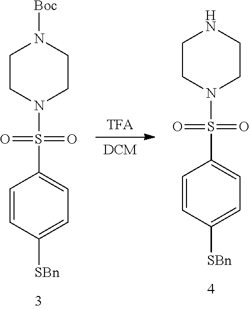

To a solution of tert-butyl 4-((4-(benzylthio)phenyl)sulfonyl)piperazine-1-carboxylate (100.0 mg, 0.22 mmol) in DCM (2 mL) was added TFA (0.4 mL). The mixture was stirred at rt for 2 h. The mixture was concentrated and diluted with DCM (20 mL). The mixture was washed with brine (20 mL) and aqueous NaHCO₃ (20 mL). The organic layer was concentrated to give crude 1-((4-(benzylthio) phenyl)sulfonyl)piperazine (108.0 mg, crude) as a white solid, which was used for next step directly. LCMS (ESI) found: 349 [M+H]⁺.

### 4. Preparation of 1-(4-((4-(benzylthio)phenyl)sulfonyl)piperazin-1-yl)-2,2,2-trifluoro ethan-1-one

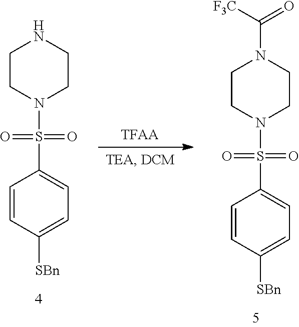

To a solution of crude 1-((4-(benzylthio)phenyl)sulfonyl)piperazine (108.0 mg, 0.22 mmol) in DCM (2 mL) were added TEA (66.7 mg, 0.66 mmol) and TFAA (69.3 mg, 0.33 mmol). The mixture was stirred at rt for 4 h. The reaction mixture was diluted with water (15 mL) and extracted with DCM (15 mL×3). The combined organic layer was washed with water (10 mL), brine (10 mL), dried over Na₂SO₄ and concentrated under reduced pressure to afford the crude product, which was purified by column chromatography on silica gel eluted with DCM/MeOH (pure DCM to DCM/MeOH=10/1) to give 1-(4-((4-(benzylthio)phenyl)sulfonyl)piperazin-1-yl)-2,2,2-trifluoroethan-1-one (81.2 mg, 83.1%) as a yellow solid. LCMS (ESI) found: 445 [M+H]⁺.

### 5. Preparation of 4-((4-(2,2,2-trifluoroacetyl)piperazin-1-yl)sulfonyl)benzenesulfonyl chloride

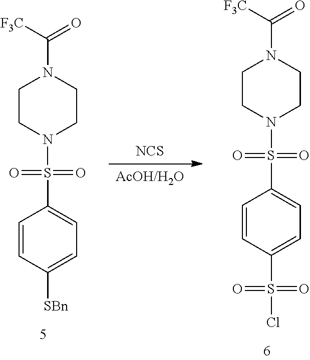

To a solution of 1-(4-((4-(benzylthio)phenyl)sulfonyl)piperazin-1-yl)-2,2,2-trifluoroethan-1-one (50.0 mg, 0.11 mmol) in AcOH/H₂O (9/1, 2 mL) was added NCS (60.1 mg, 0.45 mmol). The mixture was stirred at rt for 2 h. The reaction was quenched with aqueous Na₂S₂O₃ (10 mL). The mixture was extracted with DCM (15 mL×3). The combined organic layer was concentrated to afford a crude product, which was purified by column chromatography on silica gel eluting with PE/EA from 15/1 to 5/1 to afford 4-((4-(2,2,2-trifluoroacetyl)piperazin-1-yl)sulfonyl)benzenesulfonyl chloride (38.3 mg, 81.8%) as a white solid. LCMS (ESI) found: 421 [M+H]⁺.

### 6. Preparation of trifluoromethyl 4-((4-(N-(3,4-dichloro-1H-indol-7-yl)sulfamoyl)phenyl) sulfonyl)piperazine-1-carboxylate

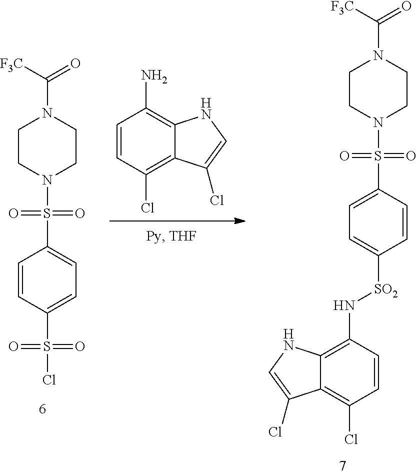

To a solution of 3,4-dichloro-1H-indol-7-amine (30.6 mg, 0.15 mmol) and Py. (36.1 mg, 0.45 mmol) in THF (2 mL) was added 4-((4-(2,2,2-trifluoroacetyl)piperazin-1-yl)sulfonyl) benzenesulfonyl chloride (63.1 mg, 0.15 mmol). The mixture was stirred at rt for 3 h. The mixture was concentrated to afford a crude product, which was purified by column chromatography on silica gel eluting with PE/EA from 15/1 to 5/1 to afford N-(3,4-dichloro-1H-indol-7-yl)-4-((4-(2,2,2-trifluoroacetyl)piperazin-1-yl)sulfonyl) benzene sulfonamide (57.0 mg, 66.7%) as a white solid. LCMS (ESI) found: 585 [M+H]⁺.

### 7. Preparation of N-(3,4-dichloro-1H-indol-7-yl)-4-(piperazin-1-ylsulfonyl)benzenesulfonamide

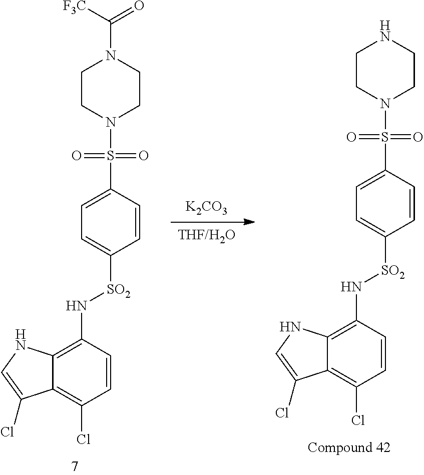

To a solution of N-(3,4-dichloro-1H-indol-7-yl)-4-((4-(2,2,2-trifluoroacetyl)piperazin-1-yl) sulfonyl)benzenesulfonamide (45.0 mg, 0.08 mmol) in THF/H₂O (4/1, 2 mL) was added K₂CO₃ (212.4 mg, 1.54 mmol). The mixture was stirred at 50° C. for 2 h. The mixture was cooled to room temperature and filtered. The filtrated was diluted with water (10 mL) and extracted with EA (15 mL×3). The combined organic layer was concentrated to give a crude product, which was purified by column chromatography on silica gel eluting with PE/EA from 3/1 to 1/1 to afford N-(3,4-dichloro-1H-indol-7-yl)-4-(piperazin-1-ylsulfonyl)benzenesulfonamide (Compound 42, 8.9 mg, 22.8%) as a white solid. LCMS (ESI) found: 489 [M+H]⁺. ¹H NMR (400 MHz, DMSO-d6) δ 11.36 (s, 1H), 7.95 (d, J=8.5 Hz, 2H), 7.82 (d, J=8.5 Hz, 2H), 7.39 (s, 1H), 6.82 (d, J=8.2 Hz, 1H), 6.64 (d, J=8.2 Hz, 1H), 2.90 (s, 8H).

### Example 13: Preparation of 3-cyano-N-(3,4-dichloro-1H-indol-7-yl)-4-(piperazin-1-ylsulfonyl)benzene sulfonamide; Compound 43

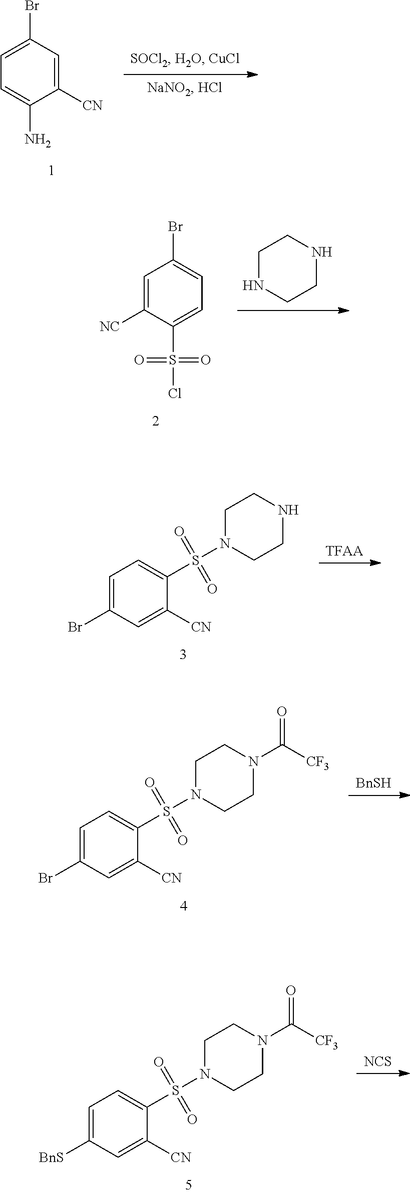

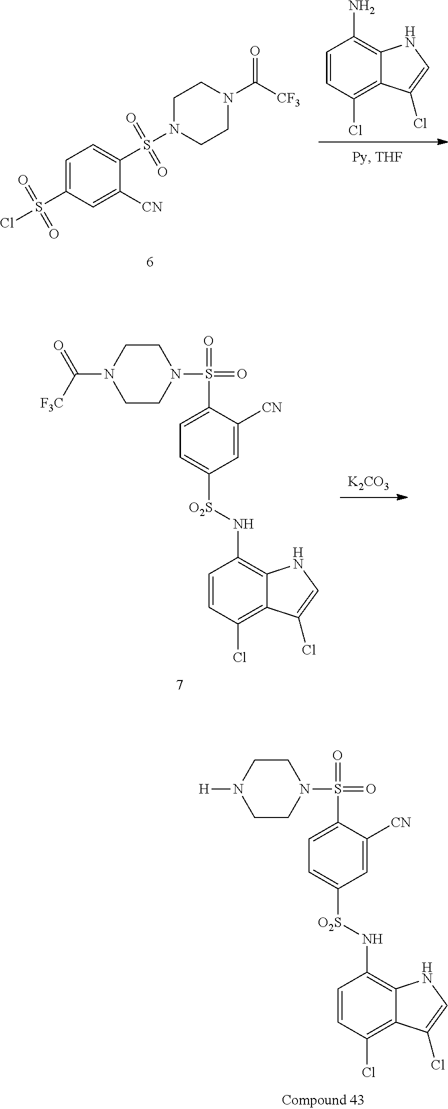 [SMILES: N#Cc1cc(S(=O)(=O)Cl)ccc1S(=O)(=O)N1CCN(C(=O)C(F)(F)F)CC1 ; Nc1ccc(Cl)c2c(Cl)c[nH]c12 ; N#Cc1cc(S(=O)(=O)Nc2ccc(Cl)c3c(Cl)c[nH]c23)ccc1S(=O)(=O)N1CCN(C(=O)C(F)(F)F)CC1 ; "K2CO3" ; N#Cc1cc(S(=O)(=O)Nc2ccc(Cl)c3c(Cl)c[nH]c23)ccc1S(=O)(=O)N1CCNCC1]

### Preparation of 4-bromo-2-cyanobenzenesulfonyl chloride

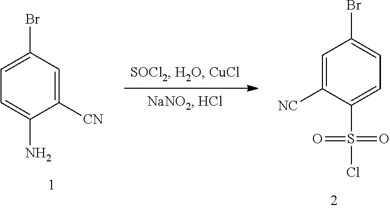

To a round bottom flask containing iced water (40.0 mL, 2.22 mmol) was carefully added SOCl₂ (12.3 g, 104.24 mmol) at 0° C. The mixture was stirred at rt for 2 h. Then CuCl (0.24 g, 2.54 mmol) was added and the mixture was stirred at rt for 10 mins to give a solution A. To another flask containing 2-amino-5-bromobenzonitrile (5.0 g, 25.51 mmol) were carefully added NaNO₂ (2.0 g, 27.92 mmol) and HCl (13.6 mL, 263.14 mmol) at 0° C. The mixture was stirred at rt for 15 mins to give a solution B. The solution B was dropped to solution A slowly and the mixture was stirred at rt for 2 h. The reaction mixture was poured into water (100 mL) and extracted with EA (200 mL×3). The combined organic layer was dried over MgSO₄, filtered and concentrated to give a crude product, which was purified by column chromatography on silica gel eluting with PE/EA from 5/1 to 3/1 to give 4-bromo-2-cyanobenzenesulfonyl chloride (5.0 g, yield: 70.0%) as a yellow solid. LCMS (ESI) found: 280 [M+H]⁺.

### 2. Preparation of 5-bromo-2-(piperazin-1-ylsulfonyl)benzonitrile

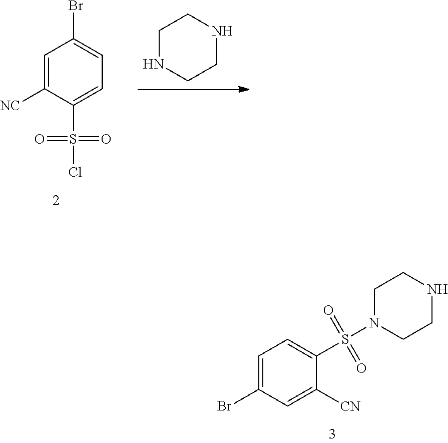 [SMILES: N#Cc1cc(Br)ccc1S(=O)(=O)Cl ; C1CNCCN1 ; N#Cc1cc(Br)ccc1S(=O)(=O)N1CCNCC1]

To a solution of 4-bromo-2-cyanobenzenesulfonyl chloride (5.0 g, 17.86 mmol) in DCM (100 mL) were added TEA (4.4 g, 43.88 mmol) and piperazine (8.7 g, 100.7 mmol). The mixture was stirred at rt for 3 h until the starting material was consumed completely. The mixture was poured into water (200 mL) and extracted with DCM (250 mL×3). The combined organic layer was dried over MgSO₄, filtered and concentrated to give a crude product, which was purified by flash eluting with DCM/MeOH from 25/1 to 20/1 to give 5-bromo-2-(piperazin-1-ylsulfonyl)benzonitrile (4.5 g, yield: 76.3%) a white solid. LCMS (ESI) found: 330 [M+H]⁺.

### 3. Preparation of 5-bromo-2-((4-(2,2,2-trifluoroacetyl)piperazin-1-yl)sulfonyl)benzonitrile

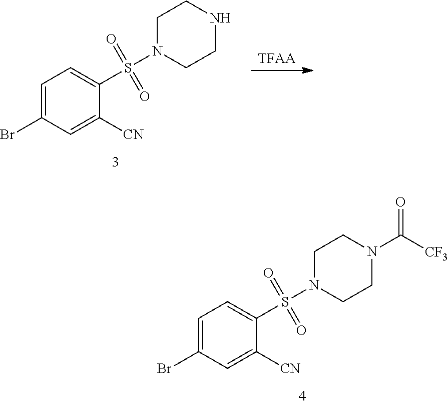 [SMILES: N#Cc1cc(Br)ccc1S(=O)(=O)N1CCNCC1 ; "TFAA" ; N#Cc1cc(Br)ccc1S(=O)(=O)N1CCN(C(=O)C(F)(F)F)CC1]

To a solution of 5-bromo-2-(piperazin-1-ylsulfonyl)benzonitrile (4.0 g, 12.12 mmol) in DCM (50 mL) were added TEA (2.5 g, 24.76 mmol) and TFAA (2.8 g, 13.62 mmol). The mixture was stirred at rt for 1 h until the starting material was consumed completely. The reaction mixture was diluted with DCM (50 mL), washed with water (50 mL×2). The organic layer was concentrated to give a crude product, which was purified by flash column to give 5-bromo-2-((4-(2,2,2-trifluoroacetyl) piperazin-1-yl)sulfonyl)benzonitrile (4.5 g, yield: 87.2%) as a yellow solid. LCMS (ESI) found: 426 [M+H]⁺.

### 4. Preparation of 5-(benzylthio)-2-((4-(2,2,2-trifluoroacetyl)piperazin-1-yl)sulfonyl) benzonitrile

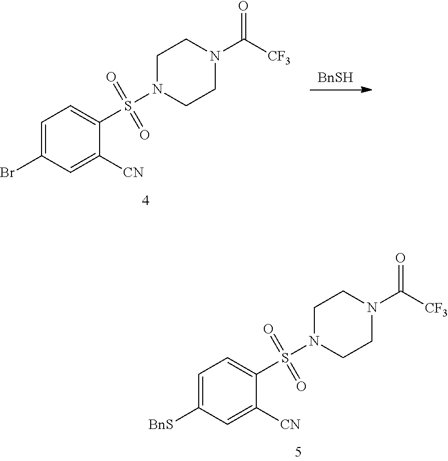

To a solution of 5-bromo-2-((4-(2,2,2-trifluoroacetyl) piperazin-1-yl)sulfonyl)benzonitrile (1.0 g, 2.35 mmol) and BnSH (296.3 mg, 2.39 mmol) in Dioxane (5 mL) were added DIEA (770.8 mg, 5.97 mmol), Pd₂(dba)₃ (219.8 mg, 0.24 mmol) and Xantphos (277.4 mg, 0.48 mmol). The mixture was charged with N₂ for three times and stirred at 110° C. for 2 h under N₂ atmosphere until the starting material was consumed completely. The reaction mixture was cooled to room temperature and filtered. The filtrated was concentrated to give a crude product, which was purified by flash column eluting with PE/EA from 5/1 to 3/1 to give 5-(benzylthio)-2-((4-(2,2,2-trifluoroacetyl)piperazin-1-yl)sulfonyl) benzonitrile (900.0 mg, yield: 81.5%) as a yellow solid. LCMS (ESI) found: 470 [M+H]⁺.

### 5. Preparation of 3-cyano-4-((4-(2,2,2-trifluoroacetyl)piperazin-1-yl)sulfonyl)benzene sulfonyl chloride

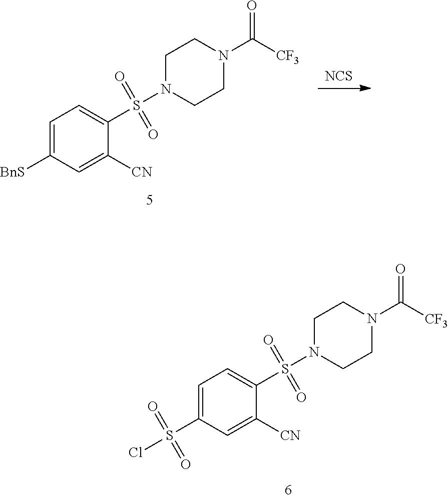

To a solution of 5-(benzylthio)-2-((4-(2,2,2-trifluoroacetyl)piperazin-1-yl)sulfonyl)benzonitrile (400.0 mg, 0.85 mmol) in AcOH/H₂O (9/1, 4.5 mL) was added NCS (487.6 mg, 3.61 mmol). The mixture was stirred at rt for 1 hour until the starting material was consumed completely. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 3-cyano-4-((4-(2,2,2-trifluoroacetyl)piperazin-1-yl)sulfonyl)benzenesulfonyl chloride (260.0 mg, yield: 68.6%) as a white solid. LCMS (ESI) found: 446 [M+H]⁺.

### 6. Preparation of 3-cyano-N-(3,4-dichloro-1H-indol-7-yl)-4-((4-(2,2,2-trifluoroacetyl) piperazin-1-yl)sulfonyl)benzenesulfonamide

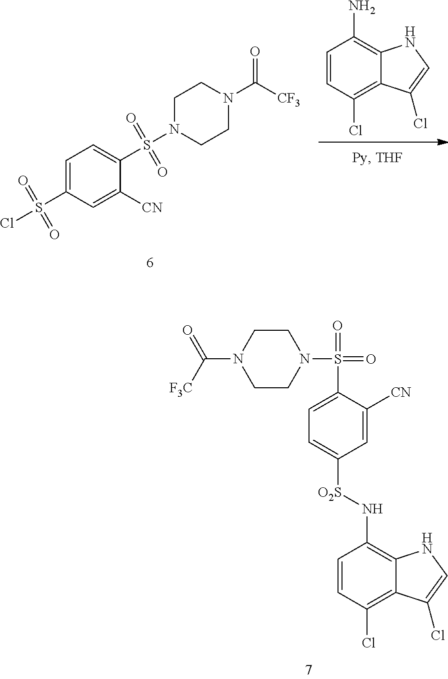 [SMILES: N#Cc1cc(S(=O)(=O)Cl)ccc1S(=O)(=O)N1CCN(C(=O)C(F)(F)F)CC1 ; Nc1ccc(Cl)c2c(Cl)c[nH]c12 ; N#Cc1cc(S(=O)(=O)Nc2ccc(Cl)c3c(Cl)c[nH]c23)ccc1S(=O)(=O)N1CCN(C(=O)C(F)(F)F)CC1]

To a solution of 3,4-dichloro-1H-indol-7-amine (40.0 mg, 0.20 mmol) and Pyridine (54.5 mg, 0.69 mmol) in THF (3 mL) was added 2-fluoro-4-((4-(2,2,2-trifluoroacetyl)piperazin-1-yl) sulfonyl)benzene sulfonyl chloride (100.0 mg, 0.22 mmol). The mixture was stirred at rt for 18 h until the starting material was consumed completely. The reaction mixture was concentrated to give a crude product, which was purified by prep-HPLC to give 3-cyano-N-(3,4-dichloro-1H-indol-7-yl)-4-((4-(2,2,2-trifluoroacetyl)piperazin-1-yl)sulfonyl)benzenesulfonamide (80.0 mg, yield: 65.6%) as white solid. LCMS (ESI) found: 610 [M+H]⁺.

### 7. Preparation of 3-cyano-N-(3,4-dichloro-1H-indol-7-yl)-4-(piperazin-1-ylsulfonyl)benzene sulfonamide

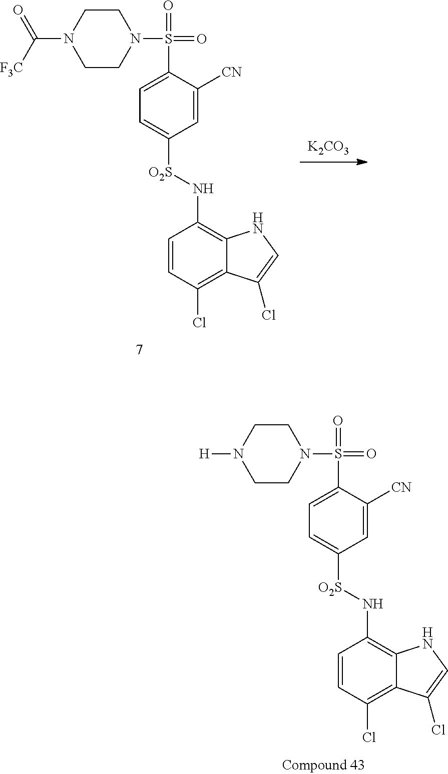 [SMILES: N#Cc1cc(S(=O)(=O)Nc2ccc(Cl)c3c(Cl)c[nH]c23)ccc1S(=O)(=O)N1CCN(C(=O)C(F)(F)F)CC1 ; "K2CO3" ; N#Cc1cc(S(=O)(=O)Nc2ccc(Cl)c3c(Cl)c[nH]c23)ccc1S(=O)(=O)N1CCNCC1]

To a solution of 3-cyano-N-(3,4-dichloro-1H-indol-7-yl)-4-((4-(2,2,2-trifluoroacetyl) piperazin-1-yl)sulfonyl)benzenesulfonamide (80.0 mg, 0.13 mmol) in THF/H₂O (4/1, 10 mL) was added K₂CO₃ (53.8 mg, 0.39 mmol). The mixture was stirred at 70° C. for 4 h until the starting material was consumed completely. The mixture was filtered and concentrated to give a crude product, which was purified by prep-HPLC to give 3-cyano-N-(3,4-dichloro-1H-indol-7-yl)-4-(piperazin-1-ylsulfonyl)benzenesulfonamide (HCOOH salt) (Compound 43, 55.3 mg, yield: 82.8%) as a white solid. LCMS (ESI) found: 514 [M+H]⁺. ¹H NMR (400 MHz, DMSO-d6) δ 11.33 (s, 1H), 8.34 (s, 1H), 8.15 (d, J=8.0 Hz, 1H), 8.14 (s, 0.85H), 8.05 (d, J=8.3 Hz, 1H), 7.31 (s, 1H), 6.79-6.75 (m, 2H), 3.21-3.17 (m, 4H), 3.08-3.04 (m, 4H).

### Example 14: Preparation of N-(3,4-dichloro-1H-indol-7-yl)-2-fluoro-4-(piperazin-1-ylsulfonyl)benzenesulfonamide: Compound 44

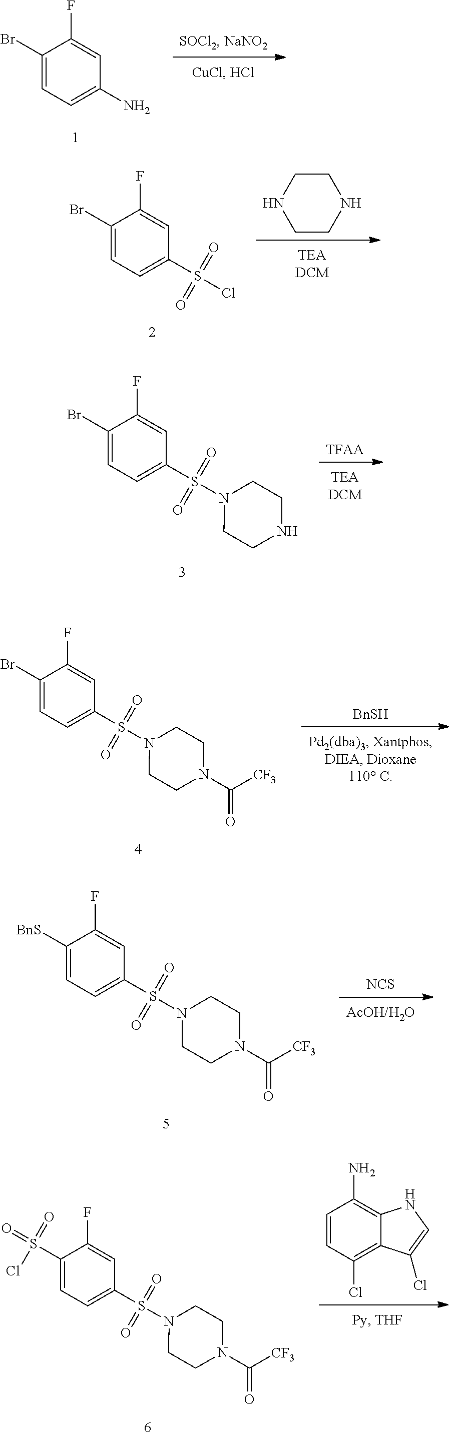

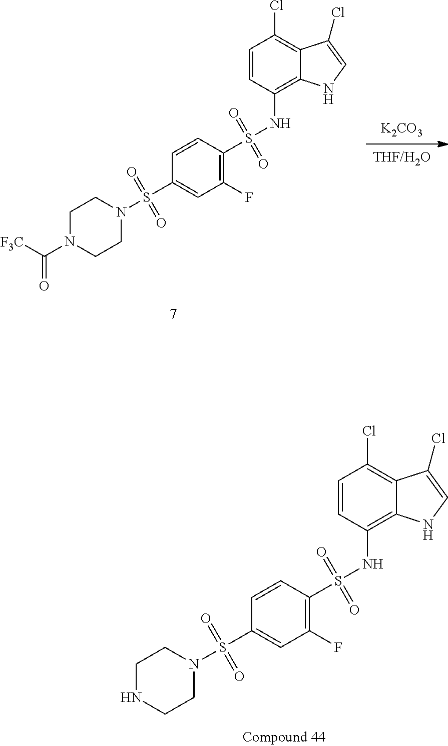

### 1. Preparation of 4-bromo-3-fluorobenzenesulfonyl chloride

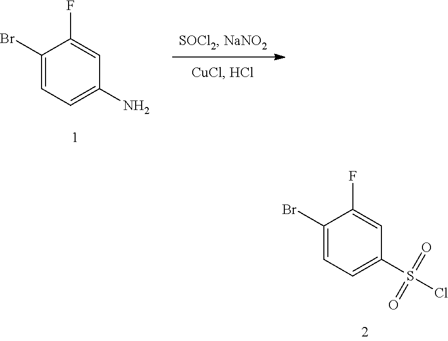 [SMILES: Nc1ccc(Br)c(F)c1 ; O=S(=O)(Cl)c1ccc(Br)c(F)c1]

To a round bottom flask containing ice water (40.0 mL, 2.22 mmol) was carefully added SOCl₂ (12.3 g, 104.24 mmol) at 0° C. The mixture was stirred at rt for 2 h. Then CuCl (0.24 g, 2.54 mmol) was added and the mixture was stirred at rt for 10 mins to give a solution A. To another round bottom flask containing 4-bromo-3-fluoroaniline (5.0 g, 26.31 mmol) were carefully added NaNO₂ (2.0 g, 27.92 mmol) and HCl (13.6 mL, 263.14 mmol) at 0° C. The mixture was stirred at rt for 15 mins to give a solution B. The solution B was added dropwise to solution A. The mixture was stirred at rt for 2 h. Then the reaction mixture was poured into water (100 mL) and extracted with EA (200 mL×3). The combined organic layer was dried over MgSO₄, filtered and concentrated to give a crude product, which was purified by column chromatography on silica gel eluting with PE/EA from 5/1 to 3/1 to give 4-bromo-3-fluorobenzenesulfonyl chloride (5.5 g, yield: 76.6%) as a green oil. LCMS (ESI) found: 273 [M+H]⁺.

### 2. Preparation of 1-((4-bromo-3-fluorophenyl)sulfonyl)piperazine

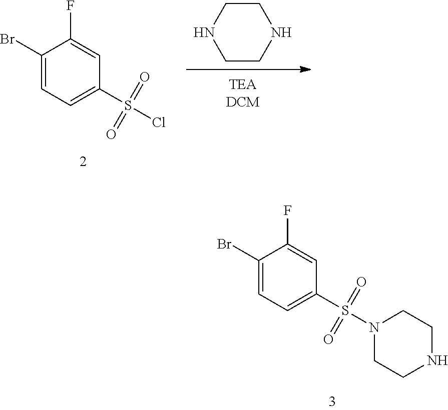 [SMILES: O=S(=O)(Cl)c1ccc(Br)c(F)c1 ; C1CNCCN1 ; O=S(=O)(c1ccc(Br)c(F)c1)N1CCNCC1]

To a solution of 4-bromo-3-fluorobenzene-1-sulfonyl chloride (5.0 g, 18.25 mmol) in DCM (100 mL) were added TEA (4.4 g, 43.88 mmol) and piperazine (8.7 g, 100.7 mmol). The mixture was stirred at rt for 3 h until the starting material was consumed completely. The mixture was poured into water (100 mL) and extracted with DCM (150 mL×3). The combined organic layer was dried over MgSO₄, filtered and concentrated to give a crude product, which was purified by flash eluting with DCM/MeOH from 25/1 to 20/1 to give 1-((4-bromo-3-fluorophenyl)sulfonyl)piperazine (4.5 g, yield: 76.3%) a white solid. LCMS (ESI) found: 323 [M+H]⁺.

### 3. Preparation of 1-(4-((4-bromo-3-fluorophenyl)sulfonyl)piperazin-1-yl)-2,2,2-trifluoro ethan-1-one

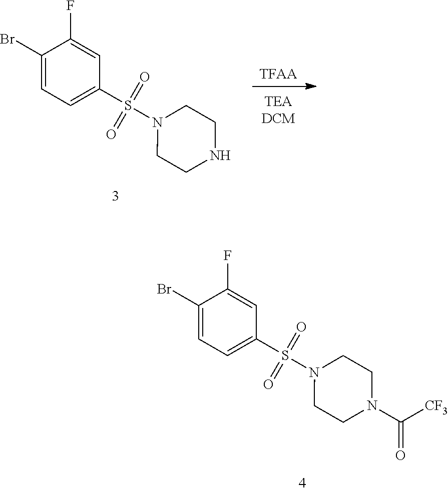 [SMILES: O=S(=O)(c1ccc(Br)c(F)c1)N1CCNCC1 ; "TFAA" ; O=C(N1CCN(S(=O)(=O)c2ccc(Br)c(F)c2)CC1)C(F)(F)F]

To a solution of 1-(4-bromo-3-fluorobenzenesulfonyl)piperazine (4.0 g, 12.38 mmol) in DCM (50 mL) were added TEA (2.5 g, 24.76 mmol) and TFAA (2.8 g, 13.62 mmol). The mixture was stirred at rt for 1 hours until the starting material was consumed completely. The reaction mixture was diluted with DCM (150 mL) and washed with water (100 mL×2). The organic layer was separated and concentrated to give a crude product, which was purified by flash column to give 1-(4-((4-bromo-3-fluorophenyl)sulfonyl)piperazin-1-yl)-2,2,2-trifluoro ethan-1-one (4.5 g, yield: 86.7%) as a yellow solid. LCMS (ESI) found: 419 [M+H]⁺.

### 4. Preparation of 1-(4-((4-(benzylthio)-3-fluorophenyl)sulfonyl)piperazin-1-yl)-2,2,2-trifluoroethan-1-one

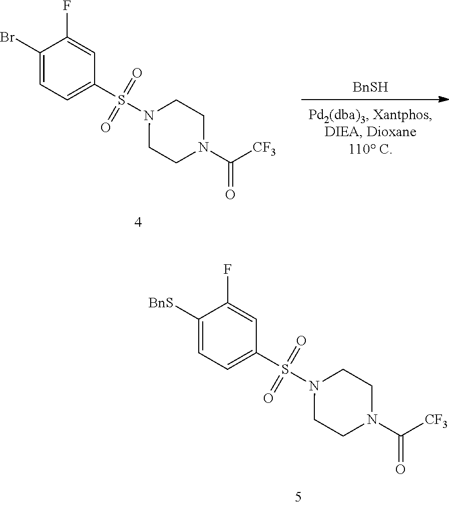

To a solution of 1-(4-((4-bromo-3-fluorophenyl)sulfonyl)piperazin-1-yl)-2,2,2-trifluoro ethan-1-one (1.0 g, 2.39 mmol) and BnSH (296.3 mg, 2.39 mmol) in Dioxane (5 mL) were added DIEA (770.8 mg, 5.97 mmol), Pd₂(dba)₃ (219.8 mg, 0.24 mmol) and Xantphos (277.4 mg, 0.48 mmol). The mixture was charged with N₂ for three times and stirred at 110° C. under N₂ atmosphere for 3 h until the starting material was consumed completely. The mixture was cooled to room temperature and concentrated in vacuum to remove most of solvent. The residue was diluted with water (50 mL) and extracted with EA (50 mL×3). The combined organic layer was dried over MgSO₄, filtered and concentrated to give a crude product, which was purified by column chromatography on silica gel eluting with PE/EA from 30/1 to 5/1 to give 1-(4-((4-(benzylthio)-3-fluorophenyl)sulfonyl)piperazin-1-yl)-2,2,2-trifluoroethan-1-one (900.0 mg, yield: 81.3%) as a yellow solid. LCMS (ESI) found: 463 [M+H]⁺.

### 5. Preparation of 2-fluoro-4-((4-(2,2,2-trifluoroacetyl)piperazin-1-yl)sulfonyl)benzene sulfonyl chloride

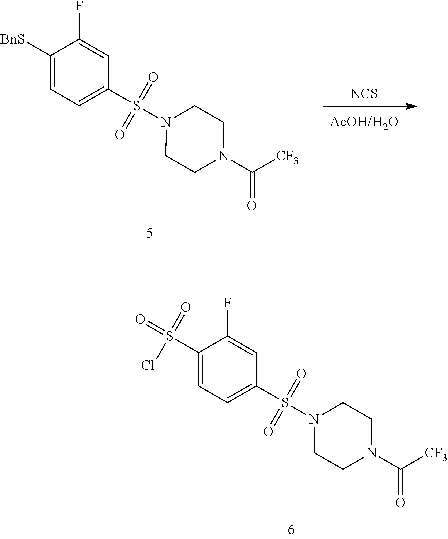

To a solution of 1-(4-((4-(benzylthio)-3-fluorophenyl)sulfonyl)piperazin-1-yl)-2,2,2-trifluoroethan-1-one (400.0 mg, 0.86 mmol) in AcOH/H₂O (9/1, 4.5 mL) was added NCS (487.6 mg, 3.61 mmol). The mixture was stirred at rt for 2 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 2-fluoro-4-((4-(2,2,2-trifluoroacetyl)piperazin-1-yl)sulfonyl)benzene sulfonyl chloride (200.0 mg, yield: 52.3%) as a white solid. LCMS (ESI) found: 439 [M+H]⁺.

### 6. Preparation of N-(3,4-dichloro-1H-indol-7-yl)-2-fluoro-4-((4-(2,2,2-trifluoroacetyl) piperazin-1-yl)sulfonyl)benzenesulfonamide

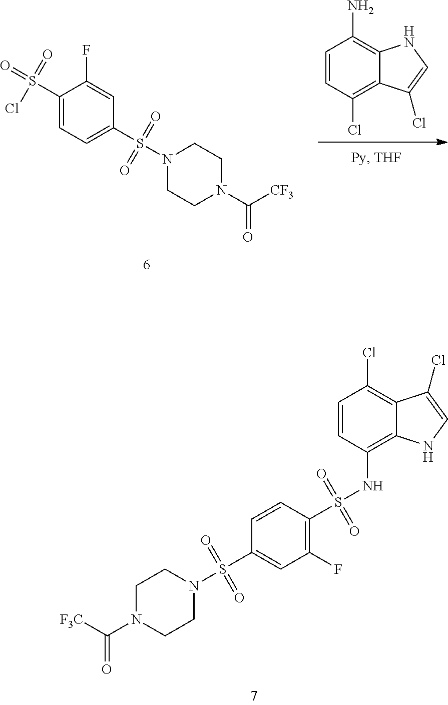 [SMILES: O=C(N1CCN(S(=O)(=O)c2ccc(S(=O)(=O)Cl)c(F)c2)CC1)C(F)(F)F ; Nc1ccc(Cl)c2c(Cl)c[nH]c12 ; O=C(N1CCN(S(=O)(=O)c2ccc(S(=O)(=O)Nc3ccc(Cl)c4c(Cl)c[nH]c34)c(F)c2)CC1)C(F)(F)F]

To a solution of 3,4-dichloro-1H-indol-7-amine (40.0 mg, 0.20 mmol) and Pyridine (54.5 mg, 0.69 mmol) in THF (3 mL) was added 2-fluoro-4-((4-(2,2,2-trifluoroacetyl)piperazin-1-yl) sulfonyl)benzene sulfonyl chloride (100.0 mg, 0.23 mmol). The mixture was stirred at rt for 18 h until the starting material was consumed completely. The reaction mixture was concentrated to give a crude product, which was purified by prep-HPLC to give N-(3,4-dichloro-1H-indol-7-yl)-2-fluoro-4-((4-(2,2,2-trifluoroacetyl)piperazin-1-yl)sulfonyl)benzenesulfonamide (40.0 mg, yield: 33.2%) a white solid. LCMS (ESI) found: 603 [M+H]⁺.

### 7. Preparation of N-(3,4-dichloro-1H-indol-7-yl)-2-fluoro-4-(piperazin-1-ylsulfonyl)benzenesulfonamide

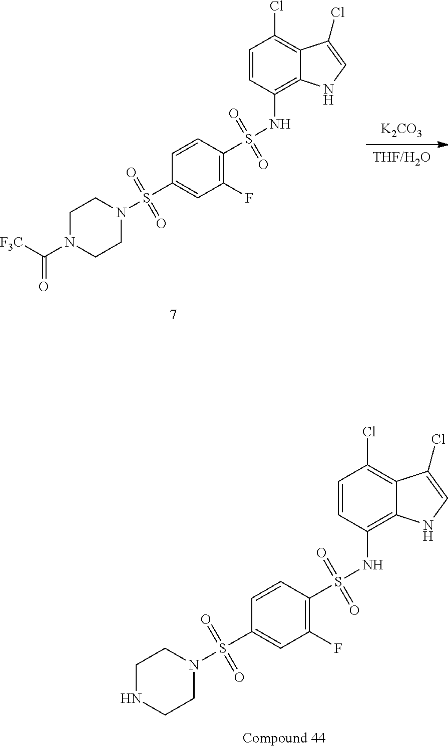

To a solution of N-(3,4-dichloro-1H-indol-7-yl)-2-fluoro-4-((4-(2,2,2-trifluoroacetyl) piperazin-1-yl)sulfonyl)benzenesulfonamide (40.0 mg, 0.066 mmol) in THF/H₂O (4/1, 10 mL) was added K₂CO₃ (27.3 mg, 0.20 mmol). The mixture was stirred at 70° C. for 18 h until the starting material was consumed completely. The mixture was filtered and concentrated to give a crude product, which was purified by prep-HPLC to give N-(3,4-dichloro-1H-indol-7-yl)-2-fluoro-4-(piperazin-1-ylsulfonyl)benzenesulfonamide (HCOOH salt) (Compound 44, 21.1 mg, yield: 45.7%) as a white solid. LCMS (ESI) found: 507 [M+H]⁺. ¹H NMR (400 MHz, DMSO-d6) δ: 11.34 (s, 1H), 8.14 (s, 0.58H), 8.04-7.97 (m, 1H), 7.66 (d, J=8.9 Hz, 1H), 7.61 (d, J=9.8 Hz, 1H), 7.29 (d, J=2.1 Hz, 1H), 6.74 (d, J=8.2 Hz, 1H), 6.68 (d, J=8.2 Hz, 1H), 3.03 (d, J=4.3 Hz, 8H).

### Example 15: Preparation of N-(3,4-dichloro-1H-indol-7-yl)-3-fluoro-4-(piperazin-1-ylsulfonyl) benzenesulfonamide: Compound 45

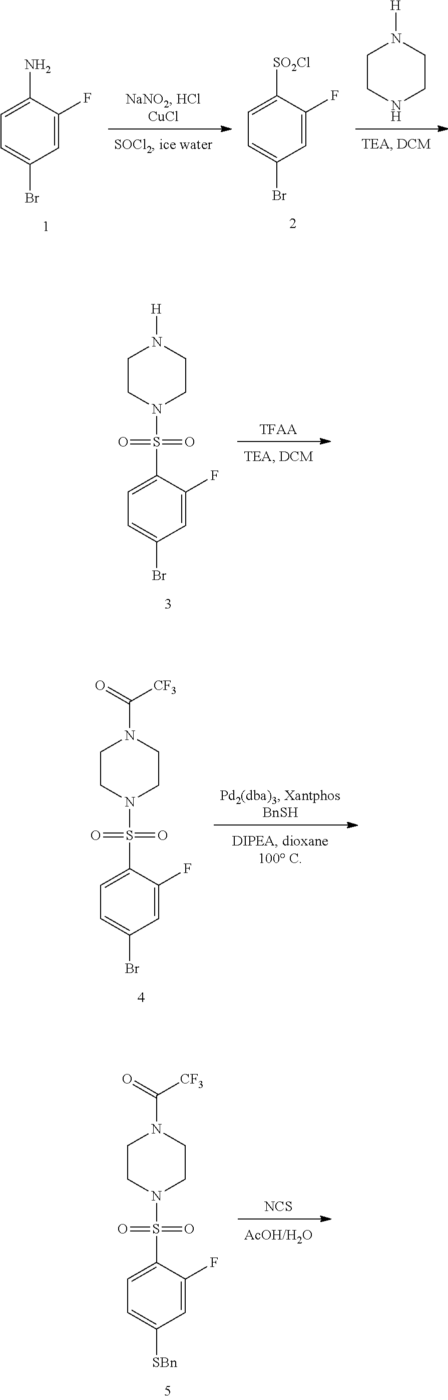

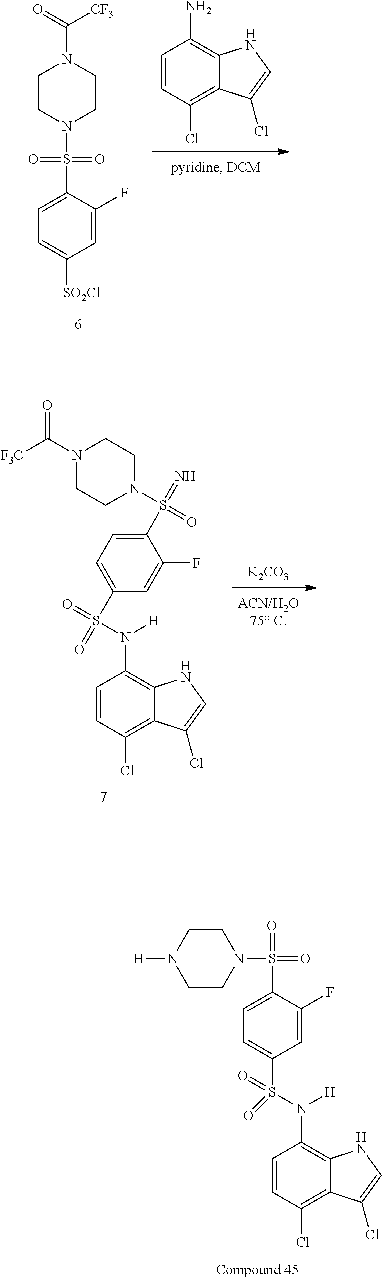

### 1. Preparation of 4-bromo-2-fluorobenzenesulfonyl chloride

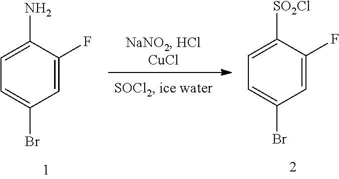

A solution of SOCl₂ (9 mL) in ice-water (45 mL) was stirred at rt for 18 h, followed by the addition of CuCl (115.0 mg, 1.15 mmol) at −5° C. The reaction mixture was stirred for 10 min to give a solution A. To a solution of 4-bromo-2-fluoroaniline (5.0 g, 26.31 mmol) in HCl (10 mol/L, 25 mL) was added NaNO₂ (2.72 g, 39.47 mmol) in H₂O (10 mL) at −10° C. and stirred for 20 min to give a solution B. The solution B was added dropwise to solution A at −5° C. and stirred for 3 h. The resulting solution was extracted with DCM (30 mL×3). The combined organic layer was washed with brine, dried over anhydrous Na₂SO₄, filtered and then concentrated to give a crude product, which was triturated with hexane to give 4-bromo-2-fluorobenzene-1-sulfonyl chloride (6.0 g, 83.4%) as a yellow solid.

### 2. Preparation of 1-((4-bromo-2-fluorophenyl)sulfonyl)piperazine

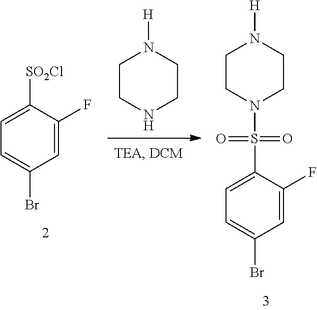

To a solution of 4-bromo-2-fluorobenzene-1-sulfonyl chloride (6.0 g, 21.94 mmol) in DCM (20 mL) were added piperazine (8.6 mL, 109.7 mmol) and TEA (6.6 g, 65.81 mmol). The mixture was stirred at rt for 2 h. The mixture was diluted with DCM (30 mL), washed with water (30 mL×2), brine (20 mL), dried over anhydrous Na₂SO₄, filtered, concentrated under reduced pressure to afford 1-(4-bromo-2-fluorobenzenesulfonyl)-4-hydrogeniopiperazine (6.2 g, 87.4%), which was used for next step directly. LCMS (ESI) found: 323 [M+H]⁺.

### 3. Preparation of 1-(4-((4-bromo-2-fluorophenyl)sulfonyl)piperazin-1-yl)-2,2,2-trifluoro ethan-1-one

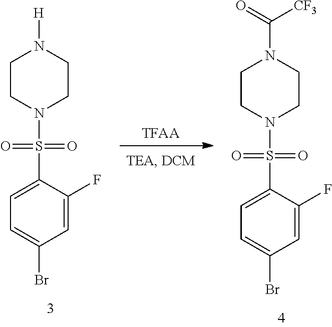 [SMILES: O=S(=O)(c1ccc(Br)cc1F)N1CCNCC1 ; "TFAA" ; O=C(N1CCN(S(=O)(=O)c2ccc(Br)cc2F)CC1)C(F)(F)F]

To a solution of 1-(4-bromo-2-fluorobenzenesulfonyl)-4-hydrogeniopiperazine (2.0 g, 6.19 mmol) in DCM (20 mL) were added TFAA (1.56 g, 7.43 mmol) and TEA (1.88 g, 18.56 mmol). The mixture was stirred at room temperature for 30 min. The mixture was diluted with water (20 mL) and extracted with DCM (20 mL×3). The combined organic layer was concentrated to give a crude product, which was purified by flash column (EA/PE=0 to 50%) to afford 1-(4-((4-bromo-2-fluorophenyl)sulfonyl)piperazin-1-yl)-2,2,2-trifluoroethan-1-one (2.4 g, 5.73 mmol, 92.52%) as white solid. LCMS (ESI) found: 419 [M+H]⁺.

### 4. Preparation of 1-(4-((4-(benzylthio)-2-fluorophenyl)sulfonyl)piperazin-1-yl)-2,2,2-trifluoroethan-1-one

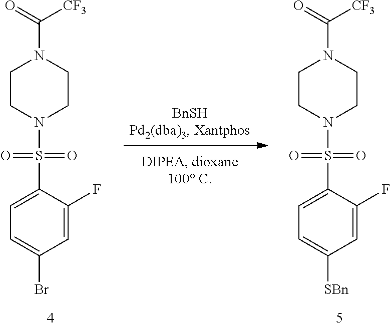

To a solution of 1-(4-((4-bromo-2-fluorophenyl)sulfonyl)piperazin-1-yl)-2,2,2-trifluoroethane-1-one (1.0 g, 2.39 mmol) in dioxane (15 mL) were added Pd₂(dba)₃ (0.22 g, 0.24 mmol), DIPEA (0.62 g, 4.77 mmol), Xantphos (0.14 g, 0.24 mmol) and BnSH (0.36 g, 2.86 mmol). The mixture was charged with N₂ for three times and stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give 1-(4-((4-(benzylthio)-2-fluorophenyl)sulfonyl)piperazin-1-yl)-2,2,2-trifluoroethan-1-one (1.0 g, 90.6%) as a yellow solid. LCMS (ESI) found: 463 [M+H]⁺.

### 5. Preparation of 3-fluoro-4-((4-(2,2,2-trifluoroacetyl)piperazin-1-yl)sulfonyl)benzene sulfonyl chloride

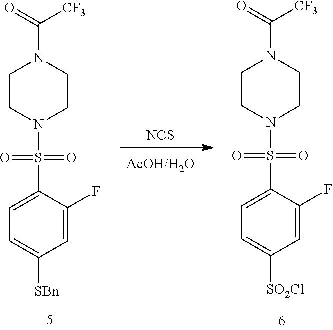

To a solution of 1-(4-((4-(benzylthio)-2-fluorophenyl)sulfonyl)piperazin-1-yl)-2,2,2-trifluoro ethan-1-one (500.0 mg, 1.08 mmol) in AcOH/H₂O (9/1, 4 mL) was added NCS (612.4 mg, 4.54 mmol). The mixture was stirred at rt for 2 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 3-fluoro-4-((4-(2,2,2-trifluoroacetyl)piperazin-1-yl)sulfonyl)benzenesulfonyl chloride (350.0 mg, 73.8%) as a white solid. LCMS (ESI) found: 439 [M+H]⁺.

### 6. Preparation of N-(3,4-dichloro-1H-indol-7-yl)-3-fluoro-4-(4-(2,2,2-trifluoroacetyl)piperazine-1-sulfonimidoyl)benzenesulfonamide

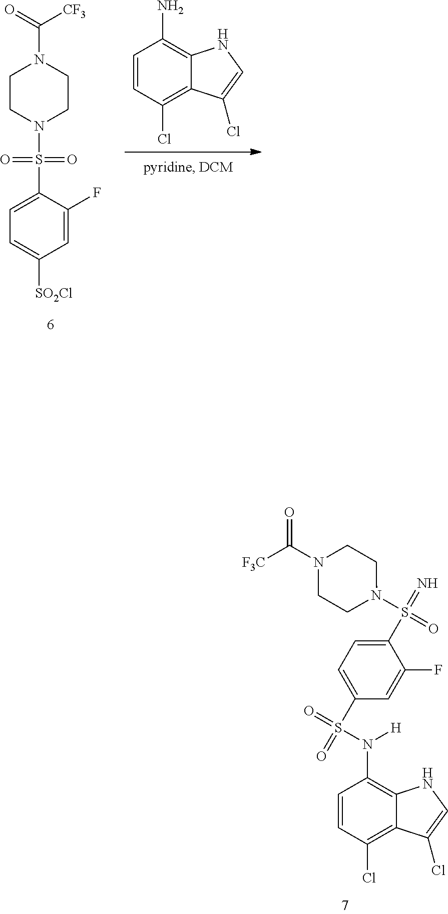 [SMILES: O=C(N1CCN(S(=O)(=O)c2ccc(S(=O)(=O)Cl)cc2F)CC1)C(F)(F)F ; Nc1ccc(Cl)c2c(Cl)c[nH]c12 ; N=S(=O)(c1ccc(S(=O)(=O)Nc2ccc(Cl)c3c(Cl)c[nH]c23)cc1F)N1CCN(C(=O)C(F)(F)F)CC1]

To a solution of 3,4-dichloro-1H-indol-7-amine (22.9 mg, 0.11 mmol) and Pyridine (26.1 mg, 0.33 mmol) in DCM (5 mL) was added 3-fluoro-4-((4-(2,2,2-trifluoroacetyl)piperazin-1-yl) sulfonyl)benzenesulfonyl chloride (50.0 mg, 0.11 mmol). The mixture was stirred at room temperature for 30 min. The mixture was concentrated and the residue was purified by flash column to afford N-(3,4-dichloro-1H-indol-7-yl)-3-fluoro-4-(4-(2,2,2-trifluoroacetyl)piperazine-1-sulfon imidoyl)benzenesulfonamide (41.0 mg, 59.6%) as a white solid. LCMS (ESI) found: 602 [M+H]⁺.

### 7. Preparation of N-(3,4-dichloro-1H-indol-7-yl)-3-fluoro-4-(piperazin-1-ylsulfonyl) benzenesulfonamide

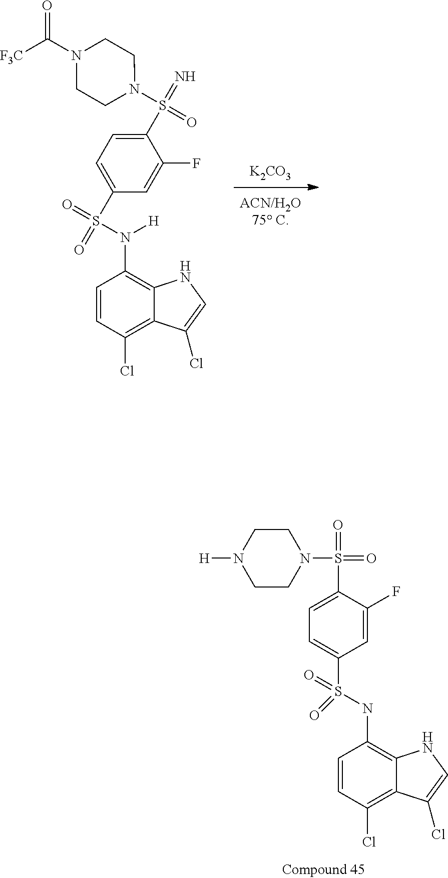

To a solution of N-(3,4-dichloro-1H-indol-7-yl)-3-fluoro-4-(4-(2,2,2-trifluoroacetyl)piperazine-1-sulfonimidoyl)benzenesulfonamide (41.0 mg, 0.07 mmol) in ACN/H₂O (4/1, 4 mL) was added K₂CO₃ (29.0 mg, 0.21 mmol). The mixture was stirred at 75° C. for 3 h. The mixture was cooled down to room temperature, filtered and concentrated to give a crude product, which was purified by Prep-HPLC to afford N-(3,4-dichloro-1H-indol-7-yl)-3-fluoro-4-(piperazin-1-ylsulfonyl)benzenesulfonamide (HCOOH salt) (Compound 45, 11.0 mg, 31.9%) as a white solid. LCMS (ESI) found: 507 [M+H]⁺. ¹H NMR (400 MHz, DMSO-d6) δ 11.50 (s, 1H), 8.14 (s, 0.56H), 7.94-7.87 (m, 1H), 7.81 (d, J=9.7 Hz, 1H), 7.73 (dd, J=8.2, 1.6 Hz, 1H), 7.45 (s, 1H), 6.87 (d, J=8.2 Hz, 1H), 6.68 (d, J=8.2 Hz, 1H), 3.24-3.19 (m, 4H), 3.15-3.09 (m, 4H).

### Example 16: Preparation of N-(3,4-dichloro-1H-indol-7-yl)-4-((2-methylpiperazin-1-yl)sulfonyl)benzene sulfonamide: Compound 46

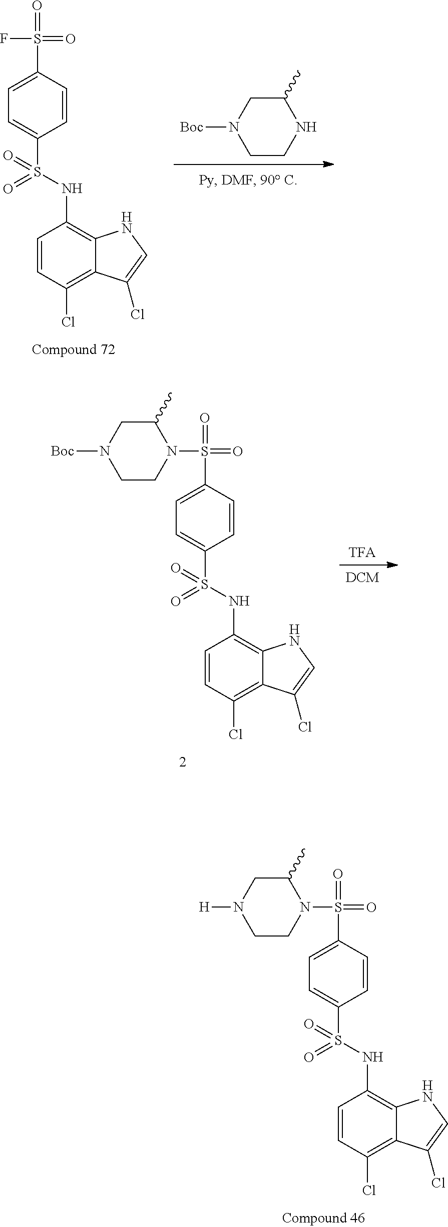

### 1. Preparation of tert-butyl 4-((4-(N-(3,4-dichloro-1H-indol-7-yl)sulfamoyl)phenyl) sulfonyl)-3-methylpiperazine-1-carboxylate

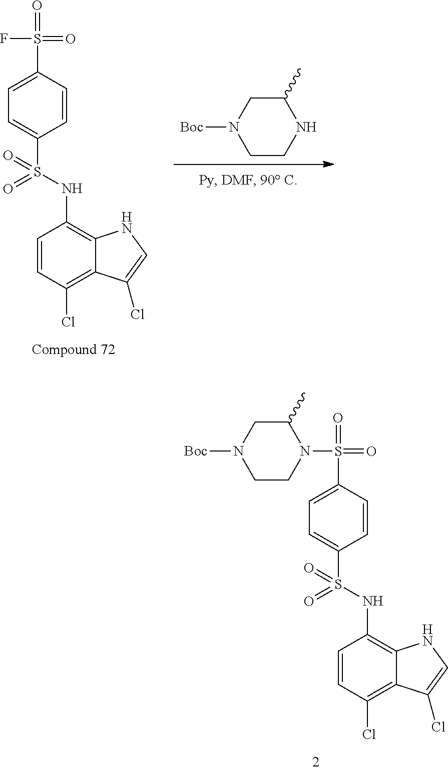

To a solution of 4-(N-(3,4-dichloro-1H-indol-7-yl)sulfamoyl)benzenesulfonyl fluoride (30.0 mg, 0.07 mmol) and pyridine (16.6 mg, 0.21 mmol) in DMF (2 mL) was added tert-butyl 3-methylpiperazine-1-carboxylate (16.0 mg, 0.08 mmol). The mixture was stirred at 90° C. for 18 h. The mixture was concentrated to give a crude product, which was purified by column chromatography on silica gel eluting with PE/EA to give tert-butyl 4-((4-(N-(3,4-dichloro-1H-indol-7-yl)sulfamoyl)phenyl)sulfonyl)-3-methylpiperazine-1-carboxylate (20.0 mg, yield: 47.4%) as a white solid. LCMS (ESI) found: 603 [M+H]⁺.

### 2. Preparation of N-(3,4-dichloro-1H-indol-7-yl)-4-((2-methylpiperazin-1-yl)sulfonyl) benzenesulfonamide

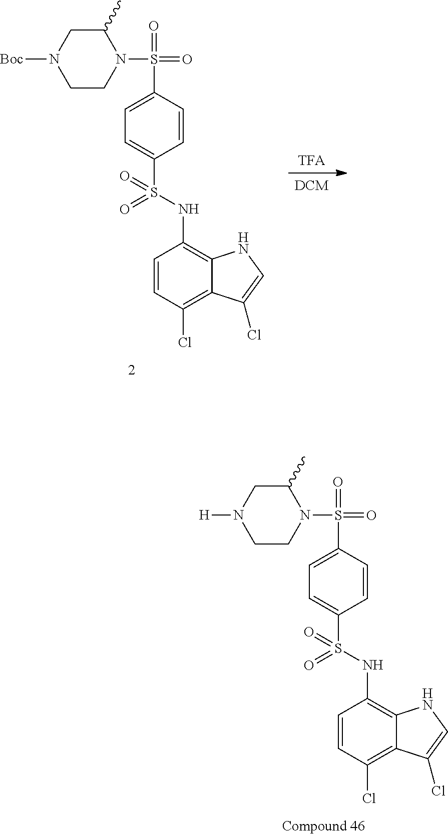

To a solution of tert-butyl 4-((4-(N-(3,4-dichloro-1H-indol-7-yl)sulfamoyl)phenyl) sulfonyl)-3-methylpiperazine-1-carboxylate (20.0 mg, 0.033 mmol) in DCM (2 mL) was added TFA (0.5 mL). The mixture was stirred at rt for 2 h. The mixture was concentrated and basified with 10% aqueous Na₂CO₃ (20 mL). The mixture was extracted with DCM (20 mL×3). The combined organic layer was concentrated to give a crude product, which was purified to by prep-HPLC to give N-(3,4-dichloro-1H-indol-7-yl)-4-((2-methylpiperazin-1-yl)sulfonyl) benzenesulfonamide (HCOOH salt) (Compound 46, 6.2 mg, yield: 37.3%) as a white solid. LCMS (ESI) found: 503 [M+H]⁺. ¹H NMR (400 MHz, DMSO-d6) δ 11.41 (s, 1H), 8.14 (s, 0.44H), 7.89 (q, J=8.7 Hz, 4H), 7.46 (s, 1H), 6.84 (d, J=8.2 Hz, 1H), 6.54 (d, J=8.2 Hz, 1H), 3.93-3.87 (m, 1H), 3.51-3.47 (m, 1H), 3.05-3.01 (m, 1H), 2.85 (d, J=12.5 Hz, 1H), 2.69 (d, J=12.6 Hz, 1H), 2.59-2.55 (m, 1H), 2.46-2.41 (m, 1H), 0.98 (d, J=6.8 Hz, 3H).

### Example 17: Preparation of N-(3,4-dichloro-1H-indol-7-yl)-4-((2-propylpiperazin-1-yl)sulfonyl) benzenesulfonamide; Compound 47

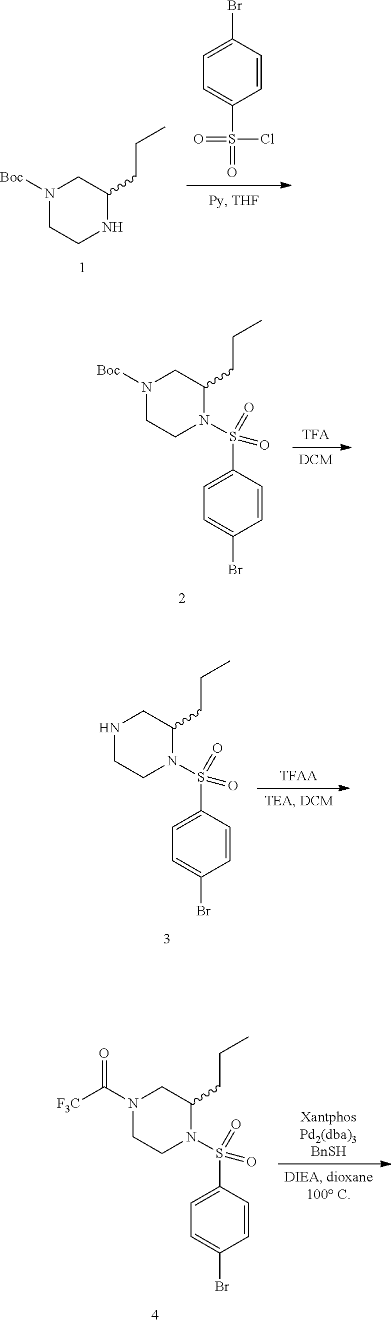

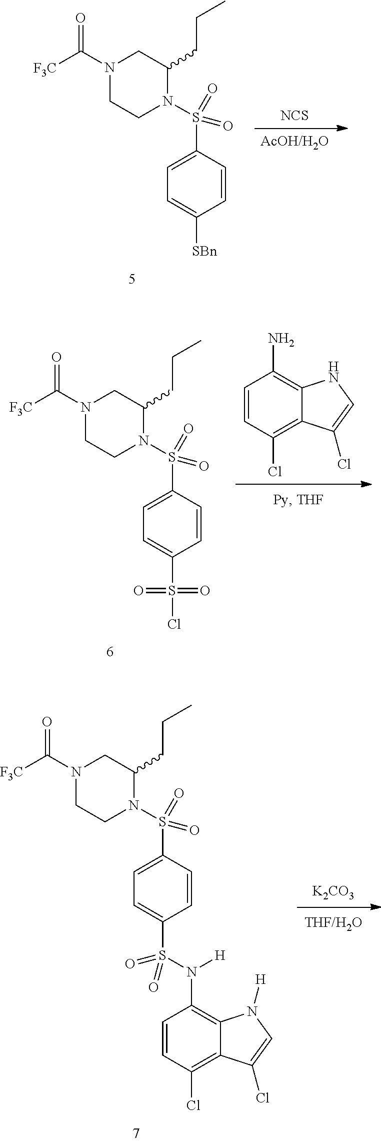

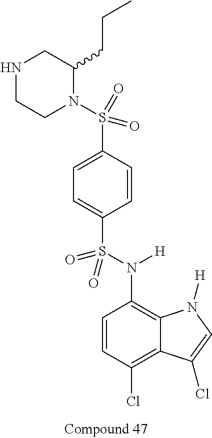 [SMILES: CCCC1CNCCN1S(=O)(=O)c1ccc(S(=O)(=O)Nc2ccc(Cl)c3c(Cl)c[nH]c23)cc1]

### 1. Preparation of tert-butyl 4-((4-bromophenyl)sulfonyl)-3-propylpiperazine-1-carboxylate

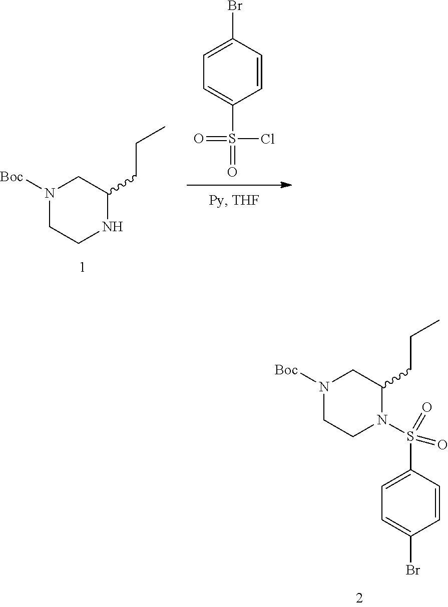

To a solution of tert-butyl 3-propylpiperazine-1-carboxylate (200 mg, 0.88 mmol) in DCM (5 mL) were added pyridine (208.6 mg, 2.64 mmol) and 4-bromobenzenesulfonyl chloride (224.4 mg, 0.88 mmol). The reaction mixture was stirred for 1 h at rt. The reaction mixture was diluted with water (20 mL) and extracted with EA (50 mL×2). The organic layer was washed with water (20 mL), brine (20 mL), dried over Na₂SO₄ and concentrated under reduced pressure to afford the crude product, which was purified by column chromatography on silica gel eluted with PE/EA (10/1 to 2/1) to give tert-butyl 4-((4-bromophenyl)sulfonyl)-3-propylpiperazine-1-carboxylate (280.0 mg, 91.7%) as a white solid. LCMS (ESI) found: 347 [M+H]⁺.

### 2. Preparation of 1-((4-bromophenyl)sulfonyl)-2-propylpiperazine

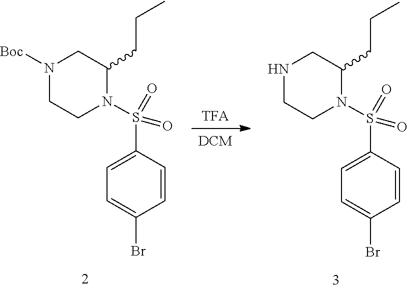

To a solution of tert-butyl 4-((4-bromophenyl)sulfonyl)-3-propylpiperazine-1-carboxylate (280.0 mg, 0.81 mmol) in DCM (2 mL) was added TFA (1 mL). The reaction mixture was stirred for 1 h at rt. The reaction mixture was concentrated, basified with aqueous NaHCO₃ (10 mL), diluted with water (20 mL) and extracted with DCM (20 mL×3). The combined organic layer was dried over MgSO₄, filtered and concentrated to give crude 1-((4-bromophenyl)sulfonyl)-2-propylpiperazine (200.0 mg, crude) as a brown oil which was used directly for the next step. LCMS (ESI) found: 247 [M+H]⁺.

### 3. Preparation of 1-(4-((4-bromophenyl)sulfonyl)-3-propylpiperazin-1-yl)-2,2,2-trifluoroethan-1-one

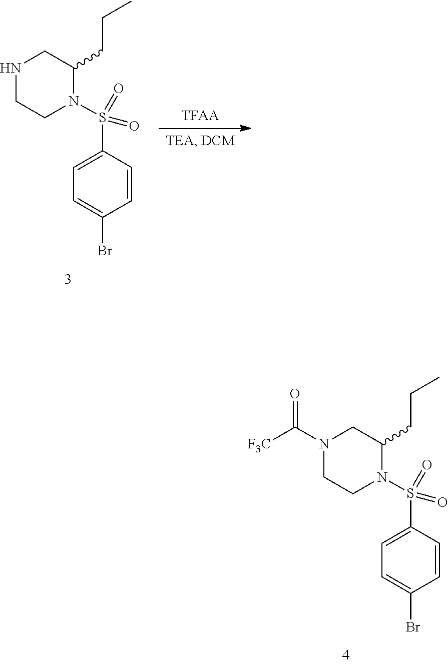 [SMILES: CCCC1CNCCN1S(=O)(=O)c1ccc(Br)cc1 ; "TFAA" ; "TEA, DCM" ; CCCC1CN(C(=O)C(F)(F)F)CCN1S(=O)(=O)c1ccc(Br)cc1]

To a solution of 1-((4-bromophenyl)sulfonyl)-2-propylpiperazine (200.0 mg, 0.81 mmol) in DCM (5 mL) was added TEA (245.3 mg, 2.43 mmol) and TFAA (204.1 mg, 0.97 mmol). The reaction mixture was stirred for 2 h at rt. The reaction mixture was diluted with water (25 mL) and extracted with DCM (25 mL×3). The organic layer was washed with water (20 mL), brine (20 mL), dried over Na₂SO₄ and concentrated under reduced pressure to afford a crude product, which was purified by column chromatography on silica gel eluted with DCM/MEOH (pure DCM to 10/1) to give 1-(4-((4-bromophenyl)sulfonyl)-3-propylpiperazin-1-yl)-2,2,2-trifluoroethan-1-one (240.0 mg, 66.9%) as a white solid. LCMS (ESI) found: 443 [M+H]⁺.

### 4. Preparation of 1-(4-((4-(benzylthio)phenyl)sulfonyl)-3-propylpiperazin-1-yl)-2,2,2-trifluoroethan-1-one

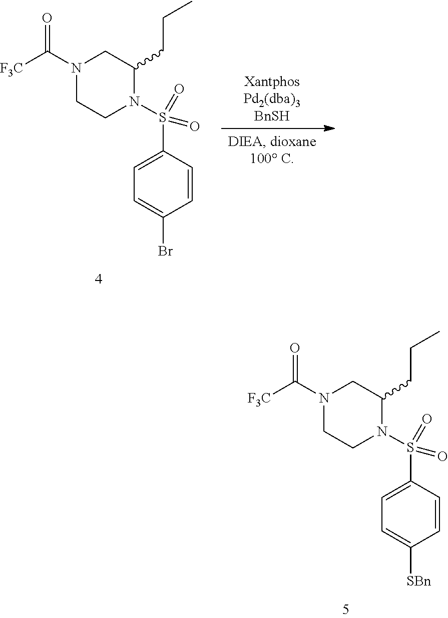

A mixture of 1-(4-((4-bromophenyl)sulfonyl)-3-propylpiperazin-1-yl)-2,2,2-trifluoroethan-1-one (240.0 mg, 0.54 mmol) and DIEA (174.1 mg, 1.35 mmol) in dioxane (3 mL) were added BnSH (80.3 mg, 0.65 mmol), Pd₂(dba)₃ (45.8 mg, 0.05 mmol) and Xantphos (57.8 mg, 0.10 mmol). The mixture was degassed with N₂ for 3 times and then stirred for 3 h at 100° C. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give 1-(4-((4-(benzylthio)phenyl)sulfonyl)-3-propylpiperazin-1-yl)-2,2,2-trifluoroethan-1-one (223.0 mg, 84.8%) as a white solid. LCMS (ESI) found: 487 [M+H]⁺.

### 5. Preparation of 4-((2-propyl-4-(2,2,2-trifluoroacetyl)piperazin-1-yl)sulfonyl) benzenesulfonyl chloride

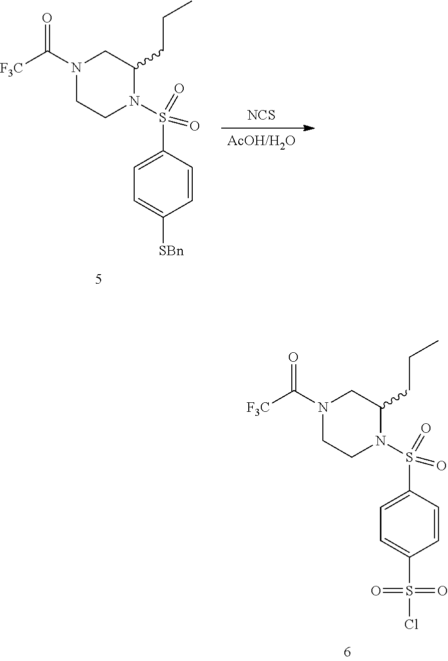

To a solution of 1-(4-((4-(benzylthio)phenyl)sulfonyl)-3-propylpiperazin-1-yl)-2,2,2-trifluoroethan-1-one (200.0 mg, 0.41 mmol) in AcOH/H₂O (9/1, 3 mL) was added NCS (232.8 mg, 1.72 mmol). The mixture was stirred at rt for 2 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 4-((2-propyl-4-(2,2,2-trifluoroacetyl)piperazin-1-yl)sulfonyl)benzenesulfonyl chloride (155.0 mg, 61.4%) as a white solid. LCMS (ESI) found: 463 [M+H]⁺.

### 6. Preparation of N-(3,4-dichloro-1H-indol-7-yl)-4-((2-propyl-4-(2,2,2-trifluoroacetyl)piperazin-1-yl)sulfonyl)benzenesulfonamide

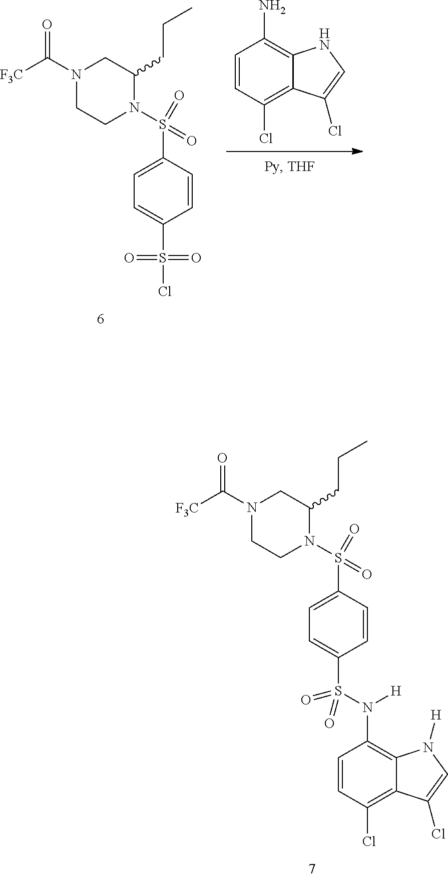 [SMILES: CCCC1CN(C(=O)C(F)(F)F)CCN1S(=O)(=O)c1ccc(S(=O)(=O)Cl)cc1 ; Nc1ccc(Cl)c2c(Cl)c[nH]c12 ; CCCC1CN(C(=O)C(F)(F)F)CCN1S(=O)(=O)c1ccc(S(=O)(=O)Nc2ccc(Cl)c3c(Cl)c[nH]c23)cc1]

To a solution of 3,4-dichloro-1H-indol-7-amine (58.3 mg, 0.29 mmol) and pyridine (68.7 mg, 0.87 mmol) in THF (2 mL) was added 4-((2-propyl-4-(2,2,2-trifluoroacetyl)piperazin-1-yl)sulfonyl)benzenesulfonyl chloride (135.0 mg, 0.29 mmol). The mixture was stirred at rt for 4 h. The mixture was concentrated to give a crude product. which was purified by prep-TLC eluted with DCM/MeOH=15/1 to give N-(3,4-dichloro-1H-indol-7-yl)-4-((2-propyl-4-(2,2,2-trifluoroacetyl)piperazin-1-yl)sulfonyl)benzenesulfonamide (160.0 mg, 46.4%) as a white solid. LCMS (ESI) found: 627 [M+H]⁺.

### 7. Preparation of N-(3,4-dichloro-1H-indol-7-yl)-4-((2-propylpiperazin-1-yl)sulfonyl)benzenesulfonamide

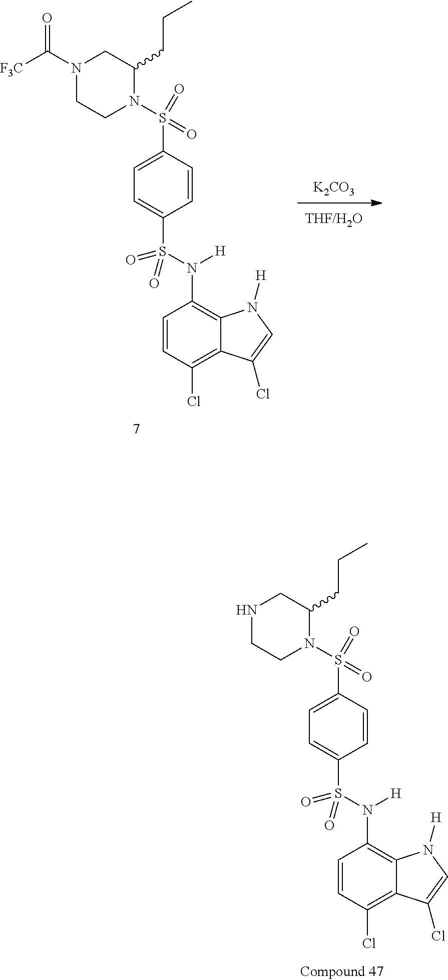 [SMILES: CCCC1CN(C(=O)C(F)(F)F)CCN1S(=O)(=O)c1ccc(S(=O)(=O)Nc2ccc(Cl)c3c(Cl)c[nH]c23)cc1 ; "K2CO3" ; CCCC1CNCCN1S(=O)(=O)c1ccc(S(=O)(=O)Nc2ccc(Cl)c3c(Cl)c[nH]c23)cc1]

To a solution of N-(3,4-dichloro-1H-indol-7-yl)-4-((2-propyl-4-(2,2,2-trifluoroacetyl)piperazin-1-yl)sulfonyl)benzenesulfonamide (80.0 mg, 0.13 mmol) in THF/H₂O (511, 2 mL) was added K₂CO₃ (52.8 mg, 0.38 mmol). The mixture was stirred at 50° C. overnight. The mixture was filtered and the filtrate was concentrated to give a crude product, which was purified by prep-TLC eluted with DCM/MeOH=15/1 to give N-(3,4-dichloro-1H-indol-7-yl)-4-((2-propylpiperazin-1-yl)sulfonyl)benzenesulfonamide (HCOOH salt) (Compound 47, 45.4 mg, 65.8%) as a white solid. LCMS (ESI) found: 531 [M+H]⁻. ¹H NMR (400 MHz, DMSO-d6) δ 11.48 (s, 1H), 8.14 (s, 0.56H), 7.96 (d, J=8.2, 2H), 7.88 (d, J=8.3, 2H), 7.51 (s, 1H), 6.89 (d, J=8.1, 1H), 6.54 (d, J=8.1, 1H), 3.81 (s, 1H), 3.67-3.61 (m, 1H), 3.13-3.05 (m, 1H), 2.84-2.76 (m, 2H), 2.44-2.39 (m, 1H), 2.27-2.18 (m, 1H), 1.53-1.47 (m, 2H), 1.22-1.14 (m, 2H), 0.80 (t, J=7.3, 3H).

### Example 18: Preparation of N-(3,4-dichloro-1H-indol-7-yl)-4-((4-methylpiperazin-1-yl)sulfonyl)benzenesulfonamide; Compound 48

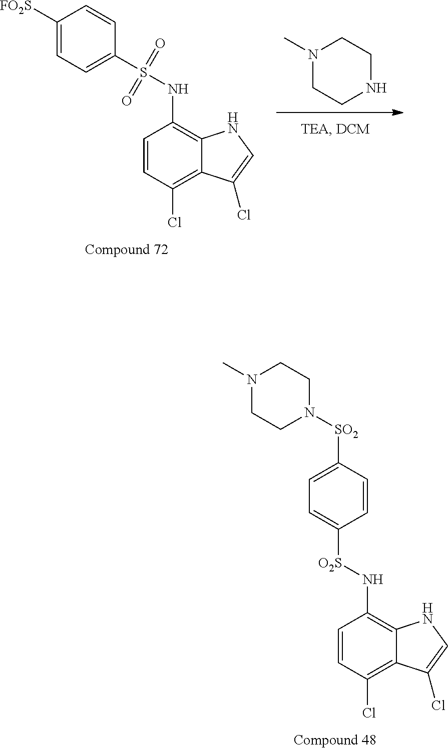

### 1. Preparation of N-(3,4-dichloro-1H-indol-7-yl)-4-((4-methylpiperazin-1-yl)sulfonyl) benzenesulfonamide

To a solution of 4-(N-(3,4-dichloro-1H-indol-7-yl)sulfamoyl)benzenesulfonyl fluoride (30.0 mg, 0.07 mmol) and TEA (21.5 mg, 0.21 mmol) in DCM (2 mL) was added 1-methylpiperazine (8.4 mg, 0.08 mmol). The mixture was stirred at room temperature for 18 h. The mixture was concentrated to give a crude product, which was purified by column chromatography on silica gel eluting with PE/EA to give N-(3,4-dichloro-1H-indol-7-yl)-4-((4-methylpiperazin-1-yl) sulfonyl)benzenesulfonamide (Compound 48, 5.5 mg, yield: 15.6%) as a white solid. LCMS (ESI) found: 503 [M+H]⁺. ¹H NMR (400 MHz, DMSO-d6) δ 11.36 (s, 1H), 10.17 (s, 1H), 7.91-7.83 (m, 4H), 7.49 (s, 1H), 6.92 (d, J=8.1 Hz, 1H), 6.58 (d, J=8.2 Hz, 1H), 2.88 (s, 4H), 2.35 (s, 4H), 2.16 (s, 3H).

### Example 19: Preparation of ethyl 4-((4-(N-(3,4-dichloro-1H-indol-7-yl)sulfamoyl)phenyl)sulfonyl) piperazine-1-carboxylate; Compound 49

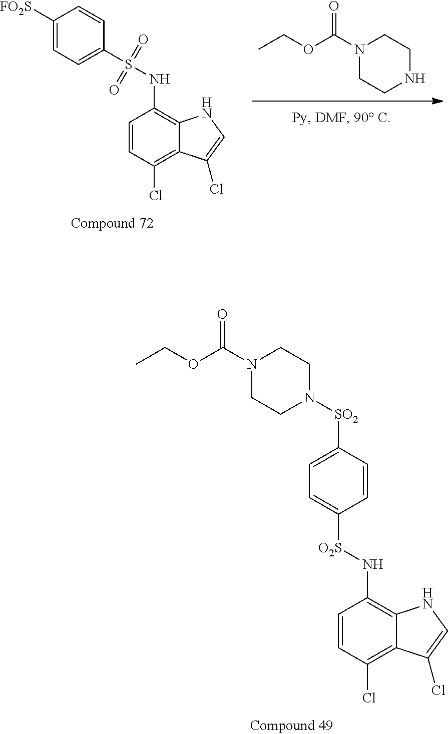

### 1. Preparation of ethyl 4-((4-(N-(3,4-dichloro-1H-indol-7-yl)sulfamoyl)phenyl)sulfonyl) piperazine-1-carboxylate

To a solution of 4-(N-(3,4-dichloro-1H-indol-7-yl)sulfamoyl)benzenesulfonyl fluoride (30.0 mg, 0.07 mmol) and Py. (16.6 mg, 0.21 mmol) in DMF (2 mL) was added ethyl piperazine-1-carboxylate (12.6 mg, 0.08 mmol) at room temperature. The mixture was stirred for 18 h at 90° C. The mixture was concentrated and purified to afford a crude product, which was purified by column chromatography on silica gel eluting with PE/EA to give ethyl 4-((4-(N-(3,4-dichloro-1H-indol-7-yl)sulfamoyl)phenyl)sulfonyl)piperazine-1-carboxylate (Compound 49, 6.2 mg, yield: 15.8%) as a white solid. LCMS (ESI) found: 561 [M+H]⁺. ¹H NMR (400 MHz, DMSO) δ 11.42 (s, 1H), 10.21 (s, 1H), 7.92-7.86 (m, 4H), 7.49 (s, 1H), 6.91 (d, J=8.1 Hz, 1H), 6.56 (d, J=8.1 Hz, 1H), 4.00 (q, J=7.1 Hz, 2H), 3.49-3.43 (m, 4H), 2.99-2.91 (m, 4H), 1.15 (t, J=7.1 Hz, 3H).

### Example 20: Preparation of 4-((4-(3-(4-cyanophenyl)propyl)piperazin-1-yl)sulfonyl)-N-(3,4-dichloro-1H-indol-7-yl)benzenesulfonamide; Compound 50

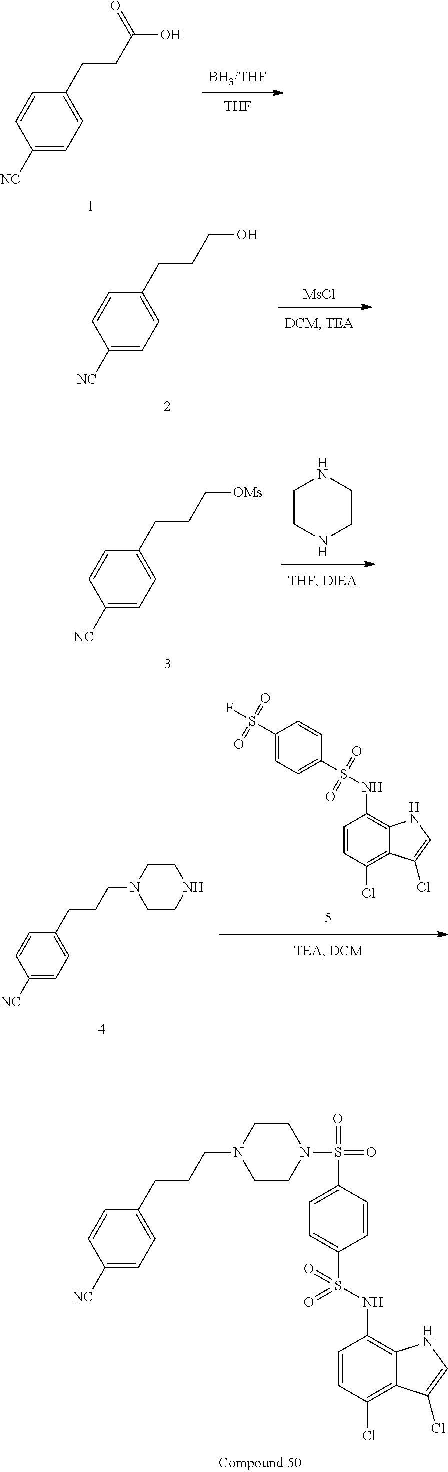

### 1. Preparation of 4-(3-hydroxypropyl)benzonitrile

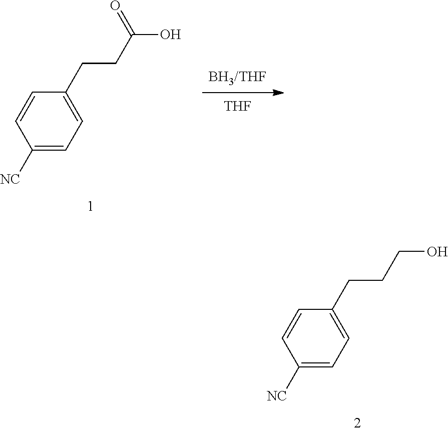 [SMILES: N#Cc1ccc(CCC(=O)O)cc1 ; "BH3/THF" ; N#Cc1ccc(CCCO)cc1 ; "THF"]

To a solution of 3-(4-cyanophenyl)propanoic acid (500.0 mg, 2.85 mmol) in THF (5 mL) was added BH₃-THF (1.9 mL) (2M in THF). The mixture was stirred at rt overnight. The reaction was quenched by slow addition of MeOH (2 mL). The reaction mixture was stirred for additional 30 min at 20° C. The reaction mixture was concentrated and purified by flash chromatography to give 4-(3-hydroxypropyl)benzonitrile (410.0 mg, 89.1%) as a light yellow oil. LCMS (ESI) found: 162 [M+H]⁺.

### 2. Preparation of 3-(4-cyanophenyl)propyl methanesulfonate

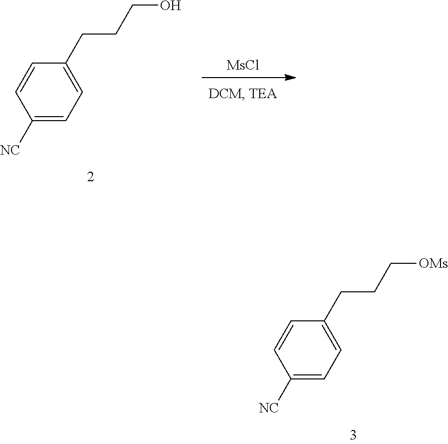 [SMILES: "MsCl" ; N#Cc1ccc(CCCO)cc1 ; CS(=O)(=O)OCCCc1ccc(C#N)cc1]

To a solution of 4-(3-hydroxypropyl)benzonitrile (410.0 mg, 2.54 mmol) in DCM (5 mL) were added TEA (514.7 mg, 5.09 mmol) and MsCl (347.9 mg, 3.05 mmol). The mixture was stirred at rt for 2 h. TLC showed the reaction has completed. The reaction was quenched with water. The mixture was concentrated and purified by flash column to give 3-(4-cyanophenyl)propyl methanesulfonate (572.0 mg, 94.0%) as a yellow solid. LCMS (ESI) found: 240 [M+H]⁺.

### 3. Preparation of 4-(3-(piperazin-1-yl)propyl)benzonitrile

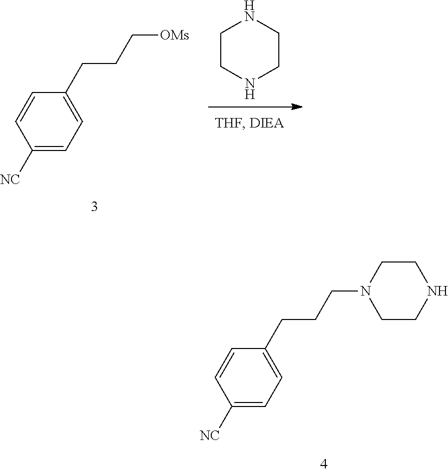 [SMILES: CS(=O)(=O)OCCCc1ccc(C#N)cc1 ; C1CNCCN1 ; N#Cc1ccc(CCCN2CCNCC2)cc1]

To a solution of piperazine (593.9 mg, 6.90 mmol) and DIPEA (177.9 mg, 1.38 mmol) in THF (10 mL) was added 3-(4-cyanophenyl)propyl methanesulfonate (330.0 mg, 1.38 mmol). The mixture was stirred at 50° C. overnight. The mixture was concentrated and purified by flash to give 4-(3-(piperazin-1-yl)propyl)benzonitrile (202.0 mg, 63.9%) as a yellow solid. LCMS (ESI) found: 230 [M+H]⁺.

### 4. Preparation of 4-((4-(3-(4-cyanophenyl)propyl)piperazin-1-yl)sulfonyl)-N-(3,4-dichloro-1H-indol-7-yl)benzenesulfonamide)

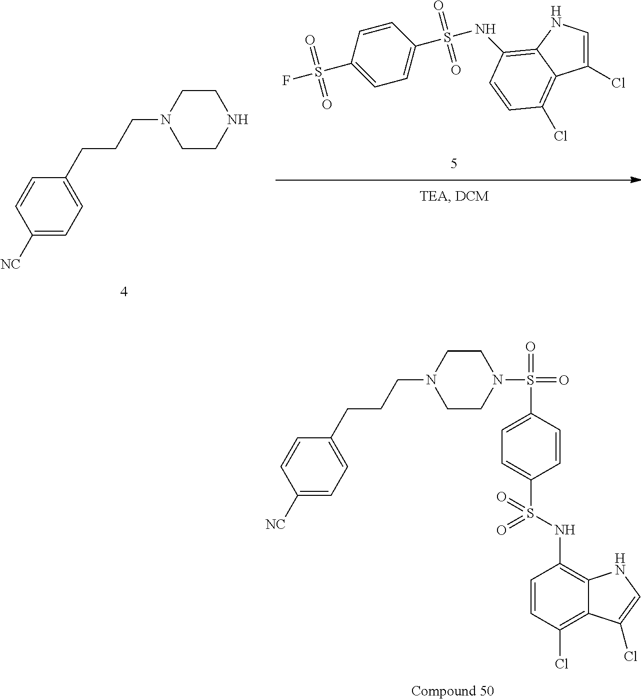 [SMILES: N#Cc1ccc(CCCN2CCNCC2)cc1 ; O=S(=O)(F)c1ccc(S(=O)(=O)Nc2ccc(Cl)c3c(Cl)c[nH]c23)cc1 ; N#Cc1ccc(CCCN2CCN(S(=O)(=O)c3ccc(S(=O)(=O)Nc4ccc(Cl)c5c(Cl)c[nH]c45)cc3)CC2)cc1]

To a solution of 4-(3-(piperazin-1-yl)propyl)benzonitrile (27.1 mg, 0.12 mmol) and TEA (12.0 mg, 0.12 mmol) in DCM (2 mL) was added 4-(N-(3,4-dichloro-1H-indol-7-yl)sulfamoyl)benzenesulfonyl fluoride (25.0 mg, 0.06 mmol). The mixture was stirred at rt overnight. TLC showed the reaction was completed. The mixture was concentrated and purified by pre-TLC to give 4-((4-(3-(4-cyanophenyl)propyl)piperazin-1-yl)sulfonyl)-N-(3,4-dichloro-1H-indol-7-yl)benzenesulfonamide (Compound 50, 14.0 mg, 37.5%) as a white solid. LCMS (ESI) found: 632 [M+H]⁺. ¹H NMR (400 MHz, DMSO-d6) δ 11.42 (s, 1H), 10.21 (s, 1H), 7.96-7.78 (m, 4H), 7.71 (d, J=8.2 Hz, 2H), 7.53 (d, J=2.7 Hz, 1H), 7.38 (d, J=8.2 Hz, 2H), 6.93 (d, J=8.2 Hz, 1H), 6.54 (d, J=8.1 Hz, 1H), 2.88 (s, 4H), 2.66-2.59 (m, 2H), 2.41 (s, 4H), 2.27 (t, J=7.0 Hz, 2H), 1.78-1.63 (m, 2H).

### Example 21: Preparation of N-(3,4-dichloro-1H-indol-7-yl)-4-((4-(2-(2-(pyridin-2-yl)ethoxy)ethyl)piperazin-1-yl)sulfonyl)benzenesulfonamide; Compound 51

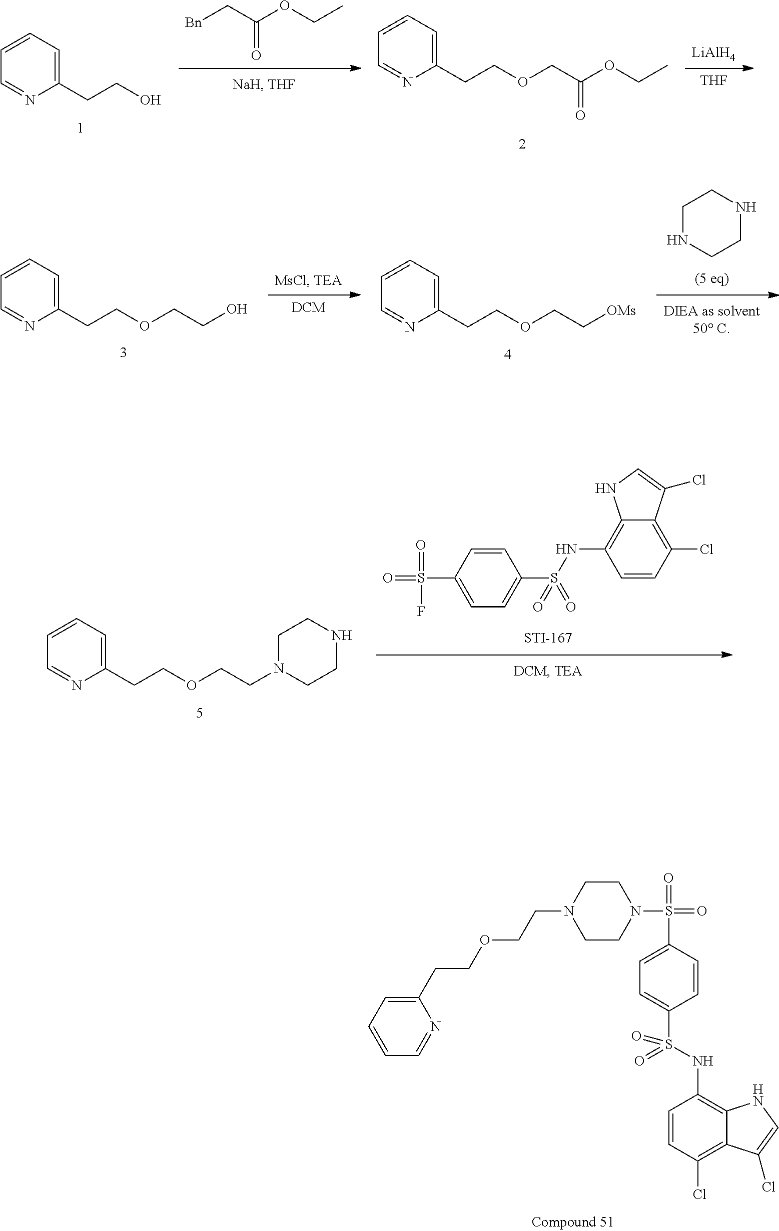

### 1. Preparation of ethyl 2-(2-(pyridin-2-yl)ethoxy)acetate

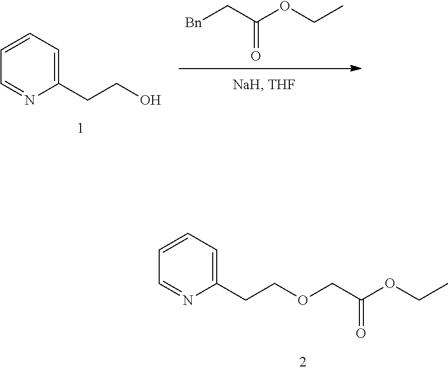

To a solution of 2-(pyridin-2-yl)ethan-1-ol (800.0 mg, 6.50 mmol) in dry THF (10 mL) was added NaH (311.8 mg, 7.80 mmol) at 0° C. After stirring for 0.5 h, ethyl 2-bromoacetate (1.3 g, 7.80 mmol) was added and stirred at rt for another 1.5 h. LCMS showed the desired product was detected. The mixture was poured into NH₄Cl aqueous (50 mL) and extracted with EA (50 mL×3). The combined organic layer was washed with brine, dried over anhydrous Na₂SO₄ and concentrated to give a crude product, which was purified by column to give ethyl 2-(2-(pyridin-2-yl)ethoxy)acetate (550.0 mg, 40.5%) as an oil. LCMS (ESI) found: 210 [M+H]⁺.

### 2. Preparation of 2-(2-(pyridin-2-yl)ethoxy)ethan-1-ol

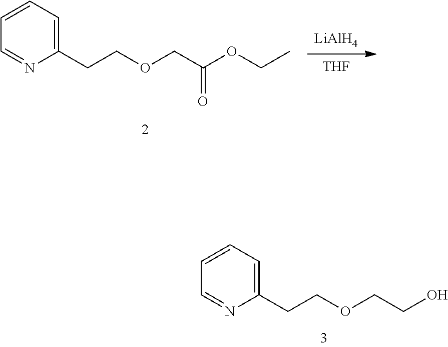

To a solution of ethyl 2-(2-(pyridin-2-yl)ethoxy)acetate (500.0 mg, 2.40 mmol) in THF (10 mL) was added LiAlH₄ (99.9 mg, 2.63 mmol) and stirred at rt for 1 h. TLC showed the reaction was completed. The mixture was poured into 0.1 M aqueous HCl (50 mL) and extracted with EA (50 mL×3), the combined organic layer was washed with brine, dried over anhydrous Na₂SO₄, and concentrated to give a crude product which was purified by column to give 2-(2-(pyridin-2-yl)ethoxy)ethan-1-ol (350.0 mg, 87.6%) as an oil. LCMS (ESI) found: 168 [M+H]⁺.

### 3. Preparation of 2-(2-(pyridin-2-yl)ethoxy)ethyl methanesulfonate

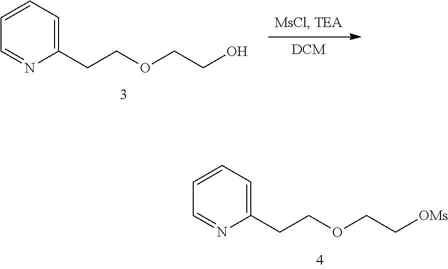

To a solution of 2-(2-(pyridin-2-yl)ethoxy)ethan-1-ol (300.0 mg, 1.79 mmol) and TEA (542.4 mg, 5.37 mmol) in dry DCM (5 mL) was added MsCl (410.9 mg, 3.56 mmol) at 0° C. the resulting reaction mixture was stirred at rt for 30 min. TLC showed the reaction was completed. The mixture was poured into water (50 mL) and extracted with EA (50 mL×3). The combined organic layer was washed with brine, dried over anhydrous Na₂SO₄ and concentrated to give a crude 2-(2-(pyridin-2-yl)ethoxy)ethyl methanesulfonate (360.0 mg, 81.8%) which was used directly in the next step without further purification. LCMS (ESI) found: 246 [M+H]⁺.

### 4. Preparation of 1-(2-(2-(pyridin-2-yl)ethoxy)ethyl)piperazine

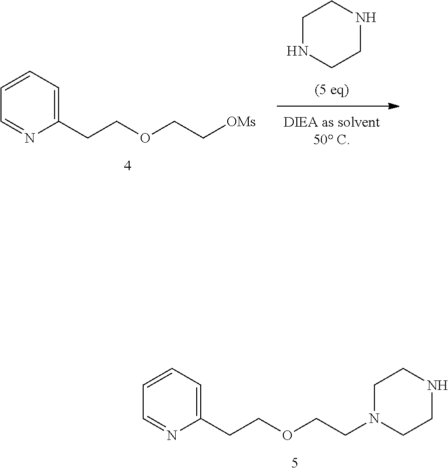 [SMILES: C1CNCCN1 ; CS(=O)(=O)OCCOCCc1ccccn1 ; c1ccc(CCOCCN2CCNCC2)nc1]

A solution of 2-(2-(pyridin-2-yl)ethoxy)ethyl methanesulfonate (360.0 mg, 1.47 mmol) and piperazine (632.1 mg, 7.35 mmol) in DIEA (8 mL) was heated to 50° C. and stirred for 18 h. TLC showed the reaction was completed. The solvent was concentrated under reduced pressure to give a crude product, which was purified by column to give 1-(2-(2-(pyridin-2-yl)ethoxy)ethyl)piperazine (270.0 mg, 78.2%) as an oil. LCMS (ESI) found: 236 [M+H]⁺.

### 5. Preparation of N-(3,4-dichloro-1H-indol-7-yl)-4-((4-(2-(2-(pyridin-2-yl)ethoxy)ethyl)piperazin-1-yl)sulfonyl)benzenesulfonamide

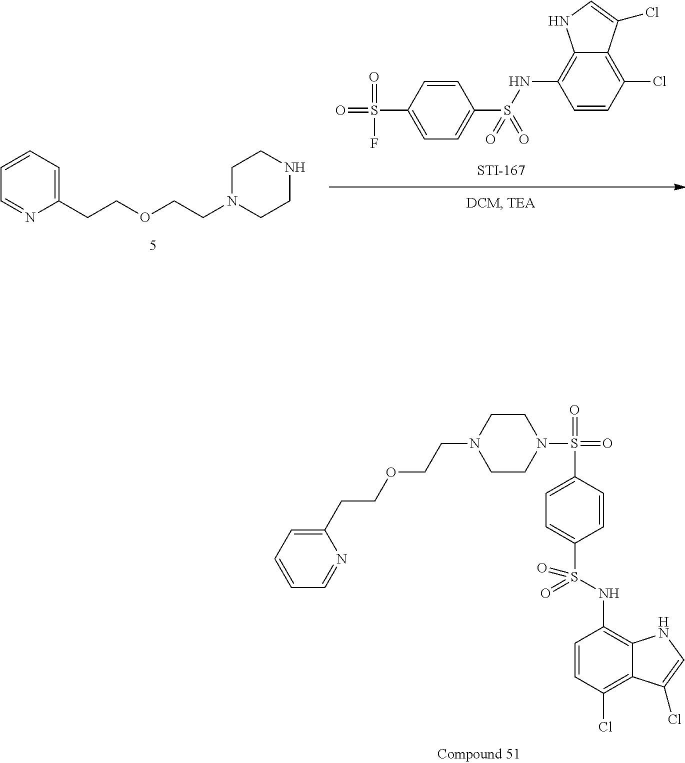 [SMILES: O=S(=O)(F)c1ccc(S(=O)(=O)Nc2ccc(Cl)c3c(Cl)c[nH]c23)cc1 ; c1ccc(CCOCCN2CCNCC2)nc1 ; O=S(=O)(Nc1ccc(Cl)c2c(Cl)c[nH]c12)c1ccc(S(=O)(=O)N2CCN(CCOCCc3ccccn3)CC2)cc1]

To a solution of 1-(2-(2-(pyridin-2-yl)ethoxy)ethyl)piperazine (20.0 mg, 0.085 mmol) and TEA (25.7 mg, 0.25 mmol) in dry DCM (2 mL) was added 4-(N-(3,4-dichloro-1H-indol-7-yl)sulfamoyl)benzenesulfonyl fluoride (36.0 mg, 0.085 mmol). The reaction mixture was stirred at rt for 18 h. LCMS showed the reaction was completed. The solvent was concentrated to give a crude product, which was purified by prep-TLC to give N-(3,4-dichloro-1H-indol-7-yl)-4-((4-(2-(2-(pyridin-2-yl)ethoxy)ethyl)piperazin-1-yl)sulfonyl)benzenesulfonamide (Compound 51, 32.6 mg, 60.1%) as a white solid. LCMS (ESI) found: 638 [M+H]⁺. ¹H NMR (400 MHz, DMSO-d6) δ 11.38 (d, J=2.3 Hz, 1H), 10.17 (s, 1H), 8.44-8.40 (m, 1H), 7.89-7.82 (m, 4H), 7.63-7.59 (m, 1H), 7.54 (d, J=2.7 Hz, 1H), 7.28 (d, J=7.8 Hz, 1H), 7.19-7.13 (m, 1H), 6.96 (d, J=8.1 Hz, 1H), 6.59 (d, J=8.1 Hz, 1H), 3.70 (t, J=6.6 Hz, 2H), 3.46 (t, J=5.5 Hz, 2H), 2.90 (t, J=6.6 Hz, 2H), 2.79 (s, 4H), 2.41 (s, 6H).

### Example 22: Preparation of 4-((1,4-diazepan-1-yl)sulfonyl)-N-(3,4-dichloro-1H-indol-7-yl)benzene sulfonamide; Compound 52

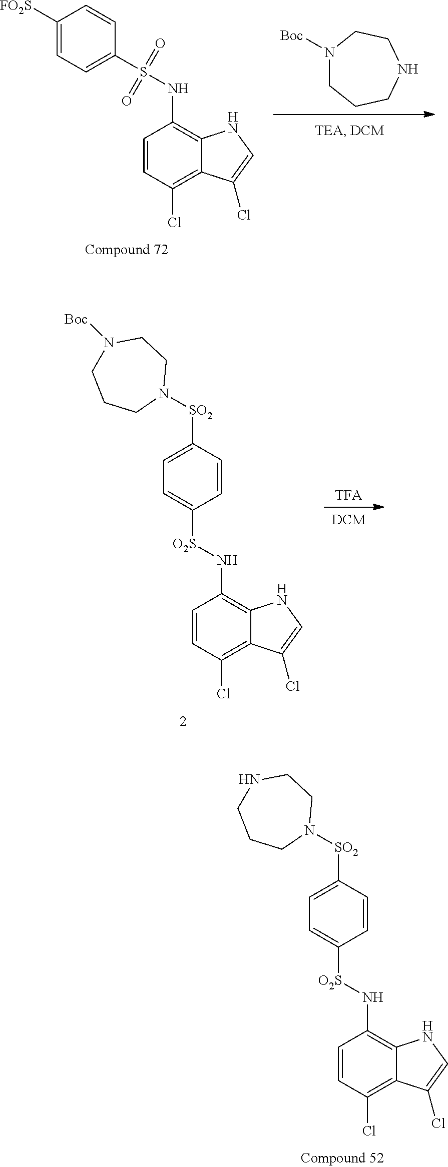

### 1. Preparation of tert-butyl 4-((4-(N-(3,4-dichloro-1H-indol-7-yl)sulfamoyl)phenyl)sulfonyl)-1,4-diazepane-1-carboxylate

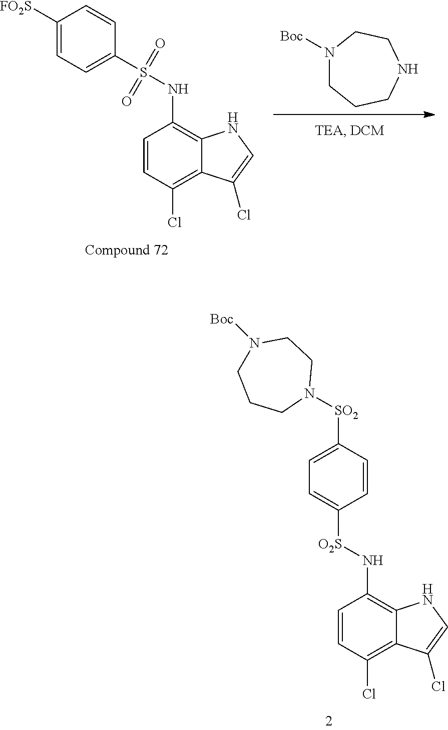

A solution of 4-(N-(3,4-dichloro-1H-indol-7-yl)sulfamoyl)benzenesulfonyl fluoride (60.0 mg, 0.15 mmol), TEA (45.4 mg, 0.45 mmol) and tert-butyl 1,4-diazepane-1-carboxylate (36.0 mg, 0.18 mmol) in DCM (2 mL) was stirred for 18 h at room temperature. The mixture was concentrated and purified by flash column to afford a crude product, which was further purified by column chromatography on silica gel eluting with PE/EA to give tert-butyl 4-((4-(N-(3,4-dichloro-1H-indol-7-yl)sulfamoyl)phenyl)sulfonyl)-1,4-diazepane-1-carboxylate (60.0 mg, yield: 66.3%) as a white solid. LCMS (ESI) found: 603 [M+H]⁺.

### 2. Preparation of 4-((1,4-diazepan-1-yl)sulfonyl)-N-(3,4-dichloro-1H-indol-7-yl)benzene sulfonamide

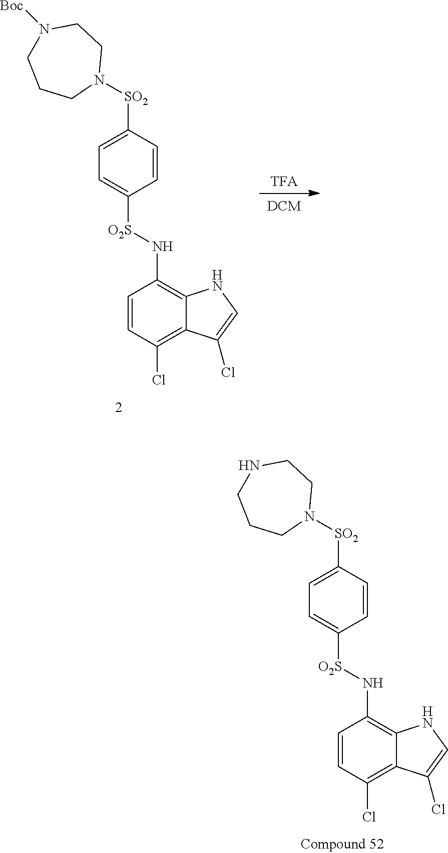

To a solution of tert-butyl 4-((4-(N-(3,4-dichloro-1H-indol-7-yl)sulfamoyl)phenyl)sulfonyl)-1,4-diazepane-1-carboxylate (30.0 mg, 0.06 mmol) in DCM (2 mL) was added TFA (0.5 mL). The mixture was stirred for 1 h at room temperature. The mixture was concentrated to give a crude product which was purified by prep-HPLC to afford N-(3,4-dichloro-1H-indol-7-yl)-4-((4-methylpiperazin-1-yl)sulfonyl) benzenesulfonamide (Compound 52, 7.3 mg, yield: 24.2%) as a white solid. LCMS (ESI) found: 503 [M+H]⁺. ¹H NMR (400 MHz, DMSO-d6) δ 11.31 (s, 1H), 7.92 (d, J=8.5 Hz, 2H), 7.82 (d, J=8.5 Hz, 2H), 7.27 (s, 1H), 6.73-6.67 (m, 2H), 3.29-3.22 (m, 4H), 3.09-2.98 (m, 4H), 1.88-1.78 (m, 2H).

### Example 23: Synthesis of N^{t}-(3,4-dichloro-1H-indol-7-yl)-N⁴-(2-(o-tolyloxy)benzyl)benzene-1,4-disulfonamide; Compound 56

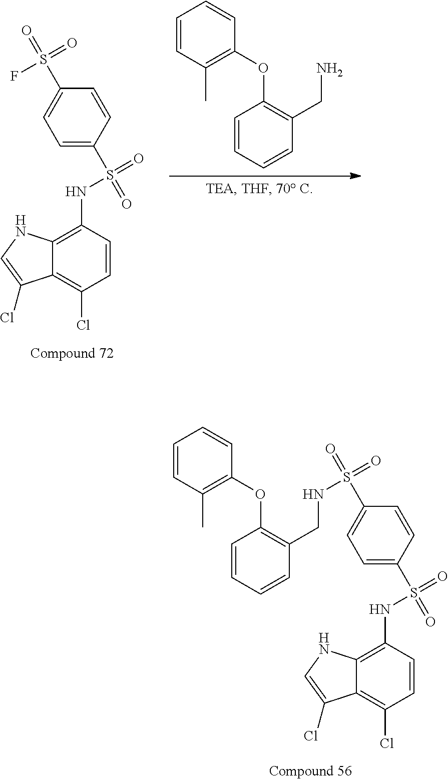

To a solution of 4-(N-(3,4-dichloro-1H-indol-7-yl)sulfamoyl)benzenesulfonyl fluoride (40 mg, 0.095 mmol) in anhydrous THF (5 mL) were added TEA (0.02 mL, 0.14 mmol) and (2-(o-tolyloxy)phenyl)methanamine (40 mg, 0.19 mmol). The resulting mixture was heated at 70° C. for 4 days. After completion, the reaction mixture was partitioned between DCM (15 mL) and water (15 mL). The two phases were separated, and the aqueous layer was extracted with DCM (10 mL) twice. The combined organic layers were washed brine, dried over Na₂SO₄, filtered and concentrated. The residue was purified by flash chromatography (silica gel, 0-50% ethyl acetate in petroleum ether), followed by prep-HPLC to afford N¹-(3,4-dichloro-1H-indol-7-yl)-N₄-(2-(o-tolyloxy)benzyl)benzene-1,4-disulfonamide (4 mg, 6.8%) as a white solid. LC/MS ESI (m/z): 614.2 (M−H)⁻. ¹HNMR (400 MHz, DMSO-d6) δ 11.47 (d, J=2.7 Hz, 1H), 10.21 (s, 1H), 8.38 (t, J=6.0 Hz, 1H), 7.88 (d, J=8.6 Hz, 2H), 7.83 (d, J=8.7 Hz, 2H), 7.55 (d, J=2.8 Hz, 1H), 7.32-7.25 (m, 2H), 7.17 (t, J=7.6 Hz, 1H), 7.13-7.06 (m, 2H), 6.94 (t, J=7.0 Hz, 1H), 6.88 (d, J=8.2 Hz, 1H), 6.73 (d, J=8.0 Hz, 1H), 6.58 (d, J=8.2 Hz, 1H), 6.46 (d, J=8.1 Hz, 1H), 4.09 (d, J=6.0 Hz, 2H), 2.07 (s, 3H).

### Example 24: Preparation of N-(3,4-dichloro-1H-indol-7-yl)-4-(piperidin-4-ylsulfonyl)benzene sulfonamide; Compound 57

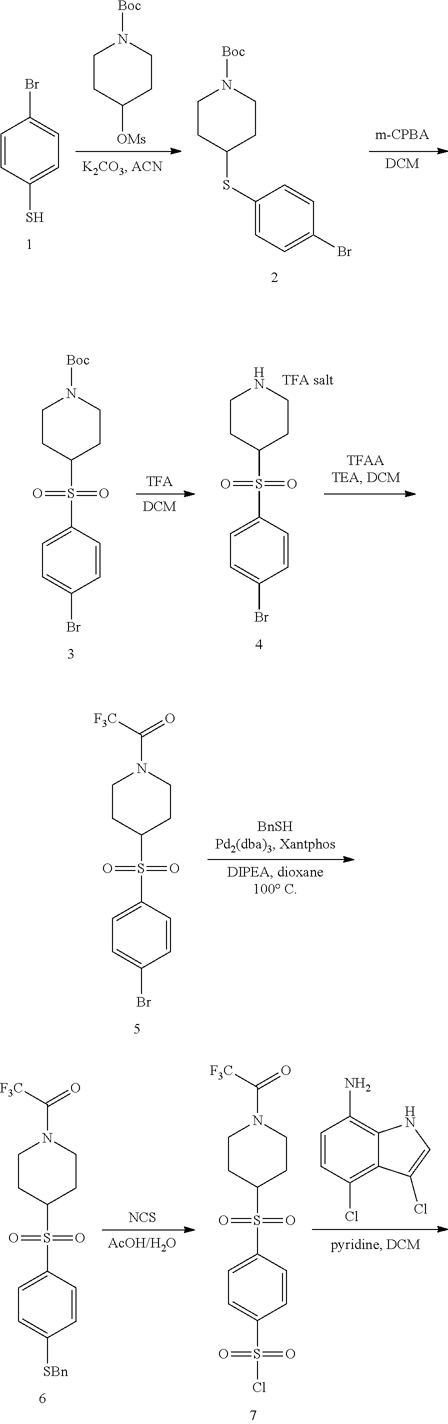

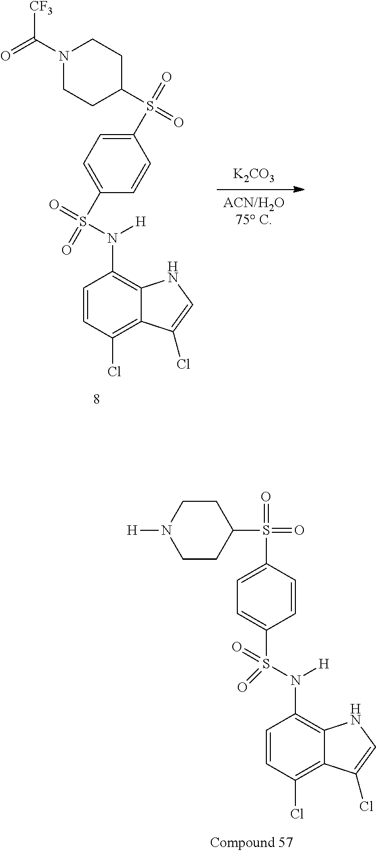

### 1. Preparation of tert-butyl 4-((4-bromophenyl)thio)piperidine-1-carboxylate

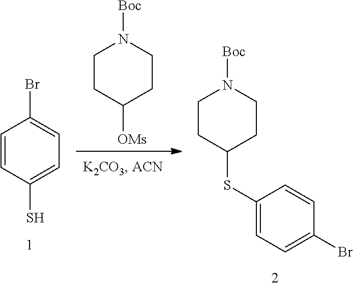 [SMILES: Sc1ccc(Br)cc1 ; CC(C)(C)OC(=O)N1CCC(OS(C)(=O)=O)CC1 ; CC(C)(C)OC(=O)N1CCC(Sc2ccc(Br)cc2)CC1]

To a solution of 4-bromobenzene-1-thiol (4.98 g, 26.44 mmol) in ACN (50 mL) were added tert-butyl 4-(methanesulfonyloxy)piperidine-1-carboxylate (7.39 g, 26.44 mmol) and K₂CO₃ (7.31 g, 52.89 mmol). The mixture was stirred at room temperature for 3 h. The solid was filtered and the filtrate was concentrated to get a residue, which was purified by flash column (EA/PE=0 to 20%) to afford tert-butyl 4-((4-bromophenyl)thio)piperidine-1-carboxylate (8.4 g, 85.3%) as a yellow oil.

### 2. Preparation of tert-butyl 4-((4-bromophenyl)sulfonyl)piperidine-1-carboxylate

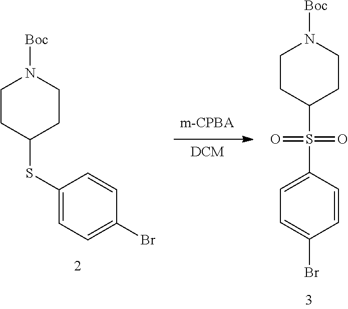

To a solution of tert-butyl 4-((4-bromophenyl)thio)piperidine-1-carboxylate (8.4 g, 22.56 mmol) in DCM (100 mL) was added m-CPBA (8.57 g, 49.63 mmol) at 0° C. The mixture was stirred at room temperature for 2 h. The mixture was then diluted with water (100 mL). The two phases were separated and the aqueous phase was washed with 10% aqueous Na₂SO₃ (50 mL) and then saturated NaHCO₃ (50 mL). The organic layer was dried over magnesium sulfate, filtered and concentrated to give tert-butyl 4-(4-bromobenzenesulfonyl)piperidine-1-carboxylate (7.0 g, 76.7%) as a white solid. LCMS (ESI) found: 404 [M+H]⁺.

### 3. Preparation of TFA salt of 4-((4-bromophenyl)sulfonyl)piperidine

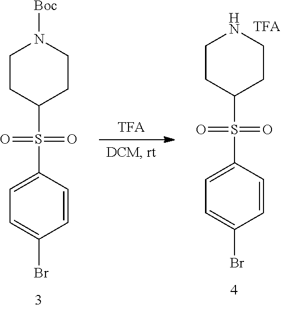 [SMILES: CC(C)(C)OC(=O)N1CCC(S(=O)(=O)c2ccc(Br)cc2)CC1 ; "DCM, rt" ; O=S(=O)(c1ccc(Br)cc1)C1CCNCC1 ; "TFA" ; "TFA"]

To a solution of tert-butyl 4-(4-bromobenzenesulfonyl)piperidine-1-carboxylate (3.5 g, 8.66 mmol) in DCM (20 mL) was added TFA (3.2 mL, 43.28 mmol). The mixture was stirred at room temperature for 1 h. Then the mixture was concentrated to get a residue, which was dissolved in DCM (20 mL). Then MeOH (30 mL) was added slowly. The solid formed was filtered off, washed with PE (10 mL) and dried under reduced pressure to afford 4-(4-bromobenzenesulfonyl)piperidine (TFA salt) (3.2 g, 92.4%) as white solid. LCMS (ESI) found: 304 [M+H]⁺.

### 4. Preparation of 1-(4-((4-bromophenyl)sulfonyl)piperidin-1-yl)-2,2,2-trifluoroethan-1-one

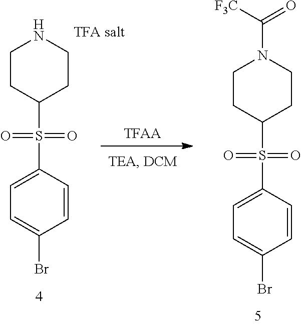

To a solution of TFA salt of 4-(4-bromobenzenesulfonyl)piperidine (2.6 g, 6.57 mmol) in DCM (10 mL) were added TFAA (1.52 g, 7.23 mmol) and TEA (1.99 g, 19.72 mmol). The mixture was stirred at room temperature for 1 h. Then the mixture was diluted with water (50 mL) and extracted with (50 mL×3). The combined organic layer was washed with brine (20 mL), dried over anhydrous Na₂SO₄, filtered and concentrated under reduced pressure to afford 1-(4-((4-bromophenyl)sulfonyl) piperidin-1-yl)-2,2,2-trifluoroethan-1-one (2.4 g, 91.2%) as a white solid. LCMS (ESI) found: 400 [M+H]⁺.

### 5. Preparation of 1-(4-((4-(benzylthio)phenyl)sulfonyl)piperidin-1-yl)-2,2,2-trifluoroethan-1-one

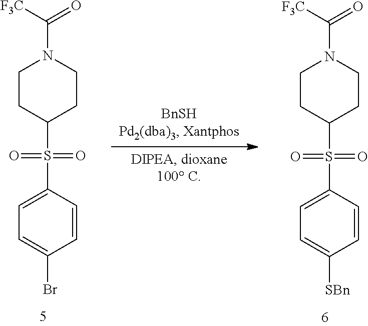

To a solution of 1-(4-((4-bromophenyl)sulfonyl)piperidin-1-yl)-2,2,2-trifluoroethan-1-one (2.4 g, 6.0 mmol) in dioxane (15 mL) were added BnSH (0.89 g, 7.20 mmol), Pd₂(dba)₃ (0.55 g, 0.60 mmol), Xantphos (0.35 g, 0.60 mmol) and DIPEA (1.82 g, 18.00 mmol). The mixture was charged with N₂ for three times and stirred at 100° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give 1-(4-((4-(benzylthio)phenyl)sulfonyl) piperidin-1-yl)-2,2,2-trifluoroethan-1-one (2.4 g, 90.2%) as a yellow solid. LCMS (ESI) found: 444 [M+H]⁺.

### 6. Preparation of 4-((1-(2,2,2-trifluoroacetyl)piperidin-4-yl)sulfonyl) benzenesulfonyl chloride

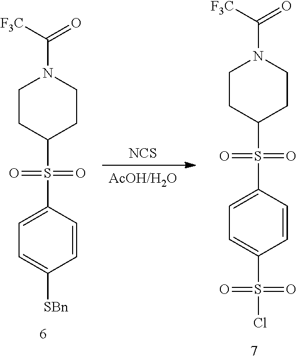

To a solution of 1-(4-((4-(benzylthio)phenyl)sulfonyl) piperidin-1-yl)-2,2,2-trifluoroethan-1-one (500 mg, 1.127 mmol) in DCM (5 mL) was added AcOH/H₂O (9/1, 5 mL). The mixture was stirred at room temperature for 2 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 4-((1-(2,2,2-trifluoroacetyl)piperidin-4-yl)sulfonyl) benzenesulfonyl chloride (410.0 mg, 86.6%) as a white solid. LCMS (ESI) found: 420 [M+H]⁺.

### 8. Preparation of N-(3,4-dichloro-1H-indol-7-yl)-4-((1-(2,2,2-trifluoroacetyl)piperidin-4-yl)sulfonyl)benzenesulfonamide

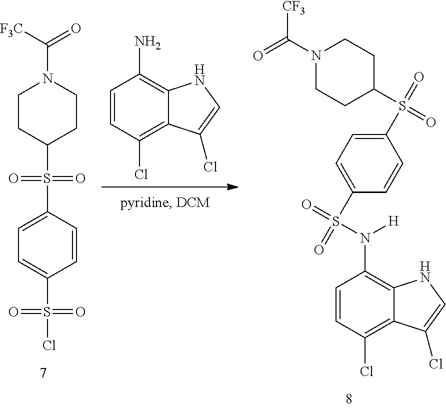

To a solution of 3,4-dichloro-1H-indol-7-amine (57.5 mg, 0.29 mmol) and pyridine (68.7 mg, 0.87 mmol) in DCM (5 mL) was added 4-((1-(2,2,2-trifluoroacetyl)piperidin-4-yl)sulfonyl) benzenesulfonyl chloride (120.0 mg, 0.29 mmol). The mixture was stirred at room temperature for 3 h. The mixture was concentrated to give a crude product, which was purified by flash column to afford N-(3,4-dichloro-1H-indol-7-yl)-4-((1-(2,2,2-trifluoroacetyl) piperidin-4-yl)sulfonyl)benzenesulfonamide (150.0 mg, 89.8%) as a white solid. LCMS (ESI) found: 584 [M+H]⁺.

### 9. Preparation of N-(3,4-dichloro-1H-indol-7-yl)-4-(piperidin-4-ylsulfonyl) benzenesulfonamide

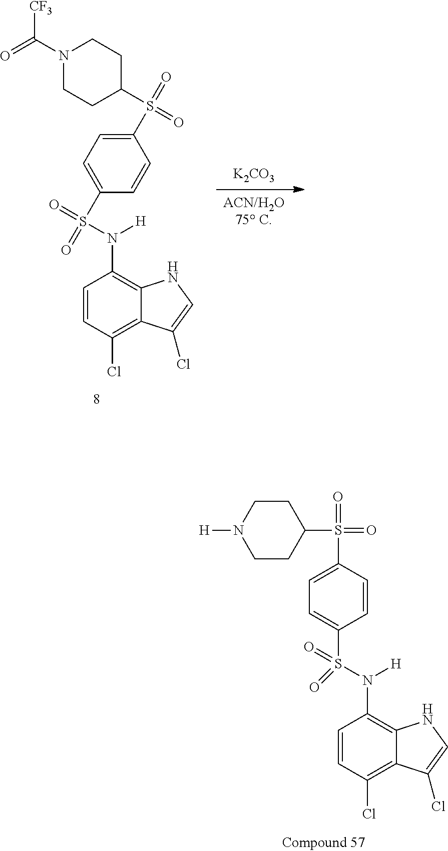

To a solution of N-(3,4-dichloro-1H-indol-7-yl)-4-((1-(2,2,2-trifluoroacetyl)piperidin-4-yl)sulfonyl)benzenesulfonamide (150.0 mg, 0.26 mmol) in ACN/H₂O (4/1, 5 mL) was added K₂CO₃ (70.8 mg, 0.51 mmol). The mixture was stirred at 75° C. overnight. Then the mixture was cooled down to room temperature, filtered and concentrated to give a crude product, which was purified by prep-HPLC to afford N-(3,4-dichloro-1H-indol-7-yl)-4-(piperidin-4-yl sulfonyl)benzenesulfonamide (HCOOH salt) (Compound 57, 120.0 mg, 95.7%). LCMS (ESI) found: 490 [M+H]⁺. ¹H NMR (400 MHz, CD₃OD) δ 8.50 (s, 0.66H), 8.05-7.88 (m, 4H), 7.29 (s, 1H), 6.85 (d, J=8.1 Hz, 1H), 6.54 (d, J=8.1 Hz, 1H), 3.56-3.35 (m, 3H), 2.92-2.83 (m, 2H), 2.08-2.00 (m, 2H), 1.89-1.78 (m, 2H).

### Example 25: Preparation of N-(3,4-dichloro-1H-indol-7-yl)-4-((1-methylpiperidin-4-yl)sulfonyl)benzene sulfonamide; Compound 58

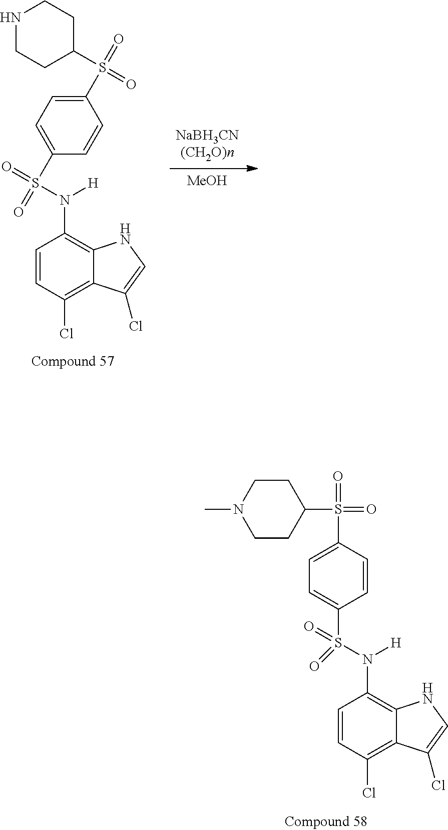 [SMILES: "MeOH" ; O=S(=O)(Nc1ccc(Cl)c2c(Cl)c[nH]c12)c1ccc(S(=O)(=O)C2CCNCC2)cc1 ; "NaBH3CN(CH2O)n" ; CN1CCC(S(=O)(=O)c2ccc(S(=O)(=O)Nc3ccc(Cl)c4c(Cl)c[nH]c34)cc2)CC1]

### Preparation of N-(3,4-dichloro-1H-indol-7-yl)-4-((1-methylpiperidin-4-yl)sulfonyl) benzenesulfonamide

To a solution of N-(3,4-dichloro-1H-indol-7-yl)-4-(piperidin-4-ylsulfonyl)benzenesulfonamide (100.0 mg, 0.21 mmol) in MeOH (10 mL) were added (CH₂O)n (12.6 mg, 0.42 mmol), a few drops of AcOH and NaBH₃CN (19.3 mg, 0.31 mmol). The mixture was stirred at room temperature overnight. Then the mixture was concentrated to give a crude product, which was purified by prep-HPLC to afford N-(3,4-dichloro-1H-indol-7-yl)-4-((1-methylpiperidin-4-yl) sulfonyl)benzene sulfonamide (Compound 58, 58.0 mg, 53.5%) as a white solid. LCMS (ESI) found: 502 [M+H]⁺. ¹H NMR (400 MHz, DMSO-d6) δ 11.42 (d, J=2.6 Hz, 1H), 9.74 (s, 1H), 7.91 (dd, J=23.8, 8.6 Hz, 4H), 7.48 (d, J=2.8 Hz, 1H), 6.86 (d, J=8.1 Hz, 1H), 6.48 (d, J=8.2 Hz, 1H), 3.50 (t, J=12.0 Hz, 1H), 3.34 (d, J=11.5 Hz, 2H), 2.76-2.71 (m, 2H), 2.61 (s, 3H), 1.94-1.90 (m, 2H), 1.70-1.54 (m, 2H).

### Example 26: Preparation of N-(3,4-dichloro-1H-indol-7-yl)-4-(1-(piperazin-1-yl)cyclopropyl) benzenesulfonamide Compound 59

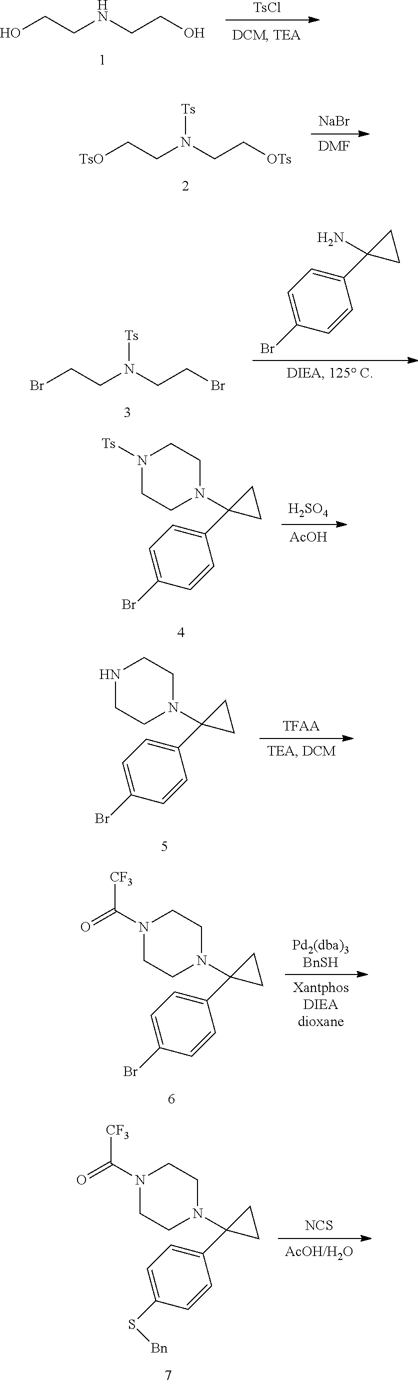

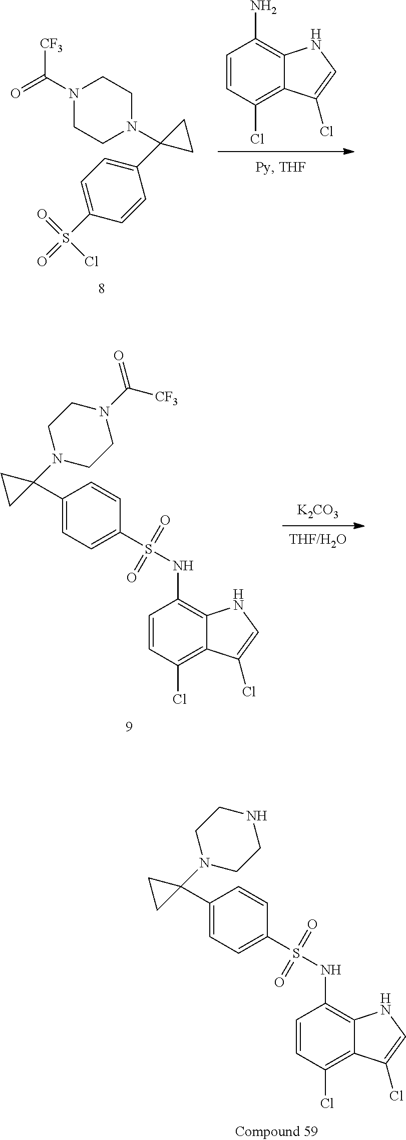 [SMILES: O=C(N1CCN(C2(c3ccc(S(=O)(=O)Cl)cc3)CC2)CC1)C(F)(F)F ; Nc1ccc(Cl)c2c(Cl)c[nH]c12 ; O=C(N1CCN(C2(c3ccc(S(=O)(=O)Nc4ccc(Cl)c5c(Cl)c[nH]c45)cc3)CC2)CC1)C(F)(F)F ; "K2CO3" ; "THF/H2O" ; O=S(=O)(Nc1ccc(Cl)c2c(Cl)c[nH]c12)c1ccc(C2(N3CCNCC3)CC2)cc1]

### 1. Preparation of (tosylazanediyl)bis(ethane-2,1-diyl) bis(4-methylbenzene sulfonate)

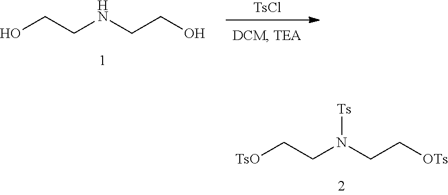 [SMILES: OCCNCCO ; "TsCl" ; Cc1ccc(S(=O)(=O)OCCN(CCOS(=O)(=O)c2ccc(C)cc2)S(=O)(=O)c2ccc(C)cc2)cc1]

To a solution of 2,2′-azanediylbis(ethan-1-ol) (2.4 g, 22.83 mmol) in DCM (150 mL) were added TEA (12.7 mL, 91.31 mmol) and TsCl (15.2 g, 79.89 mmol). The mixture was stirred at rt overnight. The mixture was concentrated to give a crude product, which was purified by column chromatography on silica gel eluting with PE/EA from 10/1 to 1/1 to give (tosylazanediyl)bis(ethane-2,1-diyl) bis(4-methylbenzene sulfonate) (12.6 g, 22.19 mmol, 97.23%) as a white solid. LCMS (ESI) found: 568 [M+H]⁺.

### 2. Preparation of N,N-bis(2-bromoethyl)-4-methylbenzenesulfonamide

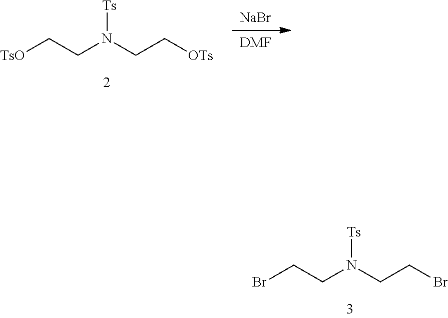

To a solution of (tosylazanediyl)bis(ethane-2,1-diyl) bis(4-methylbenzene sulfonate) (1.0 g, 1.76 mmol) in anhydrous DMF (30 mL) was added sodium bromide (907.4 mg, 8.81 mmol). The mixture was stirred at 120° C. overnight. TLC (PE/EA=10/1) showed a new spot was detected. The mixture was concentrate to give a crude product, which was purified by flash column eluting with PE/EA from 98/2 to 80/20 to give N,N-bis(2-bromoethyl)-4-methylbenzenesulfonamide (410.0 mg, 60.4%) as a colorless oil. LCMS (ESI) found: 384 [M+H]⁺.

### 3. Preparation of 1-(1-(4-bromophenyl)cyclopropyl)-4-tosylpiperazine

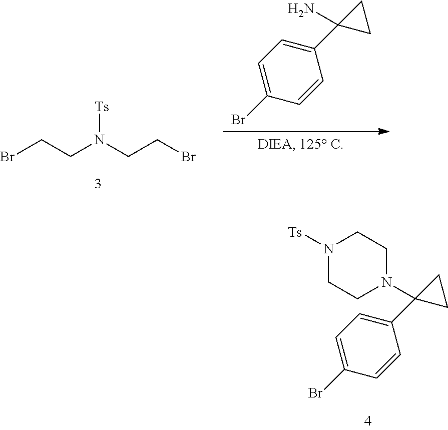

A mixture of N,N-bis(2-bromoethyl)-4-methylbenzenesulfonamide (243.4 mg, 1.15 mmol) and 1-(4-bromophenyl)cyclopropan-1-amine (340.0 mg, 0.88 mmol) in DIPEA (4 mL) was stirred at 120° C. in a sealed tube overnight. LCMS showed the desired mass was detected. The mixture was cooled down to room temperature and diluted with water (100 mL). The aqueous phase was extracted with DCM (100 mL×3). The combined organic layer was concentrated to give a crude product, which was purified by flash column eluting with PE/EA from 98/2 to 50/50 to give 1-(1-(4-bromophenyl)cyclopropyl)-4-tosylpiperazine (310.0 mg, 80.6%) as a yellow solid. LCMS (ESI) found: 435 [M+H]⁺.

### 4. Preparation of 1-(1-(4-bromophenyl)cyclopropyl)piperazine

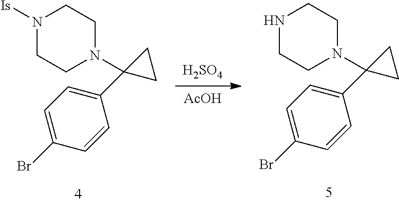

To a solution of 1-(1-(4-bromophenyl)cyclopropyl)-4-tosylpiperazine (310.0 mg, 0.71 mmol) in AcOH (10 mL) was added sulfuric acid (4 mL, 35.60 mmol). The mixture was stirred at 100° C. overnight. The mixture was concentrated and basified with 20% aqueous NaOH to pH=8. The mixture was extracted with DCM (30 mL×2). The combined organic layer was concentrated to give a crude product, which was used directed for the next step. LCMS (ESI) found: 281 [M+H]⁺.

### 5. Preparation of 1-(4-(1-(4-bromophenyl)cyclopropyl)piperazin-1-yl)-2,2,2-trifluoroethan-1-one

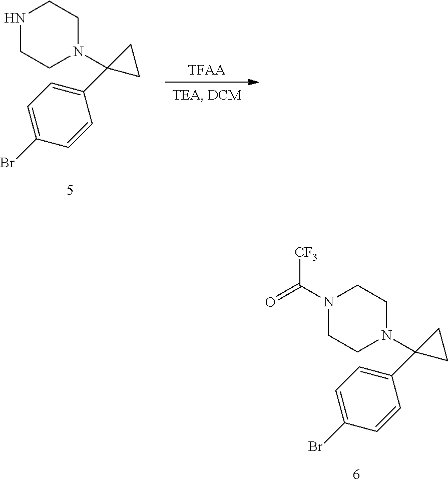 [SMILES: Brc1ccc(C2(N3CCNCC3)CC2)cc1 ; "TFAA" ; "TEA, DCM" ; O=C(N1CCN(C2(c3ccc(Br)cc3)CC2)CC1)C(F)(F)F]

To a solution of 1-(1-(4-bromophenyl)cyclopropyl)piperazine (199.6 mg, 0.71 mmol) in DCM (2 mL) were added TEA (0.2 mL, 1.420 mmol) and TFAA (223.6 mg, 1.06 mmol). The mixture was stirred at rt for 3 h. The mixture was diluted with water (20 mL). The mixture was extracted with DCM (20 mL×3). The combined organic layer was concentrated to give a crude product, which was purified by flash column eluting with PE/EA from 95/5 to 30/70 to give 1-(4-(1-(4-bromophenyl)cyclopropyl)piperazin-1-yl)-2,2,2-trifluoroethan-1-one (127.0 mg, 47.4%) as a yellow solid. LCMS (ESI) found: 377 [M+H]⁺.

### 6. Preparation of 1-(4-(1-(4-(benzylthio)phenyl)cyclopropyl)piperazin-1-yl)-2,2,2-trifluoroethan-1-one

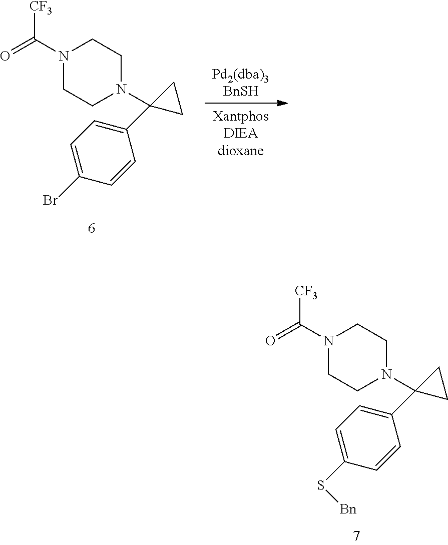 [SMILES: O=C(N1CCN(C2(c3ccc(Br)cc3)CC2)CC1)C(F)(F)F ; "Pd2(dba)3BnSH" ; "XantphosDIEAdioxane" ; O=C(N1CCN(C2(c3ccc(SCc4ccccc4)cc3)CC2)CC1)C(F)(F)F]

To a solution of 1-(4-(1-(4-bromophenyl)cyclopropyl)piperazin-1-yl)-2,2,2-trifluoroethan-1-one (525.0 mg, 1.392 mmol), DIPEA (538.6 mg, 4.18 mmol) in dioxane (2 mL) were added BnSH (345.2 mg, 2.78 mmol), Pd₂(dba)₃ (191.2 mg, 0.21 mmol) and Xantphos (362.4 mg, 0.63 mmol). The mixture was charged with N₂ for three times and stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 20/1 to 5/1 to give 1-(4-(1-(4-(benzylthio)phenyl)cyclopropyl)piperazin-1-yl)-2,2,2-trifluoroethan-1-one (540.0 mg, 92.3%) as a yellow solid. LCMS (ESI) found: 421 [M+H]⁺.

### 7. Preparation of 4-(1-(4-(2,2,2-trifluoroacetyl)piperazin-1-yl)cyclopropyl)benzene sulfonyl chloride

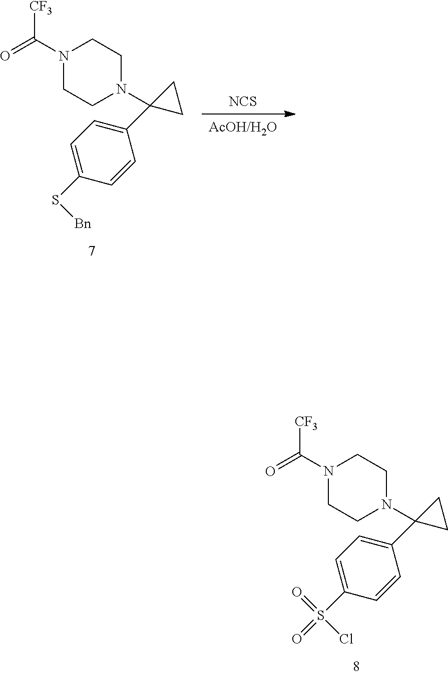 [SMILES: O=C(N1CCN(C2(c3ccc(SCc4ccccc4)cc3)CC2)CC1)C(F)(F)F ; "AcOH/H2O" ; "NCS" ; O=C(N1CCN(C2(c3ccc(S(=O)(=O)Cl)cc3)CC2)CC1)C(F)(F)F]

To a solution of 1-(4-(1-(4-(benzylthio)phenyl)cyclopropyl)piperazin-1-yl)-2,2,2-trifluoroethan-1-one (100.0 mg, 0.24 mmol) in AcOH/H₂O (9/1, 3 mL) was added NCS (133.4 mg, 1.0 mmol). The mixture was stirred at rt for 2 h. Then the reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 4-(1-(4-(2,2,2-trifluoroacetyl)piperazin-1-yl)cyclopropyl)benzenesulfonyl chloride (76.0 mg, 80.5%) as a white solid. LCMS (ESI) found: 397 [M+H]⁺.

### 8. Preparation of N-(3,4-dichloro-1H-indol-7-yl)-4-(1-(4-(2,2,2-trifluoroacetyl)piperazin-1-yl)cyclopropyl)benzenesulfonamide

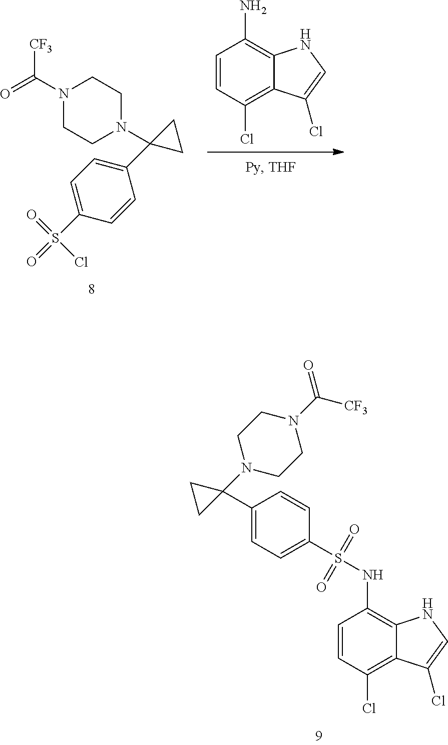 [SMILES: O=C(N1CCN(C2(c3ccc(S(=O)(=O)Cl)cc3)CC2)CC1)C(F)(F)F ; Nc1ccc(Cl)c2c(Cl)c[nH]c12 ; O=C(N1CCN(C2(c3ccc(S(=O)(=O)Nc4ccc(Cl)c5c(Cl)c[nH]c45)cc3)CC2)CC1)C(F)(F)F]

To a solution of 3,4-dichloro-1H-indol-7-amine (38.5 mg, 0.19 mmol) and pyridine (0.02 mL, 0.19 mmol) in THE (2 mL) was added 4-(1-(4-(2,2,2-trifluoroacetyl)piperazin-1-yl)cyclopropyl)benzene sulfonyl chloride (76.0 mg, 0.19 mmol). The mixture was stirred at rt for 4 h. Then the mixture was concentrated to give a crude product, which was purified by column chromatography on silica gel eluting with PE/EA from 15/1 to 5/1 to afford N-(3,4-dichloro-1H-indol-7-yl)-4-(1-(4-(2,2,2-trifluoroacetyl)piperazin-1-yl)cyclopropyl) benzenesulfonamide (82.0 mg, 76.3%) as a white solid. LCMS (ESI) found: 561 [M+H]⁺.

### 9. Preparation of N-(3,4-dichloro-1H-indol-7-yl)-4-(1-(piperazin-1-yl)cyclopropyl) benzenesulfonamide

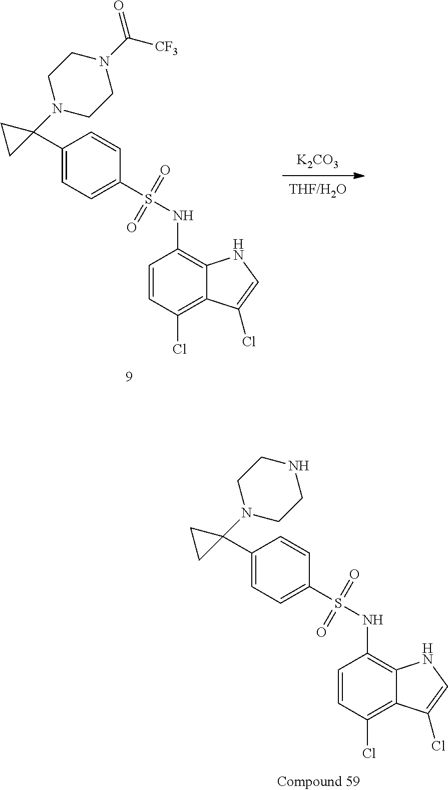 [SMILES: O=C(N1CCN(C2(c3ccc(S(=O)(=O)Nc4ccc(Cl)c5c(Cl)c[nH]c45)cc3)CC2)CC1)C(F)(F)F ; "K2CO3" ; "THF/H2O" ; O=S(=O)(Nc1ccc(Cl)c2c(Cl)c[nH]c12)c1ccc(C2(N3CCNCC3)CC2)cc1]

To a solution of N-(3,4-dichloro-1H-indol-7-yl)-4-(1-(4-(2,2,2-trifluoroacetyl)piperazin-1-yl)cyclopropyl)benzenesulfonamide (43.0 mg, 0.08 mmol) in THF/H₂O (2 mL) was added K₂CO₃ (31.8 mg, 0.23 mmol). The mixture was stirred at 50° C. overnight. The mixture was concentrated to give a crude product, which was purified by reversed phase flash eluting with MeOH/H₂O from 5/95 to 95/5 to give N-(3,4-dichloro-1H-indol-7-yl)-4-(1-(piperazin-1-yl)cyclopropyl)benzenesulfonamide (Compound 59, 19.0 mg, 53.3%) as a white solid. LCMS (ESI) found: 465 [M+H]⁻. ¹H NMR (400 MHz, DMSO-d6) δ 7.69 (d, J=7.6 Hz, 2H), 7.20 (d, J=7.9 Hz, 2H), 7.14 (s, 1H), 6.75 (d, J=8.3 Hz, 1H), 6.60 (d, J=8.0 Hz, 1H), 2.65 (s, 4H), 2.37 (s, 4H), 0.81 (s, 2H), 0.70 (s, 2H).

### Example 27: Preparation of N-(3,4-dichloro-1H-indol-7-yl)-4-(1-(piperazin-1-yl)cyclopropyl) benzenesulfonamide; Compound 60

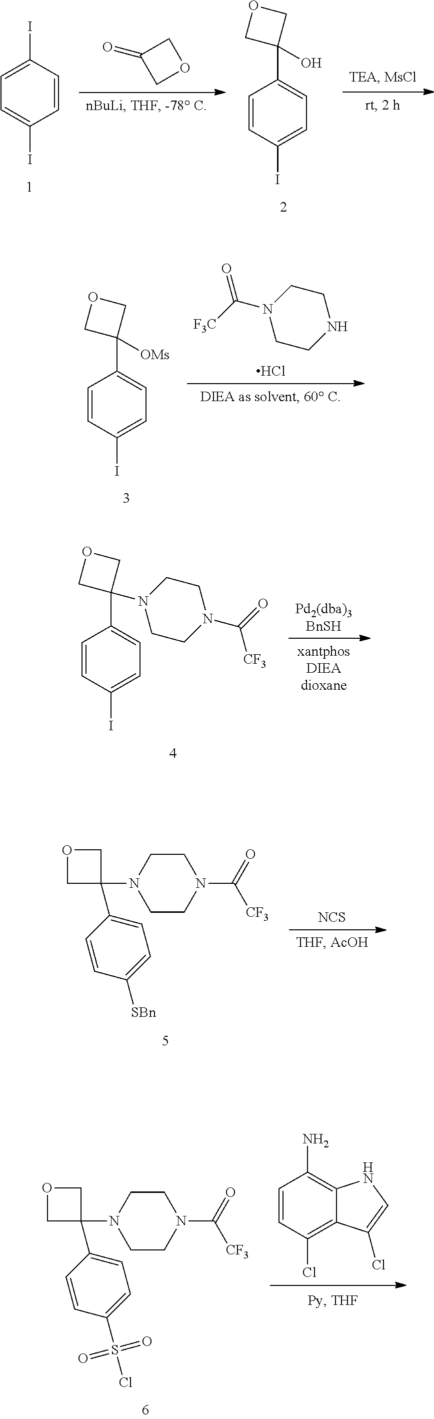

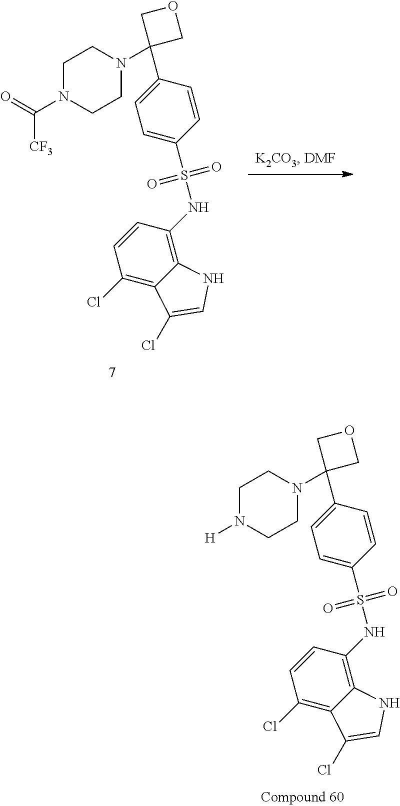 [SMILES: O=C(N1CCN(C2(c3ccc(S(=O)(=O)Nc4ccc(Cl)c5c(Cl)c[nH]c45)cc3)COC2)CC1)C(F)(F)F ; O=S(=O)(Nc1ccc(Cl)c2c(Cl)c[nH]c12)c1ccc(C2(N3CCNCC3)COC2)cc1]

### 1. Preparation of 3-(4-iodophenyl)oxetan-3-ol

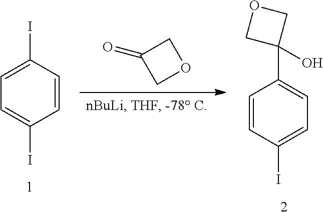

To a solution of 1,4-diiodobenzene (15.0 g, 45.47 mmol) in THF (130 mL) was added n-BuLi (18.2 mL, 36.37 mmol) at −78° C. The mixture was stirred at −78° C. for 30 min. Then thenoxetan-3-one (2.9 g, 40.92 mmol) in THF (20 mL) was added. The reaction mixture was allowed to warm to rt and further stirred for another 1 h at rt. The reaction mixture was treated with sat. NH₄Cl (100 mL) and extracted with EtOAc (200 mL×3). The combined organic layer was washed with brine, dried over Na₂SO₄, filtered and concentrated under reduced pressure to give a crude product, which was purified by column chromatography on silica gel to afford 3-(4-iodophenyl)oxetan-3-ol (6.2 g, 49.4%) as a white solid. LCMS (ESI) found: 277 [M+H]⁺.

### 2. Preparation of 3-(4-iodophenyl)oxetan-3-yl methanesulfonate

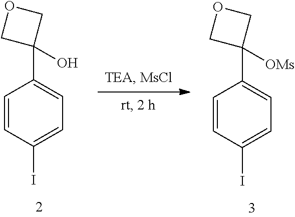 [SMILES: OC1(c2ccc(I)cc2)COC1 ; "TEA, MsCl" ; "rt, 2 h" ; CS(=O)(=O)OC1(c2ccc(I)cc2)COC1]

To a solution of 3-(4-iodophenyl)oxetan-3-ol (1.2 g, 4.35 mmol) in DCM (3 mL) were added MsCl (991.0 mg, 8.69 mmol) and TEA (1.1 g, 10.87 mmol). The mixture was stirred at 0° C. for 2 h. After completion, the mixture was diluted with brine and extracted with DCM (5 mL×3). The combined organic phase was concentrated to give a crude product, which was used directly for the next step. LCMS (ESI) found: 355 [M+H]⁺.

### 3. Preparation of 2,2,2-trifluoro-1-(4-(3-(4-iodophenyl)oxetan-3-yl)piperazin-1-yl)ethan-1-one

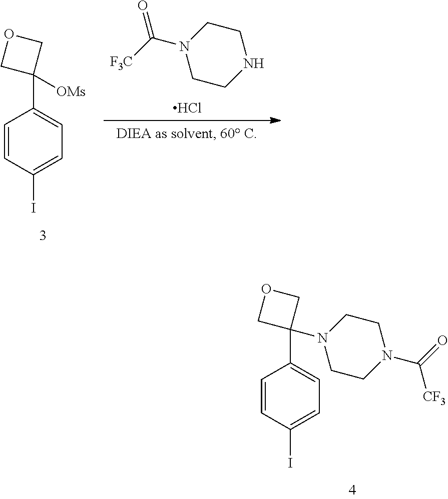

To the crude 3-(4-iodophenyl)oxetan-3-yl methanesulfonate (1.5 g, 4.34 mmol) in DIPEA (15 mL) was added 2,2,2-trifluoro-1-(piperazin-1-yl)ethan-1-one hydrochloride (1.1 g, 5.21 mmol). The mixture was stirred at 60° C. overnight. The mixture was concentrated to get a residue, which was purified by flash column to give a crude product, which was purified again by reversed phase combi-flash to give 2,2,2-trifluoro-1-(4-(3-(4-iodophenyl)oxetan-3-yl)piperazin-1-yl)ethan-1-one (160.0 mg, 8.4%) as a yellow solid. LCMS (ESI) found: 441 [M+H]⁺.

### 4. Preparation of 1-(4-(3-(4-(benzylthio)phenyl)oxetan-3-yl)piperazin-1-yl)-2,2,2-trifluoroethan-1-one

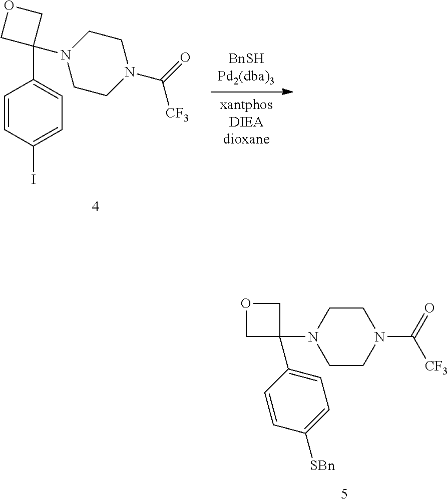

To a mixture of 2,2,2-trifluoro-1-(4-(3-(4-iodophenyl)oxetan-3-yl)piperazin-1-yl)ethan-1-one (150.0 mg, 0.34 mmol), Pd₂dba₃ (31.2 mg, 0.03 mmol) and Xantphos (39.4 mg, 0.07 mmol) in dioxane (2 mL) were added DIPEA (87.9 mg, 0.68 mmol) and BnSH (59.2 mg, 0.48 mmol). The mixture was charged with N₂ for three times and stirred at 110° C. overnight. The mixture was cooled to rt and concentrated to give a crude product, which was purified by flash column to give 1-(4-(3-(4-(benzylthio)phenyl)oxetan-3-yl)piperazin-1-yl)-2,2,2-trifluoroethan-1-one (120.0 mg, 80.7%) as a yellow solid. LCMS (ESI) found: 437 [M+H]⁺.

### 5. Preparation of 4-(3-(4-(2,2,2-trifluoroacetyl)piperazin-1-yl)oxetan-3-yl)benzene sulfonyl chloride

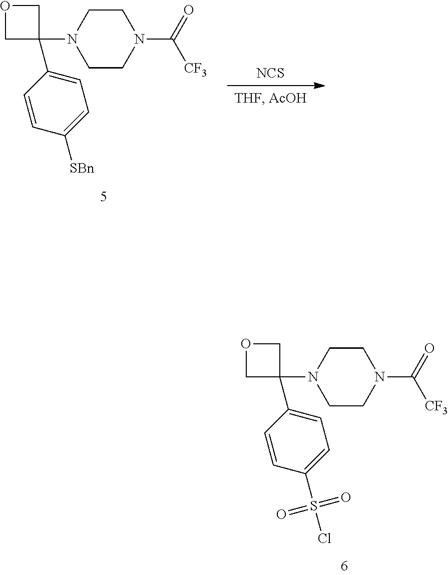

To a solution of 1-(4-(3-(4-(benzylthio)phenyl)oxetan-3-yl)piperazin-1-yl)-2,2,2-trifluoroethan-1-one (110.0 mg, 0.25 mmol) in AcOH/H₂O (9/1, 1 mL) was added NCS (141.8 mg, 1.06 mmol). The mixture was stirred at rt for 3 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 4-(3-(4-(2,2,2-trifluoroacetyl)piperazin-1-yl)oxetan-3-yl)benzenesulfonyl chloride (78.0 mg, 75.0%) as a white solid. LCMS (ESI) found: 413 [M+H]⁺.

### 6. Preparation of N-(3,4-dichloro-1H-indol-7-yl)-4-(3-(4-(2,2,2-trifluoroacetyl) piperazin-1-yl)oxetan-3-yl)benzenesulfonamide

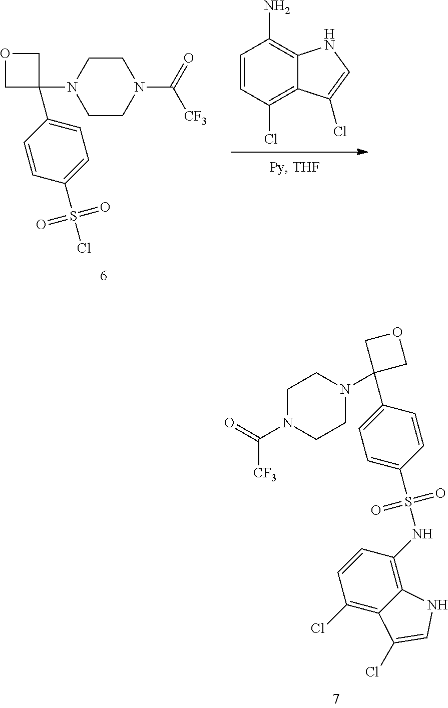 [SMILES: O=C(N1CCN(C2(c3ccc(S(=O)(=O)Cl)cc3)COC2)CC1)C(F)(F)F ; Nc1ccc(Cl)c2c(Cl)c[nH]c12 ; O=C(N1CCN(C2(c3ccc(S(=O)(=O)Nc4ccc(Cl)c5c(Cl)c[nH]c45)cc3)COC2)CC1)C(F)(F)F]

To a solution of 3,4-dichloro-1H-indol-7-amine (38.0 mg, 0.19 mmol) and pyridine (44.8 mg, 0.57 mmol) in THF (1 mL) was added 4-(3-(4-(2,2,2-trifluoroacetyl)piperazin-1-yl)oxetan-3-yl)benzenesulfonyl chloride (78.0 mg, 0.19 mmol). The mixture was stirred at rt overnight. The mixture was concentrated to give a crude product, which was purified by prep-TLC eluted with PE/EA=1/1 to give N-(3,4-dichloro-1H-indol-7-yl)-4-(3-(4-(2,2,2-trifluoroacetyl) piperazin-1-yl)oxetan-3-yl)benzenesulfonamide (102.0 mg, 93.5%) as a white solid. LCMS (ESI) found: 577 [M+H]⁺.

### 7. Preparation of N-(3,4-dichloro-1H-indol-7-yl)-4-(3-(piperazin-1-yl)oxetan-3-yl)benzenesulfonamide

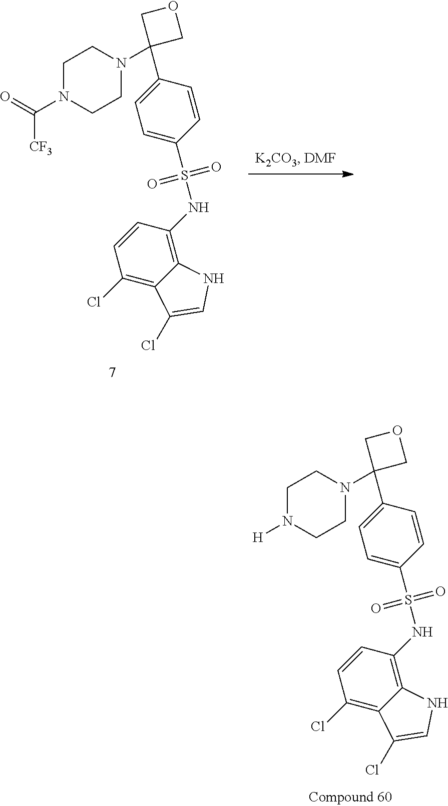 [SMILES: O=C(N1CCN(C2(c3ccc(S(=O)(=O)Nc4ccc(Cl)c5c(Cl)c[nH]c45)cc3)COC2)CC1)C(F)(F)F ; O=S(=O)(Nc1ccc(Cl)c2c(Cl)c[nH]c12)c1ccc(C2(N3CCNCC3)COC2)cc1]

To a solution of N-(3,4-dichloro-1H-indol-7-yl)-4-(3-(4-(2,2,2-trifluoroacetyl)piperazin-1-yl)oxetan-3-yl)benzenesulfonamide (85.0 mg, 0.15 mmol) in THF/H₂O (4/1, 2 mL) was added K₂CO₃ (61.0 mg, 0.44 mmol). The mixture was stirred at 50° C. for 4 h. The mixture was filtered and the organic layer was concentrated to give a crude product, which was purified by flash to give about 90.0 mg crude product, 30.0 mg of which was re-purified by prep-TLC to give N-(3,4-dichloro-1H-indol-7-yl)-4-(3-(piperazin-1-yl)oxetan-3-yl)benzene sulfonamide (Compound 60, 18.0 mg, 25.4%) as a white solid. LCMS (ESI) found: 481 [M+H]⁺. ¹H NMR (400 MHz, DMSO-d6) δ 11.62 (s, 1H), 8.97 (s, 1H), 7.79 (d, J=8.4 Hz, 2H), 7.53 (s, 1H), 7.31 (d, J=8.3 Hz, 2H), 6.95 (d, J=8.2 Hz, 1H), 6.75 (d, J=8.2 Hz, 1H), 4.82-4.65 (m, 4H), 3.07-3.04 (m, 4H), 2.32 (s, 4H).

### Example 28: Preparation of N-(3,4-dichloro-1H-indol-7-yl)-4-(1-methyl-1H-pyrazol-4-yl)benzenesulfonamide Compound 61

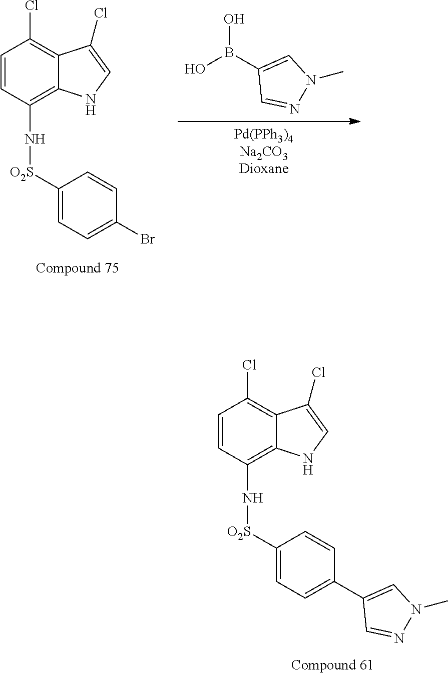

### Preparation of N-(3,4-dichloro-1H-indol-7-yl)-4-(1-methyl-1H-pyrazol-4-yl)benzenesulfonamide

To a solution of 4-bromo-N-(3,4-dichloro-1H-indol-7-yl)benzenesulfonamide (80.0 mg, 0.19 mmol) and (1-methyl-1H-pyrazol-4-yl)boronic acid (47.96 mg, 0.38 mmol) in dioxane (3 mL) were added Pd(PPh₃)₄ (22.0 mg, 0.019 mmol) and Na₂CO₃ (60.4 mg, 0.57 mmol). The mixture was evaporated and backfilled with N₂ for three times. The mixture was stirred at 110° C. under N₂ atmosphere overnight until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by prep-HPLC to give N-(3,4-dichloro-1H-indol-7-yl)-4-(1-methyl-1H-pyrazol-4-yl)benzene sulfonamide (Compound 61, 2.0 mg, 2.5%) to get the product as a white solid. LCMS (ESI): 421 [M+H]⁺. ¹HNMR (400 MHz, DMSO-d6) δ 11.40 (s, 1H), 10.07 (s, 1H), 8.23 (s, 1H), 7.93 (s, 1H), 7.67 (s, 4H), 7.51 (s, 1H), 6.91 (d, J=8.2 Hz, 1H), 6.71 (d, J=8.2 Hz, 1H), 3.86 (s, 3H).

### Example 29: Preparation of N-(4-(N-(3,4-dichloro-1H-indol-7-yl)sulfamoyl)phenyl)-1-methyl-N-propylpiperidine-4-sulfonamide; Compound 62

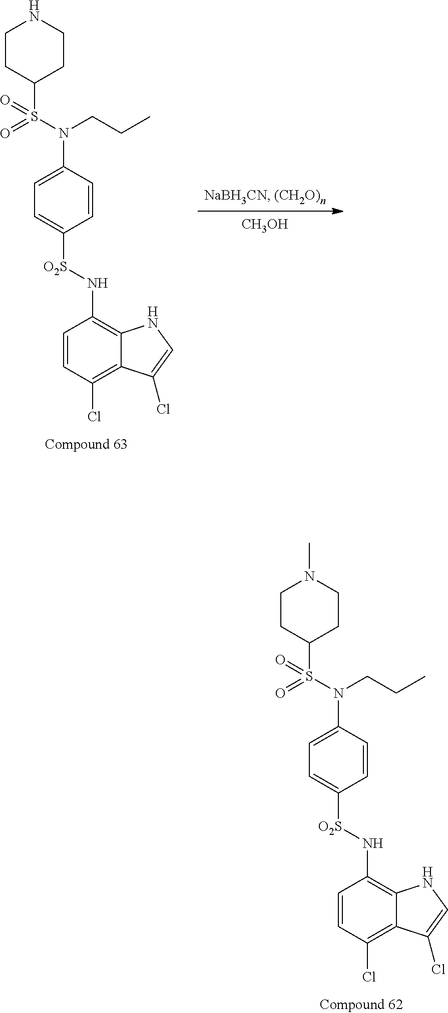 [SMILES: CCCN(c1ccc(S(=O)(=O)Nc2ccc(Cl)c3c(Cl)c[nH]c23)cc1)S(=O)(=O)C1CCNCC1 ; "CH3OH" ; "NaBH3CN, (CH2O)n" ; CCCN(c1ccc(S(=O)(=O)Nc2ccc(Cl)c3c(Cl)c[nH]c23)cc1)S(=O)(=O)C1CCN(C)CC1]

### 1. Preparation of N-(4-(N-(3,4-dichloro-1H-indol-7-yl)sulfamoyl)phenyl)-N-propylpiperidine-4-sulfonamide

To a solution of N-(4-(N-(3,4-dichloro-1H-indol-7-yl)sulfamoyl)phenyl)-N-propylpiperidine-4-sulfonamide (20.0 mg, 0.037 mmol) in CH₃OH (2 mL) were added a drop of AcOH, paraformaldehyde (3.3 mg, 0.11 mmol) and NaBH₃CN (4.7 mg, 0.074 mmol). The mixture was stirred at rt for 3 h. The mixture was poured into sat. aqueous NH₄Cl (10 mL) and extracted with EA (20 mL×3). The combined organic layer was dried over Na₂SO₄ and concentrated to give a residue, which was purified by prep-HPLC to afford N-(4-(N-(3,4-dichloro-1H-indol-7-yl)sulfamoyl)phenyl)-1-methyl-N-propylpiperidine-4-sulfonamide (HCOOH salt) (Compound 62, 3.2 mg, yield: 15.47%) as a white solid. LCMS (ESI) found: 559 [M+H]⁺. ¹H NMR (400 MHz, DMSO-d6) δ 11.28 (s, 1H), 8.22 (s, 1.5H), 7.70 (d, J=8.6 Hz, 2H), 7.46 (d, J=8.6 Hz, 2H), 7.33 (s, 1H), 6.75-6.71 (m, 2H), 3.66 (t, J=7.0 Hz, 2H), 3.02-2.96 (m, 1H), 2.80-2.74 (m, 2H), 2.13 (s, 3H), 1.89-1.81 (m, 4H), 1.63-1.51 (m, 2H), 1.28-1.21 (m, 2H), 0.77 (t, J=7.4 Hz, 3H).

### Example 30: Preparation of N-(4-(N-(3,4-dichloro-1H-indol-7-yl)sulfamoyl)phenyl)-N-propylpiperidine-4-sulfonamide; Compound 63

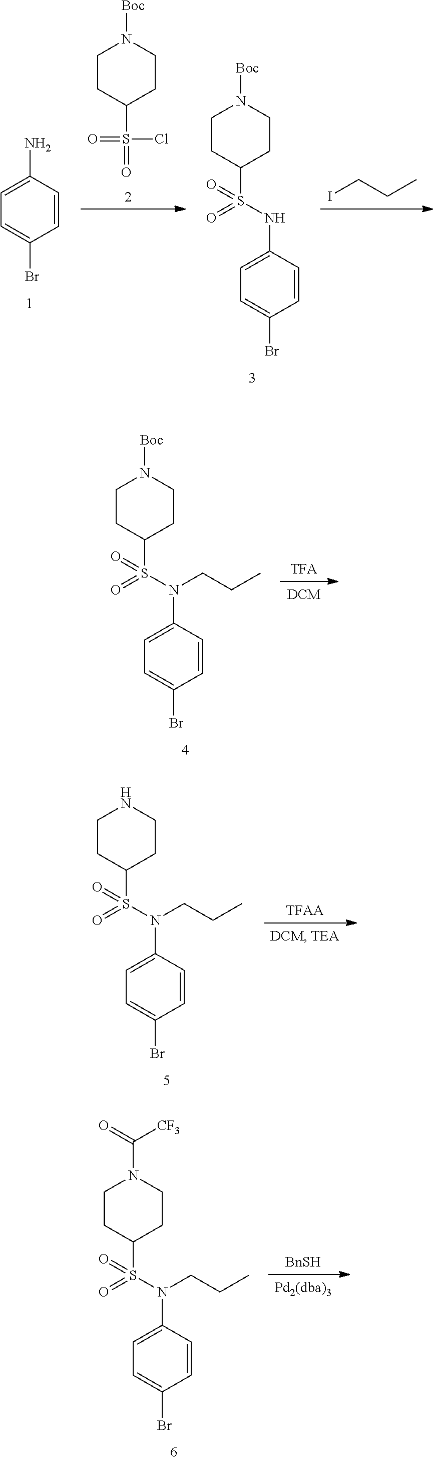

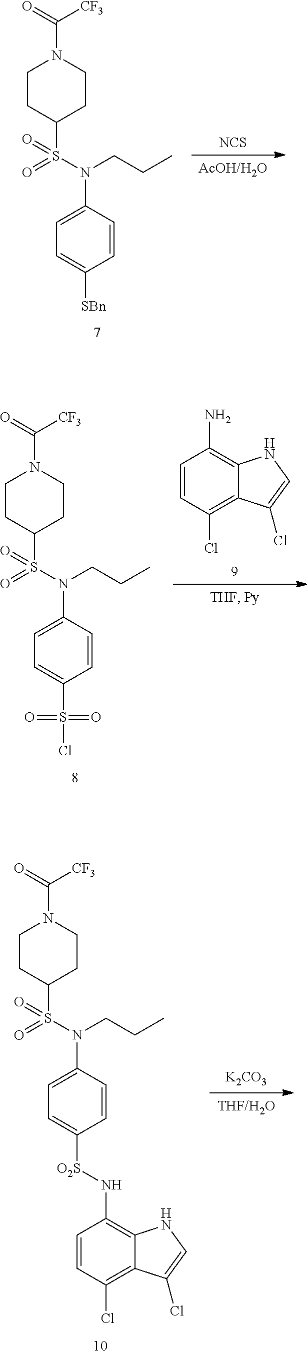

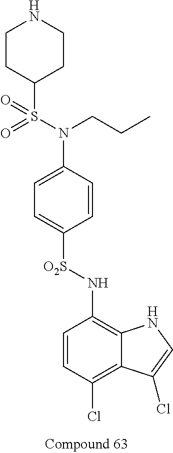 [SMILES: CCCN(c1ccc(S(=O)(=O)Nc2ccc(Cl)c3c(Cl)c[nH]c23)cc1)S(=O)(=O)C1CCNCC1]

### 1. Preparation of tert-butyl4-(N-(4-bromophenyl)sulfamoyl)piperidine-1-carboxylate

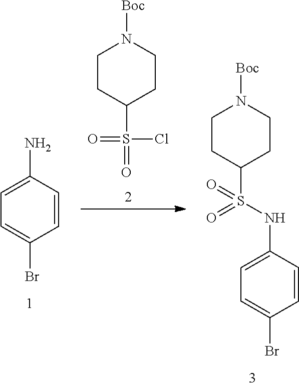

To a solution of 4-bromoaniline (650.0 mg, 3.80 mmol) and Py (900.0 mg, 11 mmol) in THF (5 mL) was added tert-butyl 4-(chlorosulfonyl)piperidine-1-carboxylate (1.08 g, 3.80 mmol). The mixture was stirred at rt for 2 h. The mixture was concentrated and purified by flash column to afford tert-butyl 4-(N-(4-bromophenyl)sulfamoyl)piperidine-1-carboxylate (1.2 g, 75.4%) as a white solid. LCMS (ESI) found: 419 [M+H]⁺.

### 2. Preparation of tert-butyl 4-(N-(4-bromophenyl)-N-propylsulfamoyl)piperidine-1-carboxylate

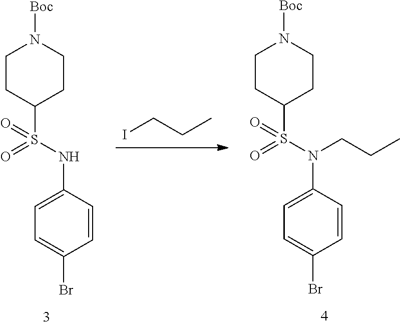

To a solution of tert-butyl 4-(N-(4-bromophenyl)sulfamoyl)piperidine-1-carboxylate (1.2 g, 2.82 mmol) in DMF (10 mL) was added sodium hydride (60%, 169.2 mg, 1.5 eq.) at 0° C. The mixture was stirred at rt for 30 min. Then iodopropane (959.0 mg, 5.64 mmol) was added. The mixture was stirred at room temperature for 3 h. The reaction was quenched with sat.aqueous NH₄Cl (20 mL) and extracted with EA (50 mL×3). The combined organic layer was concentrated to give a crude product, which was purified by flash to afford tert-butyl 4-(N-(4-bromophenyl)-N-propylsulfamoyl)piperidine-1-carboxylate (1.1 g, 84.6%) as a white solid. LCMS (ESI) found: 461 [M+H]⁺.

### 3. Preparation of N-(4-bromophenyl)-N-propylpiperidine-4-sulfonamide

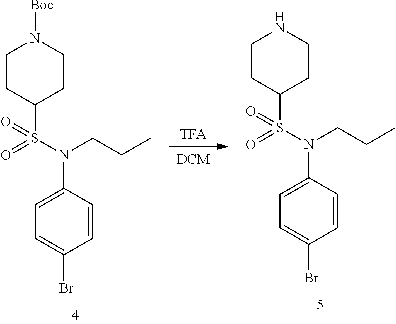

To a solution of 4-(N-(4-bromophenyl)-N-propylsulfamoyl)piperidine-1-carboxylate (1.1 g, 2.38 mmol) in DCM (10 mL) was slowly added TFA (2 mL). The mixture was stirred at rt for 3 h. Then the mixture was concentrated, basified with sat. aqueous NaHCO₃ (20 mL) to pH=8 and extracted with DCM (20 mL×3). The combined organic layer was concentrated and purified by flash column to afford N-(4-bromophenyl)-N-propylpiperidine-4-sulfonamide (760.0 mg, 88.5%) as a brown solid. LCMS (ESI) found: 361 [M+H]⁺.

### 4. Preparation of N-(4-bromophenyl)-N-propyl-1-(2,2,2-trifluoroacetyl)piperidine-4-sulfonamide

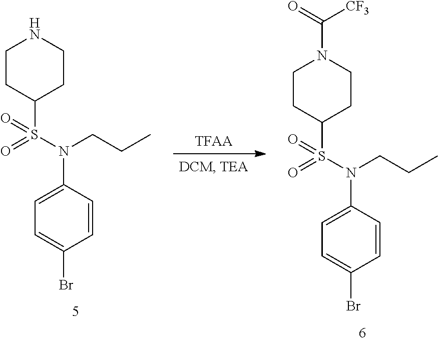 [SMILES: CCCN(c1ccc(Br)cc1)S(=O)(=O)C1CCNCC1 ; "TFAA" ; "DCM, TEA" ; CCCN(c1ccc(Br)cc1)S(=O)(=O)C1CCN(C(=O)C(F)(F)F)CC1]

To a solution of N-(4-bromophenyl)-N-propylpiperidine-4-sulfonamide (760.0 mg, 2.10 mmol in DCM (10 mL) were slowly added TEA (636.3 mg, 6.30 mmol) and TFAA (529.2 mg, 2.52 mmol) at 0° C. The mixture was stirred at rt for 1 h. The mixture was diluted with water (20 mL) and extracted with DCM (20 mL×3). The combined organic layer was concentrated and purified by flash column to afford N-(4-bromophenyl)-N-propyl-1-(2,2,2-trifluoroacetyl)piperidine-4-sulfonamide (860.0 mg, 89.6%) as a white solid. LCMS (ESI) found: 457 [M+H]⁺.

### 5. Preparation of N-(4-(benzylthio)phenyl)-N-propyl-1-(2,2,2-trifluoroacetyl)piperidine-4-sulfonamide

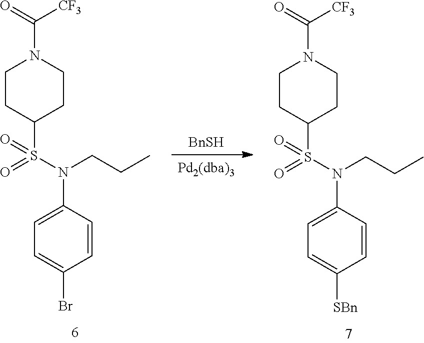

To a solution of N-(4-bromophenyl)-N-propyl-1-(2,2,2-trifluoroacetyl)piperidine-4-sulfonamide (390.0 mg, 0.86 mmol) in 1,4-dioxane (10 mL) were added BnSH (106.6 mg, 0.86 mmol), Pd₂(dba)₃ (79.0 mg, 0.086 mmol), Xantphos (98.3 mg, 0.17 mmol) and DIPEA (0.1 mL). The mixture was evaporated and backfilled with N₂ for three times. The mixture was stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give N-(4-(benzylthio)phenyl)-N-propyl-1-(2,2,2-trifluoroacetyl)piperidine-4-sulfonamide (365.0 mg, 84.7%) as a yellow solid. LCMS (ESI) found: 501 [M+H]⁺.

### 6. Preparation of 4-((N-propyl-1-(2,2,2-trifluoroacetyl)piperidine)-4-sulfonamido)benzenesulfonyl chloride

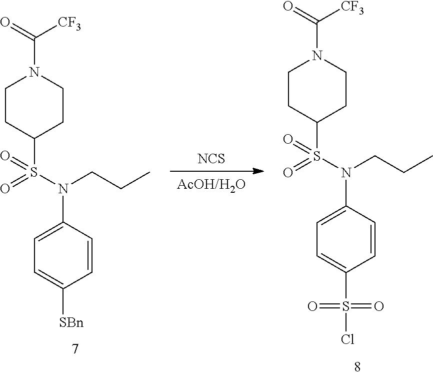

To a solution of N-(4-(benzylthio)phenyl)-N-propyl-1-(2,2,2-trifluoroacetyl)piperidine-4-sulfonamide (365.0 mg, 0.73 mmol) in AcOH/H₂O (9/1, 5 mL) was added NCS (487.0 mg, 3.65 mmol). The mixture was stirred at rt for 2 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 4-((N-propyl-1-(2,2,2-trifluoroacetyl)piperidine)-4-sulfonamido)benzenesulfonyl chloride (230.0 mg, 66.1%) as a white solid. LCMS (ESI) found: 477 [M+H]⁺.

### 7. Preparation of N-(4-(N-(3,4-dichloro-1H-indol-7-yl)sulfamoyl)phenyl)-N-propyl-1-(2,2,2-trifluoroacetyl)piperidine-4-sulfonamide

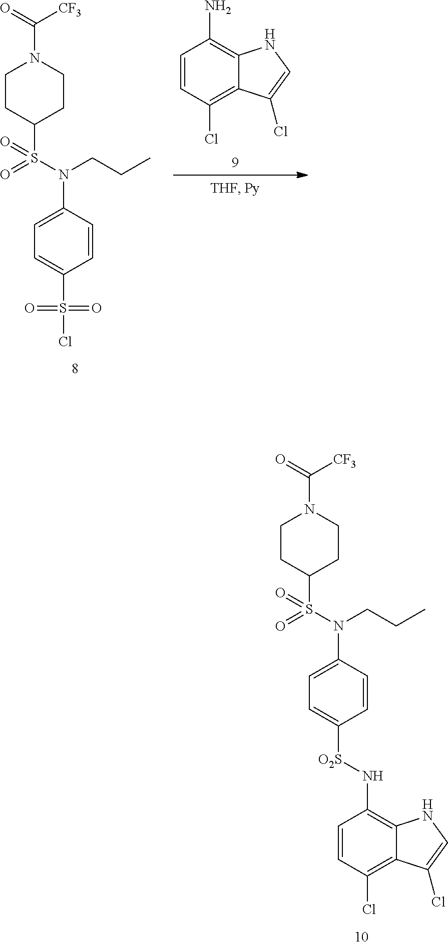

To a solution of 3,4-dichloro-1H-indol-7-amine (97 mg, 0.48 mmol) and pyridine (113.8 mg, 1.44 mmol) in dry THF (5 mL) was added 4-((N-propyl-1-(2,2,2-trifluoroacetyl)piperidine)-4-sulfonamido)benzenesulfonyl chloride (230.0 mg, 0.48 mmol). The mixture was stirred at r.t for 3 h. The mixture was concentrated and purified by prep-TLC to afford N-(4-(N-(3,4-dichloro-1H-indol-7-yl)sulfamoyl)phenyl)-N-propyl-1-(2,2,2-trifluoroacetyl)piperidine-4-sulfonamide (170.0 mg, 55.25%) as a white solid. LCMS (ESI) found: 641 [M+H]⁺.

### 8. Preparation of N-(4-(N-(3,4-dichloro-1H-indol-7-yl)sulfamoyl)phenyl)-N-propylpiperidine-4-sulfonamide

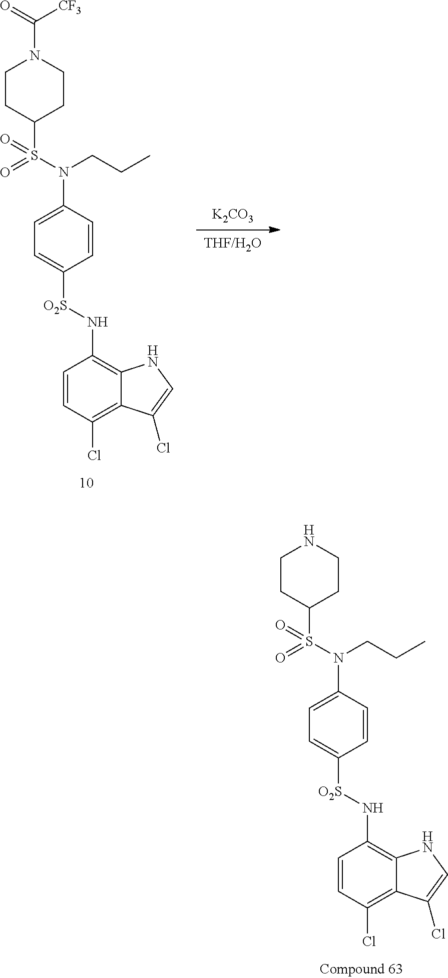 [SMILES: CCCN(c1ccc(S(=O)(=O)Nc2ccc(Cl)c3c(Cl)c[nH]c23)cc1)S(=O)(=O)C1CCN(C(=O)C(F)(F)F)CC1 ; "K2CO3" ; "THF/H2O" ; CCCN(c1ccc(S(=O)(=O)Nc2ccc(Cl)c3c(Cl)c[nH]c23)cc1)S(=O)(=O)C1CCNCC1]

To a solution of N-(4-(N-(3,4-dichloro-1H-indol-7-yl)sulfamoyl)phenyl)-N-propyl-1-(2,2,2-trifluoroacetyl)piperidine-4-sulfonamide (170.0 mg, 0.26 mmol) in THF/H₂O (4/1, 4 mL) was added K₂CO₃ (112.0 mg, 0.78 mmol). The reaction mixture was stirred at 50° C. for 3 h. The mixture was cooled and filtered. The filtrate was concentrated to give a residue, which was purified by prep-HPLC to afford N-(4-(N-(3,4-dichloro-1H-indol-7-yl)sulfamoyl)phenyl)-N-propylpiperidine-4-sulfonamide (HCOOH salt) (Compound 63, 85.0 mg, 59.9%) as a white solid. LCMS (ESI) found: 545 [M+H]⁺. ¹H NMR (400 MHz, DMSO-d6) δ 11.31 (s, 1H), 8.22 (s, 0.60H) 7.73 (d, J=8.6 Hz, 2H), 7.48 (d, J=8.6 Hz, 2H), 7.34 (s, 1H), 6.75-6.71 (m, 2H), 3.65 (t, J=7.0 Hz, 2H), 3.19-3.17 (m, 1H), 2.80-2.74 (m, 2H), 2.06-2.01 (m, 2H), 1.71-1.67 (m, 2H), 1.29-1.23 (m, 4H), 0.77 (t, J=7.4 Hz, 3H).

### Example 31: Preparation of N-(4-(N-(3,4-dichloro-1H-indol-7-yl)sulfamoyl)phenyl)-1-methylpiperidine-4-sulfonamide Compound 64

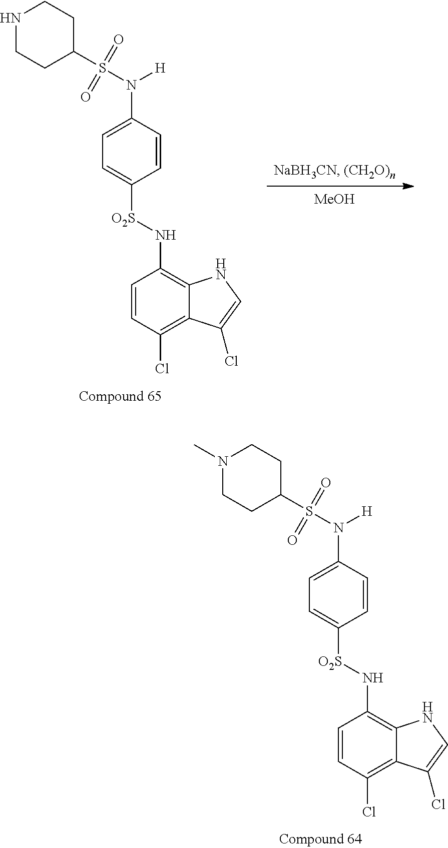 [SMILES: "MeOH" ; O=S(=O)(Nc1ccc(Cl)c2c(Cl)c[nH]c12)c1ccc(NS(=O)(=O)C2CCNCC2)cc1 ; "NaBH3CN, (CH2O)n" ; CN1CCC(S(=O)(=O)Nc2ccc(S(=O)(=O)Nc3ccc(Cl)c4c(Cl)c[nH]c34)cc2)CC1]

### 1. Preparation of N-(3,4-dichloro-1H-indol-7-yl)-4-((2-methyl-2-azaspiro[3.3]heptan-6-yl)oxy)benzenesulfonamide

To a solution of N-(4-(N-(3,4-dichloro-1H-indol-7-yl)sulfamoyl)phenyl)piperidine-4-sulfonamide (6 mg, 0.01 mmol) in MeOH (1 mL) were added a drop of AcOH and (CH₂O)ₙ (1.1 mg, 0.04 mmol). The mixture was stirred at rt for 30 min. NaBH₃CN (2.2 mg, 0.04 mmol) was added. The mixture was continued to stir at rt overnight. The mixture was concentrated to give a crude product, which was purified by prep-TLC eluted with DCM/MeOH=15/1 to give N-(4-(N-(3,4-dichloro-1H-indol-7-yl)sulfamoyl)phenyl)-1-methylpiperidine-4-sulfonamide (Compound 64, 4.5 mg, 73.0%) as a white solid. LCMS (ESI) found: 517 [M+H]⁺. ¹H NMR (400 MHz, DMSO-d6) δ 7.65 (d, J=8.8 Hz, 2H), 7.54 (s, 1H), 7.27 (d, J=8.8 Hz, 2H), 6.93 (d, J=8.2 Hz, 1H), 6.70 (d, J=8.2 Hz, 1H), 3.11-3.02 (m, 2H), 2.89-2.82 (m, 2H), 2.68 (s, 1H), 2.20 (s, 3H), 2.06-1.94 (m, 2H), 1.69-1.57 (m, 2H).

### Example 32: Preparation of N-(4-(N-(3,4-dichloro-1H-indol-7-yl)sulfamoyl)phenyl)piperidine-4-sulfonamide Compound 65

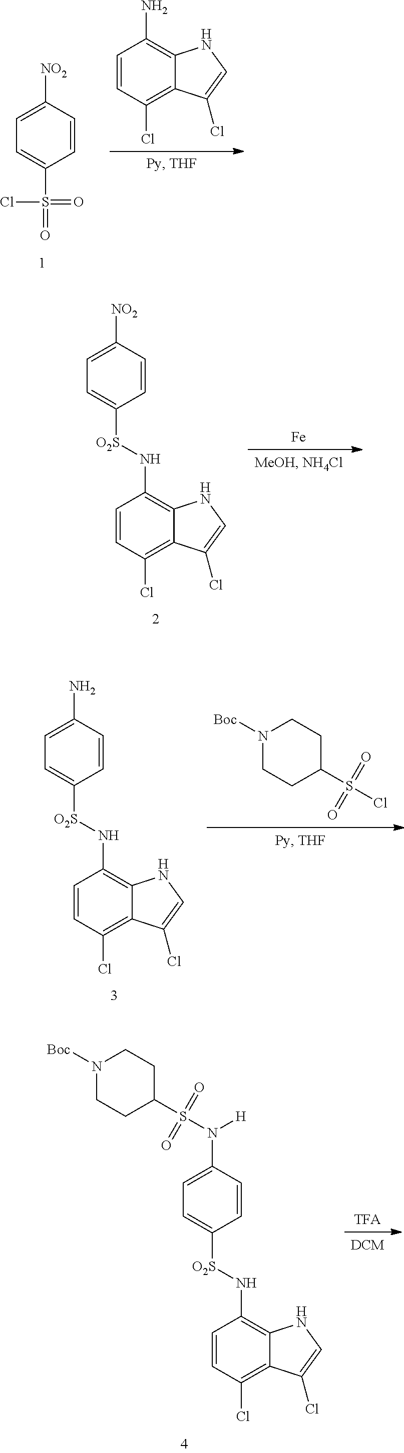

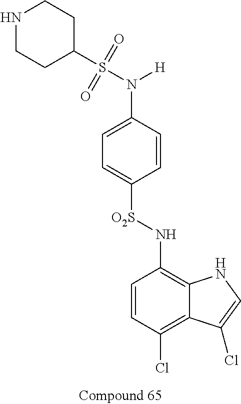 [SMILES: O=S(=O)(Nc1ccc(Cl)c2c(Cl)c[nH]c12)c1ccc(NS(=O)(=O)C2CCNCC2)cc1]

### 1. Preparation of N-(3,4-dichloro-1H-indol-7-yl)-4-nitrobenzenesulfonamide

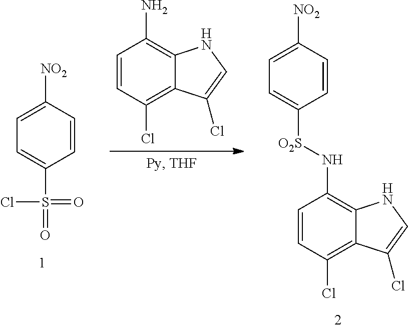

To a solution of 3,4-dichloro-1H-indol-7-amine (120 mg, 0.60 mmol) in THF (2 mL) were added pyridine (142.2 mg, 1.80 mmol) and 4-nitrobenzenesulfonyl chloride (133.0 mg, 0.60 mmol). The mixture was stirred at rt for 2 h. The mixture was concentrated and purified by flash to give N-(3,4-dichloro-1H-indol-7-yl)-4-nitrobenzenesulfonamide (200.0 mg, 86.7%) as a white solid. LCMS (ESI) found: 386 [M+H]⁺.

### 2. Preparation of 4-amino-N-(3,4-dichloro-1H-indol-7-yl)benzenesulfonamide

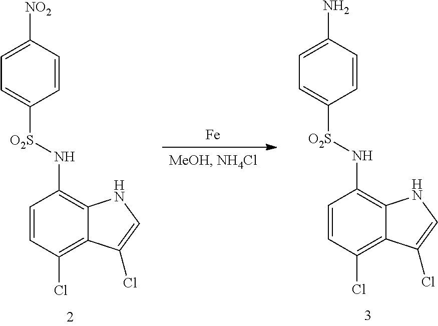

To a solution of N-(3,4-dichloro-1H-indol-7-yl)-4-nitrobenzenesulfonamide (200.0 mg, 0.52 mmol) in MeOH/NH₄Cl=1/1 (3 mL) was added Fe (145.6 mg, 2.60 mmol). The mixture was stirred for 1 h at 80° C. TLC showed SM has consumed. The reaction mixture was diluted with water (20 mL) and extracted with EA (30 mL×3). The combined organic layer was washed with water (20 mL), brine (20 mL), dried over Na₂SO₄ and concentrated under reduced pressure to afford a crude product, which was purified by column chromatography on silica gel eluted with DCM/MEOH (pure DCM to 10/1) to give 4-amino-N-(3,4-dichloro-1H-indol-7-yl)benzenesulfonamide (150.0 mg, 82.7%) as a white solid. LCMS (ESI) found: 356 [M+H]⁺.

### 3. Preparation of tert-butyl 4-(N-(4-(N-(3,4-dichloro-1H-indol-7-yl)sulfamoyl)phenyl)sulfamoyl)piperidine-1-carboxylate

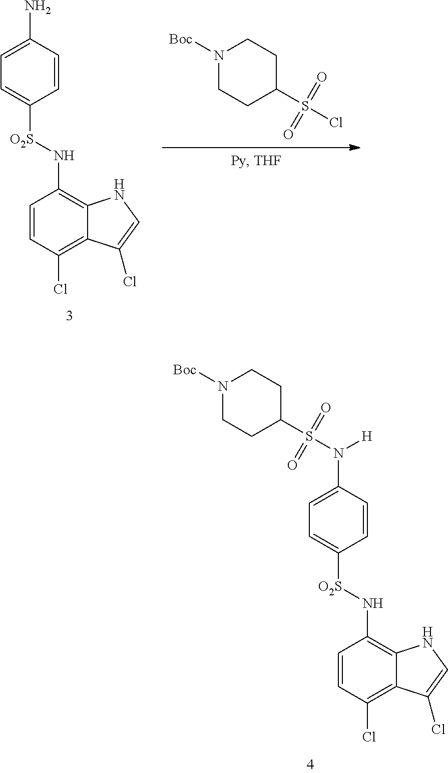

To a solution of 4-amino-N-(3,4-dichloro-1H-indol-7-yl)benzenesulfonamide (120.0 mg, 0.41 mmol) and pyridine (97.2 mg, 1.23 mmol) in THF (2 mL) was added tert-butyl 4-(chlorosulfonyl)piperidine-1-carboxylate (116.5 mg, 0.41 mmol). The mixture was stirred at rt for 1 h. The reaction mixture was diluted with water (20 mL) and extracted with EA (25 mL×3). The combined organic layer was washed with water (20 mL), brine (20 mL), dried over Na₂SO₄ and then concentrated under reduced pressure to afford a crude product, which was purified by column chromatography on silica gel eluted with PE/EA (10/1 to 2/1) to give tert-butyl 4-(N-(4-(N-(3,4-dichloro-1H-indol-7-yl)sulfamoyl)phenyl)sulfamoyl)piperidine-1-carboxylate (70.0 mg, 31.6%) as a white solid. LCMS (ESI) found: 603 [M+H]⁺.

### 4. Preparation of tert-butyl 4-(N-(4-(N-(3,4-dichloro-1H-indol-7-yl)sulfamoyl)phenyl)sulfamoyl)piperidine-1-carboxylate

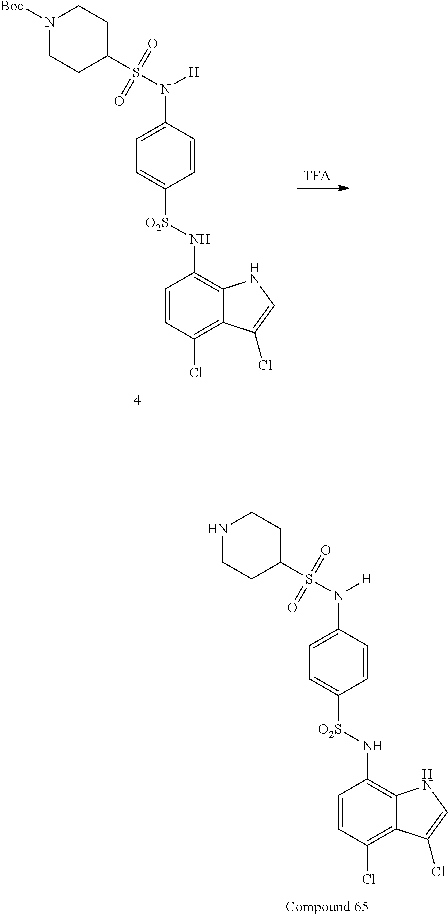 [SMILES: "TFA" ; CC(C)(C)OC(=O)N1CCC(S(=O)(=O)Nc2ccc(S(=O)(=O)Nc3ccc(Cl)c4c(Cl)c[nH]c34)cc2)CC1 ; O=S(=O)(Nc1ccc(Cl)c2c(Cl)c[nH]c12)c1ccc(NS(=O)(=O)C2CCNCC2)cc1]

To a solution of 4-(N-(4-(N-(3,4-dichloro-1H-indol-7-yl)sulfamoyl)phenyl)sulfamoyl)piperidine-1-carboxylate (20.0 mg, 0.03 mmol) in DCM (1 mL) was added TFA (0.5 mL). The mixture was stirred at rt for 2 h. The mixture was concentrated and basified with NaHCO₃ (20 mL) and extracted with DCM (20 mL×3). The combined organic layer was concentrated and purified by reversed phase flash eluting with CH₃OH/H₂O from 2/98 to 95/5 to give N-(4-(N-(3,4-dichloro-1H-indol-7-yl)sulfamoyl)phenyl)piperidine-4-sulfonamide (10.0 mg, 59.9%) as a white solid. LCMS (ESI) found: 503 [M+H]⁺. 4.0 mg was submitted as Compound 65, and the rest 6.0 mg was used for the SM for the preparation of compound 64. ¹H NMR (400 MHz, DMSO-d6) δ 7.43 (d, J=7.0 Hz, 2H), 7.14 (s, 1H), 6.89 (s, 2H), 6.64-6.48 (m, 2H), 2.94-2.88 (m, 2H), 2.84-2.79 (m, 1H), 2.35-2.22 (m, 2H), 1.83-1.76 (m, 2H), 1.62 (s, 1H), 1.48-1.33 (m, 2H).

### Example 33: Preparation of N-(4-(N-(3,4-dichloro-1H-indol-7-yl)sulfamoyl)phenyl)-4-methyl-N-propylpiperazine-1-sulfonamide; Compound 66

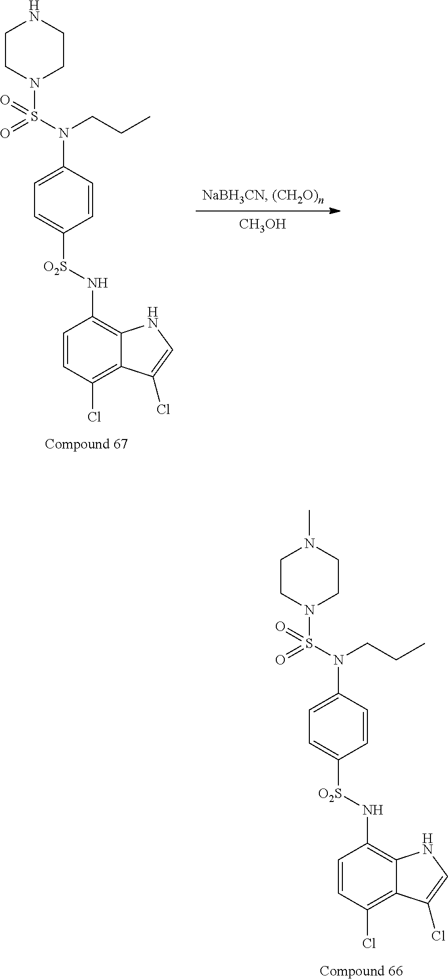 [SMILES: CCCN(c1ccc(S(=O)(=O)Nc2ccc(Cl)c3c(Cl)c[nH]c23)cc1)S(=O)(=O)N1CCNCC1 ; "CH3OH" ; "NaBH3CN, (CH2O)n" ; CCCN(c1ccc(S(=O)(=O)Nc2ccc(Cl)c3c(Cl)c[nH]c23)cc1)S(=O)(=O)N1CCN(C)CC1]

### 1. Preparation of N-(4-(N-(3,4-dichloro-1H-indol-7-yl)sulfamoyl)phenyl)-4-methyl-N-propylpiperazine-1-sulfonamide

To a solution of N-(4-(N-(3,4-dichloro-1H-indol-7-yl)sulfamoyl)phenyl)-N-propyl piperazine-1-sulfonamide (40.0 mg, 0.073 mmol) in THF (15 mL) were added (CH₂O)ₙ (4.4 mg, 0.14 mmol) and NaBH₃CN (9.2 mg, 0.15 mmol). The mixture was stirred for 2 h. The reaction mixture was concentrated and purified by prep-HPLC to give N-(4-(N-(3,4-dichloro-1H-indol-7-yl)sulfamoyl)phenyl)-4-methyl-N-propylpiperazine-1-sulfonamide (HCOOH salt) (Compound 66, 31.5 mg, 76.0%) as a white solid. LCMS (ESI): 560 [M+H]⁺. ¹H NMR (400 MHz, DMSO-d6) δ 11.36 (s, 1H), 8.14 (s, 0.30H), 7.71 (d, J=8.7 Hz, 2H), 7.56 (d, J=8.7 Hz, 2H), 7.53 (d, J=2.5 Hz, 1H), 6.91 (d, J=8.2 Hz, 1H), 6.66 (d, J=8.2 Hz, 1H), 3.65 (s, 2H), 3.02-2.95 (m, 4H), 2.26-2.17 (m, 4H), 2.13 (s, 3H), 1.31-1.26 (m, 2H), 0.78 (t, J=7.3 Hz, 3H).

### Example 34: Preparation of N-(4-(N-(3,4-dichloro-1H-indol-7-yl)sulfamoyl)phenyl)-N-propylpiperazine-1-sulfonamide; Compound 67

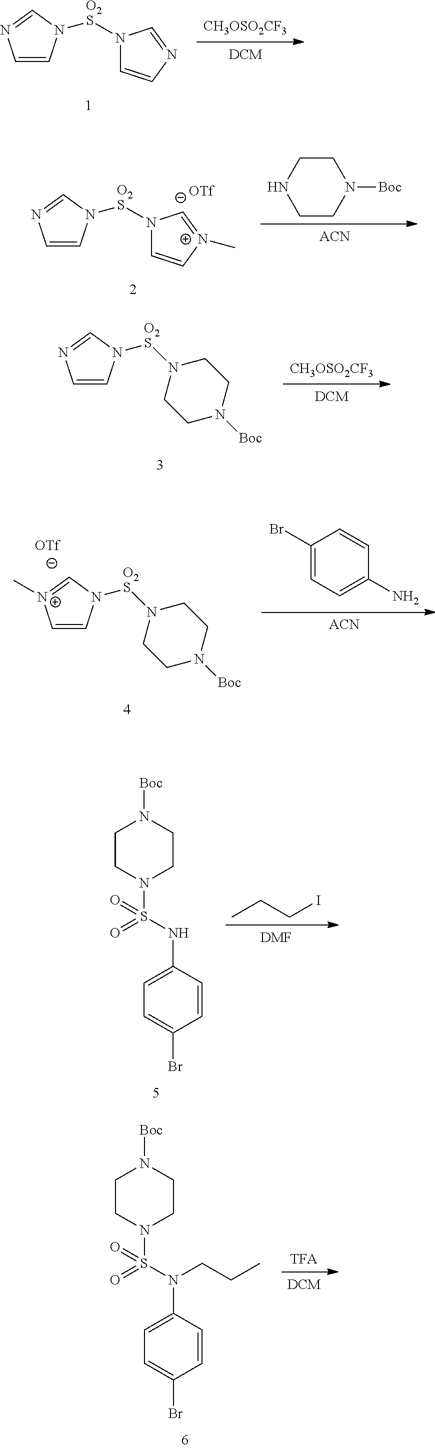

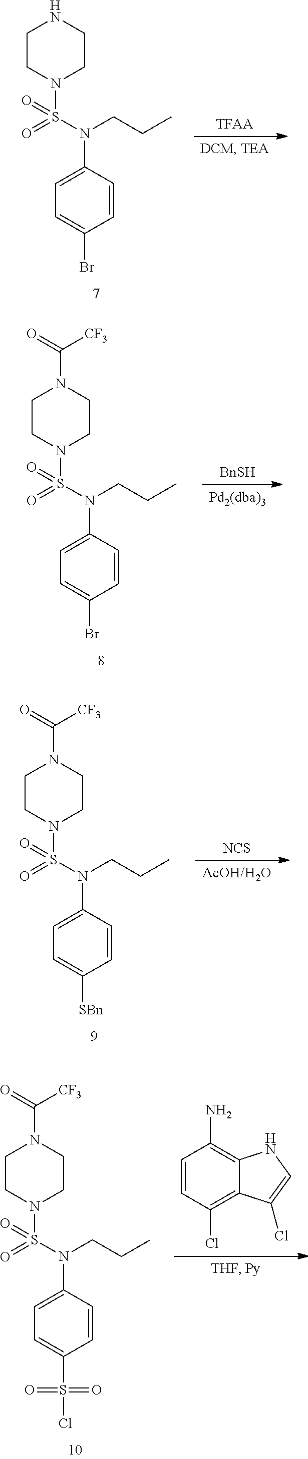

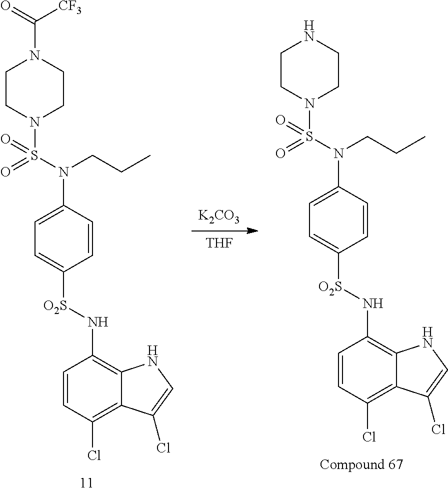 [SMILES: CCCN(c1ccc(S(=O)(=O)Nc2ccc(Cl)c3c(Cl)c[nH]c23)cc1)S(=O)(=O)N1CCN(C(=O)C(F)(F)F)CC1 ; "K2CO3" ; CCCN(c1ccc(S(=O)(=O)Nc2ccc(Cl)c3c(Cl)c[nH]c23)cc1)S(=O)(=O)N1CCNCC1 ; "THF"]

### 1. Preparation of 1-((1H-imidazol-1-yl)sulfonyl)-3-methyl-1H-imidazol-3-ium trifluoro methanesulfonate

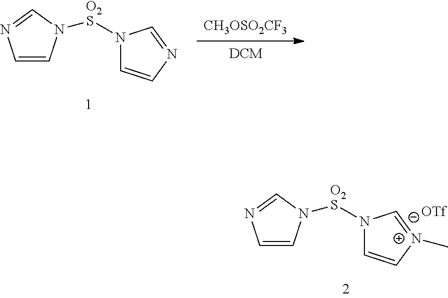

To a solution of 1,1′-sulfonylbis(1H-imidazole) (5.0 g, 25.25 mmol) in DCM (250 mL) was added CH₃OSO₂CF₃ (4.1 g, 25.25 mmol). The mixture was stirred at rt for 2 h. The mixture was filtered. The filtered cake was lyophilized to give crude 1-((1H-imidazol-1-yl)sulfonyl)-3-methyl-1H-imidazol-3-ium trifluoromethanesulfonate (5.5 g, 60.2%) as a white solid.

### 2. Preparation of tert-butyl 4-((1H-imidazol-1-yl)sulfonyl)piperazine-1-carboxylate

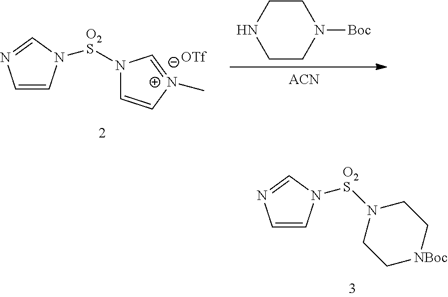

To a solution of 1-((1H-imidazol-1-yl)sulfonyl)-3-methyl-1H-imidazol-3-ium trifluoromethanesulfonate (5.5 g, 15.19 mmol) in DCM (20 mL), was added tert-butyl piperazine-1-carboxylate (2.8 g, 15.19 mmol). The mixture was stirred at 70° C. for 2 h. The mixture was concentrated to give a residue, which was purified by flash eluting with PE/EA from 30/1 to 25/1 to give tert-butyl 4-((1H-imidazol-1-yl)sulfonyl)piperazine-1-carboxylate (4.7 g, 99.6%) as a yellow solid. LCMS (ESI) found: 317 [M+H]⁻.

### 3. Preparation of ((trifluoromethyl)sulfonyl)-λ1-oxidane, 1-((4-(tert-butoxycarbonyl) piperazin-1-yl)sulfonyl)-3-methyl-1H-imidazol-3-ium salt

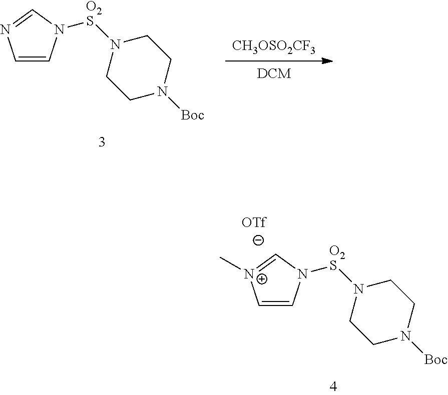

To a solution of tert-butyl 4-((1H-imidazol-1-yl)sulfonyl)piperazine-1-carboxylate (4.7 g, 15.10 mmol) in DCM (20 mL) was added CH₃OSO₂CF₃ (2.5 g, 15.10 mmol). The mixture was stirred at rt for 2 h. The mixture was filtered. The filtered cake was lyophilized to give the crude ((trifluoromethyl)sulfonyl)-λ1-oxidane, 1-((4-(tert-butoxycarbonyl) piperazin-1-yl)sulfonyl)-3-methyl-1H-imidazol-3-ium salt (4.5 g, 62.1%) as a white solid.

### 4. Preparation of tert-butyl 4-(N-(4-bromophenyl)sulfamoyl)piperazine-1-carboxylate

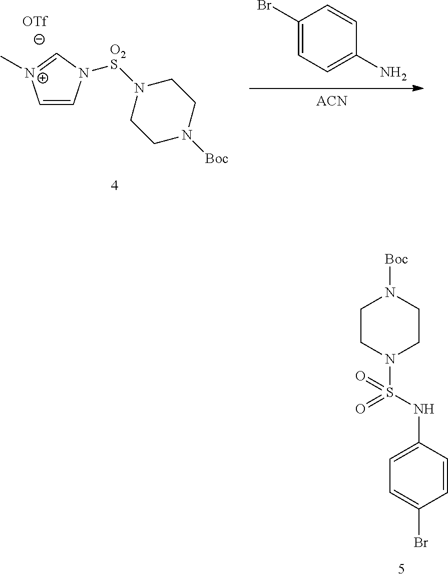

To a solution of ((trifluoromethyl)sulfonyl)-Xl-oxidane, 1-((4-(tert-butoxycarbonyl) piperazin-1-yl)sulfonyl)-3-methyl-1H-imidazol-3-ium salt (4.5 g, 9.37 mmol) in CH₃CN (20 mL) was added 4-bromoaniline (1.6 g, 9.37 mmol). The mixture was stirred at 70° C. for 2 h. The mixture was concentrated to give a residue, which was purified by flash eluting with PE/EA from 30/1 to 25/1 to give tert-butyl 4-(N-(4-bromophenyl)sulfamoyl) piperazine-1-carboxylate (3.8 g, 96.8%) as a yellow solid. LCMS (ESI) found: 420 [M+H]⁺.

### 5. Preparation of tert-butyl 4-(N-(4-bromophenyl)-N-propylsulfamoyl)piperazine-1-carboxylate

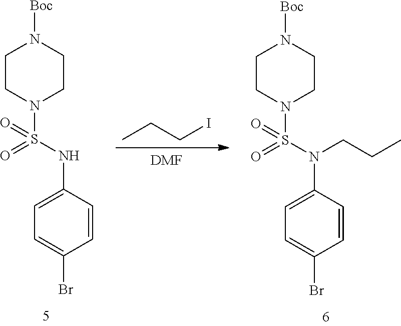

To a solution of tert-butyl 4-(N-(4-bromophenyl)sulfamoyl)piperazine-1-carboxylate (3.0 g, 7.14 mmol) in DMF (10 mL) was added NaH (60% in mineral oil, 285.6 mg, 7.14 mmol). The mixture was stirred at rt for 30 min. 1-iodopropane (1.2 g, 7.14 mmol) was added. The mixture was stirred at rt for another 2 h. Then the reaction mixture was quenched with sat. aqueous NH₄Cl (20 mL) and extracted with EA (25 mL×3). The combined organic layer was dried over MgSO₄, filtered and concentrated to give a residue, which was purified by flash to give tert-butyl 4-(N-(4-bromophenyl)-N-propylsulfamoyl)piperazine-1-carboxy late (3.1 g, 93.9%) as a white solid. LCMS (ESI) found: 462 [M+H]⁺.

### 6. Preparation of N-(4-bromophenyl)-N-propylpiperazine-1-sulfonamide

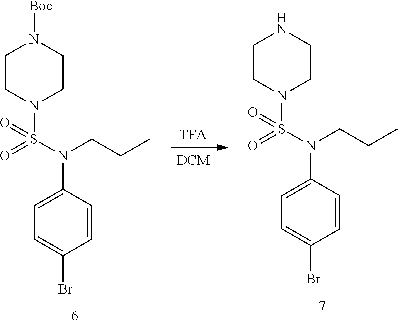

To a solution of tert-butyl 4-(N-(4-bromophenyl)-N-propylsulfamoyl)piperazine-1-carboxy late (3.1 g, 6.71 mmol) in DCM (10 mL) was added TFA (2 mL). The mixture was stirred at rt for 2 h. The mixture was concentrated to give crude N-(4-bromophenyl)-N-propyl piperazine-1-sulfonamide (TFA) (3.3 g, crude) as a brown oil. LCMS (ESI) found: 362 [M+H]⁺.

### 7. Preparation of N-(4-bromophenyl)-N-propyl-4-(2,2,2-trifluoroacetyl)piperazine-1-sulfon amide

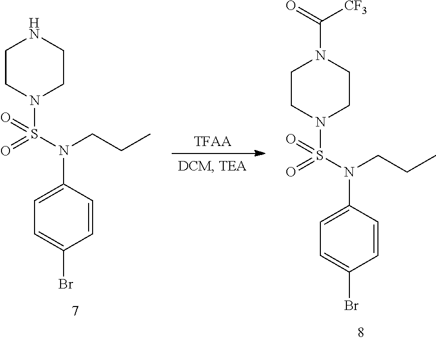 [SMILES: CCCN(c1ccc(Br)cc1)S(=O)(=O)N1CCNCC1 ; "TFAA" ; "DCM, TEA" ; CCCN(c1ccc(Br)cc1)S(=O)(=O)N1CCN(C(=O)C(F)(F)F)CC1]

To a solution of N-(4-bromophenyl)-N-propyl piperazine-1-sulfonamide (3.3 g, 6.71 mmol) in DCM (20 mL) were added TEA (2.0 g, 20.13 mmol) and TFAA (1.7 g, 8.05 mmol). The mixture was stirred at rt for 2 h. The reaction mixture was quenched with aqueous NaHCO₃ (20 mL), diluted with water (20 mL) and extracted with DCM (20 mL×3). The combined organic layer was dried over MgSO₄, filtered and concentrated to give a residue, which was purified by flash eluting with PE/EA from 25/1 to 20/1 to give N-(4-bromophenyl)-N-propyl-4-(2,2,2-trifluoro acetyl)piperazine-1-sulfonamide (3.1 g, 99.3%) as a yellow solid. LCMS (ESI) found: 458 [M+H]⁺.

### 8. Preparation of N-(4-(benzylthio)phenyl)-N-propyl-4-(2,2,2-trifluoroacetyl)piperazine-1-sulfonamide

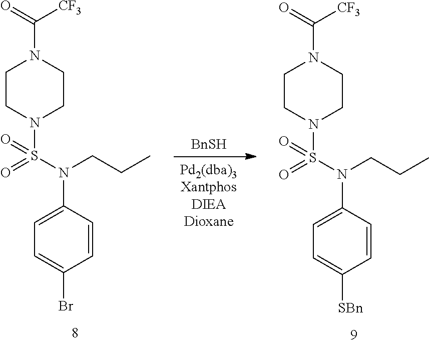

To a solution of N-(4-bromophenyl)-N-propyl-4-(trifluoroacetyl)piperazine-1-sulfon amide (3.1 g, 6.66 mmol) and BnSH (1.7 g, 13.32 mmol) in Dioxane (30 mL) were added Pd(dba)₃ (304.9 mg, 0.33 mmol), Xantphos (190.9 mg, 0.33 mmol) and DIPEA (859.1 mg, 6.66 mmol). The mixture was evaporated and backfilled with N₂ for three times. The mixture was stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give N-(4-(benzylthio)phenyl)-N-propyl-4-(2,2,2-trifluoroacetyl) piperazine-1-sulfonamide (2.8 g, 83.8%) as a green oil. LCMS (ESI) found: 502 [M+H]⁺.

### 9. Preparation of 4-((N-propyl-4-(2,2,2-trifluoroacetyl)piperazine)-1-sulfonamido)benzene sulfonyl chloride

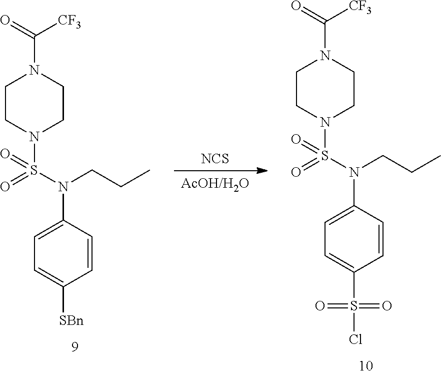

To a solution of N-(4-(benzylthio)phenyl)-N-propyl-4-(2,2,2-trifluoroacetyl)piperazine-1-sulfonamide (2.8 g, 5.58 mmol) in AcOH/H₂O (9:1, 25 mL) was added NCS (3.1 g, 23.44 mmol). The mixture was stirred at rt for 2 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 4-((N-propyl-4-(2,2,2-trifluoroacetyl)piperazine)-1-sulfonamido)benzenesulfonyl chloride (2.3 g, 86.2%) as a white solid. LCMS (ESI) found: 478 [M+H]⁺.

### 10. Preparation of N-(4-(N-(3,4-dichloro-1H-indol-7-yl)sulfamoyl)phenyl)-N-propyl-4-(2,2,2-trifluoroacetyl)piperazine-1-sulfonamide

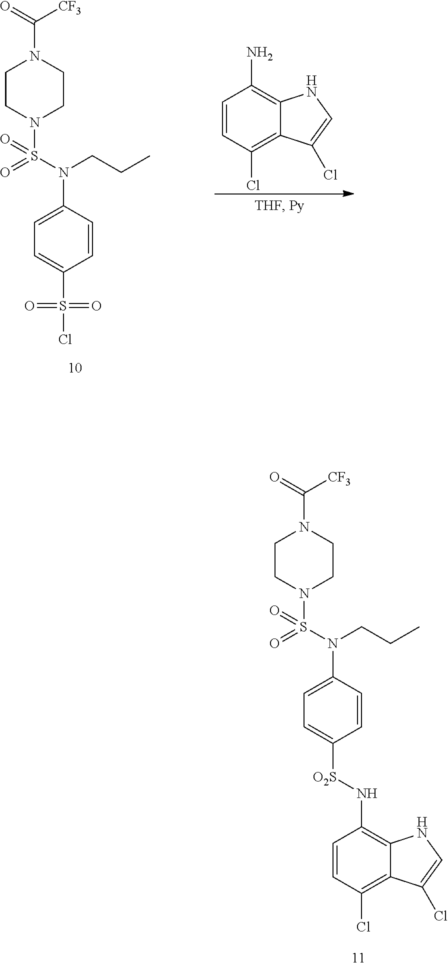 [SMILES: CCCN(c1ccc(S(=O)(=O)Cl)cc1)S(=O)(=O)N1CCN(C(=O)C(F)(F)F)CC1 ; Nc1ccc(Cl)c2c(Cl)c[nH]c12 ; CCCN(c1ccc(S(=O)(=O)Nc2ccc(Cl)c3c(Cl)c[nH]c23)cc1)S(=O)(=O)N1CCN(C(=O)C(F)(F)F)CC1]

To a solution of 3,4-dichloro-1H-indol-7-amine (92.5 mg, 0.46 mmol) and Pyridine (0.2 ml) in THF (5 mL) was added 4-((N-propyl-4-(2,2,2-trifluoroacetyl)piperazine)-1-sulfon amido)benzenesulfonyl chloride (250.0 mg, 0.52 mmol). The mixture was stirred at rt for 18 h. The mixture was concentrated to give a residue, which was purified by prep-HPLC to give N-(4-(N-(3,4-dichloro-1H-indol-7-yl)sulfamoyl)phenyl)-N-propyl-4-(2,2,2-trifluoroacetyl)piperazine-1-sulfonamide (200.0 mg, 67.4%) as a white solid. LCMS (ESI) found: 642 [M+H]⁺.

### 11. Preparation of N-(4-(N-(3,4-dichloro-1H-indol-7-yl)sulfamoyl)phenyl)-N-propylpiperazine-1-sulfonamide

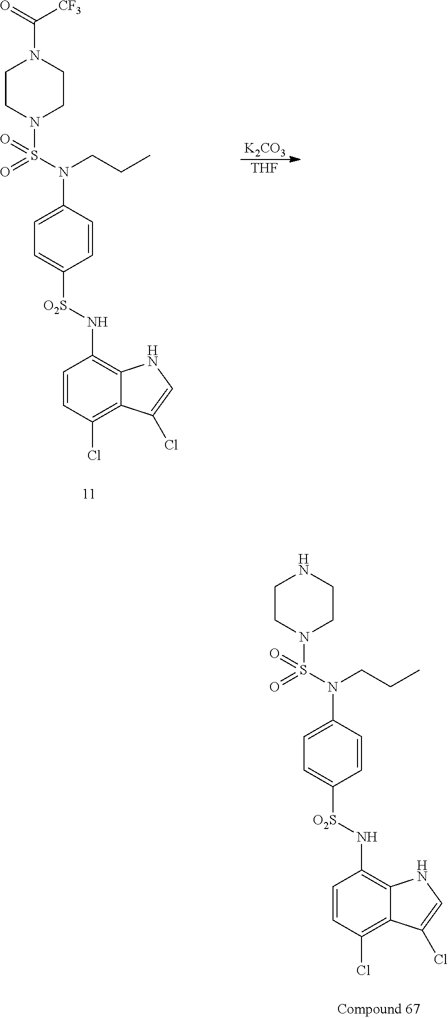

To a solution of N-(4-(N-(3,4-dichloro-1H-indol-7-yl)sulfamoyl)phenyl)-N-propyl-4-(2,2,2-trifluoroacetyl)piperazine-1-sulfonamide (200.0 mg, 0.31 mmol) in THF/H₂O (4/1, 10 mL) was added K₂CO₃ (214.2 mg, 1.55 mmol). The mixture was stirred at 70° C. for 2 h. The mixture was cooled and filtered. The filtrate was concentrated to give a residue, which was purified by prep-HPLC to give N-(4-(N-(3,4-dichloro-1H-indol-7-yl)sulfamoyl)phenyl)-N-propyl piperazine-1-sulfonamide (HCOOH salt) (Compound 67, 80.0 mg, 47.22%) as a white solid. LCMS (ESI) found: 546 [M+H]⁺. ¹HNMR (400 MHz, DMSO-d6) δ 11.45 (s, 1H), 7.75-7.68 (m, 2H), 7.52 (d, J=8.7 Hz, 2H), 7.48 (s, 1H), 6.89 (d, J=8.2 Hz, 1H), 6.66 (d, J=8.2 Hz, 1H), 3.62 (d, J=7.2 Hz, 2H), 3.07-2.98 (m, 4H), 2.78-2.66 (m, 4H), 1.32-1.27 (m, 2H), 0.78 (t, J=7.3 Hz, 3H).

### Example 35: Preparation of N-(3,4-dichloro-1H-indol-7-yl)-4-(N-ethylpiperidine-4-sulfonimidoyl)benzenesulfonamide; Compound 68

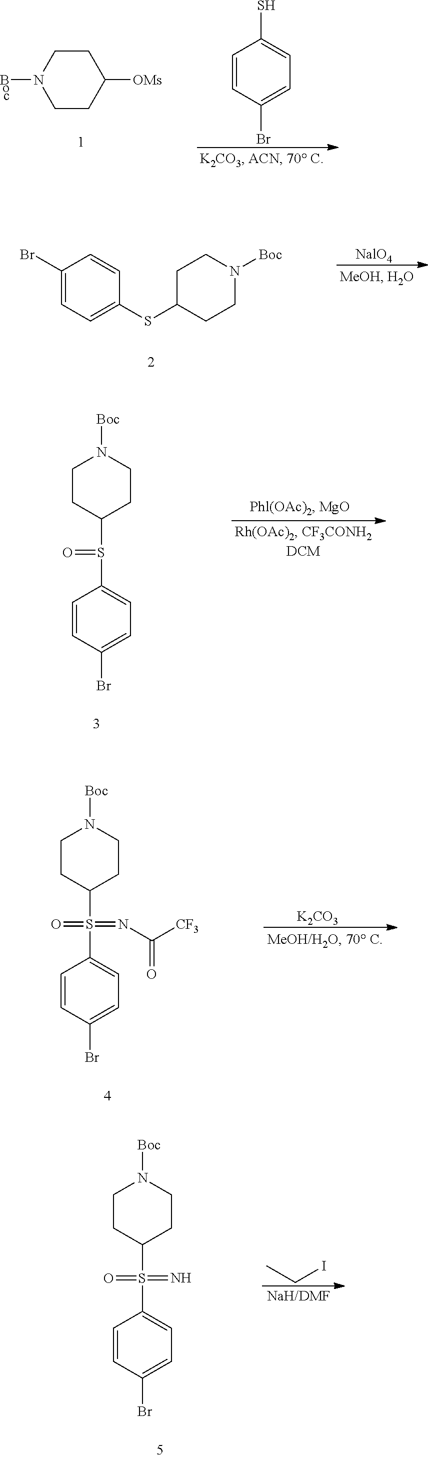

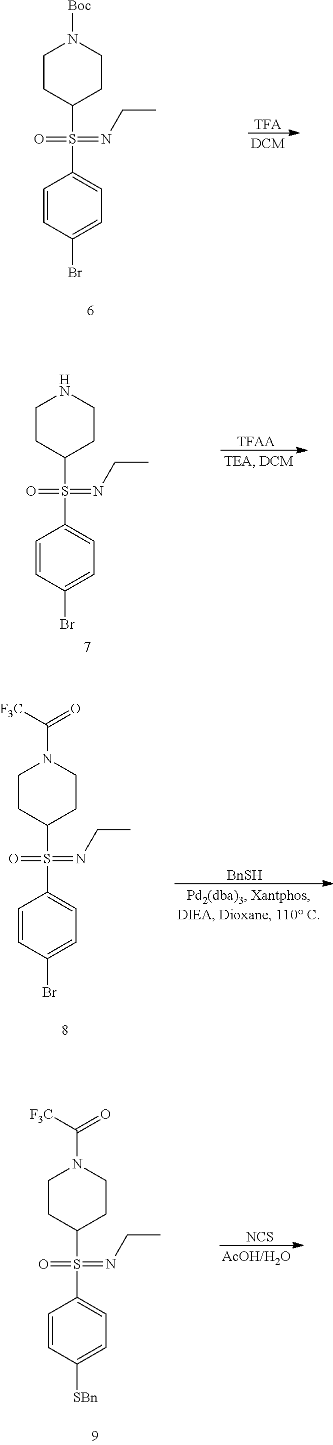

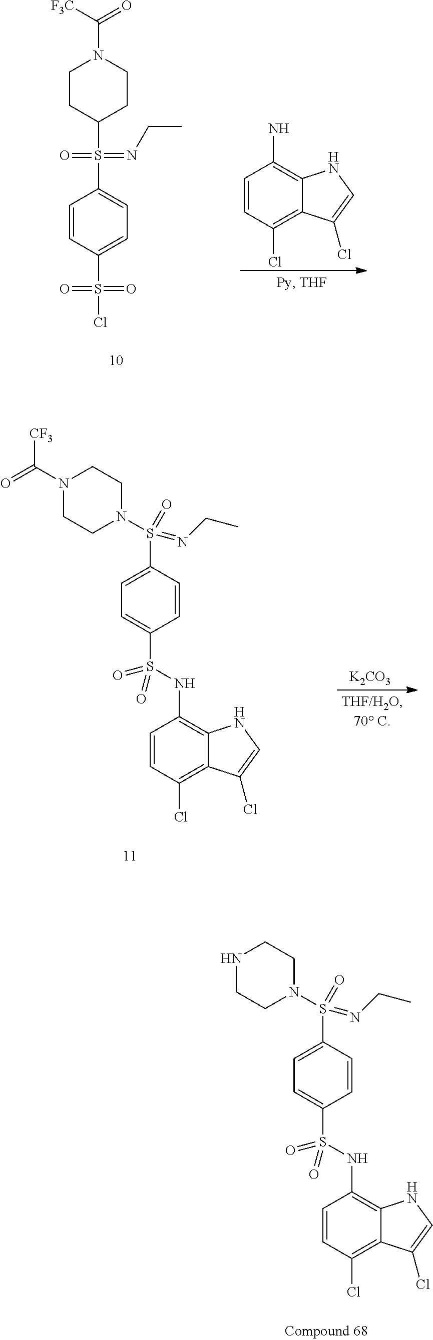 [SMILES: CCN=S(=O)(c1ccc(S(=O)(=O)Cl)cc1)C1CCN(C(=O)C(F)(F)F)CC1 ; Nc1ccc(Cl)c2c(Cl)c[nH]c12 ; CCN=S(=O)(c1ccc(S(=O)(=O)Nc2ccc(Cl)c3c(Cl)c[nH]c23)cc1)N1CCN(C(=O)C(F)(F)F)CC1 ; "K2CO3" ; CCN=S(=O)(c1ccc(S(=O)(=O)Nc2ccc(Cl)c3c(Cl)c[nH]c23)cc1)N1CCNCC1]

### 1. Preparation of tert-butyl 4-((4-bromophenyl)thio)piperidine-1-carboxylate

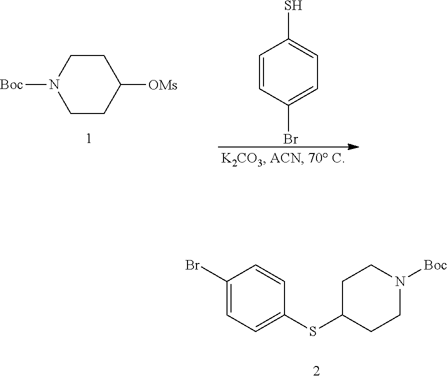 [SMILES: CC(C)(C)OC(=O)N1CCC(OS(C)(=O)=O)CC1 ; Sc1ccc(Br)cc1 ; CC(C)(C)OC(=O)N1CCC(Sc2ccc(Br)cc2)CC1]

To a solution of tert-butyl 4-(methanesulfonyloxy)piperidine-1-carboxylate (1.0 g, 3.58 mmol) in CH₃CN (30 mL) were added 4-bromobenzene-1-thiol (673.8 mg, 3.58 mmol) and K₂CO₃ (1.5 g, 10.74 mmol). The mixture was stirred at 70° C. for 18 h. The reaction mixture was quenched with aqueous NaHCO₃ (50 mL), diluted with water (100 mL) and extracted with EA (150 mL×3). The combined organic layer was dried over MgSO₄, filtered and concentrated to give a residue, which was purified by flash to give tert-butyl 4-((4-bromophenyl)thio) piperidine-1-carboxylate (800.0 mg, 60.1%) as a white solid. LCMS (ESI) found: 372 [M+H]⁺.

### 2. Preparation of tert-butyl 4-((4-bromophenyl)sulfinyl)piperidine-1-carboxylate

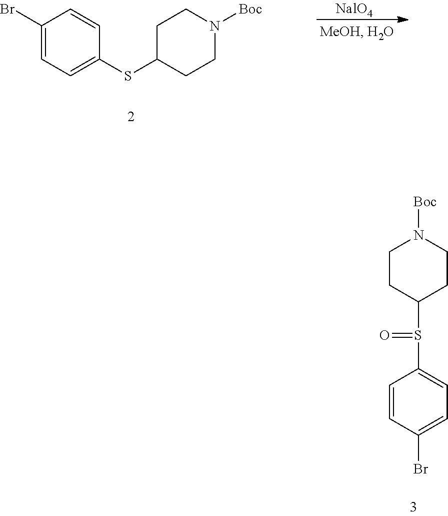 [SMILES: CC(C)(C)OC(=O)N1CCC(Sc2ccc(Br)cc2)CC1 ; CC(C)(C)OC(=O)N1CCC(S(=O)c2ccc(Br)cc2)CC1]

To a solution of tert-butyl 4-((4-bromophenyl)thio)piperidine-1-carboxylate (750.0 mg, 2.01 mmol) in CH₃OH/H₂O (2/1, 10 mL) was added NaIO₄ (862.9 mg, 4.02 mmol). The mixture was stirred at rt for 1 h. The reaction mixture was quenched with aqueous Na₂S₂O₃ (20 mL), diluted with water (20 mL) and extracted with EA (50 mL×3). The combined organic layer was dried over MgSO₄, filtered and concentrated to give a residue, which was purified by flash column to give tert-butyl 4-((4-bromophenyl)sulfinyl)piperidine-1-carboxylate (600.0 mg, 89.9%) as a white solid. LCMS (ESI) found: 388 [M+H]⁺.

### 3. Preparation of tert-butyl 4-(4-bromo-N-(2,2,2-trifluoroacetyl)phenylsulfonimidoyl)piperidine-1-carboxylate

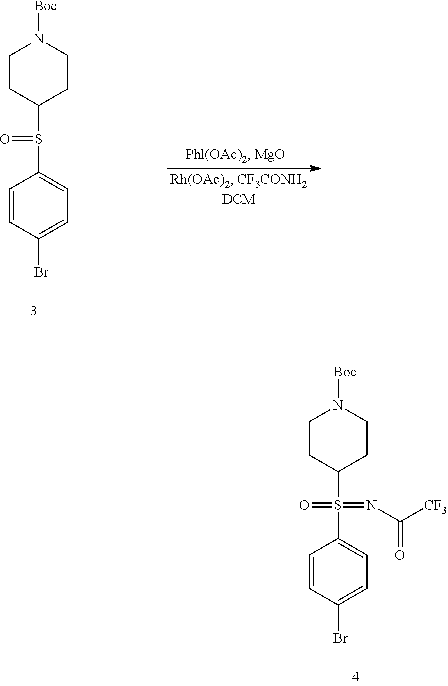 [SMILES: CC(C)(C)OC(=O)N1CCC(S(=O)c2ccc(Br)cc2)CC1 ; CC(C)(C)OC(=O)N1CCC(S(=O)(=NC(=O)C(F)(F)F)c2ccc(Br)cc2)CC1]

To a solution of tert-butyl 4-(4-bromobenzenesulfinyl)piperidine-1-carboxylate (600.0 mg, 1.54 mmol) and PhI(OAc)₂ (595.0 mg, 1.85 mmol) in DCM (10 mL) were added MgO-70 (184.5 mg, 4.62 mmol) and Rh(OAc)₂ (33.1 mg, 0.15 mmol), and CF₃CONH₂ (348.0 mg, 3.08 mmol). The mixture was stirred at rt for 5 h. LCMS showed the reaction was completed. The reaction mixture was concentrated in vacuum to remove most of solvent. The residue was purified by flash column eluting with PE/EA from 5/1 to 3/1 to give tert-butyl 4-(4-bromo-N-(2,2,2-trifluoroacetyl)phenylsulfonimidoyl)piperidine-1-carboxylate (530.0 mg, 68.8%) as yellow solid. LCMS (ESI) found: 499 [M+H]⁺.

### 4. Preparation of tert-butyl 4-(4-bromophenylsulfonimidoyl)piperidine-1-carboxylate

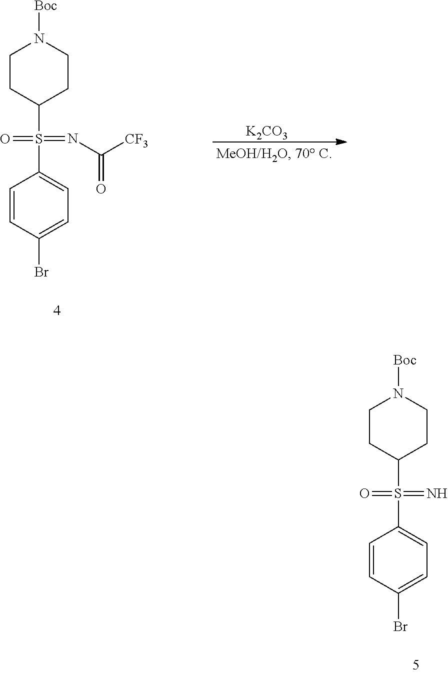 [SMILES: CC(C)(C)OC(=O)N1CCC(S(=O)(=NC(=O)C(F)(F)F)c2ccc(Br)cc2)CC1 ; "K2CO3" ; CC(C)(C)OC(=O)N1CCC(S(=N)(=O)c2ccc(Br)cc2)CC1]

To a solution of tert-butyl 4-(4-bromo-N-(2,2,2-trifluoroacetyl)phenylsulfonimidoyl) piperidine-1-carboxylate (530.0 mg, 1.06 mmol) in MeOH/H₂O (4:1, 10 mL) was added K₂CO₃ (438.8 mg, 3.18 mmol). The mixture was stirred for 2 h. Then the mixture was filtered. The filtrate was concentrated to give a residue, which was purified by flash column eluting with PE/EA from 2/1 to 1/1 to give tert-butyl 4-(4-bromophenylsulfonimidoyl)piperidine-1-carboxylate (400.0 mg, 93.4%) as white solid. LCMS (ESI) found: 403 [M+H]⁺.

### 5. Preparation of tert-butyl 4-(4-bromo-N-ethylphenylsulfonimidoyl)piperidine-1-carboxy late

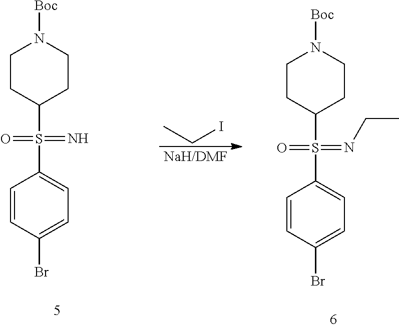

To a solution of tert-butyl 4-(4-bromophenylsulfonimidoyl)piperidine-1-carboxylate (360.0 mg, 0.89 mmol) in DMF (100 mL) was added NaH (71.3 mg, 1.78 mmol). The mixture was stirred at rt for 30 min. Iodoethane (277.7 mg, 1.78 mmol) was added at room temperature. The mixture was stirred at rt for 2 h. The reaction mixture was quenched with sat. aqueous NH₄Cl (2 mL), diluted with water (20 mL) and extracted with EA (25 mL×3). The organic layer was dried over MgSO₄, filtered and concentrated to give a residue, which was purified by flash column eluting with PE/EA from 15/1 to 10/1 to give tert-butyl 4-(4-bromo-N-ethylphenylsulfonimidoyl)piperidine-1-carboxylate (355.0 mg, 92.5%) as white solid. LCMS (ESI) found: 431 [M+H]⁺.

### 6. Preparation of (4-bromophenyl)(ethylimino)(piperidin-4-yl)-λ6-sulfanone

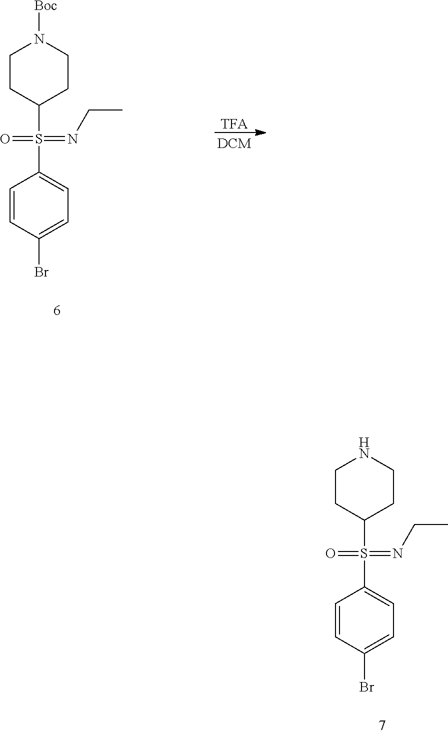 [SMILES: CCN=S(=O)(c1ccc(Br)cc1)C1CCN(C(=O)OC(C)(C)C)CC1 ; CCN=S(=O)(c1ccc(Br)cc1)C1CCNCC1 ; "TFA"]

To a solution of tert-butyl 4-(4-bromo-N-ethylphenylsulfonimidoyl)piperidine-1-carboxylate (355.0 mg, 0.82 mmol) in DCM (2 mL) was added TFA (1 mL). The mixture was stirred for 2 h. The mixture was concentrated and basified with Saturated aqueous NaHCO₃ (10 mL). The mixture was extracted with DCM (10 mL×3). The combined organic layer was concentrated to give (4-bromophenyl)(ethylimino)(piperidin-4-yl)-6-sulfanone (300.0 mg) as a brown oil. LCMS (ESI) found: 331 [M+H]⁺.

### 7. Preparation of (4-bromophenyl)(ethylimino)(1-(2,2,2-trifluoroacetyl)piperidin-4-yl)-6-sulfanone

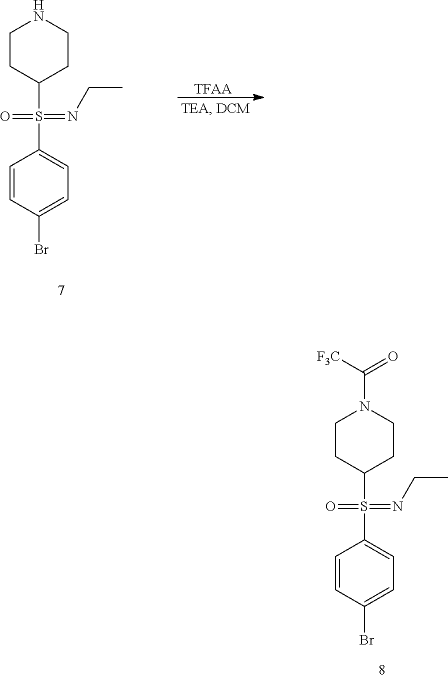 [SMILES: CCN=S(=O)(c1ccc(Br)cc1)C1CCNCC1 ; "TFAA" ; CCN=S(=O)(c1ccc(Br)cc1)C1CCN(C(=O)C(F)(F)F)CC1]

To a solution of (4-bromophenyl)(ethylimino)(piperidin-4-yl)-26-sulfanone (300.0 mg, 0.82 mmol) in DCM (5 mL) were added TEA (248.5 mg, 2.46 mmol) and TFAA (206.6 mg, 0.98 mol). The mixture was stirred at 0° C. for 1 h. The reaction mixture was poured into saturated aqueous NaHCO₃ (20 mL) slowly. The mixture extracted with DCM (20 mL×3). The combined organic layer was dried over MgSO₄, filtered and concentrated to give a residue, which was purified by flash column eluting with PE/EA from 30/1 to 25/1 to give (4-bromophenyl)(ethylimino)(1-(2,2,2-trifluoroacetyl) piperidin-4-yl)-6-sulfanone (180.0 mg, yield: 51.4%) as a yellow solid. LCMS (ESI) found: 427 [M+H]⁺.

### 8. Preparation of (4-(benzylthio)phenyl)(ethylimino)(1-(2,2,2-trifluoroacetyl) piperidin-4-yl)-6-sulfanone

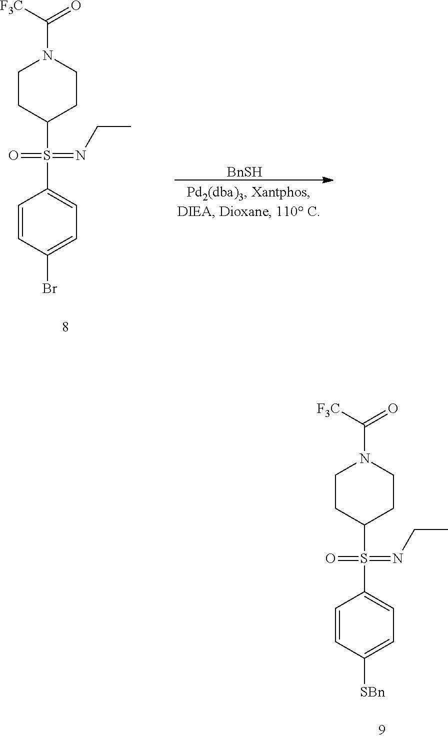

To a solution of (4-bromophenyl)(ethylimino)(1-(2,2,2-trifluoroacetyl)piperidin-4-yl)-6-sulfanone (180.0 mg, 0.42 mmol and BnSH (104.6 mg, 0.84 mmol) in Didoxane (4 mL) was added Pd₂(dba)₃ (38.5 mg, 0.042 mmol) and Xantphos (24.3 mg, 0.042 mmol). The mixture was evaporated and backfilled with N₂ for three times. The mixture was stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give (4-(benzylthio) phenyl)(ethylimino)(1-(2,2,2-trifluoroacetyl) piperidin-4-yl)-6-sulfanone (150.0 mg, 75.8%) as green oil. LCMS (ESI) found: 471 [M+H]⁺.

### 9. Preparation of 4-(N-ethyl-1-(2,2,2-trifluoroacetyl)piperidine-4-sulfonimidoyl)benzene sulfonyl chloride

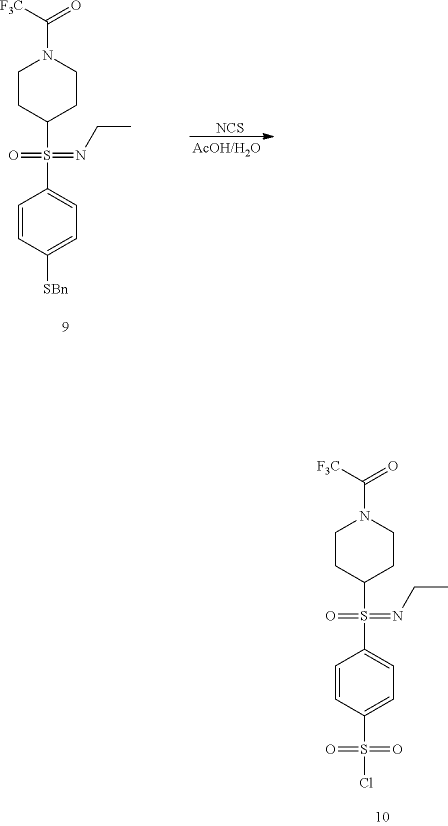

To a solution of (4-(benzylthio) phenyl)(ethylimino)(1-(2,2,2-trifluoroacetyl)piperidin-4-yl)-6-sulfanone (150.0 mg, 0.32 mmol) in AcOH/H₂O (9/1, 5 mL) was added NCS (180.1 mg, 1.34 mmol). The mixture was stirred at rt for 1 h. The mixture was stirred at rt for 2 h. The reaction was quenched with aqueous Na₂S₂₀₃ (15 mL) and extracted with DCM (15 mL×3). The combined organic layer was concentrated to give a crude product, which was purified by column chromatography on silica gel eluting with PE/EA from 15/1 to 5/1 to afford 4-(N-ethyl-1-(2,2,2-trifluoroacetyl)piperidine-4-sulfonimidoyl) benzenesulfonyl chloride (65.0 mg, 45.4%) as a white solid. LCMS (ESI) found: 447 [M+H]⁺.

### 10. Preparation of N-(3,4-dichloro-1H-indol-7-yl)-4-(N-ethyl-1-(2,2,2-trifluoroacetyl) piperidine-4-sulfonimidoyl)benzenesulfonamide

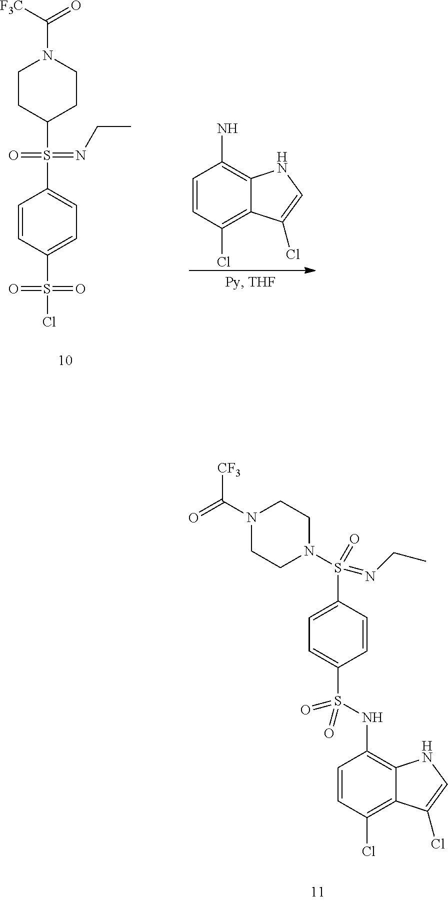 [SMILES: CCN=S(=O)(c1ccc(S(=O)(=O)Cl)cc1)C1CCN(C(=O)C(F)(F)F)CC1 ; Nc1ccc(Cl)c2c(Cl)c[nH]c12 ; CCN=S(=O)(c1ccc(S(=O)(=O)Nc2ccc(Cl)c3c(Cl)c[nH]c23)cc1)N1CCN(C(=O)C(F)(F)F)CC1]

To a solution of 3,4-dichloro-1H-indol-7-amine (20.0 mg, 0.10 mmol) and pyridine (30.8 mg, 0.39 mmol) in THF (2 mL) was added 4-(N-ethyl-1-(2,2,2-trifluoroacetyl)piperidine-4-sulfonimidoyl)benzenesulfonyl chloride (60.0 mg, 0.13 mmol). The mixture was stirred at rt for 18 h. The mixture was concentrated to give a crude product, which was purified by flash to give N-(3,4-dichloro-1H-indol-7-yl)-4-(N-ethyl-1-(2,2,2-trifluoroacetyl)piperidine-4-sulfonimidoyl) (30.0 mg, 49.0%) as a white solid. LCMS (ESI) found: 611 [M+H]⁺.

### 11. Preparation of N-(3,4-dichloro-1H-indol-7-yl)-4-(N-ethylpiperidine-4-sulfonimidoyl) benzenesulfonamide

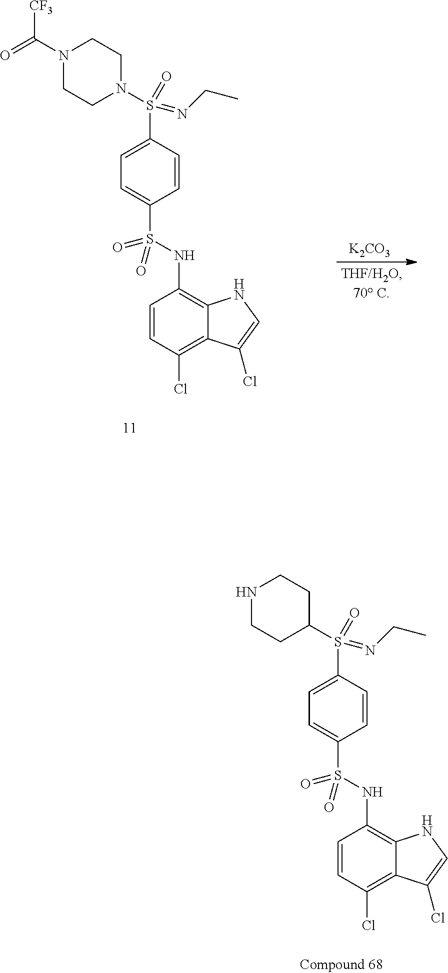 [SMILES: CCN=S(=O)(c1ccc(S(=O)(=O)Nc2ccc(Cl)c3c(Cl)c[nH]c23)cc1)N1CCN(C(=O)C(F)(F)F)CC1 ; "K2CO3" ; CCN=S(=O)(c1ccc(S(=O)(=O)Nc2ccc(Cl)c3c(Cl)c[nH]c23)cc1)C1CCNCC1]

To a solution of N-(3,4-dichloro-1H-indol-7-yl)-4-(N-ethyl-1-(2,2,2-trifluoroacetyl) piperidine-4-sulfonimidoyl) (30.0 mg, 0.05 mmol) in THF/H₂O (4/1, 5 mL) was added K₂CO₃ (34.6 mg, 0.25 mmol). The mixture was stirred at 70° C. for 2 h. The mixture was filtered and the filtrated was concentrated to give a crude product, which was purified by prep-HPLC to give N-(3,4-dichloro-1H-indol-7-yl)-4-(N-ethylpiperidine-4-sulfonimidoyl)benzene sulfonamide (HCOOH salt) (Compound 68, 13.1 mg, 50.0%) as a white solid. LCMS (ESI) found: 515 [M+H]⁺. ¹H NMR (400 MHz, DMSO-d6) δ 11.25 (s, 1H), 8.17 (s, 0.61H), 7.97 (d, J=8.4 Hz, 2H), 7.73 (d, J=8.4 Hz, 2H), 7.21 (s, 1H), 6.76 (d, J=8.2 Hz, 1H), 6.68 (d, J=8.2 Hz, 1H), 3.42-3.41 (m, 1H), 3.27-3.24 (m, 2H), 2.86-2.81 (m, 1H), 2.79-2.70 (m, 3H), 2.14-2.07 (m, 1H), 1.88-1.79 (m, 1H), 1.63-1.51 (m, 2H), 1.04 (t, J=7.2 Hz, 3H).

### Example 36: Preparation of N-(3,4-dichloro-1H-indol-7-yl)-4-(N-ethylpiperazine-1-sulfonimidoyl)benzenesulfonamide; Compound 69

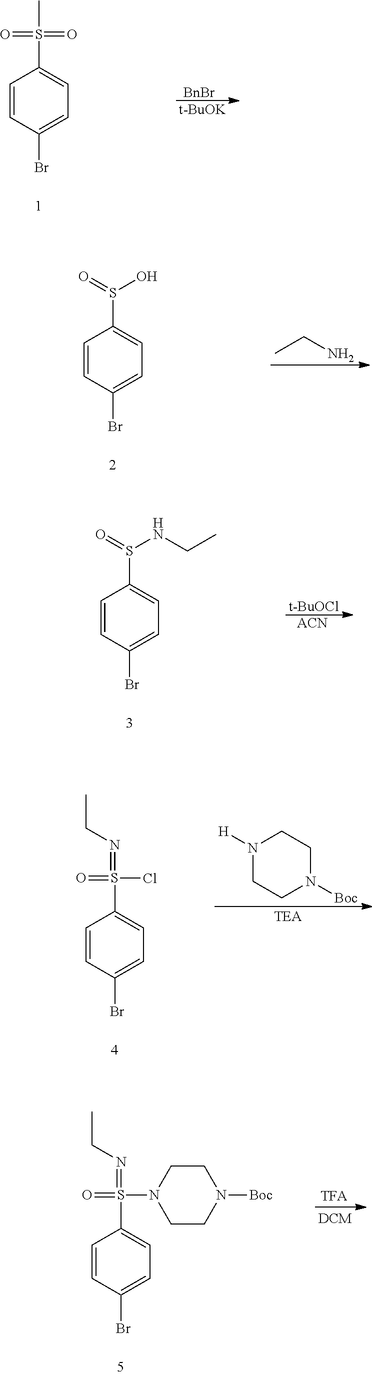

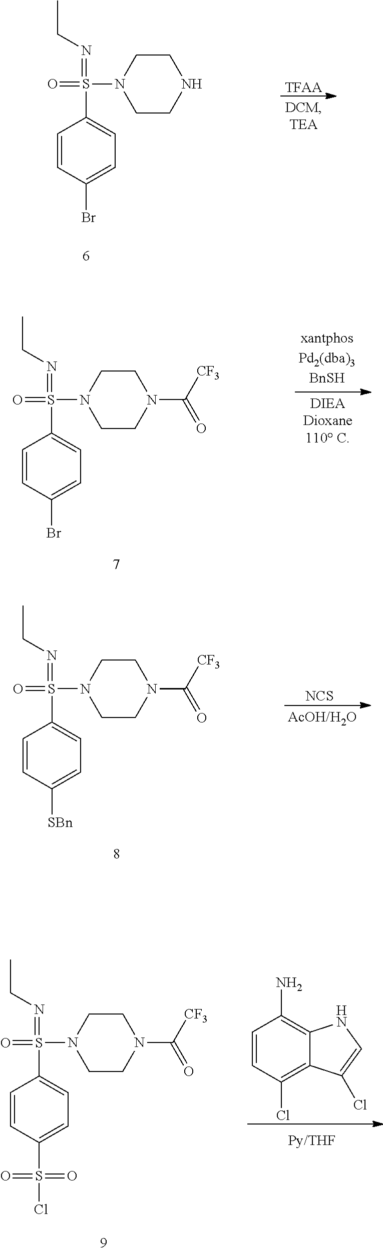

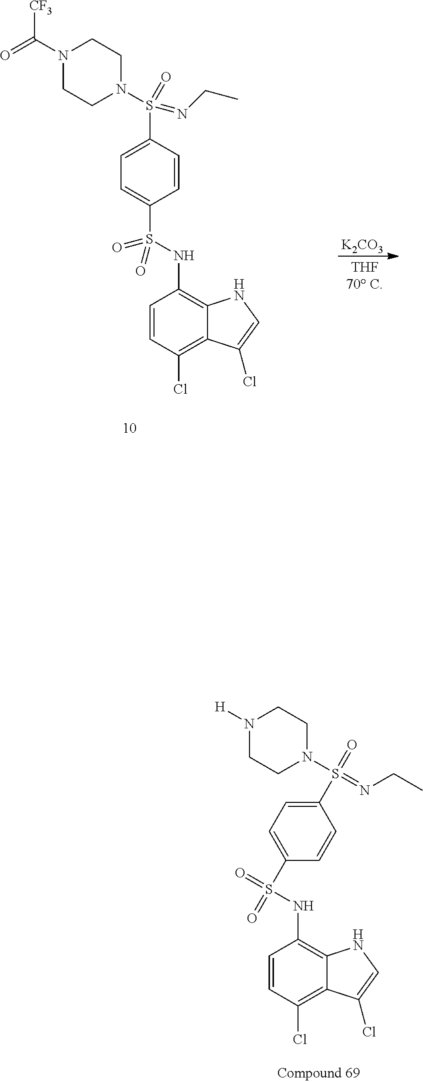 [SMILES: CCN=S(=O)(c1ccc(S(=O)(=O)Nc2ccc(Cl)c3c(Cl)c[nH]c23)cc1)N1CCN(C(=O)C(F)(F)F)CC1 ; "K2CO3" ; CCN=S(=O)(c1ccc(S(=O)(=O)Nc2ccc(Cl)c3c(Cl)c[nH]c23)cc1)N1CCNCC1]

### 1. Preparation of 4-bromobenzenesulfinic acid

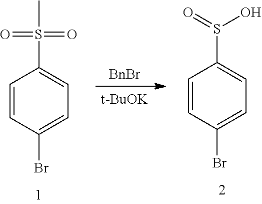

To a solution of 1-bromo-4-methanesulfonylbenzene (5.0 g, 21.27 mmol) in THE (15 mL) were added t-BuOK (6.0 g, 53.16 mmol) and BnBr (4.7 g, 27.65 mmol), which provided a precipitated mixture of potassium benzenesulfinate within minutes of adding benzyl bromide. The mixture was filtered and the filter cake was acidified to pH=2 with 1M aqueous HCl. The mixture was concentrated and purified by reverse phase combi-flash to give 4-bromobenzenesulfinic acid (2.9 g, 62.3%) as a white solid. LCMS (ESI) found: 219 [M−H]⁻.

### 2. Preparation of 4-bromo-N-ethylbenzenesulfinamide

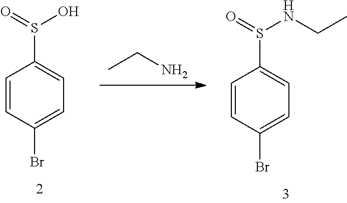

To a solution of 4-bromobenzene-1-sulfinic acid (2.9 g, 13.10 mmol) and DIEA (5.1 g, 39.30 mmol) in DCM (15 mL) was added HATU (7.5 g, 19.65 mmol). The mixture was stirred at rt for 30 min. Ethanamine (1.8 g, 39.30 mmol) was added. The mixture was stirred at rt overnight. The mixture was poured into water (20 mL) and extracted with DCM (25 mL×3). The combined organic layer was dried over MgSO₄, filtered and concentrated to give a residue, which was purified by flash column eluting with DCM/MeOH from 25/1 to 20/1 to give 4-bromo-N-ethylbenzenesulfinamide (1.0 g, 30.8%) as a white solid. LCMS (ESI) found: 248 [M+H]⁺.

### 3. Preparation of 4-bromo-N-ethylbenzenesulfonimidoyl chloride

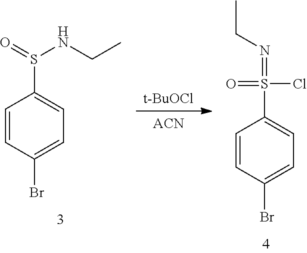

To a solution of 4-bromo-N-ethylbenzene-1-sulfinamide (1.0 g, 4.03 mmol) in CH₃CN (15 mL) was added t-BuOCl (870.5 mg, 8.06 mmol) slowly at 0° C. The mixture was stirred for 1 h at 0° C. until the starting material was consumed completely. The reaction mixture was quenched with aqueous NaHCO₃ (20 mL), diluted with water (20 mL) and extracted with DCM (50 mL×3). The combined organic layer was dried over MgSO₄, filtered and concentrated to give a residue, which was purified by flash to give 4-bromo-N-ethylbenzenesulfonimidoyl chloride (1.1 g, 96.8%) as a white solid. LCMS (ESI) found: 282 [M+H]⁺.

### 4. Preparation of tert-butyl 4-(4-bromo-N-ethylphenylsulfonimidoyl)piperazine-1-carboxy late

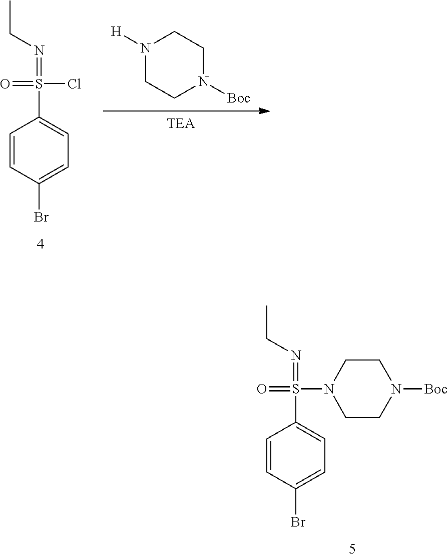

To a solution of 4-bromobenzene-1-sulfinic acid (1.1 g, 3.90 mmol) and TEA (1.2 g, 11.70 mmol) in DCM (10 mL) was added tert-butyl 4-hydrogeniopiperazine-1-carboxylate (725.4 mg, 3.90 mmol). The mixture was stirred at rt for 1 h until the starting material was consumed completely. The mixture was concentrated to give a residue, which was purified by flash column eluting with DCM/MeOH from 25/1 to 20/1 to give tert-butyl 4-(4-bromo-N-ethylphenylsulfonimidoyl) piperazine-1-carboxylate (1.3 g, 77.2%) as a white solid. LCMS (ESI) found: 432 [M+H]⁺.

### 5. Preparation of 1-(4-bromo-N-ethylphenylsulfonimidoyl)piperazine

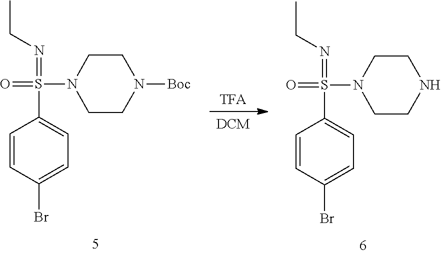

To a solution of tert-butyl 4-(4-bromo-N-ethylphenylsulfonimidoyl) piperazine-1-carboxylate (1.3 g, 3.00 mmol) in DCM (5 mL) was added TFA (2 mL). The mixture was stirred for 1 h until the starting material was consumed completely. The reaction mixture was concentrated to give a crude 1-(4-bromo-N-ethylphenylsulfonimidoyl)piperazine (TFA salt) (1.3 g) as a white solid. LCMS (ESI) found: 332 [M+H]⁺.

### 6. Preparation of 1-(4-(4-bromo-N-ethylphenylsulfonimidoyl)piperazin-1-yl)-2,2,2-trifluoroethan-1-one

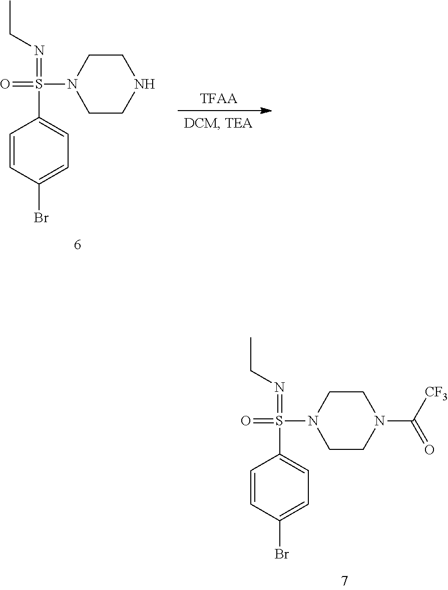 [SMILES: CCN=S(=O)(c1ccc(Br)cc1)N1CCNCC1 ; "TFAA" ; "DCM, TEA" ; CCN=S(=O)(c1ccc(Br)cc1)N1CCN(C(=O)C(F)(F)F)CC1]

To a solution of 1-(4-bromo-N-ethylphenylsulfonimidoyl)piperazine (1.0 g, 3.00 mmol) and TEA (909.0 mg, 9.00 mmol) in DCM (10 mL) was added TFAA (756.0 mg, 3.60 mmol) at 0° C. The mixture was stirred for 1 h at 0° C. until the starting material was consumed completely. The reaction mixture was quenched with aqueous NaHCO₃ (20 mL), diluted with water (20 mL) and extracted with DCM (20 mL×3). The combined organic layer was dried over MgSO₄, filtered and concentrated to give a residue, which was purified by flash eluting with PE/EA from 25/1 to 20/1 to give 1-(4-(4-bromo-N-ethylphenylsulfonimidoyl) piperazin-1-yl)-2,2,2-trifluoroethan-1-one (1.2 g, 93.5%) as a yellow solid. LCMS (ESI) found: 428 [M+H]⁺.

### 7. Preparation of 1-(4-(4-(benzylthio)-N-ethylphenylsulfonimidoyl)piperazin-1-yl)-2,2,2-trifluoroethan-1-one

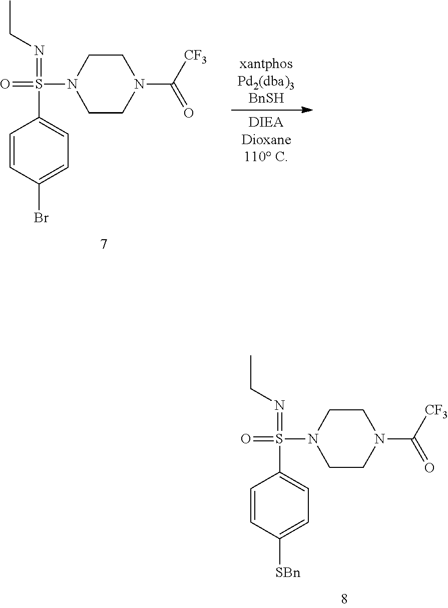

To a solution of 1-(4-(4-bromo-N-ethylphenylsulfonimidoyl)piperazin-1-yl)-2,2,2-trifluoroethan-1-one (1.2 g, 2.80 mmol) and BnSH (347.2 mg, 2.80 mmol) in Dioxane (100 mL), were added DIEA (108.4 mg, 0.84 mmol), Pd₂(dba)₃ (256.5 mg, 0.28 mmol) and Xantphos (323.7 mg, 0.56 mmol). The mixture was evaporated and backfilled with N₂ for three times. The mixture was stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give 1-(4-(4-(benzylthio)-N-ethylphenylsulfonimidoyl)piperazin-1-yl)-2,2,2-trifluoroethan-1-one (900 mg, 68.1%) as a yellow solid. LCMS (ESI) found: 472 [M+H]⁺.

### 8. Preparation of 4-(N-ethyl-4-(2,2,2-trifluoroacetyl)piperazine-1-sulfonimidoyl)benzenesulfonyl chloride

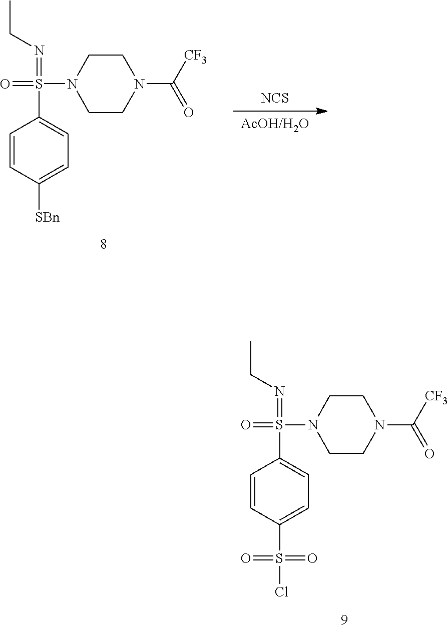

To a solution of 1-(4-(4-(benzylthio)-N-ethylphenylsulfonimidoyl)piperazin-1-yl)-2,2,2-trifluoroethan-1-one (500 mg, 1.06 mmol) in AcOH/H₂O (9/1, 4 mL) was added NCS (596.6 mg, 4.45 mmol). The mixture was stirred at rt for 2 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford 4-(N-ethyl-4-(2,2,2-trifluoroacetyl) piperazine-1-sulfonimidoyl)benzenesulfonylchloride (400.0 mg, 84.2%) as a white solid. LCMS (ESI) found: 448 [M+H]⁺.

### 9. Preparation of N-(3,4-dichloro-1H-indol-7-yl)-4-(N-ethyl-4-(2,2,2-trifluoroacetyl)piperazine-1-sulfonimidoyl)benzenesulfonamide

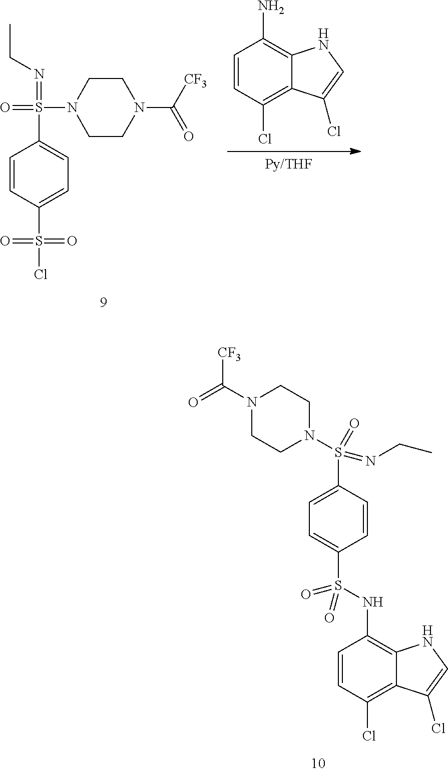

To a solution of 3,4-dichloro-1H-indol-7-amine (25 mg, 0.12 mmol) and Py (29.6 mg, 0.37 mmol) in THE (10 mL) was added 4-(N-ethyl-4-(2,2,2-trifluoroacetyl)piperazine-1-sulfon imidoyl)benzenesulfonyl chloride (53.8 mg, 0.12 mmol). The mixture was stirred at rt overnight. The mixture was concentrated to give a residue, which was purified by prep-TLC to give N-(3,4-dichloro-1H-indol-7-yl)-4-(N-ethyl-4-(2,2,2-trifluoroacetyl)piperazine-1-sulfonimidoyl)benzenesulfonamide (40.0 mg, 54.5%) as a white solid. LCMS (ESI) found: 612 [M+H]⁺.

### 10. Preparation of N-(3,4-dichloro-1H-indol-7-yl)-4-(N-ethylpiperazine-1-sulfonimidoyl) benzenesulfonamide

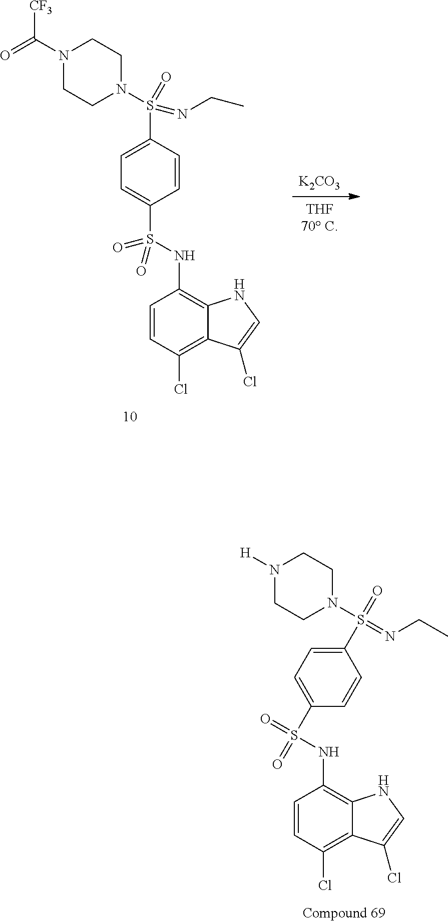 [SMILES: CCN=S(=O)(c1ccc(S(=O)(=O)Nc2ccc(Cl)c3c(Cl)c[nH]c23)cc1)N1CCN(C(=O)C(F)(F)F)CC1 ; "K2CO3" ; "THF70° C." ; CCN=S(=O)(c1ccc(S(=O)(=O)Nc2ccc(Cl)c3c(Cl)c[nH]c23)cc1)N1CCNCC1]

To a solution of N-(3,4-dichloro-1H-indol-7-yl)-4-(N-ethyl-4-(2,2,2-trifluoroacetyl) piperazine-1-sulfonimidoyl)benzenesulfonamide (40.0 mg, 0.065 mmol) in THF/H₂O (4/1, 10 mL) was added K₂CO₃ (269.1 mg, 1.95 mmol). The mixture was stirred at 70° C. for 2 h. The reaction mixture was filtered and the filtrate was concentrated to give a crude product, which was purified by prep-HPLC to give. N-(3,4-dichloro-1H-indol-7-yl)-4-(N-ethyl piperazine-1-sulfonimidoyl)benzenesulfonamide (HCOOH salt) (Compound 69, 11.2 mg, 33.8%) as a white solid. LCMS (ESI) found: 516 [M+H]⁺. ¹H NMR (400 MHz, DMSO-d6) δ 11.35 (s, 1H), 8.16 (s, 0.58H), 7.91 (d, J=8.5 Hz, 2H), 7.82 (d, J=8.5 Hz, 2H), 7.36 (s, 1H), 6.78 (s, 1H), 6.65 (s, 1H), 3.15-3.12 (m, 1H), 3.08-3.04 (m, 1H), 2.90 (s, 4H), 2.83 (s, 4H), 1.15 (t, J=7.2 Hz, 3H).

### Example 37: Preparation of N-(3,4-dichloro-1H-indol-7-yl)-4-(difluoro(piperidin-4-yl)methyl)benzene sulfonamide; Compound 70

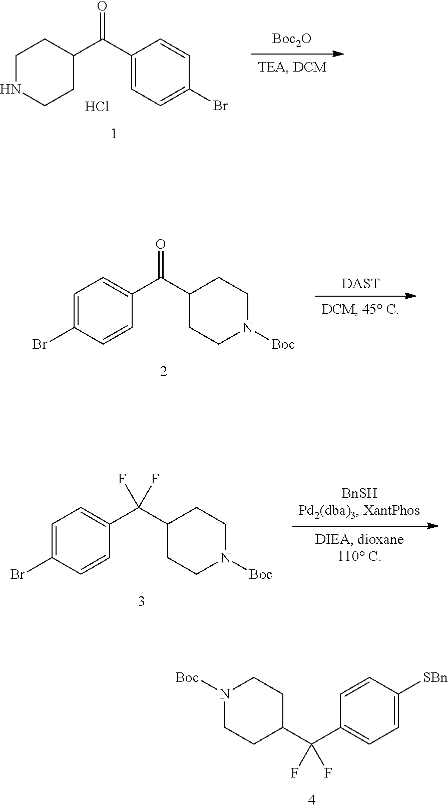

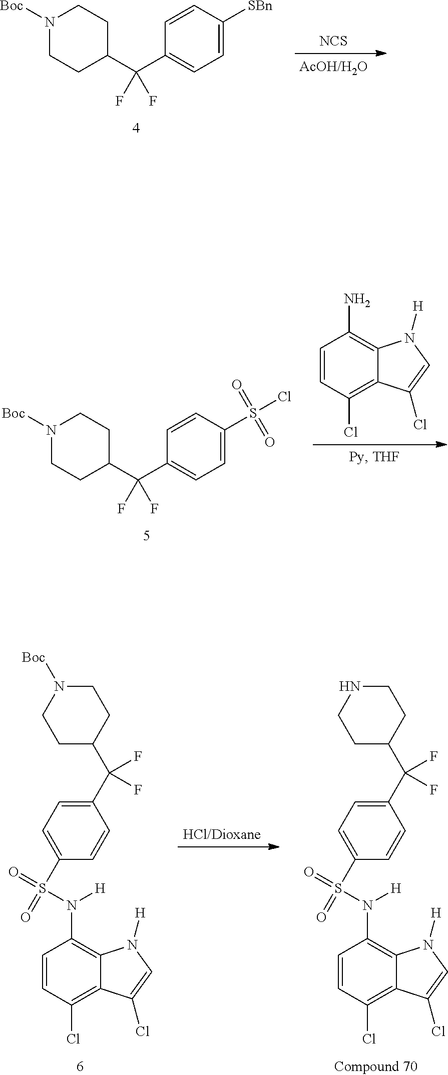

### 1. Preparation of tert-butyl 4-(4-bromobenzoyl)piperidine-1-carboxylate

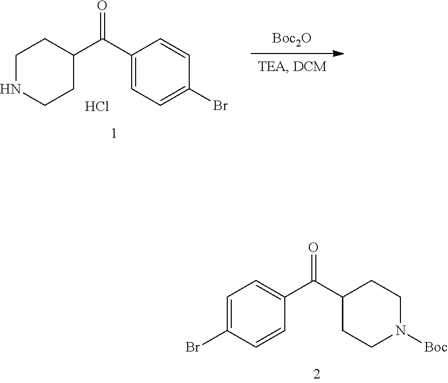 [SMILES: "Boc2O" ; O=C(c1ccc(Br)cc1)C1CCNCC1 ; "TEA, DCM" ; Cl ; CC(C)(C)OC(=O)N1CCC(C(=O)c2ccc(Br)cc2)CC1]

To a mixture of (4-bromophenyl)(piperidin-4-yl)methanone hydrochloride (300.0 mg, 0.99 mmol) and TEA (339.0 mg, 3.36 mmol) in DCM (10 mL) was added Boc₂O (268.3 mg, 1.23 mmol). The mixture was stirred for 2 h at rt. The mixture was concentrated and purified by flash (eluting with 25% EA in PE) to afford tert-butyl 4-(4-bromobenzoyl)piperidine-1-carboxylate (300.0 mg, 82.6% yield). LCMS (ESI): 312 [M+H-56]⁺.

### 2. Preparation of tert-butyl 4-((4-bromophenyl)difluoromethyl)piperidine-1-carboxylate

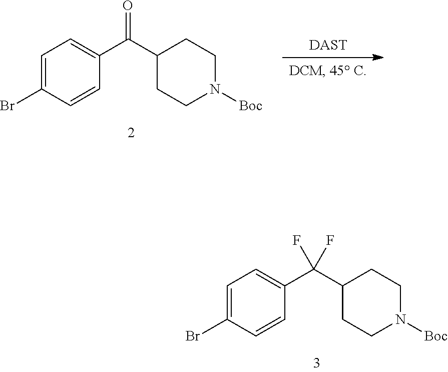 [SMILES: CC(C)(C)OC(=O)N1CCC(C(=O)c2ccc(Br)cc2)CC1 ; "DAST" ; "DCM, 45° C." ; CC(C)(C)OC(=O)N1CCC(C(F)(F)c2ccc(Br)cc2)CC1]

To a mixture of tert-butyl 4-(4-bromobenzoyl)piperidine-1-carboxylate (250.0 mg, 0.68 mmol) in DCM (1 mL) was added DAST (3830.0 mg, 23.79 mmol) at rt. The mixture was heated at 45° C. for 4 days in a sealed tube. The mixture was cooled to rt and poured into ice water (50 mL) and extracted with EA (50 mL×3). The combined organic layer was concentrated and purified by flash (eluting with 8% DCM in PE) to afford tert-butyl 4-((4-bromophenyl)difluoromethyl)piperidine-1-carboxylate (220.0 mg, 83.0%). LCMS (ESI): 334 [M+H-56]⁺.

### 3. Preparation of tert-butyl 4-((4-(benzylthio)phenyl)difluoromethyl)piperidine-1-carboxylate

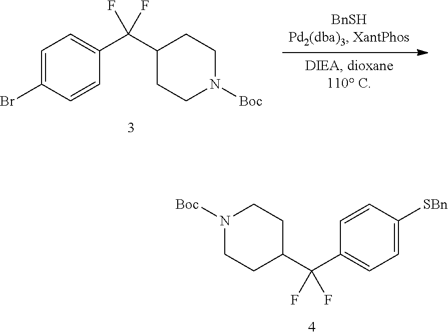

To a mixture of tert-butyl 4-((4-bromophenyl)difluoromethyl)piperidine-1-carboxylate (50.0 mg, 0.13 mmol), BnSH (32.0 mg, 0.26 mmol), DIPEA (50.0 mg, 0.39 mmol) in dioxane (5 mL) were added Pd₂(dba)₃ (10.0 mg, 0.01 mmol) and XantPhos (22.0 mg, 0.04 mmol). The mixture was evaporated and backfilled with N₂ for three times. The mixture was stirred at 110° C. under N₂ atmosphere for 3 h until the starting material was consumed completely. The mixture was cooled to room temperature and concentrated in vacuum to remove most of solvent. The residue was diluted with water (20 mL) and extracted with EA (20 mL×3). The combined organic layer was dried over MgSO₄, filtered and concentrated to give a crude product, which was purified by column chromatography on silica gel eluting with PE/EA from 30/1 to 5/1 to give tert-butyl 4-((4-bromophenyl)difluoromethyl)piperidine-1-carboxylate (40.0 mg, 72.0%). LCMS (ESI): 334 [M+H-100]⁺.

### 4. Preparation of tert-butyl 4-((4-(chlorosulfonyl)phenyl)difluoromethyl)piperidine-1-carboxylate

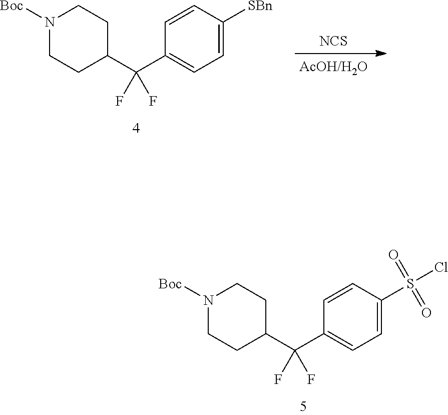

To a mixture of tert-butyl 4-((4-bromophenyl)difluoromethyl)piperidine-1-carboxylate (780.0 mg, 1.80 mmol) in DCM (10 mL) and AcOH/H₂O (9/1, 4 mL) was added NCS (965.0 mg, 7.201 mmol) at rt. The mixture was stirred at rt for 2 h. The reaction was quenched with aqueous Na₂S₂O₃ (15 mL) and extracted with DCM (15 mL×3). The combined organic layer was concentrated to give a crude product, which was purified by column chromatography on silica gel eluting with PE/EA from 15/1 to 5/1 to afford tert-butyl 4-((4-(chlorosulfonyl)phenyl)difluoromethyl)piperidine-1-carboxylate (500.0 mg, 67.8%). LCMS (ESI): 410 [M+H]⁺

### 5. Preparation of tert-butyl 4-((4-(N-(3,4-dichloro-1H-indol-7-yl)sulfamoyl)phenyl)difluoro methyl)piperidine-1-carboxylate

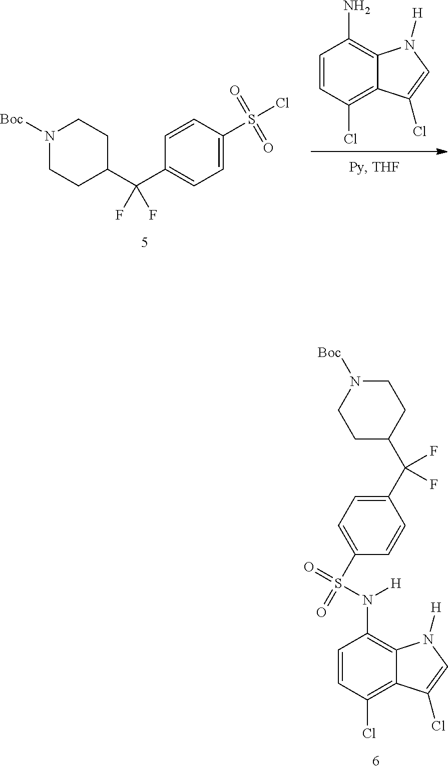

To a mixture of 3,4-dichloro-1H-indol-7-amine (140.0 mg, 0.70 mmol) and Py (0.2 mL) in THE (2 mL) was added tert-butyl 4-((4-(chlorosulfonyl)phenyl)difluoromethyl)piperidine-1-carboxylate (286.0 mg, 0.70 mmol). The mixture was stirred at rt for 3 h. The mixture was concentrated and purified by flash (eluting with 50% EA in PE) to afford tert-butyl 4-((4-(N-(3,4-dichloro-1H-indol-7-yl)sulfamoyl)phenyl)difluoromethyl)piperidine-1-carboxylate (300.0 mg, 74.7%). LCMS (ESI): 574 [M+H]⁺

### 6. Preparation of N-(3,4-dichloro-1H-indol-7-yl)-4-(difluoro(piperidin-4-yl)methyl)benzene sulfonamide

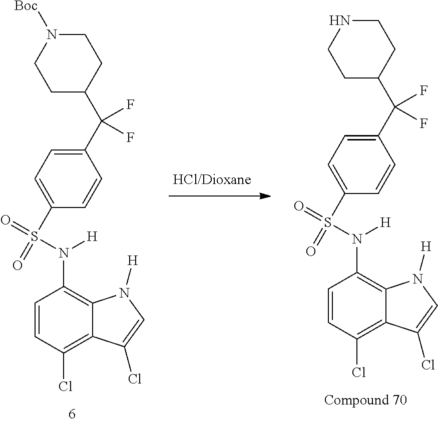 [SMILES: CC(C)(C)OC(=O)N1CCC(C(F)(F)c2ccc(S(=O)(=O)Nc3ccc(Cl)c4c(Cl)c[nH]c34)cc2)CC1 ; O=S(=O)(Nc1ccc(Cl)c2c(Cl)c[nH]c12)c1ccc(C(F)(F)C2CCNCC2)cc1]

A mixture of tert-butyl 4-((4-(N-(3,4-dichloro-1H-indol-7-yl)sulfamoyl)phenyl)difluoromethyl) piperidine-1-carboxylate (200.0 mg, 0.35 mmol) in HCl/dioxane (5 mL) was stirred at rt for 2 h. The mixture was concentrated and basified with aqueous NaHCO₃ (20 mL) and extracted with DCM (20 mL×3). The combined organic layer was concentrated and purified by prep-HPLC to afford N-(3,4-dichloro-1H-indol-7-yl)-4-(difluoro(piperidin-4-yl)methyl) benzenesulfonamide (HCOOH salt) (Compound 70, 31.2 mg, 17.5%). LCMS (ESI): 474 [M+H]⁺. ¹H NMR (400 MHz, DMSO-d6) δ 11.28 (s, 1H), 8.20 (s, 0.83H), 7.84 (d, J=8.3 Hz, 2H), 7.46 (d, J=8.4 Hz, 2H), 7.22 (s, 1H), 6.73-6.66 (m, 2H), 3.21 (s, 1H), 2.77 (t, J=11.7 Hz, 2H), 2.69-2.54 (m, 1H), 2.48-2.39 (m, 1H), 1.70-1.64 (m, 2H), 1.48-1.40 (m, 2H), 1.23 (s, 1H).

### Example 38: Preparation of N-(4-(N-(3,4-dichloro-1H-indol-7-yl)sulfamoyl)phenyl)-N-propylacetamide; Compound 71

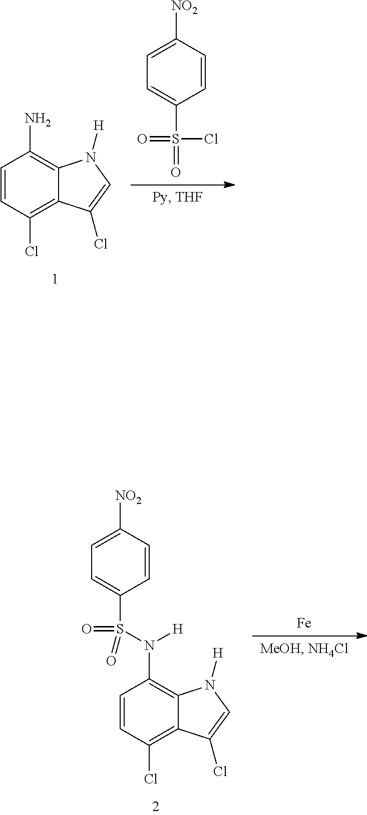

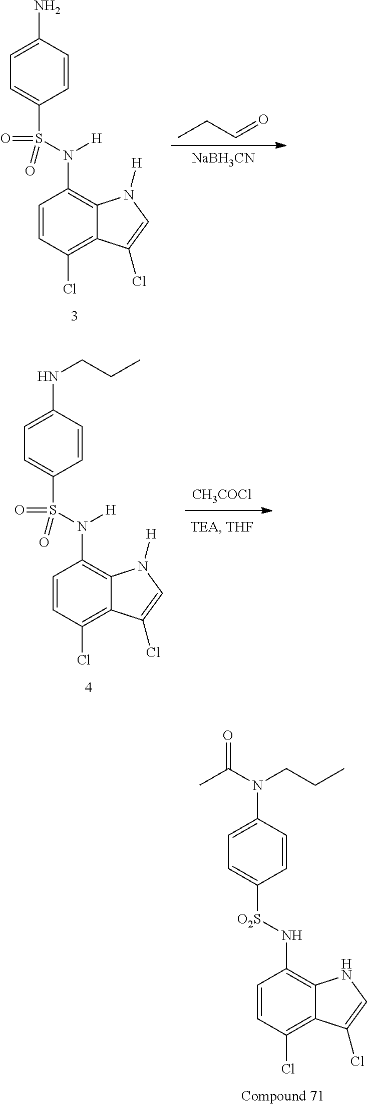

### 1. Preparation of N-(3,4-dichloro-1H-indol-7-yl)-4-((2-methyl-2-azaspiro[3.3]heptan-6-yl)oxy)benzenesulfonamide

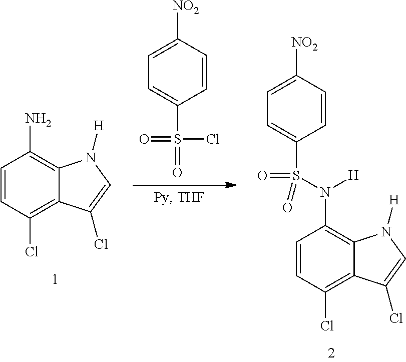

To a solution of 3,4-dichloro-1H-indol-7-amine (30.2 mg, 0.15 mmol) and pyridine (35.6 mg, 0.45 mmol) in THF (2 mL) was added 4-nitrobenzenesulfonyl chloride (33.3 mg, 0.15 mmol). The mixture was stirred at rt for 4 h. The mixture was concentrated and purified by column chromatography eluting with PE/EA from 20/1 to 5/1 to give N-(3,4-dichloro-1H-indol-7-yl)-4-nitrobenzenesulfonamide (54.8 mg, 94.3%) as a yellow solid. LCMS (ESI) found: 386 [M+H]⁺.

### 2. Preparation of 4-amino-N-(3,4-dichloro-1H-indol-7-yl)benzenesulfonamide

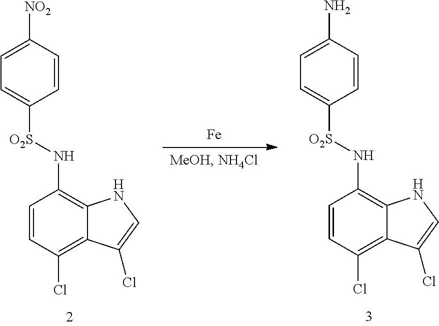

To a solution of N-(3,4-dichloro-1H-indol-7-yl)-4-nitrobenzenesulfonamide (50.0 mg, 0.13 mmol) in MeOH/NH₄Cl=1/1 (3 mL) was added Fe (37.1 mg, 0.65 mmol). The mixture was stirred for 1 h at 80° C. TLC showed SM has consumed. The reaction mixture was diluted with water (20 mL) and extracted with EA (30 mL×3). The organic layer was washed with water (20 mL), brine (20 mL), dried over Na₂SO₄ and concentrated under reduced pressure to afford the crude product, which was purified by column chromatography on silica gel eluted with DCM/MEOH (pure DCM to 10/1) to give 4-amino-N-(3,4-dichloro-1H-indol-7-yl)benzenesulfonamide (40.3 mg, 87.4%) as a white solid. LCMS (ESI) found: 356 [M+H]⁺.

### 3. Preparation of N-(3,4-dichloro-1H-indol-7-yl)-4-(propylamino)benzenesulfonamide

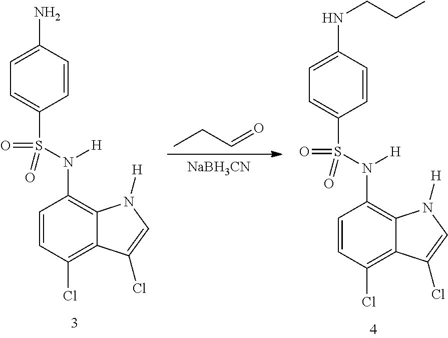

To a solution of 4-amino-N-(3,4-dichloro-1H-indol-7-yl)benzenesulfonamide (40.0 mg, 0.11 mmol) in MeOH (5 mL) were added a drop of AcOH and propanal (19.6 mg, 0.33 mmol). The mixture was stirred at rt for 30 min. NaBH₃CN (11.4 mg, 0.33 mmol) was added. The mixture was continued to stirred at rt overnight. The mixture was concentrated and purified by flash eluting with PE/EA from 95/5 to 80/20 to give N-(3,4-dichloro-1-hydrogenio-1H-indol-7-yl)-4-(propylamino)benzene-1-hydrogeniosulfonamide (39.0 mg, 87.2%) as an off-white solid. LCMS (ESI) found: 398 [M+H]⁺.

### 4. Preparation of N-(4-(N-(3,4-dichloro-1H-indol-7-yl)sulfamoyl)phenyl)-N-propylacetamide

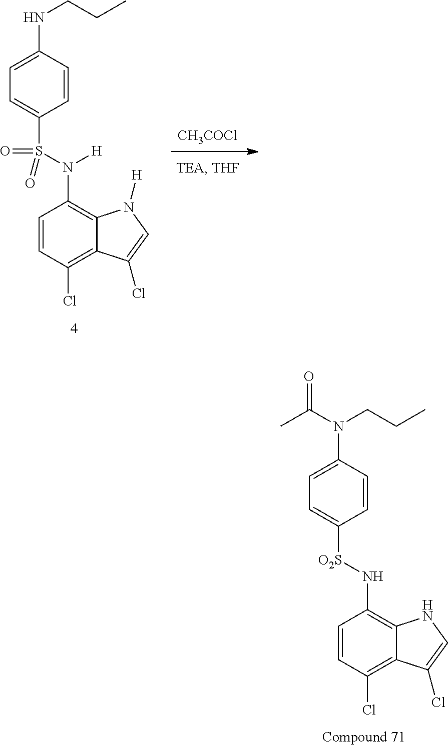 [SMILES: CCCNc1ccc(S(=O)(=O)Nc2ccc(Cl)c3c(Cl)c[nH]c23)cc1 ; "CH3COCl" ; CCCN(C(C)=O)c1ccc(S(=O)(=O)Nc2ccc(Cl)c3c(Cl)c[nH]c23)cc1]

To a solution of N-(3,4-dichloro-1-hydrogenio-1H-indol-7-yl)-4-(propylamino)benzene-1-hydrogeniosulfonamide (20.0 mg, 0.05 mmol) in THF (1 mL) were added TEA (10.1 mg, 0.10 mmol) and CH₃COCl (0.5 mL, 0.05 mmol) (0.1 M in THF). The mixture was stirred at −20° C. for 2 h. The mixture was quenched with water (15 mL) and extracted with DCM (20 mL×3). The combined organic layer was concentrated and purified by reversed phase flash eluting with CH₃OH/H₂O (0.1% NH₄0H) from 2/98 to 98/2 to give N-(4-(N-(3,4-dichloro-1H-indol-7-yl)sulfamoyl)phenyl)-N-propylacetamide (Compound 71, 4.3 mg, 19.5%) as a white solid. LCMS (ESI) found: 440 [M+H]⁺. ¹H NMR (400 MHz, DMSO-d6) δ 11.32 (s, 1H), 10.00 (s, 1H), 7.75-7.62 (m, 2H), 7.52 (d, J=2.8 Hz, 1H), 7.44 (d, J=8.6 Hz, 2H), 6.94 (d, J=8.2 Hz, 1H), 6.67 (d, J=8.2 Hz, 1H), 3.61-3.55 (t, J=7.4 Hz, 2H), 1.75 (s, 3H), 1.36-1.27 (m, 2H), 0.77 (t, J=7.4 Hz, 3H).

### Example 39: Preparation of 4-(N-(3,4-dichloro-1H-indol-7-yl)sulfamoyl)benzenesulfonyl fluoride Compound 72

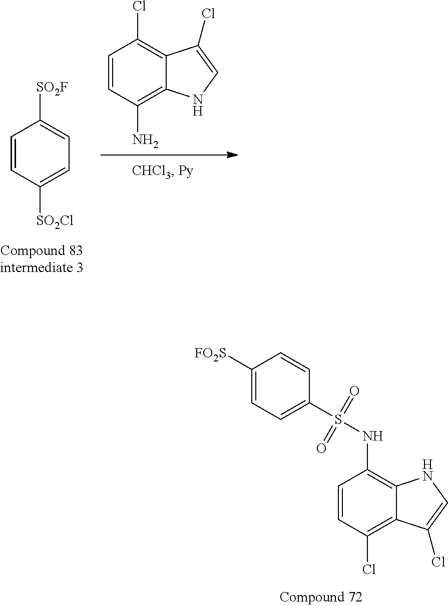

### 1. Preparation of 4-(N-(3,4-dichloro-1H-indol-7-yl)sulfamoyl)benzenesulfonyl fluoride

To a solution of 3,4-dichloro-1H-indol-7-amine (50.0 mg, 0.25 mmol) and pyridine (59.2 mg, 0.75 mmol) in CHCl₃ (2 mL) was added 4-(chlorosulfonyl) benzenesulfonyl fluoride (64.7 mg, 0.25 mmol). The reaction mixture was stirred at rt for 2 h. LCMS showed the reaction was completed and the solvent was concentrated under reduced pressure to give a crude product, which was purified by prep-TLC to give 4-(N-(3,4-dichloro-1H-indol-7-yl)sulfamoyl) benzenesulfonyl fluoride (Compound 72, 70.0 mg, yield: 66.2%) as a white solid. LCMS (ESI) found: 423 [M+H]⁺. ¹H NMR (400 MHz, DMSO-d6) δ 11.57 (s, 1H), 10.44 (s, 1H), 8.35 (d, J=8.6 Hz, 2H), 8.07 (d, J=8.5 Hz, 2H), 7.60 (d, J=2.8 Hz, 1H), 6.99 (d, J=8.1 Hz, 1H), 6.62 (d, J=8.1 Hz, 1H).

### Example 40: Preparation of 3-cyano-N-(3,4-dichloro-1H-indol-7-yl)benzenesulfonamide; Compound 74

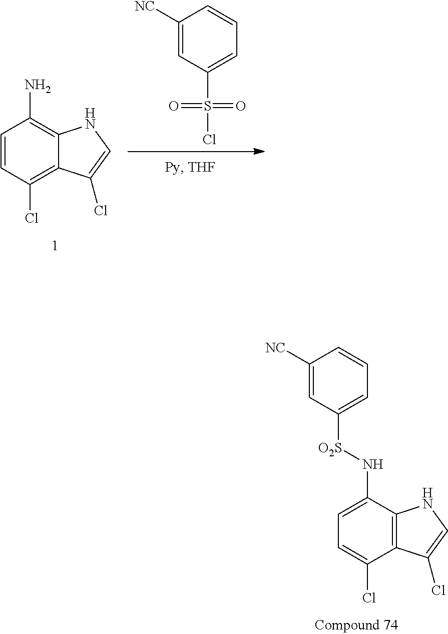 [SMILES: Nc1ccc(Cl)c2c(Cl)c[nH]c12 ; N#Cc1cccc(S(=O)(=O)Cl)c1 ; N#Cc1cccc(S(=O)(=O)Nc2ccc(Cl)c3c(Cl)c[nH]c23)c1]

### 1. Preparation of 3-cyano-N-(3,4-dichloro-1H-indol-7-yl)benzenesulfonamide

To a solution of 3,4-dichloro-1H-indol-7-amine (30.0 mg, 0.15 mol) and pyridine (35.6 mg, 0.45 mmol) in THF (5 mL) was added 3-cyanobenzenesulfonyl chloride (30.1 mg, 0.15 mmol). The mixture was stirred at rt for 3 h. The mixture was concentrated to give a crude product, which was purified by prep-HPLC to give 3-cyano-N-(3,4-dichloro-1H-indol-7-yl)benzene sulfonamide (Compound 74, 38.2 mg, yield: 69.5%) as a white solid. LCMS (ESI) found: 366 [M+H]⁺. ¹H NMR (400 MHz, DMSO-d6) δ 11.50 (s, 1H), 10.19 (s, 1H), 8.16 (s, 1H), 8.11 (d, J=7.9 Hz, 1H), 7.95 (d, J=8.1 Hz, 1H), 7.74 (t, J=7.8 Hz, 1H), 7.56 (s, 1H), 6.96 (d, J=8.2 Hz, 1H), 6.62 (d, J=8.2 Hz, 1H).

### Example 41: Preparation of 4-bromo-N-(3,4-dichloro-1H-indol-7-yl)benzenesulfonamide Compound 75

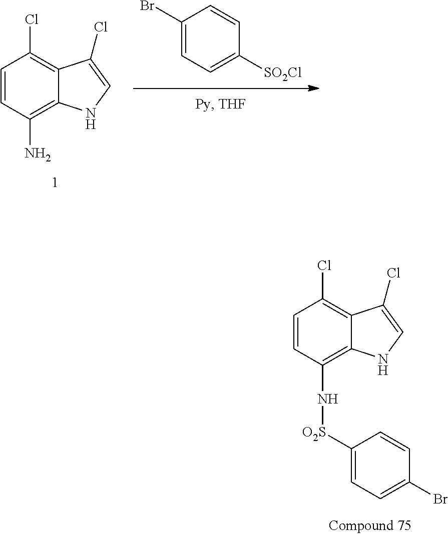 [SMILES: Nc1ccc(Cl)c2c(Cl)c[nH]c12 ; O=S(=O)(Cl)c1ccc(Br)cc1 ; O=S(=O)(Nc1ccc(Cl)c2c(Cl)c[nH]c12)c1ccc(Br)cc1]

### Preparation of 4-bromo-N-(3,4-dichloro-1H-indol-7-yl)benzenesulfonamide

To a solution of 3,4-dichloro-1H-indol-7-amine (360.0 mg, 1.79 mmol) and pyridine (0.3 mL) in THF (10 mL), was added 4-bromobenzenesulfonyl chloride (457.5 mg, 1.79 mmol). The mixture was stirred at rt for 2 h. The mixture was concentrated and purified by prep-HPLC to give 4-bromo-N-(3,4-dichloro-1H-indol-7-yl)benzenesulfonamide (Compound 75, 400.0 mg, 53.2%) as a white solid. LCMS (ESI): 419 [M+H]⁺. ¹HNMR (400 MHz, DMSO-d6) δ 11.44 (s, 1H), 10.09 (s, 1H), 7.80-7.72 (m, 2H), 7.65-7.60 (m, 2H), 7.57 (d, J=2.9 Hz, 1H), 6.96 (d, J=8.2 Hz, 1H), 6.64 (d, J=8.2 Hz, 1H).

### Example 42: Preparation of 3-bromo-N-(3,4-dichloro-1H-indol-7-yl)benzenesulfonamide; Compound 76

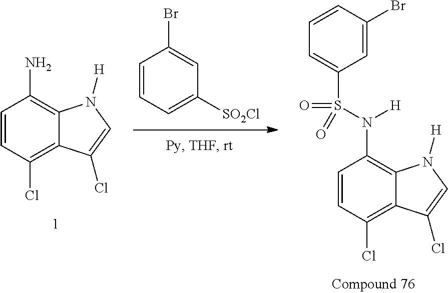

### 1. Preparation of N-(3,4-dichloro-1H-indol-7-yl)-4-((2-methyl-2-azaspiro[3.3]heptan-6-yl)oxy)benzenesulfonamide

To a solution of 3,4-dichloro-1H-indol-7-amine (20.0 mg, 0.1 mmol) and pyridine (23.6 mg, 0.30 mmol) in THF (2 mL) was added 3-bromobenzenesulfonyl chloride (254.2 mg, 0.1 mmol) at rt. The mixture was stirred at rt overnight. The mixture was concentrated and purified by prep-TLC to give 3-bromo-N-(3,4-dichloro-1H-indol-7-yl)benzenesulfonamide (Compound 76, 25.5 mg, 61%) as a white solid. LCMS (ESI) found: 419 [M+H]⁺. ¹H NMR (400 MHz, DMSO-d6) δ 11.50 (s, 1H), 10.10 (s, 1H), 7.91-7.78 (m, 2H), 7.74-7.63 (m, 1H), 7.58 (d, J=2.9 Hz, 1H), 7.49 (t, J=7.9 Hz, 1H), 6.97 (d, J=8.2 Hz, 1H), 6.62 (d, J=8.2 Hz, 1H).

### Example 43: Preparation of N¹-(3-cyano-4-methyl-1H-indol-7-yl)-N⁴-methyl-N⁴-(piperidin-4-yl) benzene-1,4-disulfonamide; Compound 78)

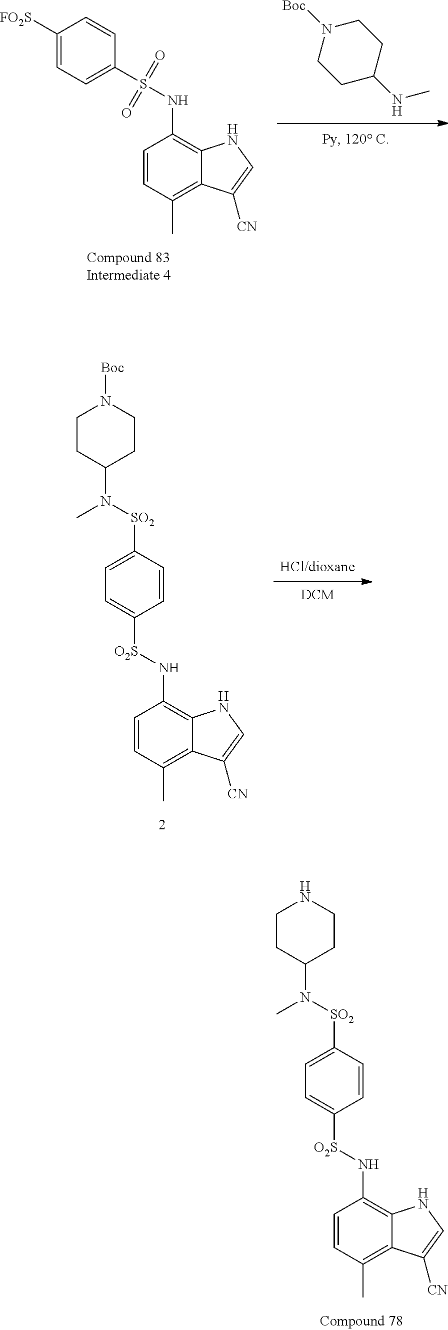

### 1. Preparation of tert-butyl 4-((4-(N-(3-cyano-4-methyl-1H-indol-7-yl)sulfamoyl)-N-methyl phenyl)sulfonamido)piperidine-1-carboxylate

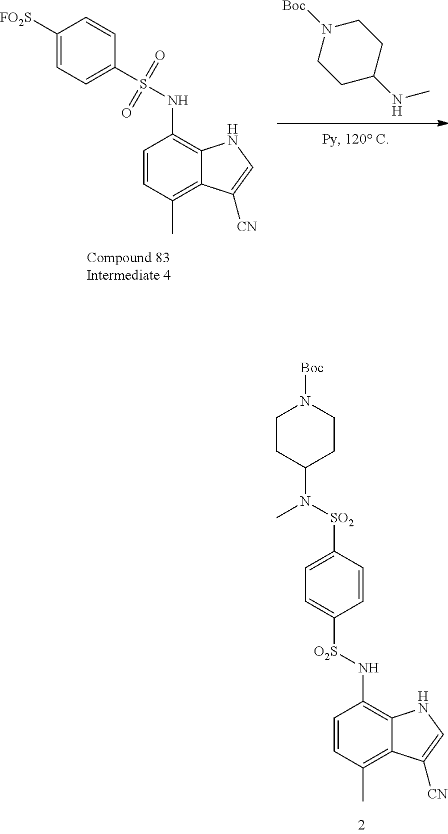

To a solution of 4-(methylamino)piperidine-1-carboxylate (20.0 mg, 0.09 mmol) in pyridine (5 mL) was added tert-butyl 4-(N-(3-cyano-4-methyl-1H-indol-7-yl)sulfamoyl)benzenesulfonyl fluoride (36.8 mg, 0.09 mmol). The mixture was stirred at 120° C. overnight. The mixture was concentrated to afford a crude product, which was purified by column chromatography on silica gel eluting with PE/EA to give tert-butyl 4-((4-(N-(3-cyano-4-methyl-1H-indol-7-yl) sulfamoyl)-N-methylphenyl)sulfonamido)piperidine-1-carboxylate (15.0 mg, yield 28.4%) as colorless oil. LCMS (ESI) found: 588 [M+H]⁺.

### 2. Preparation of N¹-(3-cyano-4-methyl-1H-indol-7-yl)-N⁴-methyl-N⁴-(piperidin-4-yl) benzene-1,4-disulfonamide

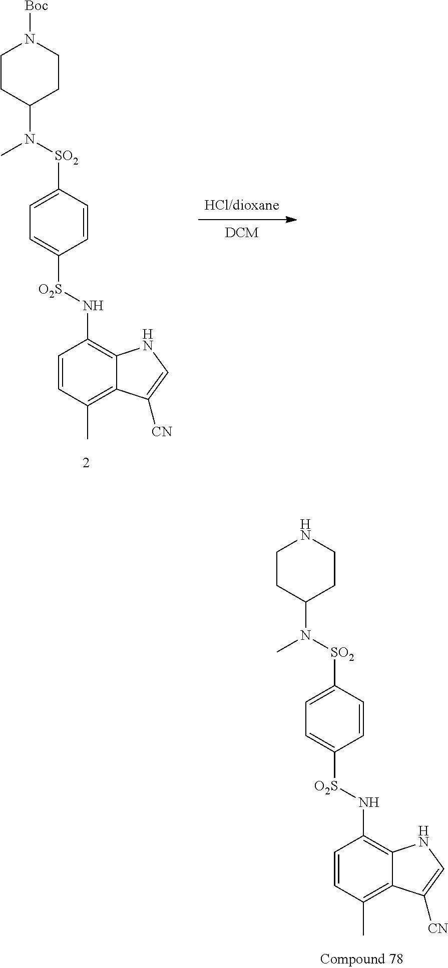

To a solution of tert-butyl 4-((4-(N-(3-cyano-4-methyl-1H-indol-7-yl)sulfamoyl)-N-methylphenyl)sulfonamido)piperidine-1-carboxylate (15.0 mg, 0.025 mmo) in DCM (3 mL) was added HCl in dioxane (4 M/L, 1 mL). The mixture was stirred at room temperature for 30 min. The mixture was concentrated. The residue was basified with 10% aqueous NaHCO₃ (20 mL) and extracted with DCM (20 mL×3). The combined organic layer was concentrated and the residue was purified by prep-HPLC to give N¹-(3-cyano-4-methyl-1H-indol-7-yl)-N⁴-methyl-N⁴-(piperidin-4-yl)benzene-1,4-disulfonamide as white solid (Compound 78, 2.0 mg, yield: 16.4%), LCMS (ESI) found: 488 [M+H]⁺. ¹H NMR (400 MHz, CD₃OD) δ 7.97-7.91 (m, 3H), 7.82 (d, J=8.5 Hz, 2H), 6.74 (d, J=8.1 Hz, 1H), 6.42 (d, J=8.0 Hz, 1H), 4.09-4.04 (m, 1H), 3.29-3.25 (m, 2H), 2.99-2.94 (m, 2H), 2.78 (s, 3H), 2.65 (s, 3H), 1.81-1.74 (m, 2H), 1.57-1.50 (m, 2H).

### Example 44: Preparation of N¹-(azetidin-3-yl)-N⁴-(3-cyano-4-methyl-1H-indol-7-yl)-N¹-methylbenzene-1,4-disulfonamide; Compound 79

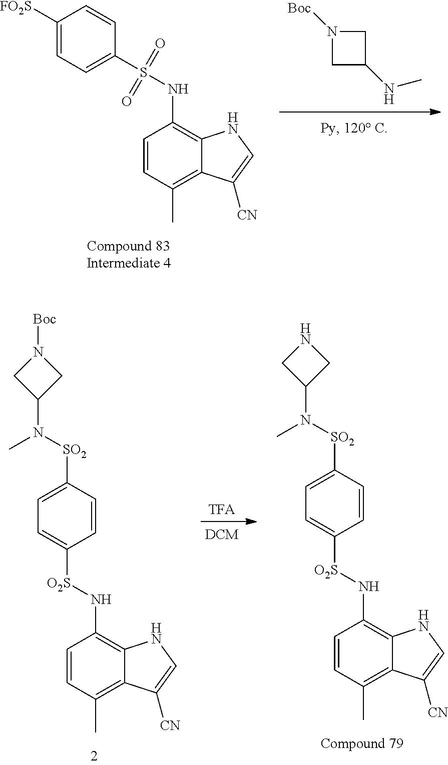

### 1. Preparation of tert-butyl 3-((4-(N-(3-cyano-4-methyl-1H-indol-7-yl)sulfamoyl)-N-methyl phenyl)sulfonamido)azetidine-1-carboxylate

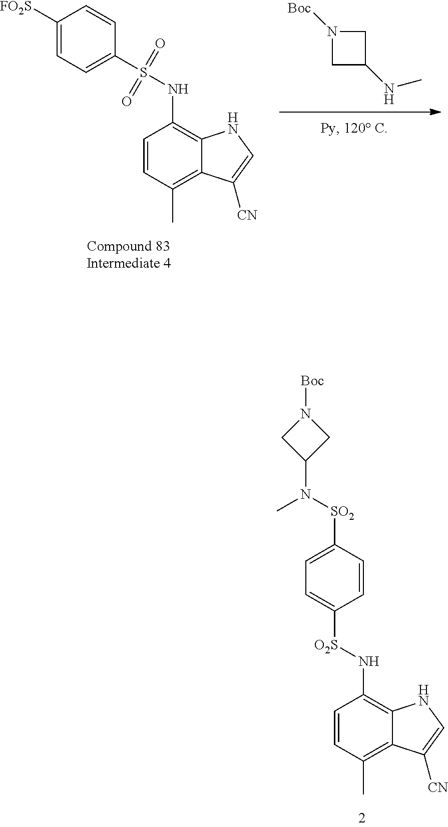

To a solution of 4-(N-(3-cyano-4-methyl-1H-indol-7-yl)sulfamoyl)benzenesulfonyl fluoride (50.0 mg, 0.12 mmol) in pyridine (2 mL) was added tert-butyl 3-(methylamino)azetidine-1-carboxylate (26.0 mg, 0.14 mmol). The mixture was stirred for 18 h at 120° C. The mixture was concentrated to afford a crude product, which was purified by column chromatography on silica gel eluting with PE/EA=5/1 to give tert-butyl 3-((4-(N-(3-cyano-4-methyl-1H-indol-7-yl) sulfamoyl)-N-methylphenyl)sulfonamido)azetidine-1-carboxylate (21.3 mg, 0.04 mmol, yield: 21.69%) as a white solid. LCMS (ESI) found: 560 [M+H]⁺.

### 2. Preparation of N¹-(azetidin-3-yl)-N₄-(3-cyano-4-methyl-1H-indol-7-yl)-N¹-methylbenzene-1,4-disulfonamide

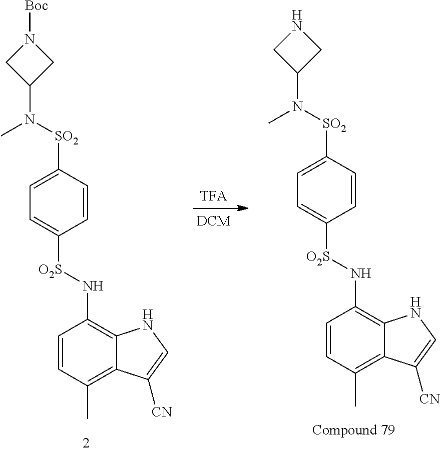

To a solution of tert-butyl 3-((4-(N-(3-cyano-4-methyl-1H-indol-7-yl)sulfamoyl)-N-methyl phenyl)sulfonamido)azetidine-1-carboxylate (21.3 mg, 0.04 mmol) in DCM (2 mL) was added TFA (1 mL). The mixture was stirred at rt for 2 h. The mixture was concentrated. The residue was basified with 10% aqueous NaHCO₃ (20 mL) and extracted with DCM (20 mL×3). The combined organic layer was concentrated and the residue was purified by prep-HPLC to give N¹-(azetidin-3-yl)-N⁴-(3-cyano-4-methyl-1H-indol-7-yl)-N¹-methyl benzene-1,4-disulfonamide (Compound 79, 5.9 mg, yield: 32.1%) as a white solid. LCMS (ESI) found: 460 [M+H]⁺. ¹H NMR (400 MHz, DMSO-d6) δ 12.02 (s, 1H) 8.18 (s, 1H), 7.92-7.89 (m, 4H), 6.78 (d, J=7.9 Hz, 1H), 6.48 (d, J=7.7 Hz, 1H), 4.55-4.48 (m, 1H), 4.05-4.00 (m, 2H), 3.97-3.93 (m, 2H), 2.74 (s, 3H), 2.58 (s, 3H).

### Example 45: Preparation of N-(3-cyano-4-methyl-1H-indol-7-yl)-4-((3-(dimethylamino)pyrrolidine-1-yl)sulfonyl)benzenesulfonamide; Compound 80

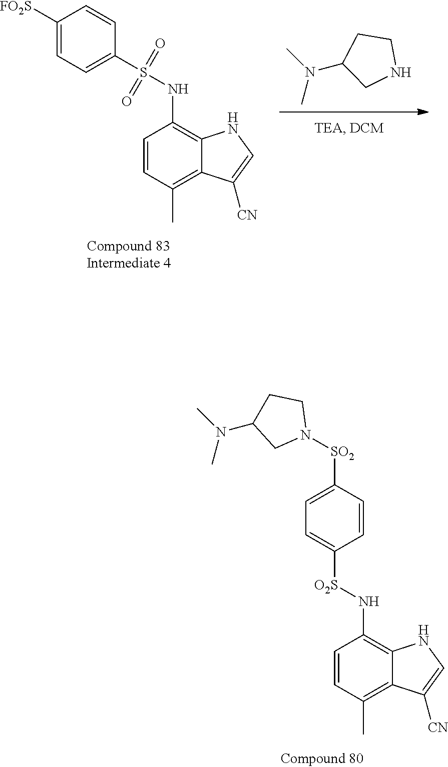

### 1. Preparation of N-(3-cyano-4-methyl-1H-indol-7-yl)-4-((3-(dimethylamino)pyrrolidine-1-yl)sulfonyl)benzenesulfonamide

To a solution of 4-(N-(3-cyano-4-methyl-1H-indol-7-yl)sulfamoyl)benzenesulfonyl fluoride (25.0 mg, 0.06 mmol) and TEA (19.2 mg, 0.19 mmol) in DCM (2 mL) was added N,N-dimethylpyrrolidin-3-amine (7.2 mg, 0.07 mmol). The mixture was stirred for 18 h at room temperature. The mixture was concentrated to afford a crude product, which was purified by column chromatography on silica gel eluting with PE/EA to give N-(3-cyano-4-methyl-1H-indol-7-yl)-4-((3-(dimethylamino)pyrrolidin-1-yl)sulfonyl)benzenesulfonamide (Compound 80, 15.8 mg, yield: 54.0%) as a white solid. LCMS (ESI) found: 488 [M+H]⁺. ¹H NMR (400 MHz, DMSO-d6) δ 11.95 (s, 1H), 7.92-7.86 (m, 4H), 6.73 (d, J=7.6 Hz, 1H), 6.47 (d, J=7.8 Hz, 1H), 3.39-3.36 (m, 2H), 3.13-3.07 (m, 1H), 2.87-2.70 (m, 1H), 2.54 (s, 3H), 2.50-2.47 (m, 1H), 1.99 (s, 3H), 1.89-1.81 (m, 1H), 1.56-1.34 (m, 1H), 1.46-1.41 (m, 1H).

### Example 46: Preparation of 4-((4-aminopiperidin-1-yl)sulfonyl)-N-(3-cyano-4-methyl-1H-indol-7-yl) benzenesulfonamide; Compound 81

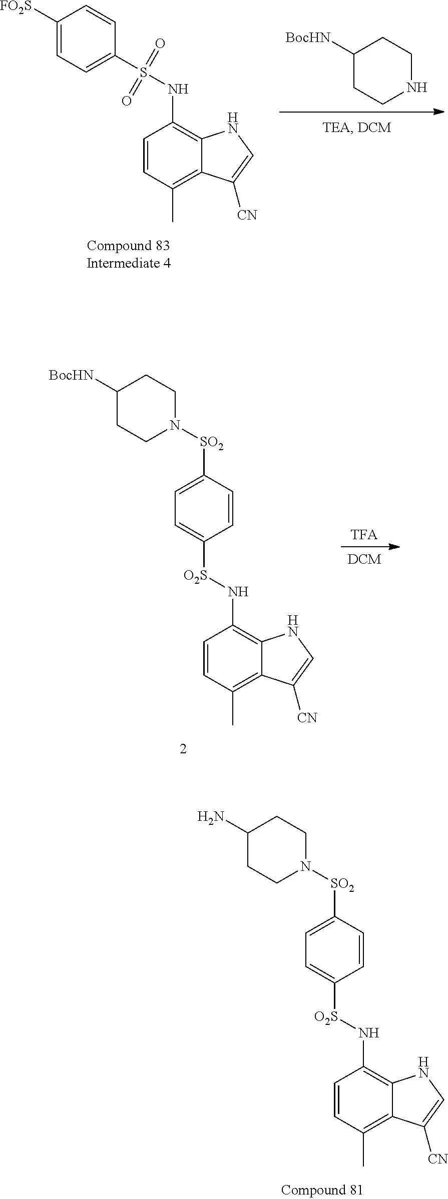

### 1. Preparation of tert-butyl (1-((4-(N-(3-cyano-4-methyl-1H-indol-7-yl)sulfamoyl)phenyl) sulfonyl)piperidin-4-yl)carbamate

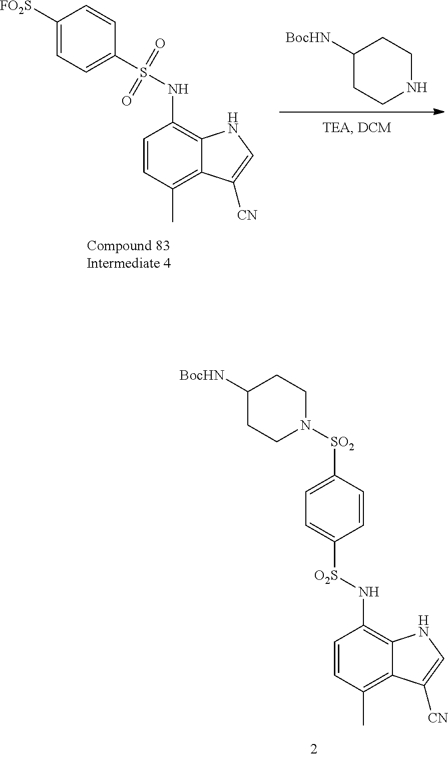

To a solution of 4-(N-(3-cyano-4-methyl-1H-indol-7-yl)sulfamoyl)benzenesulfonyl fluoride (50.0 mg, 0.12 mmol) and TEA (38.4 mg, 0.38 mmol) in DCM (2 mL) was added tert-butyl piperidin-4-ylcarbamate (28.0 mg, 0.14 mmol). The mixture was stirred for 18 h at room temperature. The mixture was concentrated to afford a crude product, which was purified by column chromatography on silica gel eluting with PE/EA to give tert-butyl (1-((4-(N-(3-cyano-4-methyl-1H-indol-7-yl)sulfamoyl)phenyl)sulfonyl)piperidin-4-yl)carbamate (43.6 mg, yield: 62.3%) as a white solid. LCMS (ESI) found: 574 [M+H]⁺.

### 2. Preparation of 4-((4-aminopiperidin-1-yl)sulfonyl)-N-(3-cyano-4-methyl-1H-indol-7-yl) benzenesulfonamide

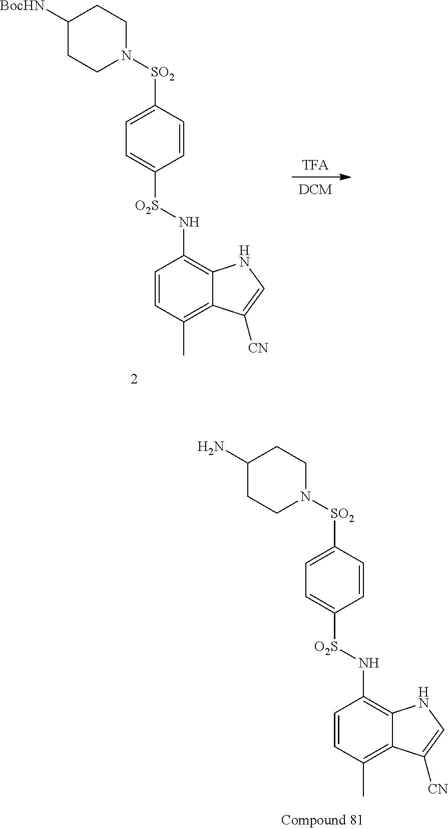

To a solution of tert-butyl (1-((4-(N-(3-cyano-4-methyl-1H-indol-7-yl)sulfamoyl)phenyl) sulfonyl)piperidin-4-yl) carbamate (43.6 mg, 0.07 mmol) in DCM (2 mL) was added TFA (1 mL). The mixture was stirred at rt for 2 h. The mixture was concentrated. The residue was basified with 10% aqueous NaHCO₃ (20 mL) and extracted with DCM (20 mL×3). The combined organic layer was purified by prep-HPLC to give 4-((4-aminopiperidin-1-yl)sulfonyl)-N-(3-cyano-4-methyl-1H-indol-7-yl) benzenesulfonamide (Compound 81, 17.7 mg, yield: 53.3%) as a white solid. LCMS (ESI) found: 474 [M+H]⁺. ¹H NMR (400 MHz, CD₃OD) δ 7.93 (s, 1H), 7.89-7.79 (m, 4H), 6.77 (d, J=8.2 Hz, 1H), 6.47 (d, J=7.7 Hz, 1H), 3.85 (d, J=12.3 Hz, 2H), 3.17-3.01 (m, 1H), 2.66 (s, 3H), 2.44-2.32 (m, 2H), 2.07-2.01 (m, 2H), 1.71-1.56 (m, 2H).

### Example 47: Preparation of 4-((3-aminopiperidin-1-yl)sulfonyl)-N-(3-cyano-4-methyl-1H-indol-7-yl) benzenesulfonamide; Compound 82

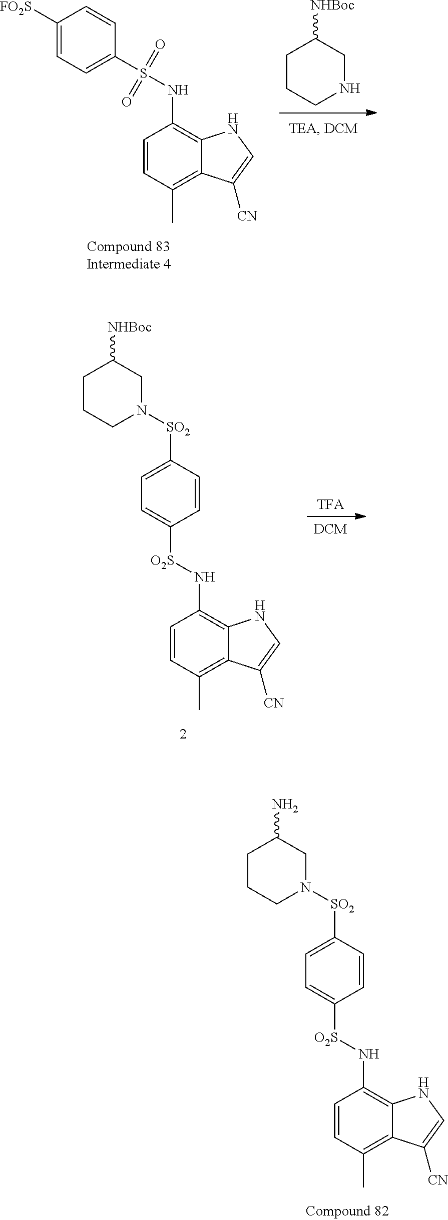

### 1. Preparation of tert-butyl (1-((4-(N-(3-cyano-4-methyl-1H-indol-7-yl)sulfamoyl)phenyl) sulfonyl)piperidin-3-yl)carbamate

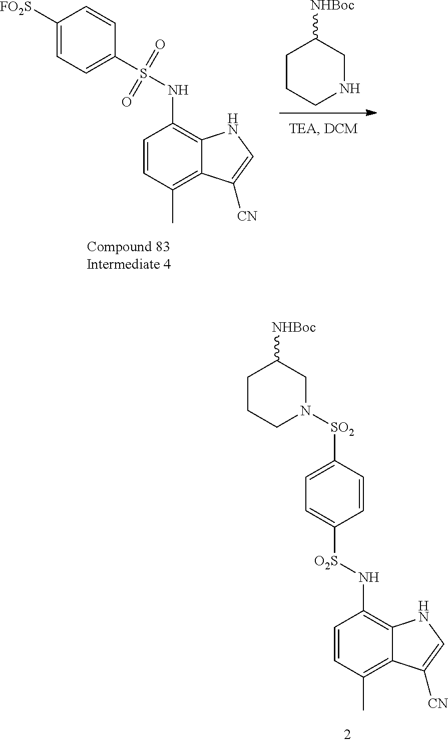

To a solution of 4-(N-(3-cyano-4-methyl-1H-indol-7-yl)sulfamoyl)benzenesulfonyl fluoride (50.0 mg, 0.12 mmol) and TEA (38.4 mg, 0.38 mmol) in DCM (2 mL), was added tert-butyl piperidin-3-ylcarbamate (28.0 mg, 0.14 mmol). The mixture was stirred for 18 h at room temperature. The mixture was concentrated to afford a crude product, which was purified by column chromatography on silica gel eluting with PE/EA to give tert-butyl (1-((4-(N-(3-cyano-4-methyl-1H-indol-7-yl)sulfamoyl)phenyl)sulfonyl)piperidin-3-yl)carbamate (47.6 mg, yield: 69.2%) as a white solid. LCMS (ESI) found: 574 [M+H]⁺.

### 2. Preparation of 4-((3-aminopiperidin-1-yl)sulfonyl)-N-(3-cyano-4-methyl-1H-indol-7-yl) benzenesulfonamide

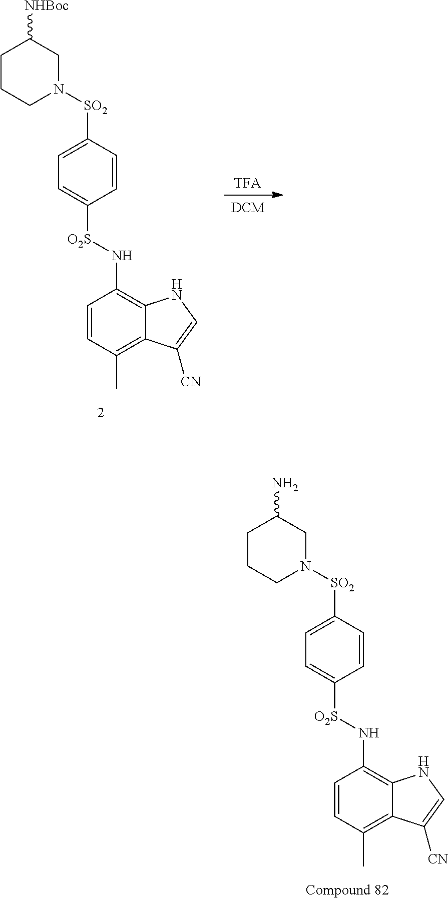

To a solution of tert-butyl (1-((4-(N-(3-cyano-4-methyl-1H-indol-7-yl)sulfamoyl)phenyl) sulfonyl)piperidin-3-yl)carbamate (47.6 mg, 0.08 mmol) in DCM (2 mL) was added TFA (1 mL). The mixture was stirred at rt for 2 h. The mixture was concentrated. The residue was basified with 10% aqueous NaHCO₃ (20 mL) and extracted with DCM (20 mL×3). The combined organic layer was purified by prep-HPLC to give 4-((3-aminopiperidin-1-yl)sulfonyl)-N-(3-cyano-4-methyl-1H-indol-7-yl)benzenesulfonamide (Compound 82, 33.7 mg, yield: 87.5%) as a white solid. LCMS (ESI) found: 474 [M+H]⁺. ¹H NMR (400 MHz, CD₃OD) δ 7.94 (s, 1H), 7.88-7.85 (m, 4H), 6.78 (d, J=7.8 Hz, 1H), 6.46 (d, J=7.7 Hz, 1H), 3.39-3.34 (m, 2H), 3.02-2.94 (m, 3H), 2.66 (s, 3H), 1.90-1.88 (m, 2H), 1.70-1.65 (m, 1H), 1.59-1.52 (m, 1H).

### Example 48: Preparation of N-(3-cyano-4-methyl-1H-indol-7-yl)-4-(piperazin-1-ylsulfonyl)benzene sulfonamide; Compound 83

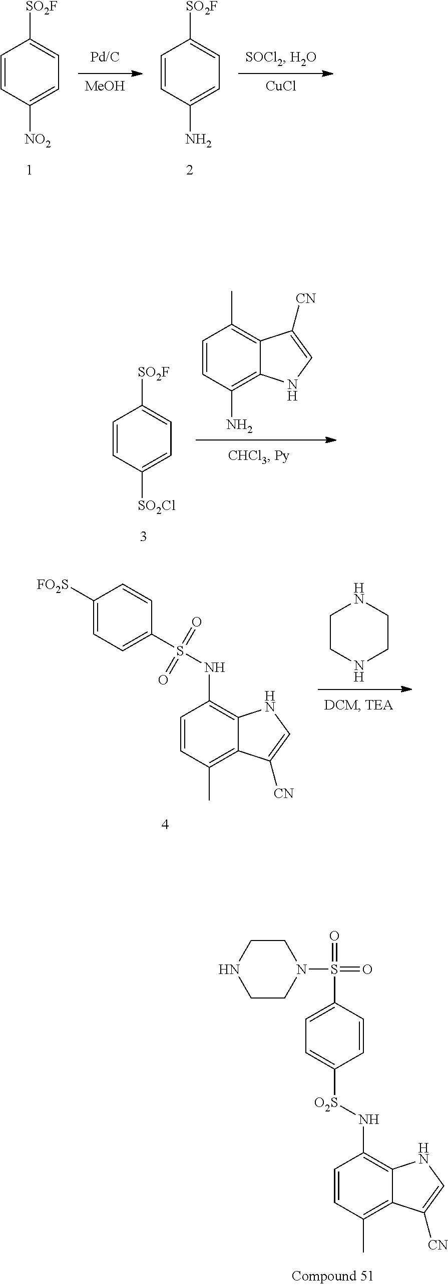

### 1. Preparation of 4-aminobenzenesulfonyl fluoride

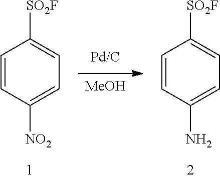 [SMILES: "MeOH" ; O=[N+]([O-])c1ccc(S(=O)(=O)F)cc1 ; Nc1ccc(S(=O)(=O)F)cc1]

To a solution of 4-nitrobenzenesulfonyl fluoride (5.0 g, 24.39 mol) in MeOH (50 mL) was added Pd/C (0.5 g). The mixture was charged with H₂ for three times. The suspension was heated to 55° C. and stirred for 18 h under H₂ atmosphere. TLC (PE/EA=3:1, R_{f}=0.5) showed the starting material was consumed. The mixture was filtered through a pad of celite. The filtrate was concentrated under reduced pressure to give a crude product, which was purified by column chromatography on silica gel eluting with PE/DCM from 5/1 to 1/1 to give 4-aminobenzenesulfonyl fluoride (2.2 g, yield: 51.5%) as a white solid. LCMS (ESI) found: 176 [M+H]⁺. ¹H NMR (400 MHz, DMSO-d6) δ 7.76-7.47 (m, 2H), 6.83-6.54 (m, 4H).

### 2. Preparation of 4-(chlorosulfonyl)benzenesulfonyl fluoride

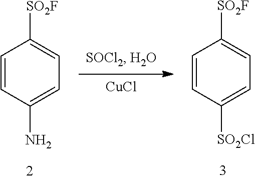

A solution of SOCl₂ (1.7 mL, 22.85 mmol) in ice-water (7 mL) was stirred at rt for 48 h. CuCl (22.6 mg, 0.23 mmol) was added at −5° C. and stirred for 10 min to give a solution A. To a solution of 4-aminobenzenesulfonyl fluoride (1.0 g, 5.71 mmol) in HCl (10 mol/L, 5.7 mL) was added NaNO₂ (0.4 g, 6.29 mmol) in H₂O (2 mL) at −10° C. and stirred for 20 min to give a solution B. The solution B was added dropwise to solution A at −5° C. and stirred for 3 h. The mixture was extracted with DCM (50 mL×3). The combined organic layer was washed with brine (50 mL), dried over anhydrous Na₂SO₄, and then concentrated to give a crude product, which was triturated with hexane to give 4-(chlorosulfonyl)benzenesulfonyl fluoride (1.0 g, yield: 67.7%) as a yellow solid. ¹H NMR (400 MHz, CDCl₃): δ 8.46-8.25 (m, 4H).

### 3. Preparation of 4-(N-(3-cyano-4-methyl-1H-indol-7-yl)sulfamoyl) benzenesulfonyl fluoride

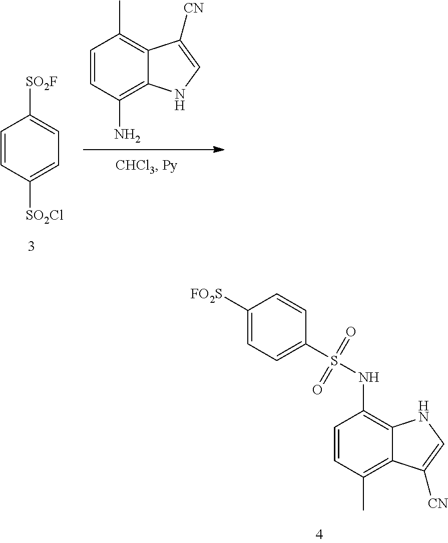

To a solution of 7-amino-4-methyl-1H-indole-3-carbonitrile (661.8 mg, 3.87 mmol) and pyridine (917.2 mg, 11.61 mmol) in CHCl₃ (2 mL) was added 4-(chlorosulfonyl) benzenesulfonyl fluoride (1.0 g, 3.87 mmol). The mixture was stirred at rt for 2 h. LCMS showed the reaction was completed. The mixture was concentrated under reduced pressure to give a crude product, which was purified by prep-TLC to give 4-(N-(3-cyano-4-methyl-1H-indol-7-yl)sulfamoyl)benzenesulfonyl fluoride (1.2 g, yield: 79.1%) as a solid. LCMS (ESI) found: 394 [M+H]⁺.

### 4. Preparation of N-(3-cyano-4-methyl-1H-indol-7-yl)-4-(piperazin-1-ylsulfonyl)benzenesulfonamide

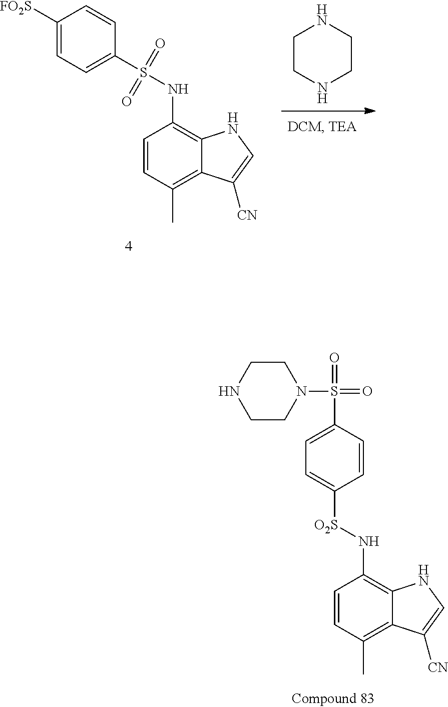

To a solution of 4-(N-(3-cyano-4-methyl-1H-indol-7-yl)sulfamoyl)benzenesulfonyl fluoride (14.3 mg, 0.04 mmol) and TEA (7.1 mg, 0.07 mmol) in DCM (2 mL) was added piperazine (11.2 mg, 0.13 mmol). The mixture was stirred at rt for 18 h. LCMS showed the reaction was completed. The mixture was concentrated under reduced pressure. The residue was purified by prep-TLC to give a crude product, which was further purified by prep-HPLC to give N-(3-cyano-4-methyl-1H-indol-7-yl)-4-(piperazin-1-yl sulfonyl)benzene sulfonamide (Compound 83, 10.3 mg, yield: 61.5%) as a white solid. LCMS (ESI) found: 460 [M+H]⁺. ¹H NMR (400 MHz, DMSO-d6) δ 11.95 (s, 1H), 8.10 (s, 1H), 7.91 (d, J=8.5 Hz, 2H), 7.83 (d, J=8.5 Hz, 2H), 6.75 (d, J=7.8 Hz, 1H), 6.54 (d, J=7.7 Hz, 1H), 2.83 (s, 8H), 2.55 (s, 3H).

### Example 49: Preparation of N-(3-cyano-4-methyl-1H-indol-7-yl)-4-((3-ethylpiperazin-1-yl)sulfonyl) benzenesulfonamide; Compound 84

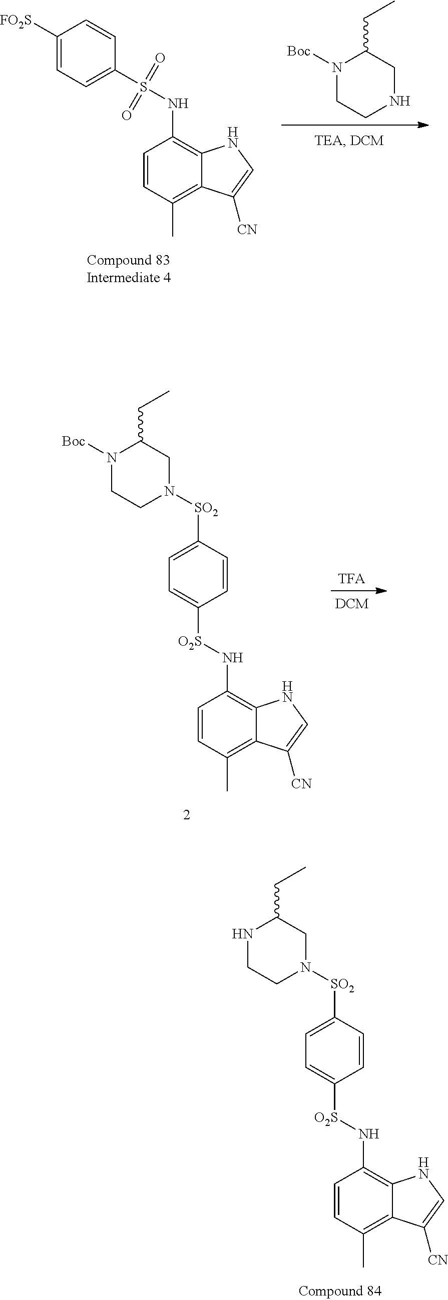

### 1. Preparation of tert-butyl 4-((4-(N-(3-cyano-4-methyl-1H-indol-7-yl)sulfamoyl)phenyl) sulfonyl)-2-ethylpiperazine-1-carboxylate

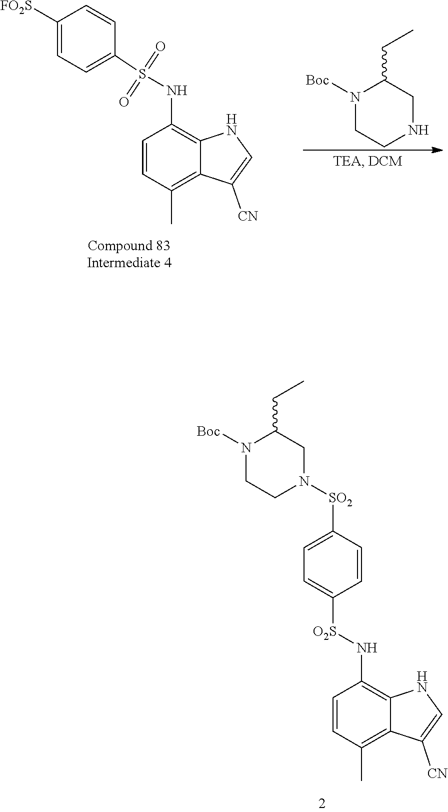

To a solution of 4-(N-(3-cyano-4-methyl-1H-indol-7-yl)sulfamoyl)benzenesulfonyl fluoride (25.0 mg, 0.06 mmol) and TEA (19.2 mg, 0.19 mmol) in DCM (2 mL) was added tert-butyl 2-ethylpiperazine-1-carboxylate (15.0 mg, 0.07 mmol). The mixture was stirred for 18 h at room temperature. The mixture was concentrated to afford a crude product, which was purified by column chromatography on silica gel eluting with PE/EA to give tert-butyl 4-((4-(N-(3-cyano-4-methyl-1H-indol-7-yl)sulfamoyl)phenyl)sulfonyl)-2-methylpiperazine-1-carboxylate (19.2 mg, yield: 55.8%) as a white solid. LCMS (ESI) found: 588 [M+H]⁺.

### 2. Preparation of N-(3-cyano-4-methyl-1H-indol-7-yl)-4-((3-ethylpiperazin-1-yl)sulfonyl) benzenesulfonamide

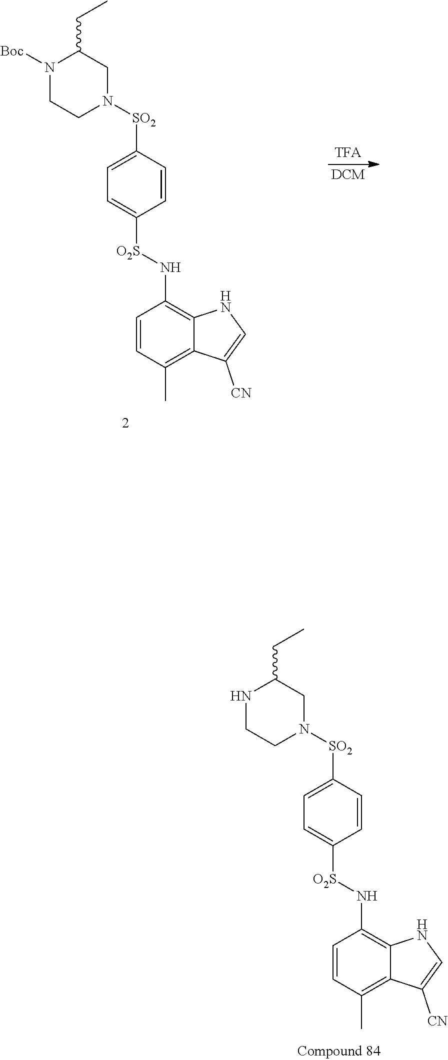 [SMILES: CCC1CN(S(=O)(=O)c2ccc(S(=O)(=O)Nc3ccc(C)c4c(C#N)c[nH]c34)cc2)CCN1C(=O)OC(C)(C)C ; CCC1CN(S(=O)(=O)c2ccc(S(=O)(=O)Nc3ccc(C)c4c(C#N)c[nH]c34)cc2)CCN1]

To a solution of tert-butyl 4-((4-(N-(3-cyano-4-methyl-1H-indol-7-yl)sulfamoyl)phenyl) sulfonyl)-2-methylpiperazine-1-carboxylate (19.2 mg, 0.03 mmol) in DCM (2 mL) was added TFA (1 mL). The mixture was stirred at rt for 2 h. The mixture was concentrated. The residue was basified with 10% aqueous NaHCO₃ (20 mL) and extracted with DCM (20 mL×3). The combined organic layer was concentrated to give a crude product, which was purified by prep-HPLC to give N-(3-cyano-4-methyl-1H-indol-7-yl)-4-((3-methylpiperazin-1-yl)sulfonyl) benzenesulfonamide (Compound 84, 5.1 mg, yield: 35.9%) as a white solid. LCMS (ESI) found: 488 [M+H]⁺. ¹H NMR (400 MHz, DMSO-d6) δ 11.98 (s, 1H) 8.13 (s, 1H), 7.92-7.86 (m, 4H), 6.77 (d, J=7.8 Hz, 1H), 6.47 (d, J=7.7 Hz, 1H), 3.51-3.43 (m, 2H), 3.06-2.94 (m, 1H), 2.72-2.66 (m, 2H), 2.55 (s, 3H), 2.27-2.19 (m, 1H), 1.93-1.87 (m, 1H), 1.37-1.30 (m, 2H), 1.22 (s, 1H), 0.86 (t, J=7.5 Hz, 3H).

### Example 50: Preparation of N-(3-cyano-4-methyl-1H-indol-7-yl)-4-((4-(2-methoxyethyl)piperazin-1-yl) sulfonyl)benzenesulfonamide; Compound 85

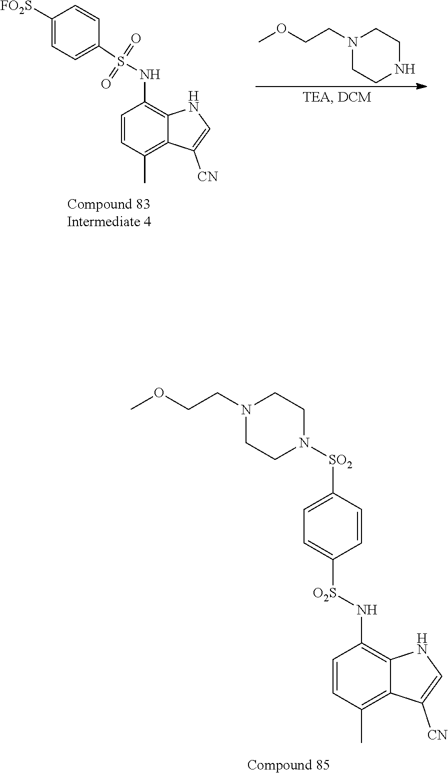

### 1. Preparation of N-(3-cyano-1H-indol-7-yl)-4-((4-(2-methoxyethyl)piperazin-1-yl)sulfonyl) benzenesulfonamide

To a solution of 4-(N-(3-cyano-4-methyl-1H-indol-7-yl)sulfamoyl)benzenesulfonyl fluoride (25.0 mg, 0.06 mmol) and TEA (19.2 mg, 0.19 mmol) in DCM (2 mL) was added 1-(2-methoxyethyl)piperazine (9.1 mg, 0.06 mmol. The mixture was stirred for 18 h at room temperature. The mixture was concentrated to give a crude product, which was purified to was purified by column chromatography on silica gel eluting with PE/EA to give N-(3-cyano-1H-indol-7-yl)-4-((4-(2-methoxyethyl)piperazin-1-yl)sulfonyl)benzenesulfonamide (Compound 85, 17.3 mg, yield: 53.0%) as a white solid. LCMS (ESI) found: 518 [M+H]⁺. ¹H NMR (400 MHz, DMSO-d6) δ 11.89 (s, 1H), 10.22 (s, 1H), 8.14 (s, 1H), 7.84-7.81 (m, 4H), 6.78 (d, J=7.8 Hz, 1H), 6.50 (d, J=7.7 Hz, 1H), 3.54-3.35 (m, 2H), 3.19 (s, 3H), 2.83 (s, 4H), 2.56 (s, 3H), 2.48-2.45 (m, 6H).

### Example 51: Preparation of N-(3-cyano-4-methyl-1H-indol-7-yl)-4-((4-(oxetan-3-yl)piperazin-1-yl) sulfonyl)benzenesulfonamide; Compound 86

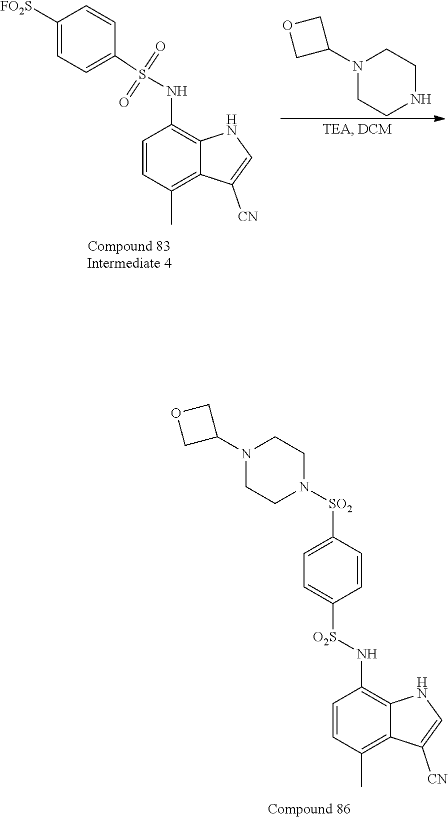

### 1. Preparation of N-(3-cyano-4-methyl-1H-indol-7-yl)-4-((4-(oxetan-3-yl)piperazin-1-yl) sulfonyl)benzenesulfonamide

To a solution of 4-(N-(3-cyano-4-methyl-1H-indol-7-yl)sulfamoyl)benzenesulfonyl fluoride (50.0 mg, 0.12 mmol) and TEA (38.4 mg, 0.38 mmol) in DCM (2 mL) was added 1-(oxetan-3-yl)piperazine (28.4 mg, 0.20 mmol). The mixture was stirred for 18 h at room temperature. The mixture was concentrated to afford a crude product, which was purified by column chromatography on silica gel eluting with PE/EA to give N-(3-cyano-4-methyl-1H-indol-7-yl)-4-((4-(oxetan-3-yl)piperazin-1-yl) sulfonyl) benzene sulfonamide (Compound 86, 37.9 mg, yield: 58.3%) as a white solid. LCMS (ESI) found: 516 [M+H]⁺. ¹H NMR (400 MHz, DMSO-d6) δ 11.95 (s, 1H), 10.09 (s, 1H), 8.14 (s, 1H), 7.90-7.85 (m, 4H), 6.80 (d, J=7.7 Hz, 1H), 6.51 (d, J=7.7 Hz, 1H), 4.51 (t, J=6.5 Hz, 2H), 4.38 (t, J=6.5 Hz, 2H), 3.57 (s, 1H), 2.90 (s, 4H), 2.58 (s, 3H), 2.31 (s, 4H).

### Example 52: Preparation of N-(4-chloro-1H-indol-7-yl)-4-(piperazin-1-ylsulfonyl)benzenesulfonamide; Compound 87

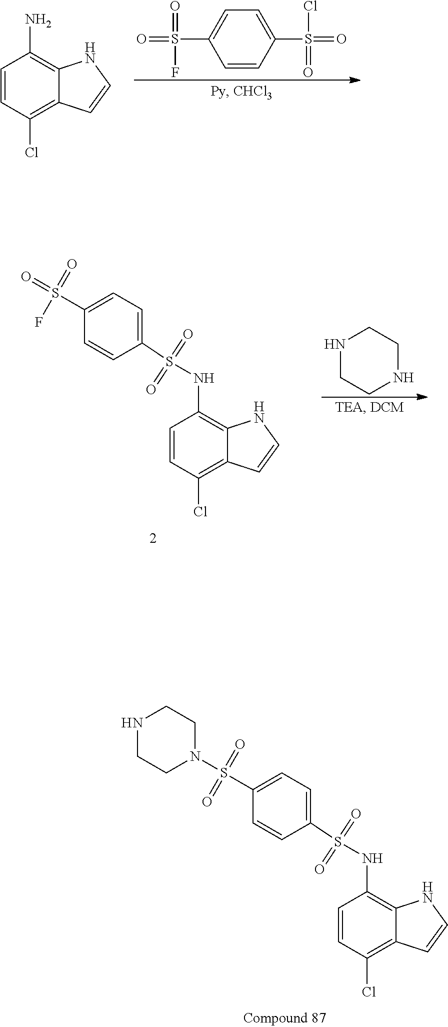 [SMILES: Nc1ccc(Cl)c2cc[nH]c12 ; O=S(=O)(F)c1ccc(S(=O)(=O)Cl)cc1 ; O=S(=O)(F)c1ccc(S(=O)(=O)Nc2ccc(Cl)c3cc[nH]c23)cc1 ; C1CNCCN1 ; O=S(=O)(Nc1ccc(Cl)c2cc[nH]c12)c1ccc(S(=O)(=O)N2CCNCC2)cc1]

### 1. Preparation of 4-(N-(4-chloro-1H-indol-7-yl)sulfamoyl)benzene-1-sulfonyl fluoride

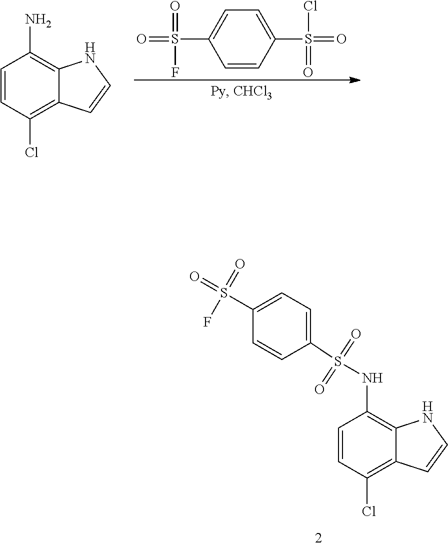 [SMILES: Nc1ccc(Cl)c2cc[nH]c12 ; O=S(=O)(F)c1ccc(S(=O)(=O)Cl)cc1 ; O=S(=O)(F)c1ccc(S(=O)(=O)Nc2ccc(Cl)c3cc[nH]c23)cc1]

To a solution of 4-chloro-1H-indol-7-amine (30.0 mg, 0.22 mmol) and pyridine (52.1 mg, 0.66 mmol) in CHCl₃ (3.0 mL) was added 4-(chlorosulfonyl)benzene-1-sulfonyl fluoride (38.6 mg, 0.22 mmol). The mixture was stirred for 1 h at room temperature. The mixture was diluted with water (50 mL) and extracted with DCM (50 mL×3). The combined organic layer was washed with water (20 mL), brine (20 mL), dried over Na₂SO₄, filtered and concentrated under reduced pressure to afford the crude product, which was purified by column chromatography on silica gel eluted with PE/EA from 10/1 to 2/1 to give 4-(N-(4-chloro-1H-indol-7-yl) sulfamoyl)benzene-1-sulfonyl fluoride (53.0 mg, 78.9%) as a white solid. LCMS (ESI) found: 389 [M+H]⁺.

### 2. Preparation of N-(4-chloro-1H-indol-7-yl)-4-(piperazine-1-sulfonyl)benzene-1-sulfon amide

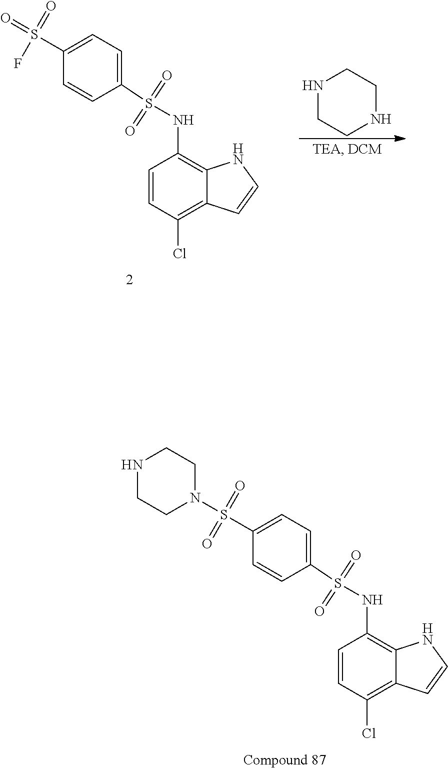 [SMILES: O=S(=O)(F)c1ccc(S(=O)(=O)Nc2ccc(Cl)c3cc[nH]c23)cc1 ; C1CNCCN1 ; O=S(=O)(Nc1ccc(Cl)c2cc[nH]c12)c1ccc(S(=O)(=O)N2CCNCC2)cc1]

To a solution of 4-(N-(4-chloro-1H-indol-7-yl)sulfamoyl)benzene-1-sulfonyl fluoride (53.0 mg, 0.17 mmol) in DCM (4.0 mL) was added TEA (51.5 mg, 0.51 mmol) and piperazine (43.0 mg, 0.50 mmol). The mixture was stirred at rt for 18 h. The reaction mixture was diluted with water (50 mL) and extracted with DCM (50 mL×3). The combined organic layer was washed with water (20 mL), brine (20 mL), dried over Na₂SO₄, filtered and then concentrated under reduced pressure to afford the crude product, which was purified by column chromatography on silica gel eluted with DCM/MeOH from pure DCM to DCM/MeOH=10/1 to give N-(4-chloro-1H-indol-7-yl)-4-(piperazine-1-sulfonyl)benzene-1-sulfonamide (Compound 87, 20.0 mg, 25.4%) as a white solid. LCMS (ESI) found: 455 [M+H]⁺. ¹H NMR (400 MHz, DMSO-d6) δ 11.09 (s, 1H), 7.93 (d, J=8.4 Hz, 2H), 7.80 (d, J=8.4 Hz, 2H), 7.31 (t, J=2.7 Hz, 1H), 6.83 (d, J=8.1 Hz, 1H), 6.60 (d, J=8.1 Hz, 1H), 6.38-6.31 (m, 1H), 2.79-2.77 (m, 4H), 2.75-2.73 (m, 4H).

### Example 53: Synthesis of N-(3-chloro-4-cyano-1H-indol-7-yl)-4-(piperazin-1-ylsulfonyl)benzene sulfonamide; Compound 88

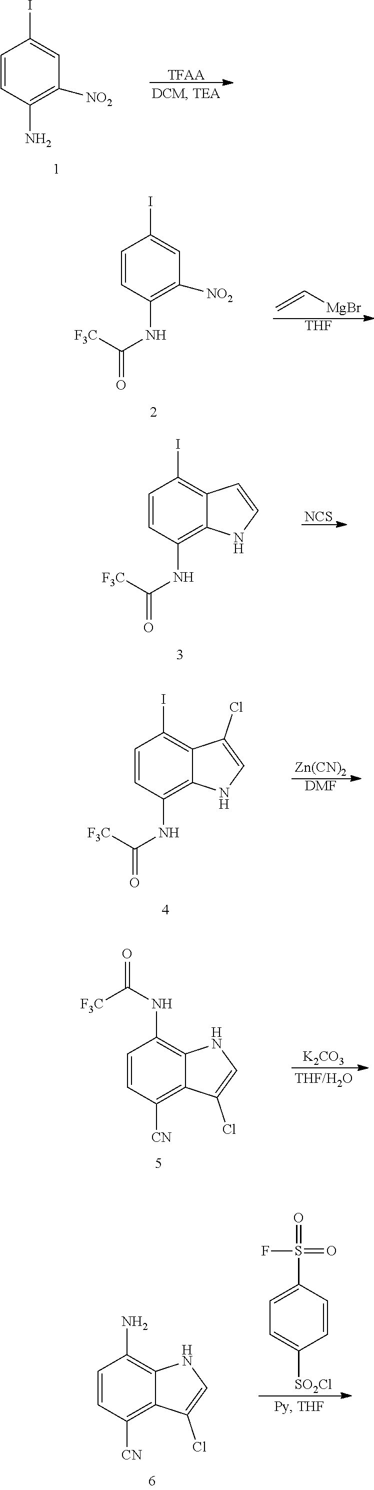

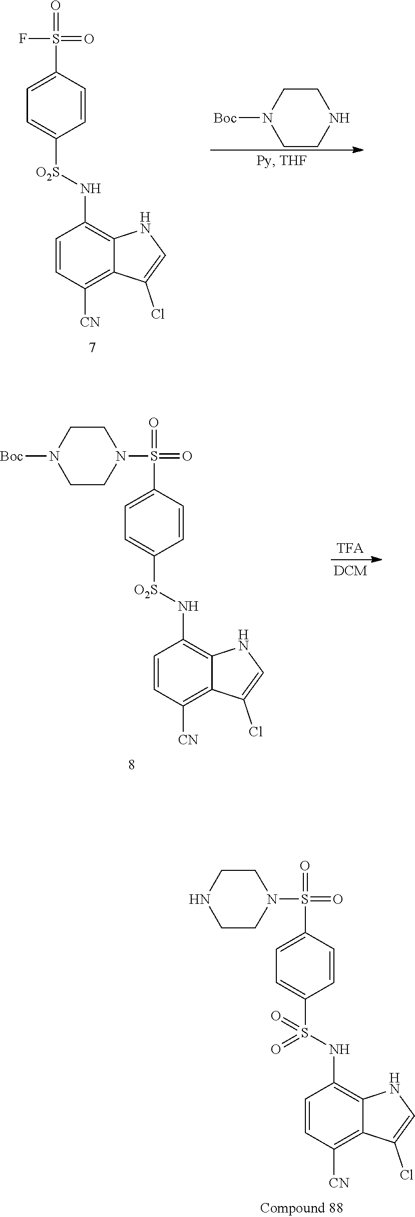 [SMILES: N#Cc1ccc(NS(=O)(=O)c2ccc(S(=O)(=O)F)cc2)c2[nH]cc(Cl)c12 ; CC(C)(C)OC(=O)N1CCNCC1 ; CC(C)(C)OC(=O)N1CCN(S(=O)(=O)c2ccc(S(=O)(=O)Nc3ccc(C#N)c4c(Cl)c[nH]c34)cc2)CC1 ; N#Cc1ccc(NS(=O)(=O)c2ccc(S(=O)(=O)N3CCNCC3)cc2)c2[nH]cc(Cl)c12]

### 1. Preparation of N-(3-chloro-4-cyano-1H-indol-7-yl)-2,2,2-trifluoroacetamide

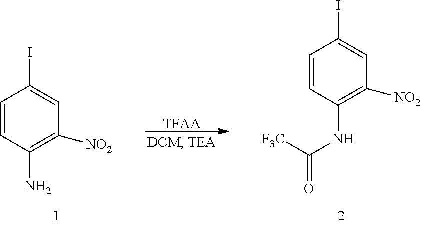 [SMILES: Nc1ccc(I)cc1[N+](=O)[O-] ; "TFAA" ; O=C(Nc1ccc(I)cc1[N+](=O)[O-])C(F)(F)F]

To a solution of 4-iodo-2-nitroaniline (25.0 g, 94.69 mmol) in DCM (250 mL) were added TEA (19.1 g, 189.34 mmol) and TFAA (21.9 g, 104.16 mmol) at 0° C. The mixture was stirred at rt for 2 h. After completion, the mixture was diluted with water (150 mL) and extracted with DCM (150 mL×3). The combined organic layer was dried over MgSO₄, filtered and concentrated to give a crude product, which was purified by column chromatography on silica gel eluting with DCM/MeOH from 35/1 to 25/1 to give 2,2,2-trifluoro-N-(4-iodo-2-nitrophenyl)acetamide (30.0 g, yield: 88.0%) as a yellow solid. LCMS (ESI) found: 361 [M+H]⁺.

### 2. Preparation of N-(3-chloro-4-cyano-1H-indol-7-yl)-2,2,2-trifluoroacetamide

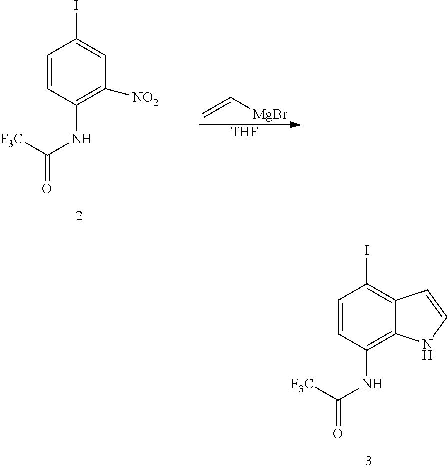

To a solution of 2,2,2-trifluoro-N-(4-iodo-2-nitrophenyl)acetamide (15.0 g, 41.55 mmol) in THF (100 mL) under N₂ atmosphere was added slowly bromo(vinyl)magnesium (207.75 mL, 207.75 mmol) at −78° C. The mixture was gradually warmed to rt and stirred at rt for 2 h. The mixture was quenched with saturated NH₄Cl (250 mL) and extracted with EA (250 mL×3). The combined organic layer was concentrated to give a crude product, which was purified by column chromatography on silica gel to eluting with PE/EA from 10/1 to 3/1 to give 2,2,2-trifluoro-N-(4-iodo-1H-indol-7-yl)acetamide (2.5 g, yield: 16.9%) as a light yellow solid. LCMS (ESI): 355 [M+H]⁺.

### 3. Preparation of N-(3-chloro-4-cyano-1H-indol-7-yl)-2,2,2-trifluoroacetamide

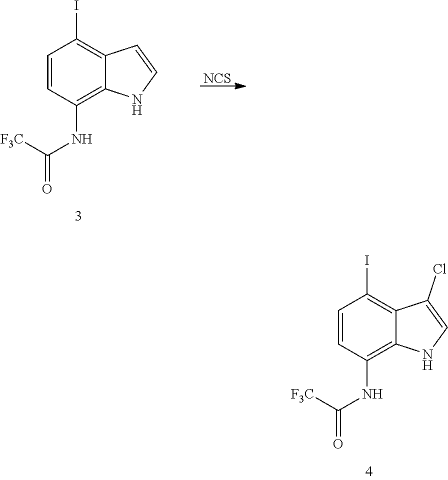 [SMILES: O=C(Nc1ccc(I)c2cc[nH]c12)C(F)(F)F ; "NCS" ; O=C(Nc1ccc(I)c2c(Cl)c[nH]c12)C(F)(F)F]

To a solution of 2,2,2-trifluoro-N-(4-iodo-1H-indol-7-yl)acetamide (4.5 g, 12.68 mmol) in THF/DMF (100/1, 50 mL) was added NCS (1.7 g, 12.68 mmol). The mixture was stirred at rt for 5 h. LCMS showed the desired mass was detected. The mixture was quenched with sat. Na₂S₂O₃ (50 mL) and extracted with DCM (50 mL×3). The combined organic layer was concentrated to give a crude product, which was purified by flash column eluting with PE/EA from 100/0 to 85/15 to give N-(3-chloro-4-iodo-1H-indol-7-yl)-2,2,2-trifluoro acetamide (2.3 g, yield: 46.6%) as a white solid. LCMS (ESI): 389 [M+H]⁺.

### 4. Preparation of N-(3-chloro-4-cyano-1H-indol-7-yl)-2,2,2-trifluoroacetamide

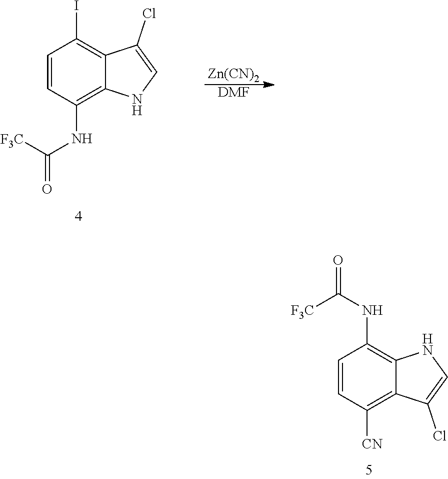

To a solution of N-(3-chloro-4-iodo-1H-indol-7-yl)-2,2,2-trifluoroacetamide (500.0 mg, 1.29 mmol) in DMF (10 mL) were added Zn(CN)₂ (755.6 mg, 6.43 mmol) and Pd(PPh₃)₄ (150.3 mg, 0.13 mmol). The mixture was stirred at 110° C. for 2 h under N₂ atmosphere. The mixture was quenched with sat. NaHCO₃ (20 mL) and extracted with EA (25 mL×3). The combined organic layer was dried over MgSO₄, filtered and concentrated to give a crude product, which was purified by flash eluting with PE/EA from 15/1 to 10/1 to give N-(3-chloro-4-cyano-1H-indol-7-yl)-2,2,2-trifluoroacetamide (213.0 mg, yield: 57.4%) as a white solid. LCMS (ESI) found: 288 [M+H]⁺.

### 5. Preparation of 7-amino-3-chloro-1H-indole-4-carbonitrile

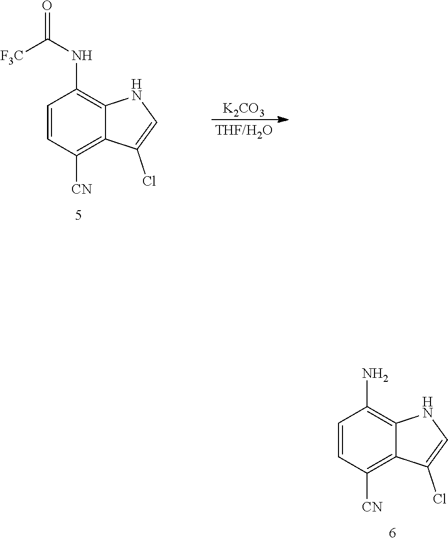

To a solution of N-(3-chloro-4-cyano-1H-indol-7-yl)-2,2,2-trifluoroacetamide (213.0 mg, 0.74 mmol) in THF/H₂O (4/1, 5 mL) was added K₂CO₃ (409.1 mg, 2.96 mmol). The mixture was stirred at rt for 18 h until the starting material was consumed completely. The reaction mixture was filtered and the filtrate was concentrated to give a crude product, which was purified by flash column to give 7-amino-3-chloro-1H-indole-4-carbonitrile (136.2 mg, yield: 96.1%) as a white solid. LCMS (ESI) found: 192.6 [M+H]⁺.

### 6. Preparation of 4-(N-(3-chloro-4-cyano-1H-indol-7-yl)sulfamoyl)benzenesulfonyl fluoride

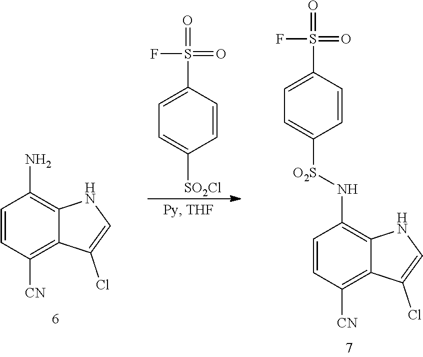 [SMILES: N#Cc1ccc(N)c2[nH]cc(Cl)c12 ; O=S(=O)(F)c1ccc(S(=O)(=O)Cl)cc1 ; N#Cc1ccc(NS(=O)(=O)c2ccc(S(=O)(=O)F)cc2)c2[nH]cc(Cl)c12]

To a solution of 7-amino-3-chloro-1H-indole-4-carbonitrile (136.2 mg, 0.71 mmol) and Pyridine (0.5 mL) in THF (10 mL) was added 4-(chlorosulfonyl)benzene-1-sulfonyl fluoride (367.3 mg, 1.42 mmol). The mixture was stirred at rt for 2 h until the starting material was consumed completely. The reaction mixture was concentrated to give a crude product, which was purified by flash column eluting with PE/EA from 3/1 to 1/1 to give 4-(N-(3-chloro-4-cyano-1H-indol-7-yl)sulfamoyl) benzenesulfonyl fluoride (110.0 mg, yield: 37.4%) as a white solid. LCMS (ESI) found: 414 [M+H]⁺.

### 7. Preparation of tert-butyl 4-((4-(N-(3-chloro-4-cyano-1H-indol-7-yl)sulfamoyl)phenyl)sulfonyl)piperazine-1-carboxylate

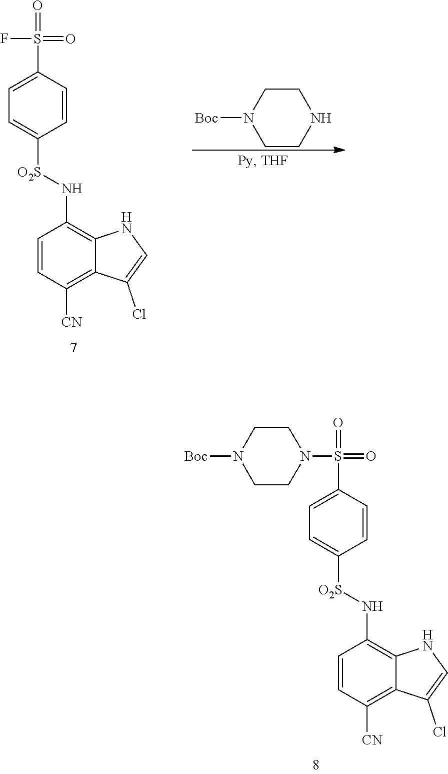 [SMILES: N#Cc1ccc(NS(=O)(=O)c2ccc(S(=O)(=O)F)cc2)c2[nH]cc(Cl)c12 ; CC(C)(C)OC(=O)N1CCNCC1 ; CC(C)(C)OC(=O)N1CCN(S(=O)(=O)c2ccc(S(=O)(=O)Nc3ccc(C#N)c4c(Cl)c[nH]c34)cc2)CC1]

To a solution of 4-(N-(3-chloro-4-cyano-1H-indol-7-yl)sulfamoyl)benzenesulfonyl fluoride (110.0 mg, 0.27 mmol) and Pyridine (128.0 mg, 1.62 mmol) in THF (10 mL) was added tert-butyl piperazine-1-carboxylate (100.6 mg, 0.54 mmol). The mixture was stirred at rt for 2 h until the starting material was consumed completely. The reaction mixture was concentrated to give a crude product, which was purified by flash to give tert-butyl 4-((4-(N-(3-chloro-4-cyano-1H-indol-7-yl) sulfamoyl)phenyl)sulfonyl)piperazine-1-carboxylate (100.0 mg, yield: 63.8%) as a white solid. LCMS (ESI) found: 582 [M+H]⁺.

### 8. Preparation of N-(3-chloro-4-cyano-1H-indol-7-yl)-4-(piperazin-1-ylsulfonyl)benzenesulfonamide

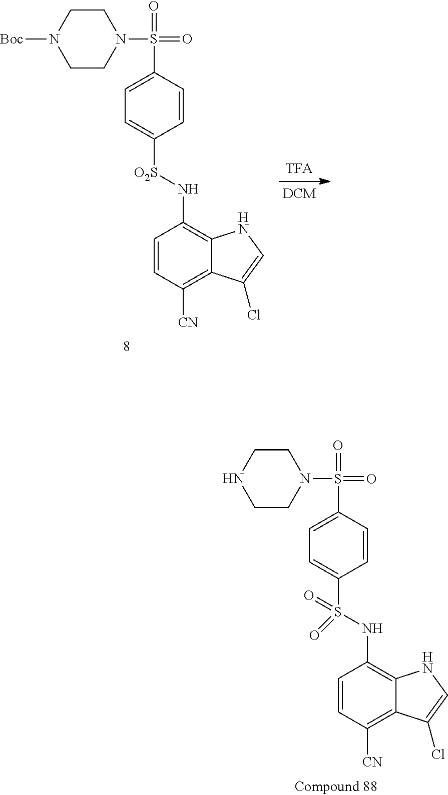 [SMILES: CC(C)(C)OC(=O)N1CCN(S(=O)(=O)c2ccc(S(=O)(=O)Nc3ccc(C#N)c4c(Cl)c[nH]c34)cc2)CC1 ; N#Cc1ccc(NS(=O)(=O)c2ccc(S(=O)(=O)N3CCNCC3)cc2)c2[nH]cc(Cl)c12]

To a solution of tert-butyl 4-((4-(N-(3-chloro-4-cyano-1H-indol-7-yl)sulfamoyl)phenyl)sulfonyl) piperazine-1-carboxylate (100.0 mg, 0.17 mmol) in DCM (3 mL) was added TFA (1.0 mL) slowly. The mixture was stirred at rt for 2 h until the starting material was consumed completely. The reaction mixture was concentrated and basified with 10% aqueous NaHCO₃ (15 mL) and extracted with DCM (25 mL×3). The combined organic layer was dried over MgSO₄, filtered and concentrated to give a crude product, which was purified by prep-HPLC to give N-(3-chloro-4-cyano-1H-indol-7-yl)-4-(piperazin-1-ylsulfonyl)benzenesulfonamide (HCOOH salt) (Compound 88, 37.7 mg, yield: 46.2%) as a white solid. LCMS (ESI) found: 480 [M+H]⁺. ¹H NMR (400 MHz, DMSO-d6) δ 11.54 (s, 1H), 8.14 (s, 0.74H), 8.04 (d, J=8.4 Hz, 2H), 7.81 (d, J=8.4 Hz, 2H), 7.32 (s, 1H), 7.18 (d, J=8.2 Hz, 1H), 6.91 (d, J=8.2 Hz, 1H), 3.18-3.13 (m, 4H), 3.07-3.01 (m, 4H).

### Example 54: Preparation of N-(3-cyano-4-methyl-1H-indol-7-yl)-4-((3-oxopiperazin-1-yl)sulfonyl) benzenesulfonamide; Compound 89

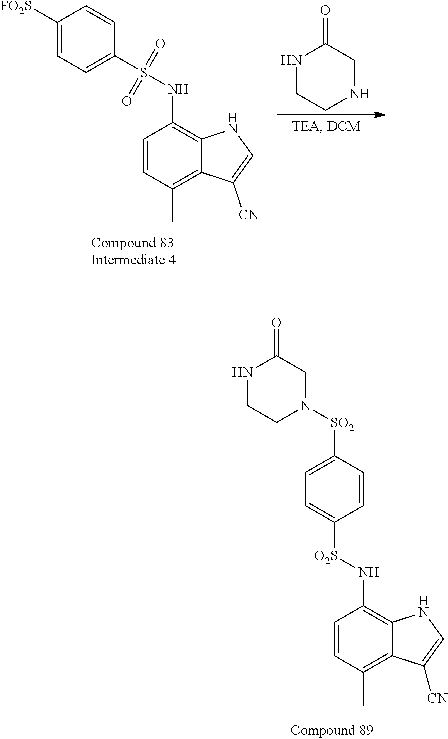

### 1. Preparation of N-(3-cyano-4-methyl-1H-indol-7-yl)-4-((3-oxopiperazin-1-yl)sulfonyl) benzenesulfonamide

To a solution of 4-(N-(3-cyano-4-methyl-1H-indol-7-yl)sulfamoyl)benzenesulfonyl fluoride (25.0 mg, 0.06 mmol) and TEA (19.2 mg, 0.19 mmol) in DCM (2 mL) was added piperazin-2-one (7.2 mg, 0.07 mmol). The mixture was stirred for 18 h at room temperature. The mixture was concentrated to afford a crude product, which was purified by column chromatography on silica gel eluting with PE/EA to give N-(3-cyano-4-methyl-1H-indol-7-yl)-4-((3-oxopiperazin-1-yl)sulfonyl)benzenesulfonamide (Compound 89, 8.6 mg, yield: 30.2%) as a white solid. LCMS (ESI) found: 474 [M+H]⁺. ¹H NMR (400 MHz, DMSO-d6) δ 8.06 (s, 1H), 8.00 (s, 1H), 7.89-7.87 (m, 4H), 6.86-6.66 (m, 1H), 6.55-6.53 (m, 1H), 3.52 (s, 2H), 3.18-3.17 (m, 4H), 2.56 (s, 3H).

### Example 55: Preparation of N-(3-cyano-4-methyl-1H-indol-7-yl)-4-(morpholinosulfonyl)benzene sulfonamide; Compound 90

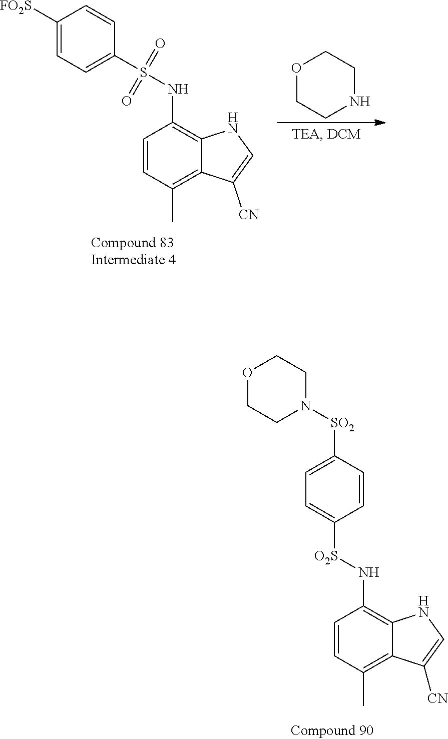

### 1. Preparation of N-(3-cyano-4-methyl-1H-indol-7-yl)-4-(morpholinosulfonyl)benzene sulfonamide

To a solution of 4-(N-(3-cyano-4-methyl-1H-indol-7-yl)sulfamoyl)benzenesulfonyl fluoride (25.0 mg, 0.06 mmol) and TEA (19.2 mg, 0.19 mmol) in DCM (2 mL), was added morpholine (6.1 mg, 0.07 mmol). The mixture was stirred for 18 h at room temperature. The mixture was concentrated to afford a crude product, which was purified by column chromatography on silica gel eluting with PE/EA to give N-(3-cyano-4-methyl-1H-indol-7-yl)-4-(morpholino sulfonyl)benzenesulfonamide (Compound 90, 7.8 mg, yield: 28.3%) as a white solid. LCMS (ESI) found: 461 [M+H]⁺. ¹H NMR (400 MHz, DMSO-d6) δ 11.97 (s, 1H), 10.20 (s, 1H), 8.14 (s, 1H), 7.90-7.85 (m, 4H), 6.79 (d, J=7.8, 1H), 6.47 (d, J=7.7, 1H), 3.80-3.53 (m, 4H), 3.07-2.74 (m, 4H), 2.57 (s, 3H).

### Example 56 Preparation of N-(3-cyano-4-methyl-1H-indol-7-yl)-4-((hexahydropyrrolo[1,2-a]pyrazin-2(1H)-yl)sulfonyl)benzenesulfonamide; Compound 91

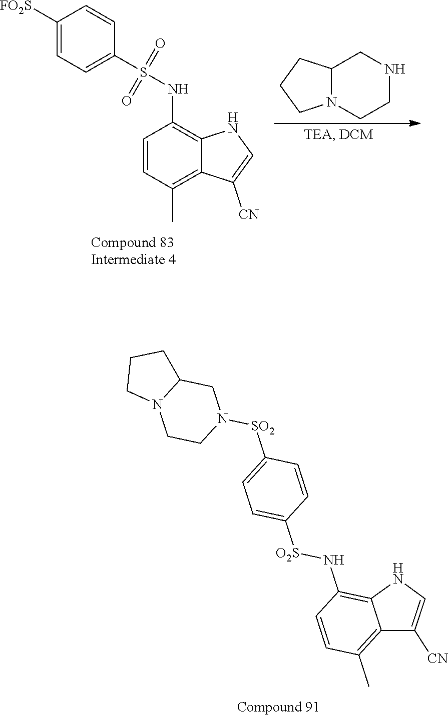

### 1. Preparation of N-(3-cyano-4-methyl-1H-indol-7-yl)-4-((hexahydropyrrolo[1,2-a]pyrazin-2(1H)-yl)sulfonyl)benzenesulfonamide

To a solution of 4-(N-(3-cyano-4-methyl-1H-indol-7-yl)sulfamoyl)benzenesulfonyl fluoride (25.0 mg, 0.06 mmol) and TEA (19.2 mg, 0.19 mmol) in DCM (2 mL) was added octahydropyrrolo[1,2-a]pyrazine (8.8 mg, 0.07 mmol). The mixture was stirred for 18 h at room temperature. The mixture was concentrated to afford a crude product, which was purified by column chromatography on silica gel eluting with PE/EA to give N-(3-cyano-4-methyl-1H-indol-7-yl)-4-((hexahydropyrrolo[1,2-a]pyrazin-2(1H)-yl)sulfonyl)benzenesulfonamide (Compound 91, 6.2 mg, yield: 20.7%) as a white solid. LCMS (ESI) found: 500 [M+H]⁻. ¹H NMR (400 MHz, DMSO-d6) δ 11.86 (s, 1H), 8.07 (s, 1H), 7.86-7.79 (m, 4H), 6.72 (d, J=7.6 Hz, 1H), 6.47 (d, J=7.9 Hz, 1H), 3.68 (d, J=9.6 Hz, 1H), 3.52 (d, J=11.6 Hz, 1H), 2.92 (t, J=10.9 Hz, 2H), 2.52 (s, 3H), 2.28-2.22 (m, 1H), 1.99-1.90 (m, 4H), 1.76-1.73 (m, 1H), 1.63-1.61 (m, 2H), 1.23-1.14 (m, 1H).

### Example 57: Preparation of N-(3-cyano-4-methyl-1H-indol-7-yl)-4-((hexahydropyrrolo[3,4-c] pyrrol-2(1H)-yl)sulfonyl)benzenesulfonamide; Compound 92

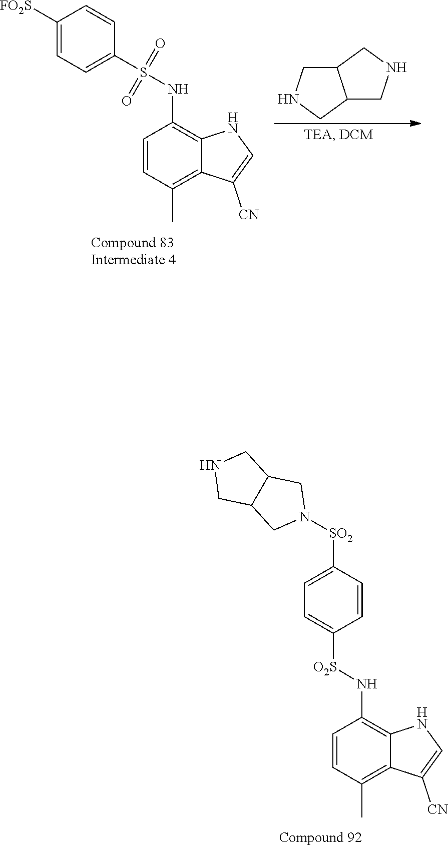

### 1. Preparation of N-(3-cyano-4-methyl-1H-indol-7-yl)-4-((hexahydropyrrolo[3,4-c] pyrrol-2(1H)-yl)sulfonyl)benzenesulfonamide

To a solution of 4-(N-(3-cyano-4-methyl-1H-indol-7-yl)sulfamoyl)benzenesulfonyl fluoride (25.0 mg, 0.06 mmol) and TEA (19.2 mg, 0.19 mmol) in DCM (2 mL) was added octahydropyrrolo[3,4-c]pyrrole (7.2 mg, 0.07 mmol). The mixture was stirred for 18 h at room temperature. The mixture was concentrated to afford a crude product, which was purified by column chromatography on silica gel eluting with PE/EA to give N-(3-cyano-4-methyl-1H-indol-7-yl)-4-((hexahydropyrrolo[3,4-c]pyrrol-2(1H)-yl)sulfonyl)benzenesulfonamide (Compound 92, 9.4 mg, yield: 32.2%) as a white solid. LCMS (ESI) found: 486 [M+H]⁺. ¹H NMR (400 MHz, DMSO-d6) δ 7.97-7.89 (m, 3H), 7.79 (d, J=8.4, 2H), 6.65-6.62 (m, 2H), 3.19-3.16 (m, 4H), 3.04-3.01 (m, 2H), 2.83-2.76 (m, 4H), 2.52 (s, 3H).

### Example 58: Preparation of 4-((2,6-diazaspiro[3.3]heptan-2-yl)sulfonyl)-N-(3-cyano-4-methyl-1H-indol-7-yl)benzenesulfonamide; Compound 93

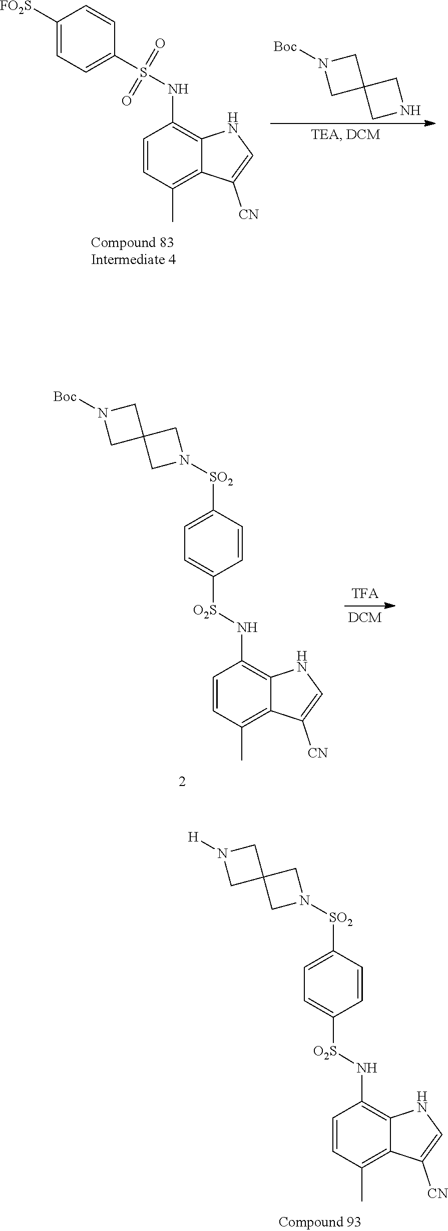

### 1. Preparation of tert-butyl 6-((4-(N-(3-cyano-4-methyl-1H-indol-7-yl)sulfamoyl)phenyl) sulfonyl)-2,6-diazaspiro[3.3]heptane-2-carboxylate

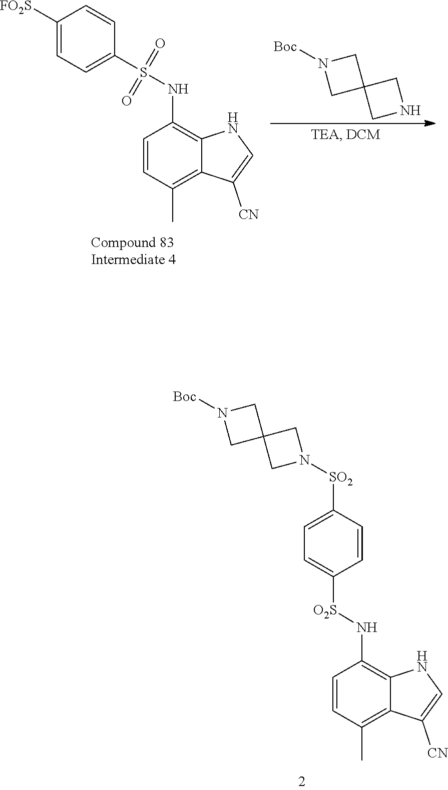

To a solution of 4-(N-(3-cyano-4-methyl-1H-indol-7-yl)sulfamoyl)benzenesulfonyl fluoride (25.0 mg, 0.06 mmol) and TEA (19.2 mg, 0.19 mmol) in DCM (2 mL) was added tert-butyl 2,6-diazaspiro[3.3]heptane-2-carboxylate (13.8 mg, 0.07 mmol). The mixture was stirred for 18 h at room temperature. The mixture was concentrated afford a crude product, which was purified by column chromatography on silica gel eluting with PE/EA to give tert-butyl 6-((4-(N-(3-cyano-4-methyl-1H-indol-7-yl)sulfamoyl)phenyl)sulfonyl)-2,6-diazaspiro[3.3]heptane-2-carboxylate (17.4 mg, yield: 50.1%) as a white solid. LCMS (ESI) found: 572 [M+H]⁺.

### 2. Preparation of 4-((2,6-diazaspiro[3.3]heptan-2-yl)sulfonyl)-N-(3-cyano-4-methyl-1H-indol-7-yl)benzenesulfonamide

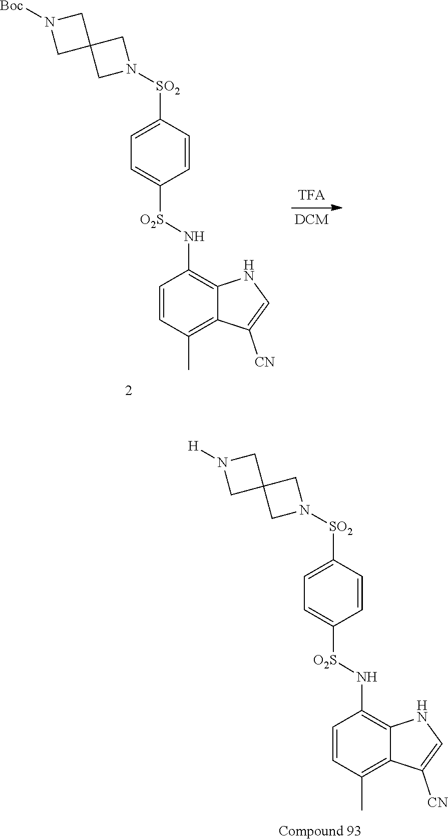 [SMILES: Cc1ccc(NS(=O)(=O)c2ccc(S(=O)(=O)N3CC4(CN(C(=O)OC(C)(C)C)C4)C3)cc2)c2[nH]cc(C#N)c12 ; Cc1ccc(NS(=O)(=O)c2ccc(S(=O)(=O)N3CC4(CNC4)C3)cc2)c2[nH]cc(C#N)c12]

To a solution of tert-butyl 6-((4-(N-(3-cyano-4-methyl-1H-indol-7-yl)sulfamoyl)phenyl) sulfonyl)-2,6-diazaspiro[3.3]heptane-2-carboxylate (17.4 mg, 0.03 mmol) in DCM (2 mL) was added TFA (1 mL). The mixture was stirred at rt for 2 h. The mixture was concentrated. The residue was basified with 10% aqueous NaHCO₃ (20 mL) and extracted with DCM (20 mL×3). The combined organic layer was concentrated to give a crude product, which was purified by prep-HPLC to give 4-((2,6-diazaspiro[3.3]heptan-2-yl)sulfonyl)-N-(3-cyano-4-methyl-1H-indol-7-yl)benzenesulfonamide (Compound 93, 4.7 mg, yield: 33.2%) as a white solid. LCMS (ESI) found: 472 [M+H]⁺. ¹H NMR (400 MHz, DMSO-d6) δ 12.02 (s, 1H), 10.27 (s, 1H), 8.17 (s, 1H), 7.98-7.92 (m, 4H), 6.79 (d, J=7.9 Hz, 1H), 6.44 (d, J=7.7 Hz, 1H), 3.87 (s, 4H), 3.75 (s, 2H), 3.39 (s, 2H), 2.56 (s, 3H).

### Example 59: Preparation of 4-(((1R,5S)-3,6-diazabicyclo[3.1.1]heptan-3-yl)sulfonyl)-N-(3-cyano-4-methyl-1H-indol-7-yl)benzenesulfonamide; Compound 94

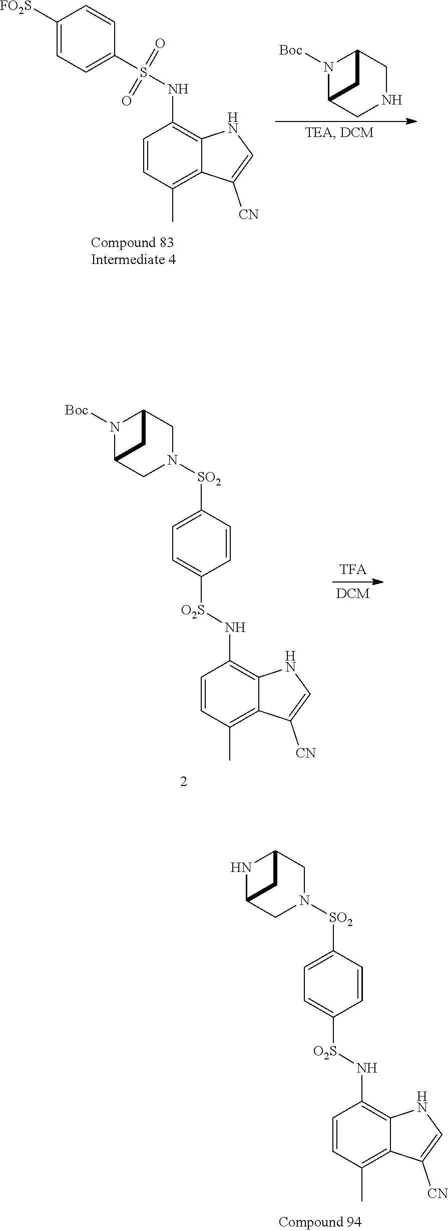

### 1. Preparation of tert-butyl (1R,5S)-3-((4-(N-(3-cyano-4-methyl-1H-indol-7-yl)sulfamoyl) phenyl)sulfonyl)-3,6-diazabicyclo[3.1.1]heptane-6-carboxylate

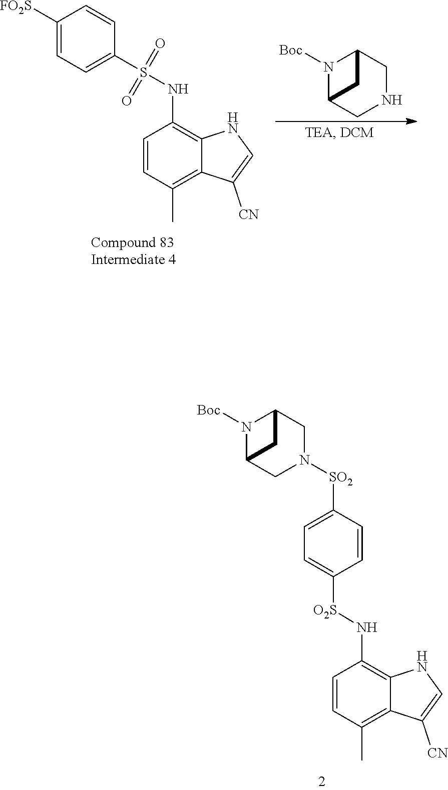

To a solution of 4-(N-(3-cyano-4-methyl-1H-indol-7-yl)sulfamoyl)benzenesulfonyl fluoride (25.0 mg, 0.06 mmol) and TEA (19.2 mg, 0.19 mmol) in DCM (2 mL) was added tert-butyl (1R,5S)-3,6-diazabicyclo[3.1.1]heptane-6-carboxylate (13.8 mg, 0.07 mmol). The mixture was stirred for 18 h at room temperature. The mixture was concentrated to afford a crude product, which was purified by column chromatography on silica gel eluting with PE/EA to give tert-butyl 6-((4-(N-(3-cyano-4-methyl-1H-indol-7-yl)sulfamoyl)phenyl)sulfonyl)-2,6-diazaspiro[3.3]heptane-2-carboxylate (21.4 mg, yield: 62.3%) as a white solid. LCMS (ESI) found: 572 [M+H]⁺.

### 2. Preparation of 4-(((1R,5S)-3,6-diazabicyclo[3.1.1]heptan-3-yl)sulfonyl)-N-(3-cyano-4-methyl-1H-indol-7-yl)benzenesulfonamide

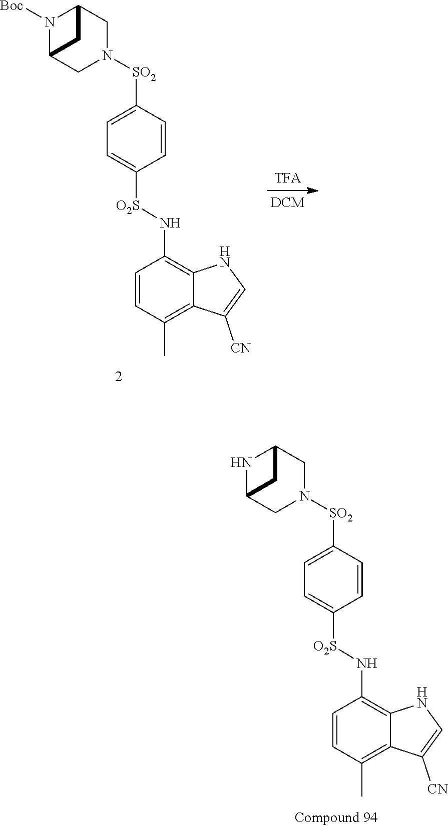 [SMILES: Cc1ccc(NS(=O)(=O)c2ccc(S(=O)(=O)N3C[C@H]4C[C@@H](C3)N4C(=O)OC(C)(C)C)cc2)c2[nH]cc(C#N)c12 ; Cc1ccc(NS(=O)(=O)c2ccc(S(=O)(=O)N3C[C@H]4C[C@@H](C3)N4)cc2)c2[nH]cc(C#N)c12]

To a solution of tert-butyl 6-((4-(N-(3-cyano-4-methyl-1H-indol-7-yl)sulfamoyl)phenyl) sulfonyl)-2,6-diazaspiro[3.3]heptane-2-carboxylate (21.4 mg, 0.04 mmol) in DCM (2 mL) was added TFA (1 mL). The mixture was stirred at rt for 2 h. The mixture was concentrated. The residue was basified with 10% aqueous NaHCO₃ (20 mL) and extracted with DCM (20 Ml×3). The combined organic layer was concentrated to give a crude product, which was purified by prep-HPLC to give 4-(((1R,5S)-3,6-diazabicyclo[3.1.1]heptan-3-yl)sulfonyl)-N-(3-cyano-4-methyl-1H-indol-7-yl)benzenesulfonamide (Compound 94, 7.9 mg, yield: 41.8%) as a white solid. LCMS (ESI) found: 472 [M+H]⁺. ¹H NMR (400 MHz, DMSO-d6) δ 8.05 (s, 1H), 7.93-7.90 (m, 4H), 6.68 (d, J=7.8 Hz, 1H), 6.59 (d, J=7.7 Hz, 1H), 4.37 (s, 1H), 3.79 (s, 1H), 3.31-2.26 (m, 2H), 3.12-2.06 (m, 1H), 2.94-2.90 (m, 2H), 2.73-2.70 (m, 1H), 2.53 (s, 3H), 2.39-2.32 (m, 1H), 1.27 (s, 1H).

### Example 60: Preparation of N-(3-cyano-4-methyl-1H-indol-7-yl)-4-(1H-1,2,3-triazol-1-yl)benzenesulfonamide; Compound 95

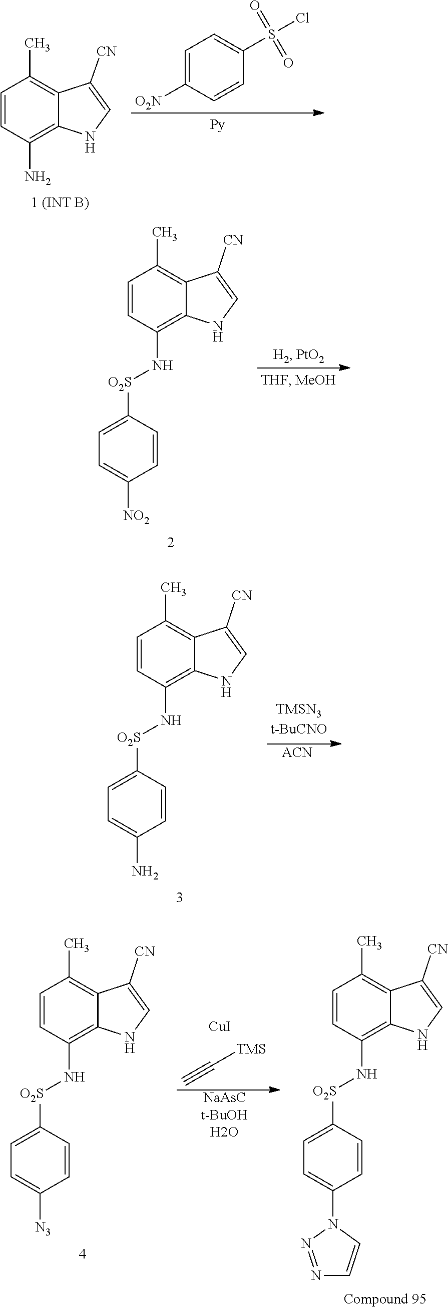

### 1. Preparation of N-(3-cyano-4-methyl-1H-indol-7-yl)-4-nitrobenzenesulfonamide

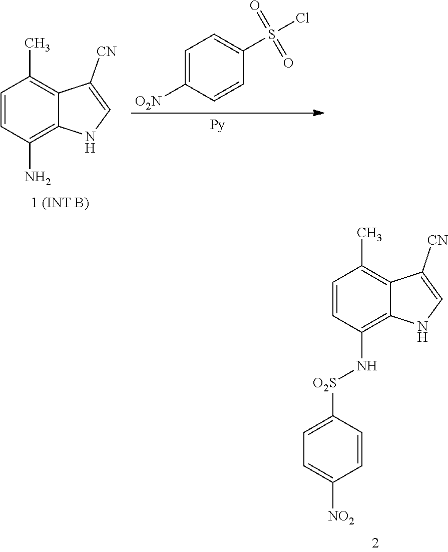 [SMILES: Cc1ccc(N)c2[nH]cc(C#N)c12 ; O=[N+]([O-])c1ccc(S(=O)(=O)Cl)cc1 ; Cc1ccc(NS(=O)(=O)c2ccc([N+](=O)[O-])cc2)c2[nH]cc(C#N)c12]

To a solution of 7-amino-4-methyl-1H-indole-3-carbonitrile (200.0 mg, 1.16 mmol) and pyridine (273.6 mg, 3.50 mmol) in THF (15 mL) was added 4-nitrobenzenesulfonyl chloride (338.3 mg, 1.75 mmol). The mixture was stirred at rt for 18 h. TLC (PE/EA=5:1, R_{f}=0.5) showed the starting material was consumed. The solution was concentrated under reduced pressure to afford a crude product, which was purified by silica gel column chromatography (PE/EA=5:1) to give N-(3-cyano-4-methyl-1H-indol-7-yl)-4-nitro benzenesulfonamide (300.0 mg, yield: 72.1%) as a white solid. LCMS (ESI) found: 357 [M+H]⁺.

### 2. Preparation of 4-amino-N-(3-cyano-4-methyl-1H-indol-7-yl)benzenesulfonamide

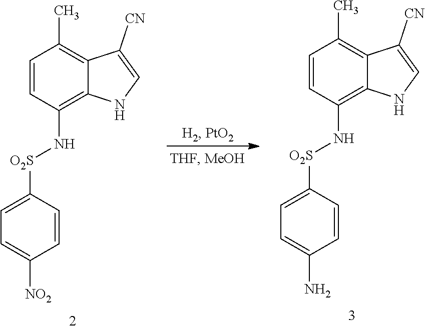 [SMILES: Cc1ccc(NS(=O)(=O)c2ccc([N+](=O)[O-])cc2)c2[nH]cc(C#N)c12 ; Cc1ccc(NS(=O)(=O)c2ccc(N)cc2)c2[nH]cc(C#N)c12]

To a solution of N-(3-cyano-4-methyl-1H-indol-7-yl)-4-nitro benzenesulfonamide (300.0 mg, 0.84 mmol) in THF/CH₃OH (1/1, 10 mL) was added PtO₂ (40.0 mg). The mixture was charged with H₂ for three times and then stirred at rt for 2 h under H₂ atmosphere. LCMS showed the reaction was completed. The mixture was filtered and the filtrate was concentrated under reduced pressure to afford a crude product, which was purified by column chromatography on silica gel eluting with PE/EA from 5/1 to 1/1 to give 4-amino-N-(3-cyano-4-methyl-1H-indol-7-yl)benzenesulfonamide (200.0 mg, yield: 72.8%) as a solid. LCMS (ESI) found: 327 [M+H]⁺.

### 3. Preparation of 4-azido-N-(3-cyano-4-methyl-1H-indol-7-yl)benzene sulfonamide

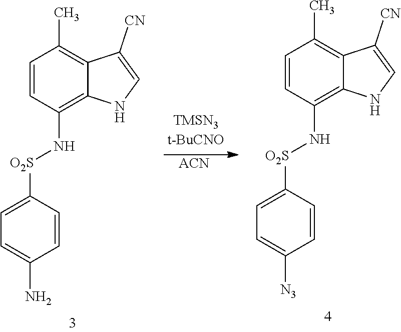 [SMILES: Cc1ccc(NS(=O)(=O)c2ccc(N)cc2)c2[nH]cc(C#N)c12 ; "ACN" ; Cc1ccc(NS(=O)(=O)c2ccc(N=[N+]=[N-])cc2)c2[nH]cc(C#N)c12]

To a solution of 4-amino-N-(3-cyano-4-methyl-1H-indol-7-yl)benzenesulfonamide (200.0 mg, 0.62 mmol) in DCM (5.0 mL) were added t-BuCNO (95.1 mg, 0.92 mmol) and TMSN₃ (106.2 mg, 0.92 mmol) at 0° C. carefully. The mixture was stirred at rt for 18 h under N₂ atmosphere. The mixture was concentrated under reduced pressure and the residue was purified by prep-TLC to give a crude product, which was further purified by prep-HPLC to give 4-azido-N-(3-cyano-4-methyl-1H-indol-7-yl)benzene sulfonamide (150.0 mg, yield: 68.7%) as a white solid. LCMS (ESI) found: 353 [M+H]⁺.

### 4. Preparation of N-(3-cyano-4-methyl-1H-indol-7-yl)-4-(1H-1,2,3-triazol-1-yl)benzene sulfonamide

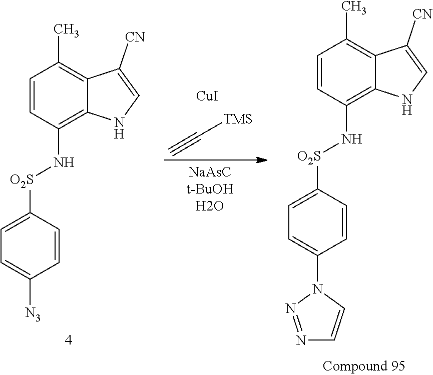

To a solution of 4-azido-N-(3-cyano-4-methyl-1H-indol-7-yl)benzene sulfonamide (25.0 mg, 0.071 mmol) in t-BuOH/H₂O (1/1, 2 mL) were added ethynyltrimethylsilane (13.9 mg, 0.14 mmol), CuI (2.7 mg, 0.014 mmol) and NaAsC (2.7 mg, 0.014 mmol). The mixture was stirred at 100° C. for 18 h under N₂ atmosphere. LCMS showed the reaction was completed. The mixture was concentrated under reduced pressure. The residue was purified by prep-TLC to give a crude product, which was further purified by prep-HPLC to give N-(3-cyano-4-methyl-1H-indol-7-yl)-4-(1H-1,2,3-triazol-1-yl)benzene sulfonamide (Compound 95, 10.0 mg, yield: 44.8%) as a white solid. LCMS (ESI): 379.0 [M+H]⁺. ¹H NMR (400 MHz, DMSO-d6) δ 12.02 (s, 1H), 10.37-9.84 (m, 1H), 8.94 (s, 1H), 8.18 (s, 1H), 8.11 (d, J=8.7 Hz, 2H), 8.03 (d, J=1.1 Hz, 1H), 7.79 (d, J=8.8 Hz, 2H), 6.78 (d, J=7.9 Hz, 1H), 6.57 (d, J=7.7 Hz, 1H), 2.56 (s, 3H).

### Example 61: Preparation of N-(3-cyano-4-methyl-1H-indol-7-yl)-3-(1H-1,2,3-triazol-1-yl)benzenesulfonamide; Compound 96

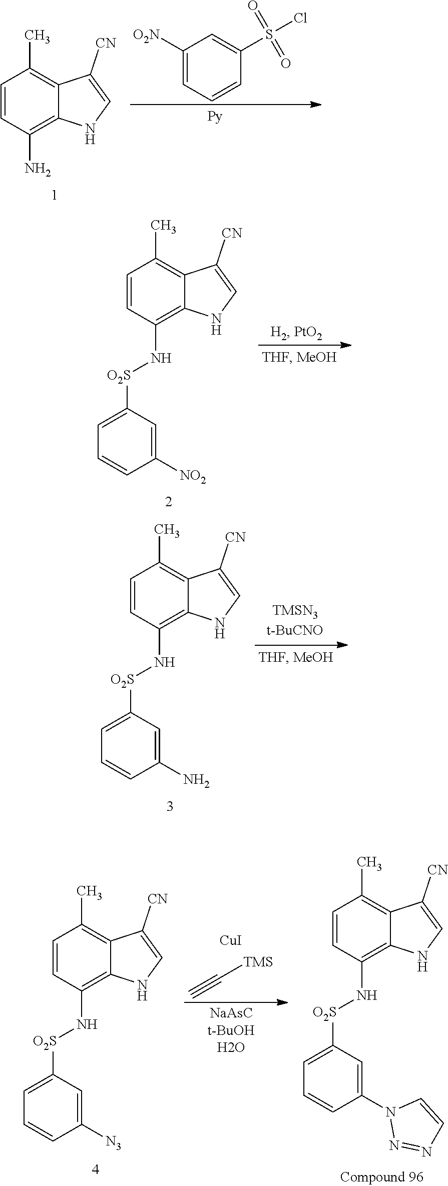

### 1. Preparation of N-(3-cyano-4-methyl-1H-indol-7-yl)-3-nitrobenzenesulfonamide

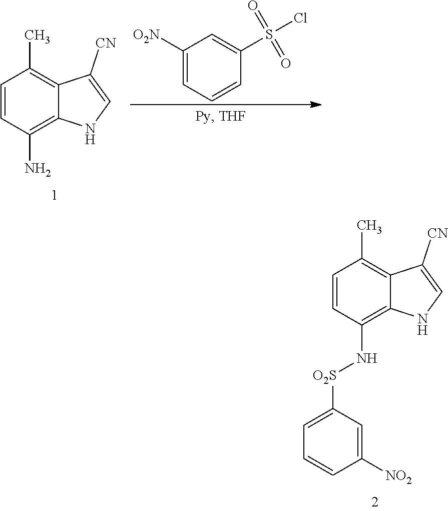 [SMILES: Cc1ccc(N)c2[nH]cc(C#N)c12 ; O=[N+]([O-])c1cccc(S(=O)(=O)Cl)c1 ; Cc1ccc(NS(=O)(=O)c2cccc([N+](=O)[O-])c2)c2[nH]cc(C#N)c12]

To a solution of 7-amino-4-methyl-1H-indole-3-carbonitrile (200.0 mg, 1.16 mmol) and pyridine (273.6 mg, 3.50 mol) in THF (15 mL) was added 3-nitrobenzenesulfonyl chloride (338.3 mg, 1.75 mmol). The mixture was stirred for 18 h at room temperature. TLC (PE/EA=5:1, R_{f}=0.5) showed the starting material was consumed. The solution was concentrated under reduced pressure to afford a crude product, which was purified by column chromatography on silica gel eluting with PE/EA from 15/1 to 5/1 to give N-(3-cyano-4-methyl-1H-indol-7-yl)-3-nitrobenzenesulfonamide (315.0 mg, yield: 75.9%) as a white solid. LCMS (ESI) found: 357 [M+H]⁺.

### 2. Preparation of 3-amino-N-(3-cyano-4-methyl-1H-indol-7-yl)benzenesulfonamide

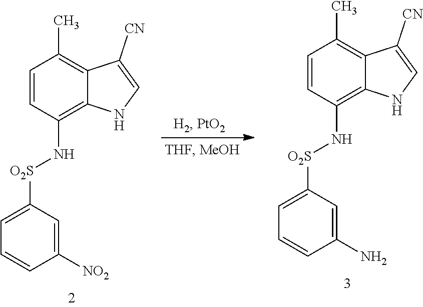 [SMILES: Cc1ccc(NS(=O)(=O)c2cccc([N+](=O)[O-])c2)c2[nH]cc(C#N)c12 ; Cc1ccc(NS(=O)(=O)c2cccc(N)c2)c2[nH]cc(C#N)c12]

To a solution of N-(3-cyano-4-methyl-1H-indol-7-yl)-3-nitrobenzenesulfonamide (300.0 mg, 0.84 mmol) in THF/CH₃OH (1/1, 10 mL) was added PtO₂ (40.0 mg). The mixture was charged with H₂ for three times and then stirred at rt for 2 h under H₂ atmosphere. LCMS showed the reaction was completed. The mixture was filtered and concentrated under reduced pressure to afford a crude product, which was purified by column chromatography on silica gel eluting with PE/EA from 5/1 to 1/1 to give 3-amino-N-(3-cyano-4-methyl-1H-indol-7-yl)benzenesulfonamide (223.0 mg, yield: 80.9%) as a solid. LCMS (ESI) found: 327 [M+H]⁺.

### 3. Preparation of 3-azido-N-(3-cyano-4-methyl-1H-indol-7-yl)benzenesulfonamide

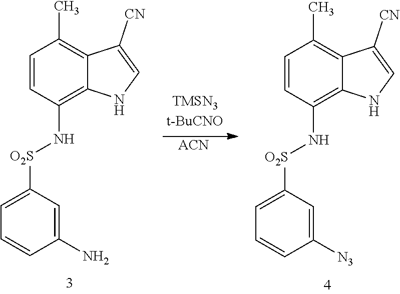 [SMILES: Cc1ccc(NS(=O)(=O)c2cccc(N)c2)c2[nH]cc(C#N)c12 ; "ACN" ; Cc1ccc(NS(=O)(=O)c2cccc(N=[N+]=[N-])c2)c2[nH]cc(C#N)c12]

To a solution of 3-amino-N-(3-cyano-4-methyl-1H-indol-7-yl)benzenesulfonamide (200.0 mg, 0.62 mmol) in MeCN (5 mL) were added t-BuCNO (95.0 mg, 0.92 mmol) and TMSN₃ (106.0 mg, 0.92 mmol) at 0° C. carefully. The mixture was stirred at rt for 18 h under N₂ atmosphere. LCMS showed the reaction was completed. The solvent was concentrated under reduced pressure. The residue was purified by prep-TLC to give a crude product, which was further purified by prep-HPLC to give 3-azido-N-(3-cyano-4-methyl-1H-indol-7-yl)benzene sulfonamide (163.2 mg, yield: 74.2%) as a white solid. LCMS (ESI) found: 353 [M+H]⁺.

### 4. Preparation of N-(3-cyano-4-methyl-1H-indol-7-yl)-3-(1H-1,2,3-triazol-1-yl)benzene sulfonamide

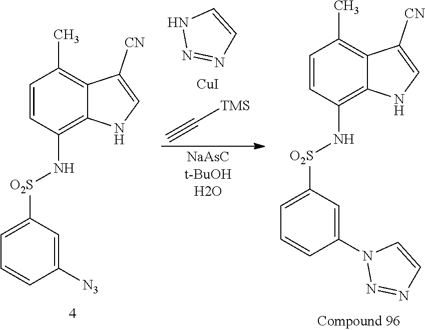

To a solution of 3-azido-N-(3-cyano-4-methyl-1H-indol-7-yl)benzene sulfonamide (75.0 mg, 0.21 mmol) in t-BuOH/H₂O (1/1, 2 mL) were added ethynyltrimethylsilane (41.7 mg, 0.43 mmol), CuI (8.1 mg, 0.042 mmol) and NaAsC (8.1 mg, 0.042 mmol). The mixture was stirred at 100° C. for 18 h under N₂ atmosphere. LCMS showed the reaction was completed. The mixture was concentrated under reduced pressure. The residue was purified by prep-TLC to give a crude product, which was further purified by prep-HPLC to give N-(3-cyano-4-methyl-1H-indol-7-yl)-3-(1H-1,2,3-triazol-1-yl)benzene sulfonamide (Compound 96, 38.7 mg, yield: 48.6%) as a white solid. LCMS (ESI) found: 379 [M+H]⁺. ¹H NMR (400 MHz, DMSO-d6) δ 12.08 (s, 1H), 10.18 (s, 1H), 8.96 (d, J=0.9 Hz, 1H), 8.37 (t, J=1.7 Hz, 1H), 8.28-8.17 (m, 2H), 8.08 (d, J=1.0 Hz, 1H), 7.85-7.73 (m, 2H), 6.84 (d, J=8.1 Hz, 1H), 6.64 (d, J=7.7 Hz, 1H), 2.61 (s, 3H).

### Example 62: Preparation of N-(3-cyano-4-methyl-1H-indol-7-yl)-3-(4-(2-methoxyethyl)-1H-1,2,3-triazol-1-yl)benzenesulfonamide; Compound 97

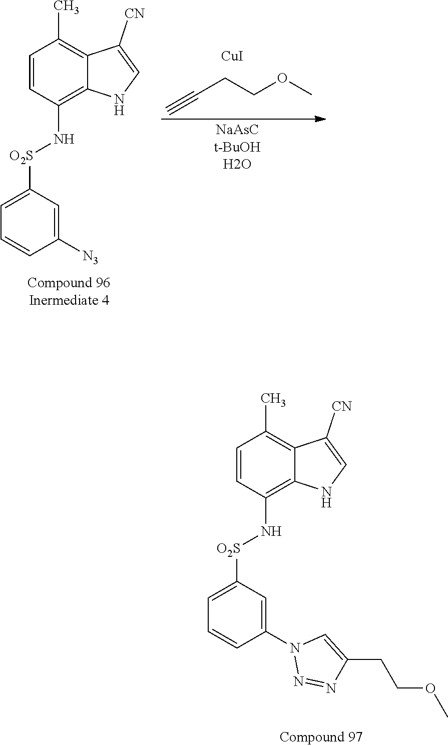

### 1. Preparation of N-(3-cyano-4-methyl-1H-indol-7-yl)-3-(4-(2-methoxyethyl)-1H-1,2,3-triazol-1-yl)benzenesulfonamide

To a solution of 3-azido-N-(3-cyano-4-methyl-1H-indol-7-yl)benzene sulfonamide (100.0 mg, 0.28 mmol) in t-BuOH/H₂O (1/1, 4 mL) were added 4-methoxybut-1-yne (47.6 mg, 0.056 mmol), CuI (10.8 mg, 0.056 mmol) and NaAsC (10.8 mg, 0.056 mmol). The mixture was charged with N₂ and stirred at 100° C. for 18 h under N₂ atmosphere. LCMS showed the reaction was completed. The mixture was and concentrated under reduced pressure. The residue was purified by prep-TLC to give a crude product, which was further purified by prep-HPLC to give N-(3-cyano-4-methyl-1H-indol-7-yl)-3-(4-(2-methoxyethyl)-1H-1,2,3-triazol-1-yl)benzenesulfonamide (Compound 97, 68.3 mg, yield: 53.6%) as a white solid. LCMS (ESI) found: 436 [M+H]⁺. ¹H NMR (400 MHz, DMSO-d6) δ 12.05 (s, 1H), 10.13 (s, 1H), 8.67 (s, 1H), 8.28 (s, 1H), 8.19 (d, J=3.1 Hz, 1H), 8.17-8.11 (m, 1H), 7.77-7.66 (m, 2H), 6.78 (d, J=7.7 Hz, 1H), 6.56 (d, J=7.7 Hz, 1H), 3.65 (t, J=6.6 Hz, 2H), 3.28 (s, 3H), 2.96 (t, J=6.6 Hz, 2H), 2.56 (s, 3H).

### Example 63: Preparation of 3-cyano-N-(3-cyano-4-methyl-1H-indol-7-yl)-5-fluorobenzenesulfonamide; Compound 98

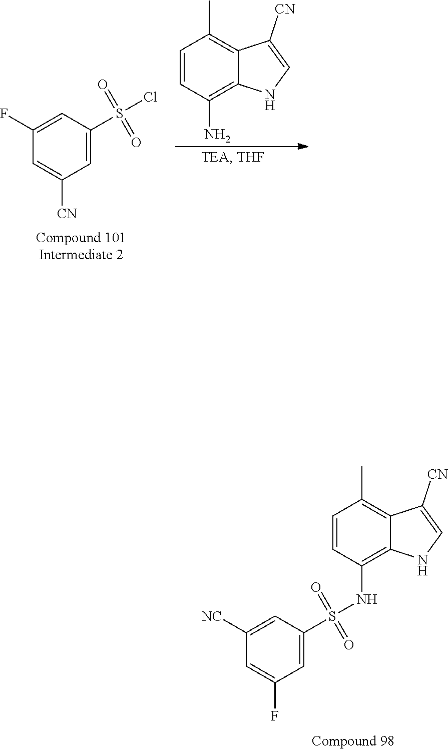

### 1. Preparation of 3-cyano-N-(3-cyano-4-methyl-1H-indol-7-yl)-5-fluorobenzenesulfonamide

To a solution of 7-amino-4-methyl-1H-indole-3-carbonitrile (46.0 mg, 0.27 mmol) and TEA (103.0 mg, 1.02 mmol) in THE (5 mL) was added 3-cyano-5-fluorobenzenesulfonyl chloride (59.1 mg, 0.27 mmol). The reaction mixture was stirred at rt for 18 h. LCMS showed the reaction was completed. The mixture was concentrated under reduced pressure. The residue was purified by prep-TLC to give a crude product, which was further purified by prep-HPLC to give 3-cyano-N-(3-cyano-4-methyl-1H-indol-7-yl)-5-fluorobenzenesulfonamide (Compound 98, 59.6 mg, yield: 62.6%) as a white solid. LCMS (ESI) found: 355 [M+H]⁺. ¹H NMR (400 MHz, DMSO-d6) δ 12.09 (s, 1H), 10.24 (s, 1H), 8.27-8.21 (m, 2H), 7.96 (s, 1H), 7.83-7.81 (m, 1H), 6.83 (d, J=8.0 Hz, 1H), 6.56 (d, J=8.0 Hz, 1H), 2.59 (s, 3H).

### Example 64: 4-azido-N-(3-cyano-4-methyl-1H-indol-7-yl)benzenesulfonamide; Compound 99

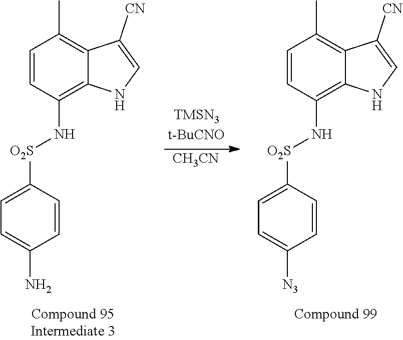 [SMILES: Cc1ccc(NS(=O)(=O)c2ccc(N)cc2)c2[nH]cc(C#N)c12 ; "CH3CN" ; Cc1ccc(NS(=O)(=O)c2ccc(N=[N+]=[N-])cc2)c2[nH]cc(C#N)c12]

To a solution of 4-amino-N-(3-cyano-4-methyl-1H-indol-7-yl)benzenesulfonamide (200 mg, 0.62 mmol) in CH₃CN (5 mL) were added t-BuCNO (95.0 mg, 0.92 mmol) and TMSN₃ (106.0 mg, 0.92 mmol) at 0° C. The mixture was stirred at rt for 18 h. LCMS showed the reaction was completed. The mixture was concentrated. The residue was purified by prep-TLC to give a crude product, which was further purified by prep-HPLC to give 4-azido-N-(3-cyano-4-methyl-1H-indol-7-yl) benzenesulfonamide (150.0 mg, yield: 68.7%) as a white solid. LCMS (ESI): 353 [M+H]⁺. ¹H NMR (400 MHz, DMSO-d6) δ 11.93 (s, 1H), 9.92 (s, 1H), 8.17 (s, 1H), 7.72-7.65 (m, 2H), 7.27-7.21 (m, 2H), 6.79 (d, J=8.0 Hz, 1H), 6.56 (d, J=1.0 Hz, 1H), 2.57 (s, 3H).

### Example 65: Preparation of 3-cyano-N-(3-(difluoromethyl)-4-methyl-1H-indol-7-yl)benzenesulfonamide; Compound 100

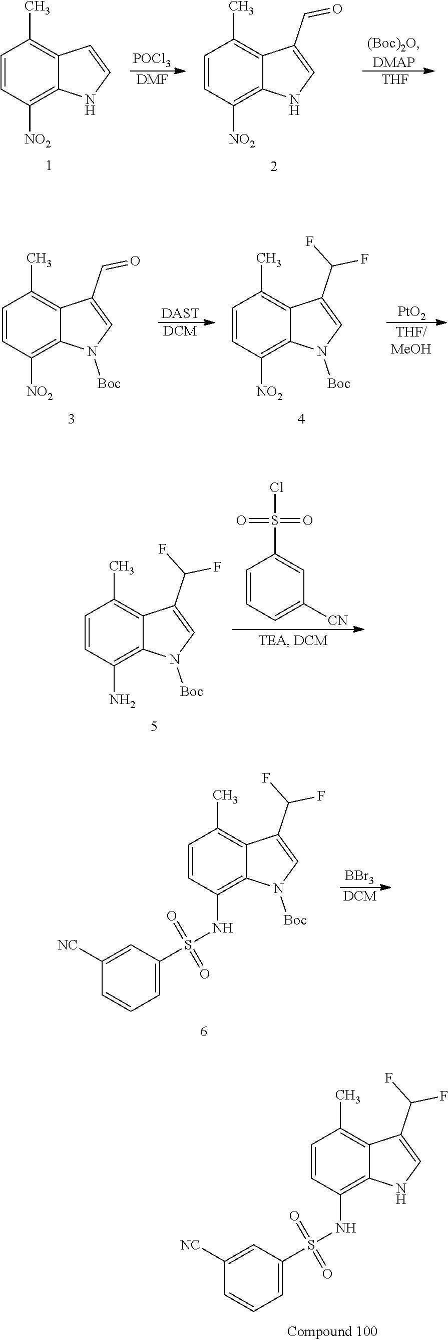

### 1. Preparation of 4-methyl-7-nitro-1H-indole-3-carbaldehyde

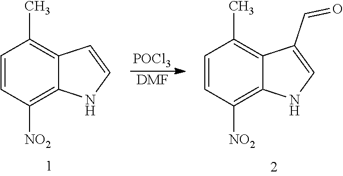

POCl₃ (1.5 mL, 16.09 mmol) was added dropwise to DMF (12 mL) at 0° C. and stirred for 30 min to give a solution. To the solution was added 4-methyl-7-nitro-1H-indole (2.0 g, 11.35 mmol) in DMF (20 mL). The mixture was stirred at 60° C. for 3 h. TLC showed the starting material was consumed and a new spot was detected. The reaction mixture was poured into aqueous NaHCO₃ (50 mL) and extracted with EA (50 mL×3). The combined organic layer was washed with brine (20 mL×3), dried over anhydrous Na₂SO₄, filtered and concentrated to afford a crude product, which was triturated with MTBE: Hexane (1/1) to give 4-methyl-7-nitro-1H-indole-3-carbaldehyde (1.6 g, yield: 69.0%) as a yellow solid. LCMS (ESI) found: 205 [M+H]⁺.

### 2. Preparation of tert-butyl 3-formyl-4-methyl-7-nitro-1H-indole-1-carboxylate

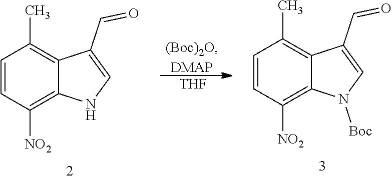

To a solution of 4-methyl-7-nitro-1H-indole-3-carbaldehyde (600.0 mg, 2.94 mmol) in THF (15 mL) were added Boc₂O (1.28 g, 5.88 mmol) and DMAP (360.0 mg, 2.94 mmol). The mixture was stirred at rt for 18 h. TLC (PE/EA=3:1, R_{f}=0.5) showed the starting material was consumed. The mixture was concentrated under reduced pressure to afford a crude product, which was purified by column chromatography on silica gel eluting with PE/EA from 15/1 to 5:1 to give tert-butyl 3-formyl-4-methyl-7-nitro-1H-indole-1-carboxylate (520.0 mg, yield: 58.2%) as a white solid. LCMS (ESI) found: 305 [M+H]⁺. ¹H NMR (400 MHz, DMSO-d6) δ 9.73 (s, 1H), 8.03 (d, 1H), 7.55 (d, 1H), 7.12 (s, 1H), 2.57 (s, 3H), 1.63 (s, 9H).

### 3. Preparation of tert-butyl 3-(difluoromethyl)-4-methyl-7-nitro-1H-indole-1-carboxylate

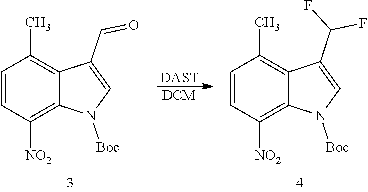

A solution of tert-butyl 3-formyl-4-methyl-7-nitro-1H-indole-1-carboxylate (520.0 mg, 1.72 mmol) in dry DCM (10 mL) was added DAST (2.7 g, 6.86 mmol) at −5° C. The mixture was stirred at rt for 18 h. TLC (PE/EA=5:1, R_{f}=0.5) showed the starting material was consumed. The mixture was filtered through celite and the filtrate was concentrated under reduced pressure to give a crude product, which was purified by column chromatography on silica gel eluting with PE/EA from 30/1 to 15:1 to give tert-butyl 3-(difluoromethyl)-4-methyl-7-nitro-1H-indole-1-carboxylate (475.0 mg, yield: 85.1%) as a white solid. LCMS (ESI) found: 327 [M+H]⁺. ¹H NMR (400 MHz, DMSO-d6) δ 8.03 (d, 1H), 7.55 (d, 1H), 7.12 (s, 1H), 6.44 (t, 3H), 2.57 (s, 3H), 1.63 (s, 9H).

### 4. Preparation of tert-butyl 7-amino-3-(difluoromethyl)-4-methyl-1H-indole-1-carboxylate

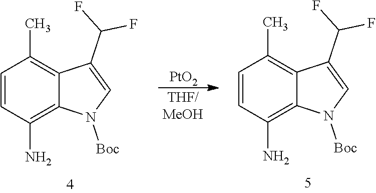

To a solution of tert-butyl 3-(difluoromethyl)-4-methyl-7-nitro-1H-indole-1-carboxylate (470.0 mg, 1.44 mmol) in THF/CH₃OH (1/1, 10 mL) was added PtO₂ (47 mg). The mixture was evaporated and backfilled with H₂ for three times. The mixture was stirred at rt for 2 h under H₂ atmosphere. LCMS showed the reaction was completed. The mixture was filtered and the filtrate was concentrated under reduced pressure to afford a crude product, which was purified by column chromatography on silica gel eluting with PE/EA from 5/1 to 1/1 to give tert-butyl 7-amino-3-(difluoromethyl)-4-methyl-1H-indole-1-carboxylate (400.0 mg, yield: 93.7%) as a white solid. LCMS (ESI) found: 327 [M+H]⁺.

### 5. Preparation of tert-butyl 7-((3-cyanophenyl)sulfonamido)-3-(difluoromethyl)-4-methyl-1H-indole-1-carboxylate

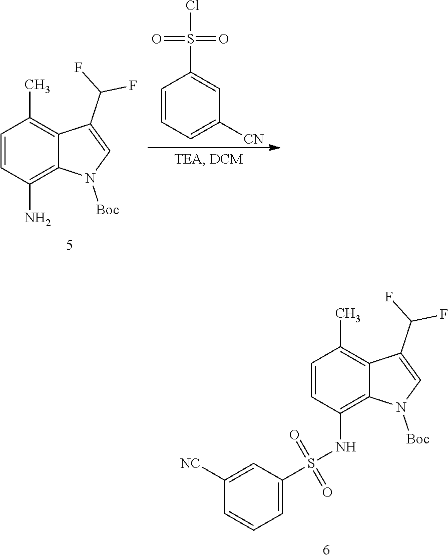

To a solution of tert-butyl 7-amino-3-(difluoromethyl)-4-methyl-1H-indole-1-carboxylate (10.0 mg, 0.085 mmol) and TEA (17.17 mg, 0.17 mmol) in DCM (5 mL) was added 3-cyanobenzenesulfonyl chloride (25.0 mg, 0.085 mmol). The mixture was stirred at rt for 18 h. LCMS showed the reaction was completed. The mixture was concentrated under reduced pressure. The residue was purified by prep-TLC to give a crude product, which was further purified by prep-HPLC to give tert-butyl 7-((3-cyanophenyl)sulfonamido)-3-(difluoromethyl)-4-methyl-1H-indole-1-carboxylate (30.0 mg, yield: 76.2%) as a white solid. LCMS (ESI) found: 462 [M+H]⁺. ¹H NMR (400 MHz, DMSO-d6) δ 10.01 (s, 1H), 8.27 (d, 1H), 8.19 (s, 1H), 8.11 (d, 1H), 8.03 (d, 1H), 7.78 (t, 1H), 7.55 (d, 1H), 7.12 (s, 1H), 6.44 (t, 3H), 2.57 (s, 3H), 1.63 (s, 9H).

### 6. Preparation of 3-cyano-N-(3-(difluoromethyl)-4-methyl-1H-indol-7-yl)benzenesulfonamide

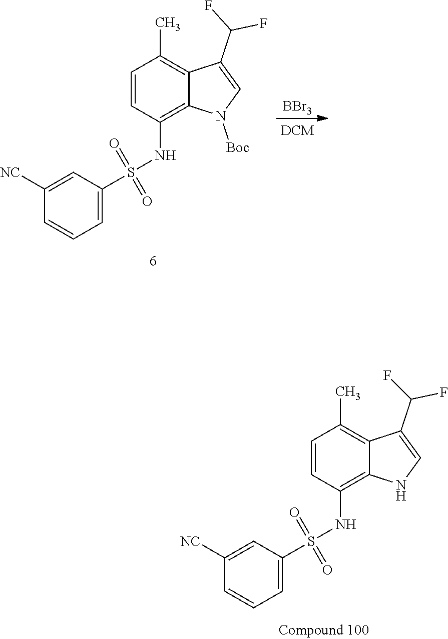

To a solution of tert-butyl 7-((3-cyanophenyl)sulfonamido)-3-(difluoromethyl)-4-methyl-1H-indole-1-carboxylate (30.0 mg, 0.065 mmol) in DCM (5 mL) was added BBr₃ (16.3 mg, 0.065 mmol) at 0° C. The mixture was stirred at rt for 1 h under N₂ atmosphere. LCMS showed the reaction was completed. The mixture was concentrated under reduced pressure. The residue was purified by prep-TLC to give a crude product, which was further purified by prep-HPLC to give 3-cyano-N-(3-(difluoromethyl)-4-methyl-1H-indol-7-yl)benzenesulfonamide (Compound 100, 6.4 mg, yield: 27.2%) as a white solid. LCMS (ESI) found: 362 [M+H]⁺. ¹H NMR (400 MHz, DMSO-d6) δ 11.33 (s, 1H), 10.05 (s, 1H), 8.18-8.07 (m, 2H), 7.99-7.90 (m, 1H), 7.74 (t, J=8.0 Hz, 1H), 7.69 (d, J=2.5 Hz, 1H), 7.23 (t, J=55.8 Hz, 1H), 6.74 (d, J=8.3 Hz, 1H), 6.51 (d, J=7.7 Hz, 1H), 2.49 (s, 3H).

### Example 66: Preparation of 3-cyano-N-(3-(difluoromethyl)-4-methyl-1H-indol-7-yl)-5-fluorobenzene sulfonamide; Compound 101

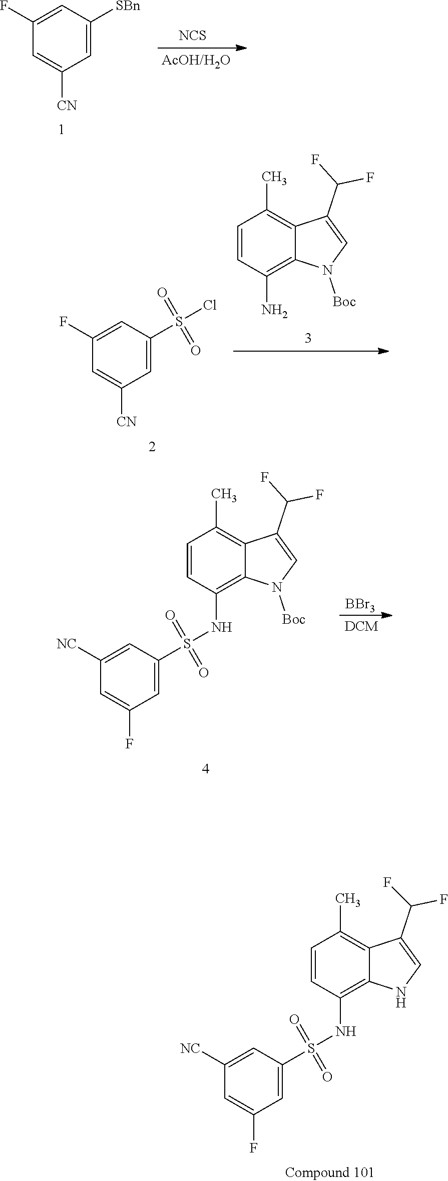

### 1. Preparation of 3-cyano-5-fluorobenzenesulfonyl chloride

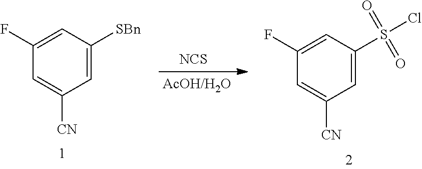

To a solution of 3-(benzylthio)-5-fluorobenzonitrile (500.0 mg, 2.08 mol) in AcOH/H₂O (9/1, 5 mL) was added NCS (825.0 mg, 6.12 mol). The mixture was stirred at rt for 2 h. TLC (PE/EA=3:1, R_{f}=0.5) showed the starting material was consumed. The mixture was filtered through celite and the filtrate was concentrated under reduced pressure to give a crude product, which was purified by column chromatography on silica gel eluting with PE/EA from 15/1 to 5:1 to give 3-cyano-5-fluorobenzenesulfonyl chloride (250.0 mg, yield: 54.7%) as a white solid. ¹H NMR (400 MHz, DMSO-d6) δ 8.07 (s, 1H), 7.96 (s, 1H), 7.56 (s, 1H).

### 2. Preparation of tert-butyl 7-((3-cyano-5-fluorophenyl)sulfonamido)-3-(difluoromethyl)-4-methyl-1H-indole-1-carboxylate

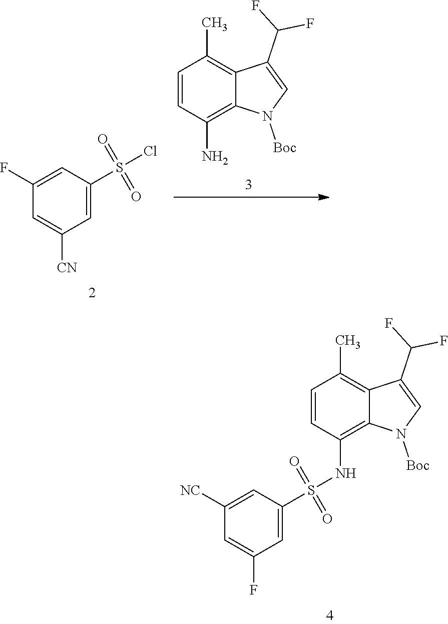 [SMILES: N#Cc1cc(F)cc(S(=O)(=O)Cl)c1 ; Cc1ccc(N)c2c1c(C(F)F)cn2C(=O)OC(C)(C)C ; Cc1ccc(NS(=O)(=O)c2cc(F)cc(C#N)c2)c2c1c(C(F)F)cn2C(=O)OC(C)(C)C]

To a solution of tert-butyl 7-amino-3-(difluoromethyl)-4-methyl-1H-indole-1-carboxylate (100.0 mg, 0.34 mmol) and TEA (103.0 mg, 1.02 mmol) in THF (5 mL) was added 3-cyano-5-fluorobenzenesulfonyl chloride (74.8 mg, 0.34 mmol). The reaction mixture was stirred at rt for 18 h. LCMS showed the reaction was completed. The mixture was concentrated under reduced pressure. The residue was purified by prep-TLC to give a crude product, which was further purified by prep-HPLC to give tert-butyl 7-((3-cyano-5-fluoro phenyl)sulfonamido)-3-(difluoromethyl)-4-methyl-1H-indole-1-carboxy late (103.7 mg, yield: 63.5%) as a white solid. LCMS (ESI) found: 480 [M+H]⁺.

### 3. Preparation of 3-cyano-N-(3-(difluoromethyl)-4-methyl-1H-indol-7-yl)-5-fluorobenzene sulfonamide

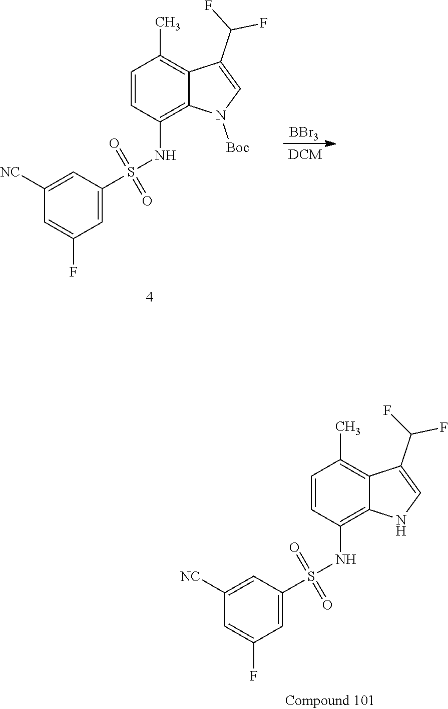

To a solution of tert-butyl 7-((3-cyano-5-fluorophenyl) sulfonamido)-3-(difluoromethyl)-4-methyl-1H-indole-1-carboxylate (100.0 mg, 0.21 mmol) in DCM (15 mL) was added BBr₃ (52.1 mg, 0.21 mmol). The mixture was stirred at rt for 1 h. LCMS showed the reaction was completed. The mixture was concentrated under reduced pressure. The residue was purified by prep-TLC to give a crude product, which was further purified by prep-HPLC to give 3-cyano-N-(3-(difluoromethyl)-4-methyl-1H-indol-7-yl)-5-fluorobenzene sulfonamide (Compound 101, 30.0 mg, yield: 30.8%) as a white solid. LCMS (ESI): 380 [M+H]⁺. ¹H NMR (400 MHz, DMSO-d6) δ 11.39 (s, 1H), 10.15 (s, 1H), 8.25-8.16 (m, 1H), 7.97 (d, J=1.3 Hz, 1H), 7.87-7.78 (m, 1H), 7.70 (d, J=2.4 Hz, 1H), 7.23 (t, J=55.8 Hz, 1H), 6.76 (d, J=7.8 Hz, 1H), 6.59-6.49 (m, 1H), 2.49 (s, 3H).

### Example 67: Preparation of N-(3-chloro-4-iodo-1H-indol-7-yl)-3-cyanobenzenesulfonamide; Compound 102

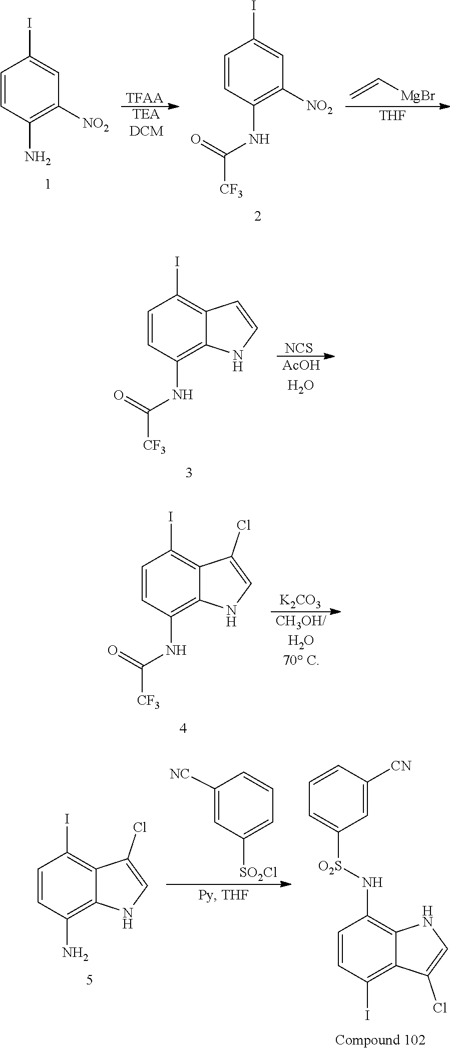

### 1. Preparation of 2,2,2-trifluoro-N-(4-iodo-2-nitrophenyl)acetamide

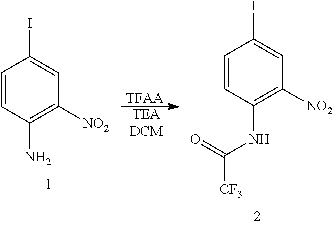 [SMILES: Nc1ccc(I)cc1[N+](=O)[O-] ; "TFAA" ; "TEADCM" ; O=C(Nc1ccc(I)cc1[N+](=O)[O-])C(F)(F)F]

To a solution of 4-iodo-2-nitroaniline (25.0 g, 94.69 mmol) in DCM (250 mL) were added TEA (28.7 g, 284.07 mmol) and TFAA (39.8 g, 189.38 mmol) at 0° C. The mixture was stirred for 1 h at 0° C. After completion, the reaction mixture was quenched with aqueous NaHCO₃ (500 mL) and extracted with DCM (500 mL×3). The combined organic layer was dried over MgSO₄, filtered and concentrated to give a crude product, which was purified by flash to give 2,2,2-trifluoro-N-(4-iodo-2-nitrophenyl)acetamide (27.5 g, yield: 80.7%) as a yellow solid. LCMS (ESI): 361 [M+H]⁺.

### 2. Preparation of 2,2,2-trifluoro-N-(4-iodo-1H-indol-7-yl)acetamide

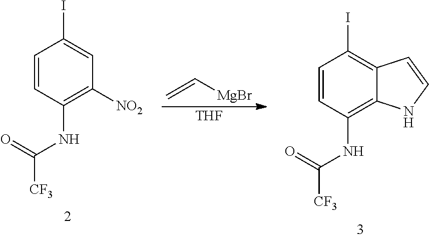

To a solution of 2,2,2-trifluoro-N-(4-iodo-2-nitrophenyl)acetamide (5.3 g, 14.72 mmol) in THF (200 mL) was added vinylmagnesium bromide (73.6 mL, 73.60 mmol) at 0° C. slowly. The mixture was stirred for 2 h at 0° C. until the starting material was consumed completely. The mixture was quenched with aqueous NH₄Cl (200 mL) and extracted with EA (250 mL×3). The combine organic layer was dried over MgSO₄, filtered and concentrated to give a crude product, which was purified by flash to give 2,2,2-trifluoro-N-(4-iodo-1H-indol-7-yl) acetamide (1.7 g, yield: 32.6%) as a yellow solid. LCMS (ESI): 355 [M+H]⁺.

### 3. Preparation of N-(3-chloro-4-iodo-1H-indol-7-yl)-2,2,2-trifluoroacetamide

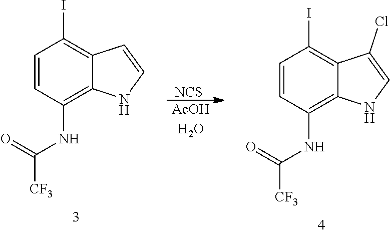

To a solution of 2,2,2-trifluoro-N-(4-iodo-1H-indol-7-yl)acetamide (1.7 g, 4.80 mmol) in AcOH/H₂O (9/1, 10 mL) was added NCS (1.4 g, 24.0 mmol). The mixture was stirred at rt for 2 h. The reaction was quenched with aqueous Na₂S₂₀₃ (15 mL) and extracted with DCM (15 mL×3). The combined organic layer was concentrated to give a crude product, which was purified by column chromatography on silica gel eluting with PE/EA from 15/1 to 5/1 to afford N-(3-chloro-4-iodo-1H-indol-7-yl)-2,2,2-trifluoroacetamide (1.2 g, 3.09 mmol, yield: 64.35%) as a white solid. LCMS (ESI): 389 [M+H]⁺.

### 4. Preparation of 3-chloro-4-iodo-1H-indol-7-amine

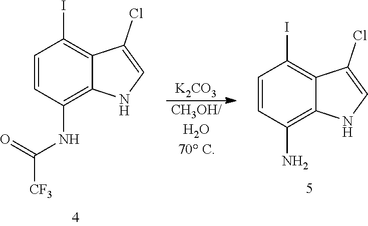

To a solution of N-(3-chloro-4-iodo-1H-indol-7-yl)-2,2,2-trifluoroacetamide (1.0 g, 2.57 mmol) in CH₃OH/H₂O (4/1, 10 mL) was added K₂CO₃ (1.8 g, 12.85 mmol). The mixture was stirred at 70° C. for 2 h until the starting material was consumed completely. The mixture was cooled to room temperature, the solid was filtered and the filtrate was concentrated to give a crude product, which was purified by flash to give 3-chloro-4-iodo-1H-indol-7-amine (500.0 mg, yield: 66.5%) as a white solid. LCMS (ESI): 293 [M+H]⁺.

### 5. Preparation of N-(3-chloro-4-iodo-1H-indol-7-yl)-3-cyanobenzenesulfonamide

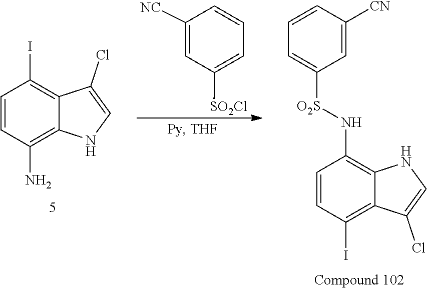

To a solution of 3-chloro-4-iodo-1H-indol-7-amine (500.0 mg, 1.71 mmol) and pyridine (270.2 mg, 3.42 mmol) in THF (3 mL) was added 3-cyanobenzene-1-sulfonyl chloride (689.5 mg, 3.42 mmol). The mixture was stirred at rt for 2 h. The mixture was concentrated to give a crude product, which was purified by column chromatography on silica gel eluting with PE/EA from 15/1 to 5/1 to afford N-(3-chloro-4-iodo-1H-indol-7-yl)-3-cyanobenzene sulfonamide (Compound 102, 370.0 mg, yield: 47.3%) as a white solid. LCMS (ESI): 458 [M+H]⁺. ¹H NMR (400 MHz, DMSO-d6) δ 11.42 (s, 1H), 10.19 (s, 1H), 8.19-8.17 (m, 1H), 8.14-8.09 (m, 1H), 7.98-7.93 (m, 1H), 7.74 (t, J=7.9 Hz, 1H), 7.60 (d, J=2.8 Hz, 1H), 7.40 (d, J=8.0 Hz, 1H), 6.44 (d, J=8.0 Hz, 1H).

### Example 68: Preparation of N-(3-chloro-5-methoxy-1H-indol-7-yl)-3-cyanobenzenesulfonamide; Compound 103

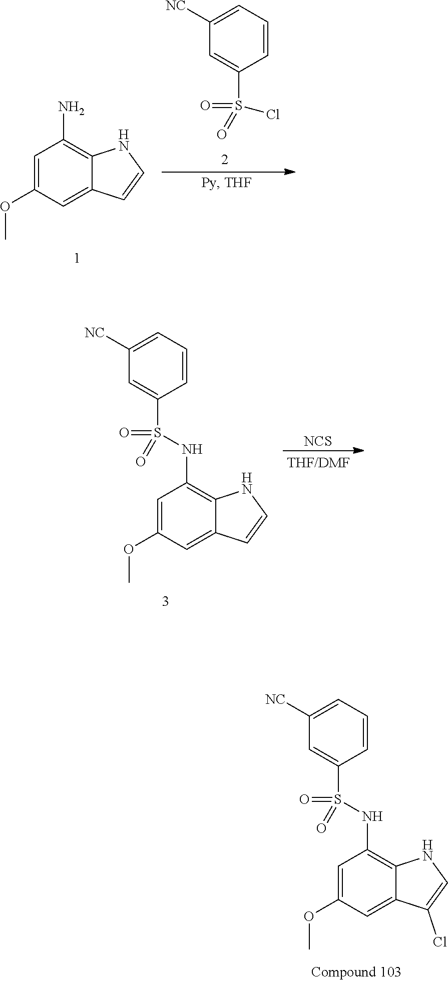 [SMILES: COc1cc(N)c2[nH]ccc2c1 ; N#Cc1cccc(S(=O)(=O)Cl)c1 ; COc1cc(NS(=O)(=O)c2cccc(C#N)c2)c2[nH]ccc2c1 ; "NCS" ; COc1cc(NS(=O)(=O)c2cccc(C#N)c2)c2[nH]cc(Cl)c2c1]

### 1. Preparation of 3-cyano-N-(5-methoxy-1H-indol-7-yl)benzenesulfonamide

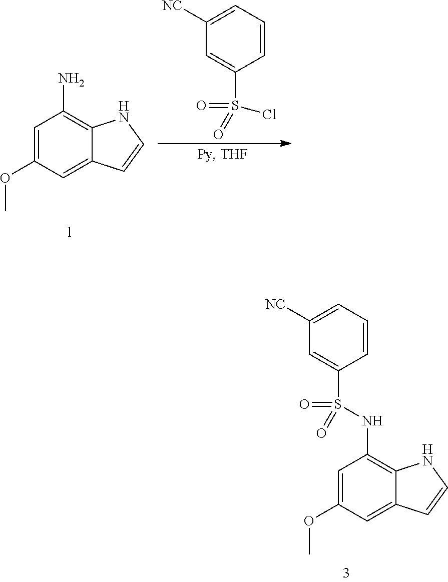 [SMILES: COc1cc(N)c2[nH]ccc2c1 ; N#Cc1cccc(S(=O)(=O)Cl)c1 ; COc1cc(NS(=O)(=O)c2cccc(C#N)c2)c2[nH]ccc2c1]

To a solution of 5-methoxy-1H-indol-7-amine (100.0 mg, 0.62 mmol) in THF (2 mL) were added pyridine (0.08 mL, 0.92 mmol) and 3-cyanobenzenesulfonyl chloride (124.3 mg, 0.62 mmol). The mixture was stirred at rt overnight. LCMS showed the desired mass was detected. The mixture was concentrated and purified by flash column eluting with PE/EA from 90/10 to 50/50 to give 3-cyano-N-(5-methoxy-1H-indol-7-yl)benzenesulfonamide (110.0 mg, 54.5%) as a white solid. LCMS (ESI) found: 328 [M+H]⁺.

### 2. Preparation of N-(3-chloro-5-methoxy-1H-indol-7-yl)-3-cyanobenzenesulfonamide

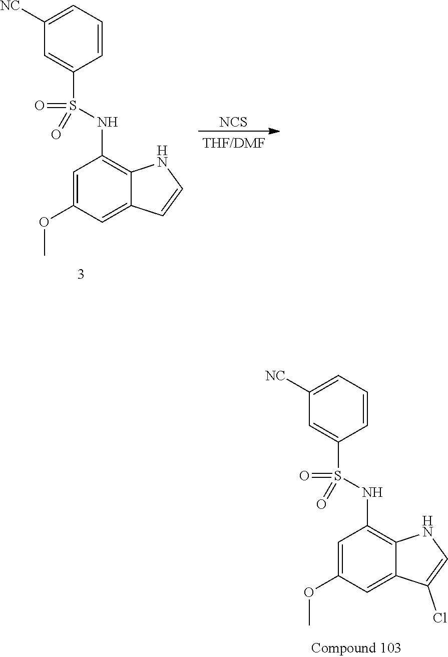 [SMILES: COc1cc(NS(=O)(=O)c2cccc(C#N)c2)c2[nH]ccc2c1 ; "NCS" ; COc1cc(NS(=O)(=O)c2cccc(C#N)c2)c2[nH]cc(Cl)c2c1]

To a solution of 3-cyano-N-(5-methoxy-1H-indol-7-yl)benzenesulfonamide (55.0 mg, 0.17 mmol) in THF/DMF (100/1, 2 mL) was added NCS (22.4 mg, 0.17 mmol). The mixture was stirred at rt for 5 h. LCMS showed the desired mass was detected. The mixture was quenched with aqueous Na₂S₂O₃ (10 mL) and extracted with DCM (15 mL×3). The combined organic layer was concentrated and purified by flash column eluting with PE/EA from 100/0 to 85/15 to give N-(3-chloro-5-methoxy-H-indol-7-yl)-3-cyanobenzenesulfonamide (Compound 103, 55.0 mg, 90.5%) as a white solid. LCMS (ESI) found: 362 [M+H]⁺. ¹H NMR (400 MHz, DMSO-d6) δ 10.89 (s, 1H), 10.23 (s, 1H), 8.18 (t, J=1.5 Hz, 1H), 8.14-8.06 (m, 1H), 8.03-7.94 (m, 1H), 7.74 (t, J=7.9 Hz, 1H), 7.43 (d, J=2.8 Hz, 1H), 6.74 (d, J=2.2 Hz, 1H), 6.41 (d, J=2.1 Hz, 1H), 3.70 (s, 3H).

The following compounds (Table 1) were prepared according to the similar procedure as described for Compound 56 from the corresponding amines.

TABLE 1

| Example | | | |
|---|---|---|---|
| # | Structure | Name | LCMS and ¹H NMR |
| Example 69 | 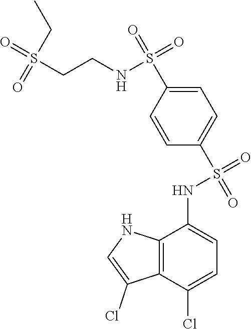 [SMILES: CCS(=O)(=O)CCNS(=O)(=O)c1ccc(S(=O)(=O)Nc2ccc(Cl)c3c(Cl)c[nH]c23)cc1] | N¹-(3,4-dichloro- 1H-indol-7-yl)- N⁴- (2- (ethylsulfonyl)ethyl) benzene-1,4- disulfonamide, Compound 3 | LC/MS ESI (m/z): 539.9 (M + H)⁺. ¹H NMR (400 MHz, MeOD) δ 7.98 − 7.93 (m, 2H), 7.88 − 7.83 (m, 2H), 7.30 (s, 1H), 6.86 (d, J = 8.1 Hz, 1H), 6.48 (d, J = 8.1 Hz, 1H), 3.30 − 3.27 (m, 2H), 3.25 − 3.21 (m, 2H), 3.11 (q, J = 7.4 Hz, 2H), 1.33 (t, J = 7.5 Hz, 3H). |
| Example 70 | 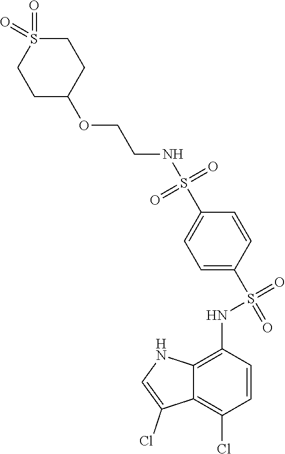 [SMILES: O=S1(=O)CCC(OCCNS(=O)(=O)c2ccc(S(=O)(=O)Nc3ccc(Cl)c4c(Cl)c[nH]c34)cc2)CC1] | N¹-(3,4-dichloro- 1H-indol-7-yl)-N4_ (2-((1,1- dioxidotetrahydro- 2H-thiopyran-4- yl)oxy)ethyl) benzene-1,4- disulfonamide, Compound 4 | LC/MS ESI (m/z): 595.9 (M + H)⁺. ¹H NMR (400 MHz, MeOD) δ 7.93 (d, J = 8.4 Hz, 2H), 7.85 (d, J = 8.5 Hz, 2H), 7.29 (s, 1H), 6.84 (d, J = 8.1 Hz, 1H), 6.53 (d, J = 8.1 Hz, 1H), 3.54 − 3.50 (m, 1H), 3.42 (t, J = 5.2 Hz, 2H), 3.23 − 3.18 (m, 2H), 3.07(t, J = 5.2 Hz, 2H), 2.85 (d, J = 13.5 Hz, 2H), 2.20 − 2.14 (m, 2H), 2.09 − 2.03 (m, 2H). |
| Example 71 | 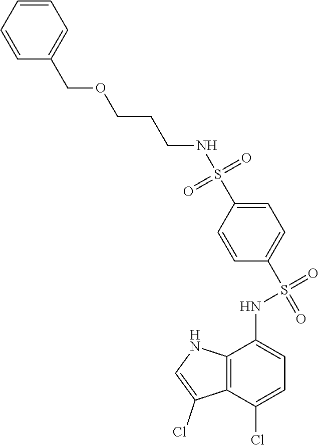 [SMILES: O=S(=O)(NCCCOCc1ccccc1)c1ccc(S(=O)(=O)Nc2ccc(Cl)c3c(Cl)c[nH]c23)cc1] | N¹-(3- (benzyloxy)propyl)- N⁴-(3,4-dichloro- 1H-indol-7- yl)benzene-1,4- disulfonamide, Compound 5 | LC/MS ESI (m/z): 566.2 (M − H)⁻. ¹H NMR (400 MHz, MeOD) δ 7.91 − 7.86 (m, 2H), 7.83 − 7.79 (m, 2H), 7.34 − 7.22 (m, 6H), 6.82 (d, J = 8.1 Hz, 1H), 6.48 (d, J = 8.1 Hz, 1H), 4.41 (s, 2H), 3.45 (t, J = 6.0 Hz, 2H), 2.95 (t, J = 6.9 Hz, 2H), 1.66 (dd, J = 12.8, 6.5 Hz, 2H). |
| Example 72 | 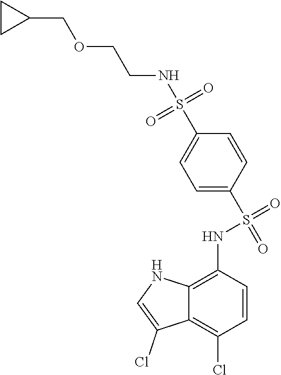 [SMILES: O=S(=O)(NCCOCC1CC1)c1ccc(S(=O)(=O)Nc2ccc(Cl)c3c(Cl)c[nH]c23)cc1] | N¹-(2- (cyclopropylmethoxy) ethyl)- N⁴-(3,4- dichloro-1H-indol- 7-yl)benzene-1,4- disulfonamide, Compound 6 | LC/MS ESI (m/z): 516.0 (M − H)⁻. ¹H NMR (400 MHz, DMSO) δ 11.37 (d, J = 2.2 Hz, 1H), 10.13 (s, 1H), 7.92 − 7.79 (m, 5H), 7.48 (d, J = 2.8 Hz, 1H), 6.86 (d, J = 8.2 Hz, 1H), 6.53 (d, J = 8.2 Hz, 1H), 3.23 − 3.20 (m, 2H), 3.00 (d, J = 6.8 Hz,2H), 2.85 (q, J = 5.8 Hz, 2H), 0.85 − 0.70 (m, 1H), 0.38 − 0.26 (m, 2H), 0.06 − 0.04 (m, 2H). |
| Example 73 | 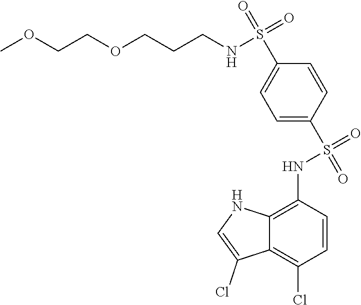 [SMILES: COCCOCCCNS(=O)(=O)c1ccc(S(=O)(=O)Nc2ccc(Cl)c3c(Cl)c[nH]c23)cc1] | N¹-(3,4-dichloro- 1H-indol-7-yl)- N⁴- (3-(2- methoxyethoxy) propyl)benzene-1,4- disulfonamide, Compound 7 | LC/MS ESI (m/z): 536.0 (M + H)⁺. ¹H NMR (400 MHz, MeOD) δ 7.92 (d, J = 8.5 Hz, 2H), 7.83 (d, J = 8.5 Hz, 2H), 7.30 (s, 1H), 6.85 (d, J = 8.1 Hz, 1H), 6.51 (d, J = 8.1 Hz, 1H), 3.48 (s, 4H), 3.42 (t, J = 5.9 Hz, 2H), 3.33 (s, 3H), 2.94 (t, J = 6.8 Hz, 2H), 1.67 − 1.59 (m, 2H). |
| Example 74 | 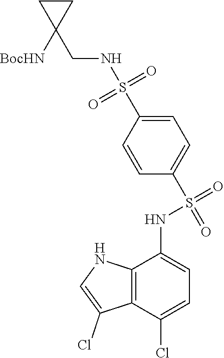 [SMILES: CC(C)(C)OC(=O)NC1(CNS(=O)(=O)c2ccc(S(=O)(=O)Nc3ccc(Cl)c4c(Cl)c[nH]c34)cc2)CC1] | tert-butyl (1-(((4- (N-(3,4-dichloro- 1H-indol-7- yl)sulfamoyl) phenyl)sulfonamido) methyl)cyclopropyl) carbamate, Compound 9 | LC/MS ESI (m/z): 589.2 (M + H)⁺. ¹H NMR (400 MHz, MeOD) δ 7.94 − 7.91 (m, 2H), 7.84 (d, J = 8.5 Hz, 2H), 7.32 (s, 1H), 6.86 (d, J = 8.1 Hz, 1H), 6.50 (d, J = 8.0 Hz, 1H), 2.98 (s, 2H), 1.42 (s, 9H), 0.68 − 0.65 (m, 4H). |
| Example 75 | 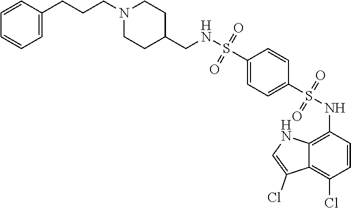 [SMILES: O=S(=O)(NCC1CCN(CCCc2ccccc2)CC1)c1ccc(S(=O)(=O)Nc2ccc(Cl)c3c(Cl)c[nH]c23)cc1] | N¹-(3,4-dichloro- 1H-indol-7-yl)- N⁴- ((1-(3- phenylpropyl) piperidin-4- yl)methyl)benzene- 1,4-disulfonamide, Compound 10 | LC/MS ESI (m/z): 635.1 (M + H)⁺. ¹H NMR (400 MHz, MeOD) δ 7.93 − 7.88 (m, 2H), 7.85 (d, J = 8.6 Hz, 2H), 7.28 (t, J = 7.4 Hz, 3H), 7.19 (dd, J = 13.8, 7.0 Hz, 3H), 6.83 (dd, J = 8.0, 4.8 Hz, 1H), 6.58 − 6.48 (m, 1H), 3.33 (m, 2H), 2.85(dd, J = 10.0, 6.5 Hz, 2H), 2.74 (d, J = 6.5 Hz, 2H), 2.70 − 2.56 (m, 4H), |
| | | | 2.03 − 1.92 (m, 2H), 1.85 |
| | | | (m, 2H), 1.64 (m, 1H), 1.35 |
| | | | (m, 2H). |
| Example 76 | 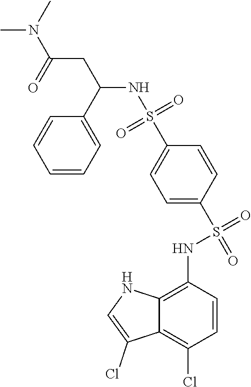 [SMILES: CN(C)C(=O)CC(NS(=O)(=O)c1ccc(S(=O)(=O)Nc2ccc(Cl)c3c(Cl)c[nH]c23)cc1)c1ccccc1] | 3-((4-(N-(3,4- dichloro-1H- indol-7-yl) sulfamoyl)phenyl) sulfonamido)- N,N-dimethyl-3- phenylpropanamide,, Compound 11 | LC/MS ESI (m/z): 595.0 (M + H)⁺. ¹H NMR (400 MHz, MeOD) δ 7.74 − 7.63 (m, 4H), 7.35 − 7.30 (m, 1H), 7.10 − 7.01 (m, 5H), 6.84 (d, J = 8.1 Hz, 1H), 6.50 (d, J = 8.1 Hz, 1H), 4.82 − 4.78 (m, 1H), 2.90 − 2.86 (s, 3H), 2.85 − 2.79 (m, 2H), 2.78 − 2.74 (s, 3H). |
| Example 77 | 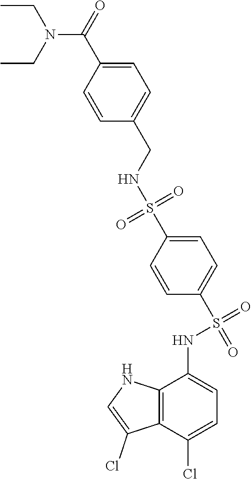 [SMILES: CCN(CC)C(=O)c1ccc(CNS(=O)(=O)c2ccc(S(=O)(=O)Nc3ccc(Cl)c4c(Cl)c[nH]c34)cc2)cc1] | 4-(((4-(N-(3,4- dichloro-1H-indol-7- yl)sulfamoyl)phenyl) sulfonamido) methyl)-N,N- diethylbenzamid, Compound 12 | LC/MS ESI (m/z): 609.0 (M + H)⁺. ¹H NMR (400 MHz, MeOD) δ 7.91 − 7.85 (m, 2H), 7.81 − 7.76 (m, 2H), 7.32 − 7.21 (m, 5H), 6.85 (d, J = 8.1 Hz, 1H), 6.48 (d, J = 8.1 Hz, 1H), 4.12 (s, 2H), 3.51 (dd, J = 13.5, 6.7 Hz, 2H), 3.20 (dd, J = 13.2, 6.5 Hz,2H), 1.22 (t, J = 6.6 Hz, 3H), 1.08 (t, J = 6.4 Hz, 3H). |
| Example 78 | 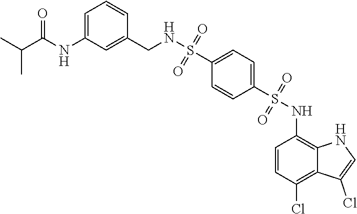 [SMILES: CC(C)C(=O)Nc1cccc(CNS(=O)(=O)c2ccc(S(=O)(=O)Nc3ccc(Cl)c4c(Cl)c[nH]c34)cc2)c1] | N-(3-(((4-(N-(3,4- dichloro-1H-indol- 7- yl)sulfamoyl)phenyl) sulfonamido) methyl)phenyl) isobutyramide, Compound 13 | LC/MS ESI (m/z): 594.9 (M + H)⁺. ¹H NMR (400 MHz, MeOD) δ 7.91 − 7.79 (m, 2H), 7.78 − 7.68 (m, 2H), 7.50 − 7.45 (m, 1H), 7.32 (dd, J = 7.2, 1.7 Hz, 1H), 7.29 (s, 1H), 7.12 (t, J = 7.9 Hz, 1H), 6.88 (d, J = 7.7 Hz, 1H), 6.84 (d, J = 8.1 Hz, 1H),6.46 (d, J = 8.1 Hz, 1H), 4.05 (s, 2H), 2.68 − |
| | | | 2.46 (m, 1H), 1.18 (d, J = |
| | | | 6.9 Hz, 6H) |
| Example 79 | 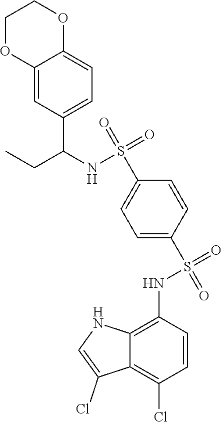 [SMILES: CCC(NS(=O)(=O)c1ccc(S(=O)(=O)Nc2ccc(Cl)c3c(Cl)c[nH]c23)cc1)c1ccc2c(c1)OCCO2] | N1-(3,4-dichloro- 1H-indol-7-yl)- N⁴- (1-(2,3- dihydrobenzo[b][1, 4]dioxin-6- yl)propyl)benzene- 1,4-disulfonamide, Compound 14 | LC/MS ESI (m/z): 594.2 (M − H)⁻. ¹H NMR (400 MHz, MeOD) δ 7.62 (d, J = 8.6 Hz, 2H), 7.57 (d, J = 8.5 Hz, 2H), 7.30 (s, 1H), 6.82 (d, J = 8.1 Hz, 1H), 6.52 (d, J = 8.2 Hz, 1H), 6.48 − 6.44 (m,1H), 6.36 (d, J = 8.0 Hz, 2H), 4.13 − 4.05 (m, 5H), 1.74 − 1.67 (m, 1H), 1.63 − 1.57 (m, 1H), 0.80 (t, J = 7.3 Hz, 3H). |
| Example 80 | 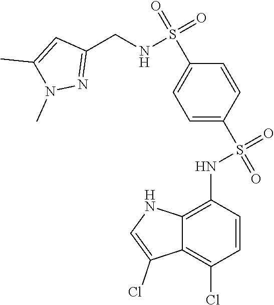 [SMILES: Cc1cc(CNS(=O)(=O)c2ccc(S(=O)(=O)Nc3ccc(Cl)c4c(Cl)c[nH]c34)cc2)nn1C] | N¹-(3,4-dichloro- 1H-indol-7-y1)- N⁴- ((1,5-dimethyl-1H- pyrazol-3- yl)methyl)benzene- 1,4-disulfonamide, Compound 15 | LC/MS ESI (m/z): 527.9 (M + H)⁺. ¹H NMR (400 MHz, MeOD) δ 7.85 − 7.79 (m, 2H), 7.77 − 7.71 (m, 2H), 7.30 (s, 1H), 6.84 (d, J = 8.1 Hz, 1H), 6.46 (d, J = 8.1 Hz, 1H), 5.71 (s, 1H), 4.00 (s, 2H), 3.55 (s, 3H), 2.09 (s, 3H). |
| Example 81 | 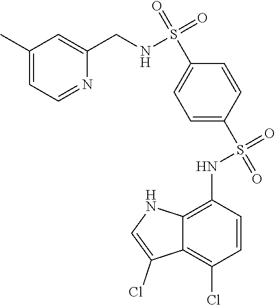 [SMILES: Cc1ccnc(CNS(=O)(=O)c2ccc(S(=O)(=O)Nc3ccc(Cl)c4c(Cl)c[nH]c34)cc2)c1] | N¹-(3,4-dichloro- 1H-indol-7-yl)- N⁴- ((4-methylpyridin- 2- yl)methyl)benzene- 1,4-disulfonamide, Compound 16 | LC/MS ESI (m/z): 524.9 (M + H)⁺. ¹H NMR (400 MHz, DMSO) δ 11.44 (s, 1H), 10.25 (s, 1H), 8.46 (t, J = 6.2 Hz, 1H), 8.12 (t, J = 4.4 Hz, 1H), 7.89 − 7.80 (m, 4H), 7.55 (s, 1H), 7.05 (s, 1H), 6.93 (d, J = 7.8 Hz, 2H), 6.62 (d, J = 8.2 Hz, 1H), 4.07 (d, J = 6.2 Hz, 2H), 2.19 (s, 3H). |
| Example 82 | 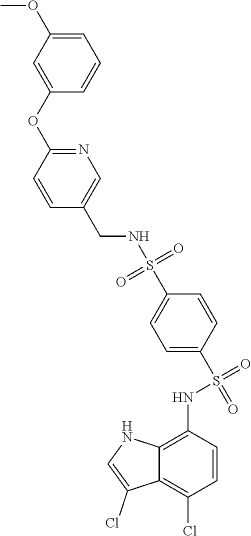 [SMILES: COc1cccc(Oc2ccc(CNS(=O)(=O)c3ccc(S(=O)(=O)Nc4ccc(Cl)c5c(Cl)c[nH]c45)cc3)cn2)c1] | N¹-(3,4-dichloro- 1H-indol-7-yl)- N⁴- ((6-(3- methoxyphenoxy) pyridin-3- yl)methyl)benzene- 1,4-disulfonamide, Compound 17 | LC/MS ESI (m/z): 633.3 (M + H)⁺. ¹H NMR (400 MHz, MeOD) δ 7.94 (d, J = 2.0 Hz, 1H), 7.87 (d, J = 8.7 Hz, 2H), 7.78 (d, J = 8.7 Hz, 2H), 7.63 (dd, J = 8.5, 2.4 Hz, 1H), 7.31 − 7.23 (m, 2H), 6.85 − 6.73 (m, 3H), 6.67 − 6.56 (m, 2H), 6.46 (d, J = 8.1Hz, 1H), 4.08 (s, 2H), 3.76 (s, 3H). |
| Example 83 | 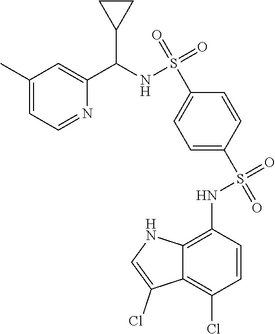 [SMILES: Cc1ccnc(C(NS(=O)(=O)c2ccc(S(=O)(=O)Nc3ccc(Cl)c4c(Cl)c[nH]c34)cc2)C2CC2)c1] | N¹-(cyclopropyl(4- methylpyridin-2- yl)methyl)- N⁴-(3,4- dichloro-1H-indol- 7-yl)benzene-1,4- disulfonamide, Compound 18 | LC/MS ESI (m/z): 565.0 (M + H)⁺. ¹H NMR (400 MHz, MeOD) δ 8.08 (d, J = 5.5 Hz, 1H), 7.76 − 7.70 (m, 2H), 7.69 − 7.65 (m, 2H), 7.32 (s, 1H), 7.03 (s, 1H), 6.96 (d, J =5.0 Hz, 1H), 6.84 (d, J = 8.1 Hz, 1H), 6.48 (d, J = 8.1 Hz, 1H), 3.62 (d, J = 9.0 Hz, 1H), 2.23 (s, 3H), 1.15 − 1.05 (m, 1H), 0.50 − 0.38 (m, 2H), 0.36 − 0.28 (m, 1H), 0.11 − 0.03 (m, 1H). |
| Example 84 | 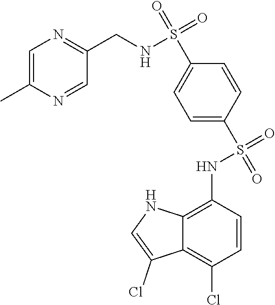 [SMILES: Cc1cnc(CNS(=O)(=O)c2ccc(S(=O)(=O)Nc3ccc(Cl)c4c(Cl)c[nH]c34)cc2)cn1] | N¹-(3,4-dichloro- 1H-indol-7-yl)-N4_ ((5-methylpyrazin-2- yl)methyl)benzene- 1,4-disulfonamide, Compound 19 | LC/MS ESI (m/z): 525.9 (M + H)⁺. ¹H NMR (400 MHz, MeOD) δ 8.37 − 8.35 (m, 1H), 8.27 − 8.25 (m, 1H), 7.87 − 7.84 (m, 2H), 7.77 − 7.74 (m, 2H), 7.30 (s, 1H), 6.84 (d, J = 8.1 Hz, 1H), 6.46 (d, J = 8.1 Hz, 1H), 4.24 (s, 2H), 2.46 (s, 3H). |
| Example 85 | 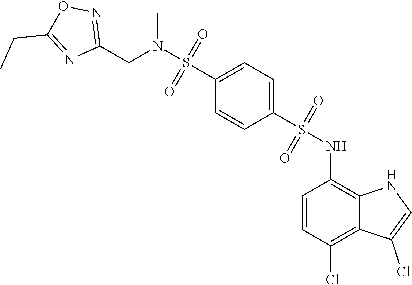 [SMILES: CCc1nc(CN(C)S(=O)(=O)c2ccc(S(=O)(=O)Nc3ccc(Cl)c4c(Cl)c[nH]c34)cc2)no1] | N¹-(3,4-dichloro- 1H-indol-7-yl)- N⁴- ((5-ethyl-1,2,4- oxadiazol-3- yl)methyl)-N4. methylbenzene-1,4- disulfonamide, Compound 20 | LC/MS ESI (m/z): 544.0 (M + H)⁺. ¹H NMR (400 MHz, MeOD) δ 7.92 − 7.86 (m, 2H), 7.85 − 7.79 (m, 2H), 7.31 (s, 1H), 6.86 (d, J = 8.1 Hz, 1H), 6.48 (d, J = 8.1 Hz, 1H),4.47 (s, 2H), 2.91 (s, 3H), 2.79 (q, J = 7.6 Hz, 2H), 1.25 (t, J = 7.6 Hz, 3H). |
| Example 86 | 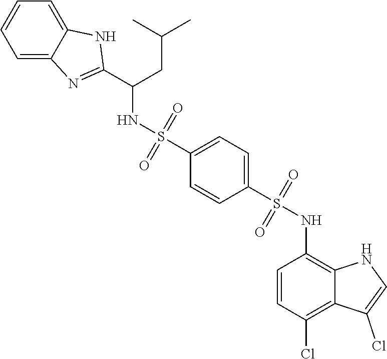 [SMILES: CC(C)CC(NS(=O)(=O)c1ccc(S(=O)(=O)Nc2ccc(Cl)c3c(Cl)c[nH]c23)cc1)c1nc2ccccc2[nH]1] | N¹-(1-(1H- benzo[d]imidazol- 2-yl)-3- methylbutyl)- N⁴- (3,4-dichloro-1H- indol-7-yl)benzene- 1,4-disulfonamide, Compound 21 | LC/MS ESI (m/z): 606.0 (M + H)⁺. ¹H NMR (400 MHz, MeOD) δ 7.77 − 7.72 (m, 2H), 7.59 − 7.50 (m, 2H), 7.42 − 7.34 (m, 2H), 7.30 (s, 1H), 7.19 − 7.13 (m, 2H), 6.76 (d, J = 8.1 Hz, 1H), 6.34 (d, J = 8.1 Hz, 1H), 4.59 (t, J = 7.8 Hz, 1H), 1.78 − 1.63(m, 2H), 1.50 − 1.35 (m, 1H), 0.87 (d, J = 6.6 Hz, 3H), 0.74 (d, J = 6.6 Hz, 3H). |
| Example 87 | 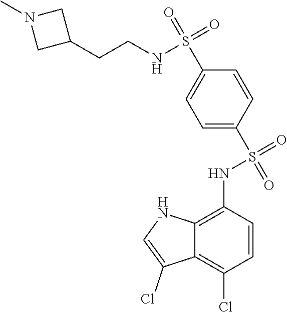 [SMILES: CN1CC(CCNS(=O)(=O)c2ccc(S(=O)(=O)Nc3ccc(Cl)c4c(Cl)c[nH]c34)cc2)C1] | N¹-(3,4-dichloro- 1H-indol-7-y1)- N⁴- (2-(1- methylazetidin-3- yl)ethyl)benzene- 1,4-disulfonamide, Compound 22 | LC/MS ESI (m/z): 517.3 (M + H)⁺. ¹H NMR (400 MHz, MeOD) 87.95 − 7.86 (m, 4H), 7.30 (s, 1H), 6.85 (d, J = 8.1 Hz, 1H), 6.62 − 6.51 (m, 1H), 3.45 − 3.37 (m, 2H), 3.21 − 3.13 (m, 1H), 2.99 − 2.86 (m, 3H), 2.63 (s, 3H), 2.45 − 2.32 (m, 1H), 2.07 − 1.97 (m, 1H), 1.59 − 1.47 (m, 1H). |
| Example 88 | 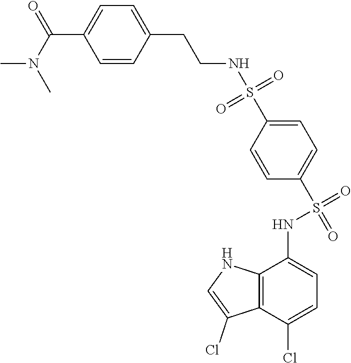 [SMILES: CN(C)C(=O)c1ccc(CCNS(=O)(=O)c2ccc(S(=O)(=O)Nc3ccc(Cl)c4c(Cl)c[nH]c34)cc2)cc1] | 4-(2-((4-(N-(3,4- dichloro-1H-indol- 7- yl)sulfamoyl)pheny 1)sulfonamido)ethyl)- N,N- dimethylbenzamide, Compound 23 | LC/MS ESI (m/z): 595.3 (M + H)⁺. ¹H NMR (400 MHz, DMSO) δ 11.46 (d, J = 2.5 Hz, 1H), 10.20 (s, 1H), 7.97 (t, J = 5.8 Hz, 1H), 7.93 − 7.87 (m, 4H), 7.53 (d, J = 2.8 Hz, 1H), 7.29 (d, J = 8.1 Hz, 2H), 7.19 (d, J = 8.1 Hz, 2H), 6.88 (d, J = 8.1 Hz,1H), 6.57 (d, J = 8.2 Hz, 1H), 3.03 − 2.98 (m, 2H), 2.93 (s, 6H), 2.67 (t, J = 7.3 Hz, 2H) |
| Example 89 | 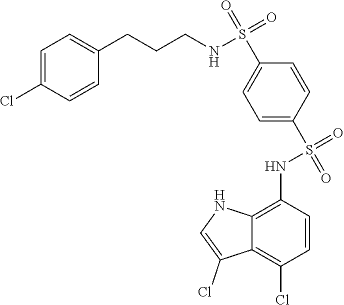 [SMILES: O=S(=O)(NCCCc1ccc(Cl)cc1)c1ccc(S(=O)(=O)Nc2ccc(Cl)c3c(Cl)c[nH]c23)cc1] | N¹-(3-(4- chlorophenyl)propyl)- N⁴-(3,4-dichloro- 1H-indol-7- yl)benzene-1,4- disulfonamide, Compound 24 | LC/MS ESI (m/z): 571.9 (M + H)⁺. ¹H NMR (400 MHz, DMSO) δ 11.44 (s, 1H), 10.22 (s, 1H), 7.93 − 7.86 (m, 5H), 7.53 (s, 1H), 7.29 (d, J = 8.4 Hz, 2H), 7.11 (d, J = 8.4 Hz, 2H), 6.88 (d, J = 8.2 Hz, 1H), 6.58 (d, J = 8.2 Hz, 1H), 2.73 (m, 2H), 2.47 (m, 2H), 1.65 − 1.54 (m, 2H). |
| Example 90 | 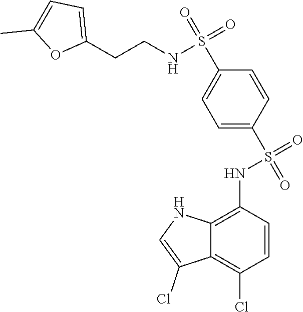 [SMILES: Cc1ccc(CCNS(=O)(=O)c2ccc(S(=O)(=O)Nc3ccc(Cl)c4c(Cl)c[nH]c34)cc2)o1] | N¹-(3,4-dichloro- 1H-indol-7-yl)-N4_ (2-(5-methylfuran- 2-yl)ethyl)benzene- 1,4-disulfonamide, Compound 25 | LC/MS ESI (m/z): 528.3 (M + H)⁺. ¹H NMR (400 MHz, MeOD) δ 7.91 − 7.86 (m, 2H), 7.84 − 7.77 (m, 2H), 7.28 (s, 1H), 6.78 (d, J = 8.1 Hz, 1H), 6.43 (d, J = 8.1 Hz, 1H), 5.88 − 5.77 (m, 2H), 3.11 (t, J = 7.2 Hz, 2H), 2.64 (t, J = 7.2 Hz, 2H), 2.16 (s, 3H). |
| Example 91 | 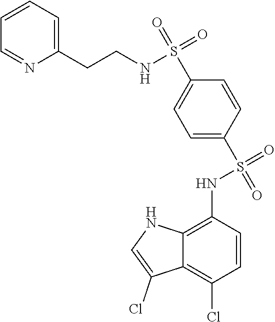 [SMILES: O=S(=O)(NCCc1ccccn1)c1ccc(S(=O)(=O)Nc2ccc(Cl)c3c(Cl)c[nH]c23)cc1] | N¹-(3,4-dichloro- 1H-indol-7-yl)- N⁴- (2-(pyridin-2- yl)ethyl)benzene- 1,4-disulfonamide, Compound 26 | LC/MS ESI (m/z): 524.9 (M + H)⁺. ¹H NMR (400 MHz, MeOD) δ 8.38 (d, J = 4.2 Hz, 1H), 7.89 − 7.85 (m, 2H), 7.81 − 7.77 (m, 2H), 7.69 (td, J = 7.7, 1.8 Hz, 1H), 7.28 (s, 1H), 7.25 − 7.17 (m, 2H), 6.78 (d, J = 8.1 Hz, 1H), 6.46 (d, J = 8.1 Hz, 1H), 3.24 (t, J = 7.1 Hz, 2H), 2.86 (t, J = 7.1 Hz, 2H). |
| Example 92 | 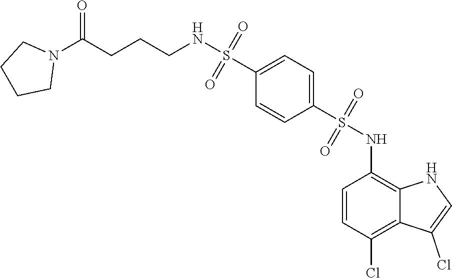 [SMILES: O=C(CCCNS(=O)(=O)c1ccc(S(=O)(=O)Nc2ccc(Cl)c3c(Cl)c[nH]c23)cc1)N1CCCC1] | N¹-(3,4-dichloro- 1H-indol-7-yl)- N⁴- (4-oxo-4- (pyrrolidin-1- yl)butyl)benzene- 1,4-disulfonamide, Compound 27 | LC/MS ESI (m/z): 559.0 (M + H)⁺. ¹H NMR (400 MHz, MeOD) δ 7.93 − 7.90 (m, 2H), 7.85 − 7.81 (m, 2H), 7.30 (s, 1H), 6.84 (d, J = 8.1 Hz, 1H), 6.48 (d, J = 8.1 Hz, 1H), 3.43 − 3.34 (m, 4H), 2.90 (t, J = 6.7 Hz, 2H), 2.29 (t, J = 7.1 Hz, 2H), 1.98 −1.91 (m, 2H), 1.89 − 1.82 (m, 2H), 1.73 − 1.67 (m, 2H). |
| Example 93 | 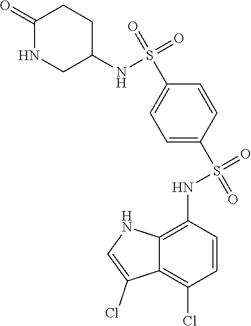 [SMILES: O=C1CCC(NS(=O)(=O)c2ccc(S(=O)(=O)Nc3ccc(Cl)c4c(Cl)c[nH]c34)cc2)CN1] | N¹-(3,4-dichloro- 1H-indol-7-yl)- N⁴- (6-oxopiperidin-3- yl)benzene-1,4- disulfonamide, Compound 31 | LC/MS ESI (m/z): 517.3 (M + H)⁺. ¹H NMR (400 MHz, MeOD-d4) δ 7.96 − 7.90 (m, 2H), 7.87 − 7.81 (m, 2H), 7.30 (s, 1H), 6.85 (d, J = 8.0, 1H), 6.50 (d, J =8.0, 1H), 3.75 − 3.68 (m, 1H), 2.97 − 2.87 (m, 2H), 2.35 − 2.15 (m, 3H), 1.88 − 1.80 (m, 1H). |
| Example 94 | 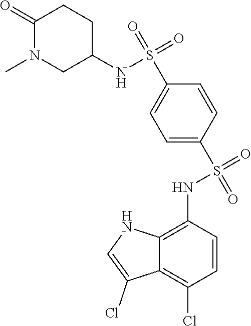 [SMILES: CN1CC(NS(=O)(=O)c2ccc(S(=O)(=O)Nc3ccc(Cl)c4c(Cl)c[nH]c34)cc2)CCC1=O] | N¹-(3,4-dichloro- 1H-indol-7-yl)-N⁴- (1-methyl-6- oxopiperidin-3- yl)benzene-1,4- disulfonamide, Compound 32 | LC/MS ESI (m/z): 531.0 (M + H)⁺. ¹H NMR (400 MHz, MeOD) δ 7.99 − 7.95 (m, 2H), 7.87 − 7.83 (m, 2H), 7.31 (s, 1H), 6.84 (d, J = 8.1 Hz, 1H), 6.50 (d, J = 8.1 Hz, 1H), 3.62 − 3.56 (m, 1H), 3.43 − 3.38 (m, 1H), 3.13 − 3.08 (m, 1H), 2.81 (s, 3H), 2.42 − 2.26 (m, 2H), 1.75 − 1.66 (m, 2H). |
| Example 95 | 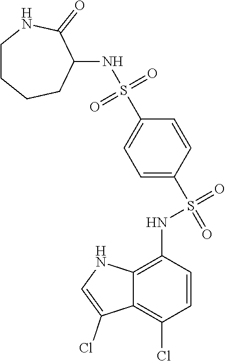 [SMILES: O=C1NCCCCC1NS(=O)(=O)c1ccc(S(=O)(=O)Nc2ccc(Cl)c3c(Cl)c[nH]c23)cc1] | N¹-(3,4-dichloro- 1H-indol-7-yl)- N⁴- (2-oxoazepan-3- yl)benzene-1,4- disulfonamide, Compound 33 | LC/MS ESI (m/z): 530.9 (M + H)⁺. ¹H NMR (400 MHz, MeOD) δ 7.96 − 7.91 (m, 2H), 7.84 − 7.79 (m, 2H), 7.28 (s, 1H), 6.82 (d, J = 8.0 Hz, 1H), 6.46 (d, J = 8.0 Hz, 1H), 4.02 − 3.93 (m, 1H), 3.16 − 3.04 (m, 2H), 1.93 − 1.47 (m, 6H). |
| Example 96 | 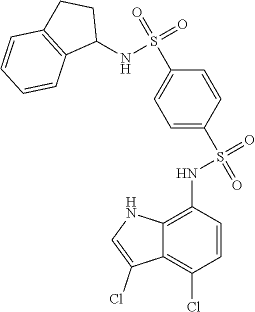 [SMILES: O=S(=O)(Nc1ccc(Cl)c2c(Cl)c[nH]c12)c1ccc(S(=O)(=O)NC2CCc3ccccc32)cc1] | N¹-(3,4-dichloro- 1H-indol-7-yl)- N⁴- (2,3-dihydro-1H- inden-1- yl)benzene-1,4- disulfonamide, Compound 34 | LC/MS ESI (m/z): 533.9 (M − H)⁻. ¹H NMR (400 MHz, DMSO) δ 11.48 (d, J = 2.4 Hz, 1H), 10.24 (s, 1H), 8.36 (d, J = 8.8 Hz, 1H), 8.04 − 7.99 (m, 2H),7.96 − 7.90 (m, 2H), 7.56 (d, J = 2.8 Hz, 1H), 7.23 − 7.17 (m, 2H), 7.10 − 7.05 (m, 1H), 7.01 − 6.97 (m, 1H), 6.94 − 6.90(m, 1H), 6.66 − 6.62 (m, 1H), 4.69 (q, J = 7.9 Hz, 1H), 2.84 − 2.76 (m, 1H), 2.69 − 2.61 (m, 1H), 1.91 − 1.83 (m, 1H),1.49 − 1.39 (m, 1H). |
| Example 97 | 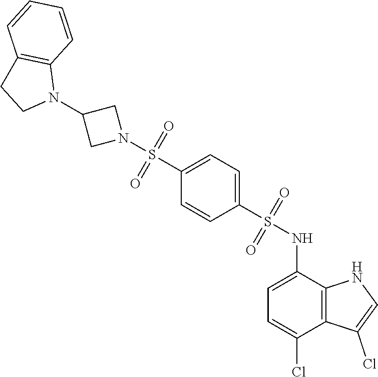 [SMILES: O=S(=O)(Nc1ccc(Cl)c2c(Cl)c[nH]c12)c1ccc(S(=O)(=O)N2CC(N3CCc4ccccc43)C2)cc1] | N-(3,4-dichloro- 1H-indol-7-yl)-4- ((3-(indolin-1- yl)azetidin-1- yl)sulfonyl) benzene sulfonamide, Compound 35 | LC/MS ESI (m/z): 577.0 (M + H)⁺. ¹H NMR (400 MHz, DMSO) δ 11.58 (s, 1H), 10.69 − 10.08 (m, 1H), 8.02 (s, 4H), 7.55 (s, 1H), 7.01 − 6.90 (m, 2H), 6.83 (d, J = 8.2 Hz, 1H), 6.66 − 6.56 (m, 2H), 6.35 (d, J = 7.8 Hz, 1H), 4.29 − 4.22 (m, 1H), 4.01 (t, J = 8.2 Hz, 2H), 3.76 (t, J = 8.6, 6.6 Hz, 2H), 2.85 (t, J = 8.2 Hz, 2H), 2.62 (t, J = 8.1 Hz, 2H). |
| Example 98 | 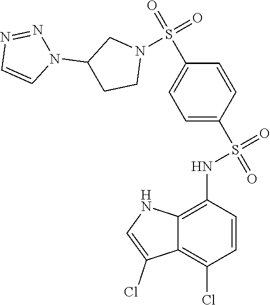 [SMILES: O=S(=O)(Nc1ccc(Cl)c2c(Cl)c[nH]c12)c1ccc(S(=O)(=O)N2CCC(n3ccnn3)C2)cc1] | 4-((3-(1H-1,2,3- triazol-1- yl)pyrrolidin-1- yl)sulfonyl)-N-(3,4- dichloro-1H-indol-7- yl)benzenesulfona mide, Compound 36 | LC/MS ESI (m/z): 540.9 (M + H)⁺. ¹H NMR (400 MHz, MeOD) δ 7.97 − 7.80 (m, 5H), 7.65 (d, J = 7.1 Hz, 1H), 7.29 (d, J = 6.9 Hz, 1H), 6.83 (d, J = 7.6 Hz, 1H), 6.55 (t, J = 7.4 Hz, 1H), 5.20 (s, 1H), 3.76 (d, J = 22.6 Hz, 2H), 3.57 − 3.48 (m, 2H),2.42 (d, J = 5.7 Hz, 2H). |
| Example 99 | 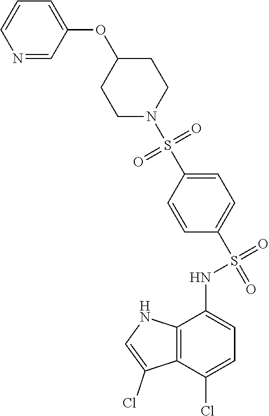 [SMILES: O=S(=O)(Nc1ccc(Cl)c2c(Cl)c[nH]c12)c1ccc(S(=O)(=O)N2CCC(Oc3cccnc3)CC2)cc1] | N-(3,4-dichloro- 1H-indol-7-yl)-4- ((4-(pyridin-3- yloxy)piperidin-1- yl)sulfonyl)benzene sulfonamide, Compound 39 | LC/MS ESI (m/z): 581.3 (M + H)⁺. ¹H NMR (400 MHz, MeOD) δ 8.18 (d, J = 2.6 Hz, 1H), 8.11 (dd, J = 4.5, 0.8 Hz, 1H), 7.91 − 7.86 (m, 4H), 7.42 − 7.38 (m, 1H), 7.35 − 7.31 (m, 2H), 6.84 (d, J = 8.1 Hz, 1H), 6.47 (d, J = 8.1 Hz, 1H), 4.54 (ddd, J = 7.4, 5.3, 2.4 Hz, 1H), 3.29 − 3.25 (m, 2H), 3.09 − 3.02 (m, 2H), 2.07 − 2.00 (m, 2H), 1.87 − 1.80 (m, 2H). |
| Example 100 | 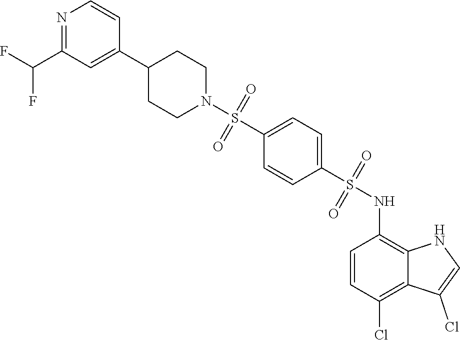 [SMILES: O=S(=O)(Nc1ccc(Cl)c2c(Cl)c[nH]c12)c1ccc(S(=O)(=O)N2CCC(c3ccnc(C(F)F)c3)CC2)cc1] | N-(3,4-dichloro- 1H-indol-7-yl)-4- ((4-(2- (difluoromethyl) pyridin-4-yl) piperidin-1- yl)sulfonyl)benzene sulfonamide, Compound 40 | LC/MS ESI (m/z): 614.9 (M + H)⁺. 1 H NMR (400 MHz, CDCl 3) δ 9.26 (s, 1H), 8.57 (d, J = 5.1 Hz, 1H), 7.87 − 7.77 (m, 4H), 7.44 (s, 1H), 7.32 (d, J = 2.6 Hz, 1H), 7.20 (d, J = 5.2 Hz, 1H), 6.87 (d, J = 8.1 Hz, 1H), 6.78 − 6.44 (m, 2H), 6.28 (d, J = 8.0 Hz, 1H), 3.97 (d, J = 11.7 Hz, 2H), 2.55 (tt, J = 11.8, 3.4 Hz, 1H), 2.35 (td, J = 11.8, 2.3 Hz, 2H), 2.00 − 1.91 (m, 2H), 1.87 (td, J = 12.0, 3.6 Hz, 2H). |
| Example 101 | 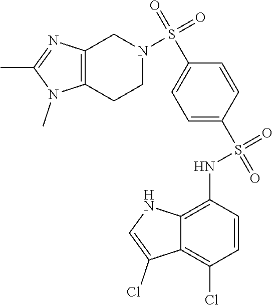 [SMILES: Cc1nc2c(n1C)CCN(S(=O)(=O)c1ccc(S(=O)(=O)Nc3ccc(Cl)c4c(Cl)c[nH]c34)cc1)C2] | N-(3,4-dichloro- 1H-indol-7-yl)-4- ((1,2-dimethyl- 1,4,6,7-tetrahydro- 5H-imidazo[4,5- c]pyridin-5- yl)sulfonyl)benzene sulfonamide, Compound 53 | LC/MS ESI (m/z): 554.2 (M + H)⁺. ¹H NMR (400 MHz, MeOD) 7.90 (d, J = 8.6 Hz, 2H), 7.83 (d, J = 8.6 Hz, 2H), 7.20 (s, 1H), 6.82 (d, J = 8.1 Hz, 1H), 6.53 (d, J = 8.1 Hz, 1H), 4.09 (s, 2H), 3.44 (s, 3H), 3.41 (t, J = 5.8 Hz, 2H), 2.64 (t, J = 5.6 Hz, 2H), 2.40 (s, 3H). |
| Example 102 | 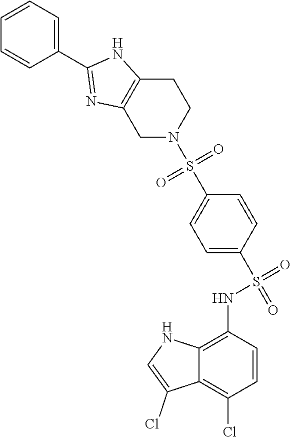 [SMILES: O=S(=O)(Nc1ccc(Cl)c2c(Cl)c[nH]c12)c1ccc(S(=O)(=O)N2CCc3[nH]c(-c4ccccc4)nc3C2)cc1] | N-(3,4-dichloro- 1H-indol-7-yl)-4- ((2-phenyl-1,4,6,7- tetrahydro-5H- imidazo[4,5- c]pyridin-5- yl)sulfonyl) benzene sulfonamide, Compound 54 | LC/MS ESI (m/z): 602.3 (M + H)⁺. ¹H NMR (400 MHz, MeOD) δ 7.93 (d, J = 8.6 Hz, 2H), 7.84 − 7.78 (m, 4H), 7.43 (t, J = 7.4 Hz, 2H), 7.36 (t, J = 7.3 Hz, 1H), 7.18 (s, 1H), 6.79 (d, J = 8.1 Hz, 1H),6.46 (d, J = 8.1 Hz, 1H), 4.21 (s, 2H), 3.44 (t, J = 5.7 Hz, 2H), 2.75 (t, J = 5.6 Hz, 2H). |
| Example 103 | 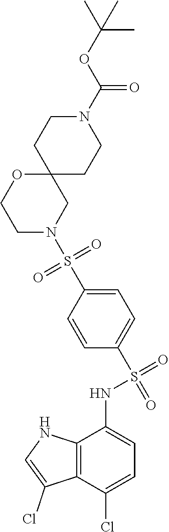 [SMILES: CC(C)(C)OC(=O)N1CCC2(CC1)CN(S(=O)(=O)c1ccc(S(=O)(=O)Nc3ccc(Cl)c4c(Cl)c[nH]c34)cc1)CCO2] | tert-butyl 4-((4-(N- (3,4-dichloro-1H- indol-7- yl)sulfamoyl) phenyl) sulfonyl)-1-oxa- 4,9- diazaspiro[5.5] undecane-9- carboxylate, Compound 55 | LC/MS ESI (m/z): 657.3 (M − H)⁻. ¹H NMR (400 MHz, DMSO) δ 11.43 (d, J = 2.5 Hz, 1H), 10.23 (d, J = 11.0 Hz, 1H), 7.91 − 7.85 (m, 4H), 7.53 (t, J = 2.8 Hz, 1H), 6.95 (d, J = 8.1 Hz, 1H), 6.60 − 6.57 (m, 1H), 3.71 (dd, J = 9.6, 4.7 Hz, 2H), 3.58 (d, J = 13.0 Hz, 2H), 3.00 (s, 2H), 2.85 (dd, J = 5.1, 3.8 Hz, 2H), 2.71 (s, 2H), 2.00 (d, J = 7.4 Hz, 1H), 1.74 (d, J = 13.3 Hz, 2H), 1.44 (s, 1H), 1.39 (s, 9H). |
| Example 104 | 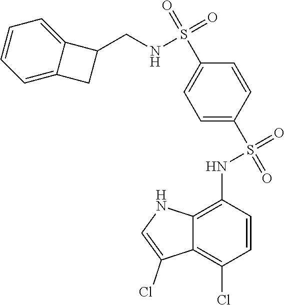 [SMILES: O=S(=O)(NCC1Cc2ccccc21)c1ccc(S(=O)(=O)Nc2ccc(Cl)c3c(Cl)c[nH]c23)cc1] | N¹- (bicyclo[4.2.0] octa-1,3,5-trien-7- ylmethyl)- N⁴-(3,4- dichloro-1H-indol- 7-yl)benzene-1,4- disulfonamide | LC/MS ESI (m/z): 534.0 (M − H)⁻. 1 H NMR (400 MHz, MeOD) δ 7.95 − 7.92 (m, 2H), 7.83 − 7.80 (m, 2H), 7.25 (s, 1H), 7.15 (dd, J = 14.8, 7.4 Hz, 2H), 7.03 (d, J = 7.9 Hz, 2H), 6.70 (d, J = 8.1 Hz, 1H), 6.41 (d, J = 8.1 Hz, 1H), 3.50 − 3.44 (m, 1H),3.25 − 3.16 (m, 2H), 3.07 (dd, J = 13.0, 8.1 Hz, 1H), 2.75 (dd, J = 14.3, 2.3 Hz, 1H). |

### Example 105: Quantitative Evaluation of Compound-Induced Ternary Complex Formation Using Amplified Luminescent Proximity Homogeneous Assay

Compound-induced DCAF15-RBM39 ternary complex formation was monitored using AlphaLISA® assays (Perkin Elmer, Waltham, MA). Test compounds were first collected in a 384-well microplate (cat. #781280, Grenier Bio-One, Monroe, NC) as 2 mM stocks in 100% dimethyl sulfoxide (DMSO). These stocks were then serially diluted in DMSO by 3-fold into a new plate (cat. #781280, Grenier Bio-One, Monroe, NC) using a Mosquito HTS dispenser (mosquito@ HTS, SPT Labtech, Boston, MA). 0.2 ul of these diluted stocks were then dispensed into a 384-well Optiplate (cat. #6007290, Perkin Elmer, Waltham, MA) in duplicate wells, to assemble the final assay plate consisting of duplicated dose ranges. Separately, a protein mixture consisting of His-tagged DCAF15 complex and biotin-carrying RBM39, each at 408 nM (2.04×), was prepared in 50 mM HEPES, pH 7.5, 100 mM NaCl, 2 mM MgCl2, 0.1% BSA, 2 mM TCEP, 0.05% Tween-20 (dilution buffer). 9.8 ul of this protein mixture was then mixed with the compounds in the assay plate and incubated for one hour at room temperature. Reaction mixtures with no compounds added were included as negative controls, while a His-tagged and biotinylated GST protein was included to track general assay performance. Nickel Chelate AlphaLISA Acceptor beads (cat. #AL108M, Perkin Elmer, MA) and AlphaScreen Streptavidin Donor beads (cat. #6760002, Perkin Elmer, Waltham, MA) were prepared separately in the dilution buffer as 4× stocks (80 ug/ml). The beads were then added to the assay plate sequentially with incubation at room temperature for 1-2 hours after each addition. Signals were recorded using EnVision 2104 Multilabel Plate Reader (Perkin Elmer, Waltham, MA) and titration curves were generated in GraphPad Prism (7.00, GraphPad, La Jolla, CA). Results are summarized in Table 2.

### Example 106: Measurement of Binary Interaction Between Compounds and DCAF15 Using Homogeneous Time Resolved Fluorescence (HTRF)

DCAF15 interactions with test compounds were monitored using HTRF competition assays (Cisbio, Bedford, MA). Test compounds were first collected in a 384-well microplate (cat. #781280, Grenier Bio-One, Monroe, NC) as 10 mM stocks in 100% dimethyl sulfoxide (DMSO). A second stock plate (cat. #781280, Grenier Bio-One, Monroe, NC) was prepared through 3-fold serial dilution in DMSO of the afore-mentioned stocks using a Mosquito HTS dispenser (Mosquito® HTS, SPT Labtech, Boston, MA). 0.2 ul of these diluted stocks were then dispensed into a 384-well Optiplate (cat. #6007290, Perkin Elmer, Waltham, MA) in duplicate wells, to assemble the assay plate. The tracer molecule containing an Alexa Fluor 647 probe was prepared in 25 mM HEPES pH 7.5, 100 mM NaCl, 0.1 mg/ml BSA, 0.005% Tween 20, 0.5 mM TCEP (dilution buffer) to 612 nM (2.04×). Separately, His-tagged DCAF15 complex was prepared at 32 nM (4×) in the dilution buffer, and mAb Anti-6His-Eu cryptate Gold (cat. #61H12KLA, Cisbio, Bedford, MA) at 4× dilution in the detection buffer (cat. #61DB9RDF, Cisbio, Bedford, MA). These two solutions were mixed and incubated for 15 min at room temperature. 9.8 ul of the tracer solution and 10 ul of the DCAF15/mAb Anti-6His-Eu cryptate Gold mixture were added sequentially to each well in the assay plate. Reaction mixtures with no DCAF15 added were included as positive controls. The final mixture was incubated for an hour at room temperature and spun down briefly before data collection at 615 nm and 666 nm using EnVision 2104 Multilabel Plate Reader (Perkin Elmer, Bedford, MA). Binding was estimated based on ratiometric analysis and titration curves were generated in GraphPad Prism (7.00, GraphPad, La Jolla, CA). Results are summarized in Table 2.

### Example 107: Western Blot Assay Procedure for Assessing Degradation of RBM39 in HCT116 Cells

HCT116 cells were used with passage numbers ranging from 13 to 25. Cells were seeded in 12-well plated at a density of 1 million cells per well in 1 mL cell culture medium containing McCoy's 5A medium (Cat. #16600082, ThermoFisher, Waltham, MA), 10% Fetal Bovine Serum (Cat. #26140, ThermoFisher, Waltham, MA) and 1% Pen-Strep (Cat. #. 15070063, ThermoFisher, Waltham, MA) and incubated overnight. The next morning, compounds were serially diluted (1:3, 9 points) in the medium separately and were prepared 2× the final assay concentration. Subsequently, 1.0 mL medium containing DMSO or the appropriate concentration of each compound was added to respective wells. The final DMSO concentration in each well was 0.1%. After 24 hour incubation with compounds, cell were harvested (approximately 90-100% confluency at the time of harvest). Cells were then washed once with 1 mL of ice cold Dulbecco's phosphate-buffered saline (Cat. #28374, ThermoFisher, Waltham, MA) and then lysed with 100 μl of lysis buffer containing 1× RIPA (Cat. #. 89900, ThermoFisher, Waltham, MA) and HALT protease and phosphatase inhibitor (Cat. #78442, ThermoFisher, Waltham, MA) on ice for 10 minutes with intermittent rocking. Lysates were then sonicated in a sonication bath (3 pulses of 10 sec each with 1 min incubation on ice between each pulse). Samples were then centrifuged for 20 min at 14,000 rpm for 20 min. Supernatants were then transferred to fresh microfuge tubes and stored frozen at −80° C. until use. Total protein quantitation in lysate samples was done using the BCA protein quantitation kit following the manufacturer's instructions. For polyacrylamide gel electrophoresis, each sample (15-30 uL total protein), was diluted with Laemmli sample buffer (Cat. #. 1610747, BioRad, Hercules, CA) by adding 9 ul of lysate to 3 ul of sample buffer containing 10% β-mercaptoethanol. Samples were then denatured at 95° C. for 5 minutes in a heat block, centrifuged at 14,000 rpm for 5 min, and placed on ice. Subsequently, 12 ul of each sample was loaded on 4-20% Tris/Glycine gels and electrophoresed in 1×Tris/Glycine SDS buffer at 80 (constant) volts for 2 h. Resolved samples were then transferred from gels to PVDF membrane using the BioRad Turbo-transfer unit with the Turbo default program (Cat. #. 1704150EDU, BioRad, Hercules, CA). For immunoblotting, membranes were first blocked for 1 hour at room temperature using Odyssey Blocking Buffer (Cat. #: 927-60001, Li-cor, Lincoln, NE) with continuous rocking. Membrane was then incubated with primary antibodies, anti-RBM39 (Sigma Cat. #: HPA001591, St. Louis, MO) and anti-β-actin (Cell Signaling Cat. #3700S, Danvers, MA), diluted 1000 fold each, at 4° C. for overnight. Subsequently, membrane was washed three times for at room temperature with continuous rocking (5 minutes each wash) using TBS buffer containing 0.1% Tween-20. After the final wash, secondary antibodies, IRDye 680 RD Goat anti Mouse IgG H+L (Cat. #: 926-68070, Li-cor, Lincoln, NE), and IRDye 800CW Goat anti Rabbit IgG H+L (Cat. #: 926-32211, Li-cor, Lincoln, NE), each diluted 10,000-fold, were added to the membranes and incubated at room temperature for 1 h with continuous rocking. Membranes were then washed 3 times with 1×TBS and scanned using Odessey CLx imaging system (Li-cor, Lincoln, NE) using default settings. Band intensity in images was quantified using the Image Studio Lite software (Version 5.2). Signal intensity for RBM-39 specific bands were normalized using the signal intensity for β-actin-specific band, and percent degradation was assessed by comparing RBM39 signal in compound-treated vs. DMSO-treated samples. Results are summarized in Table 2.

### Example 108: Measurement of HCT116 Cell Viability Using CellTiter-Glo

HCT116 cells with the passage number ranging from 13 to 25 were plated in an opaque-walled 384 well plate (Corning, Cat. #3765, Corning, NY) at a density of 1000 cells/well in 50 μl medium containing McCoy's 5A medium (Cat. #16600082, ThermoFisher, Waltham, MA), 10% Fetal Bovine Serum (Cat. #26140, ThermoFisher, Waltham, MA) and 1% Pen-Strep (Cat. #. 15070063, ThermoFisher, Waltham, MA). After overnight incubation, cells were treated with various concentrations of compounds (0-10 μM) in DMSO (final concentration of 0.2% DMSO) for 72 h. CellTiter-Glo® Reagent (Cat. #G7573, Promega, Waltham, MA) was then added to all wells. The plates were placed on an orbital shaker to mix the content and induce cell lysis. The plates were then incubated at room temperature for 10 minutes to stabilize luminescent signal. The luminescence was recorded using Envision multilabel plate reader (Perkin-Elmers, Waltham, MA) and the data was fit by using Excel-fit (Microsoft, Redmond, WA). Results are summarized in Table 2.

TABLE 2
Biological Data

| | Ternary- | | | |
|---|---|---|---|---|
| | RBM39 | | | 3-Day CTG |
| | (RRM2) | DCAF15 | DC₅₀ HCT116 | IC₅₀ in |
| | EC₅₀ (nM) | IC₅₀ | 4 h (nM) | HCT116 (nM) |
| | + <20 uM | + <100 uM | + <5,000 nM | + <5,000 nM |
| Cmpd. | ++ - <5 uM | ++ <20 uM | ++ <1,000 nM | ++ <1,000 nM |
| # | +++ <1 uM | +++ <5 uM | +++ <100 nM | +++ <100 nM |
| Indisulam | +++ | | | ++ |
| E-7820 | +++ | ++ | +++ | ++ |
| Tasisulam | ++ | | | |
| CQS | ++ | | +++ | + |
| 1 | +++ | + | ++ | ++ |
| 2 | +++ | ++ | | ++ |
| 3 | +++ | ++ | | |
| 4 | +++ | +++ | | |
| 5 | + | + | | |
| 6 | ++ | +++ | | |
| 7 | ++ | ++ | | |
| 8 | +++ | +++ | ++ | |
| 9 | + | ++ | | |
| 10 | +++ | +++ | | |
| 11 | + | +++ | | |
| 12 | + | | | |
| 13 | + | ++ | | |
| 14 | + | ++ | | |
| 15 | + | +++ | | |
| 16 | + | +++ | | |
| 17 | + | ++ | | |
| 18 | ++ | ++ | | |
| 19 | +++ | +++ | | |
| 20 | ++ | +++ | | |
| 22 | +++ | +++ | | |
| 23 | ++ | ++ | | |
| 25 | + | ++ | | |
| 26 | ++ | +++ | | |
| 27 | +++ | +++ | | |
| 28 | +++ | + | | |
| 29 | +++ | +++ | | |
| 30 | +++ | +++ | | |
| 31 | +++ | +++ | | |
| 32 | +++ | +++ | | |
| 33 | +++ | +++ | | |
| 34 | + | | | |
| 36 | ++ | +++ | | |
| 37 | +++ | | | |
| 38 | +++ | +++ | | |
| 39 | ++ | ++ | | |
| 41 | +++ | +++ | | |
| 42 | +++ | +++ | ++ | +++ |
| 43 | +++ | +++ | | |
| 44 | +++ | +++ | | |
| 45 | +++ | +++ | | |
| 46 | +++ | +++ | | |
| 47 | +++ | +++ | | |
| 48 | +++ | ++ | | |
| 49 | ++ | | | |
| 50 | +++ | | | |
| 51 | +++ | ++ | | |
| 52 | +++ | +++ | | |
| 53 | ++ | +++ | | |
| 54 | ++ | + | | |
| 55 | ++ | ++ | | |
| 57 | +++ | +++ | | |
| 58 | +++ | +++ | | |
| 59 | +++ | +++ | | |
| 60 | +++ | +++ | | |
| 61 | ++ | +++ | | |
| 62 | +++ | +++ | | |
| 63 | +++ | +++ | | |
| 64 | ++ | + | | |
| 65 | ++ | +++ | | |
| 66 | +++ | +++ | | |
| 67 | +++ | +++ | | |
| 68 | +++ | +++ | | |
| 69 | +++ | + | | |
| 70 | +++ | +++ | | |
| 71 | +++ | +++ | | |
| 72 | +++ | ++ | | |
| 73 | | + | | |
| 74 | +++ | ++ | +++ | |
| 75 | ++ | +++ | | |
| 76 | ++ | ++ | | |
| 77 | +++ | +++ | | |
| 78 | +++ | +++ | | |
| 79 | +++ | +++ | | |
| 80 | +++ | ++ | | |
| 81 | +++ | +++ | | |
| 82 | +++ | ++ | | |
| 83 | +++ | ++ | | |
| 84 | +++ | ++ | | |
| 85 | +++ | ++ | | |
| 86 | +++ | ++ | | |
| 87 | ++ | ++ | | |
| 88 | +++ | ++ | | |
| 89 | +++ | +++ | | |
| 90 | +++ | +++ | | |
| 91 | +++ | + | | |
| 92 | +++ | ++ | | |
| 93 | +++ | | | |
| 94 | +++ | ++ | | |
| 95 | +++ | ++ | +++ | |
| 96 | +++ | ++ | +++ | |
| 97 | +++ | ++ | | |
| 98 | +++ | | | |
| 100 | ++ | | | ++ |
| 101 | ++ | + | | ++ |
| 102 | ++ | + | | |
| 103 | +++ | + | | |
| 105 | + | ++ | | |
| 109 | +++ | +++ | | |
| 110 | | +++ | | |
| 131 | +++ | +++ | | |
| 132 | ++ | | | |
| 145 | ++ | | | |
| 147 | | + | | |

1. A compound of Formula II, III, IV, V, VI, VII, or VIII: 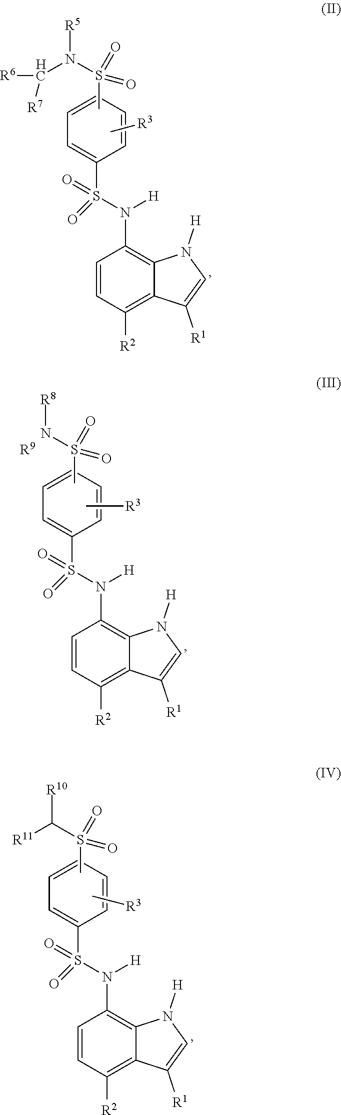 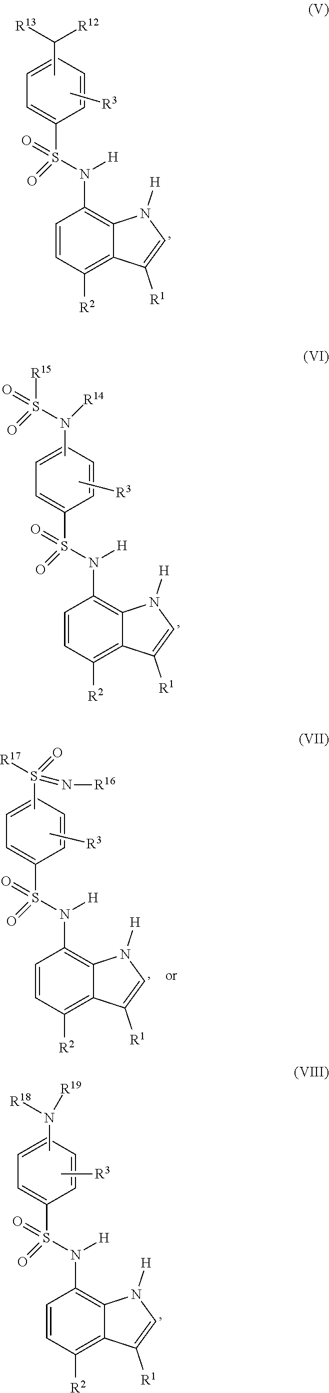
or a pharmaceutically acceptable salt thereof, wherein:
R¹ is selected from the group consisting of chloro, bromo, fluoro, and iodo;
R² is selected from the group consisting of H, chloro, fluoro and methyl;
R³ is selected from the group consisting of H, chloro, fluoro, cyano and methyl;
R⁵ is selected from the group consisting of H and C₁-C₆ alkyl;
R⁶ is selected from the group consisting of —CO₂CH₃, —CH₂OCH₃, CH₂S(O)₂C₁-C₆ alkyl, ((optionally substituted 3 to 7-membered heterocyclyl)oxy)C₁-C₆ alkyl, (optionally substituted C₁-C₆ alkoxy)C₁-C₆ alkyl, amino C₁-C₆ alkyl, optionally substituted C₃-C₇ cycloalkyl, optionally substituted 3 to 7-membered heterocyclyl, optionally substituted C₆-C₁₀ aryl, optionally substituted 5 to 10-membered heteroaryl, (optionally substituted 3 to 7-membered heterocyclyl)C₁-C₆ alkyl, (optionally substituted C₆-C₁₀ aryl)C₁-C₆ alkyl, (optionally substituted 5 to 10-membered heteroaryl)C₁-C₆ alkyl, and 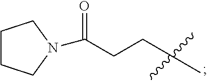 [SMILES: *CCC(=O)N1CCCC1]
R⁷ is selected from the group consisting H, C₁-C₆ alkyl, C₃-C₇ cycloalkyl, and ((dialkylamino)carbonyl)C₁-C₆ alkyl; or
alternatively R⁶ and R⁷ together with the carbon atom to which they are attached form a group selected from an optionally substituted 4 to 7-membered heterocyclyl and optionally substituted indanyl;
R⁸ and R⁹ together with the nitrogen atom to which they are attached form a group selected from optionally substituted 4 to 12-membered heterocyclic ring, or optionally substituted 5 to 6-membered heteroaryl ring, wherein the heterocyclic ring is a monocyclic, fused, spirocyclic or bridged heterocyclyl;
R¹⁰ and R¹¹ together with the carbon atom to which they are attached form an optionally substituted 6-membered heterocyclyl;
R¹² is H;
R¹³ is selected from the group consisting of —NHCOC₁-C₂ alkyl, —CH₂NHCOC₁-C₂ alkyl, —NHCO₂C₁-C₄ alkyl, —CH₂NHCO₂C₁-C₄ alkyl and optionally substituted triazole; or
alternatively R¹² and R¹³ together with the carbon atom to which they are attached form a group selected from optionally substituted C₃-C₆ cycloalkyl, optionally substituted 4 to 6-membered heterocyclyl, optionally substituted phenyl, and optionally substituted 5 to 6-membered-heteroaryl;
R¹⁴ is selected from H and C₁-C₃ alkyl;
R¹⁵ is selected from the group consisting of optionally substituted 6-membered heterocyclyl;
R¹⁶ is selected from the group consisting of H and C₁-C₃ alkyl;
R¹⁷ is selected from the group consisting of C₁-C₆ alkyl, C₆-10 aryl and optionally substituted 6-membered heterocyclyl; and
R¹⁸ and R¹⁹ together with the nitrogen to which they are attached form an optionally substituted 5 to 6-membered heteroaryl group.
with the proviso that the compound is not

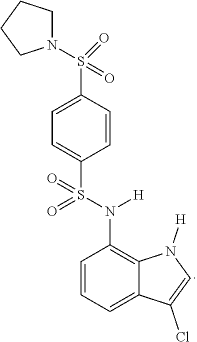 [SMILES: O=S(=O)(Nc1cccc2c(Cl)c[nH]c12)c1ccc(S(=O)(=O)N2CCCC2)cc1]

2. The compound of claim 1 or a pharmaceutically acceptable salt thereof, wherein for the compound of Formula II:
R¹ is chloro;
R² is selected from the group consisting of H and chloro;
R³ is selected from the group consisting H and F;
R⁵ is selected from the group consisting of H and C₁-C₃ alkyl;
R⁶ is selected from the group consisting of CO₂CH₃, —CH₂OCH₃, —CH₂S(O)₂ethyl, phenyl, 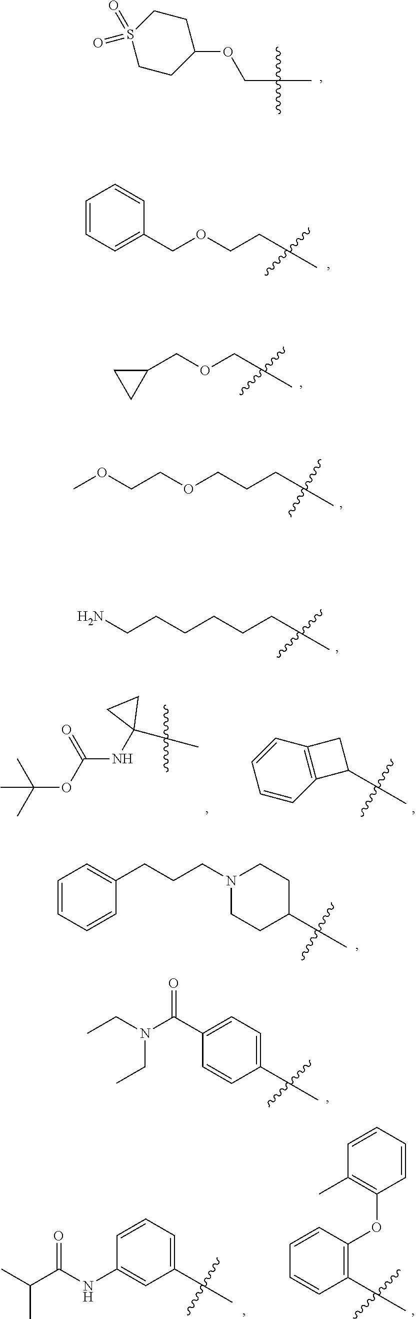 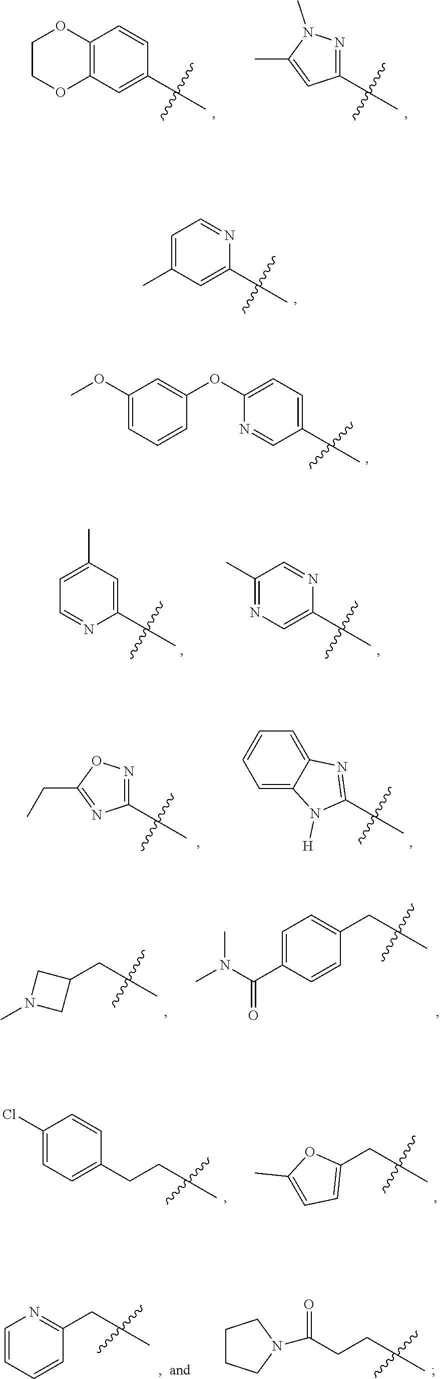
R⁷ is selected from the group consisting H, ethyl, isobutyl, cyclopropyl, and ((dimethylamino)carbonyl)methyl; or
alternatively R⁶ and R⁷ together with the carbon atom to which they are attached form a group selected from

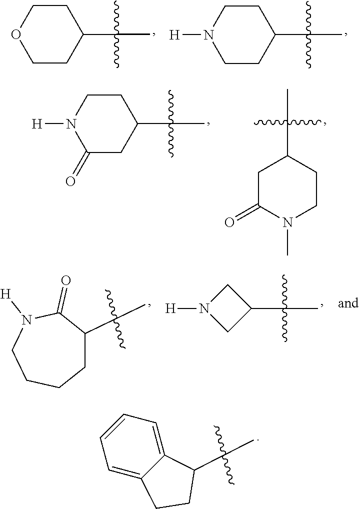

3. A compound according to claim 1 selected from the group consisting of 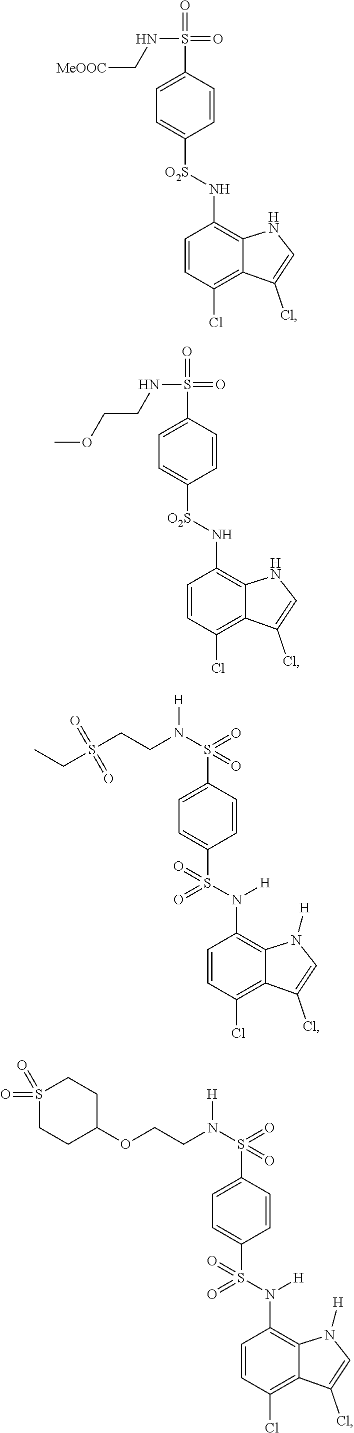 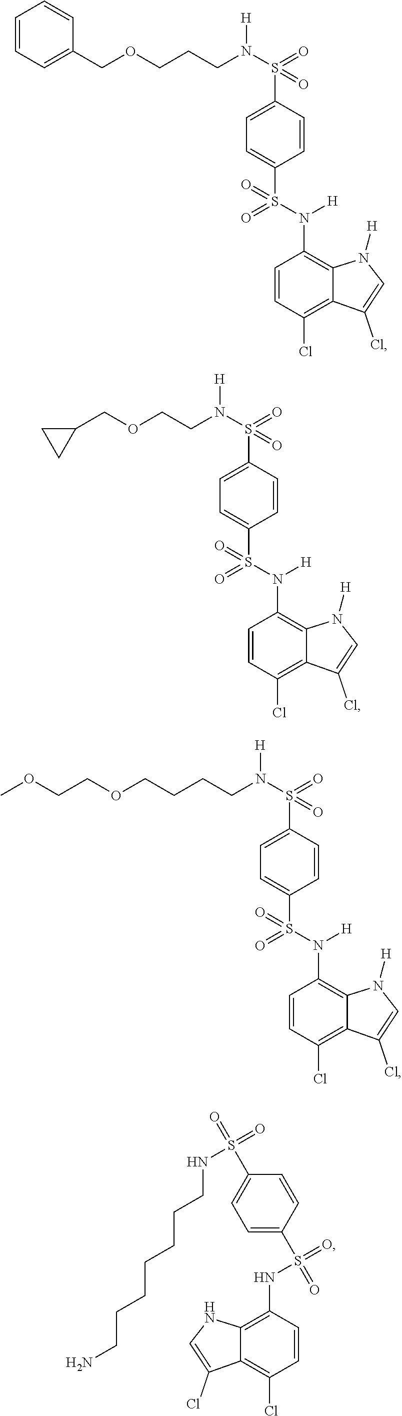 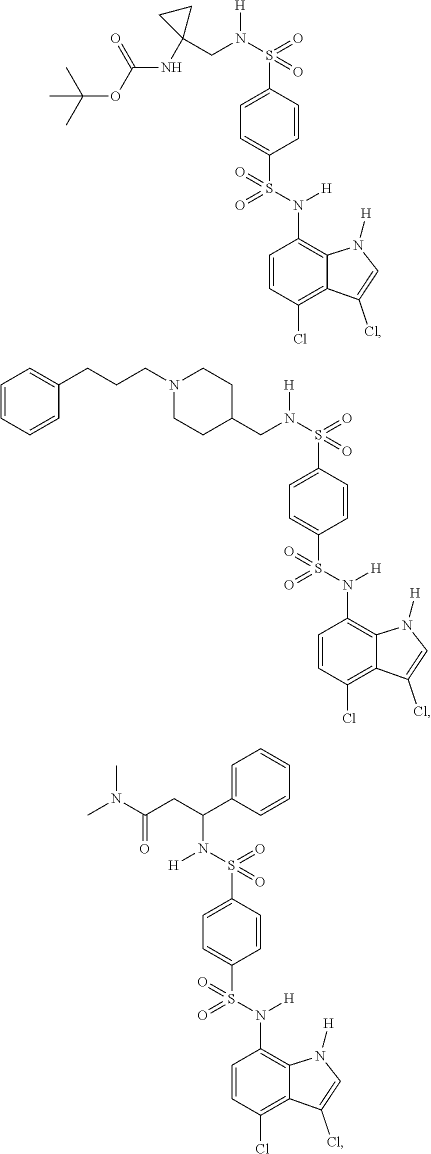 [SMILES: CC(C)(C)OC(=O)NC1(CNS(=O)(=O)c2ccc(S(=O)(=O)Nc3ccc(Cl)c4c(Cl)c[nH]c34)cc2)CC1 ; O=S(=O)(NCC1CCN(CCCc2ccccc2)CC1)c1ccc(S(=O)(=O)Nc2ccc(Cl)c3c(Cl)c[nH]c23)cc1 ; CN(C)C(=O)CC(NS(=O)(=O)c1ccc(S(=O)(=O)Nc2ccc(Cl)c3c(Cl)c[nH]c23)cc1)c1ccccc1] 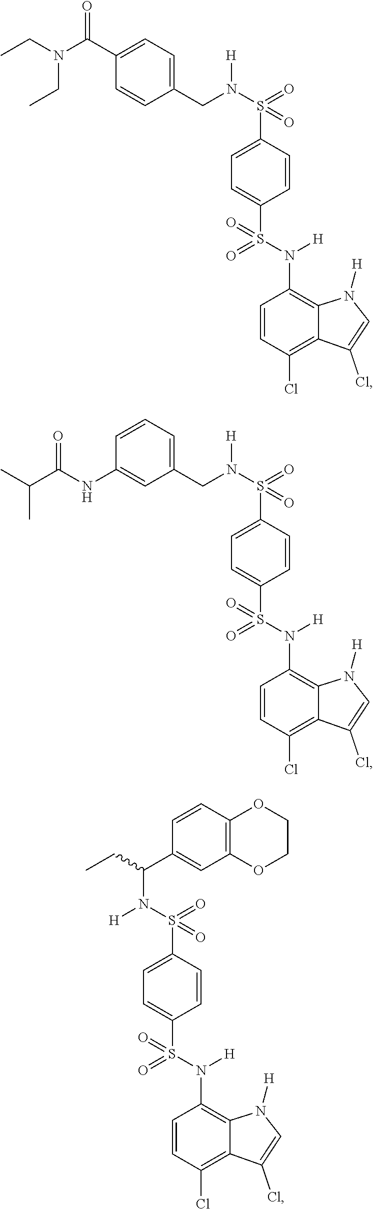 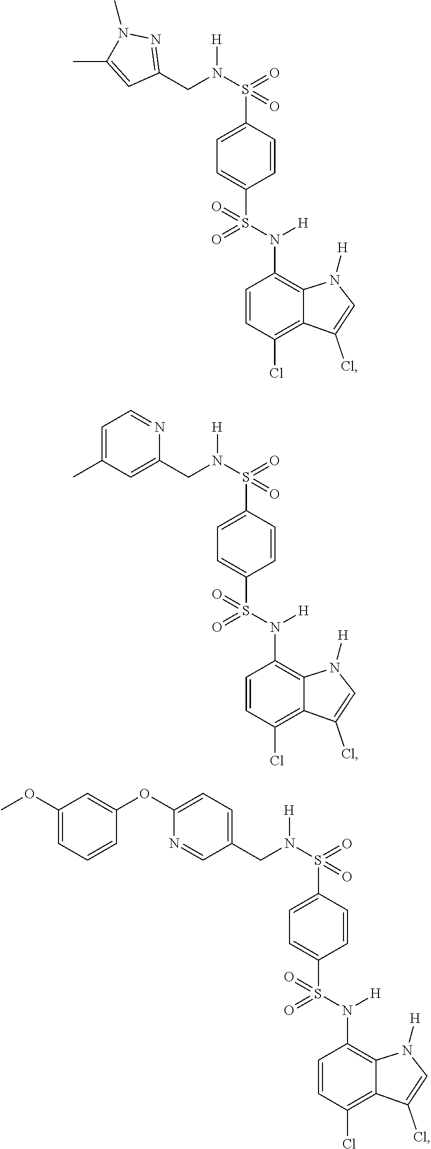 [SMILES: Cc1cc(CNS(=O)(=O)c2ccc(S(=O)(=O)Nc3ccc(Cl)c4c(Cl)c[nH]c34)cc2)nn1C ; Cc1ccnc(CNS(=O)(=O)c2ccc(S(=O)(=O)Nc3ccc(Cl)c4c(Cl)c[nH]c34)cc2)c1 ; COc1cccc(Oc2ccc(CNS(=O)(=O)c3ccc(S(=O)(=O)Nc4ccc(Cl)c5c(Cl)c[nH]c45)cc3)cn2)c1] 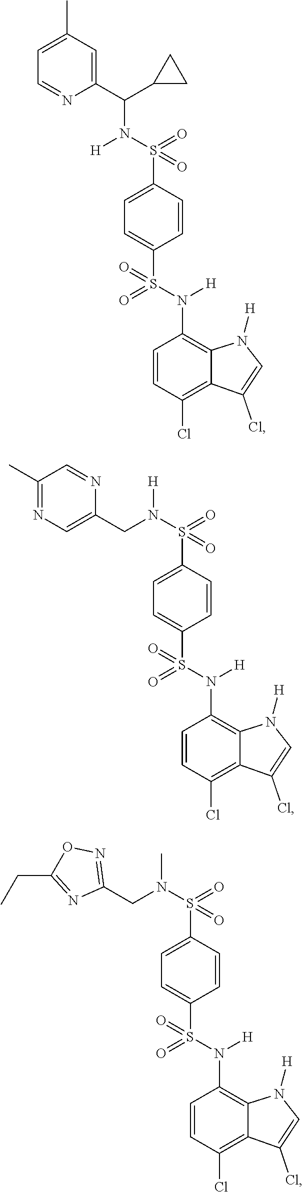 [SMILES: Cc1ccnc(C(NS(=O)(=O)c2ccc(S(=O)(=O)Nc3ccc(Cl)c4c(Cl)c[nH]c34)cc2)C2CC2)c1 ; Cc1cnc(CNS(=O)(=O)c2ccc(S(=O)(=O)Nc3ccc(Cl)c4c(Cl)c[nH]c34)cc2)cn1 ; CCc1nc(CN(C)S(=O)(=O)c2ccc(S(=O)(=O)Nc3ccc(Cl)c4c(Cl)c[nH]c34)cc2)no1] 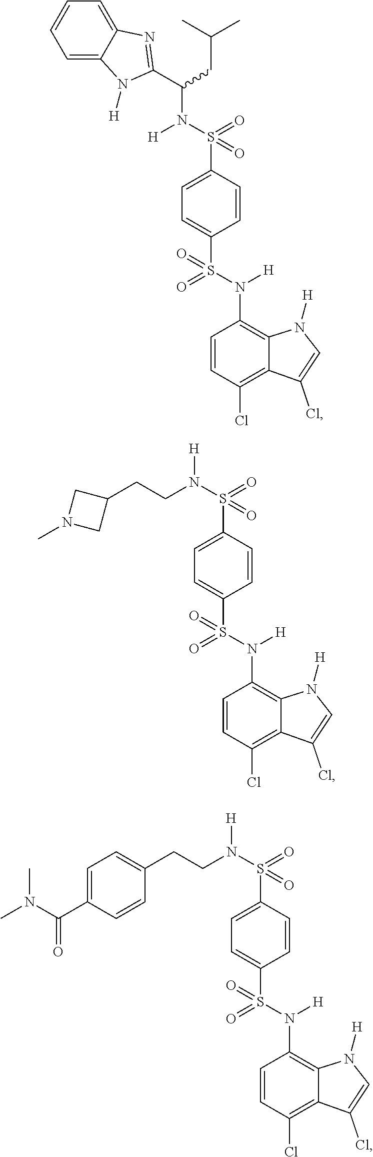 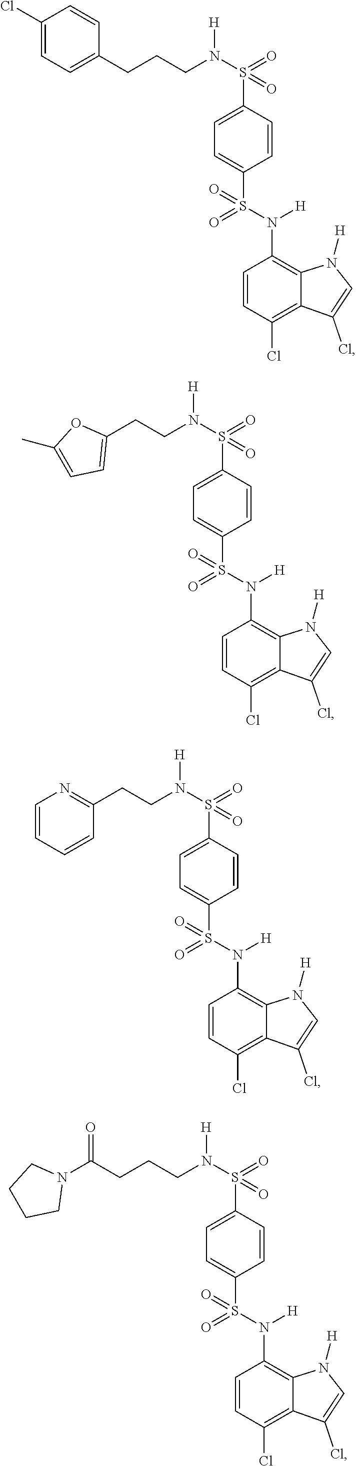 [SMILES: O=S(=O)(NCCCc1ccc(Cl)cc1)c1ccc(S(=O)(=O)Nc2ccc(Cl)c3c(Cl)c[nH]c23)cc1 ; Cc1ccc(CCNS(=O)(=O)c2ccc(S(=O)(=O)Nc3ccc(Cl)c4c(Cl)c[nH]c34)cc2)o1 ; O=S(=O)(NCCc1ccccn1)c1ccc(S(=O)(=O)Nc2ccc(Cl)c3c(Cl)c[nH]c23)cc1 ; O=C(CCCNS(=O)(=O)c1ccc(S(=O)(=O)Nc2ccc(Cl)c3c(Cl)c[nH]c23)cc1)N1CCCC1] 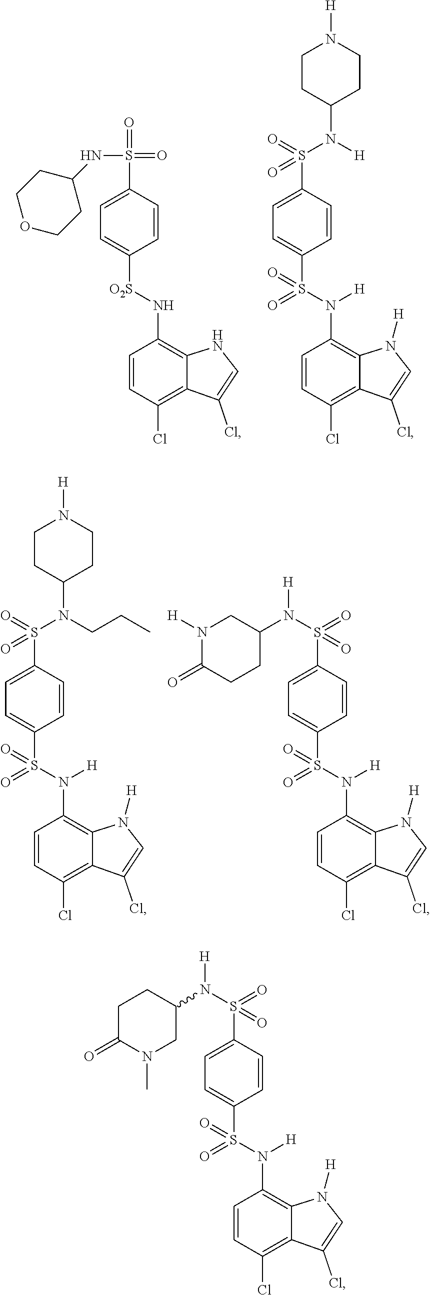 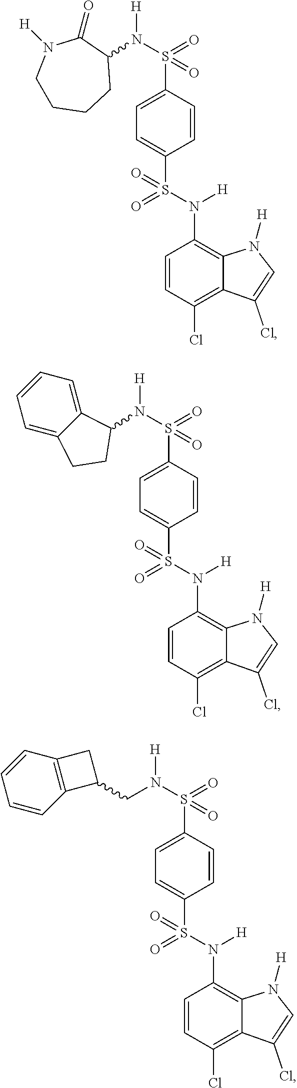 [SMILES: O=C1NCCCCC1NS(=O)(=O)c1ccc(S(=O)(=O)Nc2ccc(Cl)c3c(Cl)c[nH]c23)cc1 ; O=S(=O)(Nc1ccc(Cl)c2c(Cl)c[nH]c12)c1ccc(S(=O)(=O)NC2CCc3ccccc32)cc1 ; O=S(=O)(NCC1Cc2ccccc21)c1ccc(S(=O)(=O)Nc2ccc(Cl)c3c(Cl)c[nH]c23)cc1] 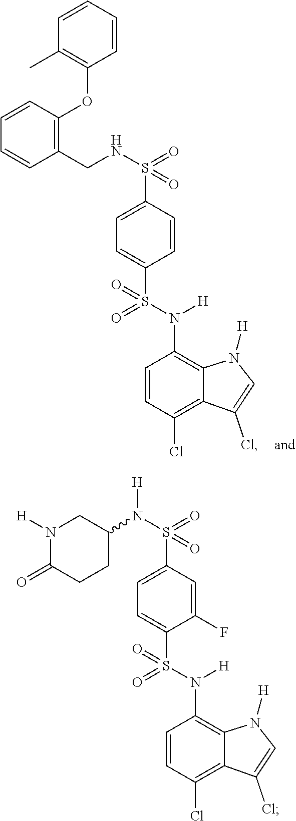 [SMILES: Cc1ccccc1Oc1ccccc1CNS(=O)(=O)c1ccc(S(=O)(=O)Nc2ccc(Cl)c3c(Cl)c[nH]c23)cc1 ; O=C1CCC(NS(=O)(=O)c2ccc(S(=O)(=O)Nc3ccc(Cl)c4c(Cl)c[nH]c34)c(F)c2)CN1]
or a pharmaceutically acceptable salt thereof.

4. (canceled)

5. The compound of claim 1, or a pharmaceutically acceptable salt thereof wherein for the compound of Formula III:
R¹ is chloro;
R² is selected from the group consisting of H and chloro;
R³ is selected from the group consisting H, F and cyano; and
R⁸ and R⁹ together with the nitrogen atom to which they are attached form a group selected from piperidinyl,

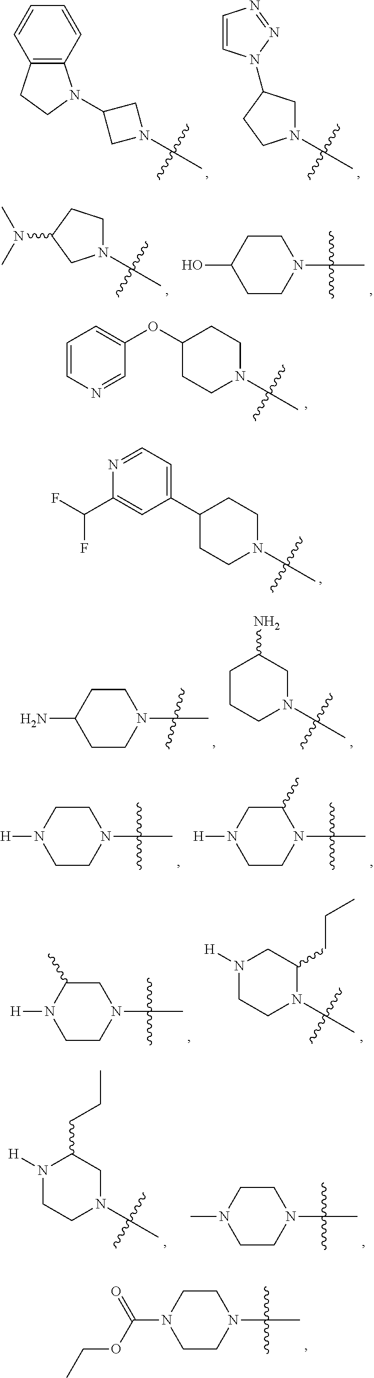

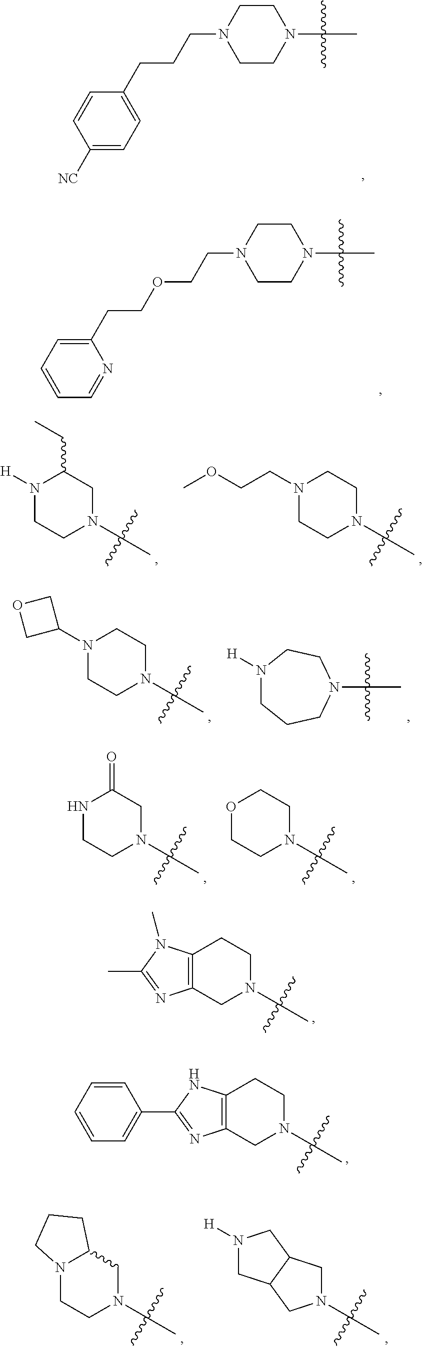

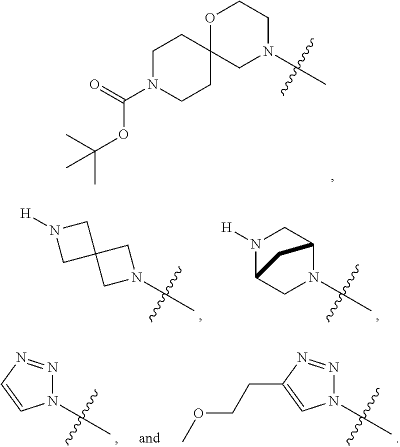

6. A compound according to claim 1 selected from the group consisting of 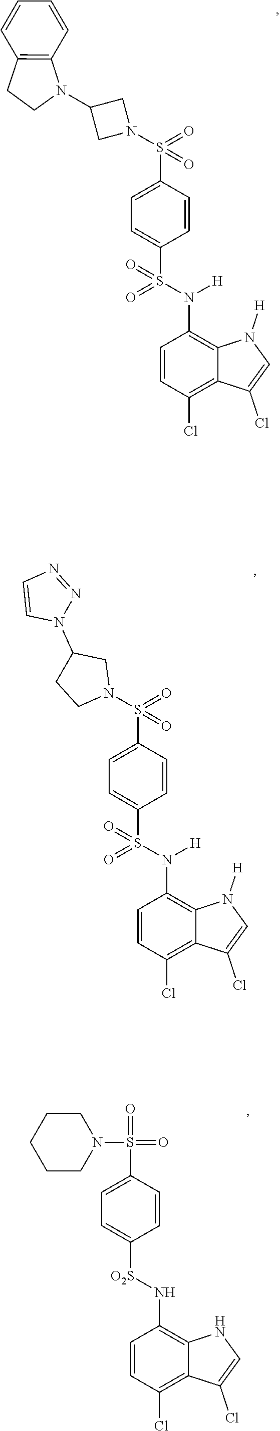 [SMILES: O=S(=O)(Nc1ccc(Cl)c2c(Cl)c[nH]c12)c1ccc(S(=O)(=O)N2CC(N3CCc4ccccc43)C2)cc1 ; O=S(=O)(Nc1ccc(Cl)c2c(Cl)c[nH]c12)c1ccc(S(=O)(=O)N2CCC(n3ccnn3)C2)cc1 ; O=S(=O)(Nc1ccc(Cl)c2c(Cl)c[nH]c12)c1ccc(S(=O)(=O)N2CCCCC2)cc1] 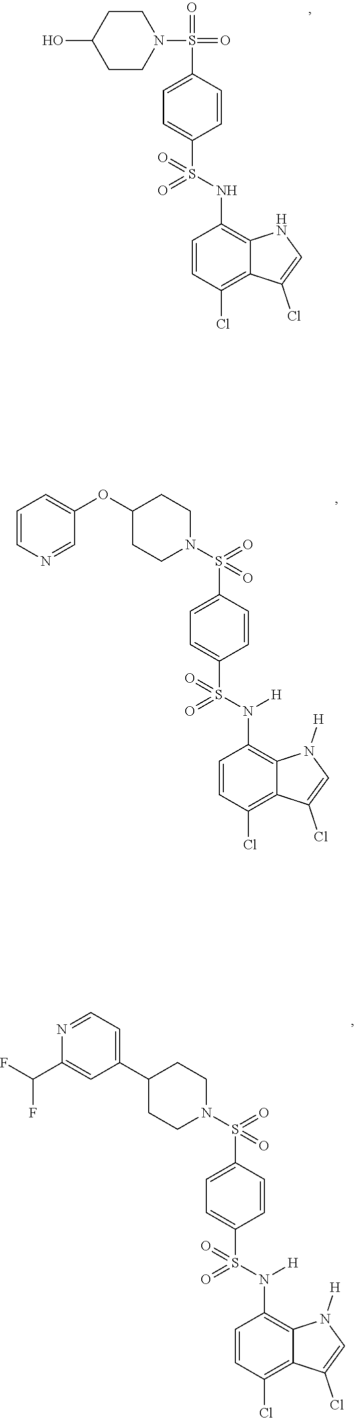 [SMILES: O=S(=O)(Nc1ccc(Cl)c2c(Cl)c[nH]c12)c1ccc(S(=O)(=O)N2CCC(O)CC2)cc1 ; O=S(=O)(Nc1ccc(Cl)c2c(Cl)c[nH]c12)c1ccc(S(=O)(=O)N2CCC(Oc3cccnc3)CC2)cc1 ; O=S(=O)(Nc1ccc(Cl)c2c(Cl)c[nH]c12)c1ccc(S(=O)(=O)N2CCC(c3ccnc(C(F)F)c3)CC2)cc1] 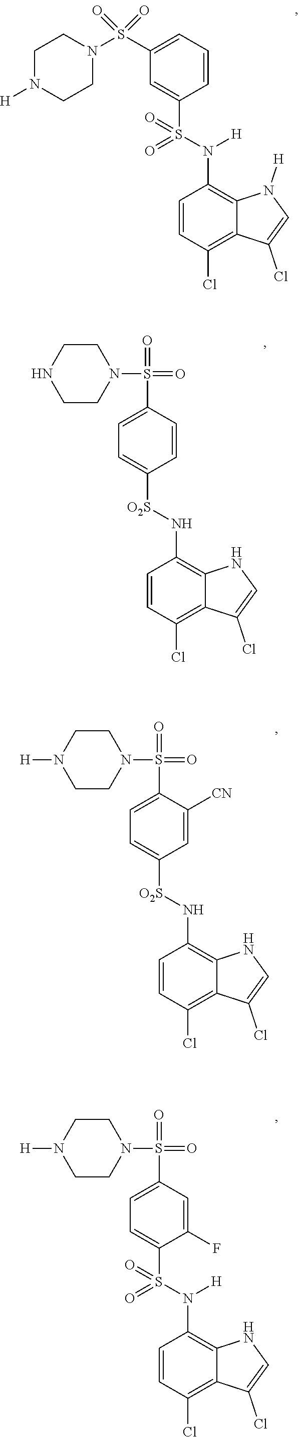 [SMILES: O=S(=O)(Nc1ccc(Cl)c2c(Cl)c[nH]c12)c1cccc(S(=O)(=O)N2CCNCC2)c1 ; O=S(=O)(Nc1ccc(Cl)c2c(Cl)c[nH]c12)c1ccc(S(=O)(=O)N2CCNCC2)cc1 ; N#Cc1cc(S(=O)(=O)Nc2ccc(Cl)c3c(Cl)c[nH]c23)ccc1S(=O)(=O)N1CCNCC1 ; O=S(=O)(Nc1ccc(Cl)c2c(Cl)c[nH]c12)c1ccc(S(=O)(=O)N2CCNCC2)cc1F] 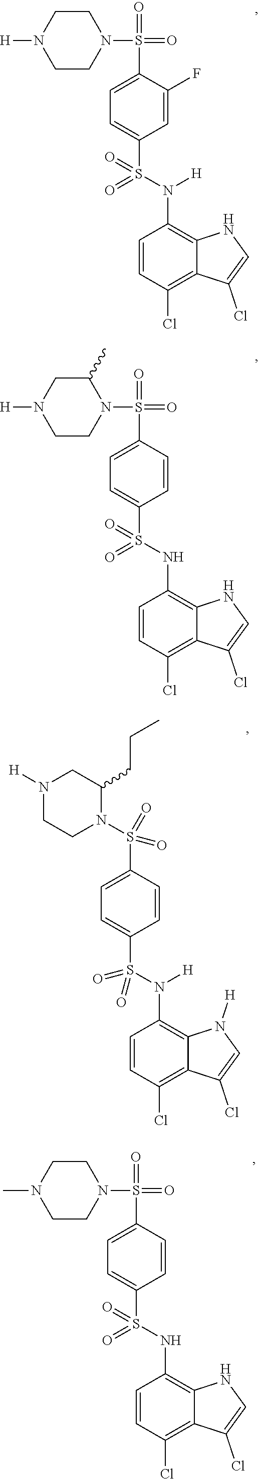 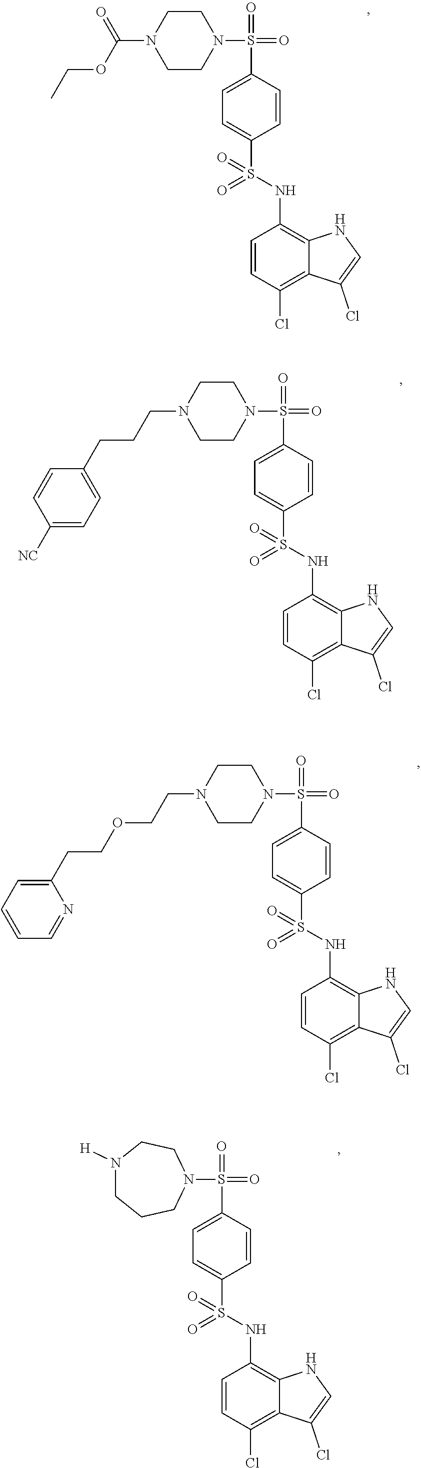 [SMILES: CCOC(=O)N1CCN(S(=O)(=O)c2ccc(S(=O)(=O)Nc3ccc(Cl)c4c(Cl)c[nH]c34)cc2)CC1 ; N#Cc1ccc(CCCN2CCN(S(=O)(=O)c3ccc(S(=O)(=O)Nc4ccc(Cl)c5c(Cl)c[nH]c45)cc3)CC2)cc1 ; O=S(=O)(Nc1ccc(Cl)c2c(Cl)c[nH]c12)c1ccc(S(=O)(=O)N2CCN(CCOCCc3ccccn3)CC2)cc1 ; O=S(=O)(Nc1ccc(Cl)c2c(Cl)c[nH]c12)c1ccc(S(=O)(=O)N2CCCNCC2)cc1] 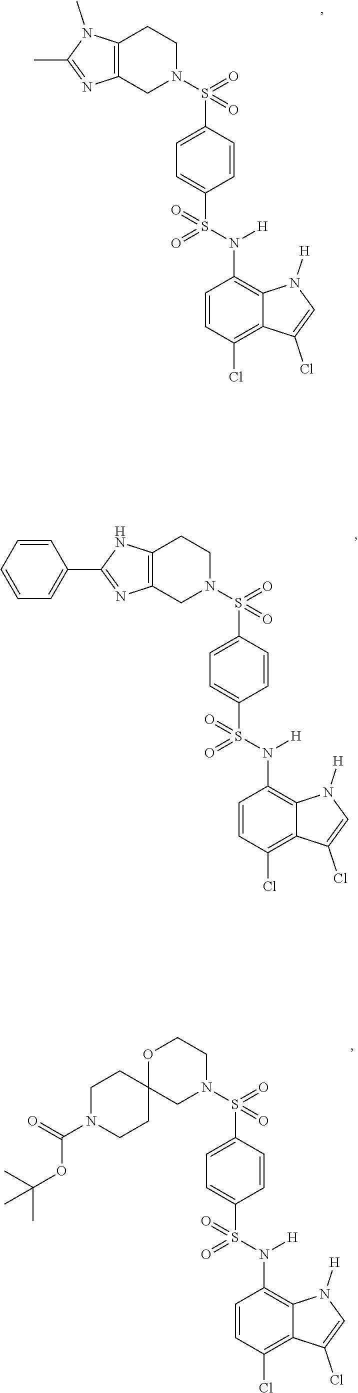 [SMILES: Cc1nc2c(n1C)CCN(S(=O)(=O)c1ccc(S(=O)(=O)Nc3ccc(Cl)c4c(Cl)c[nH]c34)cc1)C2 ; O=S(=O)(Nc1ccc(Cl)c2c(Cl)c[nH]c12)c1ccc(S(=O)(=O)N2CCc3[nH]c(-c4ccccc4)nc3C2)cc1 ; CC(C)(C)OC(=O)N1CCC2(CC1)CN(S(=O)(=O)c1ccc(S(=O)(=O)Nc3ccc(Cl)c4c(Cl)c[nH]c34)cc1)CCO2] 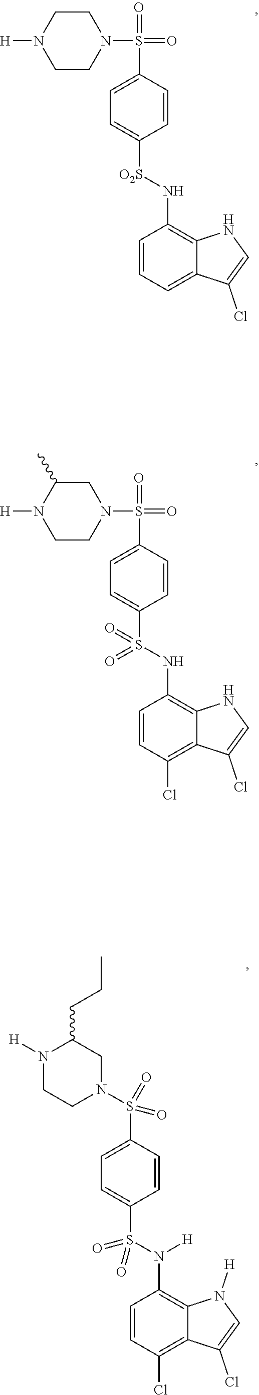 [SMILES: O=S(=O)(Nc1cccc2c(Cl)c[nH]c12)c1ccc(S(=O)(=O)N2CCNCC2)cc1 ; CC1CN(S(=O)(=O)c2ccc(S(=O)(=O)Nc3ccc(Cl)c4c(Cl)c[nH]c34)cc2)CCN1 ; CCCC1CN(S(=O)(=O)c2ccc(S(=O)(=O)Nc3ccc(Cl)c4c(Cl)c[nH]c34)cc2)CCN1] 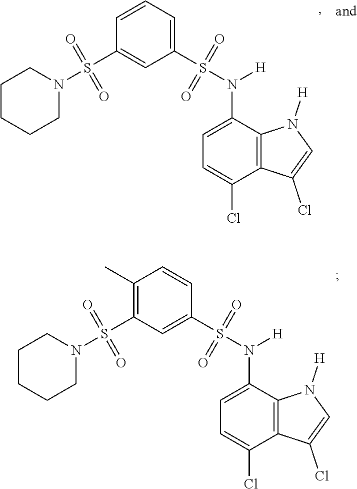 [SMILES: O=S(=O)(Nc1ccc(Cl)c2c(Cl)c[nH]c12)c1cccc(S(=O)(=O)N2CCCCC2)c1 ; Cc1ccc(S(=O)(=O)Nc2ccc(Cl)c3c(Cl)c[nH]c23)cc1S(=O)(=O)N1CCCCC1]
or a pharmaceutically acceptable salt thereof.

7. (canceled)

8. The compound of claim 1 or a pharmaceutically acceptable salt thereof, wherein for the compound of Formula IV:
R¹ is chloro;
R² is selected from the group consisting of H and chloro;
R³ is selected from the group consisting H and F; and
R¹⁰ and R¹¹ together with the carbon atom to which they are attached form

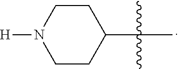 [SMILES: *C1CCNCC1]

9. A compound according to claim 1 selected from the group consisting of 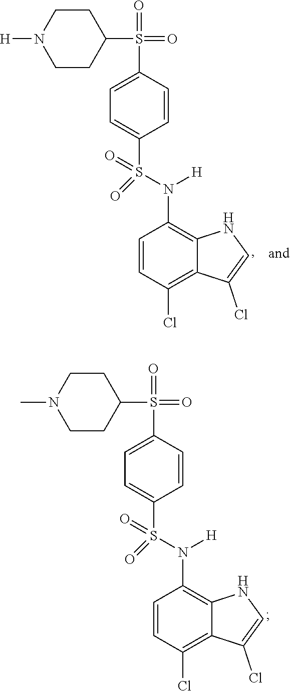 [SMILES: O=S(=O)(Nc1ccc(Cl)c2c(Cl)c[nH]c12)c1ccc(S(=O)(=O)C2CCNCC2)cc1 ; CN1CCC(S(=O)(=O)c2ccc(S(=O)(=O)Nc3ccc(Cl)c4c(Cl)c[nH]c34)cc2)CC1]
or a pharmaceutically acceptable salt thereof.

10. (canceled)

11. The compound of claim 1 or a pharmaceutically acceptable salt thereof, wherein for the compound of Formula V:
R¹ is chloro;
R² is selected from the group consisting of H and chloro;
R³ is selected from the group consisting of H, chloro, fluoro and methyl;
R¹² is H;
R¹³ is selected from the group consisting of —NHCOCH₂CH₃, —CH₂NHCOCH₃, —NHCO₂t-Bu, —CH₂NHCO₂t-Bu, and 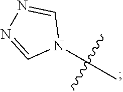 [SMILES: *n1cnnc1]
or
alternatively, R¹² and R¹³ together with the carbon atom to which they are attached form a group selected from cyclohexyl, phenyl,

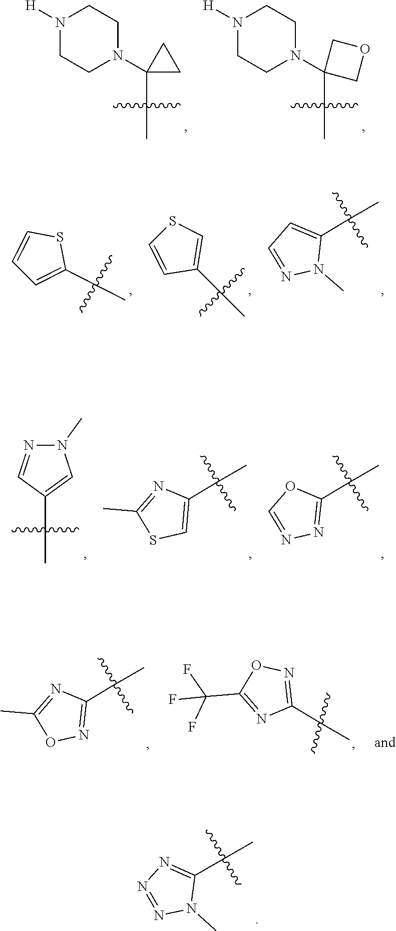

12. A compound according to claim 1 selected from the group consisting of 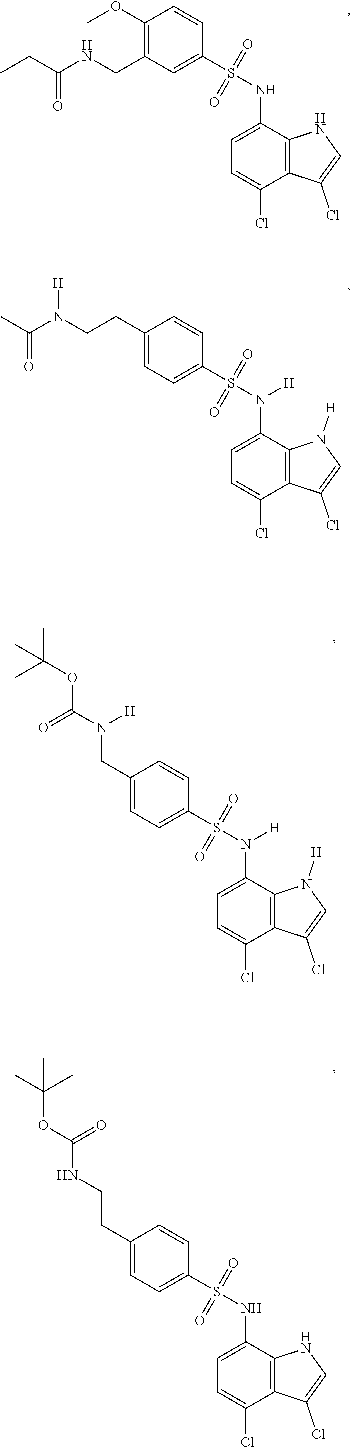 [SMILES: CCC(=O)NCc1cc(S(=O)(=O)Nc2ccc(Cl)c3c(Cl)c[nH]c23)ccc1OC ; CC(=O)NCCc1ccc(S(=O)(=O)Nc2ccc(Cl)c3c(Cl)c[nH]c23)cc1 ; CC(C)(C)OC(=O)NCc1ccc(S(=O)(=O)Nc2ccc(Cl)c3c(Cl)c[nH]c23)cc1 ; CC(C)(C)OC(=O)NCCc1ccc(S(=O)(=O)Nc2ccc(Cl)c3c(Cl)c[nH]c23)cc1] 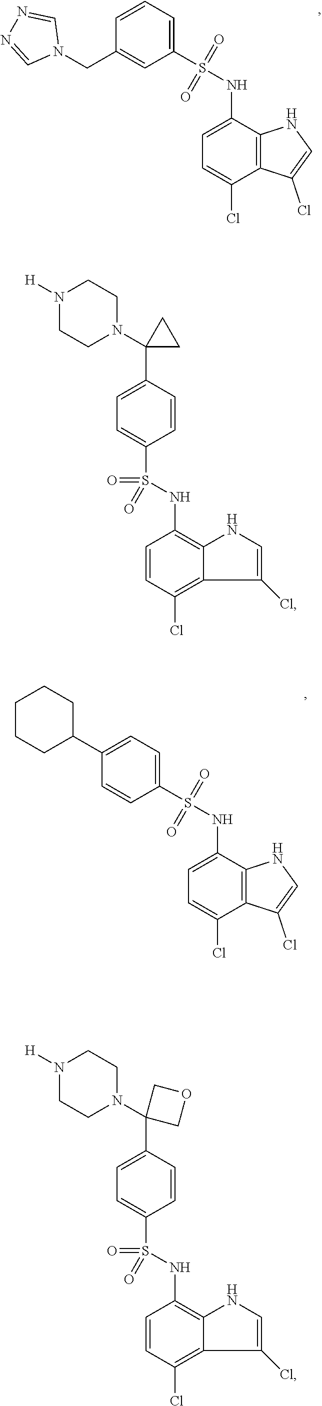 [SMILES: O=S(=O)(Nc1ccc(Cl)c2c(Cl)c[nH]c12)c1cccc(Cn2cnnc2)c1 ; O=S(=O)(Nc1ccc(Cl)c2c(Cl)c[nH]c12)c1ccc(C2(N3CCNCC3)CC2)cc1 ; O=S(=O)(Nc1ccc(Cl)c2c(Cl)c[nH]c12)c1ccc(C2CCCCC2)cc1 ; O=S(=O)(Nc1ccc(Cl)c2c(Cl)c[nH]c12)c1ccc(C2(N3CCNCC3)COC2)cc1] 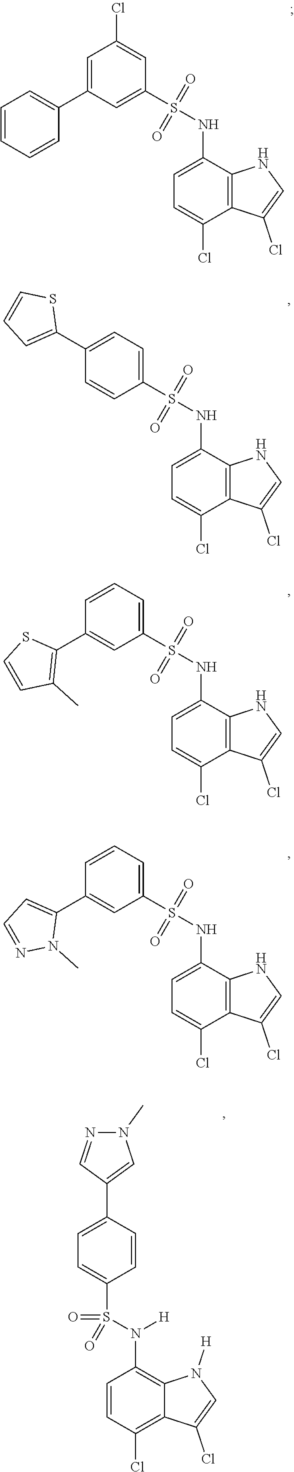 [SMILES: O=S(=O)(Nc1ccc(Cl)c2c(Cl)c[nH]c12)c1cc(Cl)cc(-c2ccccc2)c1 ; O=S(=O)(Nc1ccc(Cl)c2c(Cl)c[nH]c12)c1ccc(-c2cccs2)cc1 ; Cc1ccsc1-c1cccc(S(=O)(=O)Nc2ccc(Cl)c3c(Cl)c[nH]c23)c1 ; Cn1nccc1-c1cccc(S(=O)(=O)Nc2ccc(Cl)c3c(Cl)c[nH]c23)c1 ; Cn1cc(-c2ccc(S(=O)(=O)Nc3ccc(Cl)c4c(Cl)c[nH]c34)cc2)cn1] 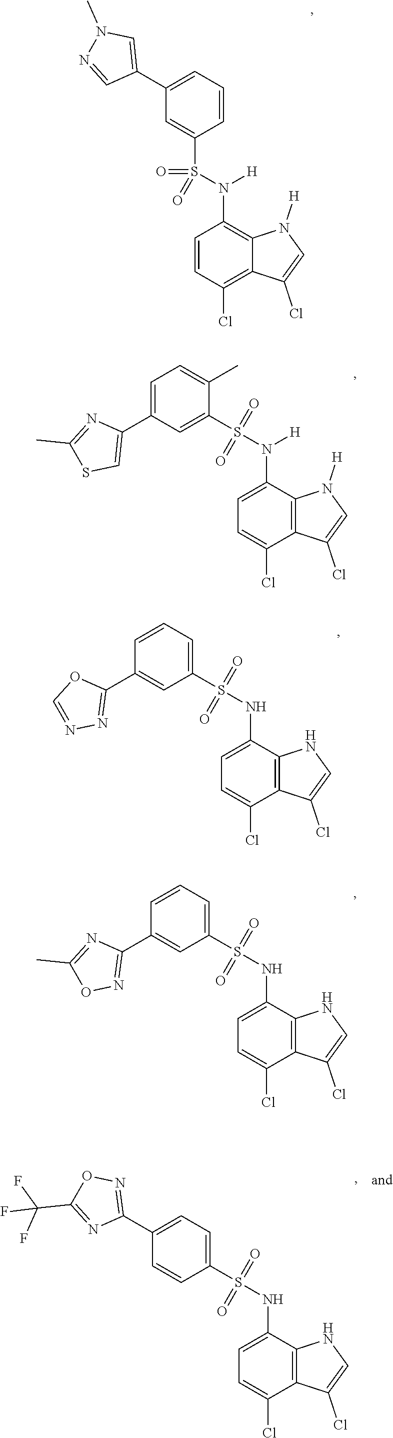 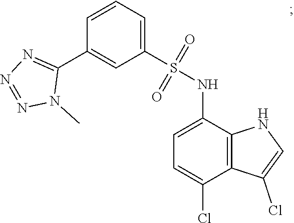 [SMILES: Cn1nnnc1-c1cccc(S(=O)(=O)Nc2ccc(Cl)c3c(Cl)c[nH]c23)c1]
or a pharmaceutically acceptable salt thereof.

13. (canceled)

14. The compound of claim 1 or a pharmaceutically acceptable salt thereof, wherein for the compound of Formula VI:
R¹ is chloro;
R² is selected from the group consisting of H and chloro;
R³ is selected from the group consisting H and F;
R¹⁴ is selected from the group consisting of H and propyl; and
R¹⁵ is selected from the group consisting of

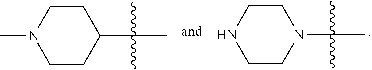

15. A compound according to claim 1 selected from the group consisting of 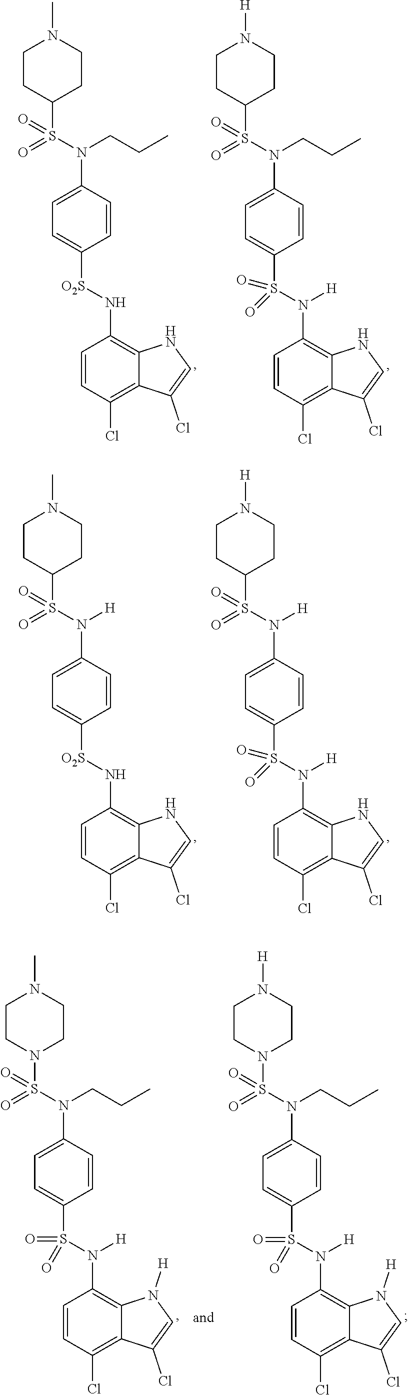 [SMILES: CCCN(c1ccc(S(=O)(=O)Nc2ccc(Cl)c3c(Cl)c[nH]c23)cc1)S(=O)(=O)C1CCN(C)CC1 ; CCCN(c1ccc(S(=O)(=O)Nc2ccc(Cl)c3c(Cl)c[nH]c23)cc1)S(=O)(=O)C1CCNCC1 ; CN1CCC(S(=O)(=O)Nc2ccc(S(=O)(=O)Nc3ccc(Cl)c4c(Cl)c[nH]c34)cc2)CC1 ; O=S(=O)(Nc1ccc(Cl)c2c(Cl)c[nH]c12)c1ccc(NS(=O)(=O)C2CCNCC2)cc1 ; CCCN(c1ccc(S(=O)(=O)Nc2ccc(Cl)c3c(Cl)c[nH]c23)cc1)S(=O)(=O)N1CCN(C)CC1 ; CCCN(c1ccc(S(=O)(=O)Nc2ccc(Cl)c3c(Cl)c[nH]c23)cc1)S(=O)(=O)N1CCNCC1]
or a pharmaceutically acceptable salt thereof.

16. (canceled)

17. The compound of claim 1 or a pharmaceutically acceptable salt thereof, wherein for the compound of Formula VII:
R¹ is chloro;
R² is selected from the group consisting of H and chloro;
R³ is selected from the group consisting H and F;
R¹⁶ is ethyl; and
R⁷ is selected from the group consisting of

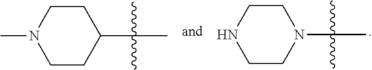

18. A compound according to claim 1 selected from the group consisting of 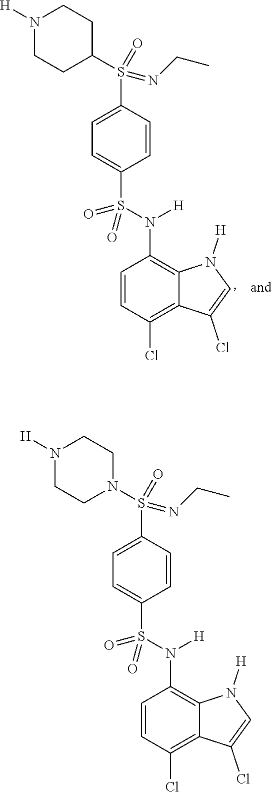 [SMILES: CCN=S(=O)(c1ccc(S(=O)(=O)Nc2ccc(Cl)c3c(Cl)c[nH]c23)cc1)C1CCNCC1 ; CCN=S(=O)(c1ccc(S(=O)(=O)Nc2ccc(Cl)c3c(Cl)c[nH]c23)cc1)N1CCNCC1]
or a pharmaceutically acceptable salt thereof.

19. (canceled)

20. A compound according to claim 1 or a pharmaceutically acceptable salt thereof, wherein for the compound of Formula VIII:
R¹ is chloro;
R² is selected from the group consisting of H and chloro;
R³ is selected from the group consisting H and F; and
R¹⁸ and R¹⁹ together with the nitrogen to which they are attached form a group selected from

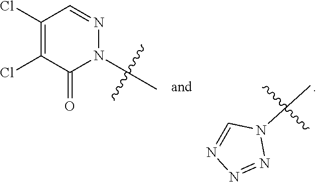 [SMILES: *n1ncc(Cl)c(Cl)c1=O ; *n1cnnn1]

21. A compound according to claim 1 selected from the group consisting of 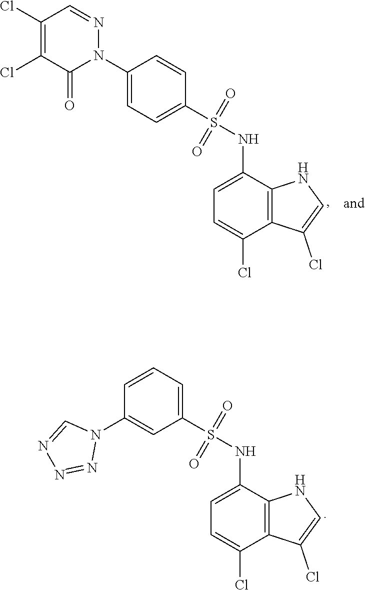 [SMILES: O=c1c(Cl)c(Cl)cnn1-c1ccc(S(=O)(=O)Nc2ccc(Cl)c3c(Cl)c[nH]c23)cc1 ; O=S(=O)(Nc1ccc(Cl)c2c(Cl)c[nH]c12)c1cccc(-n2cnnn2)c1]
or a pharmaceutically acceptable salt thereof.

22. A compound selected from the group consisting of 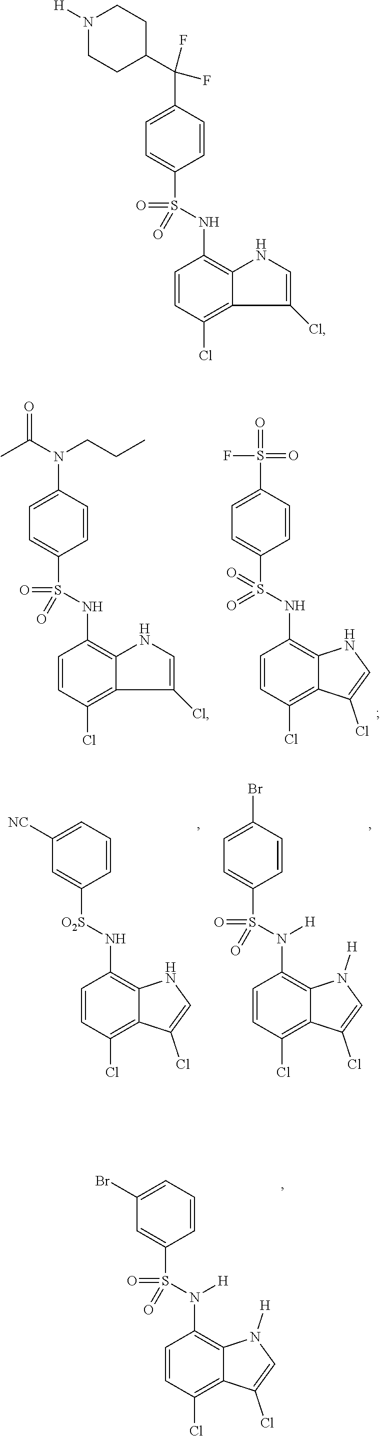 [SMILES: O=S(=O)(Nc1ccc(Cl)c2c(Cl)c[nH]c12)c1ccc(C(F)(F)C2CCNCC2)cc1 ; CCCN(C(C)=O)c1ccc(S(=O)(=O)Nc2ccc(Cl)c3c(Cl)c[nH]c23)cc1 ; O=S(=O)(F)c1ccc(S(=O)(=O)Nc2ccc(Cl)c3c(Cl)c[nH]c23)cc1 ; N#Cc1cccc(S(=O)(=O)Nc2ccc(Cl)c3c(Cl)c[nH]c23)c1 ; O=S(=O)(Nc1ccc(Cl)c2c(Cl)c[nH]c12)c1ccc(Br)cc1 ; O=S(=O)(Nc1ccc(Cl)c2c(Cl)c[nH]c12)c1cccc(Br)c1] 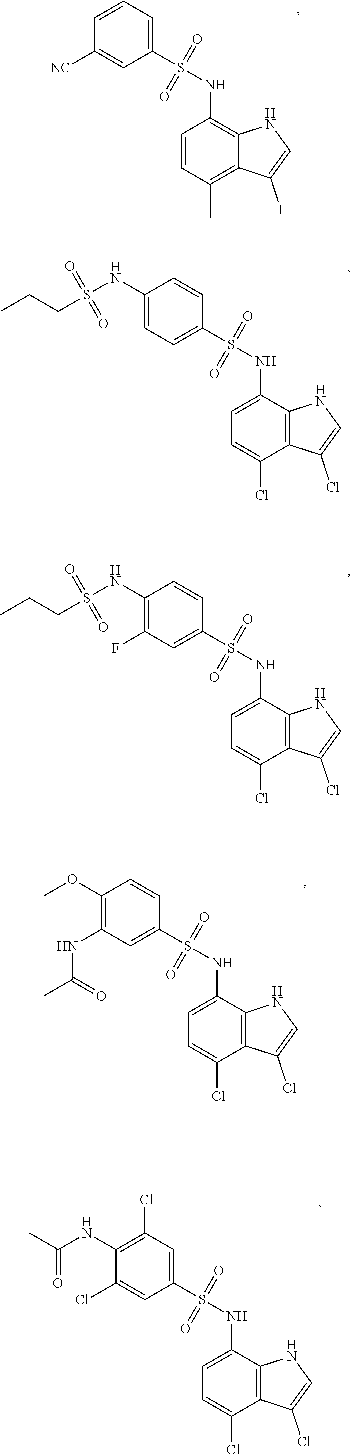 [SMILES: Cc1ccc(NS(=O)(=O)c2cccc(C#N)c2)c2[nH]cc(I)c12 ; CCCS(=O)(=O)Nc1ccc(S(=O)(=O)Nc2ccc(Cl)c3c(Cl)c[nH]c23)cc1 ; CCCS(=O)(=O)Nc1ccc(S(=O)(=O)Nc2ccc(Cl)c3c(Cl)c[nH]c23)cc1F ; COc1ccc(S(=O)(=O)Nc2ccc(Cl)c3c(Cl)c[nH]c23)cc1NC(C)=O ; CC(=O)Nc1c(Cl)cc(S(=O)(=O)Nc2ccc(Cl)c3c(Cl)c[nH]c23)cc1Cl] 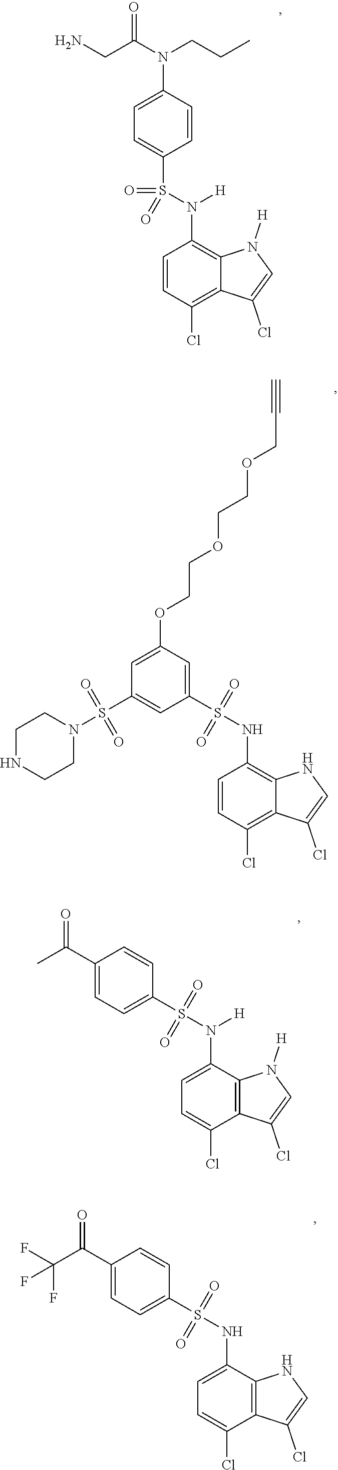 [SMILES: CCCN(C(=O)CN)c1ccc(S(=O)(=O)Nc2ccc(Cl)c3c(Cl)c[nH]c23)cc1 ; C#CCOCCOCCOc1cc(S(=O)(=O)Nc2ccc(Cl)c3c(Cl)c[nH]c23)cc(S(=O)(=O)N2CCNCC2)c1 ; CC(=O)c1ccc(S(=O)(=O)Nc2ccc(Cl)c3c(Cl)c[nH]c23)cc1 ; O=C(c1ccc(S(=O)(=O)Nc2ccc(Cl)c3c(Cl)c[nH]c23)cc1)C(F)(F)F] 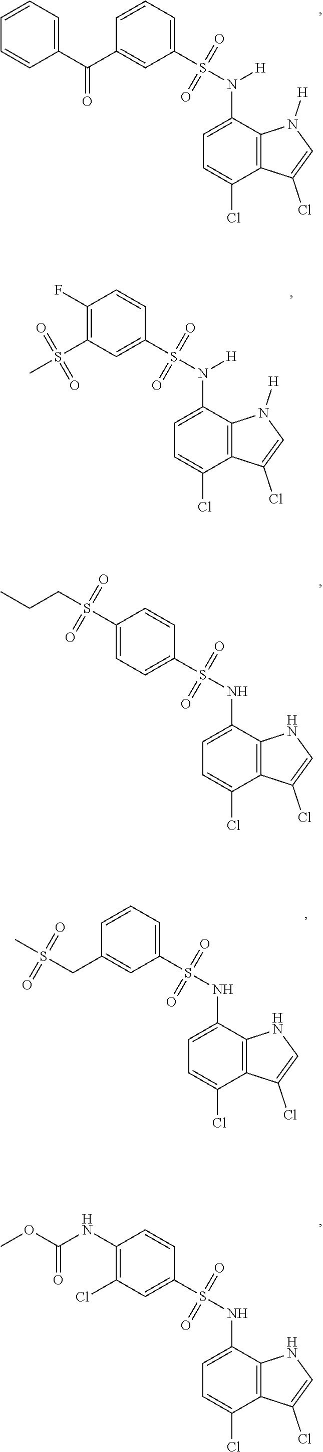 [SMILES: O=C(c1ccccc1)c1cccc(S(=O)(=O)Nc2ccc(Cl)c3c(Cl)c[nH]c23)c1 ; CS(=O)(=O)c1cc(S(=O)(=O)Nc2ccc(Cl)c3c(Cl)c[nH]c23)ccc1F ; CCCS(=O)(=O)c1ccc(S(=O)(=O)Nc2ccc(Cl)c3c(Cl)c[nH]c23)cc1 ; CS(=O)(=O)Cc1cccc(S(=O)(=O)Nc2ccc(Cl)c3c(Cl)c[nH]c23)c1 ; COC(=O)Nc1ccc(S(=O)(=O)Nc2ccc(Cl)c3c(Cl)c[nH]c23)cc1Cl] 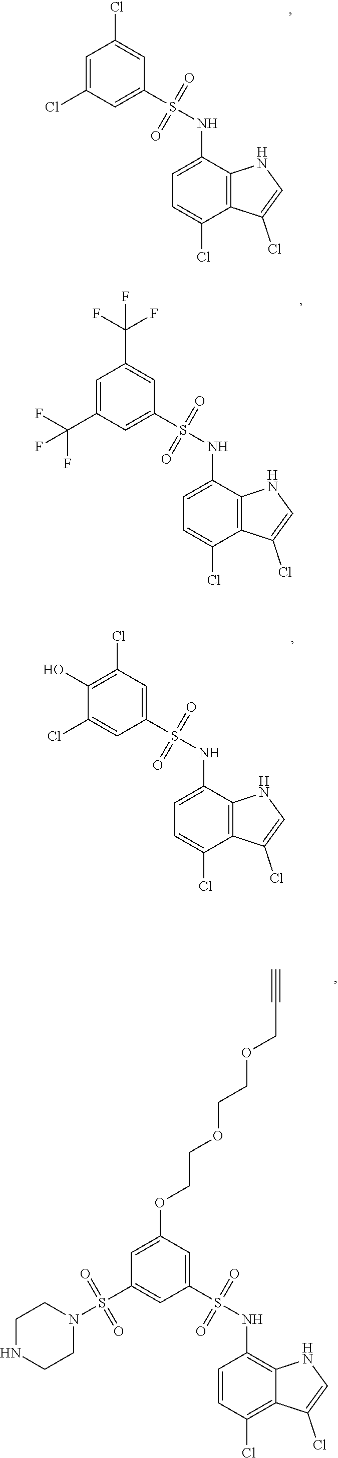 [SMILES: O=S(=O)(Nc1ccc(Cl)c2c(Cl)c[nH]c12)c1cc(Cl)cc(Cl)c1 ; O=S(=O)(Nc1ccc(Cl)c2c(Cl)c[nH]c12)c1cc(C(F)(F)F)cc(C(F)(F)F)c1 ; O=S(=O)(Nc1ccc(Cl)c2c(Cl)c[nH]c12)c1cc(Cl)c(O)c(Cl)c1 ; C#CCOCCOCCOc1cc(S(=O)(=O)Nc2ccc(Cl)c3c(Cl)c[nH]c23)cc(S(=O)(=O)N2CCNCC2)c1] 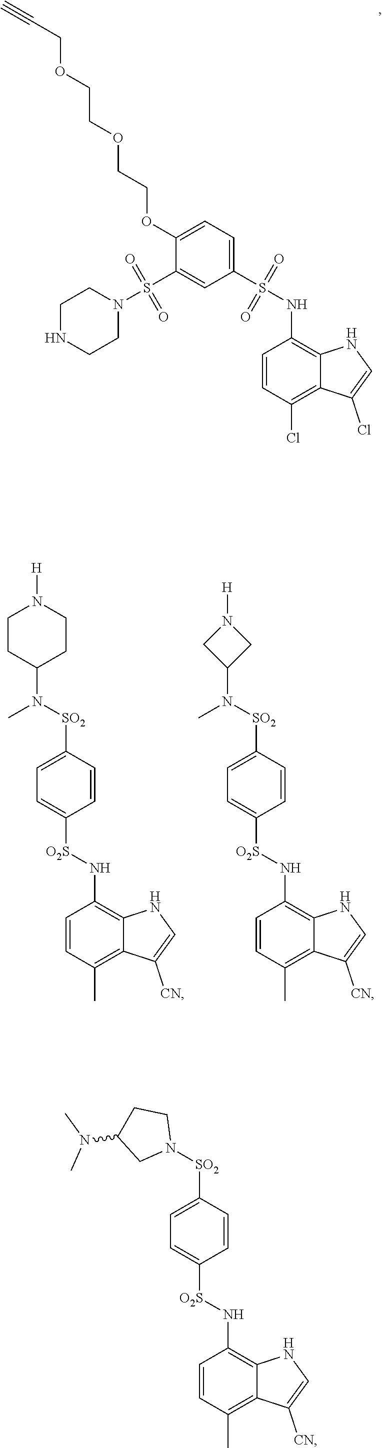 [SMILES: C#CCOCCOCCOc1ccc(S(=O)(=O)Nc2ccc(Cl)c3c(Cl)c[nH]c23)cc1S(=O)(=O)N1CCNCC1 ; Cc1ccc(NS(=O)(=O)c2ccc(S(=O)(=O)N(C)C3CCNCC3)cc2)c2[nH]cc(C#N)c12 ; Cc1ccc(NS(=O)(=O)c2ccc(S(=O)(=O)N(C)C3CNC3)cc2)c2[nH]cc(C#N)c12 ; Cc1ccc(NS(=O)(=O)c2ccc(S(=O)(=O)N3CCC(N(C)C)C3)cc2)c2[nH]cc(C#N)c12] 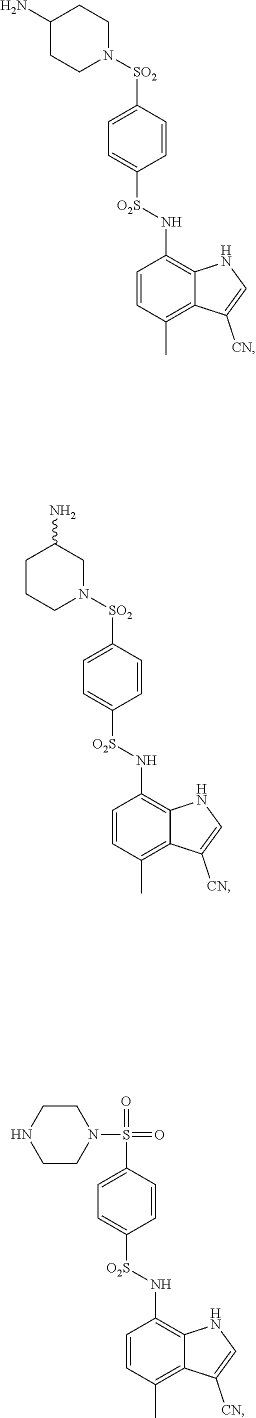 [SMILES: Cc1ccc(NS(=O)(=O)c2ccc(S(=O)(=O)N3CCC(N)CC3)cc2)c2[nH]cc(C#N)c12 ; Cc1ccc(NS(=O)(=O)c2ccc(S(=O)(=O)N3CCCC(N)C3)cc2)c2[nH]cc(C#N)c12 ; Cc1ccc(NS(=O)(=O)c2ccc(S(=O)(=O)N3CCNCC3)cc2)c2[nH]cc(C#N)c12] 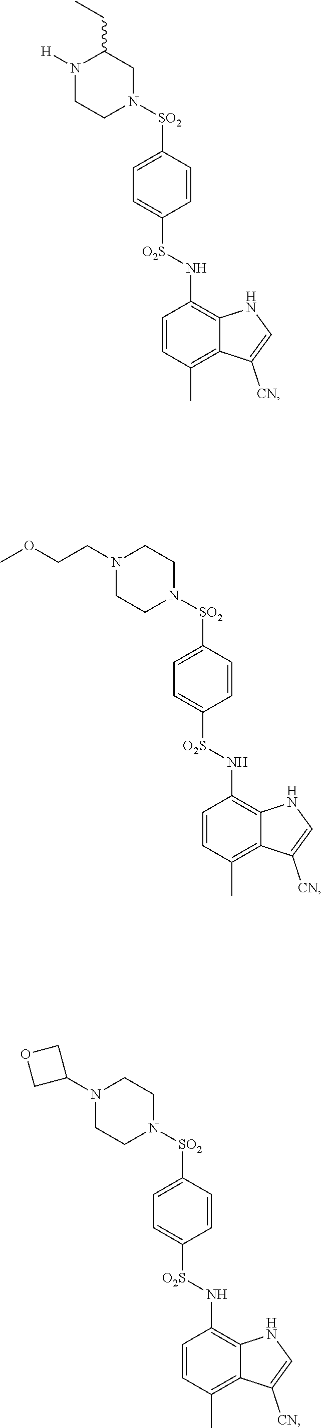 [SMILES: CCC1CN(S(=O)(=O)c2ccc(S(=O)(=O)Nc3ccc(C)c4c(C#N)c[nH]c34)cc2)CCN1 ; COCCN1CCN(S(=O)(=O)c2ccc(S(=O)(=O)Nc3ccc(C)c4c(C#N)c[nH]c34)cc2)CC1 ; Cc1ccc(NS(=O)(=O)c2ccc(S(=O)(=O)N3CCN(C4COC4)CC3)cc2)c2[nH]cc(C#N)c12] 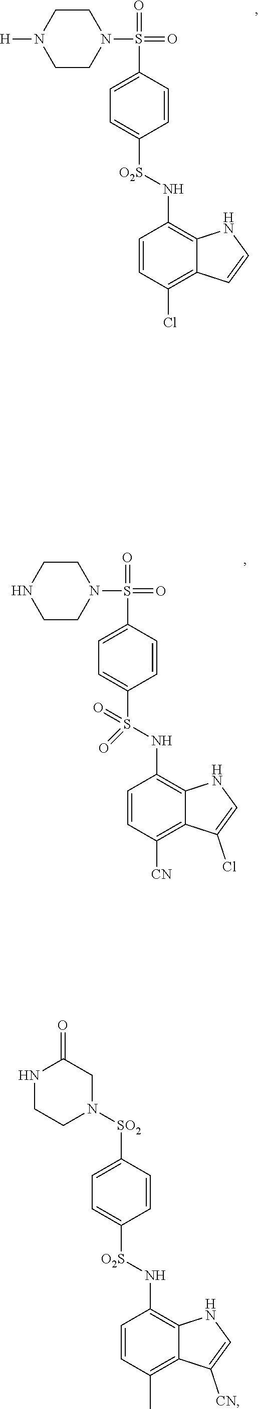 [SMILES: O=S(=O)(Nc1ccc(Cl)c2cc[nH]c12)c1ccc(S(=O)(=O)N2CCNCC2)cc1 ; N#Cc1ccc(NS(=O)(=O)c2ccc(S(=O)(=O)N3CCNCC3)cc2)c2[nH]cc(Cl)c12 ; Cc1ccc(NS(=O)(=O)c2ccc(S(=O)(=O)N3CCNC(=O)C3)cc2)c2[nH]cc(C#N)c12] 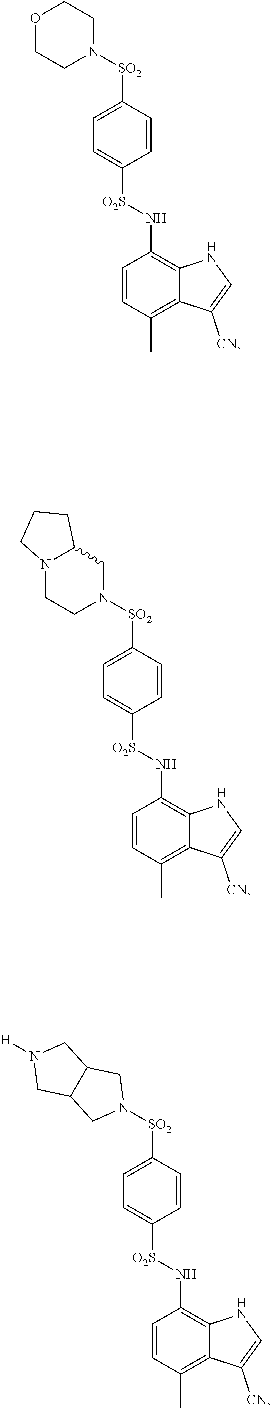 [SMILES: Cc1ccc(NS(=O)(=O)c2ccc(S(=O)(=O)N3CCOCC3)cc2)c2[nH]cc(C#N)c12 ; Cc1ccc(NS(=O)(=O)c2ccc(S(=O)(=O)N3CCN4CCCC4C3)cc2)c2[nH]cc(C#N)c12 ; Cc1ccc(NS(=O)(=O)c2ccc(S(=O)(=O)N3CC4CNCC4C3)cc2)c2[nH]cc(C#N)c12] 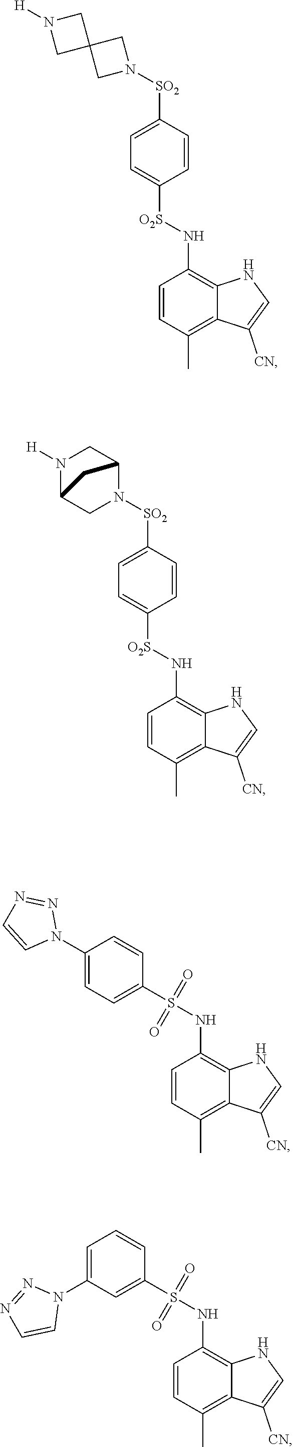 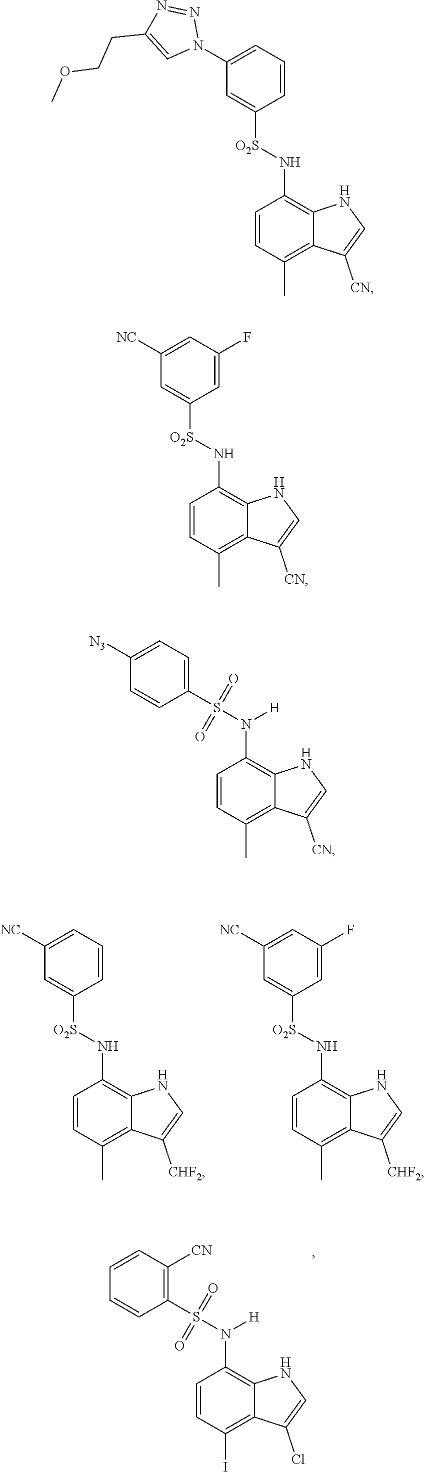 [SMILES: COCCc1cn(-c2cccc(S(=O)(=O)Nc3ccc(C)c4c(C#N)c[nH]c34)c2)nn1 ; Cc1ccc(NS(=O)(=O)c2cc(F)cc(C#N)c2)c2[nH]cc(C#N)c12 ; Cc1ccc(NS(=O)(=O)c2ccc(N=[N+]=[N-])cc2)c2[nH]cc(C#N)c12 ; Cc1ccc(NS(=O)(=O)c2cccc(C#N)c2)c2[nH]cc(C(F)F)c12 ; Cc1ccc(NS(=O)(=O)c2cc(F)cc(C#N)c2)c2[nH]cc(C(F)F)c12 ; N#Cc1ccccc1S(=O)(=O)Nc1ccc(I)c2c(Cl)c[nH]c12] 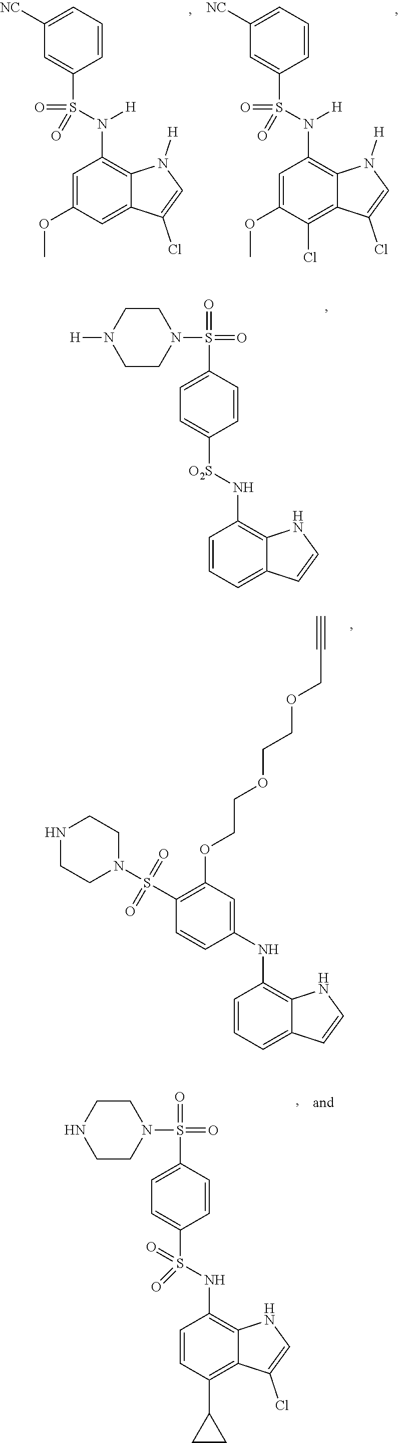 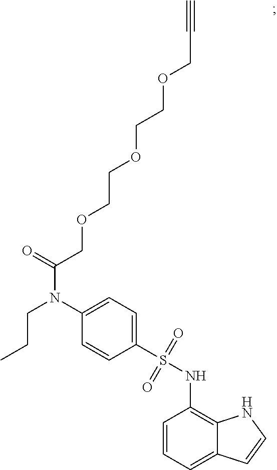 [SMILES: C#CCOCCOCCOCC(=O)N(CCC)c1ccc(S(=O)(=O)Nc2cccc3cc[nH]c23)cc1]
or a pharmaceutically acceptable salt thereof.

23. (canceled)

24. A pharmaceutical composition comprising a therapeutically effective amount of a compound of claim 1.

25-31. (canceled)

32. A method of treating a disease or disorder that results from the function of a target protein in a subject, comprising administering to the subject, a therapeutically effective amount of a compound according to claim 1, or a pharmaceutically acceptable salt thereof, wherein the compound induces degradation of the target protein thereby treating the disease or disorder.

33-45. (canceled)
